(19) United States
(12) Patent Application Publication
(10) Pub. No.: US 2026/0209367 A1
(43) Pub. Date: Jul. 23, 2026

(54) SINGLE IMMUNOGLOBULIN DOMAIN BISPECIFIC ANTIBODIES AGAINST IL 10R alpha AND beta SUBUNITS AND METHODS OF USE

(71) Applicant: Gilead Sciences, Inc., Foster City, CA (US)

(72) Inventors: Chen Chen, Newark, CA (US); Lei Chen, Westborough, MA (US); Georgios Delidakis, San Francisco, CA (US); Huang Huang, San Mateo, CA (US); Bo Jiang, Vista, CA (US); Megan L. Koleske, San Francisco, CA (US); Chingwei V. Lee, Foster City, CA (US); Nicole T. Schirle Oakdale, Castro Valley, CA (US); Nathan D. Thomsen, Castro Valley, CA (US); Hui Xu, Foster City, CA (US); Tangsheng Yi, Belmont, CA (US); Lili Yue, Millbrae, CA (US)

(21) Appl. No.: 19/395,473

(22) Filed: Nov. 20, 2025

Related U.S. Application Data
(60) Provisional application No. 63/724,109, filed on Nov. 22, 2024.

(51) Int. Cl. C07K 16/28; A61K 39/00; A61P 19/02; A61P 29/00

(52) U.S. Cl. CPC C07K 16/2866; A61P 19/02; A61P 29/00; A61K 2039/505; A61K 2039/545; C07K 2317/31; C07K 2317/52; C07K 2317/565; C07K 2317/569; C07K 2317/75; C07K 2317/92

(57) ABSTRACT

Provided are bispecific antibodies comprising first and second VHH binding domains that specifically bind to the extracellular domains of interleukin-10 receptor (IL-10R) alpha subunit (IL-10Rα) and beta subunit (IL-10Rβ), and that selectively suppress activation of myeloid cells and induce minimal or no direct activation of T cells or B cells.

### CROSS-REFERENCE TO RELATED APPLICATIONS

This application claims the benefit under 35 U.S.C. § 119(e) of U.S. Provisional Application No. 63/724,109, filed on Nov. 22, 2024, which is hereby incorporated herein by reference in its entirety for all purposes.

### SEQUENCE LISTING

The instant application contains a Sequence Listing which has been submitted electronically in .XML format and is hereby incorporated by reference in its entirety. Said .XML copy, created on Nov. 7, 2025, is named 1569-US-WO_SL.xml and is 1,039,098 bytes in size.

### BACKGROUND

Interleukin-10 (IL-10) is an immunoregulatory cytokine that plays a role in limiting excessive inflammation and maintaining tissue homeostasis. Despite its potent immunosuppressive effects on myeloid cells, high concentrations of IL-10 can paradoxically activate T-cells, B-cells, and natural killer (NK) cells, counterbalancing its immunosuppressive actions in inflammatory conditions. Additionally, the activation of T-cells and B-cells by IL-10 leads to the production of IFN-γ, increased levels of IgE, a rise in granulocyte-monocyte progenitor (GMP) cells, and a reduction in red blood cells and platelets. These adverse effects of recombinant IL-10 limit its therapeutic potential for treating inflammatory bowel disease (IBD) and psoriasis.

### SUMMARY

In one aspect, provided is a single domain antibody (sdAb) comprising a variable domain (VHH) that specifically binds to the extracellular domain of interleukin-10 receptor (IL-10R) alpha subunit (IL-10Rα). In some embodiments, the anti-IL-10Rα VHH comprises a complementarity determining region (CDR) 1, a VHH-CDR2, and a VHH-CDR3 comprising the following amino acid sequences, according to Kabat, respectively: SEQ ID NOs: 1, 2 and 3 (A01); SEQ ID NOs: 4, 5 and 6 (A06); SEQ ID NOs: 7, 8 and 9 (A09); SEQ ID NOs: 10, 11 and 12 (B07); SEQ ID NOs: 4, 13 and 14 (B08); SEQ ID NOs: 4, 15 and 16 (C09); SEQ ID NOs: 4, 17 and 18 (D08); SEQ ID NOs: 19, 20 and 21 (D14); SEQ ID NOs: 22, 23 and 24 (E07); SEQ ID NOs: 22, 25 and 27 (huE07); SEQ ID NOs: 22, 27 and 24 (huE07); SEQ ID NOs: 4, 28 and 29 (E15); SEQ ID NOs: 4, 30 and 31 (F09); SEQ ID NOs: 22, 23 and 32 (F10); SEQ ID NOs: 33, 34 and 35 (F11); SEQ ID NOs: 4, 36 and 29 (F21); SEQ ID NOs: 19, 37 and 38 (G22); SEQ ID NOs: 39, 40 and 41 (G23); SEQ ID NOs: 42, 43 and 44 (H22); SEQ ID NOs: 45, 46 and 47 (I04); SEQ ID NOs: 4, 48 and 6 (J13); SEQ ID NOs: 4, 49 and 50 (J24); SEQ ID NOs: 19, 51 and 52 (L05); SEQ ID NOs: 19, 37 and 53 (L11) or SEQ ID NOs: 54, 55 and 24 (N16). In some embodiments, the anti-IL-10Rα VHH comprises a complementarity determining region (CDR) 1, a VHH-CDR2, and a VHH-CDR3 comprising the following amino acid sequences, according to IMGT, respectively: SEQ ID NOs: 56, 57 and 58 (A01); SEQ ID NOs: 59, 60 and 61 (A06); SEQ ID NOs: 62, 63 and 64 (A09); SEQ ID NOs: 65, 66 and 67 (B07); SEQ ID NOs: 59, 68 and 69 (B08); SEQ ID NOs: 59, 70 and 71 (C09); SEQ ID NOs: 59, 72 and 73 (D08) SEQ ID NOs: 74, 75 and 76 (D14); SEQ ID NOs: 77, 78 and 79 (E07) SEQ ID NOs: 77, 80 and 81 (huE07); SEQ ID NOs: 77, 82 and 79 (huE07); SEQ ID NOs: 59, 83 and 84 (E15); SEQ ID NOs: 59, 85 and 86 (F09); SEQ ID NOs: 77, 78 and 87 (F10); SEQ ID NOs: 88, 89 and 90 (F11); SEQ ID NOs: 59, 91 and 84 (F21); SEQ ID NOs: 92, 75 and 93 (G22); SEQ ID NOs: 94, 89 and 95 (G23); SEQ ID NOs: 96, 89 and 97 (H22); SEQ ID NOs: 98, 99 and 100 (I04); SEQ ID NOs: 59, 101 and 61 (J13); SEQ ID NOs: 59, 102 and 103 (J24); SEQ ID NOs: 74, 75 and 104 (L05); SEQ ID NOs: 74, 75 and 105 (L11) or SEQ ID NOs: 106, 107 and 79 (N16). In some embodiments, the anti-IL-10Rα VHH comprises a complementarity determining region (CDR) 1, a VHH-CDR2, and a VHH-CDR3 comprising the following amino acid sequences, according to Chothia, respectively: SEQ ID NOs: 108, 109 and 110 (A01); SEQ ID NOs: 111, 112 and 113 (A06); SEQ ID NOs: 114, 115 and 116 (A09); SEQ ID NOs: 117, 118 and 119 (B07); SEQ ID NOs: 111, 112 and 120 (B08); SEQ ID NOs: 111, 121 and 122 (C09); SEQ ID NOs: 111, 121 and 123 (D08); SEQ ID NOs: 124, 125 and 126 (D14); SEQ ID NOs: 127, 128 and 129 (E07); SEQ ID NOs: 127, 130 and 131 (huE07); SEQ ID NOs: 111, 132 and 133 (E15); SEQ ID NOs: 111, 134 and 135 (F09); SEQ ID NOs: 127, 128 and 136 (F10); SEQ ID NOs: 137, 138 and 139 (F11); SEQ ID NOs: 111, 132 and 133 (F21); SEQ ID NOs: 140, 125 and 141 (G22); SEQ ID NOs: 142, 138 and 143 (G23); SEQ ID NOs: 124, 138 and 144 (H22); SEQ ID NOs: 145, 146 and 147 (I04); SEQ ID NOs: 111, 112 and 113 (J13)); SEQ ID NOs: 111, 148 and 149 (J24); SEQ ID NOs: 124, 125 and 150; (L05); SEQ ID NOs: 124, 125 and 151; (L11) or; SEQ ID NOs: 152, 121 and 129 (N16). In some embodiments, the anti-IL-10Rα VHH comprising a complementarity determining region (CDR) 1, a VHH-CDR2, and a VHH-CDR3 comprising the following amino acid sequences, according to Honegger, respectively: SEQ ID NOs: 153, 154 and 155 (A01); SEQ ID NOs: 156, 157 and 158 (A06); SEQ ID NOs: 159, 160 and 161 (A09); SEQ ID NOs: 162, 163 and 164 (B07); SEQ ID NOs: 156, 165 and 166 (B08); SEQ ID NOs: 156, 167 and 168 (C09); SEQ ID NOs: 156, 169 and 170 (D08); SEQ ID NOs: 171, 172 and 173 (D14); SEQ ID NOs: 174, 175 and 176 (E07); SEQ ID NOs: 174, 177 and 178 (huE07); SEQ ID NOs: 174, 179 and 176 (huE07); SEQ ID NOs: 156, 180 and 181; (E15); SEQ ID NOs: 156, 182 and 183; (F09); SEQ ID NOs: 174, 175 and 184; (F10); SEQ ID NOs: 185, 186 and 187; (F11); SEQ ID NOs: 709, 186 and 187; (huF11); SEQ ID NOs: 156, 188 and 181; (F21); SEQ ID NOs: 189, 190 and 191; (G22); SEQ ID NOs: 192, 193 and 194; (G23); SEQ ID NOs: 195, 196 and 197; (H22); SEQ ID NOs: 198, 199 and 200; (I04); SEQ ID NOs: 156, 201 and 158; (J13); SEQ ID NOs: 156, 202 and 203; (J24); SEQ ID NOs: 204, 205 and 206; (L05); SEQ ID NOs: 204, 190 and 207; (L11) or; SEQ ID NOs: 208, 209 and 176 (N16). In some embodiments, the anti-IL-10Rα VHH comprises a VHH-CDR1, a VHH-CDR2, and a VHH-CDR3 comprising the following amino acid sequences, respectively: SEQ ID NOs: 22, 23 and 24; (E07; according to Kabat); SEQ ID NOs: 22, 25 and 27; (huE07; according to Kabat); SEQ ID NOs: 22, 27 and 24; (huE07; according to Kabat); SEQ ID NOs: 77, 78 and 79; (E07; according to IMGT); SEQ ID NOs: 77, 80 and 81; (huE07; according to IMGT); SEQ ID NOs: 77, 82 and 79; (huE07; according to IMGT); SEQ ID NOs: 127, 128 and 129; (E07; according to Chothia); SEQ ID NOs: 127, 130 and 131; (huE07; according to Chothia); SEQ ID NOs: 174, 175 and 176; (E07; according to Honegger); SEQ ID NOs: 174, 177 and 178; (huE07; according to Honegger) or SEQ ID NOs: 174, 179 and 176; (huE07; according to Honegger). In some embodiments, the anti-IL-10Rα VHH comprises a VHH-CDR1, a VHH-CDR2, and a VHH-CDR3 comprising the following amino acid sequences, respectively: SEQ ID NOs: 33, 34 and 35 (F11; according to Kabat); SEQ ID NOs: 88, 89 and 90 (F11; according to IMGT); SEQ ID NOs: 137, 138 and 139 (F11; according to Chothia); SEQ ID NOs: 185, 186 and 187 (F11; according to Honegger) or SEQ ID NOs: 709, 186 and 187 (huF11; according to Honegger). In some embodiments, the anti-IL-10Rα VHH comprises the amino acid sequence of any one of SEQ ID NOs: 212, 230, 233, 234, 249 and 710, or an amino acid sequence that is at least 80%, at least 85%, at least 90%, at least 91%, at least 92%, at least 93%, at least 94%, at least 95%, at least 96%, at least 97%, at least 98%, or at least 99% identical, to one of the amino acid sequences of SEQ ID NOs: 212, 230, 233, 234, 249 and 710. In some embodiments, the anti-IL-10Rα VHH comprises the amino acid sequence of any one of 210-211, 213-229, 231-232, 235-248, 250-287 and 752-755, or an amino acid sequence that is at least 80%, at least 85%, at least 90%, at least 91%, at least 92%, at least 93%, at least 94%, at least 95%, at least 96%, at least 97%, at least 98%, or at least 99% identical, to one of the amino acid sequences of SEQ ID NOs: 210-211, 213-229, 231-232, 235-248, 250-287 and 752-755. In some embodiments, the anti-IL-10Rα VHH comprises the amino acid sequence of any one of SEQ ID NOs: 233-240 and 724-725, or an amino acid sequence that is at least 80%, at least 85%, at least 90%, at least 91%, at least 92%, at least 93%, at least 94%, at least 95%, at least 96%, at least 97%, at least 98%, or at least 99% identical, to one of the amino acid sequences of SEQ ID NOs: 233-240 and 724-725 (E07). In some embodiments, the anti-IL-10Rα VHH comprises the amino acid sequence of any one of SEQ ID NOs: 249-252 and 752-755, or an amino acid sequence that is at least 80%, at least 85%, at least 90%, at least 91%, at least 92%, at least 93%, at least 94%, at least 95%, at least 96%, at least 97%, at least 98%, or at least 99% identical, to one of the amino acid sequences of SEQ ID NOs: 249-252 and 752-755 (F11). In some embodiments, the anti-IL-10Rα VHH is humanized. In some embodiments, the anti-IL-10Rα VHH binds to an epitope on IL 10Rα comprising contact amino acid residues W33, E35, E37, H40, 142, H44, T46, L79, S80, T124, A170, R172, F178, F180, H182, Q204, K215, M217, W218, K220 and E221, wherein the amino acid residue positions are with respect to SEQ ID NO: 711. In some embodiments, the anti-IL-10Rα VHH binds to an epitope on IL 10Rα comprising contact amino acid residues I169, I171, K173, F178, F180, T181, H182, K183, K186, F190, L192, L193, S195, E197 and F201, wherein the amino acid residue positions are with respect to SEQ ID NO: 711. In some embodiments, the anti-IL-10Rα VHH binds to IL 10Rα with an equilibrium dissociation constant (KD) of lower than or less than 5 nM, e.g., lower than 4 nM, 3 nM or 2 nM. In some embodiments, the VHH that binds to IL 10Rα binds to both human IL-10Rα and cynomolgus IL-10Rα. In some embodiments, the anti-IL-10Rα sdAb comprises a human fragment crystallizable (Fc) domain. In some embodiments, the Fc domain comprises a human IgG1 or a human IgG4.

In another aspect, provided is a single domain antibody (sdAb) comprising a variable domain (VHH) that specifically binds to the extracellular domain of interleukin-10 receptor (IL-10R) beta subunit (IL 10Rβ). In some embodiments, the anti-IL-10Rβ VHH comprises a complementarity determining region (CDR) 1, a VHH-CDR2, and a VHH-CDR3 comprising the following amino acid sequences, according to Kabat, respectively: SEQ ID NOs: 288, 289 and 290 (A02); SEQ ID NOs: 291, 292 and 293 (A05); SEQ ID NOs: 291, 294 and 295 (huA05); SEQ ID NOs: 296, 297 and 298 (huA05); SEQ ID NOs: 299, 300 and 301 (B10); SEQ ID NOs: 302, 303 and 304 (B12); SEQ ID NOs: 305, 306 and 307 (C01); SEQ ID NOs: 296, 308 and 309 (C04); SEQ ID NOs: 310, 311 and 312 (C10); SEQ ID NOs: 302, 303 and 313 (D01); SEQ ID NOs: 314, 315 and 316 (E08); SEQ ID NOs: 317, 318 and 319 (E10); SEQ ID NOs: 320, 321 and 322 (G01) or; SEQ ID NOs: 323, 324 and 325 (G02). In some embodiments, the anti-IL-10Rβ VHH comprises a complementarity determining region (CDR) 1, a VHH-CDR2, and a VHH-CDR3 comprising the following amino acid sequences, according to IMGT, respectively: SEQ ID NOs: 326, 327 and 328 (A02); SEQ ID NOs: 329, 330 and 331 (A05); SEQ ID NOs: 329, 332 and 333 (huA05); SEQ ID NOs: 334, 335 and 336 (huA05); SEQ ID NOs: 337, 338 and 339 (B10); SEQ ID NOs: 340, 341 and 342 (B12); SEQ ID NOs: 343, 344 and 345 (C01); SEQ ID NOs: 346, 347 and 348 (C04); SEQ ID NOs: 349, 350 and 351 (C10); SEQ ID NOs: 352, 341 and 353 (D01); SEQ ID NOs: 354, 355 and 356 (E08); SEQ ID NOs: 357, 358 and 359 (E10); SEQ ID NOs: 360, 361 and 362 (G01) or SEQ ID NOs: 363, 364 and 365 (G02). In some embodiments, the anti-IL-10Rβ VHH comprises a complementarity determining region (CDR) 1, a VHH-CDR2, and a VHH-CDR3 comprising the following amino acid sequences, according to Chothia, respectively: SEQ ID NOs: 366, 367 and 368 (A02); SEQ ID NOs: 369, 370 and 371 (A05); SEQ ID NOs: 369, 372 and 373 (huA05); SEQ ID NOs: 374, 375 and 376 (huA05); SEQ ID NOs: 377, 378 and 379 (B10); SEQ ID NOs: 380, 381 and 382 (B12); SEQ ID NOs: 383, 384 and 385 (C01); SEQ ID NOs: 386, 387 and 388 (C04); SEQ ID NOs: 389, 390 and 391 (C10); SEQ ID NOs: 392, 381 and 393 (D01); SEQ ID NOs: 394, 395 and 396 (E08); SEQ ID NOs: 397, 398 and 399 (E10); SEQ ID NOs: 400, 401 and 402 (G01) or SEQ ID NOs: 403, 404 and 405 (G02). In some embodiments, the anti-IL-10Rβ VHH comprises a complementarity determining region (CDR) 1, a VHH-CDR2, and a VHH-CDR3 comprising the following amino acid sequences, according to Honegger, respectively: SEQ ID NOs: 406, 407 and 408 (A02); SEQ ID NOs: 409, 410 and 411 (A05); SEQ ID NOs: 409, 412 and 413 (huA05); SEQ ID NOs: 414, 415 and 416 (huA05); SEQ ID NOs: 417, 418 and 419 (B10); SEQ ID NOs: 420, 421 and 422 (B12); SEQ ID NOs: 423, 424 and 425 (C01); SEQ ID NOs: 426, 427 and 428 (C04); SEQ ID NOs: 429, 430 and 431 (C10); SEQ ID NOs: 432, 421 and 433 (D01); SEQ ID NOs: 434, 435 and 436 (E08); SEQ ID NOs: 437, 438 and 439 (E10); SEQ ID NOs: 440, 441 and 442 (G01) or; SEQ ID NOs: 443, 444 and 445 (G02). In some embodiments, the anti-IL-10Rβ VHH comprises a VHH-CDR1, a VHH-CDR2, and a VHH-CDR3 comprising the following amino acid sequences, respectively: SEQ ID NOs: 291, 292 and 293 (A05; according to Kabat); SEQ ID NOs: 291, 294 and 295 (huA05; according to Kabat); SEQ ID NOs: 296, 297 and 298 (huA05; according to Kabat); SEQ ID NOs: 329, 330 and 331 (A05; according to IMGT); SEQ ID NOs: 329, 332 and 333 (huA05; according to IMGT); SEQ ID NOs: 334, 335 and 336 (huA05; according to IMGT); SEQ ID NOs: 369, 370 and 371 (A05; according to Chothia); SEQ ID NOs: 369, 372 and 373 (huA05; according to Chothia); SEQ ID NOs: 374, 375 and 376 (huA05; according to Chothia); SEQ ID NOs: 409, 410 and 411 (A05; according to Honegger); SEQ ID NOs: 409, 412 and 413 (huA05; according to Honegger) or SEQ ID NOs: 414, 415 and 416 (huA05; according to Honegger). In some embodiments, the anti-IL-10Rβ VHH comprises the amino acid sequence of any one of SEQ ID NOs: 448, 449, 455 and 460, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 448, 449, 455 and 460. In some embodiments, the anti-IL-10Rβ VHH comprises the amino acid sequence of any one of SEQ ID NOs: 446-447, 450-454, 456-459 and 461-474, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 446-447, 450-454, 456-459 and 461-474. In some embodiments, the anti-IL-10Rβ VHH comprises the amino acid sequence of any one of SEQ ID NOs: 448-452, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 448-452 (A05). In some embodiments, the anti-IL-10Rβ VHH comprises the amino acid sequence of any one of SEQ ID NOs: 460-462, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 460-462 (C04). In some embodiments, the anti-IL-10Rβ VHH is humanized. In some embodiments, the anti-IL-10Rβ VHH binds to an epitope on IL-10Rβ comprising contact amino acid residues S78, S80, K81, Y82, V108, N138, E139, Y140, E141, T142, W143, N147, V148 and N150, wherein the amino acid residue positions are with respect to SEQ ID NO: 712. In some embodiments, the anti-IL-10Rβ VHH binds to an epitope on IL-10Rβ comprising contact amino acid residues N26, R28, N30, V32, Q39, E41, T72, D84, 1103, T104, D110, 1112, P115, Q157, Q190, R192, F194, N199, A201, E203, W204 and E206, wherein the amino acid residue positions are with respect to SEQ ID NO: 712. In some embodiments, the anti-IL-10Rβ VHH binds to IL 10Rβ with an equilibrium dissociation constant (KD) of greater than 10 nM, e.g., greater than 20 nM, 30 nM, 40 nM or 50 nM. In some embodiments, the VHH that binds to IL 10Rβ binds to both human IL-10Rβ and cynomolgus IL-10Rβ. In some embodiments, the anti-IL-10Rβ sdAb comprises a human fragment crystallizable (Fc) domain. In some embodiments, the Fc domain comprises a human IgG1 or a human IgG4.

Further provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the bispecific antibody is an IL-10R agonist that selectively suppresses activation of myeloid cells and induces minimal or no direct activation of one or both of T cells and B cells. In some embodiments, the bispecific antibody is an IL-10R agonist that predominantly suppresses activation of myeloid cells and induces minimal or no direct activation of one or both of T cells and B cells. In some embodiments, the bispecific antibody induces a maximum effective (Emax) activation of one or both of T cells and B cells that is 15% or less, e.g., 10% or less, of wild-type IL-10. In some embodiments, the bispecific antibody is an IL-10R agonist that predominantly or selectively suppresses activation of myeloid cells and induces a maximum effect (Emax) activation of one or both of T cells and B cells that is 15% or less, e.g., 10% or less, of wild-type IL-10. In some embodiments, the bispecific antibody is an IL-10R agonist suppressing activation of myeloid cells with a half maximal effective concentration (EC50) less than 10 pM and activating one or both of T cells and B cells below a predetermined threshold level at a concentration above 1000 nM. In some embodiments, the bispecific antibody activates T cells or B cells with a maximum effect (Emax) that is 15% or less, e.g., 10% or less, of wild-type IL-10. In some embodiments, the activation of T cells is measured by at least one of Granzyme B secretion and interferon-γ (IFN-γ) production. In some embodiments, the myeloid cells are selected from macrophages, monocytes and dendritic cells. In some embodiments, the bispecific antibody does not induce detectable granzyme B secretion from CD8+ T cells. In some embodiments, the bispecific antibody induces interferon-γ (IFN-γ) from CD8+ T cells less than 15%, e.g., less than 10%, the level of IFN-γ induced by wild-type IL-10. In some embodiments, the bispecific antibody induces minimal (e.g., less than 25%, less than 20%, less than 15%, less than 10%, in comparison to wild-type IL-10) or no inhibition of IL 10Rβ dimerization with other IL-10 family cytokine receptor subunits, e.g., IL-10Rβ dimerization with IL-20Rα, IL-22R, IL-28R or IL-29R. In some embodiments, the bispecific antibody agonizes the IL-10 receptor in a tyrosine kinase 2 (TYK2; NCBI Gene ID: 7297) dependent manner. In some embodiments, the bispecific antibody induces minimal (e.g., less than 25%, less than 20%, less than 15%, less than 10%, in comparison to wild-type IL-10) or no inhibition of intracellular signaling of other IL 10Rβ-binding cytokines, e.g., minimal or no inhibition of IL-22, IL-26, IL-28A, IL-28B, or IL-29 intracellular signaling. In some embodiments, the second VHH binds to IL 10Rβ with an equilibrium dissociation constant (KD) that is at least 5-fold greater, e.g., at least 7-fold greater, e.g., at least 10-fold, 15-fold, 20-fold, 25-fold greater, or more, than the KD of the first VHH binding to IL 10Rα, e.g., wherein the binding affinity is determined by surface plasmon resonance (SPR). In some embodiments, the first VHH binds to IL 10Rα with affinity that is at least 5-fold greater, e.g., at least 7-fold greater, e.g., at least 10-fold, 15-fold, 20-fold, 25-fold greater, or more, than the binding affinity of the second VHH binding to IL 10Rβ, e.g., wherein the binding affinity is determined by surface plasmon resonance (SPR). In some embodiments, the first variable domain binds to IL 10Rα with an equilibrium dissociation constant (KD) of lower than 2 nM and the second variable domain binds to IL 10Rβ with a KD of greater than 10 nM, e.g., wherein the binding affinity is determined by surface plasmon resonance (SPR). In some embodiments, the first variable domain binds to IL 10Rα with an equilibrium dissociation constant (KD) of lower than 2 nM and the second variable domain binds to IL 10Rβ with a KD of greater than 50 nM, e.g., wherein the binding affinity is determined by surface plasmon resonance (SPR). In some embodiments, the first VHH binds to an epitope on IL 10Rα comprising contact amino acid residues W33, E35, E37, H40, 142, H44, T46, L79, 580, T124, A170, R172, F178, F180, H182, Q204, K215, M217, W218, K220 and E221, wherein the amino acid residue positions are with respect to SEQ ID NO: 711 and the second VHH binds to an epitope on IL-10Rβ comprising contact amino acid residues S78, S80, K81, Y82, V108, N138, E139, Y140, E141, T142, W143, N147, V148 and N150, wherein the amino acid residue positions are with respect to SEQ ID NO: 712. In some embodiments, the first VHH binds to an epitope on IL-10Rα comprising contact amino acid residues I169, I171, K173, F178, F180, T181, H182, K183, K186, F190, L192, L193, S195, E197, and F201, wherein the amino acid residue positions are with respect to SEQ ID NO: 711 and the second VHH binds to an epitope on IL-10Rβ comprising contact amino acid residues N26, R28, N30, V32, Q39, E41, T72, D84, 1103, T104, D110, I112, P115, Q157, Q190, R192, F194, N199, A201, E203, W204, and E206, wherein the amino acid residue positions are with respect to SEQ ID NO: 712. In some embodiments, the bispecific antibody comprises at least one polypeptide (e.g., two polypeptides) comprising the first VHH in tandem with the second VHH. In some embodiments, the bispecific antibody comprises at least one polypeptide comprising from N-terminus to C-terminus the first VHH that specifically binds to IL-10Rα and the second VHH that specifically binds to IL-10Rβ. In some embodiments, the bispecific antibody comprises at least one polypeptide comprising from N-terminus to C-terminus the second VHH that specifically binds to IL-10Rβ and the first VHH that specifically binds to IL-10Rα. In some embodiments, the first VHH and the second VHH are connected via a linker. In some embodiments, the linker is selected from the group consisting of: GS (SEQ ID NO: 713), GGS (SEQ ID NO: 714), GGGS (SEQ ID NO: 715), GGSGGS (SEQ ID NO: 716), GGGSGGGS (SEQ ID NO: 717) and GGSGGSGGS (SEQ ID NO: 718). In some embodiments, the linker comprises GGGS (SEQ ID NO: 715). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to Kabat, respectively: SEQ ID NOs: 22, 23, 24, 291, 292 and 293 (877); SEQ ID NOs: 22, 25, 26, 291, 294 and 295 (647); SEQ ID NOs: 22, 27, 24, 291, 297 and 298 (195); SEQ ID NOs: 22, 23, 24, 288, 289 and 290 (048); SEQ ID NOs: 22, 25, 26, 288, 289 and 290 (049); SEQ ID NOs: 22, 27, 24, 288, 289 and 290 (050); SEQ ID NOs: 22, 23, 24, 296, 308 and 309 (274); SEQ ID NOs: 22, 25, 26, 296, 308 and 309 (771); SEQ ID NOs: 22, 27, 24, 296, 308 and 309 (772); SEQ ID NOs: 4, 28, 29, 288, 289 and 290 (054); SEQ ID NOs: 4, 28, 29, 291, 294 and 295 (883); SEQ ID NOs: 4, 28, 29, 291, 294 and 295 (784); SEQ ID NOs: 4, 28, 29, 291, 297 and 298 (785); SEQ ID NOs: 4, 5, 6, 288, 289 and 290 (046); SEQ ID NOs: 4, 5, 6, 291, 294 and 295 (875); SEQ ID NOs: 4, 5, 6, 291, 294 and 295 (776); SEQ ID NOs: 4, 5, 6, 291, 297 and 298 (777); SEQ ID NOs: 4, 5, 6, 296, 308 and 309 (272); SEQ ID NOs: 4, 15, 16, 288, 289 and 290 (052); SEQ ID NOs: 4, 15, 16, 296, 308 and 309 (278); SEQ ID NOs: 4, 17, 18, 288, 289 and 290 (047); SEQ ID NOs: 4, 17, 18, 291, 292 and 293 (876); SEQ ID NOs: 4, 17, 18, 291, 294 and 295 (778); SEQ ID NOs: 4, 17, 18, 291, 297 and 298 (779); SEQ ID NOs: 45, 46, 47, 288, 289 and 290; (062); SEQ ID NOs: 45, 46, 47, 291, 292 and 293 (891); SEQ ID NOs: 45, 46, 47, 291, 294 and 295 (780); SEQ ID NOs: 45, 46, 47, 291, 297 and 298 (781); SEQ ID NOs: 45, 46, 47, 296, 308 and 309 (290); SEQ ID NOs: 4, 48, 6, 291, 292 and 293 (892); SEQ ID NOs: 4, 48, 6, 291, 294 and 295 (793); SEQ ID NOs: 4, 48, 6, 291, 297 and 298 (794); SEQ ID NOs: 54, 55, 24, 291, 292 and 293 (894); SEQ ID NOs: 54, 55, 24, 291, 294 and 295 (795); SEQ ID NOs: 54, 55, 24, 291, 297 and 298 (796); SEQ ID NOs: 4, 30, 31, 291, 292 and 293 (884); SEQ ID NOs: 4, 30, 31, 291, 294 and 295 (786); SEQ ID NOs: 4, 30, 31, 291, 297 and 298 (787); SEQ ID NOs: 4, 36, 29, 288, 289 and 290 (058); SEQ ID NOs: 33, 34, 35, 296, 308 and 309 (284); SEQ ID NOs: 33, 34, 35, 296, 308 and 309 (183); SEQ ID NOs: 33, 34, 35, 296, 308 and 309 (184); SEQ ID NOs: 19, 37, 38, 302, 303 and 304 (239); SEQ ID NOs: 19, 37, 38, 296, 308 and 309 (286) or SEQ ID NOs: 19, 51, 52, 302, 303 and 304 (247). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to IMGT, respectively: SEQ ID NOs: 77, 78, 79, 329, 330 and 331 (877); SEQ ID NOs: 77, 80, 81, 329, 332 and 333 (647); SEQ ID NOs: 77, 82, 79, 334, 335 and 336 (195); SEQ ID NOs: 77, 78, 79, 326, 327 and 328 (048); SEQ ID NOs: 77, 80, 81, 326, 327 and 328 (049); SEQ ID NOs: 77, 82, 79, 326, 327 and 328 (050); SEQ ID NOs: 77, 78, 79, 346, 347 and 348 (274); SEQ ID NOs: 77, 80, 81, 346, 347 and 348 (771); SEQ ID NOs: 77, 82, 79, 346, 347 and 348 (772); SEQ ID NOs: 59, 83, 84, 326, 327 and 328 (054); SEQ ID NOs: 59, 83, 84, 329, 330 and 331 (883); SEQ ID NOs: 59, 83, 84, 329, 332 and 333 (784); SEQ ID NOs: 59, 83, 84, 334, 335 and 336 (785); SEQ ID NOs: 59, 60, 61, 326, 327 and 328 (046); SEQ ID NOs: 59, 60, 61, 329, 330 and 331 (875); SEQ ID NOs: 59, 60, 61, 329, 332 and 333 (776); SEQ ID NOs: 59, 60, 61, 334, 335 and 336 (777); SEQ ID NOs: 59, 60, 61, 346, 347 and 348 (272); SEQ ID NOs: 59, 70, 71, 326, 327 and 328 (052); SEQ ID NOs: 59, 70, 71, 346, 347 and 348 (278); SEQ ID NOs: 59, 72, 73, 326, 327 and 328 (047); SEQ ID NOs: 59, 72, 73, 329, 330 and 331 (876); SEQ ID NOs: 59, 72, 73, 329, 332 and 333 (778); SEQ ID NOs: 59, 72, 73, 334, 335 and 336 (779); SEQ ID NOs: 98, 99, 100, 326, 327 and 328 (062); SEQ ID NOs: 98, 99, 100, 329, 330 and 331 (891); SEQ ID NOs: 98, 99, 100, 329, 332 and 333 (780); SEQ ID NOs: 98, 99, 100, 334, 335 and 336 (781); SEQ ID NOs: 98, 99, 100, 326, 327 and 328 (290); SEQ ID NOs: 59, 101, 61, 329, 330 and 331 (892); SEQ ID NOs: 59, 101, 61, 329, 332 and 333 (793); SEQ ID NOs: 59, 101, 61, 334, 335 and 336 (794); SEQ ID NOs: 106, 107, 79, 329, 330 and 331 (894); SEQ ID NOs: 106, 107, 79, 329, 332 and 333 (795); SEQ ID NOs: 106, 107, 79, 334, 335 and 336 (796); SEQ ID NOs: 59, 85, 86, 329, 330 and 331 (884); SEQ ID NOs: 59, 85, 86, 329, 332 and 333 (786); SEQ ID NOs: 59, 85, 86, 334, 335 and 336 (787); SEQ ID NOs: 59, 91, 84, 326, 327 and 328 (058); SEQ ID NOs: 88, 89, 90, 326, 327 and 328 (284); SEQ ID NOs: 88, 89, 90, 326, 327 and 328 (183); SEQ ID NOs: 88, 89, 90, 326, 327 and 328 (184); SEQ ID NOs: 92, 75, 93, 340, 341 and 342 (239); SEQ ID NOs: 92, 75, 93, 326, 327 and 328 (286) or SEQ ID NOs: 74, 75, 104, 340, 341 and 342 (247). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to Chothia, respectively: SEQ ID NOs: 127, 128, 129, 369, 370 and 371 (877); SEQ ID NOs: 127, 130, 131, 369, 372 and 373 (647); SEQ ID NOs: 127, 130, 131, 374, 375 and 376 (195); SEQ ID NOs: 127, 128, 129, 366, 367 and 368 (048); SEQ ID NOs: 127, 130, 131, 366, 367 and 368 (049); SEQ ID NOs: 127, 130, 131, 366, 367 and 368 (050); SEQ ID NOs: 127, 128, 129, 386, 387 and 388 (274); SEQ ID NOs: 127, 130, 131, 386, 387 and 388 (771); SEQ ID NOs: 127, 130, 131, 386, 387 and 388 (772); SEQ ID NOs: 111, 132, 133, 366, 367 and 368 (054); SEQ ID NOs: 111, 132, 133, 369, 370 and 371 (883); SEQ ID NOs: 111, 132, 133, 369, 372 and 373 (784); SEQ ID NOs: 111, 132, 133, 374, 375 and 376 (785); SEQ ID NOs: 111, 112, 113, 366, 367 and 368 (046); SEQ ID NOs: 111, 112, 113, 369, 370 and 371 (875); SEQ ID NOs: 111, 112, 113, 369, 372 and 373 (776); SEQ ID NOs: 111, 112, 113, 374, 375 and 376 (777); SEQ ID NOs: 111, 112, 113, 386, 387 and 388 (272); SEQ ID NOs: 111, 121, 122, 366, 367 and 368 (052); SEQ ID NOs: 111, 121, 122, 386, 387 and 388 (278); SEQ ID NOs: 111, 121, 123, 366, 367 and 368 (047); SEQ ID NOs: 111, 121, 123, 369, 370 and 371 (876); SEQ ID NOs: 111, 121, 123, 369, 372 and 373 (778); SEQ ID NOs: 111, 121, 123, 374, 375 and 376 (779); SEQ ID NOs: 145, 146, 147, 366, 367 and 368 (062); SEQ ID NOs: 145, 146, 147, 369, 370 and 371 (891); SEQ ID NOs: 145, 146, 147, 369, 372 and 373 (780); SEQ ID NOs: 145, 146, 147, 374, 375 and 376 (781); SEQ ID NOs: 145, 146, 147, 386, 387 and 388 (290); SEQ ID NOs: 111, 112, 113, 369, 370 and 371 (892); SEQ ID NOs: 111, 112, 113, 369, 372 and 373 (793); SEQ ID NOs: 111, 112, 113, 374, 375 and 376 (794); SEQ ID NOs: 152, 121, 129, 369, 370 and 371 (894); SEQ ID NOs: 152, 121, 129, 369, 372 and 373 (795); SEQ ID NOs: 152, 121, 129, 374, 375 and 376 (796); SEQ ID NOs: 111, 134, 135, 369, 370 and 371 (884); SEQ ID NOs: 111, 134, 135, 369, 372 and 373 (786); SEQ ID NOs: 111, 134, 135, 374, 375 and 376 (787); SEQ ID NOs: 111, 132, 133, 366, 367 and 368 (058); SEQ ID NOs: 137, 138, 139, 386, 387 and 388 (284); SEQ ID NOs: 137, 138, 139, 386, 387 and 388 (183); SEQ ID NOs: 137, 138, 139, 386, 387 and 388 (184); SEQ ID NOs: 140, 125, 141, 380, 381 and 381 (239); SEQ ID NOs: 140, 125, 141, 386, 387 and 388 (286) or SEQ ID NOs: 124, 125, 126, 380, 381 and 381 (247). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to Honegger, respectively: SEQ ID NOs: 174, 175, 176, 409, 410 and 411 (877); SEQ ID NOs: 174, 177, 178, 409, 412 and 413 (647); SEQ ID NOs: 174, 179, 176, 414, 415 and 416 (195); SEQ ID NOs: 174, 175, 176, 406, 407 and 408 (048); SEQ ID NOs: 174, 177, 178, 406, 407 and 408 (049); SEQ ID NOs: 174, 179, 176, 406, 407 and 408 (050); SEQ ID NOs: 174, 175, 176, 426, 427 and 428 (274); SEQ ID NOs: 174, 177, 178, 426, 427 and 428 (771); SEQ ID NOs: 174, 179, 176, 426, 427 and 428 (772); SEQ ID NOs: 156, 180, 181, 406, 407 and 408 (054); SEQ ID NOs: 156, 180, 181, 409, 410 and 411 (883); SEQ ID NOs: 156, 180, 181, 409, 412 and 413 (784); SEQ ID NOs: 156, 180, 181, 414, 415 and 416 (785); SEQ ID NOs: 156, 157, 158, 406, 407 and 408 (046); SEQ ID NOs: 156, 157, 158, 409, 410 and 411 (875); SEQ ID NOs: 156, 157, 158, 409, 412 and 413 (776); SEQ ID NOs: 156, 157, 158, 414, 415 and 416 (777); SEQ ID NOs: 156, 157, 158, 426, 427 and 428 (272); SEQ ID NOs: 156, 167, 168, 406, 407 and 408 (052); SEQ ID NOs: 156, 167, 168, 426, 427 and 428 (278); SEQ ID NOs: 156, 169, 170, 406, 407 and 408 (047); SEQ ID NOs: 156, 169, 170, 409, 410 and 411 (876); SEQ ID NOs: 156, 169, 170, 409, 412 and 413 (778); SEQ ID NOs: 156, 169, 170, 414, 415 and 416 (779); SEQ ID NOs: 198, 199, 200, 406, 407 and 408 (062); SEQ ID NOs: 198, 199, 200, 409, 410 and 411 (891); SEQ ID NOs: 198, 199, 200, 409, 412 and 413 (780); SEQ ID NOs: 198, 199, 200, 414, 415 and 416 (781); SEQ ID NOs: 198, 199, 200, 426, 427 and 428 (290); SEQ ID NOs: 156, 201, 158, 409, 410 and 411 (892); SEQ ID NOs: 156, 201, 158, 409, 412 and 413 (793); SEQ ID NOs: 156, 201, 158, 414, 415 and 416 (794); SEQ ID NOs: 208, 209, 176, 409, 410 and 411 (894); SEQ ID NOs: 208, 209, 176, 409, 412 and 413 (795); SEQ ID NOs: 208, 209, 176, 414, 415 and 416 (796); SEQ ID NOs: 156, 182, 183, 409, 410 and 411 (884); SEQ ID NOs: 156, 182, 183, 409, 412 and 413 (786); SEQ ID NOs: 156, 182, 183, 414, 415 and 416 (787); SEQ ID NOs: 156, 188, 181, 406, 407 and 408 (058); SEQ ID NOs: 185, 186, 187, 426, 427 and 428 (284); SEQ ID NOs: 709, 186, 187, 426, 427 and 428 (183); SEQ ID NOs: 709, 186, 187, 426, 427 and 428 (184); SEQ ID NOs: 189, 190, 191, 420, 421 and 422 (239); SEQ ID NOs: 189, 190, 191, 426, 427 and 428 (286) or SEQ ID NOs: 204, 205, 206, 420, 421 and 422 (247). In some embodiments, the bispecific antibody comprises a VHH1-CDR1, a VHH1-CDR2, and a VHH1-CDR3 and a VHH2-CDR1, a VHH2-CDR2, and a VHH2-CDR3, comprising the following amino acid sequences, respectively (E07-A05): SEQ ID NOs: 22, 23, 24, 291, 292 and 293 (877; according to Kabat); SEQ ID NOs: 22, 25, 26, 291, 294 and 295 (647; according to Kabat); SEQ ID NOs: 22, 27, 24, 291, 297 and 298 (195; according to Kabat); SEQ ID NOs: 77, 78, 79, 329, 330 and 331 (877; according to IMGT); SEQ ID NOs: 77, 80, 81, 329, 332 and 333 (647; according to IMGT); SEQ ID NOs: 77, 82, 79, 334, 335 and 336 (195; according to IMGT); SEQ ID NOs: 127, 128, 129, 369, 370 and 371 (877; according to Chothia); SEQ ID NOs: 127, 130, 131, 369, 372 and 373 (647; according to Chothia); SEQ ID NOs: 127, 130, 131, 374, 375 and 376 (195; according to Chothia); SEQ ID NOs: 174, 175, 176, 409, 410 and 411 (877; according to Honegger); SEQ ID NOs: 174, 177, 178, 409, 412 and 413 (647; according to Honegger) or SEQ ID NOs: 174, 179, 176, 414, 415 and 416 (195; according to Honegger). In some embodiments, the bispecific antibodies comprise a VHH1-CDR1, a VHH1-CDR2, and a VHH1-CDR3 and a VHH2-CDR1, a VHH2-CDR2, and a VHH2-CDR3, comprising the following amino acid sequences, respectively (F11-C04): SEQ ID NOs: 33, 34, 35, 296, 308 and 309 (284; according to Kabat); SEQ ID NOs: 33, 34, 35, 296, 308 and 309 (183; according to Kabat); SEQ ID NOs: 33, 34, 35, 296, 308 and 309 (184; according to Kabat); SEQ ID NOs: 88, 89, 90, 326, 327 and 328 (284; according to IMGT); SEQ ID NOs: 88, 89, 90, 326, 327 and 328 (183; according to IMGT); SEQ ID NOs: 88, 89, 90, 326, 327 and 328 (184; according to IMGT); SEQ ID NOs: 137, 138, 139, 386, 387 and 388 (284; according to Chothia); SEQ ID NOs: 137, 138, 139, 386, 387 and 388 (183; according to Chothia); SEQ ID NOs: 137, 138, 139, 386, 387 and 388 (184; according to Chothia); SEQ ID NOs: 185, 186, 187, 426, 427 and 428 (284; according to Honegger); SEQ ID NOs: 709, 186, 187, 426, 427 and 428 (183; according to Honegger) or; SEQ ID NOs: 709, 186, 187, 426, 427 and 428 (184; according to Honegger). In some embodiments, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in: SEQ ID NOs: 212 and 448 [A06con-A05con]; SEQ ID NOs: 212 and 449 [A06con-A05con]; SEQ ID NOs: 212 and 460 [A06con-C04con]; SEQ ID NOs: 233 and 448 [E07con-A05con]; SEQ ID NOs: 234 and 449 [E07con-A05con]; SEQ ID NOs: 233 and 460 [E07con-C04con]; SEQ ID NOs: 234 and 460 [E07con-C04con]; SEQ ID NOs: 710 and 448 [C09con-A05con]; SEQ ID NOs: 710 and 449; [C09con-A05con]; SEQ ID NOs: 710 and 460; [C09con-C04con]; SEQ ID NOs: 230 and 460 [G22con-C04con] or SEQ ID NOs: 249 and 460 [F11con-C04con]. In some embodiments, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in: SEQ ID NOs: 235 and 450 (877); SEQ ID NOs: 237 and 451 (647); SEQ ID NOs: 239 and 452 (195); SEQ ID NOs: 235 and 446 (048); SEQ ID NOs: 237 and 447 (049); SEQ ID NOs: 239 and 447 (050); SEQ ID NOs: 235 and 461 (274); SEQ ID NOs: 237 and 462 (771); SEQ ID NOs: 239 and 462 (772); SEQ ID NOs: 241 and 446 (054); SEQ ID NOs: 243 and 447 (755); SEQ ID NOs: 241 and 450 (883); SEQ ID NOs: 243 and 451 (784); SEQ ID NOs: 243 and 452 (785); SEQ ID NOs: 213 and 446 (046); SEQ ID NOs: 215 and 447 (747); SEQ ID NOs: 213 and 450 (875); SEQ ID NOs: 215 and 451 (776); SEQ ID NOs: 215 and 452 (777); SEQ ID NOs: 213 and 461 (272); SEQ ID NOs: 215 and 462 (773); SEQ ID NOs: 224 and 446 (052); SEQ ID NOs: 226 and 447 (753); SEQ ID NOs: 224 and 461 (278); SEQ ID NOs: 226 and 462 (769); SEQ ID NOs: 227 and 446 (047); SEQ ID NOs: 229 and 447 (748); SEQ ID NOs: 227 and 450 (876); SEQ ID NOs: 229 and 451 (778); SEQ ID NOs: 229 and 452 (779); SEQ ID NOs: 267 and 446 (062); SEQ ID NOs: 269 and 447 (763); SEQ ID NOs: 267 and 450 (891); SEQ ID NOs: 269 and 451 (780); SEQ ID NOs: 269 and 452 (781); SEQ ID NOs: 267 and 461 (290); SEQ ID NOs: 269 and 462 (791); SEQ ID NOs: 270 and 450 (892); SEQ ID NOs: 272 and 451 (793); SEQ ID NOs: 272 and 452 (794); SEQ ID NOs: 285 and 450 (894); SEQ ID NOs: 287 and 451 (795); SEQ ID NOs: 287 and 452 (796); SEQ ID NOs: 244 and 450 (884); SEQ ID NOs: 246 and 451 (786); SEQ ID NOs: 247 and 452 (787); SEQ ID NOs: 253 and 446 (058); SEQ ID NOs: 255 and 447 (759); SEQ ID NOs: 250 and 461 (284); SEQ ID NOs: 251 and 462 (183); SEQ ID NOs: 252 and 462 (184); SEQ ID NOs: 256 and 456 (239); SEQ ID NOs: 258 and 457 (740); SEQ ID NOs: 256 and 461 (286); SEQ ID NOs: 258 and 462 (788); SEQ ID NOs: 278 and 456 (247) SEQ ID NOs: 280 and 457 (749); SEQ ID NOs: 752 and 461; SEQ ID NOs: 752 and 462; SEQ ID NOs: 753 and 461; SEQ ID NOs: 753 and 462; SEQ ID NOs: 754 and 461; SEQ ID NOs: 754 and 462; SEQ ID NOs: 755 and 461; or SEQ ID NOs: 755 and 462. In some embodiments, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in: SEQ ID NOs: 236 and 450 (877); SEQ ID NOs: 238 and 451 (647); SEQ ID NOs: 240 and 452 (195); SEQ ID NOs: 236 and 446 (048); SEQ ID NOs: 238 and 447 (049); SEQ ID NOs: 240 and 447 (050); SEQ ID NOs: 236 and 461 (274); SEQ ID NOs: 238 and 462 (771); SEQ ID NOs: 240 and 462 (772); SEQ ID NOs: 242 and 446 (054); SEQ ID NOs: 243 and 447 (755); SEQ ID NOs: 242 and 450 (883); SEQ ID NOs: 243 and 451 (784); SEQ ID NOs: 243 and 452 (785); SEQ ID NOs: 214 and 446 (046); SEQ ID NOs: 215 and 447 (747); SEQ ID NOs: 214 and 450 (875); SEQ ID NOs: 215 and 451 (776); SEQ ID NOs: 215 and 452 (777); SEQ ID NOs: 214 and 461 (272); SEQ ID NOs: 215 and 462 (773); SEQ ID NOs: 225 and 446 (052); SEQ ID NOs: 226 and 447 (753); SEQ ID NOs: 225 and 461 (278); SEQ ID NOs: 226 and 462 (769); SEQ ID NOs: 228 and 446 (047); SEQ ID NOs: 229 and 447 (748); SEQ ID NOs: 228 and 450 (876); SEQ ID NOs: 229 and 451 (778); SEQ ID NOs: 229 and 452 (779); SEQ ID NOs: 268 and 446 (062); SEQ ID NOs: 269 and 447 (763); SEQ ID NOs: 268 and 450 (891); SEQ ID NOs: 269 and 451 (780); SEQ ID NOs: 269 and 452 (781); SEQ ID NOs: 268 and 461 (290); SEQ ID NOs: 269 and 462 (791); SEQ ID NOs: 271 and 450 (892); SEQ ID NOs: 273 and 451 (793); SEQ ID NOs: 274 and 452 (794); SEQ ID NOs: 286 and 450 (894); SEQ ID NOs: 287 and 451 (795); SEQ ID NOs: 287 and 452 (796); SEQ ID NOs: 245 and 450 (884); SEQ ID NOs: 246 and 451 (786); SEQ ID NOs: 246 and 452 (787); SEQ ID NOs: 254 and 446 (058); SEQ ID NOs: 255 and 447 (759); SEQ ID NOs: 250 and 461 (284); SEQ ID NOs: 251 and 462 (183); SEQ ID NOs: 252 and 462 (184); SEQ ID NOs: 752 and 461; SEQ ID NOs: 752 and 462; SEQ ID NOs: 753 and 461; SEQ ID NOs: 753 and 462; SEQ ID NOs: 754 and 461; SEQ ID NOs: 754 and 462; SEQ ID NOs: 755 and 461; or SEQ ID NOs: 755 and 462; SEQ ID NOs: 257 and 456 (239); SEQ ID NOs: 259 and 457 (740); SEQ ID NOs: 257 and 461 (286); SEQ ID NOs: 259 and 462 (788); SEQ ID NOs: 279 and 456 (247) or SEQ ID NOs: 280 and 457 (749). In some embodiments, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in: SEQ ID NOs: 233 and 448 [E07con-A05con]; SEQ ID NOs: 234 and 449 [E07con-A05con]; SEQ ID NOs: 235 and 450 (877); SEQ ID NOs: 237 and 451 (647); SEQ ID NOs: 239 and 452 (195); SEQ ID NOs: 236 and 450 (877); SEQ ID NOs: 238 and 451 (647); SEQ ID NOs: 240 and 452 (195); SEQ ID NOs: 724 and 450 (E07-A05); SEQ ID NOs: 724 and 451 (E07-A05); SEQ ID NOs: 724 and 452 (E07-A05); SEQ ID NOs: 725 and 450 (E07-A05); SEQ ID NOs: 725 and 451 (E07-A05) or SEQ ID NOs: 725 and 452 (E07-A05). In some embodiments, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in: SEQ ID NOs: 249 and 460 [F11con-C04con]; SEQ ID NOs: 250 and 461 (284); SEQ ID NOs: 251 and 462 (183); SEQ ID NOs: 252 and 462 (184); SEQ ID NOs: 250 and 461 (284); SEQ ID NOs: 251 and 462 (183); SEQ ID NOs: 252 and 462 (184); SEQ ID NOs: 752 and 461; SEQ ID NOs: 752 and 462; SEQ ID NOs: 753 and 461; SEQ ID NOs: 753 and 462; SEQ ID NOs: 754 and 461; SEQ ID NOs: 754 and 462; SEQ ID NOs: 755 and 461; or SEQ ID NOs: 755 and 462 (F11-C04). In some embodiments, the bispecifc antibody comprises in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 475-476 and 573. In some embodiments, the bispecifc antibody comprises in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 574, 576, 578, 580, 582, 584, 586, 588 and 590. In some embodiments, the bispecifc antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 575, 577, 579, 581, 583, 585, 587, 589, 591, 737-751 and 757-758. In some embodiments, the bispecifc antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 475-482 and 737-751 (E07-A05). In some embodiments, the bispecifc antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 573-579 and 757-758 (F11-C04). In some embodiments of the bispecific antibody, the first VHH that specifically binds to IL 10Rα binds to both human IL 10Rα and cynomolgus IL 10Rα. In some embodiments of the bispecific antibody, the second VHH that specifically binds to IL 10Rβ binds to both human IL 10Rβ and cynomolgus IL 10Rβ. In some embodiments of the bispecific antibody, the first VHH that specifically binds to IL 10Rα binds to both human IL 10Rα and cynomolgus IL 10Rα and wherein the second VHH that specifically binds to IL 10Rβ binds to both human IL 10Rβ and cynomolgus IL 10Rβ. In some embodiments, the bispecifc antibody comprises a first fragment crystallizable (Fc) domain and a second Fc domain. In some embodiments, the first Fc domain and the second Fc domain are a human IgG1 or a human IgG4. In some embodiments, one or both of the first Fc domain and the second Fc domain comprises one or more amino acid substitutions that reduces or inhibits or eliminates antibody-dependent cell-mediated cytotoxicity (ADCC) effector function. In some embodiments, one or both of the first Fc domain and the second Fc domain comprises effector function reducing amino acid substitutions at Fe positions (EU numbering) selected from: alanine at position 234 and alanine at position 235 (234A and 235A); glutamic acid at position 235 (235E); proline at position 228 and glutamic acid at position 235 (228P, 235E); alanine at position 237 (237A); alanine at position 234 and glutamic acid at position 235 (234A and 235E); phenylalanine at position 234, glutamic acid at position 235 and alanine at position 265 (234F, 235E and 265A); phenylalanine at position 234, glutamic acid at position 235 and alanine at position 236 (234F, 235E and 236A); phenylalanine at position 234, glutamic acid at position 235 and arginine at position 236 (234F, 235E and 236R); serine at position 234, threonine at position 235 and arginine at position 236 (234S, 235T and 236R); alanine at position 234, alanine at position 235 and glycine at position 329 (234A, 235A and 329G); alanine at position 234, alanine at position 235 and alanine at position 237 (234A, 235A and 237A); proline at position 228 and glutamic acid at position 235 (228P and 235E); proline at position 228, glutamic acid in position 235 and glycine at position 329 (228P, 235E and 329G); phenylalanine at position 234, glutamic acid at position 235 and serine at position 331 (234F, 235E and 331S); phenylalanine at position 234, glutamine at position 235 and glutamine at position 322 (234F, 235Q and 322Q); alanine at position 233, arginine at position 234 and arginine at position 235 (233A, 234R and 235R); alanine at position 233, aspartic acid at position 234 and glutamic acid at position 235 (233A, 234D and 235E); lysine at position 233, arginine at position 234 and arginine at position 235 (233K, 234R and 235R); alanine at position 234, alanine at position 235, alanine at position 237, serine at position 238, alanine at position 268, serine at position 330 and serine at position 331 (234A, 235A, 237A, 238S, 268A, 330S and 3315); and proline at position 228, alanine at position 234, alanine at position 235, alanine at position 237 and serine at position 238 (228P, 234A, 235A, 237A and 238S). In some embodiments, one or both of the first Fc domain and the second Fc domain comprises effector function reducing amino acid substitutions at Fe positions (EU numbering) alanine at position 234, alanine at position 235 and alanine at position 237 (234A, 235A and 237A). In some embodiments, one or both of the first Fc domain and the second Fc domain comprises one or more amino acid substitutions that extend serum half-life. In some embodiments, one or both of the first Fc domain and the second Fc domain comprises serum half-life extending amino acid substitutions at Fe positions (EU numbering) selected from: tyrosine at position 252, threonine at position 254 and glutamic acid at position 256 (252Y, 254T and 256E); leucine at position 428 (428L); glutamine at position 250 and leucine at position 428 (250Q and 428L); leucine at position 428 and serine at position 434 (428L and 434S); leucine at position 428 and alanine at position 434 (428L and 434A); arginine at position 311 and leucine at position 428 (311R and 428L); glycine at position 309 and leucine at position 428 (309G and 428L); glutamine at position 307, valine at position 311 and valine at position 378 (307Q, 311V and 378V); and aspartic acid at position 256, aspartic acid at position 286, arginine at position 307, valine at position 311 and valine at position 378 (256D, 286D, 307R, 311V and 378V). In some embodiments, the bispecific antibody comprises at least one polypeptide comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 592-593 and 690. In some embodiments, the bispecific antibody comprises at least one polypeptide comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 691, 693, 695, 697, 699, 701, 703, 705, and 707. In some embodiments, the bispecific antibody comprises at least one polypeptide comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 692, 694, 696, 698, 700, 702, 704, 706 and 708. In some embodiments, the bispecific antibody comprises at least one polypeptide comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 592-599 (E07-A05). In some embodiments, the bispecific antibody comprises at least one polypeptide comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 690-696 (F11-C04). In some embodiments, the first VHH that specifically binds to IL-10Rα and the second VHH that specifically binds to IL-10Rβ are both humanized. In some embodiments, the bispecific antibody has a serum half-life in a human of at least 3 days, e.g., at least 4 days, at least 5 days, at least 6 days, at least 7 days, at least 8 days, at least 9 days, at least 10 days, at least 11 days, at least 12 days, or longer.

Further provided are means for agonizing an IL-10 receptor that selectively suppresses activation of myeloid cells and induces minimal or no direct activation of T cells or B cells. Also provided are means for agonizing an IL-10 receptor that predominantly suppresses activation of myeloid cells and induces minimal or no direct activation of T cells or B cells. In some embodiments, the means induces a maximum effective (Emax) activation of T cells or B cells that is 15% or less of wild-type IL-10. Also provided are means for agonizing an IL-10 receptor that induces a maximum effective (Emax) activation of T cells or B cells that is 15% or less of wild-type IL-10. Also provided are means for agonizing an IL-10 receptor that suppresses activation of myeloid cells with a half maximal effective concentration (EC₅₀) less than 10 pM and activating T cells and B cells below a predetermined threshold level at a concentration above 1000 nM. In some embodiments, the means agonizes the IL-10 receptor in a tyrosine kinase 2 (TYK2; NCBI Gene ID: 7297) dependent manner. In some embodiments, the activation of T cells is measured by at least one of Granzyme B secretion and interferon-γ (IFN-γ) production. In some embodiments, the means does not induce detectable granzyme B the secretion from CD8+ T cells. In some embodiments, the means induces interferon-γ (IFN-γ) from CD8+ T cells less than 15% the level of IFN-γ induced by wild-type IL-10. In some embodiments, the myeloid cells are selected from macrophages, monocytes and dendritic cells. In some embodiments, the means induces minimal (e.g., less than 25% in comparison to wild-type IL-10) or no inhibition of IL-10Rβ dimerization with other IL-10 family cytokine receptor subunits, e.g., IL-10Rβ dimerization with IL-20Rα, IL-22R, IL-28R or IL-29R. In some embodiments, the means induces minimal (e.g., less than 25% in comparison to wild-type IL-10) or no inhibition of intracellular signaling of other IL-10Rβ-binding cytokines, e.g., minimal or no inhibition of IL-22, IL-26, IL-28A, IL-28B, or IL-29 intracellular signaling. In some embodiments, the means binds to IL-10Rβ with an equilibrium dissociation constant (K_{D}) that is at least 5-fold greater, e.g., at least 7-fold greater, e.g., at least 10-fold, 15-fold, 20-fold, 25-fold greater, or more, than the K_{D} of binding to IL-10Rα, e.g., wherein the binding affinity is determined by surface plasmon resonance (SPR). In some embodiments, the means binds to IL-10Rα with an equilibrium dissociation constant (K_{D}) of lower than 2 nM and binds to IL-10Rβ with a K_{D} of greater than 10 nM, e.g., greater than 50 nM, e.g., wherein the binding affinity is determined by surface plasmon resonance (SPR). In some embodiments, the means binds to IL-10Rα contact amino acid residues W33, E35, E37, H40, 142, H44, T46, L79, 580, T124, A170, R172, F178, F180, H182, Q204, K215, M217, W218, K220 and E221, wherein the amino acid residue positions are with respect to SEQ ID NO: 711 and binds to IL-10Rβ contact amino acid residues S78, S80, K81, Y82, V108, N138, E139, Y140, E141, T142, W143, N147, V148 and N150, wherein the amino acid residue positions are with respect to SEQ ID NO: 712. In some embodiments, the means binds to IL-10Rα contact amino acid residues 1169, I171, K173, F178, F180, T181, H182, K183, K186, F190, L192, L193, S195, E197, and F201, wherein the amino acid residue positions are with respect to SEQ ID NO: 711 and binds to IL-10Rβ contact amino acid residues N26, R28, N30, V32, Q39, E41, T72, D84, 1103, T104, D110, I112, P115, Q157, Q190, R192, F194, N199, A201, E203, W204, and E206, wherein the amino acid residue positions are with respect to SEQ ID NO: 712.

Further provided is a polynucleotide that encode the sdAb that binds to IL-10Rα; the sdAb that binds to IL-10Rβ or the bispecific antibody, as described above and herein. In some embodiments, the polynucleotide is DNA or RNA, e.g., cDNA or mRNA. Further provided is an expression cassette comprising one or more regulatory sequences operably linked to the polynucleotide, as described above and herein. In some embodiments, the one or more regulatory sequences comprise a constitutive promoter. Further provided in an expression vector comprising one or more regulatory sequences operably linked to the polynucleotide or polynucleotides or the expression cassette or expression cassettes as described above and herein. In some embodiments, the expression vector comprises a plasmid vector or a viral vector.

Further provided is a method of producing an IL-10R binding molecule, as described herein. In some embodiments, the method comprises: (a) culturing a cell or population of cells as described herein transformed with the polynucleotide as described herein, or the expression cassette as described herein in a cell culture under conditions sufficient to express the IL-10R binding molecule; and (b) isolating or purifying the antigen binding molecules from the cell culture. Further provided is cell or population of cells, comprising the polynucleotide or polynucleotides, the expression cassette, or the expression vector as described above and herein. In some embodiments, the cell or population of cells comprises a eukaryotic cell. In some embodiments, the cell or population of cells comprises a mammalian cell, an insect cell, a plant cell or a yeast cell. In some embodiments, the mammalian cell is a Chinese Hamster Ovary (CHO) cell.

Further provided is a method of preventing or treating an inflammatory disease in a subject in need thereof, comprising administering to the subject an effective amount of a bispecific antibody as described above and herein. Also provided is a method of reducing the severity of or mitigating on or more symptoms of an inflammatory disease in a subject in need thereof, comprising administering to the subject an effective amount of a bispecific antibody as described above and herein. In some embodiments, the inflammatory disease is mediated by an IL-10 deficiency or deficient signaling through the IL-10R. In some embodiments, the inflammatory disease is an autoimmune disease. In some embodiments, the inflammatory disease is an inflammatory bowel disease. In some embodiments, the inflammatory bowel disease is selected from ulcerative colitis, Crohn's disease, mucosal proctocolitis and celiac disease. In some embodiments, the inflammatory disease is an inflammatory dermatitis disease. In some embodiments, the inflammatory dermatitis disease is selected from atopic dermatitis (AD), hidradenitis suppurativa (HS), and psoriasis. In some embodiments, the inflammatory disease is selected from ulcerative colitis, Crohn's disease, mucosal proctocolitis and celiac disease, rheumatoid arthritis (RA), juvenile idiopathic arthritis, atopic dermatitis (AD), hidradenitis suppurativa (HS), psoriasis, systemic lupus erythematosus (SLE), Behget's disease, type 1 diabetes, primary sclerosing cholangitis, ankylosing spondylitis, and multiple sclerosis. In some embodiments, the inflammatory disease is acute or chronic. In some embodiments, the bispecific antibody is administered subcutaneously, intravenously or orally. In some embodiments, the bispecific antibody is administered via a device. In some embodiments, the device is configured for sustained delivery. In some embodiments, the device is configured for self-administration. In some embodiments, the device is a prefilled syringe. In some embodiments, the device is an ingestible device configured for local delivery to the intestine. In some embodiments, the bispecific antibody is combined or co-administered with one or more additional agents. In some embodiments, the one or more additional agents are selected from infliximab, adalimumab, golimumab, vedolizumab, tofacitinib, ustekinumab, natalizumab, mesalamine, diazo-bonded 5-ASA, sulfasalazine, balsalazide, olsalazine, a corticosteroid (e.g., budesonide, hydrocortisone, methylprednisolone, prednisone); an immunosuppressant (e.g., azathioprine, 6-mercaptopurine, cyclosporine, methotrexate), and combinations thereof. In some embodiments, the one or more additional agents are selected from a PPARδ inhibitor, an IRAK4 inhibitor, a TPL2 inhibitor, an α4β7 inhibitor, a BTLA agonist, a PD-1 agonist, a CD200R agonist, or a FXR agonist. In some embodiments, the one or more additional agents are selected from seladelpar, edecesertib, tilpisertib fosmecarbil, GS-1427, GS 0272, GS-0151, GS-6791 and cilofexor.

Further provided is a kit comprising one or more unitary doses of a bispecific antibody as described above and herein. In some embodiments, the one or more unitary doses are the same or different. In some embodiments, the one or more unitary doses are in one or more ampoules, vials, pre-loaded syringes or delivery devices. In some embodiments, the bispecific antibody is combined or co-administered with one or more additional agents. In some embodiments, the one or more additional agents are selected from infliximab, adalimumab, golimumab, vedolizumab, tofacitinib, ustekinumab, natalizumab, mesalamine, diazo-bonded 5-ASA, sulfasalazine, balsalazide, olsalazine, a corticosteroid (e.g., budesonide, hydrocortisone, methylprednisolone, prednisone); an immunosuppressant (e.g., azathioprine, 6-mercaptopurine, cyclosporine, methotrexate), and combinations thereof. In some embodiments, the one or more additional agents are selected from a PPARδ inhibitor, an IRAK4 inhibitor, a TPL2 inhibitor, an α4β7 inhibitor, a BTLA agonist, a PD-1 agonist, a CD200R agonist, or a FXR agonist. In some embodiments, the one or more additional agents are selected from seladelpar, edecesertib, tilpisertib fosmecarbil, GS-1427, GS 0272, GS-0151, GS-6791 and cilofexor.

### BRIEF DESCRIPTION OF THE DRAWINGS

FIG. 1 illustrates potency of IL-10R agonistic potency, as determined by Eₘₐₓ, of αIL-10Rα-IL-10Rβ tandem VHH1-VHH2 bispecific molecules in a HEK293T IL-10R-pSTAT3 luciferase reporter assay. Percentage of Eₘₐₓ was normalized by recombinant IL-10.

FIGS. 2A-2D illustrate cryo-EM structure of IL-10Rα+IL-10Rβ+E07-A05 tandem VHH1-VHH2. FIG. 2A depicts the overall structure of IL-10Rα+IL-10Rβ+E07-A05 tandem VHH1-VHH2 complex. FIGS. 2B and 2C depict the E07 epitope contact residues. FIG. 2D depicts the A05 epitope contact residues. Underlined amino acid positions are residues on the VHH, while amino acid positions without an underline are residues on IL-10Rα or IL-10Rβ.

FIGS. 3A-3D illustrate cryo-EM structure of IL-10Rα+IL-10Rβ+F11-C04 tandem VHH1-VHH2. FIG. 3A depicts the overall structure of IL-10Rα+IL-10Rβ+F11-C04 tandem VHH1-VHH2 complex. FIG. 3B depicts the F11 epitope contact residues. FIGS. 3C and 3D depicts the C04 epitope contact residues. Underlined amino acid positions are residues on the VHH, while amino acid positions without an underline are residues on IL-10Rα or IL-10Rβ.

FIG. 4 illustrates cryo-EM structure of IL-10Rβ+VHH A05+VHH C04.

FIG. 5 illustrates tandem VHH1-VHH2 bispecific molecules profiled in a Colo205 IL-22R-pSTAT3 luciferase reporter assay.

FIG. 6 illustrates tandem VHH1-VHH2 bispecific molecules profiled in a HEK293T IL-29 reporter assay.

FIG. 7 illustrates data from the dose-response curves for phospho-STAT3 in human whole blood stimulated with IL-10-Fc or our VHH1-VHH2 bispecific molecules 195 and 184, analyzed by flow cytometry. Data are shown as phospho-STAT3 MFI (N=2).

FIGS. 8A-8B illustrate that VHH1-VHH2 bispecific molecules 195 and 184 inhibit lipopolysaccharide (LPS) induced IL-6 secretion from peripheral blood mononuclear cells (PBMC).

FIG. 9 illustrates that VHH1-VHH2 bispecific molecules 195 and 184 do not significantly induce IFN-γ secretion from primary CD8+ T cells.

FIG. 10 illustrates that comparator anti-IL-10Rαα VHH1-VHH2-Fc bispecific molecule (C1) inhibits TNFα release by LPS.

FIG. 11 illustrates that comparator anti-IL-10Rαβ VHH1-VHH2-Fc bispecific molecule (C1) induces STAT3 phosphorylation in monocytes, CD4+ T cells, CD8+ T cells, and B cells.

FIG. 12 illustrates that comparator anti-IL-10Rαβ VHH1-VHH2-Fc bispecific molecule (C1) promotes T cell production of IFN-γ and granzyme B.

FIGS. 13A-13E illustrate dose-response curves for phospho-STAT3 in monocytes (FIG. 13A), CD8+ T cells (FIG. 13B), CD4+ T cells (FIG. 13C), NK cells (FIG. 13D) and B cells (FIG. 13E) from human whole blood stimulated with IL-10-Fc, VHH1-VHH2 bispecific molecules 195 and 184, and comparator VHH1-VHH2 bispecific molecules, analyzed by flow cytometry. Data are expressed as phospho-STAT3 MFI (N=3).

FIG. 14 illustrates the VHH1-VHH2-His monovalent version of 302 (monovalent 195) and 196 (monovalent 184) induces less IFN-γ secretion from primary CD8+ T cells in comparison to VHH1-VHH2-His monovalent versions of comparator molecules.

FIG. 15 illustrates VHH1-VHH2-Fc version of 195 and 184 induce less IFN-γ secretion from primary CD8+ T cells in comparison to VHH1-VHH2-Fc versions of comparator molecules.

FIG. 16 illustrates that VHH1-VHH2 bispecific molecules 195 and 184 inhibit monocyte differentiation into dendritic cells (defined as CD1a+CD14− cells). IL-10-Fc was included as a positive control for comparison. Each dot represents an individual human donor.

FIG. 17 illustrates that VHH1-VHH2 bispecific molecules 195 and 184 inhibit lipopolysaccharide (LPS)-induced dendritic cell maturation, measured by the expression of DC activation marker CD83 and co-stimulatory marker CD86. Activated DCs were quantified as CD83+CD86+ cells. IL-10-Fc was included as a positive control for comparison. Each dot represents an individual human donor.

FIG. 18 illustrates that VHH1-VHH2 bispecific molecules 195 and 184 inhibit zymosan induced dendritic cell secretion of TNFα and IL-23. IL-10-Fc was included as a positive control for comparison. Each dot represents an individual human donor.

FIG. 19 illustrates that VHH1-VHH2 bispecific molecules 195 and 184 inhibit dendritic cell (DC) antigen presentation and tetramer-positive T-cell expansion. IL-10-Fc was included for comparison. Each dot represents an individual human donor.

FIGS. 20A-20D illustrate that both IL-10-Fc and VHH1-VHH2 bispecific molecules 195 and 184 demonstrate potent inhibition of proinflammatory cytokines IL-17 (20A), IL-6 (20B), IL-12p70 (20C) and TNFα (20D) during dose titration.

FIGS. 21A-21C illustrate data from an efficacy test of VHH1-VHH2 bispecific molecule 877 in a dextran sulfate sodium (DSS)-induced colitis model. FIG. 21A shows the ratio of colon length and colon weight in different treatment groups. FIG. 21B shows inflammatory cytokine gene expression level in colon tissue using quantitative PCR. FIG. 21C shows neutrophil frequency change in spleen using flow cytometry and Csf3r (neutrophil marker) gene expression change in colon tissue using quantitative PCR.

FIG. 22 illustrates results of an assay measuring pre-existing anti-drug antibodies (ADA) against VHH1-VHH2 bispecific molecules 184, 183, 647 and 195 and positive control caplacizumab-Fc. RLU=relative light units.

FIG. 23 illustrates the results of an assay measuring the T-cell immunogenicity elicited by VHH1-VHH2 bispecific molecules 195, 647, 183 and 184 in human PBMCs. Avastin (bevacizumab) was used as negative control and both bococizumab and keyhole limpet hemocyanin (KLH) protein were used as positive controls in this assay.

FIG. 24 illustrates pharmacokinetic profiles for anti-IL-10Rαβ agonist VHH1-VHH2-Fc molecules 195 (circle) and 184 (square) after 100 mg/kg IV bolus infusion in naïve cynomolgus macaques. Each symbol is the measured mean±standard deviation (SD) plasma concentration.

FIG. 25 illustrates STAT3 phosphorylation (pSTAT3) of human macrophages (white/no fill); TYK2 CRISPR knock-out (KO) human macrophages (dark gray); and control (non-targeted RNA) CRISPR KO human macrophages (light gray). Wild-type IL-10 or VHH1-VHH2 bispecific molecule 184 were added to indicated human macrophages. pSTAT3 was quantified by flow cytometry analysis.

FIG. 26 illustrates Western blotting of phosphorylated TYK2 (pTYK2), total TYK2, phosphorylated JAK1 (pJAK1), total JAK1, phosphorylated STAT3 (pSTAT3), and GADPH at different time points after addition of wild-type IL-10 or VHH1-VHH2 bispecific molecule 877 at indicated time points.

FIGS. 27A-27C. PBMCs from two donors were stimulated with LPS in the presence of the indicated molecules and a TYK2 small-molecule inhibitor. Cell culture supernatants were analyzed for levels of cytokines, including TNF (FIG. 27A), IL-6 (FIG. 27B), and IL-1β (FIG. 27C).

FIG. 28 illustrates comparative potency analysis of anti-IL-10Rαβ agonist VHH1-VHH2-Fc molecules and comparator molecules in HEK293 IL-10R pSTAT3 luciferase reporter cells.

FIG. 29 illustrates confirmation of TYK2 knockout in HEK293 TYK2 KO IL-10R pSTAT3 luciferase reporter cells.

FIG. 30 illustrates comparative potency analysis of anti-IL-10Rαβ agonist VHH1-VHH2-Fc molecules and comparator molecules in HEK293 TYK2 KO IL-10R pSTAT3 luciferase reporter cells.

FIG. 31 illustrates the effect of IL-10-Fc in the collagen induced arthritis (CIA) mouse model. Clinical arthritis scores from indicated group treated with isotype control, IL-10-Fc, or anti-IL-17A antibody.

FIG. 32 illustrates the effect of IL-TO-Fc in the mannan induced inflammatory dermatitis mouse model. Clinical dermatitis scores from indicated group treated with isotype control, IL-10-Fc, or anti-IL-17A antibody.

### DETAILED DESCRIPTION

### 1. Introduction

In this study, we employed extensive protein engineering on single-domain antibodies to generate potent anti-IL-10 receptor alpha subunit and anti-IL-10 beta subunit (IL-10Rαβ) agonists that selectively target myeloid cells. These IL-10Rαβ agonists exhibit strong myeloid cell specific immunosuppressive functions without activating T-cells, B-cells, or NK cells.

### 2. IL-10Rα Single Immunoglobulin Variable Domain (VHH) Binders

Provided are single immunoglobulin variable domain (VHH) binders that specifically bind to the extracellular domain of interleukin-10 receptor (IL-10R) alpha subunit (IL-10Rα). Illustrative IL-10Rα single immunoglobulin variable domain (VHH) binders are provided in Tables A1-A4 and B, below. The IL-10Rα single immunoglobulin variable domain (VHH) binders can be humanized and incorporated into antibodies, including multispecific antibodies, e.g., bispecific antibodies, e.g., comprising a human Fc domain, e.g., a human IgG1 or a human IgG4. The IL-10Rα single immunoglobulin variable domain (VHH) binders described herein are suitable to use with an antigen binding domain that specifically binds to the extracellular domain of interleukin-10 receptor (IL-10R) beta subunit (IL 10Rβ), including the IL-10Rβ single immunoglobulin variable domain (VHH) binders described herein.

With regard to the binding of an antibody or antigen-binding fragment thereof to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. An antibody or antigen-binding fragment thereof that “specifically binds to” or is “specific for” a particular polypeptide or an epitope on a particular polypeptide is one that binds to that particular polypeptide or epitope on a particular polypeptide without substantially binding to any other polypeptide or polypeptide epitope. Affinities of antibodies can be readily determined using conventional techniques, for example, those described by Scatchard et al. (Ann. N. Y. Acad. Sci. USA 51: 660 (1949), ELISA assays, biolayer interferometry (BLI) assays, and surface plasmon resonance (SPR) assays). Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

In some embodiments, the VHH binds to IL 10Rα with an equilibrium dissociation constant (K_{D}) of lower than about 5 nM, e.g., lower than about 4 nM, lower than about 3 nM or lower than about 2 nM.

In some embodiments, the VHH comprises a complementarity determining region (CDR) 1, a VHH-CDR2, and a VHH-CDR3 comprising the following amino acid sequences, according to Kabat, respectively:
1) SEQ ID NOs: 1, 2 and 3; (A01)
2) SEQ ID NOs: 4, 5 and 6; (A06)
3) SEQ ID NOs: 7, 8 and 9: (A09)
4) SEQ ID NOs: 10, 11 and 12; (B07)
5) SEQ ID NOs: 4, 13 and 14; (B08)
6) SEQ ID NOs: 4, 15 and 16; (C09)
7) SEQ ID NOs: 4, 17 and 18; (D08)
8) SEQ ID NOs: 19, 20 and 21; (D14)
9) SEQ ID NOs: 22, 23 and 24; (E07)
10) SEQ ID NOs: 22, 25 and 27; (huE07)
11) SEQ ID NOs: 22, 27 and 24; (huE07)
12) SEQ ID NOs: 4, 28 and 29; (E15)
13) SEQ ID NOs: 4, 30 and 31; (F09)
14) SEQ ID NOs: 22, 23 and 32; (F10)
15) SEQ ID NOs: 33, 34 and 35; (F11)
16) SEQ ID NOs: 4, 36 and 29; (F21)
17) SEQ ID NOs: 19, 37 and 38; (G22)
18) SEQ ID NOs: 39, 40 and 41; (G23)
19) SEQ ID NOs: 42, 43 and 44; (H22)
20) SEQ ID NOs: 45, 46 and 47; (I04)
21) SEQ ID NOs: 4, 48 and 6; (J13)
22) SEQ ID NOs: 4, 49 and 50; (J24)
23) SEQ ID NOs: 19, 51 and 52; (L05)
24) SEQ ID NOs: 19, 37 and 53; (L11) or
25) SEQ ID NOs: 54, 55 and 24 (N16).

In some embodiments, the VHH comprises a complementarity determining region (CDR) 1, a VHH-CDR2, and a VHH-CDR3 comprising the following amino acid sequences, according to IMGT, respectively:
1) SEQ ID NOs: 56, 57 and 58; (A01)
2) SEQ ID NOs: 59, 60 and 61; (A06)
3) SEQ ID NOs: 62, 63 and 64; (A09)
4) SEQ ID NOs: 65, 66 and 67; (B07)
5) SEQ ID NOs: 59, 68 and 69; (B08)
6) SEQ ID NOs: 59, 70 and 71; (C09)
7) SEQ ID NOs: 59, 72 and 73; (D08)
8) SEQ ID NOs: 74, 75 and 76; (D14)
9) SEQ ID NOs: 77, 78 and 79; (E07)
10) SEQ ID NOs: 77, 80 and 81; (huE07)
11) SEQ ID NOs: 77, 82 and 79; (huE07)
12) SEQ ID NOs: 59, 83 and 84; (E15)
13) SEQ ID NOs: 59, 85 and 86; (F09)
14) SEQ ID NOs: 77, 78 and 87; (F10)
15) SEQ ID NOs: 88, 89 and 90; (F11)
16) SEQ ID NOs: 59, 91 and 84; (F21)
17) SEQ ID NOs: 92, 75 and 93; (G22)
18) SEQ ID NOs: 94, 89 and 95; (G23)
19) SEQ ID NOs: 96, 89 and 97; (H22)
20) SEQ ID NOs: 98, 99 and 100; (I04)
21) SEQ ID NOs: 59, 101 and 61; (J13)
22) SEQ ID NOs: 59, 102 and 103; (J24)
23) SEQ ID NOs: 74, 75 and 104; (L05)
24) SEQ ID NOs: 74, 75 and 105; (L11) or
25) SEQ ID NOs: 106, 107 and 79 (N16).

In some embodiments, the VHH comprises a complementarity determining region (CDR) 1, a VHH-CDR2, and a VHH-CDR3 comprising the following amino acid sequences, according to Chothia, respectively:
1) SEQ ID NOs: 108, 109 and 110; (A01)
2) SEQ ID NOs: 111, 112 and 113; (A06)
3) SEQ ID NOs: 114,115 and 116; (A09)
4) SEQ ID NOs: 117,118 and 119; (B07)
5) SEQ ID NOs: 111, 112 and 120; (B08)
6) SEQ ID NOs: 111, 121 and 122; (C09)
7) SEQ ID NOs: 111, 121 and 123; (D08)
8) SEQ ID NOs: 124, 125 and 126; (D14)
9) SEQ ID NOs: 127, 128 and 129; (E07)
10) SEQ ID NOs: 127, 130 and 131; (huE07)
11) SEQ ID NOs: 111, 132 and 133; (E15)
12) SEQ ID NOs: 111, 134 and 135; (F09)
13) SEQ ID NOs: 127, 128 and 136; (F10)
14) SEQ ID NOs: 137, 138 and 139; (F11)
15) SEQ ID NOs: 111, 132 and 133; (F21)
16) SEQ ID NOs: 140, 125 and 141; (G22)
17) SEQ ID NOs: 142, 138 and 143; (G23)
18) SEQ ID NOs: 124, 138 and 144; (H22)
19) SEQ ID NOs: 145, 146 and 147; (I04)
20) SEQ ID NOs: 111, 112 and 113; (J13))
21) SEQ ID NOs: 111, 148 and 149; (J24)
22) SEQ ID NOs: 124, 125 and 150; (L05)
23) SEQ ID NOs: 124, 125 and 151; (L11) or
24) SEQ ID NOs: 152, 121 and 129 (N16).

In some embodiments, the VHH comprises a complementarity determining region (CDR) 1, a VHH-CDR2, and a VHH-CDR3 comprising the following amino acid sequences, according to Honegger, respectively:
1) SEQ ID NOs: 153, 154 and 155; (A01)
2) SEQ ID NOs: 156, 157 and 158; (A06)
3) SEQ ID NOs: 159, 160 and 161; (A09)
4) SEQ ID NOs: 162, 163 and 164; (B07)
5) SEQ ID NOs: 156, 165 and 166; (B08)
6) SEQ ID NOs: 156, 167 and 168; (C09)
7) SEQ ID NOs: 156, 169 and 170; (D08)
8) SEQ ID NOs: 171, 172 and 173; (D14)
9) SEQ ID NOs: 174, 175 and 176; (E07)
10) SEQ ID NOs: 174, 177 and 178; (huE07)
11) SEQ ID NOs: 174, 179 and 176; (huE07)
12) SEQ ID NOs: 156, 180 and 181; (E15)
13) SEQ ID NOs: 156, 182 and 183; (F09)
14) SEQ ID NOs: 174, 175 and 184; (F10)
15) SEQ ID NOs: 185, 186 and 187; (F11)
16) SEQ ID NOs: 709, 186 and 187; (huF11)
17) SEQ ID NOs: 156, 188 and 181; (F21)
18) SEQ ID NOs: 189, 190 and 191; (G22)
19) SEQ ID NOs: 192, 193 and 194; (G23)
20) SEQ ID NOs: 195, 196 and 197; (H22)
21) SEQ ID NOs: 198, 199 and 200; (I04)
22) SEQ ID NOs: 156, 201 and 158; (J13)
23) SEQ ID NOs: 156, 202 and 203; (J24)
24) SEQ ID NOs: 204, 205 and 206; (L05)
25) SEQ ID NOs: 204, 190 and 207; (L11) or
26) SEQ ID NOs: 208, 209 and 176 (N16).

In some embodiments, the VHH comprises the amino acid sequence, or an amino acid sequence of any one of SEQ ID NOs: 212, 230, 233, 234, 249, 710 and 724-725, or that is at least 80%, at least 85%, at least 90%, at least 91%, at least 92%, at least 93%, at least 94%, at least 95%, at least 96%, at least 97%, at least 98%, or at least 99% identical, to one of the amino acid sequences of any one of SEQ ID NOs: 212, 230, 233, 234, 249, 710 and 724-725. In some embodiments, the VHH comprises the amino acid sequence of any one of SEQ ID NOs: 210-211, 213-229, 231-232, 235-248, 250-287, 710 and 724-725, or an amino acid sequence that is at least 80%, at least 85%, at least 90%, at least 91%, at least 92%, at least 93%, at least 94%, at least 95%, at least 96%, at least 97%, at least 98%, or at least 99% identical, to one of the amino acid sequences of any one of SEQ ID NOs: 210-211, 213-229, 231-232, 235-248, 250-287, 710 and 724-725.

“Homology” or “identity” or “similarity” as used herein in the context of nucleic acids and polypeptides refers to the relationship between two polypeptides or two nucleic acid molecules based on an alignment of the amino acid sequences or nucleic acid sequences, respectively. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. In comparing two sequences, the absence of residues (amino acids or nucleic acids) or presence of extra residues also decreases the identity and homology/similarity.

As used herein, “identity” means the percentage of identical nucleotide or amino acid residues at corresponding positions in two or more sequences when the sequences are aligned to maximize sequence matching, i.e., taking into account gaps and insertions. Sequences are generally aligned for maximum correspondence over a designated region, e.g., a region at least 20, 25, 30, 35, 40, 45, 50, 55, 60, 65 or more amino acids or nucleotides in length, and can be up to the full length of the reference polypeptide or polynucleotide sequence. For sequence comparison, typically one sequence acts as a reference sequence, to which test sequences are compared. When using a sequence comparison algorithm, test and reference sequences are input into a computer program, subsequence coordinates are designated, if necessary, and sequence algorithm program parameters are designated. Otherwise, standard parameters can be used. The sequence comparison algorithm then calculates the percent sequence identity for the test sequence(s) relative to the reference sequence, based on the designated program parameters.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least 20 contiguous positions, usually 30 to 75, 40 to 50, or the full length of a sequence, in which a sequence may be compared to a reference sequence of the same number of contiguous positions after the two sequences are optimally aligned.

Optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (e.g., GAP, BESTFIT, BLAST, FASTA, and TFASTA in the Wisconsin Genetics Software Package, Genetics Computer Group (GCG), 575 Science Dr., Madison, WI), or by inspection.

One example of algorithms that are suitable for determining percent sequence identity are the Basic Local Alignment Search Tool (BLAST), BLAST 2.0 and PSI-BLAST algorithms, which are described in Altschul, et al., J. Mol. Biol. (1990) 215: 403-410, Altschul, et al., Nucleic Acids Res. (1977) 25: 3389-3402, and Altschul, et al., Nucleic Acids Res. (1997) 25(17):3389-402, respectively. Software for performing BLAST analyses is publicly available through the National Center for Biotechnology Information (blast.ncbi.nlm.nih.gov/Blast.cgi).

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTP program (for polypeptide sequences) or the BLASTN program (for polynucleotide sequences) uses as defaults a word length (W) of 11, and expectation (E) of 10, and the BLOSUM62 scoring matrix (see Henikoff and Henikoff (1989) Proc. Natl. Acad. Sci. USA 89: 10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLASTP algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Residue positions which are not identical can differ by conservative amino acid substitutions. Conservative amino acid substitutions refer to the interchangeability of residues having similar side chains. For example, a group of amino acids having aliphatic side chains is glycine (Gly, G), alanine (Ala, A), valine (Val, V), leucine (Leu, L), and isoleucine (Ile, I); a group of amino acids having aliphatic-hydroxyl side chains is serine (Ser, S) and threonine (Thr, T); a group of amino acids having amide-containing side chains is asparagine (Asn, N) and glutamine (Gln, Q); a group of amino acids having aromatic side chains is phenylalanine (Phe, F), tyrosine (Tyr, Y), and tryptophan (Trp, W); a group of amino acids having basic side chains is lysine (Lys, K), arginine (Arg, R), and histidine (His, H); and a group of amino acids having sulfur-containing side chains is cysteine (Cys, C) and methionine (Met, M). Further, glutamic acid (Glu, E) and aspartic acid (Asp, D) are conservative amino acid substitutions.

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 1, 2 and 3 (according to Kabat);
2) SEQ ID NOs: 56, 57 and 58 (according to IMGT);
3) SEQ ID NOs: 108, 109 and 110 (according to Chothia); or
4) SEQ ID NOs: 153, 154 and 155 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 210-211, or an amino acid sequence at least 90%, 91%, 92%, 93%, 94%, 95%, 96%, 97%, 98%, 99% identical to, respectively, the full length of the amino acid sequence set forth in any one of SEQ ID NOs: 210-211 (VHH A01).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 5 and 6 (according to Kabat);
2) SEQ ID NOs: 59, 60 and 61 (according to IMGT);
3) SEQ ID NOs: 111, 112 and 113 (according to Chothia); or
4) SEQ ID NOs: 156, 157 and 158 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 212-215, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 212-215 (VHH A06).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 7, 8 and 9 (according to Kabat);
2) SEQ ID NOs: 62, 63 and 64 (according to IMGT);
3) SEQ ID NOs: 114, 115 and 116 (according to Chothia); or
4) SEQ ID NOs: 159, 160 and 161 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 216-217, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 216-217 (VHH A09).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 10, 11 and 12 (according to Kabat);
2) SEQ ID NOs: 65, 66 and 67 (according to IMGT);
3) SEQ ID NOs: 117, 118 and 119 (according to Chothia); or
4) SEQ ID NOs: 162, 163 and 164 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 218-219, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 218-219 (VHH B07).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 13 and 14 (according to Kabat);
2) SEQ ID NOs: 59, 68 and 69 (according to IMGT);
3) SEQ ID NOs: 111, 112 and 120 (according to Chothia); or
4) SEQ ID NOs: 156, 165 and 166 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 220-223, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 220-223 (VHH B08).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 15 and 16 (according to Kabat);
2) SEQ ID NOs: 59, 70 and 71 (according to IMGT);
3) SEQ ID NOs: 111, 121 and 122 (according to Chothia); or
4) SEQ ID NOs: 156, 167 and 168 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 224-226 and 710, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 224-226 and 710 (VHH C09).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 17 and 18 (according to Kabat);
2) SEQ ID NOs: 59, 72 and 73 (according to IMGT);
3) SEQ ID NOs: 111, 121 and 123 (according to Chothia); or
4) SEQ ID NOs: 156, 169 and 170 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 227-229 and 710, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 227-229 and 710 (VHH D08).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 19, 20 and 21 (according to Kabat);
2) SEQ ID NOs: 74, 75 and 76 (according to IMGT);
3) SEQ ID NOs: 124, 125 and 126 (according to Chothia); or
4) SEQ ID NOs: 171, 172 and 173 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 230-232, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 230-232 (VHH D14).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 22, 23 and 24; or 22, 25 and 26; or 22, 27 and 24 (according to Kabat); 2) SEQ ID NOs: 77, 78 and 79; or 77, 80 and 81; or 77, 82 and 79 (according to IMGT); 3) SEQ ID NOs: 127, 128 and 129; or 127, 130 and 131 (according to Chothia); or 4) SEQ ID NOs: 174, 175 and 176; or 174, 177 and 178; or 174, 179 and 176 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 233-240 and 724-725, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 233-240 and 724-725. In some embodiments, the VHH binds to an epitope on IL 10Rα comprising contact amino acid residues W33, E35, E37, H40, 142, H44, T46, L79, S80, T124, A170, R172, F178, F180, H182, Q204, K215, M217, W218, K220 and E221, wherein the amino acid residue positions are with respect to SEQ ID NO: 711. (VHH E07).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 28 and 29 (according to Kabat);
2) SEQ ID NOs: 59, 83 and 84 (according to IMGT);
3) SEQ ID NOs: 111, 132 and 133 (according to Chothia); or
4) SEQ ID NOs: 156, 180 and 181 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 241-243 and 710, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 241-243 and 710 (VHH E15).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 30 and 31 (according to Kabat);
2) SEQ ID NOs: 59, 85 and 86 (according to IMGT);
3) SEQ ID NOs: 111, 134 and 135 (according to Chothia); or
4) SEQ ID NOs: 156, 182 and 183 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 244-246, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 244-246 (VHH F09).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 22, 23 and 32 (according to Kabat);
2) SEQ ID NOs: 77, 78 and 87 (according to IMGT);
3) SEQ ID NOs: 127, 128 and 136 (according to Chothia); or
4) SEQ ID NOs: 174, 175 and 184 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 247-248, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 247-248 (VHH F10).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 33, 34 and 35 (according to Kabat);
2) SEQ ID NOs: 88, 89 and 90 (according to IMGT);
3) SEQ ID NOs: 137, 138 and 139 (according to Chothia); or
4) SEQ ID NOs: 185, 186 and 187 or 709, 186 and 187 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 249-252 and 752-755, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 249-252 and 752-755. In some embodiments, the VHH binds to an epitope on IL 10Rα comprising contact amino acid residues I169, I171, K173, F178, F180, T181, H182, K183, K186, F190, L192, L193, S195, E197 and F201, wherein the amino acid residue positions are with respect to SEQ ID NO: 711. (VHH F11).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 36 and 29 (according to Kabat);
2) SEQ ID NOs: 59, 91 and 84 (according to IMGT);
3) SEQ ID NOs: 111, 132 and 133 (according to Chothia); or
4) SEQ ID NOs: 156, 188 and 181 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 253-255 and 710, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 253-255 and 710 (VHH F21).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 19, 37 and 38 (according to Kabat);
2) SEQ ID NOs: 92, 75 and 93 (according to IMGT);
3) SEQ ID NOs: 140, 125 and 141 (according to Chothia); or
4) SEQ ID NOs: 189, 190 and 191 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 230 and 256-259, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 230 and 256-259 (VHH G22).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 19, 37 and 38 (according to Kabat);
2) SEQ ID NOs: 92, 75 and 93 (according to IMGT);
3) SEQ ID NOs: 140, 125 and 141 (according to Chothia); or
4) SEQ ID NOs: 189, 190 and 191 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 260-263, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 260-263 (VHH G23).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 42, 43 and 44 (according to Kabat);
2) SEQ ID NOs: 96, 89 and 97 (according to IMGT);
3) SEQ ID NOs: 124, 138 and 144 (according to Chothia); or
4) SEQ ID NOs: 195, 196 and 197 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 264-266, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 264-266 (VHH H22).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 45, 46 and 47 (according to Kabat);
2) SEQ ID NOs: 98, 99 and 100 (according to IMGT);
3) SEQ ID NOs: 145, 146 and 147 (according to Chothia); or
4) SEQ ID NOs: 198, 199 and 200 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any of SEQ ID NOs: 267-269, or an amino acid sequence at least 90%, 91%, 92%, 93%, 94%, 95%, 96%, 97%, 98%, 99% identical to, respectively, the full length of any one of the amino acid sequences set forth in any of SEQ ID NOs: 267-269 (VHH I04).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 48 and 6 (according to Kabat);
2) SEQ ID NOs: 59, 101 and 61 (according to IMGT);
3) SEQ ID NOs: 111, 112 and 113 (according to Chothia); or
4) SEQ ID NOs: 156, 201 and 158 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 270-273, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 270-273 (VHH J13).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 4, 49 and 50 (according to Kabat);
2) SEQ ID NOs: 59, 102 and 103 (according to IMGT);
3) SEQ ID NOs: 111, 148 and 149 (according to Chothia); or
4) SEQ ID NOs: 156, 202 and 203 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 274-277, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 274-277 (VHH J24).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 19, 51 and 52 (according to Kabat);
2) SEQ ID NOs: 74, 75 and 104 (according to IMGT);
3) SEQ ID NOs: 124, 125 and 150 (according to Chothia); or
4) SEQ ID NOs: 204, 205 and 206 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 230 and 278-280, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 230 and 278-280 (VHH L05).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 19, 37 and 53 (according to Kabat);
2) SEQ ID NOs: 74, 75 and 105 (according to IMGT);
3) SEQ ID NOs: 124, 125 and 151 (according to Chothia); or
4) SEQ ID NOs: 204, 190 and 207 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 230 and 281-284, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 230 and 281-284 (VHH L11).

In some embodiments, provided is a single domain antibody (sdAb) or antigen-binding fragment thereof (VHH) that specifically binds to IL-10Rα comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 54, 55 and 24 (according to Kabat);
2) SEQ ID NOs: 106, 107 and 79 (according to IMGT);
3) SEQ ID NOs: 152, 121 and 129 (according to Chothia); or
4) SEQ ID NOs: 208, 209 and 176 (according to Honegger).
   In some embodiments, provided is a single domain antibody (sdAb) or antigen-binding fragment thereof (VHH) that specifically binds to IL-10Rα, wherein the VH and VL comprise, respectively, the amino acid sequences set forth in any one of SEQ ID NOs: 285-287, or an amino acid sequence at least 90%, 91%, 92%, 93%, 94%, 95%, 96%, 97%, 98%, 99% identical to, respectively, the full length of any one of the amino acid sequences set forth in any one of SEQ ID NOs: 285-287 (VHH N16).

Generally, the herein described VHH that bind to IL-10Rα bind to both human IL-10Rα and cynomolgus IL-10Rα, within a 10-fold affinity difference, e.g., within a 5-fold or within a 3-fold affinity difference (e.g., as determined by SPR and expressed as an equilibrium dissociation constant (KD)). Amino acid sequences of complementarity determining regions (CDRs) of illustrative VHH that bind to IL-10Rα are provided in Tables A1-A4. Amino acid sequences of variable regions of illustrative VHH that bind to IL-10Rα are provided in Table B.

TABLE A1
Illustrative IL-10Rα Single Immunoglobulin Variable
domain (VHH) Binder CDR amino acid sequences
(according to Kabat)

| VHH name | VHH - CDR1 | VHH - CDR2 | VHH - CDR3 |
|---|---|---|---|
| A01 | NYWVD | AINKDGGSAYYADSVKG | DYGSGMHD |
| | SEQ ID NO: 1 | SEQ ID NO: 2 | SEQ ID NO: 3 |
| A06 | SYGMG | AINWIPGTTYYADSVKG | RRRIDNYYRASEYDY |
| | SEQ ID NO: 4 | SEQ ID NO: 5 | SEQ ID NO: 6 |
| A09 | SYAID | CISSSGISTTYADSVKG | AALCMSGTYYAGPGY |
| | SEQ ID NO: 7 | SEQ ID NO: 8 | SEQ ID NO: 9 |
| B07 | YYAIG | CISSSGGSTYYADSVKG | DAACMVVAGTLILPDDY |
| | SEQ ID NO: 10 | SEQ ID NO: 11 | SEQ ID NO: 12 |
| B08 | SYGMG | TINWIPGSTYYADSVKG | RRRIESYYRASEYDY |
| | SEQ ID NO: 4 | SEQ ID NO: 13 | SEQ ID NO: 14 |
| C09 | SYGMG | AISWIGGGTYYADSVKG | KYYPDSYFAAASYAY |
| | SEQ ID NO: 4 | SEQ ID NO: 15 | SEQ ID NO: 16 |
| D08 | SYGMG | AISWIGGSTYYADSVKG | KYYTDSYFAATSYAY |
| | SEQ ID NO: 4 | SEQ ID NO: 17 | SEQ ID NO: 18 |
| D14 | GYGMG | SINWIYGATYYTDSVKG | KINGGSYFNTGSFGF |
| | SEQ ID NO: 19 | SEQ ID NO: 20 | SEQ ID NO: 21 |
| E07 | AYGMG | AIRWIGDSTYYADSVKG | KTSVTSYLSEKSYNY |
| | SEQ ID NO: 22 | SEQ ID NO: 23 | SEQ ID NO: 24 |
| huE07 | AYGMG | AIRWYGEGTYYAESVKG | KTRPDSYLSEKSYGY |
| | SEQ ID NO: 22 | SEQ ID NO: 25 | SEQ ID NO: 26 |
| huE07 | AYGMG | AIRWIGDATYYAESVKG | KTSVTSYLSEKSYNY |
| | SEQ ID NO: 22 | SEQ ID NO: 27 | SEQ ID NO: 24 |
| E15 | SYGMG | AISWIGNSAYYADSVKG | KYYTDSYYAAASYAY |
| | SEQ ID NO: 4 | SEQ ID NO: 28 | SEQ ID NO: 29 |
| F09 | SYGMG | AISWIGSATDYADSVRG | RRSPMSRTSVLQYDY |
| | SEQ ID NO: 4 | SEQ ID NO: 30 | SEQ ID NO: 31 |
| F10 | AYGMG | AIRWIGDSTYYADSVKG | KTSVTSYLSEKAYNY |
| | SEQ ID NO: 22 | SEQ ID NO: 23 | SEQ ID NO: 32 |
| F11 | SHTVG | AITWNGDTTYFGDFVEG | ATGQVWSLQYLRESSYAY |
| | SEQ ID NO: 33 | SEQ ID NO: 34 | SEQ ID NO: 35 |
| F21 | SYGMG | AISWIGNSTYYADSVKG | KYYTDSYYAAASYAY |
| | SEQ ID NO: 4 | SEQ ID NO: 36 | SEQ ID NO: 29 |
| G22 | GYGMG | AINWIYGATYYADSVKG | KINAGSYFNIGSFGS |
| | SEQ ID NO: 19 | SEQ ID NO: 37 | SEQ ID NO: 38 |
| G23 | DDTVG | AITWNGDTTYYGDSVKG | AAGQDWSLGYLRESSYAY |
| | SEQ ID NO: 39 | SEQ ID NO: 40 | SEQ ID NO: 41 |
| H22 | GYTVG | AITWNGDTTYFGDSVKG | ATGQEWSLQYLRESSYAY |
| | SEQ ID NO: 42 | SEQ ID NO: 43 | SEQ ID NO: 44 |
| I04 | NYAMA | AINWFGRATEYADSVKD | RTKVTTGRRNQAEYDY |
| | SEQ ID NO: 45 | SEQ ID NO: 46 | SEQ ID NO: 47 |
| J13 | SYGMG | TINWIPGNTYYADSVKG | RRRIDNYYRASEYDY |
| | SEQ ID NO: 4 | SEQ ID NO: 48 | SEQ ID NO: 6 |
| J24 | SYGMG | TIIGWIPGNTYYADSVKG | RRRIDSYYRASEYDY |
| | SEQ ID NO: 4 | SEQ ID NO: 49 | SEQ ID NO: 50 |
| L05 | GYGMG | SINWIYGATYYSDSVKG | KINGESYFNTGSFGS |
| | SEQ ID NO: 19 | SEQ ID NO: 51 | SEQ ID NO: 52 |
| L11 | GYGMG | AINWIYGATYYADSVKG | KINGGSYFNIGSFGS |
| | SEQ ID NO: 19 | SEQ ID NO: 37 | SEQ ID NO: 53 |
| N16 | YYGMG | AIRWIGGDTYYADSVKG | KTSVTSYLSEKSYNY |
| | SEQ ID NO: 54 | SEQ ID NO: 55 | SEQ ID NO: 24 |

TABLE A2
Illustrative IL-10Rα Single Immunoglobulin
Variable domain (VHH) Binder CDR amino acid sequences
(according to IMGT)

| VHH name | VHH - CDR1 | VHH - CDR2 | VHH - CDR3 |
|---|---|---|---|
| A01 | GFTFSNYW | INKDGGSA | AKDYGSGMHD |
| | SEQ ID NO: 56 | SEQ ID NO: 57 | SEQ ID NO: 58 |
| A06 | GRTFSSYG | INWIPGTT | AARRRIDNYYRASEYDY |
| | SEQ ID NO: 59 | SEQ ID NO: 60 | SEQ ID NO: 61 |
| A09 | GFTLDSYA | ISSSGIST | AAAALCMSGTYYAGPGY |
| | SEQ ID NO: 62 | SEQ ID NO: 63 | SEQ ID NO: 64 |
| B07 | DFTLDYYA | ISSSGGST | AADAACMVVAGTLILPDDY |
| | SEQ ID NO: 65 | SEQ ID NO: 66 | SEQ ID NO: 67 |
| B08 | GRTFSSYG | INWIPGST | AARRRIESYYRASEYDY |
| | SEQ ID NO: 59 | SEQ ID NO: 68 | SEQ ID NO: 69 |
| C09 | GRTFSSYG | ISWIGGGT | AAKYYPDSYFAAASYAY |
| | SEQ ID NO: 59 | SEQ ID NO: 70 | SEQ ID NO: 71 |
| D08 | GRTFSSYG | ISWIGGST | AAKYYTDSYFAATSYAY |
| | SEQ ID NO: 59 | SEQ ID NO: 72 | SEQ ID NO: 73 |
| D14 | GRTFSGYG | INWIYGAT | AAKINGGSYFNTGSFGF |
| | SEQ ID NO: 74 | SEQ ID NO: 75 | SEQ ID NO: 76 |
| E07 | GRTFSAYG | IRWIGDST | AAKTSVTSYLSEKSYNY |
| | SEQ ID NO: 77 | SEQ ID NO: 78 | SEQ ID NO: 79 |
| huE07 | GRTFSAYG | IRWYGEGT | AAKTRPDSYLSEKSYGY |
| | SEQ ID NO: 77 | SEQ ID NO: 80 | SEQ ID NO: 81 |
| huE07 | GRTFSAYG | IRWIGDAT | AAKTSVTSYLSEKSYNY |
| | SEQ ID NO: 77 | SEQ ID NO: 82 | SEQ ID NO: 79 |
| E15 | GRTFSSYG | ISWIGNSA | AAKYYTDSYYAAASYAY |
| | SEQ ID NO: 59 | SEQ ID NO: 83 | SEQ ID NO: 84 |
| F09 | GRTFSSYG | ISWIGSAT | AARRSPMSRTSVLQYDY |
| | SEQ ID NO: 59 | SEQ ID NO: 85 | SEQ ID NO: 86 |
| F10 | GRTFSAYG | IRWIGDST | AAKTSVTSYLSEKAYNY |
| | SEQ ID NO: 77 | SEQ ID NO: 78 | SEQ ID NO: 87 |
| F11 | GRTFSSHT | ITWNGDTT | AAATGQVWSLQYLRESSYAY |
| | SEQ ID NO: 88 | SEQ ID NO: 89 | SEQ ID NO: 90 |
| F21 | GRTFSSYG | ISWIGNST | AAKYYTDSYYAAASYAY |
| | SEQ ID NO: 59 | SEQ ID NO: 91 | SEQ ID NO: 84 |
| G22 | GRTFDGYG | INWIYGAT | AAKINAGSYFNIGSFGS |
| | SEQ ID NO: 92 | SEQ ID NO: 75 | SEQ ID NO: 93 |
| G23 | GRTFSDDT | ITWNGDTT | AAAAGQDWSLGYLRESSYAY |
| | SEQ ID NO: 94 | SEQ ID NO: 89 | SEQ ID NO: 95 |
| H22 | GRTFSGYT | ITWNGDTT | AAATGQEWSLQYLRESSYAY |
| | SEQ ID NO: 96 | SEQ ID NO: 89 | SEQ ID NO: 97 |
| I04 | GRTFSNYA | INWF GRAT | AARTKVTTGRRNQAEYDY |
| | SEQ ID NO: 98 | SEQ ID NO: 99 | SEQ ID NO: 100 |
| J13 | GRTFSSYG | INWIPGNT | AARRRIDNYYRASEYDY |
| | SEQ ID NO: 59 | SEQ ID NO: 101 | SEQ ID NO: 61 |
| J24 | GRTFSSYG | IIGWIPGNT | AARRRIDSYYRASEYDY |
| | SEQ ID NO: 59 | SEQ ID NO: 102 | SEQ ID NO: 103 |
| L05 | GRTFSGYG | INWIYGAT | AAKINGESYENTGSFGS |
| | SEQ ID NO: 74 | SEQ ID NO: 75 | SEQ ID NO: 104 |
| L11 | GRTFSGYG | INWIYGAT | AAKINGGSYFNIGSFGS |
| | SEQ ID NO: 74 | SEQ ID NO: 75 | SEQ ID NO: 105 |
| N16 | GGTFSYYG | IRWIGGDT | AAKTSVTSYLSEKSYNY |
| | SEQ ID NO: 106 | SEQ ID NO: 107 | SEQ ID NO: 79 |

TABLE A3
Illustrative IL-10Rα Single Immunoglobulin
Variable domain (VHH) Binder CDR amino acid sequences
(according to Chothia)

| VHH name | VHH - CDR1 | VHH - CDR2 | VHH - CDR3 |
|---|---|---|---|
| A01 | GFTFSNY | KDGG | YGSGMH |
| | SEQ ID NO: 108 | SEQ ID NO: 109 | SEQ ID NO: 110 |
| A06 | GRIFSSY | WIPG | RRIDNYYRASEYD |
| | SEQ ID NO: 111 | SEQ ID NO: 112 | SEQ ID NO: 113 |
| A09 | GFTLDSY | SSGI | ALCMSGTYYAGPG |
| | SEQ ID NO: 114 | SEQ ID NO: 115 | SEQ ID NO: 116 |
| B07 | DFTLDYY | SSGG | AACMVVAGTLILPDD |
| | SEQ ID NO: 117 | SEQ ID NO: 118 | SEQ ID NO: 119 |
| B08 | GRTFSSY | WIPG | RRIESYYRASEYD |
| | SEQ ID NO: 111 | SEQ ID NO: 112 | SEQ ID NO: 120 |
| C09 | GRTFSSY | WIGG | YYPDSYFAAASYA |
| | SEQ ID NO: 111 | SEQ ID NO: 121 | SEQ ID NO: 122 |
| D08 | GRIFSSY | WIGG | YYTD SYFAATSYA |
| | SEQ ID NO: 111 | SEQ ID NO: 121 | SEQ ID NO: 123 |
| D14 | GRTFSGY | WIYG | INGGSYFNTGSFG |
| | SEQ ID NO: 124 | SEQ ID NO: 125 | SEQ ID NO: 126 |
| E07 | GRTFSAY | WIGD | TSVTSYLSEKSYN |
| | SEQ ID NO: 127 | SEQ ID NO: 128 | SEQ ID NO: 129 |
| huE07 | GRTFSAY | WYGE | TRPDSYLSEKSYG |
| | SEQ ID NO: 127 | SEQ ID NO: 130 | SEQ ID NO: 131 |
| E15 | GRTFSSY | WIGN | YYTDSYYAAASYA |
| | SEQ ID NO: 111 | SEQ ID NO: 132 | SEQ ID NO: 133 |
| F09 | GRTESSY | WIGS | RSPMSRTSVLQYD |
| | SEQ ID NO: 111 | SEQ ID NO: 134 | SEQ ID NO: 135 |
| F10 | GRTFSAY | WIGD | TSVTSYLSEKAYN |
| | SEQ ID NO: 127 | SEQ ID NO: 128 | SEQ ID NO: 136 |
| F11 | GRTFSSH | WNGD | TGQVWSLQYLRESSYA |
| | SEQ ID NO: 137 | SEQ ID NO: 138 | SEQ ID NO: 139 |
| F21 | GRTFSSY | WIGN | YYTDSYYAAASYA |
| | SEQ ID NO: 111 | SEQ ID NO: 132 | SEQ ID NO: 133 |
| G22 | GRTFDGY | WIYG | INAGSYFNIGSFG |
| | SEQ ID NO: 140 | SEQ ID NO: 125 | SEQ ID NO: 141 |
| G23 | GRTFSDD | WNGD | AGQDWSLGYLRESSYA |
| | SEQ ID NO: 142 | SEQ ID NO: 138 | SEQ ID NO: 143 |
| H22 | GRTFSGY | WNGD | TGQEWSLQYLRESSYA |
| | SEQ ID NO: 124 | SEQ ID NO: 138 | SEQ ID NO: 144 |
| I04 | GRTFSNY | WFGR | TKVTTGRRNQAEYD |
| | SEQ ID NO: 145 | SEQ ID NO: 146 | SEQ ID NO: 147 |
| J13 | GRTFSSY | WIPG | RRIDNYYRASEYD |
| | SEQ ID NO: 111 | SEQ ID NO: 112 | SEQ ID NO: 113 |
| J24 | GRTFSSY | GWIPG | RRIDSYYRASEYD |
| | SEQ ID NO: 111 | SEQ ID NO: 148 | SEQ ID NO: 149 |
| L05 | GRTFSGY | WIYG | INGESYFNTGSFG |
| | SEQ ID NO: 124 | SEQ ID NO: 125 | SEQ ID NO: 150 |
| L11 | GRTFSGY | WIYG | INGGSYFNIGSFG |
| | SEQ ID NO: 124 | SEQ ID NO: 125 | SEQ ID NO: 151 |
| N16 | GGTFSYY | WIGG | TSVTSYLSEKSYN |
| | SEQ ID NO: 152 | SEQ ID NO: 121 | SEQ ID NO: 129 |

TABLE A4
Illustrative IL-10Rα Single Immunoglobulin
Variable domain (VHH) Binder CDR amino acid sequences
(according to Honegger)

| VHH | | | |
|---|---|---|---|
| name | VHH - CDR1 | VHH - CDR2 | VHH - CDR3 |
| A01 | ASGFTF SNYW | INKDGGSAYYADSVKGR | DYGSGMH |
| | SEQ ID NO: 153 | SEQ ID NO: 154 | SEQ ID NO: 155 |
| A06 | ASGRTFSSYG | INWIPGTTYYADSVKGR | RRRIDNYYRASEYD |
| | SEQ ID NO: 156 | SEQ ID NO: 157 | SEQ ID NO: 158 |
| A09 | ASGFTLDSYA | ISSSGISTTYADSVKGR | AALCMSGTYYAGPG |
| | SEQ ID NO: 159 | SEQ ID NO: 160 | SEQ ID NO: 161 |
| B07 | ASDFTLDYYA | ISSSGGSTYYADSVKGR | DAACMVVAGTLILPDD |
| | SEQ ID NO: 162 | SEQ ID NO: 163 | SEQ ID NO: 164 |
| B08 | ASGRTFSSYG | INWIPGSTYYADSVKGR | RRRIESYYRASEYD |
| | SEQ ID NO: 156 | SEQ ID NO: 165 | SEQ ID NO: 166 |
| C09 | ASGRTFSSYG | ISWIGGGTYYADSVKGR | KYYPDSYFAAASYA |
| | SEQ ID NO: 156 | SEQ ID NO: 167 | SEQ ID NO: 168 |
| D08 | ASGRTFSSYG | ISWIGGSTYYADSVKGR | KYYTDSYFAATSYA |
| | SEQ ID NO: 156 | SEQ ID NO: 169 | SEQ ID NO: 170 |
| D14 | ASGRTFSGYG | INWIYGATYYTDSVKGR | KINGGSYFNTGSFG |
| | SEQ ID NO: 171 | SEQ ID NO: 172 | SEQ ID NO: 173 |
| E07 | ASGRTFSAYG | IRWIGDSTYYADSVKGR | KTSVTSYLSEKSYN |
| | SEQ ID NO: 174 | SEQ ID NO: 175 | SEQ ID NO: 176 |
| huE07 | ASGRTFSAYG | IRWYGEGTYYAESVKGR | KTRPDSYLSEKSYG |
| | SEQ ID NO: 174 | SEQ ID NO: 177 | SEQ ID NO: 178 |
| huE07 | ASGRTFSAYG | IRWIGDATYYAESVKGR | KTSVTSYLSEKSYN |
| | SEQ ID NO: 174 | SEQ ID NO: 179 | SEQ ID NO: 176 |
| E15 | ASGRTFSSYG | ISWIGNSAYYADSVKGR | KYYTDSYYAAASYA |
| | SEQ ID NO: 156 | SEQ ID NO: 180 | SEQ ID NO: 181 |
| F09 | ASGRTFSSYG | ISWIGSATDYADSVRGR | RRSPMSRTSVLQYD |
| | SEQ ID NO: 156 | SEQ ID NO: 182 | SEQ ID NO: 183 |
| F10 | ASGRTFSAYG | IRWIGDSTYYADSVKGR | KTSVTSYLSEKAYN |
| | SEQ ID NO: 174 | SEQ ID NO: 175 | SEQ ID NO: 184 |
| F11 | NSGRTFSSHT | ITWNGDTTYFGDFVEGR | ATGQVWSLQYLRESSYA |
| | SEQ ID NO: 185 | SEQ ID NO: 186 | SEQ ID NO: 187 |
| huF11 | ASGRTFSSHT | ITWNGDTTYFGDFVEGR | ATGQVWSLQYLRESSYA |
| | SEQ ID NO: 709 | SEQ ID NO: 186 | SEQ ID NO: 187 |
| F21 | ASGRTFSSYG | ISWIGNSTYYADSVKGR | KYYTDSYYAAASYA |
| | SEQ ID NO: 156 | SEQ ID NO: 188 | SEQ ID NO: 181 |
| G22 | ASGRTFDGYG | INWIYGATYYADSVKGR | KINAGSYFNIGSFG |
| | SEQ ID NO: 189 | SEQ ID NO: 190 | SEQ ID NO: 191 |
| G23 | ASGRTFSDDT | ITWNGDTTYYGDSVKGR | AAGQDWSLGYLRESSYA |
| | SEQ ID NO: 192 | SEQ ID NO: 193 | SEQ ID NO: 194 |
| H22 | ASGRTFSGYT | ITWNGDTTYFGDSVKGR | ATGQEWSLQYLRESSYA |
| | SEQ ID NO: 195 | SEQ ID NO: 196 | SEQ ID NO: 197 |
| I04 | ASGRTFSNYA | INWFGRATEYADSVKDR | RTKVTTGRRNQAEYD |
| | SEQ ID NO: 198 | SEQ ID NO: 199 | SEQ ID NO: 200 |
| J13 | ASGRTFSSYG | INWIPGNTYYADSVKGR | RRRIDNYYRASEYD |
| | SEQ ID NO: 156 | SEQ ID NO: 201 | SEQ ID NO: 158 |
| J24 | ASGRTFSSYG | IIGWIPGNTYYADSVKGR | RRRIDSYYRASEYD |
| | SEQ ID NO: 156 | SEQ ID NO: 202 | SEQ ID NO: 203 |
| L05 | NSGRTFSGYG | INWIYGATYYSDSVKGR | KINGESYFNTGSEG |
| | SEQ ID NO: 204 | SEQ ID NO: 205 | SEQ ID NO: 206 |
| L11 | NSGRTFSGYG | INWIYGATYYADSVKGR | KINGGSYFNIGSFG |
| | SEQ ID NO: 204 | SEQ ID NO: 190 | SEQ ID NO: 207 |
| N16 | ASGGTFSYYG | IRWIGGDTYYADSVKGR | KTSVTSYLSEKSYN |
| | SEQ ID NO: 208 | SEQ ID NO: 209 | SEQ ID NO: 176 |

TABLE B
Illustrative IL-10Rα Single Immunoglobulin Variable
Domain (VHH) amino acid sequences

| VHH | |
|---|---|
| name | VHH amino acid sequence |
| A01 | SEQ ID NO: 210 |
| | DVQLVESGGGVVQPGGSLRLSCAASGFTFSNYWVDWLRQAPGKGLEWVSAINKDG |
| | GSAYYADSVKGRFTISRDNAKNTLYLQMTSLKPEDTAVYYCAKDYGSGMHDWGKG |
| | TQVTVSS |
| huA01 | SEQ ID NO: 211 |
| | EVQLVESGGGLVQPGGSLRLSCAASGFTFSNYWVDWLRQAPGKGLEWVSAINKDG |
| | GSAYYADSVKGRFTISRDNSKNTLYLQMNSLRAEDTAVYYCAKDYGSGMHDWGQG |
| | TMVTVSS |
| A06 | SEQ ID NO: 212 |
| con | X₁X₂LQLVESGGGX₃VQX₄GGSLRLSCAASGRTFSSYGMGWFRQAPGKERX₅X₆VA |
| | X₇IX₈NWIPGX₉TYYADSVKGRFTIX₁₀GDKX₁₁KNTLYLQMDX₁₂LX₁₃X₁₄EDTAVYY |
| | CAARRRIX₁₅X₁₆YYRASEYDYWGQGTX₁₇VTVSS |
| | X₁ is S, GG, AS, ASS or absent (i.e., no amino acid |
| | residues) |
| | X₂ is E or Q |
| | X₃ is L, S or V |
| | X₄ is A or P |
| | X₅ is E or G |
| | X₆ is F or L |
| | X₇ is A or T |
| | X₈ is I or absent |
| | X₉ is N, S or T |
| | X₁₀ is S or T |
| | X₁₁ is D or S |
| | X₁₂ is N or S |
| | X₁₃ is K or R |
| | X₁₄ is A or P |
| | X₁₅ is D or E |
| | X₁₆ is N or S |
| | X₁₇ is L or Q |
| A06 | SEQ ID NO: 213 |
| | X₁ is S, GG, AS, ASS or absent (i.e., no amino acid |
| | residues ) |
| | SEQ ID NO: 214 |
| | X₁ is AS |
| | X₁QLQLVESGGGLVQAGGSLRLSCAASGRTFSSYGMGWFRQAPGKERGLVAAINWI |
| | PGTTYYADSVKGRFTI SGDKDKNTLYLQMDNLKPEDTAVYYCAARRRIDNYYRAS |
| | EYDYWGQGTQVTVSS |
| huA06 | SEQ ID NO: 215 |
| | ELQLVESGGGLVQPGGSLRLSCAASGRTFSSYGMGWFRQAPGKERELVAAINWIP |
| | GTTYYADSVKGRFTISGDKSKNTLYLQMNSLRAEDTAVYYCAARRRIDNYYRASE |
| | YDYWGQGTLVTVSS |
| A09 | SEQ ID NO: 216 |
| | DLQLVESGGGLVQPGGSLRLSCAASGFTLDSYAIDWFRQAPGKEREGVSCISSSG |
| | ISTTYADSVKGRFTISRDNAKNTVYLQMNSLKPEDTAVYYCAAAALCMSGTYYAG |
| | PGYWGQGTQVTVSS |
| huA09 | SEQ ID NO: 217 |
| | ELQLVESGGGLVQPGGSLRLSCAASGFTLDSYAIDWFRQAPGKEREGVSCISSSG |
| | ISTTYADSVKGRFTISRDNSKNTVYLQMNSLRAEDTAVYYCAAAALCMSGTYYAG |
| | PGYWGQGTTVTVSS |
| B07 | SEQ ID NO: 218 |
| | DVQLVESGGGLVQPGGSLRLSCAASDFTLDYYAIGWFRQAPGKEREGVSCISSSG |
| | GSTYYADSVKGRFTISRDNAKNTVSLQMNSLKPEDTAVYYCAADAACMVVAGTLI |
| | LPDDYWGQGTQVTVSS |
| huB07 | SEQ ID NO: 219 |
| | EVQLVESGGGLVQPGGSLRLSCAASGFTLDYYAIGWFRQAPGKEREGVSCISSSG |
| | GSTYYADSVKGRFTISRDNSKNTVYLQMNSLRAEDTAVYYCAADAACMVVAGTLI |
| | LPDDYWGQGTLVTVSS |
| B08 | SEQ ID NO: 220 |
| | X₁ is S, GG, AS, ASS or absent (i.e., no amino acid |
| | residues ) |
| | SEQ ID NO: 221 |
| | X₁ is AS |
| | X₁QVQLVESGGGSVQAGGSLRLSCAASGRTFSSYGMGWFRQAPGKERGEVATINWI |
| | PGSTYYADSVKGRFTISGDKDNNTVYLQMDNLKPEDTAVYYCAARRRIESYYRAS |
| | EYDYWGQGTQVTVSS |
| huB08 | SEQ ID NO: 222 |
| | X₁ is S, GG, AS, ASS or absent (i.e., no amino acid |
| | residues ) |
| | SEQ ID NO: 223 |
| | X₁ is AS |
| | X₁QVQLVESGGGVVQPGGSLRLSCAASGRTFSSYGMGWFRQAPGKEREFVATINWI |
| | PGSTYYADSVKGRFTISGDKSKNTVYLQMNSLRAEDTAVYYCAARRRIESYYRAS |
| | EYDYWGQGTLVTVSS |
| C09 | SEQ ID NO: 710 |
| Con | X₁X₂VQLX₃ESGGGLVQX₄GGSLRLSCAASGRTFSSYGMGWFRQAPGKEREFVAAIS |
| w/ | WIGX₅X₆X₇YYADSVKGRFTLSX₈DNX₉KNX₁₀X₁₁X₁₂LQMNSLX₁₃X₁₄EDTAVYYCA |
| D08, | AKYYX₁₅DSYX₁₆AAX₁₇SYAYWGQGTX₁₈VTVSS |
| E15, | X₁ is S, GG, AS, ASS or absent (i.e., no amino acid |
| F21 | residues ) |
| | X₂ is E or Q |
| | X₃ is L or Q |
| | X₄ is A or P |
| | X₅ is G or N |
| | X₆ is G or S |
| | X₇ is A or T |
| | X₈ is G, R or S |
| | X₉ is A or S |
| | X₁₀ is M, R or T |
| | X₁₁ is I, L or V |
| | X₁₂ is A or Y |
| | X₁₃ is K or R |
| | X₁₄ is A or P |
| | X₁₅ is P or T |
| | X₁₆ is F or Y |
| | X₁₇ is A or T |
| | X₁₈ is L or Q |
| C09 | SEQ ID NO: 224 |
| | X₁ is S, GG, AS, ASS or absent (i.e., no amino acid |
| | residues ) |
| | SEQ ID NO: 225 |
| | X₁ is AS |
| | X₁QVQLQESGGGLVQAGGSLRLSCAASGRTFSSYGMGWFRQAPGKEREFVAAISWI |
| | GGGTYYADSVKGRFTLSRDNAKNMVYLQMNSLKPEDTAVYYCAAKYYPDSYFAAA |
| | SYAYWGQGTQVTVSS |
| huC09 | SEQ ID NO: 226 |
| | EVQLLESGGGLVQPGGSLRLSCAASGRTFSSYGMGWFRQAPGKEREFVAAISWIG |
| | GGTYYADSVKGRFTLSRDNSKNTVYLQMNSLRAEDTAVYYCAAKYYPDSYFAAAS |
| | YAYWGQGTLVTVSS |
| D08 | SEQ ID NO: 227 |
| | X₁ is S, GG, AS, ASS or absent (i.e., no amino acid |
| | residues ) |
| | SEQ ID NO: 228 |
| | X₁ is AS |
| | X₁QVQLQESGGGLVQAGGSLRLSCAASGRTFSSYGMGWFRQAPGKEREFVAAISWI |
| | GGSTYYADSVKGRFTLSSDNAKNRIALQMNSLKPEDTAVYYCAAKYYTDSYFAAT |
| | SYAYWGQGTQVTVSS |
| huD08 | SEQ ID NO: 229 |
| | EVQLLESGGGLVQPGGSLRLSCAASGRTFSSYGMGWFRQAPGKEREFVAAISWIG |
| | GGTYYADSVKGRFTLSRDNSKNTVYLQMNSLRAEDTAVYYCAAKYYPDSYFAAAS |
| | YAYWGQGTLVTVSS |
| G22 | SEQ ID NO: 230 |
| con | X₁X₂LQLX₃ESGGGLVQX₄GGSLX₅LSCX₆X₇SGRTFSGYGMGWFRQX₈PGKEREFVS |
| w/ | X₉INWIYGATYYX₁₀DSVKGRFX₁₁X₁₂SX₁₃DKX₁₄KX₁₅TX₁₆YLQMX₁₇SLX₁₈X₁₉ED |
| D14, | TAVYYCAAKINGX₂₀SYFNX₂₁GSFGFWGQGTX₂₂VTVSS |
| L05, | X₁ is S, GG, AS, ASS or absent (i.e., no amino acid |
| L11 | residues ) |
| | X₂ is E or Q |
| | X₃ is L, Q or V |
| | X₄ is A or P |
| | X₅ is K or R |
| | X₆ is A, S or T |
| | X₇ is A or N |
| | X₈ is A or P |
| | X₉ is A or S |
| | X₁₀ is A, S or T |
| | X₁₁ is A or T |
| | X₁₂ is L or V |
| | X₁₃ is G or R |
| | X₁₄ is A, D or S |
| | X₁₅ is G or N |
| | X₁₆ is L, M or V |
| | X₁₇ is D, N or S |
| | X₁₈ is K or R |
| | X₁₉ is A or P |
| | X₂₀ is E or G |
| | X₂₁ is I or T |
| | X₂₂ is L or Q |
| D14 | SEQ ID NO: 231 |
| | ELQLVESGGGLVQAGGSLKLSCTASGRTFSGYGMGWFRQAPGKEREFVSSINWIY |
| | GATYYTDSVKGRFAVSRDKDKNTMYLQMSSLKPEDTAVYYCAAKINGGSYENTGS |
| | FGFWGQGTQVTVSS |
| huD14 | SEQ ID NO: 232 |
| | ELQLVESGGGLVQPGGSLRLSCAASGRTFSGYGMGWFRQAPGKEREFVSSINWIY |
| | GATYYTDSVKGRFTVSRDKSKNTMYLQMNSLRAEDTAVYYCAAKINGGSYENTGS |
| | FGFWGQGTLVTVSS |
| E07 | SEQ ID NO: 233 |
| con1 | X₁X₂LQLVESGGGLVX₃X₄GX₅SLRLSCAASGRTFSAYGMGWFRQAPGKX₆X₇EFVX₈ |
| | AIRWX₉GX₁₀X₁₁TYYAX₁₂SVKGRFTISKDX₁₃X₁₄KNTLYLQMX₁₅SLX₁₆X₁₇EDTAV |
| | YYCAAKTX₁₈X₁₉X₂₀SYLSEKSYX₂₁YWGQGTX₂₂VTVSS |
| | X₁ is S, GG, AS, ASS or absent (i.e., no amino acid |
| | residues ) |
| | X₂ is E or Q |
| | X₃ is Q or R |
| | X₄ is A or P |
| | X₅ is D or G |
| | X₆ is E or G |
| | X₇ is L or R |
| | X₈ is A or S |
| | X₉ is I or Y |
| | X₁₀ is D or E |
| | X₁₁ is A, G or S |
| | X₁₂ is D or E |
| | X₁₃ is N or S |
| | X₁₄ is A or S |
| | X₁₅ is N or S |
| | X₁₆ is K or R |
| | X₁₇ is A or P |
| | X₁₈ is R or S |
| | X₁₉ is P or V |
| | X₂₀ is D or T |
| | X₂₁ is G or N |
| | X₂₂ is M or Q |
| E07 | SEQ ID NO: 234 |
| con2 | X₁QLQLVESGGGLVX₃X₄GGSLRLSCAASGRTFSAYGMGWFRQAPGKX₆REFVX₇AI |
| | RWX₈GX₉X₁₀TYYAX₁₁SVKGRFTISKDNX₁₃KNTLYLQMX₁₄SLX₁₅X₁₆EDTAVYYCA |
| | AKTX₁₇X₁₈X₁₉SYLSEKSYX₂₀YWGQGTX₂₁VTVSS |
| | X₁ is S, GG, AS, ASS or absent (i.e., no amino acid |
| | residues ) |
| | X₃ is Q or R |
| | X₄ is A or P |
| | X₆ is E or G |
| | X₇ is A or S |
| | X₈ is I or Y |
| | X₉ is D or E |
| | X₁₀ is A, G or S |
| | X₁₁ is D or E |
| | X₁₃ is A or S |
| | X₁₄ is N or S |
| | X₁₅ is K or R |
| | X₁₆ is A or P |
| | X₁₇ is R or S |
| | X₁₈ is P or V |
| | X₁₉ is D or T |
| | X₂₀ is G or N |
| | X₂₁ is M or Q |
| E07 | SEQ ID NO: 235 |
| | X₁ is S, GG, AS, ASS or absent (i.e., no amino acid |
| | residues) |
| | SEQ ID NO: 236 |
| | X₁ is AS |
| | X₁QLQLVESGGGLVRAGGSLRLSCAASGRTFSAYGMGWFRQAPGKEREFVAAIRWI |
| | GDSTYYADSVKGRFTISKDNAKNTLYLQMSSLKPEDTAVYYCAAKTSVTSYLSEK |
| | SYNYWGQGTQVTVSS |
| huE07 | SEQ ID NO: 724 |
| | ELQLLESGGGLVQPGGSLRLSCAASGRTFSAYGMGWFRQAPGKEREFVAAIRWIG |
| | DSTYYADSVKGRFTISKDNSKNTLYLQMNSLRAEDTAVYYCAAKTSVTSYLSEKS |
| | YNYWGQGTMVTVSS |
| huE07 | SEQ ID NO: 725 |
| | ELQLLESGGGLVQPGGSLRLSCAASGRTFSAYGMGWFRQAPGKGREFVSAIRWIG |
| | DSTYYADSVKGRFTISKDNSKNTLYLQMNSLRAEDTAVYYCAAKTSVTSYLSEKS |
| | YNYWGQGTMVTVSS |
| huE07 | SEQ ID NO: 237 |
| | X₁ is S, GG, AS, ASS or absent (i.e., no amino acid |
| | residues) |
| | SEQ ID NO: 238 |
| | X₁ is AS |
| | X₁QLQLLESGGGLVQPGGSLRLSCAASGRTFSAYGMGWFRQAPGKGREFVSAIRWY |
| | GEGTYYAESVKGRFTISKDNSKNTLYLQMNSLRAEDTAVYYCAAKTRPDSYLSEK |
| | SYGYWGQGTMVTVSS |
| huE07 | SEQ ID NO: 239 |
| | X₁ is S, GG, AS, ASS or absent (i.e., no amino acid |
| | residues ) |
| | SEQ ID NO:240 |
| | X₁ is AS |
| | X₁QLQLLESGGGLVQPGGSLRLSCAASGRTFSAYGMGWFRQAPGKGREFVSAIRWI |
| | GDATYYAESVKGRFTISKDNSKNTLYLQMNSLRAEDTAVYYCAAKTSVTSYLSEK |
| | SYNYWGQGTMVTVSS |
| E15 | SEQ ID NO: 241 |
| | X₁ is S, GG, AS, ASS or absent (i.e., no amino acid |
| | residues ) |
| | SEQ ID NO: 242 |
| | X₁ is AS |
| | X₁QVQLQESGGGLVQAGGSLRLSCAASGRTFSSYGMGWFRQAPGKEREFVAAISWI |
| | GNSAYYADSVKGRFTLSGDNAKNTLYLQMNSLKPEDTAVYYCAAKYYTDSYYAAA |
| | SYAYWGQGTQVTVSS |
| huE15 | SEQ ID NO: 243 |
| | EVQLLESGGGLVQPGGSLRLSCAASGRTFSSYGMGWFRQAPGKEREFVAAISWIG |
| | NSAYYADSVKGRFTLSGDNSKNTLYLQMNSLRAEDTAVYYCAAKYYTDSYYAAAS |
| | YAYWGQGTLVTVSS |
| F09 | SEQ ID NO: 244 |
| | X₁ is S, GG, AS, ASS or absent (i.e., no amino acid |
| | residues ) |
| | SEQ ID NO: 245 |
| | X₁ is AS |
| | X₁QVQLQESGGGLVQAGGSLSLSCAASGRTFSSYGMGWFRQAPGKEREFVAAISWI |
| | GSATDYADSVRGRFTISGDNAKNTVYLLMNNLKPEDTAVYYCAARRSPMSRTSVL |
| | QYDYWGQGTQVTVSS |
| huF09 | SEQ ID NO: 246 |
| | EVQLLESGGGLVQPGGSLRLSCAASGRTFSSYGMGWFRQAPGKEREFVAAISWIG |
| | SATDYADSVRGRFTISGDNSKNTVYLQMNSLRAEDTAVYYCAARRSPMSRTSVLQ |
| | YDYWGQGTLVTVSS |
| F10 | SEQ ID NO: 247 |
| | EVQLVESGGGLVQAGDSLRLSCAASGRTFSAYGMGWFRQAPGKEREFVAAIRWIG |
| | DSTYYADSVKGRFTISKDSAKNTLYLQMSSLKPEDTAVYYCAAKTSVTSYLSEKA |
| | YNYWGQGTQVTVSS |
| huF10 | SEQ ID NO: 248 |
| | EVQLVESGGGLVQPGGSLRLSCAASGRTFSAYGMGWFRQAPGKEREFVAAIRWIG |
| | DSTYYADSVKGRFTISKDSSKNTLYLQMNSLRAEDTAVYYCAAKTSVTSYLSEKA |
| | YNYWGQGTMVTVSS |
| F11 | SEQ ID NO: 249 |
| con | EX₁QLX₂ESGGGLVQX₃GGSLRLSCAX₄SGRTFSSHTVGWFRQAPGKX₅REFVX₆AI |
| | TWNGDTTYFGDFVEGRFX₇ISRDX₈X₉KNTLYLX₁₀MNX₁₁LX₁₂X₁₃EDTAVYYCAAA |
| | TGQVWSLQYLRESSYAYWGQGTX₁₄VTVSS |
| | X₁ is L or V |
| | X₂ is L or V |
| | X₃ is A or P |
| | X₄ is A or N |
| | X₅ is E or G |
| | X₆ is A or E |
| | X₇ is S or T |
| | X₈ is N or S |
| | X₉ is A or S |
| | X₁₀ is E or Q |
| | X₁₁ is K or S |
| | X₁₂ is K or R |
| | X₁₃ is A or P |
| | X₁₄ is L or Q |
| F11 | SEQ ID NO: 250 |
| | ELQLVESGGGLVQAGGSLRLSCANSGRTFSSHTVGWFRQAPGKEREFVAAITWNG |
| | DTTYFGDFVEGRFSISRDSAKNTLYLEMNKLKPEDTAVYYCAAATGQVWSLQYLR |
| | ESSYAYWGQGTQVTVSS |
| huF11 | SEQ ID NO: 251 |
| | EVQLLESGGGLVQPGGSLRLSCAASGRTFSSHTVGWFRQAPGKGREFVAAITWNG |
| | DTTYFGDFVEGRETISRDNSKNTLYLQMNSLRAEDTAVYYCAAATGQVWSLQYLR |
| | ESSYAYWGQGTLVTVSS |
| huF11 | SEQ ID NO: 252 |
| | ELQLLESGGGLVQPGGSLRLSCAASGRTFSSHTVGWFRQAPGKGREFVSAITWNG |
| | DTTYFGDFVEGRFTISRDNSKNTLYLQMNSLRAEDTAVYYCAAATGQVWSLQYLR |
| | ESSYAYWGQGTLVTVSS |
| huF11 | SEQ ID NO: 752 |
| | EVQLLESGGGLVQPGGSLRLSCANSGRTFSSHTVGWFRQAPGKGREFVSAITWNG |
| | DTTYFGDFVEGRFTISRDNSKNTLYLQMNSLRAEDTAVYYCAAATGQVWSLQYLR |
| | ESSYAYWGQGTLVTVSS |
| huF11 | SEQ ID NO: 753 |
| | EVQLLESGGGLVQPGGSLRLSCAASGRTFSSHTVGWFRQAPGKGREFVSAITWNG |
| | DTTYFGDFVEGRFTISRDSSKNTLYLQMNSLRAEDTAVYYCAAATGQVWSLQYLR |
| | ESSYAYWGQGTLVTVSS |
| huF11 | SEQ ID NO: 754 |
| | EVQLLESGGGLVQPGGSLRLSCAASGRTFSSHTVGWFRQAPGKGREFVAAITWNG |
| | DTTYFGDFVEGRFTISRDNSKNTLYLQMNSLRAEDTAVYYCAAATGQVWSLQYLR |
| | ESSYAYWGQGTLVTVSS |
| huF11 | SEQ ID NO: 755 |
| | ELQLLESGGGLVQPGGSLRLSCAASGRTFSSHTVGWFRQAPGKGREFVSAITWNG |
| | DTTYFGDFVEGRFTISRDNSKNTLYLQMNSLRAEDTAVYYCAAATGQVWSLQYLR |
| | ESSYAYWGQGTLVTVSS |
| F21 | SEQ ID NO: 253 |
| | X₁ is S, GG, AS, ASS or absent (i.e., no amino acid |
| | residues ) |
| | SEQ ID NO: 254 |
| | X₁ is AS |
| | X₁QVQLVESGGGLVQAGGSLRLSCAASGRTFSSYGMGWFRQAPGKEREFVAAISWI |
| | GNSTYYADSVKGRFTLSGDNAKNTLYLQMNSLKPEDTAVYYCAAKYYTDSYYAAA |
| | SYAYWGQGTQVTVSS |
| huF21 | SEQ ID NO: 255 |
| | EVQLVESGGGLVQPGGSLRLSCAASGRTFSSYGMGWFRQAPGKEREFVAAISWIG |
| | NSTYYADSVKGRFTLSGDNSKNTLYLQMNSLRAEDTAVYYCAAKYYTDSYYAAAS |
| | YAYWGQGTLVTVSS |
| G22 | SEQ ID NO: 256 |
| | X₁ is S, GG, AS, ASS or absent (i.e., no amino acid |
| | residues) |
| | SEQ ID NO: 257 |
| | X₁ is AS |
| | X₁QLQLVESGGGLVQAGGSLKLSCTASGRTEDGYGMGWFRQAPGKEREFVSAINWI |
| | YGATYYADSVKGRFAVSRDKAKGTMYLQMNSLKPEDTAVYYCAAKINAGSYENIG |
| | SFGSWGQGTQVTVSS |
| huG22 | SEQ ID NO: 258 |
| | X₁ is S, GG, AS, ASS or absent (i.e., no amino acid |
| | residues ) |
| | SEQ ID NO: 259 |
| | X₁ is AS |
| | X₁QLQLVESGGGLVQPGGSLRLSCSASGRTEDGYGMGWFRQAPGKEREFVSAINWI |
| | YGATYYADSVKGRFTVSRDKSKNTMYLQMNSLRAEDTAVYYCAAKINAGSYFNIG |
| | SFGSWGQGTLVTVSS |
| G23 | SEQ ID NO: 260 |
| | X₁ is S, GG, AS, ASS or absent (i.e., no amino acid |
| | residues ) |
| | SEQ ID NO: 261 |
| | X₁ is AS |
| | X₁QLQLVESGGGLVQAGGSLRLSCAASGRTFSDDTVGWFRQAPGKEREFVAAITWN |
| | GDTTYYGDSVKGRFTISRDSAKNTLYLDMNKLEPEDTAVYYCAAAAGQDWSLGYL |
| | RESSYAYWGQGTQVTVSS |
| huG23 | SEQ ID NO: 262 |
| | X₁ is S, GG, AS, ASS or absent (i.e., no amino acid |
| | residues ) |
| | SEQ ID NO: 263 |
| | X₁ is AS |
| | X₁QLQLVESGGGLVQPGGSLRLSCSASGRTFSDDTVGWFRQAPGKEREFVAAITWN |
| | GDTTYYGDSVKGRFTISRDSSKNTLYLQMNSLRAEDTAVYYCAAAAGQDWSLGYL |
| | RESSYAYWGQGTLVTVSS |
| H22 | SEQ ID NO: 264 |
| | X₁ is S, GG, AS, ASS or absent (i.e., no amino acid |
| | residues ) |
| | SEQ ID NO: 265 |
| | X₁ is AS |
| | X₁QVQLQESGGGLVQAGDSLRLSCAASGRTFSGYTVGWFRQAPGKEREFVAAITWN |
| | GDTTYFGDSVKGRFTISRDSAKNTLYLEMNKLKPEDTAVYYCAAATGQEWSLQYL |
| | RESSYAYWGQGTQVTVSS |
| huH22 | SEQ ID NO: 266 |
| | EVQLLESGGGLVQPGGSLRLSCAASGRTFSGYTVGWFRQAPGKEREFVAAITWNG |
| | DTTYFGDSVKGRFTISRDSSKNTLYLQMNSLRAEDTAVYYCAAATGQEWSLQYLR |
| | ESSYAYWGQGTLVTVSS |
| I04 | SEQ ID NO: 267 |
| | X₁ is S, GG, AS, ASS or absent (i.e., no amino acid |
| | residues ) |
| | SEQ ID NO: 268 |
| | X₁ is AS |
| | X₁QVQLVESGGGLVQAGGSLRLSCAASGRTFSNYAMAWFRQAPGKEREFVSAINWF |
| | GRATEYADSVKDRETTSGDSAKNTVYLQMNSLKPEDTAVYYCAARTKVITGRRNQ |
| | AEYDYWGQGTQVTVSS |
| huI04 | SEQ ID NO: 269 |
| | EVQLVESGGGLVQPGGSLRLSCAASGRTFSNYAMAWFRQAPGKEREFVSAINWEG |
| | RATEYADSVKDRFTTSGDSSKNTVYLQMNSLRAEDTAVYYCAARTKVTTGRRNQA |
| | EYDYWGQGTLVTVSS |
| J13 | SEQ ID NO: 270 |
| | X₁ is S, GG, AS, ASS or absent (i.e., no amino acid |
| | residues ) |
| | SEQ ID NO: 271 |
| | X₁ is AS |
| | X₁QVQLVESGGGSVQAGGSLRLSCAASGRTFSSYGMGWFRQAPGKERGFVATINWI |
| | PGNTYYADSVKGRFTISGEEDKNTVYLQMDNLKPEDTAVYYCAARRRIDNYYRAS |
| | EYDYWGQGTQVTVSS |
| huJ13 | SEQ ID NO: 272 |
| | X₁ is S, GG, AS, ASS or absent (i.e., no amino acid |
| | residues ) |
| | SEQ ID NO: 273 |
| | X₁ is AS |
| | X₁QVQLVESGGGVVQPGGSLRLSCAASGRTFSSYGMGWFRQAPGKEREFVATINWI |
| | PGNTYYADSVKGRFTISGDESKNTVYLQMNSLRAEDTAVYYCAARRRIDNYYRAS |
| | EYDYWGQGTLVTVSS |
| J24 | SEQ ID NO: 274 |
| | X₁ is S, GG, AS, ASS or absent (i.e., no amino acid |
| | residues ) |
| | SEQ ID NO: 275 |
| | X₁ is AS |
| | X₁QVQLQESGGGSVQAGGSLRLSCAASGRTFSSYGMGWFRQAPGKERGEVATIIGW |
| | IPGNTYYADSVKGRFTITGDKDKNTVYLQMDNLKPEDTAVYYCAARRRIDSYYRA |
| | SEYDYWGQGTQVTVSS |
| huJ24 | SEQ ID NO: 276 |
| | X₁ is S, GG, AS, ASS or absent (i.e., no amino acid |
| | residues ) |
| | SEQ ID NO: 277 |
| | X₁ is AS |
| | X₁QVQLVESGGGVVQPGGSLRLSCAASGRTFSSYGMGWFRQAPGKEREFVATIIGW |
| | IPGNTYYADSVKGRFTISGDKSKNTVYLQMNSLRAEDTAVYYCAARRRIDSYYRA |
| | SEYDYWGQGTLVTVSS |
| L05 | SEQ ID NO: 278 |
| | X₁ is S, GG, AS, ASS or absent (i.e., no amino acid |
| | residues ) |
| | SEQ ID NO: 279 |
| | X₁ is AS |
| | X₁QVQLLESGGGLVQAGGSLKLSCTNSGRTFSGYGMGWFRQPPGKEREFVSSINWI |
| | YGATYYSDSVKGRFALSRDKAKNTVYLQMDSLKPEDTAVYYCAAKINGESYENTG |
| | SFGSWGQGTQVTVSS |
| huL05 | SEQ ID NO: 280 |
| | EVQLLESGGGLVQPGGSLRLSCAASGRTFSGYGMGWFRQAPGKEREFVSSINWIY |
| | GATYYSDSVKGRFTLSRDKSKNTVYLQMNSLRAEDTAVYYCAAKINGESYENTGS |
| | FGSWGQGTLVTVSS |
| L11 | SEQ ID NO: 281 |
| | X₁ is S, GG, AS, ASS or absent (i.e., no amino acid |
| | residues ) |
| | SEQ ID NO: 282 |
| | X₁ is AS |
| | X₁QLQLQESGGGLVQAGGSLKLSCTNSGRTFSGYGMGWFRQAPGKEREFVSAINWI |
| | YGATYYADSVKGRFAVSGDKAKNTLYLQMNSLKPEDTAVYYCAAKINGGSYENIG |
| | SFGSWGQGTQVTVSS |
| huL11 | SEQ ID NO: 283 |
| | X₁ is S, GG, AS, ASS or absent (i.e., no amino acid |
| | residues ) |
| | SEQ ID NO: 284 |
| | X₁ is AS |
| | X₁QLQLVESGGGLVQPGGSLRLSCSASGRTFSGYGMGWFRQAPGKEREFVSAINWI |
| | YGATYYADSVKGRFTVSGDKSKNTLYLQMNSLRAEDTAVYYCAAKINGGSYFNIG |
| | SFGSWGQGTLVTVSS |
| N16 | SEQ ID NO: 285 |
| | X₁ is S, GG, AS, ASS or absent (i.e., no amino acid |
| | residues ) |
| | SEQ ID NO: 286 |
| | X₁ is AS |
| | X₁QVQLVESGGGLVQAGGSLRLSCAASGGTFSYYGMGWFRQAPGKEREFVAAIRWI |
| | GGDTYYADSVKGRFTISKDNAKNTLYLQMSSLKPEDTAVYYCAAKTSVTSYLSEK |
| | SYNYWGQGTQVTVSS |
| huN16 | SEQ ID NO: 287 |
| | EVQLVESGGGLVQPGGSLRLSCAASGGTFSYYGMGWFRQAPGKEREFVAAIRWIG |
| | GDTYYADSVKGRFTISKDNSKNTLYLQMNSLRAEDTAVYYCAAKTSVTSYLSEKS |
| | YNYWGQGTLVTVSS |

In some embodiments, the VHH binds to an epitope on IL 10Rα comprising contact amino acid residues W33, E35, E37, H40, 142, H44, T46, L79, S80, T124, A170, R172, F178, F180, H182, Q204, K215, M217, W218, K220 and E221, wherein the amino acid residue positions are with respect to SEQ ID NO: 711 (e.g., VHH E07). In some embodiments, the VHH binds to an epitope on IL 10Rα comprising contact amino acid residues I169, I171, K173, F178, F180, T181, H182, K183, K186, F190, L192, L193, S195, E197 and F201, wherein the amino acid residue positions are with respect to SEQ ID NO: 711 (e.g., VHH F11).

| Human IL-10RA amino acid sequence - NP_001549.2/Uniprot Q13651 | |
|---|---|
| (SEQ ID NO: 711) | |
| 1 | MLPCLVVLLA ALLSLRLGSD AHGTELPSPP SVWFEAEFFH HILHWTPIPN QSESTCYEVA |
| 61 | LLRYGIESWN SISNCSQTLS YDLTAVILDL YHSNGYRARV RAVDGSRHSN WTVINTRFSV |
| 121 | DEVTLTVGSV NLEIHNGFIL GKIQLPRPKM APANDTYESI FSHFREYEIA IRKVPGNFTF |
| 181 | THKKVKHENF SLLTSGEVGE FCVQVKPSVA SRSNKGMWSK EECISLTRQY FTVINVIIFF |
| 241 | AFVLLLSGAL AYCLALQLYV RRRKKLPSVL LFKKPSPFIF ISQRPSPETQ DTIHPLDEEA |
| 301 | FLKVSPELKN LDLHGSTDSG FGSTKPSLQT EEPQFLLPDP HPQADRILGN REPPVLGDSC |
| 361 | SSGSSNSTDS GICLQEPSLS PSTGPTWEQQ VGSNSRGQDD SGIDLVQNSE GRAGDTQGGS |
| 421 | ALGHHSPPEP EVPGEEDPAA VAFQGYLRQT RCAEEKATKT GCLEEESPLT DGLGPKFGRC |
| 481 | LVDEAGLHPP ALAKGYLKQD PLEMTLASSG APTGQWNQPT EEWSLLALSS CSDLGISDWS |
| 541 | FAHDLAPLGC VAAPGGLLGS FNSDLVTLPL ISSLQSSE |

### 3. IL-10Rβ Single Immunoglobulin Variable Domain (VHH) Binders

Provided are single immunoglobulin variable domain (VHH) binders that specifically binds to the extracellular domain of interleukin-10 receptor (IL-10R) beta subunit (IL-10Rβ). Illustrative IL-10Rβ single immunoglobulin variable domain (VHH) binders are provided in Tables C1-C4 and D, below. The IL-10Rβ single immunoglobulin variable domain (VHH) binders can be humanized and incorporated into antibodies, including multispecific antibodies, e.g., bispecific antibodies, e.g., comprising a human Fc domain, e.g., a human IgG1 or a human IgG4. The IL-10Rβ single immunoglobulin variable domain (VHH) binders described herein are suitable to use with an antigen binding domain that specifically binds to the extracellular domain of interleukin-10 receptor (IL-10R) alpha subunit (IL 10Rα), including the IL-10Rα single immunoglobulin variable domain (VHH) binders described herein.

In some embodiments, the VHH binds to IL 10Rβ with an equilibrium dissociation constant (K_{D}) of greater than of greater than about 10 nM, e.g., greater than about 20 nM, greater than about 30 nM, greater than about 40 nM or greater than about 50 nM.

In some embodiments, the VHH comprises a complementarity determining region (CDR) 1, a VHH-CDR2, and a VHH-CDR3 comprising the following amino acid sequences, according to Kabat, respectively:
1) SEQ ID NOs: 288, 289 and 290; (A02)
2) SEQ ID NOs: 291, 292 and 293; (A05)
3) SEQ ID NOs: 291, 294 and 295; (huA05)
4) SEQ ID NOs: 296, 297 and 298; (huA05)
5) SEQ ID NOs: 299, 300 and 301; (B10)
6) SEQ ID NOs: 302, 303 and 304; (B12)
7) SEQ ID NOs: 305, 306 and 307; (C01)
8) SEQ ID NOs: 296, 308 and 309; (C04)
9) SEQ ID NOs: 310, 311 and 312; (C10)
10) SEQ ID NOs: 302, 303 and 313; (D01)
11) SEQ ID NOs: 314, 315 and 316; (E08)
12) SEQ ID NOs: 317, 318 and 319; (E10)
13) SEQ ID NOs: 320, 321 and 322; (G01) or
14) SEQ ID NOs: 323, 324 and 325 (G02).

In some embodiments, the VHH comprises a complementarity determining region (CDR) 1, a VHH-CDR2, and a VHH-CDR3 comprising the following amino acid sequences, according to IMGT, respectively:
1) SEQ ID NOs: 326, 327 and 328; (A02)
2) SEQ ID NOs: 329, 330 and 331; (A05)
3) SEQ ID NOs: 329, 332 and 333; (huA05)
4) SEQ ID NOs: 334, 335 and 336; (huA05)
5) SEQ ID NOs: 337, 338 and 339; (B10)
6) SEQ ID NOs: 340, 341 and 342; (B12)
7) SEQ ID NOs: 343, 344 and 345; (C01)
8) SEQ ID NOs: 346, 347 and 348; (C04)
9) SEQ ID NOs: 349, 350 and 351; (C10)
10) SEQ ID NOs: 352, 341 and 353; (D01)
11) SEQ ID NOs: 354, 355 and 356; (E08)
12) SEQ ID NOs: 357, 358 and 359; (E10)
13) SEQ ID NOs: 360, 361 and 362; (G01) or
14) SEQ ID NOs: 363, 364 and 365 (G02).

In some embodiments, the VHH comprises a complementarity determining region (CDR) 1, a VHH-CDR2, and a VHH-CDR3 comprising the following amino acid sequences, according to Chothia, respectively:
1) SEQ ID NOs: 366, 367 and 368; (A02)
2) SEQ ID NOs: 369, 370 and 371; (A05)
3) SEQ ID NOs: 369, 372 and 373; (huA05)
4) SEQ ID NOs: 374, 375 and 376; (huA05)
5) SEQ ID NOs: 377, 378 and 379; (B10)
6) SEQ ID NOs: 380, 381 and 382; (B12)
7) SEQ ID NOs: 383, 384 and 385; (C01)
8) SEQ ID NOs: 386, 387 and 388; (C04)
9) SEQ ID NOs: 389, 390 and 391; (C10)
10) SEQ ID NOs: 392, 381 and 393; (D01)
11) SEQ ID NOs: 394, 395 and 396; (E08)
12) SEQ ID NOs: 397, 398 and 399; (E10)
13) SEQ ID NOs: 400, 401 and 402; (G01) or
14) SEQ ID NOs: 403, 404 and 405 (G02).

In some embodiments, the VHH comprises a complementarity determining region (CDR) 1, a VHH-CDR2, and a VHH-CDR3 comprising the following amino acid sequences, according to Honegger, respectively:
1) SEQ ID NOs: 406, 407 and 408; (A02)
2) SEQ ID NOs: 409, 410 and 411; (A05)
3) SEQ ID NOs: 409, 412 and 413; (huA05)
4) SEQ ID NOs: 414, 415 and 416; (huA05)
5) SEQ ID NOs: 417, 418 and 419; (B10)
6) SEQ ID NOs: 420, 421 and 422; (B12)
7) SEQ ID NOs: 423, 424 and 425; (C01)
8) SEQ ID NOs: 426, 427 and 428; (C04)
9) SEQ ID NOs: 429, 430 and 431; (C10)
10) SEQ ID NOs: 432, 421 and 433; (D01)
11) SEQ ID NOs: 434, 435 and 436; (E08)
12) SEQ ID NOs: 437, 438 and 439; (E10)
13) SEQ ID NOs: 440, 441 and 442; (G01) or
14) SEQ ID NOs: 443, 444 and 445 (G02).

In some embodiments, the VHH comprises the amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 448, 449, 455 and 460. In some embodiments, the VHH comprises the amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 446-447, 450-454, 456-459 and 461-474.

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 288, 289 and 290 (according to Kabat);
2) SEQ ID NOs: 326, 327 and 328 (according to IMGT);
3) SEQ ID NOs: 366, 367 and 368 (according to Chothia); or
4) SEQ ID NOs: 406, 407 and 408 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth below, or an amino acid sequence at least 90%, 91%, 92%, 93%, 94%, 95%, 96%, 97%, 98%, 99% identical to, respectively, the full length of the amino acid sequence set forth in any one of SEQ ID NOs: 446-447 (VHH A02).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 291, 292 and 293; or 291, 294 and 295; or 296, 297 and 298 (according to Kabat);
2) SEQ ID NOs: 329, 330 and 331; or 329, 332 and 333; or 334, 335 and 336 (according to IMGT);
3) SEQ ID NOs: 369, 370 and 371; or 369, 372 and 373; or 374, 375 and 376 (according to Chothia); or
4) SEQ ID NOs: 409, 410 and 411; 409, 412 and 413; or 414, 415 and 416 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth below, or an amino acid sequence at least 90%, 91%, 92%, 93%, 94%, 95%, 96%, 97%, 98%, 99% identical to, respectively, the full length of the amino acid sequence set forth in any one of SEQ ID NOs: 448-452 (VHH A05).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 299, 300 and 301 (according to Kabat);
2) SEQ ID NOs: 337, 338 and 339 (according to IMGT);
3) SEQ ID NOs: 377, 378 and 379 (according to Chothia); or
4) SEQ ID NOs: 417, 418 and 419 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 453-454, or an amino acid sequence at least 90%, 91%, 92%, 93%, 94%, 95%, 96%, 97%, 98%, 99% identical to, respectively, the full length of the amino acid sequence set forth in any one of SEQ ID NOs: 453-454 (VHH B10).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 302, 303 and 304 (according to Kabat);
2) SEQ ID NOs: 340, 341 and 342 (according to IMGT);
3) SEQ ID NOs: 380, 381 and 382 (according to Chothia); or
4) SEQ ID NOs: 420, 421 and 422 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 455-457, or an amino acid sequence at least 90%, 91%, 92%, 93%, 94%, 95%, 96%, 97%, 98%, 99% identical to, respectively, the full length of the amino acid sequence set forth in any one of SEQ ID NOs: 455-457 (VHH B12).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 305, 306 and 307 (according to Kabat);
2) SEQ ID NOs: 343, 344 and 345 (according to IMGT);
3) SEQ ID NOs: 383, 384 and 385 (according to Chothia); or
4) SEQ ID NOs: 423, 424 and 425 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 458-459, or an amino acid sequence at least 90%, 91%, 92%, 93%, 94%, 95%, 96%, 97%, 98%, 99% identical to, respectively, the full length of the amino acid sequence set forth in any one of SEQ ID NOs: 458-459 (VHH C01).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 296, 308 and 309 (according to Kabat);
2) SEQ ID NOs: 346, 347 and 348 (according to IMGT);
3) SEQ ID NOs: 386, 387 and 388 (according to Chothia); or
4) SEQ ID NOs: 426, 427 and 428 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 460-462, or an amino acid sequence at least 90%, 91%, 92%, 93%, 94%, 95%, 96%, 97%, 98%, 99% identical to, respectively, the full length of the amino acid sequence set forth in any one of SEQ ID NOs: 460-462 (VHH C04).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 310, 311 and 312 (according to Kabat);
2) SEQ ID NOs: 349, 350 and 351 (according to IMGT);
3) SEQ ID NOs: 389, 390 and 391 (according to Chothia); or
4) SEQ ID NOs: 429, 430 and 431 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 463-464, or an amino acid sequence at least 90%, 91%, 92%, 93%, 94%, 95%, 96%, 97%, 98%, 99% identical to, respectively, the full length of the amino acid sequence set forth in any one of SEQ ID NOs: 463-464 (VHH C10).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 302, 303 and 313 (according to Kabat);
2) SEQ ID NOs: 352, 341 and 353 (according to IMGT);
3) SEQ ID NOs: 392, 381 and 393 (according to Chothia); or
4) SEQ ID NOs: 432, 421 and 433 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 455 and 465-466, or an amino acid sequence at least 90%, 91%, 92%, 93%, 94%, 95%, 96%, 97%, 98%, 99% identical to, respectively, the full length of the amino acid sequence set forth in any one of SEQ ID NOs: 455 and 465-466 (VHH D01).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 314, 315 and 316 (according to Kabat);
2) SEQ ID NOs: 354, 355 and 356 (according to IMGT);
3) SEQ ID NOs: 394, 395 and 396 (according to Chothia); or
4) SEQ ID NOs: 434, 435 and 436 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 467-468, or an amino acid sequence at least 90%, 91%, 92%, 93%, 94%, 95%, 96%, 97%, 98%, 99% identical to, respectively, the full length of the amino acid sequence set forth in any one of SEQ ID NOs: 467-468 (VHH E08).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 317, 318 and 319 (according to Kabat);
2) SEQ ID NOs: 357, 358 and 359 (according to IMGT);
3) SEQ ID NOs: 397, 398 and 399 (according to Chothia); or
4) SEQ ID NOs: 437, 438 and 439 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 469-470, or an amino acid sequence at least 90%, 91%, 92%, 93%, 94%, 95%, 96%, 97%, 98%, 99% identical to, respectively, the full length of the amino acid sequence set forth in any one of SEQ ID NOs: 469-470 (VHH E10).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 320, 321 and 322 (according to Kabat);
2) SEQ ID NOs: 360, 361 and 362 (according to IMGT);
3) SEQ ID NOs: 400, 401 and 402 (according to Chothia); or
4) SEQ ID NOs: 440, 441 and 442 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 471-472, or an amino acid sequence at least 90%, 91%, 92%, 93%, 94%, 95%, 96%, 97%, 98%, 99% identical to, respectively, the full length of the amino acid sequence set forth in any one of SEQ ID NOs: 471-472 (VHH G01).

In some embodiments, provided is a single domain antibody (sdAb) or antigen-binding fragment thereof (VHH) that specifically binds to IL-10Rβ comprising a heavy chain variable region (VH)-complementarity determining region (CDR) 1; a VH-CDR2, a VH-CDR3, a light chain variable region (VL)-CDR1, a VL-CDR2, and a VL-CDR3 comprising, respectively, the amino acid sequences of:
1) SEQ ID NOs: 323, 324 and 325 (according to Kabat);
2) SEQ ID NOs: 363, 364 and 365 (according to IMGT);
3) SEQ ID NOs: 403, 404 and 405 (according to Chothia); or
4) SEQ ID NOs: 443, 444 and 445 (according to Honegger).
   In some embodiments, provided is a single domain antibody (sdAb) or antigen-binding fragment thereof (VHH) that specifically binds to IL-10Rβ, wherein the VH and VL comprise, respectively, the amino acid sequences set forth in any one of SEQ ID NOs: 473-474, or an amino acid sequence at least 90%, 91%, 92%, 93%, 94%, 95%, 96%, 97%, 98%, 99% identical to, respectively, the full length of the amino acid sequence set forth in any one of SEQ ID NOs: 473-474 (VHH G02).

Generally, the herein described VHH that bind to IL-10Rβ bind to both human IL-10Rβ and cynomolgus IL-10Rβ, within a 10-fold affinity difference, e.g., within a 5-fold or within a 3-fold affinity difference (e.g., as determined by SPR and expressed as an equilibrium dissociation constant (KD)). Amino acid sequences of complementarity determining regions (CDRs) of illustrative VHH that bind to IL-10Rβ are provided in Tables C1-C4. Amino acid sequences of variable regions of illustrative VHH that bind to IL-10Rβ are provided in Table D.

TABLE C1
Illustrative IL-10Rβ Single Immunoglobulin Variable
domain (VHH) Binder CDR amino acid sequences
(according to Kabat)

| VHH | | | |
|---|---|---|---|
| name | VHH - CDR1 | VHH - CDR2 | VHH - CDR3 |
| A02 | EGVIG | CISARDGSTHYANAVKG | SLWGLGCKREEDYDY |
| | SEQ ID NO: 288 | SEQ ID NO: 289 | SEQ ID NO: 290 |
| A05 | YPTIG | CISASDGSTHYADSVKG | SLWGLGCKSEADYDY |
| | SEQ ID NO: 291 | SEQ ID NO: 292 | SEQ ID NO: 293 |
| huA05 | YPTIG | AISASDASTHYADAVKG | SLHGLGAKSEADYDY |
| | SEQ ID NO: 291 | SEQ ID NO: 294 | SEQ ID NO: 295 |
| huA05 | YSTIG | CISASEGSTHYADAVKG | SLHGLGCKSEADYDY |
| | SEQ ID NO: 296 | SEQ ID NO: 297 | SEQ ID NO: 298 |
| B10 | LDHMG | YMFTDGRTQYGEPVKG | QGI |
| | SEQ ID NO: 299 | SEQ ID NO: 300 | SEQ ID NO: 301 |
| B12 | ISTMA | RIIWSSGATNYADSVKG | AISQTRGTFWTY |
| | SEQ ID NO: 302 | SEQ ID NO: 303 | SEQ ID NO: 304 |
| C01 | SYGMS | QINTGGGSTSYADSVKG | SSRLYSSSWYGSREYDY |
| | SEQ ID NO: 305 | SEQ ID NO: 306 | SEQ ID NO: 307 |
| C04 | YSTIG | CISSSGRGVNANYIDSVRG | NCWRLTSPAADN |
| | SEQ ID NO: 296 | SEQ ID NO: 308 | SEQ ID NO: 309 |
| C10 | YYSIS | CISRSGGRINYADSVKG | TRLPLSGLCVIGSSDNAL |
| | SEQ ID NO: 310 | SEQ ID NO: 311 | SEQ ID NO: 312 |
| D01 | ISTMA | RIIWSSGATNYADSVKG | AISQRSGTFWTY |
| | SEQ ID NO: 302 | SEQ ID NO: 303 | SEQ ID NO: 313 |
| E08 | GYAVA | SISSSGIRTNYKDSVKG | KRGPILFGTYTH |
| | SEQ ID NO: 314 | SEQ ID NO: 315 | SEQ ID NO: 316 |
| E10 | IFAMG | AVTLRSGITSYLDSVKG | RDYRSRDY |
| | SEQ ID NO: 317 | SEQ ID NO: 318 | SEQ ID NO: 319 |
| G01 | RYAIG | CITTSGGGSNYADSVKG | TP LNYYCGIDENPDKYDY |
| | SEQ ID NO: 320 | SEQ ID NO: 321 | SEQ ID NO: 322 |
| G02 | FAPMG | AINRDFSTYYANSVKG | SPGRIWSGTILPPAPDRVVS |
| | SEQ ID NO: 323 | SEQ ID NO: 324 | SEQ ID NO: 325 |

TABLE C2
Illustrative IL-10Rβ Single Immunoglobulin
Variable domain (VHH) Binder CDR amino acid sequences
(according to IMGT)

| VHH | | | |
|---|---|---|---|
| name | VHH - CDR1 | VHH - CDR2 | VHH - CDR3 |
| A02 | GISLDEGV | ISARDGST | AASLWGLGCKREEDYDY |
| | SEQ ID NO: 326 | SEQ ID NO: 327 | SEQ ID NO: 328 |
| A05 | GNSLDYPT | ISASDGST | AASLWGLGCKSEADYDY |
| | SEQ ID NO: 329 | SEQ ID NO: 330 | SEQ ID NO: 331 |
| huA05 | GNSLDYPT | ISASDAST | AASLHGLGAKSEADYDY |
| | SEQ ID NO: 329 | SEQ ID NO: 332 | SEQ ID NO: 333 |
| huA05 | GNSLDYST | ISASEGST | AASLHGLGCKSEADYDY |
| | SEQ ID NO: 334 | SEQ ID NO: 335 | SEQ ID NO: 336 |
| B10 | GSTSTLDH | MFTDGRT | GAQGI |
| | SEQ ID NO: 337 | SEQ ID NO: 338 | SEQ ID NO: 339 |
| B12 | GSFSSIST | IIWSSGAT | SGAISQTRGTFWTY |
| | SEQ ID NO: 340 | SEQ ID NO: 341 | SEQ ID NO: 342 |
| C01 | GFAFSSYG | INTGGGST | AASSRLYSSSWYGSREYDY |
| | SEQ ID NO: 343 | SEQ ID NO: 344 | SEQ ID NO: 345 |
| C04 | GFTSPYST | ISSSGRGVNA | ATNCWRLTSPAADN |
| | SEQ ID NO: 346 | SEQ ID NO: 347 | SEQ ID NO: 348 |
| C10 | GLTLDYYS | ISRSGGRI | AATRLPLSGLCVIGSSDNAL |
| | SEQ ID NO: 349 | SEQ ID NO: 350 | SEQ ID NO: 351 |
| D01 | GNFSSIST | IIWSSGAT | AGAISQRSGTFWTY |
| | SEQ ID NO: 352 | SEQ ID NO: 341 | SEQ ID NO: 353 |
| E08 | ISTLNGYA | ISSSGIRT | AAKRGP ILFGTYTH |
| | SEQ ID NO: 354 | SEQ ID NO: 355 | SEQ ID NO: 356 |
| E10 | GSALSIFA | VTLRSGIT | NVRDYRSRDY |
| | SEQ ID NO: 357 | SEQ ID NO: 358 | SEQ ID NO: 359 |
| G01 | GFTVDRYA | ITTSGGGS | AATPLNYYCGIDENPDKYDY |
| | SEQ ID NO: 360 | SEQ ID NO: 361 | SEQ ID NO: 362 |
| G02 | ETIINFAP | INRDEST | AASPGRIWSGTILPPAPDRVVS |
| | SEQ ID NO: 363 | SEQ ID NO: 364 | SEQ ID NO: 365 |

TABLE C3
Illustrative IL-10Rβ Single Immunoglobulin
Variable domain (VHH) Binder CDR amino acid sequences
(according to Chothia)

| VHH | | | |
|---|---|---|---|
| name | VHH - CDR1 | VHH - CDR2 | VHH - CDR3 |
| A02 | GISLDEG | ARDG | LWGLGCKREEDYD |
| | SEQ ID NO: 366 | SEQ ID NO: 367 | SEQ ID NO: 368 |
| A05 | GNSLDYP | ASDG | LWGLGCKSEADYD |
| | SEQ ID NO: 369 | SEQ ID NO: 370 | SEQ ID NO: 371 |
| huA05 | GNSLDYP | ASDA | LHGLGAKSEADYD |
| | SEQ ID NO: 369 | SEQ ID NO: 372 | SEQ ID NO: 373 |
| huA05 | GNSLDYS | ASEG | LHGLGCKSEADYD |
| | SEQ ID NO: 374 | SEQ ID NO: 375 | SEQ ID NO: 376 |
| B10 | GSTSTLD | TDG | G |
| | SEQ ID NO: 377 | SEQ ID NO: 378 | SEQ ID NO: 379 |
| B12 | GSFSSIS | WSSG | ISQTRGTFWT |
| | SEQ ID NO: 380 | SEQ ID NO: 381 | SEQ ID NO: 382 |
| C01 | GFAFSSY | TGGG | SRLYSSSWYGSREYD |
| | SEQ ID NO: 383 | SEQ ID NO: 384 | SEQ ID NO: 385 |
| C04 | GFTSPYS | SSGRGV | CWRLTSPAAD |
| | SEQ ID NO: 386 | SEQ ID NO: 387 | SEQ ID NO: 388 |
| C10 | GLTLDYY | RSGG | RLPLSGLCVIGSSDNA |
| | SEQ ID NO: 389 | SEQ ID NO: 390 | SEQ ID NO: 391 |
| D01 | GNESSIS | WSSG | ISQRSGTFWT |
| | SEQ ID NO: 392 | SEQ ID NO: 381 | SEQ ID NO: 393 |
| E08 | ISTLNGY | SSGI | RGP ILFGTYT |
| | SEQ ID NO: 394 | SEQ ID NO: 395 | SEQ ID NO: 396 |
| E10 | GSALSIF | LRSG | DYRSRD |
| | SEQ ID NO: 397 | SEQ ID NO: 398 | SEQ ID NO: 399 |
| G01 | GFTVDRY | TSGG | PLNYYCGIDENPDKYD |
| | SEQ ID NO: 400 | SEQ ID NO: 401 | SEQ ID NO: 402 |
| G02 | ETIINFA | RDF | PGRIWSGTILPPAPDRVV |
| | SEQ ID NO: 403 | SEQ ID NO: 404 | SEQ ID NO: 405 |

TABLE C4
Illustrative IL-10Rβ Single Immunoglobulin Variable
domain (VHH) Binder CDR amino acid sequences
(according to Honegger)

| VHH | | | |
|---|---|---|---|
| name | VHH - CDR1 | VHH - CDR2 | VHH - CDR3 |
| A02 | ASGISLDEGV | ISARDGSTHYANAVKGR | SLWGLGCKREEDYD |
| | SEQ ID NO: 406 | SEQ ID NO: 407 | SEQ ID NO: 408 |
| A05 | ASGNSLDYPT | ISASDGSTHYADSVKGR | SLWGLGCKSEADYD |
| | SEQ ID NO: 409 | SEQ ID NO: 410 | SEQ ID NO: 411 |
| huA05 | ASGNSLDYPT | ISASDASTHYADAVKGR | SLHGLGAKSEADYD |
| | SEQ ID NO: 409 | SEQ ID NO: 412 | SEQ ID NO: 413 |
| huA05 | ASGNSLDYST | ISASEGSTHYADAVKGR | SLHGLGCKSEADYD |
| | SEQ ID NO: 414 | SEQ ID NO: 415 | SEQ ID NO: 416 |
| B10 | ASGSTSTLDH | MFTDGRTQYGEPVKGR | QG |
| | SEQ ID NO: 417 | SEQ ID NO: 418 | SEQ ID NO: 419 |
| B12 | ASGSFSSIST | IIWSSGATNYADSVKGR | AISQTRGTFWT |
| | SEQ ID NO: 420 | SEQ ID NO: 421 | SEQ ID NO: 422 |
| C01 | ASGFAFSSYG | INTGGGSTSYADSVKGR | SSRLYSSSWYGSREYD |
| | SEQ ID NO: 423 | SEQ ID NO: 424 | SEQ ID NO: 425 |
| C04 | ASGFTSPYST | ISSSGRGVNANYIDSVRGR | NCWRLTSPAAD |
| | SEQ ID NO: 426 | SEQ ID NO: 427 | SEQ ID NO: 428 |
| C10 | ASGLTLDYYS | ISRSGGRINYADSVKGR | TRLPLSGLCVIGSSDNA |
| | SEQ ID NO: 429 | SEQ ID NO: 430 | SEQ ID NO: 431 |
| D01 | ASGNESSIST | IIWSSGATNYADSVKGR | AISQRSGTFWT |
| | SEQ ID NO: 432 | SEQ ID NO: 421 | SEQ ID NO: 433 |
| E08 | SSISTLNGYA | ISSSGIRTNYKDSVKGR | KRGP ILFGTYT |
| | SEQ ID NO: 434 | SEQ ID NO: 435 | SEQ ID NO: 436 |
| E10 | ASGSALSIFA | VTLRSGITSYLDSVKGR | RDYRSRD |
| | SEQ ID NO: 437 | SEQ ID NO: 438 | SEQ ID NO: 439 |
| G01 | ASGFTVDRYA | ITTSGGGSNYADSVKGR | TPLNYYCGIDENPDKYD |
| | SEQ ID NO: 440 | SEQ ID NO: 441 | SEQ ID NO: 442 |
| G02 | ASETIINFAP | INRDFSTYYANSVKGR | SPGRIWSGTILPPAPDRVV |
| | SEQ ID NO: 443 | SEQ ID NO: 444 | SEQ ID NO: 445 |

TABLE D
Illustrative IL-10Rβ Single Immunoglobulin Variable
Domain (VHH) amino acid sequences

| VHH | |
|---|---|
| name | VHH amino acid sequence |
| A02 | SEQ ID NO: 446 |
| | QVQLQESGGGLVQPGGSLKLSCAASGISLDEGVIGWERRAPGKEREGVSCISARDG |
| | STHYANAVKGRFTISRDNTKNTVDLQMNNLEPEDAGRYYCAASLWGLGCKREEDYD |
| | YWGQGTQVTVSS |
| huA02 | SEQ ID NO: 447 |
| | EVQLLESGGGLVQPGGSLRLSCAASGISLDEGVIGWFRQAPGKEREGVSCISARDG |
| | STHYANAVKGRFTISRDNSKNTVYLQMNSLRAEDTAVYYCAASLWGLGCKREEDYD |
| | YWGQGTLVTVSS |
| A05 | SEQ ID NO: 448 |
| con1 | X₁VQLX₂ESGGGLVQPGGSLX₃LSCAASGX₄SLDX₅X₆X₇IGWFRX₈APGKX₉X₁₀EGVS |
| | X₁₁ISAX₁₂DX₁₃STHYAX₁₄X₁₅VKGRFTISRDNX₁₆KNTVX₁₇LQMNX₁₈LX₁₉X₂₀ED |
| | X₂₁X₂₂X₂₃YYCAASLX₂₄GLGX₂₅KX₂₆EX₂₇DYDYWGQGTX₂₈VTVSS |
| | X₁ is E or Q |
| | X₂ is L or Q |
| | X₃ is K or R |
| | X₄ is I or N |
| | X₅ is E or Y |
| | X₆ is G, P or S |
| | X₇ is V or T |
| | X₈ is Q or R |
| | X₉ is E or G |
| | X₁₀ is L or R |
| | X₁₁ is A or C |
| | X₁₂ is R or S |
| | X₁₃ is A or G |
| | X₁₄ is D or N |
| | X₁₅ is A or S |
| | X₁₆ is S or T |
| | X₁₇ is D or Y |
| | X₁₈ is N or S |
| | X₁₉ is K or R |
| | X₂₀ is A or P |
| | X₂₁ is A or T |
| | X₂₂ is A or G |
| | X₂₃ is H or V |
| | X₂₄ is H or W |
| | X₂₅ is A or C |
| | X₂₆ is R or S |
| | X₂₇ is A or E |
| | X₂₈ is L, M or Q |
| A05 | SEQ ID NO: 449 |
| con2 | X₁VQLVESGGGLVQPGGSLRLSCAASGNSLDYPTIGWFRQAPGKX₂X₃EGVSX₄ISA |
| | SDX₅STHYADX₆VKGRFTISRDNX₇KNTVX₈LQMNX₉LX₁₀X₁₁EDTX₁₂X₁₃YYCAASL |
| | X₁₄GLGX₁₅KSEADYDYWGQGTX₁₆VTVSS |
| | X₁ is E or Q |
| | X₂ is E or G |
| | X₃ is L or R |
| | X₄ is A or C |
| | X₅ is A or G |
| | X₆ is A or S |
| | X₇ is S or T |
| | X₈ is D or Y |
| | X₉ is N or S |
| | X₁₀ is K or R |
| | X₁₁ is A or P |
| | X₁₂ is A or G |
| | X₁₃ is H or V |
| | X₁₄ is H or W |
| | X₁₅ is A or C |
| | X₁₆ is M or Q |
| A05 | SEQ ID NO: 450 |
| | QVQLVESGGGLVQPGGSLRLSCAASGNSLDYPTIGWFRQAPGKEREGVSCISASDG |
| | STHYADSVKGRFTISRDNTKNTVDLQMNNLKPEDTGHYYCAASLWGLGCKSEADYD |
| | YWGQGTQVTVSS |
| huA05 | SEQ ID NO: 451 |
| | EVQLVESGGGLVQPGGSLRLSCAASGNSLDYPTIGWFRQAPGKGLEGVSAISASDA |
| | STHYADAVKGRFTISRDNSKNTVYLQMNSLRAEDTAVYYCAASLHGLGAKSEADYD |
| | YWGQGTMVTVSS |
| huA05 | SEQ ID NO: 452 |
| | EVQLVESGGGLVQPGGSLRLSCAASGNSLDYSTIGWFRQAPGKGLEGVSCISASEG |
| | STHYADAVKGRFTISRDNSKNTVYLQMNSLRAEDTAVYYCAASLHGLGCKSEADYD |
| | YWGQGTMVTVSS |
| B10 | SEQ ID NO: 453 |
| | QVQLVESGGGVVQAGGSLRLSCAASGSTSTLDHMGWYRQAPGKSRELVGYMFTDGR |
| | TQYGEPVKGRFTVSRDNGRNTIFLQMNNLKLEDTAVYYCGAQGIWGQGTQVTVSS |
| huB10 | SEQ ID NO: 454 |
| | QVQLVESGGGVVQPGGSLRLSCAASGSTSTLDHMGWYRQAPGKSRELVGYMFTDGR |
| | TQYGEPVKGRFTVSRDNSKNTIYLQMNSLRAEDTAVYYCGAQGIWGQGTMVTVSS |
| B12 | SEQ ID NO: 455 |
| Con | X₁VQLX₂ESGGGLVX₃X₄GGSLRLSCAASGX₅FSSISTMAWYRQX₆PGKQREWX₇ARI |
| w / | IWSSGATNYADSVKGRFTX₈SRDNX₉KNTMYLQMX₁₀X₁₁LX₁₂X₁₃EDTAAYX₁₄CSGA |
| D01 | ISQX₁₅RGTFWTYX₁₆SQGTX₁₇VTVSS |
| | X₁ is E or Q |
| | X₂ is L, Q or V |
| | X₃ is K or Q |
| | X₄ is A or P |
| | X₅ is N or S |
| | X₆ is A or V |
| | X₇ is L or V |
| | X₈ is A or I |
| | X₉ is A, S or T |
| | X₁₀ is D or N |
| | X₁₁ is R or S |
| | X₁₂ is R or T |
| | X₁₃ is A or P |
| | X₁₄ is L or Y |
| | X₁₅ is R or T |
| | X₁₆ is K or W |
| | X₁₇ is L or Q |
| B12 | SEQ ID NO: 456 |
| | EVQLVESGGGLVQAGGSLRLSCAASGSFSSISTMAWYRQVPGKQREWLARIIWSSG |
| | ATNYADSVKGRFTISRDNTKNTMYLQMDRLTPEDTAAYLCSGAISQTRGTFWTYKS |
| | QGTQVTVSS |
| huB12 | SEQ ID NO: 457 |
| | EVQLVESGGGLVQPGGSLRLSCAASGSFSSISTMAWYRQAPGKQREWLARIIWSSG |
| | ATNYADSVKGRFTISRDNSKNTMYLQMNSLRAEDTAVYYCSGAISQTRGTFWTYKG |
| | QGTLVTVSS |
| C01 | SEQ ID NO: 458 |
| | QVQLQESGGGLVQPGGSLRLSCAASGFAFSSYGMSWVRQAPGKGLEWVSQINTGGG |
| | STSYADSVKGRFTISRDNAKNTLYLQMNSLKPEDTAVYYCAASSRLYSSSWYGSRE |
| | YDYWGQGTQVTVSS |
| huC01 | SEQ ID NO: 459 |
| | EVQLLESGGGLVQPGGSLRLSCAASGFAFSSYGMSWVRQAPGKGLEWVSQINTGGG |
| | STSYADSVKGRFTISRDNSKNTLYLQMNSLRAEDTAVYYCAASSRLYSSSWYGSRE |
| | YDYWGQGTLVTVSS |
| C04 | SEQ ID NO: 460 |
| con | X₁VQLVESGGGLVQX₂GGSLRLSCX₃ASGFTSPYSTIGWFRQAPGKX₄REGVACISS |
| | SGRGVNANYIDSVRGRFTISRDNX₅NTVYLQMNX₆LKX₇X₈DTAX₉YYCATNCWRLT |
| | SPAADNWGQGTX₁₀VTVSS |
| | X₁ is E or Q |
| | X₂ is P or S |
| | X₃ is S or V |
| | X₄ is E or G |
| | X₅ is A or S |
| | X₆ is K or S |
| | X₇ is A or P |
| | X₈ is D or E |
| | X₉ is I or V |
| | X₁₀ is M or Q |
| C04 | SEQ ID NO: 461 |
| | QVQLVESGGGLVQSGGSLRLSCVASGFTSPYSTIGWFRQAPGKEREGVACISSSGR |
| | GVNANYIDSVRGRFTISRDNAKNTVYLQMNKLKPDDTAIYYCATNCWRLTSPAADN |
| | WGQGTQVTVSS |
| huC04 | SEQ ID NO: 462 |
| | EVQLVESGGGLVQPGGSLRLSCSASGFTSPYSTIGWFRQAPGKGREGVACISSSGR |
| | GVNANYIDSVRGRFTISRDNSKNTVYLQMNSLRAEDTAVYYCATNCWRLTSPAADN |
| | WGQGTMVTVSS |
| C10 | SEQ ID NO: 463 |
| | DLQLVESGGGLVQPGGSLRLSCAASGLTLDYYSISWFRQGPGNEREAVSCISRSGG |
| | RINYADSVKGRFTISRDSAKNTVYLQMNSLKPEDTAVYYCAATRLPLSGLCVIGSS |
| | DNALWGQGTQVTVSS |
| huC10 | SEQ ID NO: 464 |
| | ELQLVESGGGLVQPGGSLRLSCAASGLTLDYYSISWFRQAPGKEREAVSCISRSGG |
| | RINYADSVKGRFTISRDSSKNTVYLQMNSLRAEDTAVYYCAATRLPLSGLCVIGSS |
| | DNALWGQGTLVTVSS |
| D01 | SEQ ID NO: 465 |
| | QVQLQESGGGLVQAGGSLRLSCAASGNFSSISTMAWYRQVPGKQREWVARIIWSSG |
| | ATNYADSVKGRFTASRDNTKNTMYLQMDRLTPEDTAVYLCAGAISQRSGTFWTYWG |
| | QGTQVTVSS |
| huD01 | SEQ ID NO: 466 |
| | QVQLLESGGGLVKPGGSLRLSCAASGNFSSISTMAWYRQAPGKQREWVARIIWSSG |
| | ATNYADSVKGRFTASRDNAKNSMYLQMNSLRAEDTAVYYCAGAISQRSGTFWTYWG |
| | QGTLVTVSS |
| E08 | SEQ ID NO: 467 |
| | QLQLVESGGGLVQPGGSLTLSCASSISTINGYAVAWFRQAPGKERSAVSSISSSGI |
| | RTNYKDSVKGRFTIWRDNAKAAVYLQLNNLEPEDTAMYYCAAKRGPILFGTYTHWG |
| | QGTQVTVSS |
| huE08 | SEQ ID NO: 468 |
| | QLQLVESGGGLVKPGGSLRLSCAASGSTLNGYAVAWFRQAPGKEREAVSSISSSGI |
| | RTNYKDSVKGRFTISRDNAKNSVYLQMNSLRAEDTAVYYCAAKRGPILFGTYTHWG |
| | QGTLVTVSS |
| E10 | SEQ ID NO: 469 |
| | EVQLVESGGGLVQAGGSLRLSCAASGSALSIFAMGWYRQTPGKQSVSVAAVTLRSG |
| | ITSYLDSVKGRFIISIDRAKNTMYLEMNDLKPEDTAIYYCNVRDYRSRDYWGQGTQ |
| | VTVSS |
| huE10 | SEQ ID NO: 470 |
| | EVQLVESGGGLVQPGGSLRLSCAASGSALSIFAMGWYRQAPGKQSESVAAVTLRSG |
| | ITSYLDSVKGRFTISIDRSKNTMYLQMNSLRAEDTAVYYCNVRDYRSRDYWGQGTL |
| | VTVSS |
| G01 | SEQ ID NO: 471 |
| | QVQLQESGGGLVQPGGSLRLSCAASGFTVDRYAIGWFRQAPGKEREGVQCITTSGG |
| | GSNYADSVKGRFTIARDNAKNTVYLQMDSLKPEDTAIYYCAATPLNYYCGIDFNPD |
| | KYDYWGQGTQVTVSS |
| huG01 | SEQ ID NO: 472 |
| | EVQLLESGGGLVQPGGSLRLSCAASGFTVDRYAIGWFRQAPGKEREGVQCITTSGG |
| | GSNYADSVKGRFTISRDNSKNTVYLQMNSLRAEDTAVYYCAATPLNYYCGIDFNPD |
| | KYDYWGQGTMVTVSS |
| G02 | SEQ ID NO: 473 |
| | QVQLVESGGGLVQPGGSLRLSCVASETIINFAPMGWYRQAPGTERELVAAINRDFS |
| | TYYANSVKGRFTISRDNAKKMLSLQMNSLNPEDTAMYYCAASPGRIWSGTILPPAP |
| | DRVVSWGQGTQVTVSS |
| huG02 | SEQ ID NO: 474 |
| | QVQLVESGGGLVQPGGSLRLSCSASGTIINFAPMGWYRQAPGKERELVAAINRDFS |
| | TYYANSVKGRFTISRDNSKNTLYLQMNSLRAEDTAVYYCAASPGRIWSGTILPPAP |
| | DRVVSWGQGTMVTVSS |

In some embodiments, the VHH binds to an epitope on IL-10Rβ comprising contact amino acid residues S78, S80, K81, Y82, V108, N138, E139, Y140, E141, T142, W143, N147, V148 and N150, wherein the amino acid residue positions are with respect to SEQ ID NO: 712 (e.g., VHH A05). In some embodiments, the VHH binds to an epitope on IL-10Rβ comprising contact amino acid residues N26, R28, N30, V32, Q39, E41, T72, D84, 1103, T104, D110, I112, P115, Q157, Q190, R192, F194, N199, A201, E203, W204 and E206, wherein the amino acid residue positions are with respect to SEQ ID NO: 712 (e.g., VHH C04).

Human IL-10Rβ amino acid sequence - NP_000619.3 /
Uniprot Q08334
(SEQ ID NO: 712)

| | |
|---|---|
| 1 | MAWSLGSWLG GCLLVSALGM VPPPENVRMN SVNFKNILQW |
| | ESPAFAKGNL TFTAQYLSYR |
| 61 | IFQDKCMNTT LTECDFSSLS KYGDHTLRVR AEFADEHSDW |
| | VNITFCPVDD TIIGPPGMQV |
| 121 | EVLADSLHMR FLAPKIENEY ETWTMKNVYN SWTYNVQYWK |
| | NGTDEKFQIT PQYDFEVLRN |
| 181 | LEPWTTYCVQ VRGFLPDRNK AGEWSEPVCE QTTHDETVPS |
| | WMVAVILMAS VEMVCLALLG |
| 241 | CFALLWCVYK KTKYAFSPRN SLPQHLKEFL GHPHHNTLLF |
| | FSFPLSDEND VFDKLSVIAE |
| 301 | DSESGKQNPG DSCSLGTPPG QGPQS |

### 4. IL-10Rα/IL-10Rβ Bispecific Binders

Provided are bispecific antibodies comprising a first immunoglobulin variable domain (VHH) and a second VHH, wherein the first VHH specifically binds to the extracellular domain of interleukin-10 receptor (IL-R) alpha subunit (IL-10Rα) and the second VHH specifically binds to the extracellular domain of interleukin-10 receptor beta subunit (IL-10Rβ). With respect to function, in some embodiments, the bispecific antibody is an IL-10R agonist that selectively suppresses activation of myeloid cells and induces minimal or no direct activation of T cells or B cells. In some embodiments, the bispecific antibody is an IL-10R agonist that predominantly suppresses activation of myeloid cells and induces minimal or no direct activation of T cells or B cells. In some embodiments, the bispecific antibody is an IL-10R agonist suppressing activation of myeloid cells (e.g., macrophages, monocytes and dendritic cells) with a half maximal effective concentration (EC50) less than about 10 pM and activating T cells and B cells below a predetermined threshold level at a concentration above about 1000 nM, e.g., above about 1500 nM, e.g., above about 2000 nM, e.g., above about 2500 nM, e.g., above about 3000 nM, e.g., above about 3500 nM, e.g., above about 4000 nM, e.g., above about 4500 nM, e.g., above about 5000 nM. In some embodiments, the bispecific antibody activates T cells or B cells with a maximum effect (Emax) that is 15% or less, e.g., 10% or less, e.g., 5% or less, in comparison to wild-type IL 10. In some embodiments, the activation of T cells is measured by at least one of Granzyme B secretion and interferon-γ (IFN-γ) production. In some embodiments, the bispecific antibody does not induce detectable granzyme B secretion from CD8+ T cells. In some embodiments, the bispecific antibody induces interferon-γ (IFN-γ) from CD8+ T cells less than 15%, e.g., less than 10%, e.g., less than 5%, the level of IFN-γ induced by wild-type IL-10. In some embodiments, the bispecific antibody agonizes the IL-10 receptor in a tyrosine kinase 2 (TYK2; NCBI Gene ID: 7297) dependent manner (i.e., requiring a functional TYK2, e.g., requiring the phosphorylation activity of TYK2). In some embodiments, the bispecific antibody induces minimal (e.g., less than 25% in comparison to wild-type IL-10) or no inhibition of IL 10Rβ dimerization with other IL-10 family cytokine receptor subunits, e.g., IL-10Rβ dimerization with IL-20Rα, IL-22R, IL-28R or IL-29R. In some embodiments, the bispecific antibody induces minimal (e.g., less than 25% in comparison to wild-type IL-10) or no inhibition of intracellular signaling of other IL-10Rβ-binding cytokines, e.g., minimal or no inhibition of IL-22, IL-26, IL-28A, IL-28B, or IL-29 intracellular signaling. In some embodiments, the second VHH binds to IL 10Rβ with an equilibrium dissociation constant (K_{D}) that is at least 5-fold greater, e.g., at least 7-fold greater, e.g., at least 10-fold greater, 15-fold greater, 20-fold greater, 25-fold greater, or more, than the K_{D} of the first VHH binding to IL 10Rα, e.g., wherein the binding affinity is determined by surface plasmon resonance (SPR). In some embodiments, the first VHH binds to IL 10Rα with affinity that is at least 5-fold greater, e.g., at least 7-fold greater, e.g., at least 10-fold, 15-fold, 20-fold, 25-fold greater, or more, than the binding affinity of the second VHH binding to IL 10Rβ, e.g., wherein the binding affinity is determined by surface plasmon resonance (SPR). In some embodiments, the first variable domain binds to IL 10Rα with an equilibrium dissociation constant (K_{D}) of lower than 2 nM and the second variable domain binds to IL 10Rβ with a K_{D} of greater than 10 nM, e.g., greater than 20 nM, e.g., greater than 30 nM, e.g., greater than 40 nM, e.g., greater than 50 nM, e.g., wherein the binding affinity is determined by surface plasmon resonance (SPR).

In some embodiments, the first VHH binds to an epitope on IL 10Rα comprising contact amino acid residues W33, E35, E37, H40, 142, H44, T46, L79, S80, T124, A170, R172, F178, F180, H182, Q204, K215, M217, W218, K220 and E221, wherein the amino acid residue positions are with respect to SEQ ID NO: 711 and the second VHH binds to an epitope on IL-10Rβ comprising contact amino acid residues S78, S80, K81, Y82, V108, N138, E139, Y140, E141, T142, W143, N147, V148 and N150, wherein the amino acid residue positions are with respect to SEQ ID NO: 712 (e.g., E07-A05). In some embodiments, the first VHH binds to an epitope on IL 10Rα comprising contact amino acid residues I169, I171, K173, F178, F180, T181, H182, K183, K186, F190, L192, L193, S195, E197, and F201, wherein the amino acid residue positions are with respect to SEQ ID NO: 711 and the second VHH binds to an epitope on IL-10Rβ comprising contact amino acid residues N26, R28, N30, V32, Q39, E41, T72, D84, I103, T104, D110, I112, P115, Q157, Q190, R192, F194, N199, A201, E203, W204, and E206, wherein the amino acid residue positions are with respect to SEQ ID NO: 712 (e.g., F11-C04).

With respect to structure, in some embodiments, the bispecific antibody comprises at least one polypeptide comprising the first VHH in tandem with the second VHH (e.g., in a single fusion protein). In some embodiments, the polypeptide comprises from N-terminus to C-terminus the first VHH that specifically binds to IL 10Rα and the second VHH that specifically binds to IL 10Rβ. In some embodiments, the polypeptide comprises from N-terminus to C-terminus the second VHH that specifically binds to IL 10Rβ and the first VHH that specifically binds to IL 10Rα. In some embodiments, the first VHH and the second VHH are connected or fused via a linker. In some embodiments, the linker is selected from the group consisting of: GS (SEQ ID NO: 713), GGS (SEQ ID NO: 714), GGGS (SEQ ID NO: 715), GGSGGS (SEQ ID NO: 716), GGGSGGGS (SEQ ID NO: 717) and GGSGGSGGS (SEQ ID NO: 718). In some embodiments, the linker comprises GGGS (SEQ ID NO: 715). The amino acid sequences of illustrative bispecific antibodies described herein are provided in Tables E and F, below.

### First and Second VHH

In some embodiments of the bispecific antibody, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to Kabat, respectively:
1) SEQ ID NOs: 22, 23, 24, 291, 292 and 293; (877)
2) SEQ ID NOs: 22, 25, 26, 291, 294 and 295; (647)
3) SEQ ID NOs: 22, 27, 24, 291, 297 and 298; (195)
4) SEQ ID NOs: 22, 23, 24, 288, 289 and 290; (048)
5) SEQ ID NOs: 22, 25, 26, 288, 289 and 290; (049)
6) SEQ ID NOs: 22, 27, 24, 288, 289 and 290; (050)
7) SEQ ID NOs: 22, 23, 24, 296, 308 and 309; (274)
8) SEQ ID NOs: 22, 25, 26, 296, 308 and 309; (771)
9) SEQ ID NOs: 22, 27, 24, 296, 308 and 309; (772)
10) SEQ ID NOs: 4, 28, 29, 288, 289 and 290; (054)
11) SEQ ID NOs: 4, 28, 29, 291, 294 and 295; (883)
12) SEQ ID NOs: 4, 28, 29, 291, 294 and 295; (784)
13) SEQ ID NOs: 4, 28, 29, 291, 297 and 298; (785)
14) SEQ ID NOs: 4, 5, 6, 288, 289 and 290; (046)
15) SEQ ID NOs: 4, 5, 6, 291, 294 and 295; (875)
16) SEQ ID NOs: 4, 5, 6, 291, 294 and 295; (776)
17) SEQ ID NOs: 4, 5, 6, 291, 297 and 298; (777)
18) SEQ ID NOs: 4, 5, 6, 296, 308 and 309; (272)
19) SEQ ID NOs: 4, 15, 16, 288, 289 and 290; (052)
20) SEQ ID NOs: 4, 15, 16, 296, 308 and 309; (278)
21) SEQ ID NOs: 4, 17, 18, 288, 289 and 290; (047)
22) SEQ ID NOs: 4, 17, 18, 291, 292 and 293; (876)
23) SEQ ID NOs: 4, 17, 18, 291, 294 and 295; (778)
24) SEQ ID NOs: 4, 17, 18, 291, 297 and 298; (779)
25) SEQ ID NOs: 45, 46, 47, 288, 289 and 290; (062)
26) SEQ ID NOs: 45, 46, 47, 291, 292 and 293; (891)
27) SEQ ID NOs: 45, 46, 47, 291, 294 and 295; (780)
28) SEQ ID NOs: 45, 46, 47, 291, 297 and 298; (781)
29) SEQ ID NOs: 45, 46, 47, 296, 308 and 309; (290)
30) SEQ ID NOs: 4, 48, 6, 291, 292 and 293; (892)
31) SEQ ID NOs: 4, 48, 6, 291, 294 and 295; (793)
32) SEQ ID NOs: 4, 48, 6, 291, 297 and 298; (794)
33) SEQ ID NOs: 54, 55, 24, 291, 292 and 293; (894)
34) SEQ ID NOs: 54, 55, 24, 291, 294 and 295; (795)
35) SEQ ID NOs: 54, 55, 24, 291, 297 and 298; (796)
36) SEQ ID NOs: 4, 30, 31, 291, 292 and 293; (884)
37) SEQ ID NOs: 4, 30, 31, 291, 294 and 295; (786)
38) SEQ ID NOs: 4, 30, 31, 291, 297 and 298; (787)
39) SEQ ID NOs: 4, 36, 29, 288, 289 and 290; (058)
40) SEQ ID NOs: 33, 34, 35, 296, 308 and 309; (284)
41) SEQ ID NOs: 33, 34, 35, 296, 308 and 309; (183)
42) SEQ ID NOs: 33, 34, 35, 296, 308 and 309; (184)
43) SEQ ID NOs: 19, 37, 38, 302, 303 and 304; (239)
44) SEQ ID NOs: 19, 37, 38, 296, 308 and 309; (286) or
45) SEQ ID NOs: 19, 51, 52, 302, 303 and 304; (247)

In some embodiments of the bispecific antibody, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to IMGT, respectively:
1) SEQ ID NOs: 77, 78, 79, 329, 330 and 331; (877)
2) SEQ ID NOs: 77, 80, 81, 329, 332 and 333; (647)
3) SEQ ID NOs: 77, 82, 79, 334, 335 and 336; (195)
4) SEQ ID NOs: 77, 78, 79, 326, 327 and 328; (048)
5) SEQ ID NOs: 77, 80, 81, 326, 327 and 328; (049)
6) SEQ ID NOs: 77, 82, 79, 326, 327 and 328; (050)
7) SEQ ID NOs: 77, 78, 79, 346, 347 and 348; (274)
8) SEQ ID NOs: 77, 80, 81, 346, 347 and 348; (771)
9) SEQ ID NOs: 77, 82, 79, 346, 347 and 348; (772)
10) SEQ ID NOs: 59, 83, 84, 326, 327 and 328; (054)
11) SEQ ID NOs: 59, 83, 84, 329, 330 and 331; (883)
12) SEQ ID NOs: 59, 83, 84, 329, 332 and 333; (784)
13) SEQ ID NOs: 59, 83, 84, 334, 335 and 336; (785)
14) SEQ ID NOs: 59, 60, 61, 326, 327 and 328; (046)
15) SEQ ID NOs: 59, 60, 61, 329, 330 and 331; (875)
16) SEQ ID NOs: 59, 60, 61, 329, 332 and 333; (776)
17) SEQ ID NOs: 59, 60, 61, 334, 335 and 336; (777)
18) SEQ ID NOs: 59, 60, 61, 346, 347 and 348; (272)
19) SEQ ID NOs: 59, 70, 71, 326, 327 and 328; (052)
20) SEQ ID NOs: 59, 70, 71, 346, 347 and 348; (278)
21) SEQ ID NOs: 59, 72, 73, 326, 327 and 328; (047)
22) SEQ ID NOs: 59, 72, 73, 329, 330 and 331; (876)
23) SEQ ID NOs: 59, 72, 73, 329, 332 and 333; (778)
24) SEQ ID NOs: 59, 72, 73, 334, 335 and 336; (779)
25) SEQ ID NOs: 98, 99, 100, 326, 327 and 328; (062)
26) SEQ ID NOs: 98, 99, 100, 329, 330 and 331; (891)
27) SEQ ID NOs: 98, 99, 100, 329, 332 and 333; (780)
28) SEQ ID NOs: 98, 99, 100, 334, 335 and 336; (781)
29) SEQ ID NOs: 98, 99, 100, 326, 327 and 328; (290)
30) SEQ ID NOs: 59, 101, 61, 329, 330 and 331; (892)
31) SEQ ID NOs: 59, 101, 61, 329, 332 and 333; (793)
32) SEQ ID NOs: 59, 101, 61, 334, 335 and 336; (794)
33) SEQ ID NOs: 106, 107, 79, 329, 330 and 331; (894)
34) SEQ ID NOs: 106, 107, 79, 329, 332 and 333; (795)
35) SEQ ID NOs: 106, 107, 79, 334, 335 and 336; (796)
36) SEQ ID NOs: 59, 85, 86, 329, 330 and 331; (884)
37) SEQ ID NOs: 59, 85, 86, 329, 332 and 333; (786)
38) SEQ ID NOs: 59, 85, 86, 334, 335 and 336; (787)
39) SEQ ID NOs: 59, 91, 84, 326, 327 and 328; (058)
40) SEQ ID NOs: 88, 89, 90, 326, 327 and 328; (284)
41) SEQ ID NOs: 88, 89, 90, 326, 327 and 328; (183)
42) SEQ ID NOs: 88, 89, 90, 326, 327 and 328; (184)
43) SEQ ID NOs: 92, 75, 93, 340, 341 and 342; (239)
44) SEQ ID NOs: 92, 75, 93, 326, 327 and 328; (286) or
45) SEQ ID NOs: 74, 75, 104, 340, 341 and 342; (247).

In some embodiments of the bispecific antibody, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to Chothia, respectively:
1) SEQ ID NOs: 127, 128, 129, 369, 370 and 371; (877)
2) SEQ ID NOs: 127, 130, 131, 369, 372 and 373; (647)
3) SEQ ID NOs: 127, 130, 131, 374, 375 and 376; (195)
4) SEQ ID NOs: 127, 128, 129, 366, 367 and 368; (048)
5) SEQ ID NOs: 127, 130, 131, 366, 367 and 368; (049)
6) SEQ ID NOs: 127, 130, 131, 366, 367 and 368; (050)
7) SEQ ID NOs: 127, 128, 129, 386, 387 and 388; (274)
8) SEQ ID NOs: 127, 130, 131, 386, 387 and 388; (771)
9) SEQ ID NOs: 127, 130, 131, 386, 387 and 388; (772)
10) SEQ ID NOs: 111, 132, 133, 366, 367 and 368; (054)
11) SEQ ID NOs: 111, 132, 133, 369, 370 and 371; (883)
12) SEQ ID NOs: 111, 132, 133, 369, 372 and 373; (784)
13) SEQ ID NOs: 111, 132, 133, 374, 375 and 376; (785)
14) SEQ ID NOs: 111, 112, 113, 366, 367 and 368; (046)
15) SEQ ID NOs: 111, 112, 113, 369, 370 and 371; (875)
16) SEQ ID NOs: 111, 112, 113, 369, 372 and 373; (776)
17) SEQ ID NOs: 111, 112, 113, 374, 375 and 376; (777)
18) SEQ ID NOs: 111, 112, 113, 386, 387 and 388; (272)
19) SEQ ID NOs: 111, 121, 122, 366, 367 and 368; (052)
20) SEQ ID NOs: 111, 121, 122, 386, 387 and 388; (278)
21) SEQ ID NOs: 111, 121, 123, 366, 367 and 368; (047)
22) SEQ ID NOs: 111, 121, 123, 369, 370 and 371; (876)
23) SEQ ID NOs: 111, 121, 123, 369, 372 and 373; (778)
24) SEQ ID NOs: 111, 121, 123, 374, 375 and 376; (779)
25) SEQ ID NOs: 145, 146, 147, 366, 367 and 368; (062)
26) SEQ ID NOs: 145, 146, 147, 369, 370 and 371; (891)
27) SEQ ID NOs: 145, 146, 147, 369, 372 and 373; (780)
28) SEQ ID NOs: 145, 146, 147, 374, 375 and 376; (781)
29) SEQ ID NOs: 145, 146, 147, 386, 387 and 388; (290)
30) SEQ ID NOs: 111, 112, 113, 369, 370 and 371; (892)
31) SEQ ID NOs: 111, 112, 113, 369, 372 and 373; (793)
32) SEQ ID NOs: 111, 112, 113, 374, 375 and 376; (794)
33) SEQ ID NOs: 152, 121, 129, 369, 370 and 371; (894)
34) SEQ ID NOs: 152, 121, 129, 369, 372 and 373; (795)
35) SEQ ID NOs: 152, 121, 129, 374, 375 and 376; (796)
36) SEQ ID NOs: 111, 134, 135, 369, 370 and 371; (884)
37) SEQ ID NOs: 111, 134, 135, 369, 372 and 373; (786)
38) SEQ ID NOs: 111, 134, 135, 374, 375 and 376; (787)
39) SEQ ID NOs: 111, 132, 133, 366, 367 and 368; (058)
40) SEQ ID NOs: 137, 138, 139, 386, 387 and 388; (284)
41) SEQ ID NOs: 137, 138, 139, 386, 387 and 388; (183)
42) SEQ ID NOs: 137, 138, 139, 386, 387 and 388; (184)
43) SEQ ID NOs: 140, 125, 141, 380, 381 and 381; (239)
44) SEQ ID NOs: 140, 125, 141, 386, 387 and 388; (286) or
45) SEQ ID NOs: 124, 125, 126, 380, 381 and 381 (247).

In some embodiments of the bispecific antibody, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR)
1, a VHH2-CDR2, and a VHH2-CDR3, comprising the following amino acid sequences, according to Honegger, respectively:
1) SEQ ID NOs: 174, 175, 176, 409, 410 and 411; (877)
2) SEQ ID NOs: 174, 177, 178, 409, 412 and 413; (647)
3) SEQ ID NOs: 174, 179, 176, 414, 415 and 416; (195)
4) SEQ ID NOs: 174, 175, 176, 406, 407 and 408; (048)
5) SEQ ID NOs: 174, 177, 178, 406, 407 and 408; (049)
6) SEQ ID NOs: 174, 179, 176, 406, 407 and 408; (050)
7) SEQ ID NOs: 174, 175, 176, 426, 427 and 428; (274)
8) SEQ ID NOs: 174, 177, 178, 426, 427 and 428; (771)
9) SEQ ID NOs: 174, 179, 176, 426, 427 and 428; (772)
10) SEQ ID NOs: 156, 180, 181, 406, 407 and 408; (054)
11) SEQ ID NOs: 156, 180, 181, 409, 410 and 411; (883)
12) SEQ ID NOs: 156, 180, 181, 409, 412 and 413; (784)
13) SEQ ID NOs: 156, 180, 181, 414, 415 and 416; (785)
14) SEQ ID NOs: 156, 157, 158, 406, 407 and 408; (046)
15) SEQ ID NOs: 156, 157, 158, 409, 410 and 411; (875)
16) SEQ ID NOs: 156, 157, 158, 409, 412 and 413; (776)
17) SEQ ID NOs: 156, 157, 158, 414, 415 and 416; (777)
18) SEQ ID NOs: 156, 157, 158, 426, 427 and 428; (272)
19) SEQ ID NOs: 156, 167, 168, 406, 407 and 408; (052)
20) SEQ ID NOs: 156, 167, 168, 426, 427 and 428; (278)
21) SEQ ID NOs: 156, 169, 170, 406, 407 and 408; (047)
22) SEQ ID NOs: 156, 169, 170, 409, 410 and 411; (876)
23) SEQ ID NOs: 156, 169, 170, 409, 412 and 413; (778)
24) SEQ ID NOs: 156, 169, 170, 414, 415 and 416; (779)
25) SEQ ID NOs: 198, 199, 200, 406, 407 and 408; (062)
26) SEQ ID NOs: 198, 199, 200, 409, 410 and 411; (891)
27) SEQ ID NOs: 198, 199, 200, 409, 412 and 413; (780)
28) SEQ ID NOs: 198, 199, 200, 414, 415 and 416; (781)
29) SEQ ID NOs: 198, 199, 200, 426, 427 and 428; (290)
30) SEQ ID NOs: 156, 201, 158, 409, 410 and 411; (892)
31) SEQ ID NOs: 156, 201, 158, 409, 412 and 413; (793)
32) SEQ ID NOs: 156, 201, 158, 414, 415 and 416; (794)
33) SEQ ID NOs: 208, 209, 176, 409, 410 and 411; (894)
34) SEQ ID NOs: 208, 209, 176, 409, 412 and 413; (795)
35) SEQ ID NOs: 208, 209, 176, 414, 415 and 416; (796)
36) SEQ ID NOs: 156, 182, 183, 409, 410 and 411; (884)
37) SEQ ID NOs: 156, 182, 183, 409, 412 and 413; (786)
38) SEQ ID NOs: 156, 182, 183, 414, 415 and 416; (787)
39) SEQ ID NOs: 156, 188, 181, 406, 407 and 408; (058)
40) SEQ ID NOs: 185, 186, 187, 426, 427 and 428; (284)
41) SEQ ID NOs: 709, 186, 187, 426, 427 and 428; (183)
42) SEQ ID NOs: 709, 186, 187, 426, 427 and 428; (184)
43) SEQ ID NOs: 189, 190, 191, 420, 421 and 422; (239)
44) SEQ ID NOs: 189, 190, 191, 426, 427 and 428; (286) or
45) SEQ ID NOs: 204, 205, 206, 420, 421 and 422; (247).

In some embodiments of the bispecific antibody, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 212 and 448; [A06con-A05con]
2) SEQ ID NOs: 212 and 449; [A06con-A05con]
3) SEQ ID NOs: 212 and 460; [A06con-C04con]
4) SEQ ID NOs: 233 and 448; [E07con-A05con]
5) SEQ ID NOs: 234 and 449; [E07con-A05con]
6) SEQ ID NOs: 233 and 460; [E07con-C04con]
7) SEQ ID NOs: 234 and 460; [E07con-C04con]
8) SEQ ID NOs: 710 and 448; [C09con-A05con]
9) SEQ ID NOs: 710 and 449; [C09con-A05con]10) SEQ ID NOs: 710 and 460; [C09con-C04con]11) SEQ ID NOs: 230 and 460 [G22con-C04con] or 12) SEQ ID NOs: 249 and 460 [F11con-C04con].

In some embodiments of the bispecific antibody, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 235 and 450; (877)
2) SEQ ID NOs: 237 and 451; (647)
3) SEQ ID NOs: 239 and 452; (195)
4) SEQ ID NOs: 235 and 446; (048)
5) SEQ ID NOs: 237 and 447; (049)
6) SEQ ID NOs: 239 and 447; (050)
7) SEQ ID NOs: 235 and 461; (274)
8) SEQ ID NOs: 237 and 462; (771)
9) SEQ ID NOs: 239 and 462; (772)
10) SEQ ID NOs: 241 and 446; (054)
11) SEQ ID NOs: 243 and 447; (755)
12) SEQ ID NOs: 241 and 450; (883)
13) SEQ ID NOs: 243 and 451; (784)
14) SEQ ID NOs: 243 and 452; (785)
15) SEQ ID NOs: 213 and 446; (046)
16) SEQ ID NOs: 215 and 447; (747)
17) SEQ ID NOs: 213 and 450; (875)
18) SEQ ID NOs: 215 and 451; (776)
19) SEQ ID NOs: 215 and 452; (777)
20) SEQ ID NOs: 213 and 461; (272)
21) SEQ ID NOs: 215 and 462; (773)
22) SEQ ID NOs: 224 and 446; (052)
23) SEQ ID NOs: 226 and 447; (753)
24) SEQ ID NOs: 224 and 461; (278)
25) SEQ ID NOs: 226 and 462; (769)
26) SEQ ID NOs: 227 and 446; (047)
27) SEQ ID NOs: 229 and 447; (748)
28) SEQ ID NOs: 227 and 450; (876)
29) SEQ ID NOs: 229 and 451; (778)
30) SEQ ID NOs: 229 and 452; (779)
31) SEQ ID NOs: 267 and 446; (062)
32) SEQ ID NOs: 269 and 447; (763)
33) SEQ ID NOs: 267 and 450; (891)
34) SEQ ID NOs: 269 and 451; (780)
35) SEQ ID NOs: 269 and 452; (781)
36) SEQ ID NOs: 267 and 461; (290)
37) SEQ ID NOs: 269 and 462; (791)
38) SEQ ID NOs: 270 and 450; (892)
39) SEQ ID NOs: 272 and 451; (793)
40) SEQ ID NOs: 272 and 452; (794)
41) SEQ ID NOs: 285 and 450; (894)
42) SEQ ID NOs: 287 and 451; (795)
43) SEQ ID NOs: 287 and 452; (796)
44) SEQ ID NOs: 244 and 450; (884)
45) SEQ ID NOs: 246 and 451; (786)
46) SEQ ID NOs: 247 and 452; (787)
47) SEQ ID NOs: 253 and 446; (058)
48) SEQ ID NOs: 255 and 447; (759)
49) SEQ ID NOs: 250 and 461; (284)
50) SEQ ID NOs: 251 and 462; (183)
51) SEQ ID NOs: 252 and 462; (184)
52) SEQ ID NOs: 256 and 456; (239)
53) SEQ ID NOs: 258 and 457; (740)
54) SEQ ID NOs: 256 and 461; (286)
55) SEQ ID NOs: 258 and 462; (788)
56) SEQ ID NOs: 278 and 456; (247) or
57) SEQ ID NOs: 280 and 457; (749).

In some embodiments of the bispecific antibody, the first VHH that specifically binds to IL 10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 236 and 450; (877)
2) SEQ ID NOs: 238 and 451; (647)
3) SEQ ID NOs: 240 and 452; (195)
4) SEQ ID NOs: 236 and 446; (048)
5) SEQ ID NOs: 238 and 447; (049)
6) SEQ ID NOs: 240 and 447; (050)
7) SEQ ID NOs: 236 and 461; (274)
8) SEQ ID NOs: 238 and 462; (771)
9) SEQ ID NOs: 240 and 462; (772)
10) SEQ ID NOs: 242 and 446; (054)
11) SEQ ID NOs: 243 and 447; (755)
12) SEQ ID NOs: 242 and 450; (883)
13) SEQ ID NOs: 243 and 451; (784)
14) SEQ ID NOs: 243 and 452; (785)
15) SEQ ID NOs: 214 and 446; (046)
16) SEQ ID NOs: 215 and 447; (747)
17) SEQ ID NOs: 214 and 450; (875)
18) SEQ ID NOs: 215 and 451; (776)
19) SEQ ID NOs: 215 and 452; (777)
20) SEQ ID NOs: 214 and 461; (272)
21) SEQ ID NOs: 215 and 462; (773)
22) SEQ ID NOs: 225 and 446; (052)
23) SEQ ID NOs: 226 and 447; (753)
24) SEQ ID NOs: 225 and 461; (278)
25) SEQ ID NOs: 226 and 462; (769)
26) SEQ ID NOs: 228 and 446; (047)
27) SEQ ID NOs: 229 and 447; (748)
28) SEQ ID NOs: 228 and 450; (876)
29) SEQ ID NOs: 229 and 451; (778)
30) SEQ ID NOs: 229 and 452; (779)
31) SEQ ID NOs: 268 and 446; (062)
32) SEQ ID NOs: 269 and 447; (763)
33) SEQ ID NOs: 268 and 450; (891)
34) SEQ ID NOs: 269 and 451; (780)
35) SEQ ID NOs: 269 and 452; (781)
36) SEQ ID NOs: 268 and 461; (290)
37) SEQ ID NOs: 269 and 462; (791)
38) SEQ ID NOs: 271 and 450; (892)
39) SEQ ID NOs: 273 and 451; (793)
40) SEQ ID NOs: 274 and 452; (794)
41) SEQ ID NOs: 286 and 450; (894)
42) SEQ ID NOs: 287 and 451; (795)
43) SEQ ID NOs: 287 and 452; (796)
44) SEQ ID NOs: 245 and 450; (884)
45) SEQ ID NOs: 246 and 451; (786)
46) SEQ ID NOs: 246 and 452; (787)
47) SEQ ID NOs: 254 and 446; (058)
48) SEQ ID NOs: 255 and 447; (759)
49) SEQ ID NOs: 250 and 461; (284)
50) SEQ ID NOs: 251 and 462; (183)
51) SEQ ID NOs: 252 and 462; (184)
52) SEQ ID NOs: 257 and 456; (239)
53) SEQ ID NOs: 259 and 457; (740)
54) SEQ ID NOs: 257 and 461; (286)
55) SEQ ID NOs: 259 and 462; (788)
56) SEQ ID NOs: 279 and 456; (247) or
57) SEQ ID NOs: 280 and 457; (749).

In some embodiments, the bispecific antibody comprises a polypeptide comprising in tandem the first VHH that specifically binds to IL 10Rα and the second VHH that specifically binds to IL-10Rβ, comprising the following amino acid sequence, or an amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 475-476 and 573.

In some embodiments, the bispecific antibody comprises a polypeptide comprising in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 574, 576, 578, 580, 582, 584, 586, 588 and 590.

In some embodiments, the bispecific antibody comprises a polypeptide comprising in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 575, 577, 579, 581, 583, 585, 587, 589, 591, 737-751 and 757-758.

In some embodiments, the first VHH that specifically binds to IL 10Rα and the second VHH that specifically binds to IL-10Rβ are humanized. Illustrative humanized VHH sequences provided herein are designated with an “hu” prefix.

Amino acid sequence of illustrative αIL-10Rα/αIL-10Rβ Tandem Single Immunoglobulin Domain (VHH-VHH) are provided in Table E, below.

TABLE E
Illustrative αIL-10Rα / αIL-10Rβ Tandem Single
Immunoglobulin Domain (VHH-VHH) amino acid sequences
αIL-10Rα-αIL-10Rβ Tandem Single Immunoglobulin Domain (VHH-VHH)

| VHH-VHH | |
|---|---|
| name | VHH-VHH amino acid sequence |
| 774 | SEQ ID NO: 475 |
| E07-A05 | X₁X₂LQLVESGGGLVX₃X₄GX₅SLRLSCAASGRTFSAYGMGWFRQAPGKX₆REFVX₇ |
| con1 | AIRWX₈GX₉X₁₀TYYAX₁₁SVKGRFTISKDX₁₂X₁₃KNTLYLQMX₁₄SLX₁₅X₁₆EDTAV |
| | YYCAAKTX₁₇X₁₈X₁₉SYLSEKSYX₂₀YWGQGTX₂₁VTVSSX₂₂X₂₃VQLX₂₄ESGGGLV |
| | QPGGSLX₂₅LSCAASGX₂₆SLDX₂₇X₂₈X₂₉IGWFRX₃₀APGKX₃₁X₃₂EGVSX₃₃ISA |
| | X₃₄DX₃₅STHYAX₃₆X₃₇VKGRFTISRDNX₃₈KNTVX₃₉LQMNX₄₀LX₄₁X₄₂EDX₄₃X₄₄ |
| | X₄₅YYCAASLX₄₆GLGX₄₇KX₄₈EX₄₉DYDYWGQGTX₅₀VTVSS |
| | X₁ is S, GG, AS, ASS or absent (i.e., no amino acid |
| | residues) |
| | X₂ is E or Q |
| | X₃ is Q or R |
| | X₄ is A or P |
| | X₅ is D or G |
| | X₆ is E or G |
| | X₇ is A or S |
| | X₈ is I or Y |
| | X₉ is D or E |
| | X₁₀ is A, G or S |
| | X₁₁ is D or E |
| | X₁₂ is N or S |
| | X₁₃ is A or S |
| | X₁₄ is N or S |
| | X₁₅ is K or R |
| | X₁₆ is A or P |
| | X₁₇ is R or S |
| | X₁₈ is P or V |
| | X₁₉ is D or T |
| | X₂₀ is G or N |
| | X₂₁ is M or Q |
| | X₂₂ is GGGS, GGGGS, or GGGSGGGS |
| | X₂₃ is E or Q |
| | X₂₄ is L or Q |
| | X₂₅ is K or R |
| | X₂₆ is I or N |
| | X₂₇ is E or Y |
| | X₂₈ is G, P or S |
| | X₂₉ is V or T |
| | X₃₀ is Q or R |
| | X₃₁ is E or G |
| | X₃₂ is L or R |
| | X₃₃ is A or C |
| | X₃₄ is R or S |
| | X₃₅ is A or G |
| | X₃₆ is D or N |
| | X₃₇ is A or S |
| | X₃₈ is S or T |
| | X₃₉ is D or Y |
| | X₄₀ is N or S |
| | X₄₁ is K or R |
| | X₄₂ is A or P |
| | X₄₃ is A or T |
| | X₄₄ is A or G |
| | X₄₅ is H or V |
| | X₄₆ is H or W |
| | X₄₇ is A or C |
| | X₄₈ is R or S |
| | X₄₉ is A or E |
| | X₅₀ is L, M or Q |
| 775 | SEQ ID NO: 476 |
| E07-A05 | X₁QLQLVESGGGLVX₃X₄GGSLRLSCAASGRTFSAYGMGWFRQAPGKX₆REFVX₇A |
| con2 | IRWX₈GX₉X₁₀TYYAX₁₁SVKGRFTISKDNX₁₃KNTLYLQMX₁₄SLX₁₅X₁₆EDTAVYY |
| | CAAKTX₁₇X₁₈X₁₉SYLSEKSYX₂₀YWGQGTX₂₁VTVSSX₂₂X₂₃VQLVESGGGLVQPG |
| | GSLRLSCAASGNSLDYPTIGWFRQAPGKX₂₄X₂₅EGVSX₂₆ISASDX₂₇STHYADX₂₈ |
| | VKGRFTISRDNX₂₉KNTVX₃₀LQMNX₃₁LX₃₂X₃₃EDTX₃₄X₃₅YYCAASLX₃₆GLGX₃₇ |
| | KSEADYDYWGQGTX₃₈VTVSS |
| | X₁ is S, GG, AS, ASS or absent (i.e., no amino acid |
| | residues) |
| | X₃ is Q or R |
| | X₄ is A or P |
| | X₆ is E or G |
| | X₇ is A or S |
| | X₈ is I or Y |
| | X₉ is D or E |
| | X₁₀ is A, G or S |
| | X₁₁ is D or E |
| | X₁₃ is A or S |
| | X₁₄ is N or S |
| | X₁₅ is K or R |
| | X₁₆ is A or P |
| | X₁₇ is R or S |
| | X₁₈ is P or V |
| | X₁₉ is D or T |
| | X₂₀ is G or N |
| | X₂₁ is M or Q |
| | X₂₂ is GGGS, GGGGS, or GGGSGGGS |
| | X₂₃ is E or Q |
| | X₂₄ is E or G |
| | X₂₅ is L or R |
| | X₂₆ is A or C |
| | X₂₇ is A or G |
| | X₂₈ is A or S |
| | X₂₉ is S or T |
| | X₃₀ is D or Y |
| | X₃₁ is N or S |
| | X₃₂ is K or R |
| | X₃₃ is A or P |
| | X₃₄ is A or G |
| | X₃₅ is H or V |
| | X₃₆ is H or W |
| | X₃₇ is A or C |
| | X₃₈ is M or Q |
| 877 | SEQ ID NO: 477 |
| E07-A05 | X₁ is S, GG, AS, ASS or absent (i.e., no amino acid |
| | residues) |
| | X₂ is GGGS, GGGGS, or GGGSGGGS |
| | SEQ ID NO: 478 |
| | X₁ is AS; X₂ is GGGS |
| | X₁QLQLVESGGGLVRAGGSLRLSCAASGRTFSAYGMGWFRQAPGKEREFVAAIRW |
| | IGDSTYYADSVKGRFTISKDNAKNTLYLQMSSLKPEDTAVYYCAAKTSVTSYLS |
| | EKSYNYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSS |
| 647 | SEQ ID NO: 479 |
| huE07- | X₁ is S, GG, AS, ASS or absent (i.e., no amino acid |
| huA05 | residues) |
| | X₂ is GGGS, GGGGS, or GGGSGGGS |
| | SEQ ID NO: 480 |
| | X₁ is AS; X₂ is GGGS |
| | X₁QLQLLESGGGLVQPGGSLRLSCAASGRTFSAYGMGWFRQAPGKGREFVSAIRW |
| | YGEGTYYAESVKGRFTISKDNSKNTLYLQMNSLRAEDTAVYYCAAKTRPDSYLS |
| | EKSYGYWGQGTMVTVSSX₂EVQLVESGGGLVQPGGSLRLSCAASGNSLDYPTIGW |
| | FRQAPGKGLEGVSAISASDASTHYADAVKGRFTISRDNSKNTVYLQMNSLRAED |
| | TAVYYCAASLHGLGAKSEADYDYWGQGTMVTVSS |
| 195 | SEQ ID NO: 481 |
| huE07- | X₁ is S, GG, AS, ASS or absent (i.e., no amino acid |
| huA05 | residues) |
| | X₂ is GGGS, GGGGS, or GGGSGGGS |
| | SEQ ID NO: 482 |
| | X₁ is AS; X₂ is GGGS |
| | X₁QLQLLESGGGLVQPGGSLRLSCAASGRTFSAYGMGWFRQAPGKGREFVSAIRW |
| | IGDATYYAESVKGRFTISKDNSKNTLYLQMNSLRAEDTAVYYCAAKTSVTSYLS |
| | EKSYNYWGQGTMVTVSSX₂EVQLVESGGGLVQPGGSLRLSCAASGNSLDYSTIGW |
| | FRQAPGKGLEGVSCISASEGSTHYADAVKGRFTISRDNSKNTVYLQMNSLRAED |
| | TAVYYCAASLHGLGCKSEADYDYWGQGTMVTVSS |
| 048 | SEQ ID NO: 483 |
| E07-A02 | X₁ is S, GG, AS, ASS or absent (i.e., no amino acid |
| | residues) |
| | X₂ is GGGS, GGGGS, or GGGSGGGS |
| | SEQ ID NO: 484 |
| | X₁ is AS; X₂ is GGGS |
| | X₁QLQLVESGGGLVRAGGSLRLSCAASGRTFSAYGMGWFRQAPGKEREFVAAIRW |
| | IGDSTYYADSVKGRFTISKDNAKNTLYLQMSSLKPEDTAVYYCAAKTSVTSYLS |
| | EKSYNYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSS |
| 049 | SEQ ID NO: 485 |
| huE07- | X₁ is S, GG, AS, ASS or absent (i.e., no amino acid |
| huA02 | residues) |
| | X₂ is GGGS, GGGGS, or GGGSGGGS |
| | SEQ ID NO: 486 |
| | X₁ is AS; X₂ is GGGS |
| | X₁QLQLLESGGGLVQPGGSLRLSCAASGRTFSAYGMGWFRQAPGKGREFVSAIRW |
| | YGEGTYYAESVKGRFTISKDNSKNTLYLQMNSLRAEDTAVYYCAAKTRPDSYLS |
| | EKSYGYWGQGTMVTVSSX₂EVQLLESGGGLVQPGGSLRLSCAASGISLDEGVIGW |
| | FRQAPGKEREGVSCISARDGSTHYANAVKGRFTISRDNSKNTVYLQMNSLRAED |
| | TAVYYCAASLWGLGCKREEDYDYWGQGTLVTVSS |
| 050 | SEQ ID NO: 487 |
| huE07- | X₁ is S, GG, AS, ASS or absent (i.e., no amino acid |
| huA02 | residues) |
| | X₂ is GGGS, GGGGS, or GGGSGGGS |
| | SEQ ID NO: 488 |
| | X₁ is AS; X₂ is GGGS |
| | X₁QLQLLESGGGLVQPGGSLRLSCAASGRTFSAYGMGWFRQAPGKGREFVSAIRW |
| | IGDATYYAESVKGRFTISKDNSKNTLYLQMNSLRAEDTAVYYCAAKTSVTSYLS |
| | EKSYNYWGQGTMVTVSSX₂EVQLLESGGGLVQPGGSLRLSCAASGISLDEGVIGW |
| | FRQAPGKEREGVSCISARDGSTHYANAVKGRFTISRDNSKNTVYLQMNSLRAED |
| | TAVYYCAASLWGLGCKREEDYDYWGQGTLVTVSS |
| 274 | SEQ ID NO: 489 |
| E07-C04 | X₁ is S, GG, AS, ASS or absent (i.e., no amino acid |
| | residues) |
| | X₂ is GGGS, GGGGS, or GGGSGGGS |
| | SEQ ID NO: 490 |
| | X₁ is AS; X₂ is GGGS |
| | X₁QLQLVESGGGLVRAGGSLRLSCAASGRTFSAYGMGWFRQAPGKEREFVAAIRW |
| | IGDSTYYADSVKGRFTISKDNAKNTLYLQMSSLKPEDTAVYYCAAKTSVTSYLS |
| | EKSYNYWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSS |
| 771 | SEQ ID NO: 491 |
| huE07- | X₁ is S, GG, AS, ASS or absent (i.e., no amino acid |
| huC04 | residues) |
| | X₂ is GGGS, GGGGS, or GGGSGGGS |
| | SEQ ID NO: 492 |
| | X₁ is AS; X₂ is GGGS |
| | X₁QLQLLESGGGLVQPGGSLRLSCAASGRTFSAYGMGWFRQAPGKGREFVSAIRW |
| | YGEGTYYAESVKGRFTISKDNSKNTLYLQMNSLRAEDTAVYYCAAKTRPDSYLS |
| | EKSYGYWGQGTMVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSS |
| 772 | SEQ ID NO: 493 |
| huE07- | X₁ is S, GG, AS, ASS or absent (i.e., no amino acid |
| huC04 | residues) |
| | X₂ is GGGS, GGGGS, or GGGSGGGS |
| | SEQ ID NO: 494 |
| | X₁ is AS; X₂ is GGGS |
| | X₁QLQLLESGGGLVQPGGSLRLSCAASGRTFSAYGMGWFRQAPGKGREFVSAIRW |
| | IGDATYYAESVKGRFTISKDNSKNTLYLQMNSLRAEDTAVYYCAAKTSVTSYLS |
| | EKSYNYWGQGTMVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSS |
| 054 | SEQ ID NO: 495 |
| E15-A02 | X₁ is S, GG, AS, ASS or absent (i.e., no amino acid |
| | residues) |
| | X₂ is GGGS, GGGGS, or GGGSGGGS |
| | SEQ ID NO: 496 |
| | X₁ is AS; X₂ is GGGS |
| | X₁QVQLQESGGGLVQAGGSLRLSCAASGRTFSSYGMGWFRQAPGKEREFVAAISW |
| | IGNSAYYADSVKGRFTLSGDNAKNTLYLQMNSLKPEDTAVYYCAAKYYTDSYYA |
| | AASYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSS |
| 755 | SEQ ID NO: 497 |
| huE15- | X₂ is GGGS, GGGGS, or GGGSGGGS |
| huA02 | SEQ ID NO: 498 |
| | X₂ is GGGS |
| | EVQLLESGGGLVQPGGSLRLSCAASGRTFSSYGMGWFRQAPGKEREFVAAISWI |
| | GNSAYYADSVKGRFTLSGDNSKNTLYLQMNSLRAEDTAVYYCAAKYYTDSYYAA |
| | ASYAYWGQGTLVTVSSX₂EVQLLESGGGLVQPGGSLRLSCAASGISLDEGVIGWF |
| | RQAPGKEREGVSCISARDGSTHYANAVKGRFTISRDNSKNTVYLQMNSLRAEDT |
| | AVYYCAASLWGLGCKREEDYDYWGQGTLVTVSS |
| 883 | SEQ ID NO: 499 |
| E15-A05 | X₁ is S, GG, AS, ASS or absent (i.e., no amino acid |
| | residues) |
| | X₂ is GGGS, GGGGS, or GGGSGGGS |
| | SEQ ID NO: 500 |
| | X₁ is AS; X₂ is GGGS |
| | X₁QVQLQESGGGLVQAGGSLRLSCAASGRTFSSYGMGWFRQAPGKEREFVAAISW |
| | IGNSAYYADSVKGRFTLSGDNAKNTLYLQMNSLKPEDTAVYYCAAKYYTDSYYA |
| | AASYAYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSS |
| 784 | SEQ ID NO: 501 |
| huE15- | X₂ is GGGS, GGGGS, or GGGSGGGS |
| huA05 | SEQ ID NO: 502 |
| | X₂ is GGGS |
| | EVQLLESGGGLVQPGGSLRLSCAASGRTFSSYGMGWFRQAPGKEREFVAAISWI |
| | GNSAYYADSVKGRFTLSGDNSKNTLYLQMNSLRAEDTAVYYCAAKYYTDSYYAA |
| | ASYAYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSS |
| 785 | SEQ ID NO: 503 |
| huE15- | X₂ is GGGS, GGGGS, or GGGSGGGS |
| huA05 | SEQ ID NO: 504 |
| | X₂ is GGGS |
| | EVQLLESGGGLVQPGGSLRLSCAASGRTFSSYGMGWFRQAPGKEREFVAAISWI |
| | GNSAYYADSVKGRFTLSGDNSKNTLYLQMNSLRAEDTAVYYCAAKYYTDSYYAA |
| | ASYAYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSS |
| 046 | SEQ ID NO: 505 |
| A06-A02 | X₁ is S, GG, AS, ASS or absent (i.e., no amino acid |
| | residues) |
| | X₂ is GGGS, GGGGS, or GGGSGGGS |
| | SEQ ID NO: 506 |
| | X₁ is AS; X₂ is GGGS |
| | X₁QLQLVESGGGLVQAGGSLRLSCAASGRTFSSYGMGWFRQAPGKERGLVAAINW |
| | IPGTTYYADSVKGRFTISGDKDKNTLYLQMDNLKPEDTAVYYCAARRRIDNYYR |
| | ASEYDYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSS |
| 747 | SEQ ID NO: 507 |
| huA06- | X₂ is GGGS, GGGGS, or GGGSGGGS |
| huA02 | SEQ ID NO: 508 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLLESGGGLVQPGGSLRLSCAASGISLDEGVIGWF |
| | RQAPGKEREGVSCISARDGSTHYANAVKGRFTISRDNSKNTVYLQMNSLRAEDT |
| | AVYYCAASLWGLGCKREEDYDYWGQGTLVTVSS |
| 875 | SEQ ID NO: 509 |
| A06-A05 | X₁ is S, GG, AS, ASS or absent (i.e., no amino acid |
| | residues) |
| | X₂ is GGGS, GGGGS, or GGGSGGGS |
| | SEQ ID NO: 510 |
| | X₁ is AS; X₂ is GGGS |
| | X₁QLQLVESGGGLVQAGGSLRLSCAASGRTFSSYGMGWFRQAPGKERGLVAAINW |
| | IPGTTYYADSVKGRFTISGDKDKNTLYLQMDNLKPEDTAVYYCAARRRIDNYYR |
| | ASEYDYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSS |
| 776 | SEQ ID NO: 511 |
| huA06- | X₂ is GGGS, GGGGS, or GGGSGGGS |
| huA05 | SEQ ID NO: 512 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSS |
| 777 | SEQ ID NO: 513 |
| huA06- | X₂ is GGGS, GGGGS, or GGGSGGGS |
| huA05 | SEQ ID NO: 514 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSS |
| 272 | SEQ ID NO: 515 |
| A06-C04 | X₁ is S, GG, AS, ASS or absent (i.e., no amino acid |
| | residues) |
| | X₂ is GGGS, GGGGS, or GGGSGGGS |
| | SEQ ID NO: 516 |
| | X₁ is AS; X₂ is GGGS |
| | X₁QLQLVESGGGLVQAGGSLRLSCAASGRTFSSYGMGWFRQAPGKERGLVAAINW |
| | IPGTTYYADSVKGRFTISGDKDKNTLYLQMDNLKPEDTAVYYCAARRRIDNYYR |
| | ASEYDYWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSS |
| 773 | SEQ ID NO: 517 |
| huA06- | X₂ is GGGS, GGGGS, or GGGSGGGS |
| huC04 | SEQ ID NO: 518 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLVESGGGLVQPGGSLRLSCSASGFTSPYSTIGWF |
| | RQAPGKGREGVACISSSGRGVNANYIDSVRGRFTISRDNSKNTVYLQMNSLRAE |
| | DTAVYYCATNCWRLTSPAADNWGQGTMVTVSS |
| 052 | SEQ ID NO: 519 |
| C09-A02 | X₁ is S, GG, AS, ASS or absent (i.e., no amino acid |
| | residues) |
| | X₂ is GGGS, GGGGS, or GGGSGGGS |
| | SEQ ID NO: 520 |
| | X₁ is AS; X₂ is GGGS |
| | X₁QVQLQESGGGLVQAGGSLRLSCAASGRTFSSYGMGWFRQAPGKEREFVAAISW |
| | IGGGTYYADSVKGRFTLSRDNAKNMVYLQMNSLKPEDTAVYYCAAKYYPDSYFA |
| | AASYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSS |
| 753 | SEQ ID NO: 521 |
| huC09- | X₂ is GGGS, GGGGS, or GGGSGGGS |
| huA02 | SEQ ID NO: 522 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLLESGGGLVQPGGSLRLSCAASGISLDEGVIGWF |
| | RQAPGKEREGVSCISARDGSTHYANAVKGRFTISRDNSKNTVYLQMNSLRAEDT |
| | AVYYCAASLWGLGCKREEDYDYWGQGTLVTVSS |
| 278 | SEQ ID NO: 523 |
| C09-C04 | X₁ is S, GG, AS, ASS or absent (i.e., no amino acid |
| | residues) |
| | X₂ is GGGS, GGGGS, or GGGSGGGS |
| | SEQ ID NO: 524 |
| | X₁ is AS; X₂ is GGGS |
| | X₁QVQLQESGGGLVQAGGSLRLSCAASGRTFSSYGMGWFRQAPGKEREFVAAISW |
| | IGGGTYYADSVKGRFTLSRDNAKNMVYLQMNSLKPEDTAVYYCAAKYYPDSYFA |
| | AASYAYWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSS |
| 769 | SEQ ID NO: 525 |
| huC09- | X₂ is GGGS, GGGGS, or GGGSGGGS |
| huC04 | SEQ ID NO: 526 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLVESGGGLVQPGGSLRLSCSASGFTSPYSTIGWF |
| | RQAPGKGREGVACISSSGRGVNANYIDSVRGRFTISRDNSKNTVYLQMNSLRAE |
| | DTAVYYCATNCWRLTSPAADNWGQGTMVTVSS |
| 047 | SEQ ID NO: 527 |
| D08-A02 | X₁ is S, GG, AS, ASS or absent (i.e., no amino acid |
| | residues) |
| | X₂ is GGGS, GGGGS, or GGGSGGGS |
| | SEQ ID NO: 528 |
| | X₁ is AS; X₂ is GGGS |
| | X₁QVQLQESGGGLVQAGGSLRLSCAASGRTFSSYGMGWFRQAPGKEREFVAAISW |
| | IGGSTYYADSVKGRFTLSSDNAKNRIALQMNSLKPEDTAVYYCAAKYYTDSYFA |
| | ATSYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSS |
| 748 | SEQ ID NO: 529 |
| huD08- | X₂ is GGGS, GGGGS, or GGGSGGGS |
| huA02 | SEQ ID NO: 530 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLLESGGGLVQPGGSLRLSCAASGISLDEGVIGWF |
| | RQAPGKEREGVSCISARDGSTHYANAVKGRFTISRDNSKNTVYLQMNSLRAEDT |
| | AVYYCAASLWGLGCKREEDYDYWGQGTLVTVSS |
| 876 | SEQ ID NO: 531 |
| D08-A05 | X₁ is S, GG, AS, ASS or absent (i.e., no amino acid |
| | residues) |
| | X₂ is GGGS, GGGGS, or GGGSGGGS |
| | SEQ ID NO: 532 |
| | X₁ is AS; X₂ is GGGS |
| | X₁QVQLQESGGGLVQAGGSLRLSCAASGRTFSSYGMGWFRQAPGKEREFVAAISW |
| | IGGSTYYADSVKGRFTLSSDNAKNRIALQMNSLKPEDTAVYYCAAKYYTDSYFA |
| | ATSYAYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSS |
| 778 | SEQ ID NO: 533 |
| huD08- | X₂ is GGGS, GGGGS, or GGGSGGGS |
| huA05 | SEQ ID NO: 534 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSS |
| 779 | SEQ ID NO: 535 |
| huD08- | X₂ is GGGS, GGGGS, or GGGSGGGS |
| huA05 | SEQ ID NO: 536 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSS |
| 062 | SEQ ID NO: 537 |
| I04-A02 | X₁ is S, GG, AS, ASS or absent (i.e., no amino acid |
| | residues) |
| | X₂ is GGGS, GGGGS, or GGGSGGGS |
| | SEQ ID NO: 538 |
| | X₁ is AS; X₂ is GGGS |
| | X₁QVQLVESGGGLVQAGGSLRLSCAASGRTFSNYAMAWFRQAPGKEREFVSAINW |
| | FGRATEYADSVKDRETTSGDSAKNTVYLQMNSLKPEDTAVYYCAARTKVITGRR |
| | NQAEYDYWGQGTQVTVSSX₂QVQLQESGGGLVQPGGSLKLSCAASGISLDEGVIG |
| | WFRRAPGKEREGVSCISARDGSTHYANAVKGRFTISRDNTKNTVDLQMNNLEPE |
| | DAGRYYCAASLWGLGCKREEDYDYWGQGTQVTVSS |
| 763 | SEQ ID NO: 539 |
| huI04- | X₂ is GGGS, GGGGS, or GGGSGGGS |
| huA02 | SEQ ID NO: 540 |
| | X₂ is GGGS |
| | EVQLVESGGGLVQPGGSLRLSCAASGRTFSNYAMAWFRQAPGKEREFVSAINWE |
| | GRATEYADSVKDRFTTSGDSSKNTVYLQMNSLRAEDTAVYYCAARTKVTTGRRN |
| | QAEYDYWGQGTLVTVSSX₂EVQLLESGGGLVQPGGSLRLSCAASGISLDEGVIGW |
| | FRQAPGKEREGVSCISARDGSTHYANAVKGRFTISRDNSKNTVYLQMNSLRAED |
| | TAVYYCAASLWGLGCKREEDYDYWGQGTLVTVSS |
| 891 | SEQ ID NO: 541 |
| I04-A05 | X₁ is S, GG, AS, ASS or absent (i.e., no amino acid |
| | residues) |
| | X₂ is GGGS, GGGGS, or GGGSGGGS |
| | SEQ ID NO: 542 |
| | X₁ is AS; X₂ is GGGS |
| | X₁QVQLVESGGGLVQAGGSLRLSCAASGRTFSNYAMAWFRQAPGKEREFVSAINW |
| | FGRATEYADSVKDRFTTSGDSAKNTVYLQMNSLKPEDTAVYYCAARTKVTTGRR |
| | NQAEYDYWGQGTQVTVSSX₂QVQLVESGGGLVQPGGSLRLSCAASGNSLDYPTIG |
| | WFRQAPGKEREGVSCISASDGSTHYADSVKGRFTISRDNTKNTVDLQMNNLKPE |
| | DTGHYYCAASLWGLGCKSEADYDYWGQGTQVTVSS |
| 780 | SEQ ID NO: 543 |
| huI04- | X₂ is GGGS, GGGGS, or GGGSGGGS |
| huA05 | SEQ ID NO: 544 |
| | X₂ is GGGS |
| | EVQLVESGGGLVQPGGSLRLSCAASGRTFSNYAMAWFRQAPGKEREFVSAINWE |
| | GRATEYADSVKDRFTTSGDSSKNTVYLQMNSLRAEDTAVYYCAARTKVTTGRRN |
| | QAEYDYWGQGTLVTVSSX₂EVQLVESGGGLVQPGGSLRLSCAASGNSLDYPTIGW |
| | FRQAPGKGLEGVSAISASDASTHYADAVKGRFTISRDNSKNTVYLQMNSLRAED |
| | TAVYYCAASLHGLGAKSEADYDYWGQGTMVTVSS |
| 781 | SEQ ID NO: 545 |
| huI04- | X₂ is GGGS, GGGGS, or GGGSGGGS |
| huA05 | SEQ ID NO: 546 |
| | X₂ is GGGS |
| | EVQLVESGGGLVQPGGSLRLSCAASGRTFSNYAMAWFRQAPGKEREFVSAINWE |
| | GRATEYADSVKDRETTSGDSSKNTVYLQMNSLRAEDTAVYYCAARTKVTTGRRN |
| | QAEYDYWGQGTLVTVSSX₂EVQLVESGGGLVQPGGSLRLSCAASGNSLDYSTIGW |
| | FRQAPGKGLEGVSCISASEGSTHYADAVKGRFTISRDNSKNTVYLQMNSLRAED |
| | TAVYYCAASLHGLGCKSEADYDYWGQGTMVTVSS |
| 290 | SEQ ID NO: 547 |
| I04-C04 | X₁ is S, GG, AS, ASS or absent (i.e., no amino acid |
| | residues) |
| | X₂ is GGGS, GGGGS, or GGGSGGGS |
| | SEQ ID NO: 548 |
| | X₁ is AS; X₂ is GGGS |
| | X₁QVQLVESGGGLVQAGGSLRLSCAASGRTFSNYAMAWFRQAPGKEREFVSAINW |
| | FGRATEYADSVKDRFTTSGDSAKNTVYLQMNSLKPEDTAVYYCAARTKVTTGRR |
| | NQAEYDYWGQGTQVTVSSX₂QVQLVESGGGLVQSGGSLRLSCVASGFTSPYSTIG |
| | WFRQAPGKEREGVACISSSGRGVNANYIDSVRGRFTISRDNAKNTVYLQMNKLK |
| | PDDTAIYYCATNCWRLTSPAADNWGQGTQVTVSS |
| 791 | SEQ ID NO: 549 |
| huI04- | X₂ is GGGS, GGGGS, or GGGSGGGS |
| huC04 | SEQ ID NO: 550 |
| | X₂ is GGGS |
| | EVQLVESGGGLVQPGGSLRLSCAASGRTFSNYAMAWFRQAPGKEREFVSAINWF |
| | GRATEYADSVKDRETTSGDSSKNTVYLQMNSLRAEDTAVYYCAARTKVTTGRRN |
| | QAEYDYWGQGTLVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSS |
| 892 | SEQ ID NO: 551 |
| J13-A05 | X₁ is S, GG, AS, ASS or absent (i.e., no amino acid |
| | residues) |
| | X₂ is GGGS, GGGGS, or GGGSGGGS |
| | SEQ ID NO: 552 |
| | X₁ is AS; X₂ is GGGS |
| | X₁QVQLVESGGGSVQAGGSLRLSCAASGRTFSSYGMGWFRQAPGKERGEVATINW |
| | IPGNTYYADSVKGRFTISGEEDKNTVYLQMDNLKPEDTAVYYCAARRRIDNYYR |
| | ASEYDYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSS |
| 793 | SEQ ID NO: 553 |
| huJ13- | X₁ is S, GG, AS, ASS or absent (i.e., no amino acid |
| huA05 | residues) |
| | X₂ is GGGS, GGGGS, or GGGSGGGS |
| | SEQ ID NO: 554 |
| | X₁ is AS; X₂ is GGGS |
| | X₁QVQLVESGGGVVQPGGSLRLSCAASGRTFSSYGMGWFRQAPGKEREFVATINW |
| | IPGNTYYADSVKGRFTISGDESKNTVYLQMNSLRAEDTAVYYCAARRRIDNYYR |
| | ASEYDYWGQGTLVTVSSX₂EVQLVESGGGLVQPGGSLRLSCAASGNSLDYPTIGW |
| | FRQAPGKGLEGVSAISASDASTHYADAVKGRFTISRDNSKNTVYLQMNSLRAED |
| | TAVYYCAASLHGLGAKSEADYDYWGQGTMVTVSS |
| 794 | SEQ ID NO: 555 |
| huJ13- | X₁ is S, GG, AS, ASS or absent (i.e., no amino acid |
| huA05 | residues) |
| | X₂ is GGGS, GGGGS, or GGGSGGGS |
| | SEQ ID NO: 556 |
| | X₁ is AS; X₂ is GGGS |
| | X₁QVQLVESGGGVVQPGGSLRLSCAASGRTFSSYGMGWFRQAPGKEREFVATINW |
| | IPGNTYYADSVKGRFTISGDESKNTVYLQMNSLRAEDTAVYYCAARRRIDNYYR |
| | ASEYDYWGQGTLVTVSSX₂EVQLVESGGGLVQPGGSLRLSCAASGNSLDYSTIGW |
| | FRQAPGKGLEGVSCISASEGSTHYADAVKGRFTISRDNSKNTVYLQMNSLRAED |
| | TAVYYCAASLHGLGCKSEADYDYWGQGTMVTVSS |
| 894 | SEQ ID NO: 557 |
| N16-A05 | X₁ is S, GG, AS, ASS or absent (i.e., no amino acid |
| | residues) |
| | X₂ is GGGS, GGGGS, or GGGSGGGS |
| | SEQ ID NO: 558 |
| | X₁ is AS; X₂ is GGGS |
| | X₁QVQLVESGGGLVQAGGSLRLSCAASGGTFSYYGMGWFRQAPGKEREFVAAIRW |
| | IGGDTYYADSVKGRFTISKDNAKNTLYLQMSSLKPEDTAVYYCAAKTSVTSYLS |
| | EKSYNYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSS |
| 795 | SEQ ID NO: 559 |
| huN16- | X₂ is GGGS, GGGGS, or GGGSGGGS |
| huA05 | SEQ ID NO: 560 |
| | X₂ is GGGS |
| | EVQLVESGGGLVQPGGSLRLSCAASGGTFSYYGMGWFRQAPGKEREFVAAIRWI |
| | GGDTYYADSVKGRFTISKDNSKNTLYLQMNSLRAEDTAVYYCAAKTSVTSYLSE |
| | KSYNYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSS |
| 796 | SEQ ID NO: 561 |
| huN16- | X₂ is GGGS, GGGGS, or GGGSGGGS |
| huA05 | SEQ ID NO: 562 |
| | X₂ is GGGS |
| | EVQLVESGGGLVQPGGSLRLSCAASGGTFSYYGMGWFRQAPGKEREFVAAIRWI |
| | GGDTYYADSVKGRFTISKDNSKNTLYLQMNSLRAEDTAVYYCAAKTSVTSYLSE |
| | KSYNYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSS |
| 884 | SEQ ID NO: 563 |
| F09-A05 | X₁ is S, GG, AS, ASS or absent (i.e., no amino acid |
| | residues) |
| | X₂ is GGGS, GGGGS, or GGGSGGGS |
| | SEQ ID NO: 564 |
| | X₁ is AS; X₂ is GGGS |
| | X₁QVQLQESGGGLVQAGGSLSLSCAASGRTFSSYGMGWFRQAPGKEREFVAAISW |
| | IGSATDYADSVRGRFTISGDNAKNTVYLLMNNLKPEDTAVYYCAARRSPMSRTS |
| | VLQYDYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSS |
| 786 | SEQ ID NO: 565 |
| huF09- | X₂ is GGGS, GGGGS, or GGGSGGGS |
| huA05 | SEQ ID NO: 566 |
| | X₂ is GGGS |
| | EVQLLESGGGLVQPGGSLRLSCAASGRTFSSYGMGWFRQAPGKEREFVAAISWI |
| | GSATDYADSVRGRFTISGDNSKNTVYLQMNSLRAEDTAVYYCAARRSPMSRTSV |
| | LQYDYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSS |
| 787 | SEQ ID NO: 567 |
| huF09- | X₂ is GGGS, GGGGS, or GGGSGGGS |
| huA05 | SEQ ID NO: 568 |
| | X₂ is GGGS |
| | EVQLLESGGGLVQPGGSLRLSCAASGRTFSSYGMGWFRQAPGKEREFVAAISWI |
| | GSATDYADSVRGRFTISGDNSKNTVYLQMNSLRAEDTAVYYCAARRSPMSRTSV |
| | LQYDYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSS |
| 058 | SEQ ID NO: 569 |
| F21-A02 | X₁ is S, GG, AS, ASS or absent (i.e., no amino acid |
| | residues) |
| | X₂ is GGGS, GGGGS, or GGGSGGGS |
| | SEQ ID NO: 570 |
| | X₁ is AS; X₂ is GGGS |
| | X₁QVQLVESGGGLVQAGGSLRLSCAASGRTFSSYGMGWFRQAPGKEREFVAAISW |
| | IGNSTYYADSVKGRFTLSGDNAKNTLYLQMNSLKPEDTAVYYCAAKYYTDSYYA |
| | AASYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSS |
| 759 | SEQ ID NO: 571 |
| huF21- | X₂ is GGGS, GGGGS, or GGGSGGGS |
| huA02 | SEQ ID NO: 572 |
| | X₂ is GGGS |
| | EVQLVESGGGLVQPGGSLRLSCAASGRTFSSYGMGWFRQAPGKEREFVAAISWI |
| | GNSTYYADSVKGRFTLSGDNSKNTLYLQMNSLRAEDTAVYYCAAKYYTDSYYAA |
| | ASYAYWGQGTLVTVSSX₂EVQLLESGGGLVQPGGSLRLSCAASGISLDEGVIGWF |
| | RQAPGKEREGVSCISARDGSTHYANAVKGRFTISRDNSKNTVYLQMNSLRAEDT |
| | AVYYCAASLWGLGCKREEDYDYWGQGTLVTVSS |
| 767 | SEQ ID NO: 573 |
| F11-C04 | EX₁QLX₂ESGGGLVQX₃GGSLRLSCAX₄SGRTFSSHTVGWFRQAPGKX₅REFVX₆A |
| con | ITWNGDTTYFGDFVEGRFX₇ISRDX₈X₉KNTLYLX₁₀MNX₁₁LX₁₂X₁₃EDTAVYYCA |
| | AATGQVWSLQYLRESSYAYWGQGTX₁₄VTVSSX₁₅X₁₆VQLVESGGGLVQX₁₇GGSL |
| | RLSCX₁₈ASGFTSPYSTIGWFRQAPGKX₁₉REGVACISSSGRGVNANYIDSVRGRF |
| | TISRDNX₂₀KNTVYLQMNX₂₁LKX₂₂X₂₃DTAX₂₄YYCATNCWRLTSPAADNWGQGT |
| | X₂₅VTVSS |
| | X₁ is L or V |
| | X₂ is L or V |
| | X₃ is A or P |
| | X₄ is A or N |
| | X₅ is E or G |
| | X₆ is A or E |
| | X₇ is S or T |
| | X₈ is N or S |
| | X₉ is A or S |
| | X₁₀ is E or Q |
| | X₁₁ is K or S |
| | X₁₂ is K or R |
| | X₁₃ is A or P |
| | X₁₄ is L or Q |
| | X₁₅ is GGGS, GGGGS, or GGGSGGGS |
| | X₁₆ is E or Q |
| | X₁₇ is P or S |
| | X₁₈ is S or V |
| | X₁₉ is E or G |
| | X₂₀ is A or S |
| | X₂₁ is K or S |
| | X₂₂ is A or P |
| | X₂₃ is D or E |
| | X₂₄ is I or V |
| | X₂₅ is M or Q |
| 284 | SEQ ID NO: 574 |
| F11-C04 | X₂ is GGGS, GGGGS, or GGGSGGGS |
| | SEQ ID NO: 575 |
| | X₂ is GGGS |
| | ELQLVESGGGLVQAGGSLRLSCANSGRTFSSHTVGWFRQAPGKEREFVAAITWN |
| | GDTTYFGDFVEGRESISRDSAKNTLYLEMNKLKPEDTAVYYCAAATGQVWSLQY |
| | LRESSYAYWGQGTQVTVSSX₂QVQLVESGGGLVQSGGSLRLSCVASGFTSPYSTI |
| | GWFRQAPGKEREGVACISSSGRGVNANYIDSVRGRFTISRDNAKNTVYLQMNKL |
| | KPDDTAIYYCATNCWRLTSPAADNWGQGTQVTVSS |
| 183 | SEQ ID NO: 576 |
| huF11- | X₂ is GGGS, GGGGS, or GGGSGGGS |
| huC04 | SEQ ID NO: 577 |
| | X₂ is GGGS |
| | EVQLLESGGGLVQPGGSLRLSCAASGRTFSSHTVGWFRQAPGKGREFVAAITWN |
| | GDTTYFGDFVEGRFTISRDNSKNTLYLQMNSLRAEDTAVYYCAAATGQVWSLQY |
| | LRESSYAYWGQGTLVTVSSX₂EVQLVESGGGLVQPGGSLRLSCSASGFTSPYSTI |
| | GWFRQAPGKGREGVACISSSGRGVNANYIDSVRGRFTISRDNSKNTVYLQMNSL |
| | RAEDTAVYYCATNCWRLTSPAADNWGQGTMVTVSS |
| 184 | SEQ ID NO: 578 |
| huF11- | X₂ is GGGS, GGGGS, or GGGSGGGS |
| huC04 | SEQ ID NO: 579 |
| | X₂ is GGGS |
| | ELQLLESGGGLVQPGGSLRLSCAASGRTFSSHTVGWFRQAPGKGREFVSAITWN |
| | GDTTYFGDFVEGRFTISRDNSKNTLYLQMNSLRAEDTAVYYCAAATGQVWSLQY |
| | LRESSYAYWGQGTLVTVSSX₂EVQLVESGGGLVQPGGSLRLSCSASGFTSPYSTI |
| | GWFRQAPGKGREGVACISSSGRGVNANYIDSVRGRFTISRDNSKNTVYLQMNSL |
| | RAEDTAVYYCATNCWRLTSPAADNWGQGTMVTVSS |
| 239 | SEQ ID NO: 580 |
| G22-B12 | X₁ is S, GG, AS, ASS or absent (i.e., no amino acid |
| | residues) |
| | X₂ is GGGS, GGGGS, or GGGSGGGS |
| | SEQ ID NO: 581 |
| | X₁ is AS; X₂ is GGGS |
| | X₁QLQLVESGGGLVQAGGSLKLSCTASGRTFDGYGMGWFRQAPGKEREFVSAINW |
| | IYGATYYADSVKGRFAVSRDKAKGTMYLQMNSLKPEDTAVYYCAAKINAGSYFN |
| | IGSFGSWGQGTQVTVSSX₂EVQLVESGGGLVQAGGSLRLSCAASGSFSSISTMAW |
| | YRQVPGKQREWLARIIWSSGATNYADSVKGRFTISRDNTKNTMYLQMDRLTPED |
| | TAAYLCSGAISQTRGTFWTYKSQGTQVTVSS |
| 740 | SEQ ID NO: 582 |
| huG22- | X₁ is S, GG, AS, ASS or absent (i.e., no amino acid |
| huB12 | residues) |
| | X₂ is GGGS, GGGGS, or GGGSGGGS |
| | SEQ ID NO: 583 |
| | X₁ is AS; X₂ is GGGS |
| | X₁QLQLVESGGGLVQPGGSLRLSCSASGRTFDGYGMGWFRQAPGKEREFVSAINW |
| | IYGATYYADSVKGRFTVSRDKSKNTMYLQMNSLRAEDTAVYYCAAKINAGSYFN |
| | IGSFGSWGQGTLVTVSSX₂EVQLVESGGGLVQPGGSLRLSCAASGSESSISTMAW |
| | YRQAPGKQREWLARIIWSSGATNYADSVKGRFTISRDNSKNTMYLQMNSLRAED |
| | TAVYYCSGAISQTRGTFWTYKGQGTLVTVSS |
| 286 | SEQ ID NO: 584 |
| G22-C04 | X₁ is S, GG, AS, ASS or absent (i.e., no amino acid |
| | residues) |
| | X₂ is GGGS, GGGGS, or GGGSGGGS |
| | SEQ ID NO: 585 |
| | X₁ is AS; X₂ is GGGS |
| | X₁QLQLVESGGGLVQAGGSLKLSCTASGRTFDGYGMGWFRQAPGKEREFVSAINW |
| | IYGATYYADSVKGRFAVSRDKAKGTMYLQMNSLKPEDTAVYYCAAKINAGSYFN |
| | IGSFGSWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSS |
| 788 | SEQ ID NO: 586 |
| huG22- | X₁ is S, GG, AS, ASS or absent (i.e., no amino acid |
| huC04 | residues) |
| | X₂ is GGGS, GGGGS, or GGGSGGGS |
| | SEQ ID NO: 587 |
| | X₁ is AS; X₂ is GGGS |
| | X₁QLQLVESGGGLVQPGGSLRLSCSASGRTFDGYGMGWFRQAPGKEREFVSAINW |
| | IYGATYYADSVKGRFTVSRDKSKNTMYLQMNSLRAEDTAVYYCAAKINAGSYFN |
| | IGSFGSWGQGTLVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSS |
| 247 | SEQ ID NO: 588 |
| L05-B12 | X₁ is S, GG, AS, ASS or absent (i.e., no amino acid |
| | residues) |
| | X₂ is GGGS, GGGGS, or GGGSGGGS |
| | SEQ ID NO: 589 |
| | X₁ is AS; X₂ is GGGS |
| | X₁QVQLLESGGGLVQAGGSLKLSCTNSGRTFSGYGMGWFRQPPGKEREFVSSINW |
| | IYGATYYSDSVKGRFALSRDKAKNTVYLQMDSLKPEDTAVYYCAAKINGESYFN |
| | TGSFGSWGQGTQVTVSSX₂EVQLVESGGGLVQAGGSLRLSCAASGSFSSISTMAW |
| | YRQVPGKQREWLARIIWSSGATNYADSVKGRFTISRDNTKNTMYLQMDRLTPED |
| | TAAYLCSGAISQTRGTFWTYKSQGTQVTVSS |
| 749 | SEQ ID NO: 590 |
| huL05- | X₂ is GGGS, GGGGS, or GGGSGGGS |
| huB12 | SEQ ID NO: 591 |
| | X₂ is GGGS |
| | EVQLLESGGGLVQPGGSLRLSCAASGRTFSGYGMGWFRQAPGKEREFVSSINWI |
| | YGATYYSDSVKGRFTLSRDKSKNTVYLQMNSLRAEDTAVYYCAAKINGESYFNT |
| | GSFGSWGQGTLVTVSSX₂EVQLVESGGGLVQPGGSLRLSCAASGSFSSISTMAWY |
| | RQAPGKQREWLARIIWSSGATNYADSVKGRFTISRDNSKNTMYLQMNSLRAEDT |
| | AVYYCSGAISQTRGTFWTYKGQGTLVTVSS |

### Fe Domains

In some embodiments, the bispecific antibodies described herein are symmetrical, comprising two identical polypeptide chains. In some embodiments, the bispecific antibodies described herein comprise a first fragment crystallizable (Fc) domain and a second Fc domain that have the same amino acid sequences. Fusion of the tandem VHH1-VHH2 domains with an Fc can increase both serum half-life and potency. In some embodiments, the first Fc domain and the second Fc domain are a human IgG1 or a human IgG4.

In some embodiments, one or both of the first Fc domain and the second Fc domain comprises one or more amino acid substitutions that reduces or inhibits or eliminates antibody-dependent cell-mediated cytotoxicity (ADCC) effector function. In some embodiments, one or both of the first Fc domain and the second Fc domain comprises effector function reducing amino acid substitutions at Fc positions (EU numbering) selected from:
a) alanine at position 234 and alanine at position 235 (234A and 235A);
b) glutamic acid at position 235 (235E);
c) proline at position 228 and glutamic acid at position 235 (228P, 235E);
d) alanine at position 237 (237A);
e) alanine at position 234 and glutamic acid at position 235 (234A and 235E);
f) phenylalanine at position 234, glutamic acid at position 235 and alanine at position 265 (234F, 235E and 265A);
g) phenylalanine at position 234, glutamic acid at position 235 and alanine at position 236 (234F, 235E and 236A);
h) phenylalanine at position 234, glutamic acid at position 235 and arginine at position 236 (234F, 235E and 236R);
i) serine at position 234, threonine at position 235 and arginine at position 236 (234S, 235T and 236R);
j) alanine at position 234, alanine at position 235 and glycine at position 329 (234A, 235A and 329G);
k) alanine at position 234, alanine at position 235 and alanine at position 237 (234A, 235A and 237A);
l) proline at position 228 and glutamic acid at position 235 (228P and 235E);
m) proline at position 228, glutamic acid in position 235 and glycine at position 329 (228P, 235E and 329G);
n) phenylalanine at position 234, glutamic acid at position 235 and serine at position 331 (234F, 235E and 331S);
o) phenylalanine at position 234, glutamine at position 235 and glutamine at position 322 (234F, 235Q and 322Q);
p) alanine at position 233, arginine at position 234 and arginine at position 235 (233A, 234R and 235R);
q) alanine at position 233, aspartic acid at position 234 and glutamic acid at position 235 (233A, 234D and 235E);
r) lysine at position 233, arginine at position 234 and arginine at position 235 (233K, 234R and 235R);
s) alanine at position 234, alanine at position 235, alanine at position 237, serine at position 238, alanine at position 268, serine at position 330 and serine at position 331 (234A, 235A, 237A, 238S, 268A, 330S and 331S); and
t) proline at position 228, alanine at position 234, alanine at position 235, alanine at position 237 and serine at position 238 (228P, 234A, 235A, 237A and 238S).

In some embodiments, one or both of the first Fc domain and the second Fc domain comprises effector function reducing amino acid substitutions at Fc positions (EU numbering) alanine at position 234, alanine at position 235 and alanine at position 237 (234A, 235A and 237A).

In some embodiments, one or both of the first Fc domain and the second Fc domain comprises one or more amino acid substitutions that extend serum half-life. In some embodiments, one or both of the first Fc domain and the second Fc domain comprises serum half-life extending amino acid substitutions at Fc positions (EU numbering) selected from:
a) tyrosine at position 252, threonine at position 254 and glutamic acid at position 256 (252Y, 254T and 256E);
b) leucine at position 428 (428L);
c) glutamine at position 250 and leucine at position 428 (250Q and 428L);
d) leucine at position 428 and serine at position 434 (428L and 434S);
e) leucine at position 428 and alanine at position 434 (428L and 434A);
f) arginine at position 311 and leucine at position 428 (311R and 428L);
g) glycine at position 309 and leucine at position 428 (309G and 428L);
h) glutamine at position 307, valine at position 311 and valine at position 378 (307Q, 311V and 378V); and
i) aspartic acid at position 256, aspartic acid at position 286, arginine at position 307, valine at position 311 and valine at position 378 (256D, 286D, 307R, 311V and 378V).

### Full-Length VHH-VHH-Fc Polypeptides

In some embodiments the bispecific antibody comprises at least one polypeptide (e.g., first and second polypeptides) comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 592-593 and 690. In some embodiments the bispecific antibody comprises at least one polypeptide (e.g., first and second polypeptides) comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 691, 693, 695, 697, 699, 701, 703, 705, and 707. In some embodiments the bispecific antibody comprises at least one polypeptide (e.g., first and second polypeptides) comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 692, 694, 696, 698, 700, 702, 704, 706 and 708.

Generally, the herein described tandem single immunoglobulin domain bispecific (VHH-VHH-Fc) bind to human IL-10Rα/IL-10Rβ complex and cynomolgus IL-10Rα/IL-10Rβ complex, within a 10-fold affinity difference, e.g., within a 5-fold or within a 3-fold affinity difference (e.g., as determined by SPR and expressed as an equilibrium dissociation constant (KD)). Amino acid sequences of illustrative αIL-10Rα/αIL-10Rβ tandem single immunoglobulin domain bispecific (VHH-VHH-Fc) are provided in Table F, below.

The αIL-10Rα-αIL-10Rβ tandem single immunoglobulin domain heavy chains (VHH-VHH-Fc) described herein have a C-terminal lysine. It is possible that the C-terminal lysine might be clipped. Accordingly, in the αIL-10Rα-αIL-10Rβ tandem single immunoglobulin domain heavy chains (VHH-VHH-Fc) described herein, in some embodiments, the C-terminal lysine is present or absent.

TABLE F
Illustrative αIL-10Rα/αIL-10Rβ Tandem Single
Immunoglobulin Domain Bispecific (VHH-VHH-Fc) amino acid sequences

| | |
|---|---|
| VHH-VHH | |
| name | VHH-VHH-Fc amino acid sequence |

αIL-10Rα-αIL-10Rβ Tandem Single Immunoglobulin Domain (VHH-VHH)

| | |
|---|---|
| 774 | SEQ ID NO: 592 |
| E07-A05 | X₁X₂LQLVESGGGLVX₃X₄GX₅SLRLSCAASGRTFSAYGMGWFRQAPGKX₆REFVX₇ |
| con1 | AIRWX₈GX₉X₁₀TYYAX₁₁SVKGRFTISKDX₁₂X₁₃KNTLYLQMX₁₄SLX₁₅X₁₆EDTAV |
| | YYCAAKTX₁₇X₁₈X₁₉SYLSEKSYX₂₀YWGQGTX₂₁VTVSSX₂₂X₂₃VQLX₂₄ESGGGLV |
| | QPGGSLX₂₅LSCAASGX₂₆SLDX₂₇X₂₈X₂₉IGWFRX₃₀APGKX₃₁X₃₂EGVSX₃₃ISA |
| | X₃₄DX₃₅STHYAX₃₆X₃₇VKGRFTISRDNX₃₈KNTVX₃₉LQMNX₄₀LX₄₁X₄₂EDX₄₃X₄₄ |
| | X₄₅YYCAASLX₄₆GLGX₄₇KX₄₈EX₄₉DYDYWGQGTX₅₀VTVSSGSDKTHTCPPCPAP |
| | EAAGAPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAK |
| | GQPREPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK |
| | X₁ is S, GG, AS, ASS or absent (i.e., no amino acid |
| | residues) |
| | X₂ is E or Q |
| | X₃ is Q or R |
| | X₄ is A or P |
| | X₅ is D or G |
| | X₆ is E or G |
| | X₇ is A or S |
| | X₈ is I or Y |
| | X₉ is D or E |
| | X₁₀ is A, G or S |
| | X₁₁ is D or E |
| | X₁₂ is N or S |
| | X₁₃ is A or S |
| | X₁₄ is N or S |
| | X₁₅ is K or R |
| | X₁₆ is A or P |
| | X₁₇ is R or S |
| | X₁₈ is P or V |
| | X₁₉ is D or T |
| | X₂₀ is G or N |
| | X₂₁ is M or Q |
| | X₂₂ is GGGS, GGGGS, or GGGSGGGS |
| | X₂₃ is E or Q |
| | X₂₄ is L or Q |
| | X₂₅ is K or R |
| | X₂₆ is I or N |
| | X₂₇ is E or Y |
| | X₂₈ is G, P or S |
| | X₂₉ is V or T |
| | X₃₀ is Q or R |
| | X₃₁ is E or G |
| | X₃₂ is L or R |
| | X₃₃ is A or C |
| | X₃₄ is R or S |
| | X₃₅ is A or G |
| | X₃₆ is D or N |
| | X₃₇ is A or S |
| | X₃₈ is S or T |
| | X₃₉ is D or Y |
| | X₄₀ is N or S |
| | X₄₁ is K or R |
| | X₄₂ is A or P |
| | X₄₃ is A or T |
| | X₄₄ is A or G |
| | X₄₅ is H or V |
| | X₄₆ is H or W |
| | X₄₇ is A or C |
| | X₄₈ is R or S |
| | X₄₉ is A or E |
| | X₅₀ is L, M or Q |
| 775 | SEQ ID NO: 593 |
| E07-A05 | X₁QLQLVESGGGLVX₃X₄GGSLRLSCAASGRTFSAYGMGWFRQAPGKX₆REFVX₇A |
| con2 | IRWX₈GX₉X₁₀TYYAX₁₁SVKGRFTISKDNX₁₃KNTLYLQMX₁₄SLX₁₅X₁₆EDTAVYY |
| | CAAKTX₁₇X₁₈X₁₉SYLSEKSYX₂₀YWGQGTX₂₁VTVSSX₂₂X₂₃VQLVESGGGLVQPG |
| | GSLRLSCAASGNSLDYPTIGWFRQAPGKX₂₄X₂₅EGVSX₂₆ISASDX₂₇STHYADX₂₈ |
| | VKGRFTISRDNX₂₉KNTVX₃₀LQMNX₃₁LX₃₂X₃₃EDTX₃₄X₃₅YYCAASLX₃₆GLGX₃₇ |
| | KSEADYDYWGQGTX₃₈VTVSSGSDKTHTCPPCPAPEAAGGPSVFLFPPKPKDTLM |
| | ISRTPEVTCVVVDVSHEDPEVKFNWYVDGVEVHNAKTKPREEQYNSTYRVVSVL |
| | TVLHQDWLNGKEYKCKVSNKALPASIEKTISKAKGQPREPQVYTLPPSREEMTK |
| | NQVSLTCLVKGFYPSDIAVEWESNGQPENNYKTTPPVLDSDGSFFLYSKLTVDK |
| | SRWQQGNVFSCSVMHEALHNHYTQKSLSLSPGK |
| | X₁ is S, GG, AS, ASS or absent (i.e., no amino acid |
| | residues) |
| | X₃ is Q or R |
| | X₄ is A or P |
| | X₆ is E or G |
| | X₇ is A or S |
| | X₈ is I or Y |
| | X₉ is D or E |
| | X₁₀ is A, G or S |
| | X₁₁ is D or E |
| | X₁₃ is A or S |
| | X₁₄ is N or S |
| | X₁₅ is K or R |
| | X₁₆ is A or P |
| | X₁₇ is R or S |
| | X₁₈ is P or V |
| | X₁₉ is D or T |
| | X₂₀ is G or N |
| | X₂₁ is M or Q |
| | X₂₂ is GGGS, GGGGS, or GGGSGGGS |
| | X₂₃ is E or Q |
| | X₂₄ is E or G |
| | X₂₅ is L or R |
| | X₂₆ is A or C |
| | X₂₇ is A or G |
| | X₂₈ is A or S |
| | X₂₉ is S or T |
| | X₃₀ is D or Y |
| | X₃₁ is N or S |
| | X₃₂ is K or R |
| | X₃₃ is A or P |
| | X₃₄ is A or G |
| | X₃₅ is H or V |
| | X₃₆ is H or W |
| | X₃₇ is A or C |
| | X₃₈ is M or Q |
| 877 | SEQ ID NO: 594 |
| E07-A05 | X₁ is S, GG, AS, ASS or absent (i.e., no amino acid |
| | residues) |
| | X₂ is GGGS, GGGGS, or GGGSGGGS |
| | SEQ ID NO: 595 |
| | X₁ is AS; X₂ is GGGS |
| | X₁QLQLVESGGGLVRAGGSLRLSCAASGRTFSAYGMGWFRQAPGKEREFVAAIRW |
| | IGDSTYYADSVKGRFTISKDNAKNTLYLQMSSLKPEDTAVYYCAAKTSVTSYLS |
| | EKSYNYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 647 | SEQ ID NO: 596 |
| huE07- | X₁ is S, GG, AS, ASS or absent (i.e., no amino acid |
| huA05 | residues) |
| | X₂ is GGGS, GGGGS, or GGGSGGGS |
| | SEQ ID NO: 597 |
| | X₁ is AS; X₂ is GGGS |
| | X₁QLQLLESGGGLVQPGGSLRLSCAASGRTFSAYGMGWFRQAPGKGREFVSAIRW |
| | YGEGTYYAESVKGRFTISKDNSKNTLYLQMNSLRAEDTAVYYCAAKTRPDSYLS |
| | EKSYGYWGQGTMVTVSSX₂EVQLVESGGGLVQPGGSLRLSCAASGNSLDYPTIGW |
| | FRQAPGKGLEGVSAISASDASTHYADAVKGRFTISRDNSKNTVYLQMNSLRAED |
| | TAVYYCAASLHGLGAKSEADYDYWGQGTMVTVSSGSDKTHTCPPCPAPEAAGAP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 195 | SEQ ID NO:598 |
| huE07- | X₁ is S, GG, AS, ASS or absent (i.e., no amino acid |
| huA05 | residues) |
| | X₂ is GGGS, GGGGS, or GGGSGGGS |
| | SEQ ID NO: 599 |
| | X₁ is AS; X₂ is GGGS |
| | X₁QLQLLESGGGLVQPGGSLRLSCAASGRTFSAYGMGWFRQAPGKGREFVSAIRW |
| | IGDATYYAESVKGRFTISKDNSKNTLYLQMNSLRAEDTAVYYCAAKTSVTSYLS |
| | EKSYNYWGQGTMVTVSSX₂EVQLVESGGGLVQPGGSLRLSCAASGNSLDYSTIGW |
| | FRQAPGKGLEGVSCISASEGSTHYADAVKGRFTISRDNSKNTVYLQMNSLRAED |
| | TAVYYCAASLHGLGCKSEADYDYWGQGTMVTVSSGSDKTHTCPPCPAPEAAGAP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 048 | SEQ ID NO: 600 |
| E07-A02 | X₁ is S, GG, AS, ASS or absent (i.e., no amino acid |
| | residues) |
| | X₂ is GGGS, GGGGS, or GGGSGGGS |
| | SEQ ID NO: 601 |
| | X₁ is AS; X₂ is GGGS |
| | X₁QLQLVESGGGLVRAGGSLRLSCAASGRTFSAYGMGWFRQAPGKEREFVAAIRW |
| | IGDSTYYADSVKGRFTISKDNAKNTLYLQMSSLKPEDTAVYYCAAKTSVTSYLS |
| | EKSYNYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 049 | SEQ ID NO: 602 |
| huE07- | X₁ is S, GG, AS, ASS or absent (i.e., no amino acid |
| huA02 | residues) |
| | X₂ is GGGS, GGGGS, or GGGSGGGS |
| | SEQ ID NO: 603 |
| | X₁ is AS; X₂ is GGGS |
| | X₁QLQLLESGGGLVQPGGSLRLSCAASGRIFSAYGMGWFRQAPGKGREFVSAIRW |
| | YGEGTYYAESVKGRFTISKDNSKNTLYLQMNSLRAEDTAVYYCAAKTRPDSYLS |
| | EKSYGYWGQGTMVTVSSX₂EVQLLESGGGLVQPGGSLRLSCAASGISLDEGVIGW |
| | FRQAPGKEREGVSCISARDGSTHYANAVKGRFTISRDNSKNTVYLQMNSLRAED |
| | TAVYYCAASLWGLGCKREEDYDYWGQGTLVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLIVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 050 | SEQ ID NO: 604 |
| huE07- | X₁ is S, GG, AS, ASS or absent (i.e., no amino acid |
| huA02 | residues) |
| | X₂ is GGGS, GGGGS, or GGGSGGGS |
| | SEQ ID NO: 605 |
| | X₁ is AS; X₂ is GGGS |
| | X₁QLQLLESGGGLVQPGGSLRLSCAASGRTFSAYGMGWFRQAPGKGREFVSAIRW |
| | IGDATYYAESVKGRFTISKDNSKNTLYLQMNSLRAEDTAVYYCAAKTSVTSYLS |
| | EKSYNYWGQGTMVTVSSX₂EVQLLESGGGLVQPGGSLRLSCAASGISLDEGVIGW |
| | FRQAPGKEREGVSCISARDGSTHYANAVKGRFTISRDNSKNTVYLQMNSLRAED |
| | TAVYYCAASLWGLGCKREEDYDYWGQGTLVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 274 | SEQ ID NO: 606 |
| E07-C04 | X₁ is S, GG, AS, ASS or absent (i.e., no amino acid |
| | residues) |
| | X₂ is GGGS, GGGGS, or GGGSGGGS |
| | SEQ ID NO: 607 |
| | X₁ is AS; X₂ is GGGS |
| | X₁QLQLVESGGGLVRAGGSLRLSCAASGRTFSAYGMGWFRQAPGKEREFVAAIRW |
| | IGDSTYYADSVKGRFTISKDNAKNTLYLQMSSLKPEDTAVYYCAAKTSVTSYLS |
| | EKSYNYWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 771 | SEQ ID NO: 608 |
| huE07- | X₁ is S, GG, AS, ASS or absent (i.e., no amino acid |
| huC04 | residues) |
| | X₂ is GGGS, GGGGS, or GGGSGGGS |
| | SEQ ID NO: 609 |
| | X₁ is AS; X₂ is GGGS |
| | X₁QLQLLESGGGLVQPGGSLRLSCAASGRTFSAYGMGWFRQAPGKGREFVSAIRW |
| | YGEGTYYAESVKGRFTISKDNSKNTLYLQMNSLRAEDTAVYYCAAKTRPDSYLS |
| | EKSYGYWGQGTMVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 772 | SEQ ID NO: 610 |
| huE07- | X₁ is S, GG, AS, ASS or absent (i.e., no amino acid |
| huC04 | residues) |
| | X₂ is GGGS, GGGGS, or GGGSGGGS |
| | SEQ ID NO: 611 |
| | X₁ is AS; X₂ is GGGS |
| | X₁QLQLLESGGGLVQPGGSLRLSCAASGRTFSAYGMGWFRQAPGKGREFVSAIRW |
| | IGDATYYAESVKGRFTISKDNSKNTLYLQMNSLRAEDTAVYYCAAKTSVTSYLS |
| | EKSYNYWGQGTMVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 054 | SEQ ID NO: 612 |
| E15-A02 | X₁ is S, GG, AS, ASS or absent (i.e., no amino acid |
| | residues) |
| | X₂ is GGGS, GGGGS, or GGGSGGGS |
| | SEQ ID NO: 613 |
| | X₁ is AS; X₂ is GGGS |
| | X₁QVQLQESGGGLVQAGGSLRLSCAASGRTFSSYGMGWFRQAPGKEREFVAAISW |
| | IGNSAYYADSVKGRFTLSGDNAKNTLYLQMNSLKPEDTAVYYCAAKYYTDSYYA |
| | AASYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKITPPVLDS |
| | DGSFFLYSKLTVDKSRWQQGNVFSCSVMHEALHNHYTQKSLSLSPGK |
| 755 | SEQ ID NO: 614 |
| huE15- | X₂ is GGGS, GGGGS, or GGGSGGGS |
| huA02 | SEQ ID NO: 615 |
| | X₂ is GGGS |
| | EVQLLESGGGLVQPGGSLRLSCAASGRTFSSYGMGWFRQAPGKEREFVAAISWI |
| | GNSAYYADSVKGRFTLSGDNSKNTLYLQMNSLRAEDTAVYYCAAKYYTDSYYAA |
| | ASYAYWGQGTLVTVSSX₂EVQLLESGGGLVQPGGSLRLSCAASGISLDEGVIGWE |
| | RQAPGKEREGVSCISARDGSTHYANAVKGRFTISRDNSKNTVYLQMNSLRAEDT |
| | AVYYCAASLWGLGCKREEDYDYWGQGTLVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 883 | SEQ ID NO: 616 |
| E15-A05 | X₁ is S, GG, AS, ASS or absent (i.e., no amino acid |
| | residues) |
| | X₂ is GGGS, GGGGS, or GGGSGGGS |
| | SEQ ID NO: 617 |
| | X₁ is AS; X₂ is GGGS |
| | X₁QVQLQESGGGLVQAGGSLRLSCAASGRTFSSYGMGWFRQAPGKEREFVAAISW |
| | IGNSAYYADSVKGRFTLSGDNAKNTLYLQMNSLKPEDTAVYYCAAKYYTDSYYA |
| | AASYAYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 784 | SEQ ID NO: 618 |
| huE15- | X₂ is GGGS, GGGGS, or GGGSGGGS |
| huA05 | SEQ ID NO: 619 |
| | X₂ is GGGS |
| | EVQLLESGGGLVQPGGSLRLSCAASGRTFSSYGMGWFRQAPGKEREFVAAISWI |
| | GNSAYYADSVKGRFTLSGDNSKNTLYLQMNSLRAEDTAVYYCAAKYYTDSYYAA |
| | ASYAYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 785 | SEQ ID NO: 620 |
| E15- | X₂ is GGGS, GGGGS, or GGGSGGGS |
| huA05 | SEQ ID NO: 621 |
| | X₂ is GGGS |
| | EVQLLESGGGLVQPGGSLRLSCAASGRTFSSYGMGWFRQAPGKEREFVAAISWI |
| | GNSAYYADSVKGRFTLSGDNSKNTLYLQMNSLRAEDTAVYYCAAKYYTDSYYAA |
| | ASYAYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 046 | SEQ ID NO: 622 |
| A06-A02 | X₁ is S, GG, AS, ASS or absent (i.e., no amino acid |
| | residues) |
| | X₂ is GGGS, GGGGS, or GGGSGGGS |
| | SEQ ID NO: 623 |
| | X₁ is AS; X₂ is GGGS |
| | X₁QLQLVESGGGLVQAGGSLRLSCAASGRTFSSYGMGWFRQAPGKERGLVAAINW |
| | IPGTTYYADSVKGRFTISGDKDKNTLYLQMDNLKPEDTAVYYCAARRRIDNYYR |
| | ASEYDYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLIVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 747 | SEQ ID NO: 624 |
| huA06- | X₂ is GGGS, GGGGS, or GGGSGGGS |
| huA02 | SEQ ID NO: 625 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLLESGGGLVQPGGSLRLSCAASGISLDEGVIGWF |
| | RQAPGKEREGVSCISARDGSTHYANAVKGRFTISRDNSKNTVYLQMNSLRAEDT |
| | AVYYCAASLWGLGCKREEDYDYWGQGTLVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 875 | SEQ ID NO: 626 |
| A06-A05 | X₁ is S, GG, AS, ASS or absent (i.e., no amino acid |
| | residues) |
| | X₂ is GGGS, GGGGS, or GGGSGGGS |
| | SEQ ID NO: 627 |
| | X₁ is AS; X₂ is GGGS |
| | X₁QLQLVESGGGLVQAGGSLRLSCAASGRTFSSYGMGWFRQAPGKERGLVAAINW |
| | IPGTTYYADSVKGRFTISGDKDKNTLYLQMDNLKPEDTAVYYCAARRRIDNYYR |
| | ASEYDYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 776 | SEQ ID NO: 628 |
| huA06- | X₂ is GGGS, GGGGS, or GGGSGGGS |
| huA05 | SEQ ID NO: 629 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 777 | SEQ ID NO: 630 |
| huA06- | X₂ is GGGS, GGGGS, or GGGSGGGS |
| huA05 | SEQ ID NO: 631 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 272 | SEQ ID NO: 632 |
| A06-C04 | X₁ is S, GG, AS, ASS or absent (i.e., no amino acid |
| | residues) |
| | X₂ is GGGS, GGGGS, or GGGSGGGS |
| | SEQ ID NO: 633 |
| | X₁ is AS; X₂ is GGGS |
| | X₁QLQLVESGGGLVQAGGSLRLSCAASGRTFSSYGMGWFRQAPGKERGLVAAINW |
| | IPGTTYYADSVKGRFTISGDKDKNTLYLQMDNLKPEDTAVYYCAARRRIDNYYR |
| | ASEYDYWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 773 | SEQ ID NO: 634 |
| huA06- | X₂ is GGGS, GGGGS, or GGGSGGGS |
| huC04 | SEQ ID NO: 635 |
| | X₂ is GGGS |
| | ELQLVESGGGLVQPGGSLRLSCAASGRTFSSYGMGWFRQAPGKERELVAAINWI |
| | PGTTYYADSVKGRFTISGDKSKNTLYLQMNSLRAEDTAVYYCAARRRIDNYYRA |
| | SEYDYWGQGTLVTVSSX₂EVQLVESGGGLVQPGGSLRLSCSASGFTSPYSTIGWF |
| | RQAPGKGREGVACISSSGRGVNANYIDSVRGRFTISRDNSKNTVYLQMNSLRAE |
| | DTAVYYCATNCWRLTSPAADNWGQGTMVTVSSGSDKTHTCPPCPAPEAAGGPSV |
| | FLFPPKPKDTLMISRTPEVTCVVVDVSHEDPEVKFNWYVDGVEVHNAKTKPREE |
| | QYNSTYRVVSVLTVLHQDWLNGKEYKCKVSNKALPASIEKTISKAKGQPREPQV |
| | YTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 052 | SEQ ID NO: 636 |
| C09-A02 | X₁ is S, GG, AS, ASS or absent (i.e., no amino acid |
| | residues) |
| | X₂ is GGGS, GGGGS, or GGGSGGGS |
| | SEQ ID NO: 637 |
| | X₁ is AS; X₂ is GGGS |
| | X₁QVQLQESGGGLVQAGGSLRLSCAASGRTFSSYGMGWFRQAPGKEREFVAAISW |
| | IGGGTYYADSVKGRFTLSRDNAKNMVYLQMNSLKPEDTAVYYCAAKYYPDSYFA |
| | AASYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 753 | SEQ ID NO: 638 |
| huC09- | X₂ is GGGS, GGGGS, or GGGSGGGS |
| huA02 | SEQ ID NO: 639 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLLESGGGLVQPGGSLRLSCAASGISLDEGVIGWF |
| | RQAPGKEREGVSCISARDGSTHYANAVKGRFTISRDNSKNTVYLQMNSLRAEDT |
| | AVYYCAASLWGLGCKREEDYDYWGQGTLVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 278 | SEQ ID NO: 640 |
| C09-C04 | X₁ is S, GG, AS, ASS or absent (i.e., no amino acid |
| | residues) |
| | X₂ is GGGS, GGGGS, or GGGSGGGS |
| | SEQ ID NO: 641 |
| | X₁ is AS; X₂ is GGGS |
| | X₁QVQLQESGGGLVQAGGSLRLSCAASGRTFSSYGMGWFRQAPGKEREFVAAISW |
| | IGGGTYYADSVKGRFTLSRDNAKNMVYLQMNSLKPEDTAVYYCAAKYYPDSYFA |
| | AASYAYWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 769 | SEQ ID NO: 642 |
| | X₂ is GGGS, GGGGS, or GGGSGGGS |
| huC09- | SEQ ID NO: 643 |
| huC04 | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLVESGGGLVQPGGSLRLSCSASGFTSPYSTIGWF |
| | RQAPGKGREGVACISSSGRGVNANYIDSVRGRFTISRDNSKNTVYLQMNSLRAE |
| | DTAVYYCATNCWRLISPAADNWGQGTMVTVSSGSDKTHTCPPCPAPEAAGGPSV |
| | FLFPPKPKDTLMISRTPEVTCVVVDVSHEDPEVKFNWYVDGVEVHNAKTKPREE |
| | QYNSTYRVVSVLIVLHQDWLNGKEYKCKVSNKALPASIEKTISKAKGQPREPQV |
| | YTLPPSREEMTKNQVSLTCLVKGFYPSDIAVEWESNGQPENNYKTTPPVLDSDG |
| | SFFLYSKLTVDKSRWQQGNVFSCSVMHEALHNHYTQKSLSLSPGK |
| 047 | SEQ ID NO: 644 |
| D08-A02 | X₁ is S, GG, AS, ASS or absent (i.e., no amino acid |
| | residues) |
| | X₂ is GGGS, GGGGS, or GGGSGGGS |
| | SEQ ID NO: 645 |
| | X₁ is AS; X₂ is GGGS |
| | X₁QVQLQESGGGLVQAGGSLRLSCAASGRTFSSYGMGWFRQAPGKEREFVAAISW |
| | IGGSTYYADSVKGRFTLSSDNAKNRIALQMNSLKPEDTAVYYCAAKYYTDSYFA |
| | ATSYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 748 | SEQ ID NO: 646 |
| huD08- | X₂ is GGGS, GGGGS, or GGGSGGGS |
| huA02 | SEQ ID NO: 647 |
| | X₂ is GGGS |
| | EVQLLESGGGLVQPGGSLRLSCAASGRTFSSYGMGWERQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLLESGGGLVQPGGSLRLSCAASGISLDEGVIGWE |
| | RQAPGKEREGVSCISARDGSTHYANAVKGRFTISRDNSKNTVYLQMNSLRAEDT |
| | AVYYCAASLWGLGCKREEDYDYWGQGTLVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 876 | SEQ ID NO: 648 |
| D08-A05 | X₁ is S, GG, AS, ASS or absent (i.e., no amino acid |
| | residues) |
| | X₂ is GGGS, GGGGS, or GGGSGGGS |
| | SEQ ID NO: 649 |
| | X₁ is AS; X₂ is GGGS |
| | X₁QVQLQESGGGLVQAGGSLRLSCAASGRTFSSYGMGWFRQAPGKEREFVAAISW |
| | IGGSTYYADSVKGRFTLSSDNAKNRIALQMNSLKPEDTAVYYCAAKYYTDSYFA |
| | ATSYAYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 778 | SEQ ID NO: 650 |
| huD08- | X₂ is GGGS, GGGGS, or GGGSGGGS |
| huA05 | SEQ ID NO: 651 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 779 | SEQ ID NO: 652 |
| huD08- | X₂ is GGGS, GGGGS, or GGGSGGGS |
| huA05 | SEQ ID NO: 653 |
| | X₂ is GGGS |
| | EVQLLESGGGLVQPGGSLRLSCAASGRTFSSYGMGWFRQAPGKEREFVAAISWI |
| | GGGTYYADSVKGRFTLSRDNSKNTVYLQMNSLRAEDTAVYYCAAKYYPDSYFAA |
| | ASYAYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 062 | SEQ ID NO: 654 |
| I04-A02 | X₁ is S, GG, AS, ASS or absent (i.e., no amino acid |
| | residues) |
| | X₂ is GGGS, GGGGS, or GGGSGGGS |
| | SEQ ID NO: 655 |
| | X₁ is AS; X₂ is GGGS |
| | X₁QVQLVESGGGLVQAGGSLRLSCAASGRTFSNYAMAWFRQAPGKEREFVSAINW |
| | FGRATEYADSVKDRFTTSGDSAKNTVYLQMNSLKPEDTAVYYCAARTKVITGRR |
| | NQAEYDYWGQGTQVTVSSX₂QVQLQESGGGLVQPGGSLKLSCAASGISLDEGVIG |
| | WFRRAPGKEREGVSCISARDGSTHYANAVKGRFTISRDNTKNTVDLQMNNLEPE |
| | DAGRYYCAASLWGLGCKREEDYDYWGQGTQVTVSSGSDKTHTCPPCPAPEAAGG |
| | PSVFLFPPKPKDTLMISRTPEVTCVVVDVSHEDPEVKFNWYVDGVEVHNAKTKP |
| | REEQYNSTYRVVSVLIVLHQDWLNGKEYKCKVSNKALPASIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| 763 | SEQ ID NO: 656 |
| huI04- | X₂ is GGGS, GGGGS, or GGGSGGGS |
| huA02 | SEQ ID NO: 657 |
| | X₂ is GGGS |
| | EVQLVESGGGLVQPGGSLRLSCAASGRTFSNYAMAWFRQAPGKEREFVSAINWF |
| | GRATEYADSVKDRFTTSGDSSKNTVYLQMNSLRAEDTAVYYCAARTKVITGRRN |
| | QAEYDYWGQGTLVTVSSX₂EVQLLESGGGLVQPGGSLRLSCAASGISLDEGVIGW |
| | FRQAPGKEREGVSCISARDGSTHYANAVKGRFTISRDNSKNTVYLQMNSLRAED |
| | TAVYYCAASLWGLGCKREEDYDYWGQGTLVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLIVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 891 | SEQ ID NO: 658 |
| I04-A05 | X₁ is S, GG, AS, ASS or absent (i.e., no amino acid |
| | residues) |
| | X₂ is GGGS, GGGGS, or GGGSGGGS |
| | SEQ ID NO: 659 |
| | X₁ is AS; X₂ is GGGS |
| | X₁QVQLVESGGGLVQAGGSLRLSCAASGRTFSNYAMAWERQAPGKEREFVSAINW |
| | FGRATEYADSVKDRFTTSGDSAKNTVYLQMNSLKPEDTAVYYCAARTKVTTGRR |
| | NQAEYDYWGQGTQVTVSSX₂QVQLVESGGGLVQPGGSLRLSCAASGNSLDYPTIG |
| | WFRQAPGKEREGVSCISASDGSTHYADSVKGRFTISRDNTKNTVDLQMNNLKPE |
| | DTGHYYCAASLWGLGCKSEADYDYWGQGTQVTVSSGSDKTHTCPPCPAPEAAGG |
| | PSVFLFPPKPKDTLMISRTPEVTCVVVDVSHEDPEVKFNWYVDGVEVHNAKTKP |
| | REEQYNSTYRVVSVLIVLHQDWLNGKEYKCKVSNKALPASIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| 780 | SEQ ID NO: 660 |
| huI04- | X₂ is GGGS, GGGGS, or GGGSGGGS |
| huA05 | SEQ ID NO: 661 |
| | X₂ is GGGS |
| | EVQLVESGGGLVQPGGSLRLSCAASGRTFSNYAMAWFRQAPGKEREFVSAINWF |
| | GRATEYADSVKDRFTTSGDSSKNTVYLQMNSLRAEDTAVYYCAARTKVITGRRN |
| | QAEYDYWGQGTLVTVSSX₂EVQLVESGGGLVQPGGSLRLSCAASGNSLDYPTIGW |
| | FRQAPGKGLEGVSAISASDASTHYADAVKGRFTISRDNSKNIVYLQMNSLRAED |
| | TAVYYCAASLHGLGAKSEADYDYWGQGTMVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 781 | SEQ ID NO: 662 |
| huI04- | X₂ is GGGS, GGGGS, or GGGSGGGS |
| huA05 | SEQ ID NO: 663 |
| | X₂ is GGGS |
| | EVQLVESGGGLVQPGGSLRLSCAASGRTFSNYAMAWERQAPGKEREFVSAINWE |
| | GRATEYADSVKDRFTTSGDSSKNTVYLQMNSLRAEDTAVYYCAARTKVTTGRRN |
| | QAEYDYWGQGTLVTVSSX₂EVQLVESGGGLVQPGGSLRLSCAASGNSLDYSTIGW |
| | FRQAPGKGLEGVSCISASEGSTHYADAVKGRFTISRDNSKNIVYLQMNSLRAED |
| | TAVYYCAASLHGLGCKSEADYDYWGQGTMVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 290 | SEQ ID NO: 664 |
| I04-C04 | X₁ is S, GG, AS, ASS or absent (i.e., no amino acid |
| | residues) |
| | X₂ is GGGS, GGGGS, or GGGSGGGS |
| | SEQ ID NO: 665 |
| | X₁ is AS; X₂ is GGGS |
| | X₁QVQLVESGGGLVQAGGSLRLSCAASGRTFSNYAMAWFRQAPGKEREFVSAINW |
| | FGRATEYADSVKDRETTSGDSAKNTVYLQMNSLKPEDTAVYYCAARTKVITGRR |
| | NQAEYDYWGQGTQVTVSSX₂QVQLVESGGGLVQSGGSLRLSCVASGFTSPYSTIG |
| | WFRQAPGKEREGVACISSSGRGVNANYIDSVRGRFTISRDNAKNTVYLQMNKLK |
| | PDDTAIYYCATNCWRLTSPAADNWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 791 | SEQ ID NO: 666 |
| huI04- | X₂ is GGGS, GGGGS, or GGGSGGGS |
| huC04 | SEQ ID NO: 667 |
| | X₂ is GGGS |
| | EVQLVESGGGLVQPGGSLRLSCAASGRTFSNYAMAWFRQAPGKEREFVSAINWE |
| | GRATEYADSVKDRFTTSGDSSKNTVYLQMNSLRAEDTAVYYCAARTKVTTGRRN |
| | QAEYDYWGQGTLVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 892 | SEQ ID NO: 668 |
| J13-A05 | X₁ is S, GG, AS, ASS or absent (i.e., no amino acid |
| | residues) |
| | X₂ is GGGS, GGGGS, or GGGSGGGS |
| | SEQ ID NO: 669 |
| | X₁ is AS; X₂ is GGGS |
| | X₁QVQLVESGGGSVQAGGSLRLSCAASGRTFSSYGMGWFRQAPGKERGEVATINW |
| | IPGNTYYADSVKGRFTISGEEDKNTVYLQMDNLKPEDTAVYYCAARRRIDNYYR |
| | ASEYDYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 793 | SEQ ID NO: 670 |
| huJ13- | X₁ is S, GG, AS, ASS or absent (i.e., no amino acid |
| huA05 | residues) |
| | X₂ is GGGS, GGGGS, or GGGSGGGS |
| | SEQ ID NO: 671 |
| | X₁ is AS; X₂ is GGGS |
| | X₁QVQLVESGGGVVQPGGSLRLSCAASGRTFSSYGMGWFRQAPGKEREFVATINW |
| | IPGNTYYADSVKGRFTISGDESKNTVYLQMNSLRAEDTAVYYCAARRRIDNYYR |
| | ASEYDYWGQGTLVTVSSX₂EVQLVESGGGLVQPGGSLRLSCAASGNSLDYPTIGW |
| | FRQAPGKGLEGVSAISASDASTHYADAVKGRFTISRDNSKNTVYLQMNSLRAED |
| | TAVYYCAASLHGLGAKSEADYDYWGQGTMVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 794 | SEQ ID NO: 672 |
| huJ13- | X₁ is S, GG, AS, ASS or absent (i.e., no amino acid |
| huA05 | residues) |
| | X₂ is GGGS, GGGGS, or GGGSGGGS |
| | SEQ ID NO: 673 |
| | X₁ is AS; X₂ is GGGS |
| | X₁QVQLVESGGGVVQPGGSLRLSCAASGRTFSSYGMGWERQAPGKEREFVATINW |
| | IPGNTYYADSVKGRFTISGDESKNTVYLQMNSLRAEDTAVYYCAARRRIDNYYR |
| | ASEYDYWGQGTLVTVSSX₂EVQLVESGGGLVQPGGSLRLSCAASGNSLDYSTIGW |
| | FRQAPGKGLEGVSCISASEGSTHYADAVKGRFTISRDNSKNTVYLQMNSLRAED |
| | TAVYYCAASLHGLGCKSEADYDYWGQGTMVTVSSGSDKTHTCPPCPAPEAAGGP |
| | SVFLFPPKPKDILMISRTPEVTCVVVDVSHEDPEVKFNWYVDGVEVHNAKTKPR |
| | EEQYNSTYRVVSVLIVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 894 | SEQ ID NO: 674 |
| N16-A05 | X₁ is S, GG, AS, ASS or absent (i.e., no amino acid |
| | residues) |
| | X₂ is GGGS, GGGGS, or GGGSGGGS |
| | SEQ ID NO: 675 |
| | X₁ is AS; X₂ is GGGS |
| | X₁QVQLVESGGGLVQAGGSLRLSCAASGGTFSYYGMGWFRQAPGKEREFVAAIRW |
| | IGGDTYYADSVKGRFTISKDNAKNTLYLQMSSLKPEDTAVYYCAAKTSVTSYLS |
| | EKSYNYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 795 | SEQ ID NO: 676 |
| huN16- | X₂ is GGGS, GGGGS, or GGGSGGGS |
| huA05 | SEQ ID NO: 677 |
| | X₂ is GGGS |
| | EVQLVESGGGLVQPGGSLRLSCAASGGTFSYYGMGWFRQAPGKEREFVAAIRWI |
| | GGDTYYADSVKGRFTISKDNSKNTLYLQMNSLRAEDTAVYYCAAKTSVTSYLSE |
| | KSYNYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 796 | SEQ ID NO: 678 |
| huN16- | X₂ is GGGS, GGGGS, or GGGSGGGS |
| huA05 | SEQ ID NO: 679 |
| | X₂ is GGGS |
| | EVQLVESGGGLVQPGGSLRLSCAASGGTFSYYGMGWFRQAPGKEREFVAAIRWI |
| | GGDTYYADSVKGRFTISKDNSKNTLYLQMNSLRAEDTAVYYCAAKTSVTSYLSE |
| | KSYNYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 884 | SEQ ID NO: 680 |
| F09-A05 | X₁ is S, GG, AS, ASS or absent (i.e., no amino acid |
| | residues) |
| | X₂ is GGGS, GGGGS, or GGGSGGGS |
| | SEQ ID NO: 681 |
| | X₁ is AS; X₂ is GGGS |
| | X₁QVQLQESGGGLVQAGGSLSLSCAASGRTFSSYGMGWFRQAPGKEREFVAAISW |
| | IGSATDYADSVRGRFTISGDNAKNTVYLLMNNLKPEDTAVYYCAARRSPMSRTS |
| | VLQYDYWGQGTQVTVSSX₂QVQLVESGGGLVQPGGSLRLSCAASGNSLDYPTIGW |
| | FRQAPGKEREGVSCISASDGSTHYADSVKGRFTISRDNTKNTVDLQMNNLKPED |
| | TGHYYCAASLWGLGCKSEADYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 786 | SEQ ID NO: 682 |
| huF09- | X₂ is GGGS, GGGGS, or GGGSGGGS |
| huA05 | SEQ ID NO: 683 |
| | X₂ is GGGS |
| | EVQLLESGGGLVQPGGSLRLSCAASGRTFSSYGMGWFRQAPGKEREFVAAISWI |
| | GSATDYADSVRGRFTISGDNSKNTVYLQMNSLRAEDTAVYYCAARRSPMSRTSV |
| | LQYDYWGQGTLVTVSSX₂EVQLVESGGGLVQPGGSLRLSCAASGNSLDYPTIGWF |
| | RQAPGKGLEGVSAISASDASTHYADAVKGRFTISRDNSKNTVYLQMNSLRAEDT |
| | AVYYCAASLHGLGAKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 787 | SEQ ID NO: 684 |
| huF09- | X₂ is GGGS, GGGGS, or GGGSGGGS |
| huA05 | SEQ ID NO: 685 |
| | X₂ is GGGS |
| | EVQLLESGGGLVQPGGSLRLSCAASGRTFSSYGMGWFRQAPGKEREFVAAISWI |
| | GSATDYADSVRGRFTISGDNSKNTVYLQMNSLRAEDTAVYYCAARRSPMSRTSV |
| | LQYDYWGQGTLVTVSSX₂EVQLVESGGGLVQPGGSLRLSCAASGNSLDYSTIGWF |
| | RQAPGKGLEGVSCISASEGSTHYADAVKGRFTISRDNSKNTVYLQMNSLRAEDT |
| | AVYYCAASLHGLGCKSEADYDYWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 058 | SEQ ID NO: 686 |
| F21-A02 | X₁ is S, GG, AS, ASS or absent (i.e., no amino acid |
| | residues) |
| | X₂ is GGGS, GGGGS, or GGGSGGGS |
| | SEQ ID NO: 687 |
| | X₁ is AS; X₂ is GGGS |
| | X₁QVQLVESGGGLVQAGGSLRLSCAASGRTFSSYGMGWFRQAPGKEREFVAAISW |
| | IGNSTYYADSVKGRFTLSGDNAKNTLYLQMNSLKPEDTAVYYCAAKYYTDSYYA |
| | AASYAYWGQGTQVTVSSX₂QVQLQESGGGLVQPGGSLKLSCAASGISLDEGVIGW |
| | FRRAPGKEREGVSCISARDGSTHYANAVKGRFTISRDNTKNTVDLQMNNLEPED |
| | AGRYYCAASLWGLGCKREEDYDYWGQGTQVTVSSGSDKTHTCPPCPAPEAAGGP |
| | SVFLFPPKPKDTLMISRTPEVTCVVVDVSHEDPEVKFNWYVDGVEVHNAKTKPR |
| | EEQYNSTYRVVSVLTVLHQDWLNGKEYKCKVSNKALPASIEKTISKAKGQPREP |
| | QVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 759 | SEQ ID NO: 688 |
| huF21- | X₂ is GGGS, GGGGS, or GGGSGGGS |
| huA02 | SEQ ID NO: 689 |
| | X₂ is GGGS |
| | EVQLVESGGGLVQPGGSLRLSCAASGRTFSSYGMGWFRQAPGKEREFVAAISWI |
| | GNSTYYADSVKGRFTLSGDNSKNTLYLQMNSLRAEDTAVYYCAAKYYTDSYYAA |
| | ASYAYWGQGTLVTVSSX₂EVQLLESGGGLVQPGGSLRLSCAASGISLDEGVIGWE |
| | RQAPGKEREGVSCISARDGSTHYANAVKGRFTISRDNSKNTVYLQMNSLRAEDT |
| | AVYYCAASLWGLGCKREEDYDYWGQGTLVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 767 | SEQ ID NO: 690 |
| F11-C04 | EX₁QLX₂ESGGGLVQX₃GGSLRLSCAX₄SGRTFSSHTVGWFRQAPGKX₅REFVX₆A |
| con | ITWNGDTTYFGDFVEGRFX₇ISRDX₈X₉KNTLYLX₁₀MNX₁₁LX₁₂X₁₃EDTAVYYCA |
| | AATGQVWSLQYLRESSYAYWGQGTX₁₄VTVSSX₁₅X₁₆VQLVESGGGLVQX₁₇GGSL |
| | RLSCX₁₈ASGFTSPYSTIGWFRQAPGKX₁₉REGVACISSSGRGVNANYIDSVRGRE |
| | TISRDNX₂₀KNTVYLQMNX₂₁LKX₂₂X₂₃DTAX₂₄YYCATNCWRLTSPAADNWGQGT |
| | X₂₅VTVSSGSDKTHTCPPCPAPEAAGGPSVFLFPPKPKDTLMISRTPEVTCVVVD |
| | VSHEDPEVKFNWYVDGVEVHNAKTKPREEQYNSTYRVVSVLTVLHQDWLNGKEY |
| | KCKVSNKALPASIEKTISKAKGQPREPQVYTLPPSREEMTKNQVSLTCLVKGFY |
| | PSDIAVEWESNGQPENNYKTTPPVLDSDGSFFLYSKLTVDKSRWQQGNVFSCSV |
| | MHEALHNHYTQKSLSLSPGK |
| | X₁ is L or V |
| | X₂ is L or V |
| | X₃ is A or P |
| | X₄ is A or N |
| | X₅ is E or G |
| | X₆ is A or E |
| | X₇ is S or T |
| | X₈ is N or S |
| | X₉ is A or S |
| | X₁₀ is E or Q |
| | X₁₁ is K or S |
| | X₁₂ is K or R |
| | X₁₃ is A or P |
| | X₁₄ is L or Q |
| | X₁₅ is GGGS, GGGGS, or GGGSGGGS |
| | X₁₆ is E or Q |
| | X₁₇ is P or S |
| | X₁₈ is S or V |
| | X₁₉ is E or G |
| | X₂₀ is A or S |
| | X₂₁ is K or S |
| | X₂₂ is A or P |
| | X₂₃ is D or E |
| | X₂₄ is I or V |
| | X₂₅ is M or Q |
| 284 | SEQ ID NO: 691 |
| F11-C04 | X₂ is GGGS, GGGGS, or GGGSGGGS |
| | SEQ ID NO: 692 |
| | X₂ is GGGS |
| | ELQLVESGGGLVQAGGSLRLSCANSGRTFSSHTVGWFRQAPGKEREFVAAITWN |
| | GDTTYFGDFVEGRFSISRDSAKNTLYLEMNKLKPEDTAVYYCAAATGQVWSLQY |
| | LRESSYAYWGQGTQVTVSSX₂QVQLVESGGGLVQSGGSLRLSCVASGFTSPYSTI |
| | GWFRQAPGKEREGVACISSSGRGVNANYIDSVRGRFTISRDNAKNTVYLQMNKL |
| | KPDDTAIYYCATNCWRLTSPAADNWGQGTQVTVSSGSDKTHTCPPCPAPEAAGG |
| | PSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPASIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| 183 | SEQ ID NO: 693 |
| huF11- | X₂ is GGGS, GGGGS, or GGGSGGGS |
| huC04 | SEQ ID NO: 694 |
| | X₂ is GGGS |
| | EVQLLESGGGLVQPGGSLRLSCAASGRTFSSHTVGWFRQAPGKGREFVAAITWN |
| | GDTTYFGDFVEGRFTISRDNSKNTLYLQMNSLRAEDTAVYYCAAATGQVWSLQY |
| | LRESSYAYWGQGTLVTVSSX₂EVQLVESGGGLVQPGGSLRLSCSASGFTSPYSTI |
| | GWFRQAPGKGREGVACISSSGRGVNANYIDSVRGRFTISRDNSKNTVYLQMNSL |
| | RAEDTAVYYCATNCWRLTSPAADNWGQGTMVTVSSGSDKTHTCPPCPAPEAAGA |
| | PSVFLFPPKPKDTLMISRTPEVTCVVVDVSHEDPEVKFNWYVDGVEVHNAKTKP |
| | REEQYNSTYRVVSVLIVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| 184 | SEQ ID NO: 695 |
| huF11- | X₂ is GGGS, GGGGS, or GGGSGGGS |
| huC04 | SEQ ID NO: 696 |
| | X₂ is GGGS |
| | ELQLLESGGGLVQPGGSLRLSCAASGRTFSSHTVGWFRQAPGKGREFVSAITWN |
| | GDTTYFGDFVEGRFTISRDNSKNTLYLQMNSLRAEDTAVYYCAAATGQVWSLQY |
| | LRESSYAYWGQGTLVTVSSX₂EVQLVESGGGLVQPGGSLRLSCSASGFTSPYSTI |
| | GWFRQAPGKGREGVACISSSGRGVNANYIDSVRGRFTISRDNSKNIVYLQMNSL |
| | RAEDTAVYYCATNCWRLISPAADNWGQGTMVTVSSGSDKTHTCPPCPAPEAAGA |
| | PSVFLFPPKPKDTLMISRTPEVTCVVVDVSHEDPEVKFNWYVDGVEVHNAKTKP |
| | REEQYNSTYRVVSVLIVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| 239 | SEQ ID NO: 697 |
| G22-B12 | X₁ is S, GG, AS, ASS or absent (i.e., no amino acid |
| | residues) |
| | X₂ is GGGS, GGGGS, or GGGSGGGS |
| | SEQ ID NO: 698 |
| | X₁ is AS; X₂ is GGGS |
| | X₁QLQLVESGGGLVQAGGSLKLSCTASGRTFDGYGMGWFRQAPGKEREFVSAINW |
| | IYGATYYADSVKGRFAVSRDKAKGTMYLQMNSLKPEDTAVYYCAAKINAGSYEN |
| | IGSFGSWGQGTQVTVSSX₂EVQLVESGGGLVQAGGSLRLSCAASGSFSSISTMAW |
| | YRQVPGKQREWLARIIWSSGATNYADSVKGRFTISRDNTKNTMYLQMDRLTPED |
| | TAAYLCSGAISQTRGTFWTYKSQGTQVTVSSGSDKTHTCPPCPAPEAAGGPSVE |
| | LFPPKPKDTLMISRTPEVTCVVVDVSHEDPEVKFNWYVDGVEVHNAKTKPREEQ |
| | YNSTYRVVSVLTVLHQDWLNGKEYKCKVSNKALPASIEKTISKAKGQPREPQVY |
| | TLPPSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 740 | SEQ ID NO: 699 |
| huG22- | X₁ is S, GG, AS, ASS or absent (i.e., no amino acid |
| huB12 | residues) |
| | X₂ is GGGS, GGGGS, or GGGSGGGS |
| | SEQ ID NO: 700 |
| | X₁ is AS; X₂ is GGGS |
| | X₁QLQLVESGGGLVQPGGSLRLSCSASGRTFDGYGMGWFRQAPGKEREFVSAINW |
| | IYGATYYADSVKGRFTVSRDKSKNTMYLQMNSLRAEDTAVYYCAAKINAGSYEN |
| | IGSFGSWGQGTLVTVSSX₂EVQLVESGGGLVQPGGSLRLSCAASGSESSISTMAW |
| | YRQAPGKQREWLARIIWSSGATNYADSVKGRFTISRDNSKNTMYLQMNSLRAED |
| | TAVYYCSGAISQTRGTFWTYKGQGTLVTVSSGSDKTHTCPPCPAPEAAGGPSVF |
| | LFPPKPKDTLMISRTPEVTCVVVDVSHEDPEVKFNWYVDGVEVHNAKTKPREEQ |
| | YNSTYRVVSVLTVLHQDWLNGKEYKCKVSNKALPASIEKTISKAKGQPREPQVY |
| | TLPPSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 247 | SEQ ID NO: 701 |
| L05-B12 | X₁ is S, GG, AS, ASS or absent (i.e., no amino acid |
| | residues) |
| | X₂ is GGGS, GGGGS, or GGGSGGGS |
| | SEQ ID NO: 702 |
| | X₁ is AS; X₂ is GGGS |
| | X₁QVQLLESGGGLVQAGGSLKLSCTNSGRTFSGYGMGWFRQPPGKEREFVSSINW |
| | IYGATYYSDSVKGRFALSRDKAKNTVYLQMDSLKPEDTAVYYCAAKINGESYEN |
| | TGSFGSWGQGTQVTVSSX₂EVQLVESGGGLVQAGGSLRLSCAASGSESSISTMAW |
| | YRQVPGKQREWLARIIWSSGATNYADSVKGRFTISRDNTKNTMYLQMDRLTPED |
| | TAAYLCSGAISQTRGTFWTYKSQGTQVTVSSGSDKTHTCPPCPAPEAAGGPSVE |
| | LFPPKPKDTLMISRTPEVTCVVVDVSHEDPEVKFNWYVDGVEVHNAKTKPREEQ |
| | YNSTYRVVSVLTVLHQDWLNGKEYKCKVSNKALPASIEKTISKAKGQPREPQVY |
| | TLPPSREEMTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 748 | SEQ ID NO: 703 |
| huL05- | X₂ is GGGS, GGGGS, or GGGSGGGS |
| huB12 | SEQ ID NO: 704 |
| | X₂ is GGGS |
| | EVQLLESGGGLVQPGGSLRLSCAASGRTFSGYGMGWFRQAPGKEREFVSSINWI |
| | YGATYYSDSVKGRFTLSRDKSKNTVYLQMNSLRAEDTAVYYCAAKINGESYENT |
| | GSFGSWGQGTLVTVSSX₂EVQLVESGGGLVQPGGSLRLSCAASGSFSSISTMAWY |
| | RQAPGKQREWLARIIWSSGATNYADSVKGRFTISRDNSKNTMYLQMNSLRAEDT |
| | AVYYCSGAISQTRGTFWTYKGQGTLVTVSSGSDKTHTCPPCPAPEAAGGPSVEL |
| | FPPKPKDTLMISRTPEVTCVVVDVSHEDPEVKFNWYVDGVEVHNAKTKPREEQY |
| | NSTYRVVSVLTVLHQDWLNGKEYKCKVSNKALPASIEKTISKAKGQPREPQVYT |
| | LPPSREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGK |
| 286 | SEQ ID NO: 705 |
| G22-C04 | X₁ is S, GG, AS, ASS or absent (i.e., no amino acid |
| | residues) |
| | X₂ is GGGS, GGGGS, or GGGSGGGS |
| | SEQ ID NO: 706 |
| | X₁ is AS; X₂ is GGGS |
| | X₁QLQLVESGGGLVQAGGSLKLSCTASGRTFDGYGMGWFRQAPGKEREFVSAINW |
| | IYGATYYADSVKGRFAVSRDKAKGTMYLQMNSLKPEDTAVYYCAAKINAGSYEN |
| | IGSFGSWGQGTQVTVSSX₂QVQLVESGGGLVQSGGSLRLSCVASGFTSPYSTIGW |
| | FRQAPGKEREGVACISSSGRGVNANYIDSVRGRFTISRDNAKNTVYLQMNKLKP |
| | DDTAIYYCATNCWRLTSPAADNWGQGTQVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 788 | SEQ ID NO: 707 |
| huG22- | X₁ is S, GG, AS, ASS or absent (i.e., no amino acid |
| huC04 | residues) |
| | X₂ is GGGS, GGGGS, or GGGSGGGS |
| | SEQ ID NO: 708 |
| | X₁ is AS; X₂ is GGGS |
| | X₁QLQLVESGGGLVQPGGSLRLSCSASGRTEDGYGMGWFRQAPGKEREFVSAINW |
| | IYGATYYADSVKGRFTVSRDKSKNTMYLQMNSLRAEDTAVYYCAAKINAGSYEN |
| | IGSFGSWGQGTLVTVSSX₂EVQLVESGGGLVQPGGSLRLSCSASGFTSPYSTIGW |
| | FRQAPGKGREGVACISSSGRGVNANYIDSVRGRFTISRDNSKNTVYLQMNSLRA |
| | EDTAVYYCATNCWRLTSPAADNWGQGTMVTVSSGSDKTHTCPPCPAPEAAGGPS |
| | VFLFPPKPKDTLMISRTPEVTCVVVDVSHEDPEVKFNWYVDGVEVHNAKTKPRE |
| | EQYNSTYRVVSVLIVLHQDWLNGKEYKCKVSNKALPASIEKTISKAKGQPREPQ |
| | VYTLPPSREEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |

### Bispecific E07-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 22, 23, 24, 291, 292 and 293; (877; according to Kabat)
2) SEQ ID NOs: 22, 25, 26, 291, 294 and 295; (647; according to Kabat)
3) SEQ ID NOs: 22, 27, 24, 291, 297 and 298; (195; according to Kabat)
4) SEQ ID NOs: 77, 78, 79, 329, 330 and 331; (877; according to IMGT)
5) SEQ ID NOs: 77, 80, 81, 329, 332 and 333; (647; according to IMGT)
6) SEQ ID NOs: 77, 82, 79, 334, 335 and 336; (195; according to IMGT)
7) SEQ ID NOs: 127, 128, 129, 369, 370 and 371; (877; according to Chothia)
8) SEQ ID NOs: 127, 130, 131, 369, 372 and 373; (647; according to Chothia)
9) SEQ ID NOs: 127, 130, 131, 374, 375 and 376; (195; according to Chothia)
10) SEQ ID NOs: 174, 175, 176, 409, 410 and 411; (877; according to Honegger)
11) SEQ ID NOs: 174, 177, 178, 409, 412 and 413; (647; according to Honegger) or
12) SEQ ID NOs: 174, 179, 176, 414, 415 and 416; (195; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 233 and 448; [E07con-A05con] or
2) SEQ ID NOs: 234 and 449; [E07con-A05con].

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 235 and 450; (877)
2) SEQ ID NOs: 237 and 451; (647)
3) SEQ ID NOs: 239 and 452; (195)
4) SEQ ID NOs: 236 and 450; (877)
5) SEQ ID NOs: 238 and 451; (647) or
6) SEQ ID NOs: 240 and 452; (195).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 475-482 and 737-751. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 592-599 (bispecific antibodies 195, 647 and 877; E07-A05 and humanized and consensus versions thereof).

### Bispecific E07-A02

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 22, 23, 24, 288, 289 and 290; (048; according to Kabat)
2) SEQ ID NOs: 22, 25, 26, 288, 289 and 290; (049; according to Kabat)
3) SEQ ID NOs: 22, 27, 24, 288, 289 and 290; (050; according to Kabat)
4) SEQ ID NOs: 77, 78, 79, 326, 327 and 328; (048; according to IMGT)
5) SEQ ID NOs: 77, 80, 81, 326, 327 and 328; (049; according to IMGT)
6) SEQ ID NOs: 77, 82, 79, 326, 327 and 328; (050; according to IMGT)
7) SEQ ID NOs: 127, 128, 129, 366, 367 and 368; (048; according to Chothia)
8) SEQ ID NOs: 127, 130, 131, 366, 367 and 368; (049; according to Chothia)
9) SEQ ID NOs: 127, 130, 131, 366, 367 and 368; (050; according to Chothia)
10) SEQ ID NOs: 174, 175, 176, 406, 407 and 408; (048; according to Honegger)
11) SEQ ID NOs: 174, 177, 178, 406, 407 and 408; (049; according to Honegger) or
12) SEQ ID NOs: 174, 179, 176, 406, 407 and 408; (050; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 235 and 446; (048)
2) SEQ ID NOs: 237 and 447; (049)
3) SEQ ID NOs: 239 and 447; (050)
4) SEQ ID NOs: 236 and 446; (048)
5) SEQ ID NOs: 238 and 447; (049) or
6) SEQ ID NOs: 240 and 447; (050).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 483-488. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 600-605 (bispecific antibodies 048, 049 and 050; E07-A02 and humanized and consensus versions thereof).

### Bispecific E07-C04

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 22, 23, 24, 296, 308 and 309; (274; according to Kabat)
2) SEQ ID NOs: 22, 25, 26, 296, 308 and 309; (771; according to Kabat)
3) SEQ ID NOs: 22, 27, 24, 296, 308 and 309; (772; according to Kabat)
4) SEQ ID NOs: 77, 78, 79, 346, 347 and 348; (274; according to IMGT)
5) SEQ ID NOs: 77, 80, 81, 346, 347 and 348; (771; according to IMGT)
6) SEQ ID NOs: 77, 82, 79, 346, 347 and 348; (772; according to IMGT)
7) SEQ ID NOs: 127, 128, 129, 386, 387 and 388; (274; according to Chothia)
8) SEQ ID NOs: 127, 130, 131, 386, 387 and 388; (771; according to Chothia)
9) SEQ ID NOs: 127, 130, 131, 386, 387 and 388; (772; according to Chothia)
10) SEQ ID NOs: 174, 175, 176, 426, 427 and 428; (274; according to Honegger)
11) SEQ ID NOs: 174, 177, 178, 426, 427 and 428; (771; according to Honegger) or
12) SEQ ID NOs: 174, 179, 176, 426, 427 and 428; (772; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 233 and 460; [E07con-C04con] or
2) SEQ ID NOs: 234 and 460; [E07con-C04con].

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 235 and 461; (274)
2) SEQ ID NOs: 237 and 462; (771)
3) SEQ ID NOs: 239 and 462; (772)
4) SEQ ID NOs: 236 and 461; (274)
5) SEQ ID NOs: 238 and 462; (771) or
6) SEQ ID NOs: 240 and 462; (772).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 489-494. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 606-611 (bispecific antibodies 274, 771 and 772; E07-C04 and humanized and consensus versions thereof).

### Bispecific E15-A02

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 28, 29, 288, 289 and 290; (054; according to Kabat)
2) SEQ ID NOs: 59, 83, 84, 326, 327 and 328; (054; according to IMGT)
3) SEQ ID NOs: 111, 132, 133, 366, 367 and 368; (054; according to Chothia) or
4) SEQ ID NOs: 156, 180, 181, 406, 407 and 408; (054; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 241 and 446; (054)
2) SEQ ID NOs: 243 and 447; (755)
3) SEQ ID NOs: 242 and 446; (054) or
4) SEQ ID NOs: 243 and 447; (755).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 495-498. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 612-615 (bispecific antibodies 054 and 755; E15-A02 and humanized and consensus versions thereof).

### Bispecific E15-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 28, 29, 291, 294 and 295; (883; according to Kabat)
2) SEQ ID NOs: 4, 28, 29, 291, 294 and 295; (784; according to Kabat)
3) SEQ ID NOs: 4, 28, 29, 291, 297 and 298; (785; according to Kabat)
4) SEQ ID NOs: 59, 83, 84, 329, 330 and 331; (883; according to IMGT)
5) SEQ ID NOs: 59, 83, 84, 329, 332 and 333; (784; according to IMGT)
6) SEQ ID NOs: 59, 83, 84, 334, 335 and 336; (785; according to IMGT)
7) SEQ ID NOs: 111, 132, 133, 369, 370 and 371; (883; according to Chothia)
8) SEQ ID NOs: 111, 132, 133, 369, 372 and 373; (784; according to Chothia)
9) SEQ ID NOs: 111, 132, 133, 374, 375 and 376; (785; according to Chothia)
10) SEQ ID NOs: 156, 180, 181, 409, 410 and 411; (883; according to Honegger)
11) SEQ ID NOs: 156, 180, 181, 409, 412 and 413; (784; according to Honegger) or
12) SEQ ID NOs: 156, 180, 181, 414, 415 and 416; (785; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 241 and 450; (883)
2) SEQ ID NOs: 243 and 451; (784)
3) SEQ ID NOs: 243 and 452; (785)
4) SEQ ID NOs: 242 and 450; (883)
5) SEQ ID NOs: 243 and 451; (784) or
6) SEQ ID NOs: 243 and 452; (785).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 499-504. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 616-621 (bispecific antibodies 784-785 and 883; E15-A05 and humanized and consensus versions thereof).

### Bispecific A06-A02

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 5, 6, 288, 289 and 290; (046; according to Kabat)
2) SEQ ID NOs: 59, 60, 61, 326, 327 and 328; (046; according to IMGT)
3) SEQ ID NOs: 111, 112, 113, 366, 367 and 368; (046; according to Chothia) or
4) SEQ ID NOs: 156, 157, 158, 406, 407 and 408; (046; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 213 and 446; (046)
2) SEQ ID NOs: 215 and 447; (747)
3) SEQ ID NOs: 214 and 446; (046) or
4) SEQ ID NOs: 215 and 447; (747).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 505-508. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 622-625 (bispecific antibodies 046 and 747; A06-A02 and humanized and consensus versions thereof).

### Bispecific A06-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 5, 6, 291, 294 and 295; (875; according to Kabat)
2) SEQ ID NOs: 4, 5, 6, 291, 294 and 295; (776; according to Kabat)
3) SEQ ID NOs: 4, 5, 6, 291, 297 and 298; (777; according to Kabat)
4) SEQ ID NOs: 59, 60, 61, 329, 330 and 331; (875; according to IMGT)
5) SEQ ID NOs: 59, 60, 61, 329, 332 and 333; (776; according to IMGT)
6) SEQ ID NOs: 59, 60, 61, 334, 335 and 336; (777; according to IMGT)
7) SEQ ID NOs: 111, 112, 113, 369, 370 and 371; (875; according to Chothia)
8) SEQ ID NOs: 111, 112, 113, 369, 372 and 373; (776; according to Chothia)
9) SEQ ID NOs: 111, 112, 113, 374, 375 and 376; (777; according to Chothia)
10) SEQ ID NOs: 156, 157, 158, 409, 410 and 411; (875; according to Honegger)
11) SEQ ID NOs: 156, 157, 158, 409, 412 and 413; (776; according to Honegger) or
12) SEQ ID NOs: 156, 157, 158, 414, 415 and 416; (777; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 212 and 448; [A06con-A05con] or
2) SEQ ID NOs: 212 and 449; [A06con-A05con].

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 213 and 450; (875)
2) SEQ ID NOs: 215 and 451; (776)
3) SEQ ID NOs: 215 and 452; (777)
4) SEQ ID NOs: 214 and 450; (875)
5) SEQ ID NOs: 215 and 451; (776) or
6) SEQ ID NOs: 215 and 452; (777).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 509-514. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 626-631 (bispecific antibodies 875 and 776-777; A06-A05 and humanized and consensus versions thereof).

### Bispecific A06-C04

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 5, 6, 296, 308 and 309; (272; according to Kabat)
2) SEQ ID NOs: 59, 60, 61, 346, 347 and 348; (272; according to IMGT)
3) SEQ ID NOs: 111, 112, 113, 386, 387 and 388; (272; according to Chothia) or
4) SEQ ID NOs: 156, 157, 158, 426, 427 and 428; (272; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in SEQ ID NOs: 212 and 460; [A06con-C04con].

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 213 and 461; (272)
2) SEQ ID NOs: 215 and 462; (773)
3) SEQ ID NOs: 214 and 461; (272) or
4) SEQ ID NOs: 215 and 462; (773).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 515-518. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 632-635 (bispecific antibodies 272 and 773; A06-C04 and humanized and consensus versions thereof).

### Bispecific C09-A02

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 15, 16, 288, 289 and 290; (052; according to Kabat)
2) SEQ ID NOs: 59, 70, 71, 326, 327 and 328; (052; according to IMGT)
3) SEQ ID NOs: 111, 121, 122, 366, 367 and 368; (052; according to Chothia) or
4) SEQ ID NOs: 156, 167, 168, 406, 407 and 408; (052; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 224 and 446; (052)
2) SEQ ID NOs: 226 and 447; (753)
3) SEQ ID NOs: 225 and 446; (052) or
4) SEQ ID NOs: 226 and 447; (753).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 519-522. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 636-639 (bispecific antibodies 052 and 753; C09-A02 and humanized and consensus versions thereof).

### Bispecific C09-C04

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 15, 16, 296, 308 and 309; (278; according to Kabat)
2) SEQ ID NOs: 59, 70, 71, 346, 347 and 348; (278; according to IMGT)
3) SEQ ID NOs: 111, 121, 122, 386, 387 and 388; (278; according to Chothia) or
4) SEQ ID NOs: 156, 167, 168, 426, 427 and 428; (278; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in SEQ ID NOs: 710 and 460; [C09con-C04con].

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 224 and 461; (278)
2) SEQ ID NOs: 226 and 462; (769)
3) SEQ ID NOs: 225 and 461; (278) or
4) SEQ ID NOs: 226 and 462; (769).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 523-526. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 640-643 (bispecific antibodies 278 and 769; C09-C04 and humanized and consensus versions thereof).

### Bispecific D08-A02

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 17, 18, 288, 289 and 290; (047; according to Kabat)
2) SEQ ID NOs: 59, 72, 73, 326, 327 and 328; (047; according to IMGT)
3) SEQ ID NOs: 111, 121, 123, 366, 367 and 368; (047; according to Chothia) or
4) SEQ ID NOs: 156, 169, 170, 406, 407 and 408; (047; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 227 and 446; (047)
2) SEQ ID NOs: 229 and 447; (748)
3) SEQ ID NOs: 228 and 446; (047) or
4) SEQ ID NOs: 229 and 447; (748).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 527-530. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 644-647 (bispecific antibodies 047 and 748; D08-A02 and humanized and consensus versions thereof).

### Bispecific D08-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 17, 18, 291, 292 and 293; (876; according to Kabat)
2) SEQ ID NOs: 4, 17, 18, 291, 294 and 295; (778; according to Kabat)
3) SEQ ID NOs: 4, 17, 18, 291, 297 and 298; (779; according to Kabat)
4) SEQ ID NOs: 59, 72, 73, 329, 330 and 331; (876; according to IMGT)
5) SEQ ID NOs: 59, 72, 73, 329, 332 and 333; (778; according to IMGT)
6) SEQ ID NOs: 59, 72, 73, 334, 335 and 336; (779; according to IMGT)
7) SEQ ID NOs: 111, 121, 123, 369, 370 and 371; (876; according to Chothia)
8) SEQ ID NOs: 111, 121, 123, 369, 372 and 373; (778; according to Chothia)
9) SEQ ID NOs: 111, 121, 123, 374, 375 and 376; (779; according to Chothia)
10) SEQ ID NOs: 156, 169, 170, 409, 410 and 411; (876; according to Honegger)
11) SEQ ID NOs: 156, 169, 170, 409, 412 and 413; (778; according to Honegger) or
12) SEQ ID NOs: 156, 169, 170, 414, 415 and 416; (779; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 227 and 450; (876)
2) SEQ ID NOs: 229 and 451; (778)
3) SEQ ID NOs: 229 and 452; (779)
4) SEQ ID NOs: 228 and 450; (876)
5) SEQ ID NOs: 229 and 451; (778) or
6) SEQ ID NOs: 229 and 452; (779).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 531-536. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 648-653 (bispecific antibodies 876 and 778-779; D08-A05 and humanized and consensus versions thereof).

### Bispecific I04-A02

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 45, 46, 47, 288, 289 and 290; (062; according to Kabat)
2) SEQ ID NOs: 98, 99, 100, 326, 327 and 328; (062; according to IMGT)
3) SEQ ID NOs: 145, 146, 147, 366, 367 and 368; (062; according to Chothia) or
4) SEQ ID NOs: 198, 199, 200, 406, 407 and 408; (062; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 267 and 446; (062)
2) SEQ ID NOs: 269 and 447; (763)
3) SEQ ID NOs: 268 and 446; (062) or
4) SEQ ID NOs: 269 and 447; (763).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 537-540. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 654-657 (bispecific antibodies 062 and 763; I04-A02 and humanized and consensus versions thereof).

### Bispecific I04-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 45, 46, 47, 291, 292 and 293; (891; according to Kabat)
2) SEQ ID NOs: 45, 46, 47, 291, 294 and 295; (780; according to Kabat)
3) SEQ ID NOs: 45, 46, 47, 291, 297 and 298; (781; according to Kabat)
4) SEQ ID NOs: 98, 99, 100, 329, 330 and 331; (891; according to IMGT)
5) SEQ ID NOs: 98, 99, 100, 329, 332 and 333; (780; according to IMGT)
6) SEQ ID NOs: 98, 99, 100, 334, 335 and 336; (781; according to IMGT)
7) SEQ ID NOs: 145, 146, 147, 369, 370 and 371; (891; according to Chothia)
8) SEQ ID NOs: 145, 146, 147, 369, 372 and 373; (780; according to Chothia)
9) SEQ ID NOs: 145, 146, 147, 374, 375 and 376; (781; according to Chothia)
10) SEQ ID NOs: 198, 199, 200, 409, 410 and 411; (891; according to Honegger)
11) SEQ ID NOs: 198, 199, 200, 409, 412 and 413; (780; according to Honegger) or
12) SEQ ID NOs: 198, 199, 200, 414, 415 and 416; (781; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 267 and 450; (891)
2) SEQ ID NOs: 269 and 451; (780)
3) SEQ ID NOs: 269 and 452; (781)
4) SEQ ID NOs: 268 and 450; (891)
5) SEQ ID NOs: 269 and 451; (780) or
6) SEQ ID NOs: 269 and 452; (781).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 541-546. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 658-663 (bispecific antibodies 891 and 780-781; I04-A05 and humanized and consensus versions thereof).

### Bispecific I04-C04

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 45, 46, 47, 296, 308 and 309; (290; according to Kabat)
2) SEQ ID NOs: 98, 99, 100, 326, 327 and 328; (290; according to IMGT)
3) SEQ ID NOs: 145, 146, 147, 386, 387 and 388; (290; according to Chothia) or
4) SEQ ID NOs: 198, 199, 200, 426, 427 and 428; (290; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:

1) SEQ ID NOs: 267 and 461; (290)
2) SEQ ID NOs: 269 and 462; (791)
3) SEQ ID NOs: 268 and 461; (290) or
4) SEQ ID NOs: 269 and 462; (791).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 547-550. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 664-667 (bispecific antibodies 290 and 791; I04-C04 and humanized and consensus versions thereof).

### Bispecific J13-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 48, 6, 291, 292 and 293; (892; according to Kabat)
2) SEQ ID NOs: 4, 48, 6, 291, 294 and 295; (793; according to Kabat)
3) SEQ ID NOs: 4, 48, 6, 291, 297 and 298; (794; according to Kabat)
4) SEQ ID NOs: 59, 101, 61, 329, 330 and 331; (892; according to IMGT)
5) SEQ ID NOs: 59, 101, 61, 329, 332 and 333; (793; according to IMGT)
6) SEQ ID NOs: 59, 101, 61, 334, 335 and 336; (794; according to IMGT)
7) SEQ ID NOs: 111, 112, 113, 369, 370 and 371; (892; according to Chothia)
8) SEQ ID NOs: 111, 112, 113, 369, 372 and 373; (793; according to Chothia)
9) SEQ ID NOs: 111, 112, 113, 374, 375 and 376; (794; according to Chothia)
10) SEQ ID NOs: 156, 201, 158, 409, 410 and 411; (892; according to Honegger)
11) SEQ ID NOs: 156, 201, 158, 409, 412 and 413; (793; according to Honegger) or
12) SEQ ID NOs: 156, 201, 158, 414, 415 and 416; (794; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 270 and 450; (892)
2) SEQ ID NOs: 272 and 451; (793)
3) SEQ ID NOs: 272 and 452; (794)
4) SEQ ID NOs: 271 and 450; (892)
5) SEQ ID NOs: 273 and 451; (793) or
6) SEQ ID NOs: 274 and 452; (794).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 551-556. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 668-673 (bispecific antibodies 892 and 793-794; J13-A05 and humanized and consensus versions thereof).

### Bispecific N16-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 54, 55, 24, 291, 292 and 293; (894; according to Kabat)
2) SEQ ID NOs: 54, 55, 24, 291, 294 and 295; (795; according to Kabat)
3) SEQ ID NOs: 54, 55, 24, 291, 297 and 298; (796; according to Kabat)
4) SEQ ID NOs: 106, 107, 79, 329, 330 and 331; (894; according to IMGT)
5) SEQ ID NOs: 106, 107, 79, 329, 332 and 333; (795; according to IMGT)
6) SEQ ID NOs: 106, 107, 79, 334, 335 and 336; (796; according to IMGT)
7) SEQ ID NOs: 152, 121, 129, 369, 370 and 371; (894; according to Chothia)
8) SEQ ID NOs: 152, 121, 129, 369, 372 and 373; (795; according to Chothia)
9) SEQ ID NOs: 152, 121, 129, 374, 375 and 376; (796; according to Chothia)
10) SEQ ID NOs: 208, 209, 176, 409, 410 and 411; (894; according to Honegger)
11) SEQ ID NOs: 208, 209, 176, 409, 412 and 413; (795; according to Honegger) or
12) SEQ ID NOs: 208, 209, 176, 414, 415 and 416; (796; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 285 and 450; (894)
2) SEQ ID NOs: 287 and 451; (795)
3) SEQ ID NOs: 287 and 452; (796)
4) SEQ ID NOs: 286 and 450; (894)
5) SEQ ID NOs: 287 and 451; (795) or
6) SEQ ID NOs: 287 and 452; (796).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 557-562. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 674-679 (bispecific antibodies 894 and 795-796; N16-A05 and humanized and consensus versions thereof).

### Bispecific F09-A05

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 30, 31, 291, 292 and 293; (884; according to Kabat)
2) SEQ ID NOs: 4, 30, 31, 291, 294 and 295; (786; according to Kabat)
3) SEQ ID NOs: 4, 30, 31, 291, 297 and 298; (787; according to Kabat)
4) SEQ ID NOs: 59, 85, 86, 329, 330 and 331; (884; according to IMGT)
5) SEQ ID NOs: 59, 85, 86, 329, 332 and 333; (786; according to IMGT)
6) SEQ ID NOs: 59, 85, 86, 334, 335 and 336; (787; according to IMGT)
7) SEQ ID NOs: 111, 134, 135, 369, 370 and 371; (884; according to Chothia)
8) SEQ ID NOs: 111, 134, 135, 369, 372 and 373; (786; according to Chothia)
9) SEQ ID NOs: 111, 134, 135, 374, 375 and 376; (787; according to Chothia)
10) SEQ ID NOs: 156, 182, 183, 409, 410 and 411; (884; according to Honegger)
11) SEQ ID NOs: 156, 182, 183, 409, 412 and 413; (786; according to Honegger) or
12) SEQ ID NOs: 156, 182, 183, 414, 415 and 416; (787; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 244 and 450; (884)
2) SEQ ID NOs: 246 and 451; (786)
3) SEQ ID NOs: 247 and 452; (787)
4) SEQ ID NOs: 245 and 450; (884)
5) SEQ ID NOs: 246 and 451; (786) or
6) SEQ ID NOs: 246 and 452; (787).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 563-568. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 680-685 (bispecific antibodies 884 and 786-787; F09-A05 and humanized and consensus versions thereof).

### Bispecific F21-A02

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 4, 36, 29, 288, 289 and 290; (058; according to Kabat)
2) SEQ ID NOs: 59, 91, 84, 326, 327 and 328; (058; according to IMGT)
3) SEQ ID NOs: 111, 132, 133, 366, 367 and 368; (058; according to Chothia) or
4) SEQ ID NOs: 156, 188, 181, 406, 407 and 408; (058; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 253 and 446; (058)
2) SEQ ID NOs: 255 and 447; (759)
3) SEQ ID NOs: 254 and 446; (058) or
4) SEQ ID NOs: 255 and 447; (759).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 569-572. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 686-689 (bispecific antibodies 058 and 759; F21-A02 and humanized and consensus versions thereof).

### Bispecific F11-C04

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 33, 34, 35, 296, 308 and 309; (284; according to Kabat)
2) SEQ ID NOs: 88, 89, 90, 326, 327 and 328; (284; according to IMGT)
3) SEQ ID NOs: 137, 138, 139, 386, 387 and 388; (284; according to Chothia)
4) SEQ ID NOs: 185, 186, 187, 426, 427 and 428; (284; according to Honegger)
5) SEQ ID NOs: 709, 186, 187, 426, 427 and 428; (183; according to Honegger) or
6) SEQ ID NOs: 709, 186, 187, 426, 427 and 428; (184; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in SEQ ID NOs: 249 and 460; [F11con-C04con].

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 250 and 461; (284)
2) SEQ ID NOs: 251 and 462; (183)
3) SEQ ID NOs: 252 and 462; (184)
4) SEQ ID NOs: 250 and 461; (284)
5) SEQ ID NOs: 251 and 462; (183) or
6) SEQ ID NOs: 252 and 462; (184).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 573-579 and 757-758. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 690-696 (bispecific antibodies 183-184, 284 and 767; F11-C04 and humanized and consensus versions thereof).

### Bispecific G22-B12

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 19, 37, 38, 302, 303 and 304; (239; according to Kabat)
2) SEQ ID NOs: 92, 75, 93, 340, 341 and 342; (239; according to IMGT)
3) SEQ ID NOs: 140, 125, 141, 380, 381 and 381; (239; according to Chothia) or
4) SEQ ID NOs: 189, 190, 191, 420, 421 and 422; (239; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 256 and 456; (239)
2) SEQ ID NOs: 258 and 457; (740)
3) SEQ ID NOs: 257 and 456; (239) or
4) SEQ ID NOs: 259 and 457; (740).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 580-583. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 697-700 (bispecific antibodies 239 and 740; G22-B12 and humanized and consensus versions thereof).

### Bispecific G22-C04

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 19, 37, 38, 296, 308 and 309; (286; according to Kabat)
2) SEQ ID NOs: 92, 75, 93, 326, 327 and 328; (286; according to IMGT)
3) SEQ ID NOs: 140, 125, 141, 386, 387 and 388; (286; according to Chothia) or
4) SEQ ID NOs: 189, 190, 191, 426, 427 and 428; (286; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in SEQ ID NOs: 230 and 460; [G22con-C04con].

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 256 and 461; (286)
2) SEQ ID NOs: 258 and 462; (788)
3) SEQ ID NOs: 257 and 461; (286) or
4) SEQ ID NOs: 259 and 462; (788).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 584-587. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 705-708 (bispecific antibodies 286 and 788; G22-C04 and humanized and consensus versions thereof).

### Bispecific L05-B12

In some embodiments, provided is a bispecific antibody comprising a first single immunoglobulin variable domain (VHH) and a second VHH, wherein the first VHH specifically binds to the extracellular domain of interleukin-10 receptor (IL-10R) alpha subunit (IL 10Rα) and the second VHH specifically binds to the extracellular domain of interleukin-10 receptor beta subunit (IL-10Rβ). In some embodiments, the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, respectively:
1) SEQ ID NOs: 19, 51, 52, 302, 303 and 304; (247; according to Kabat)
2) SEQ ID NOs: 74, 75, 104, 340, 341 and 342; (247; according to IMGT)
3) SEQ ID NOs: 124, 125, 126, 380, 381 and 381 (247; according to Chothia) or
4) SEQ ID NOs: 204, 205, 206, 420, 421 and 422; (247; according to Honegger).

In some embodiments, the bispecific antibody comprises a first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 278 and 456; (247)
2) SEQ ID NOs: 280 and 457; (749)
3) SEQ ID NOs: 279 and 456; (247) or
4) SEQ ID NOs: 280 and 457; (749).

In some embodiments, the bispecific antibody comprises in tandem the first VHH that specifically binds to IL-10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 588-591. In some embodiments, the bispecific antibody comprises at least one polypeptide (e.g., a first polypeptide and a second polypeptide) comprising the following amino acid sequence, or amino acid sequences that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 701-704 (bispecific antibodies 247 and 748; L05-B12 and humanized and consensus versions thereof).

### 5. Polynucleotides

Provided is a polynucleotide or multiple polynucleotides encoding one or more of the anti-IL-10Rα VHH, the anti-IL-10Rβ VHH, the anti-IL-10Rαβ VHH1-VHH2, and the anti-IL-10Rαβ VHH1-VHH2-Fc, described herein, vectors comprising such polynucleotides, and host cells (e.g., mammalian cells, plant cells, yeast cells, bacteria cells including E. coli cells) comprising such polynucleotides or expression vectors. Provided herein are polynucleotides comprising nucleotide sequence(s) encoding any of the anti-IL-10Rα VHH, the anti-IL-10Rβ VHH, the anti-IL-10Rαβ VHH1-VHH2, and the anti-IL-10Rαβ VHH1-VHH2-Fc provided herein, as well as expression cassettes and vector(s) comprising such polynucleotide sequences, e.g., expression vectors for their efficient expression in host cells, e.g., mammalian cells.

The terms “polynucleotide” and “nucleic acid molecule” interchangeably refer to a polymeric form of nucleotides and includes both sense and anti-sense strands of RNA, cDNA, genomic DNA, and synthetic forms and mixed polymers of the above. As used herein, the term nucleic acid molecule may be interchangeable with the term polynucleotide. In some embodiments, a nucleotide refers to a ribonucleotide, deoxynucleotide or a modified form of either type of nucleotide, and combinations thereof. The terms also include, but are not limited to, single- and double-stranded forms of DNA. In addition, a polynucleotide, e.g., a cDNA or mRNA, may include either or both naturally occurring and modified nucleotides linked together by either or both of naturally occurring and non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-biased polynucleotides for improved expression in a desired host cell.

A “substitution,” as used herein, denotes the replacement of one or more amino acids or nucleotides by different amino acids or nucleotides, respectively.

A polynucleotide “variant,” as the term is used herein, is a polynucleotide that typically differs from a polynucleotide specifically disclosed herein in one or more substitutions, deletions, additions and insertions. Such variants may be naturally occurring or may be synthetically generated, for example, by modifying one or more of the polynucleotide sequences described herein and evaluating one or more biological activities of the encoded polypeptide as described herein, e.g., using any of a number of techniques well known in the art.

An “isolated” nucleic acid molecule has been separated from a component of its natural environment. An isolated nucleic acid molecule can be expressed from cells that ordinarily contain the nucleic acid molecule, but the nucleic acid molecule is present extra-chromosomally or at a chromosomal location that is different from its natural chromosomal location.

Further provided are polynucleotides or nucleic acid molecules encoding the anti-IL-10Rα VHH, the anti-IL-10Rβ VHH, the anti-IL-10Rαβ VHH1-VHH2, or the anti-IL-10Rαβ VHH1-VHH2-Fc, as described herein. In some embodiments, the polynucleotides or nucleic acid molecules are DNA, cDNA, or mRNA. In some other embodiments, the polynucleotides or nucleic acid molecules are codon-biased to enhance expression in a desired host cell, e.g., a mammalian host cell, e.g., a Chinese hamster ovary (CHO) cell. In certain instances, the polynucleotide or polynucleotides encode one or more of the IL-10Rα VHH described in Tables A1-A4 and B. In certain instances, the polynucleotide or polynucleotides encode one or more of the anti-IL-10Rβ VHH described in Tables C1-C4 and D. In certain instances, the polynucleotide or polynucleotides encode one or more of the anti-IL-10Rαβ VHH1-VHH2, described in Table E. In certain instances, the polynucleotide or polynucleotides encode one or more of the anti-IL-10Rαβ VHH1-VHH2-Fc, described in Table F.

Also encompassed by this disclosure are polynucleotides encoding the anti-IL-10Rα VHH, the anti-IL-10Rβ VHH, the anti-IL-10Rαβ VHH1-VHH2, and the anti-IL-10Rαβ VHH1-VHH2-Fc described herein, that have at least one of codon-biased sequences for improved expression in a desired host cell, replacement heterologous signal sequences, and reduced or eliminated mRNA instability elements. Methods to generate codon-biased nucleic acids can be carried out by adapting the methods described in, e.g., U.S. Pat. Nos. 5,965,726; 6,174,666; 6,291,664; 6,414,132; and 6,794,498. Preferred codon usage for expression of the anti-IL-10Rα VHH, the anti-IL-10Rβ VHH, the anti-IL-10Rαβ VHH1-VHH2, or the anti-IL-10Rαβ VHH1-VHH2-Fc in desired host cells is provided, e.g., at kazusa.or.jp/codon/; and genscript.com/tools/codon-frequency-table.

As appropriate, in certain embodiments, the 3′-end of the polynucleotides encoding the anti-IL-10Rα VHH, the anti-IL-10Rβ VHH, the anti-IL-10Rαβ VHH1-VHH2, or the anti-IL-10Rαβ VHH1-VHH2-Fc comprises one or multiple tandem stop codons, e.g., two or more tandem TAG (“amber”), TAA (“ochre”) or TGA (“opal” or “umber”) stop codons. The multiple tandem stop codons can be the same or different.

In some embodiments, the one or more polynucleotides (e.g., mRNA) encoding the anti-IL-10Rα VHH, the anti-IL-10Rβ VHH, the anti-IL-10Rαβ VHH1-VHH2, and the anti-IL-10Rαβ VHH1-VHH2-Fc, described herein, are formulated or encapsulated in a lipoplex, e.g., a lipid nanoparticle (LNP). As used herein, a “lipoplex” refers to cationic liposomes that are nonviral (synthetic) lipid carriers of DNA. In some embodiments the lipoplex is a lipid nanoparticle (LNP). As used herein, the term “lipid nanoparticle” refers to one or more spherical nanoparticles with an average diameter of between 10 to 1000 nanometers, and which comprise a solid lipid core matrix that can solubilize lipophilic molecules. In certain embodiments, the lipid core is stabilized by surfactants (e.g., emulsifiers), and can comprise one or more of triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, each of which is incorporated by reference in its entirety. In certain embodiments, the one or more polynucleotides encoding the antibodies or antigen-binding fragments, described herein, are formulated or encapsulated in an LNP comprised of an ionizable cationic lipid/phosphatidylcholine/cholesterol/PEG-lipid, e.g., in molar ratios of about 50:10:38.5:1.5 mol mol⁻¹, respectively.

In some embodiments, the polynucleotide encoding the sdAb that binds to IL-10Rα comprises a polynucleotide of any one of SEQ ID NOs: 764-769, or a polynucleotide that is at least 80%, at least 85%, at least 90%, at least 91%, at least 92%, at least 93%, at least 94%, at least 95%, at least 96%, at least 97%, at least 98%, or at least 99% identical to the full length of a polynucleotide of any one of SEQ ID NOs: 764-769. In some embodiments, the polynucleotide encoding the sdAb that binds to IL-10Rβ comprises a polynucleotide of any one of SEQ ID NOs: 770-775, or a polynucleotide that is at least 80%, at least 85%, at least 90%, at least 91%, at least 92%, at least 93%, at least 94%, at least 95%, at least 96%, at least 97%, at least 98%, or at least 99% identical to the full length of a polynucleotide of any one of SEQ ID NOs: 770-775. In some embodiments, the polynucleotide encoding the bispecific antibody comprises a polynucleotide of any one of SEQ ID NOs: 776-787, or a polynucleotide that is at least 80%, at least 85%, at least 90%, at least 91%, at least 92%, at least 93%, at least 94%, at least 95%, at least 96%, at least 97%, at least 98%, or at least 99% identical to the full length of a polynucleotide of any one of SEQ ID NOs: 776-787. Illustrative polynucleotide sequences are provided in Table G.

TABLE G
Illustrative Polynucleotide sequences

| αIL- | |
|---|---|
| 10Rα | Polynucleotide Sequence |
| huE07 | SEQ ID NO: 764 |
| | gcaagtcaactacaactactagaaagtggtggcggactggtccaacccggcggct |
| | ccctgagactgtcttgcgccgcttctggccggaccttctccgcctacggcatggg |
| | ctggttcagacaggcccctggcaagggcagagagttcgtgagcgccatcagatgg |
| | tacggcgagggcacctactacgccgagtccgtgaaaggcagattcaccatcagca |
| | aggacaactccaaaaacaccctgtacctgcagatgaactccctgcgggctgagga |
| | caccgctgtgtactactgtgctgctaagacccggcctgactcctacctgagcgag |
| | aagtcttatggctattggggccagggcacaatggtcacagtgtcctcc |
| huE07 | SEQ ID NO: 765 |
| | gcaagtcaactacaactactagaaagtggtggaggcctcgtccaacccggcggca |
| | gcctgcggctgagctgtgctgcctctggccggaccttctccgcctacggcatggg |
| | ctggttccggcaggctcctggcaaaggcagagagttcgtgagcgccatcagatgg |
| | atcggcgacgctacctactacgctgagtccgtgaagggccggttcaccatctcca |
| | aggacaactccaagaacaccctgtacctgcagatgaattctctgcgcgccgagga |
| | caccgccgtgtactactgtgccgccaagacctctgttacctcctacctgtctgag |
| | aagtcttacaactactggggccagggcaccatggtgacagtgtcctct |
| huE07 | SEQ ID NO: 766 |
| | gcctctcagctccagctgttggaatctggcggaggattggttcagcctggcggct |
| | ctctgagactgtcttgtgctgcttccggcagaaccttcagcgcttatggcatggg |
| | ctggttcagacaggctcctggcaagggcagagaattcgtgtccgccatcagatgg |
| | atcggcgacgccacctactacgccgagtctgtgaagggcagattcaccatctcca |
| | aggacaactccaagaacaccctgtacctgcagatgaactccctgagagccgagga |
| | caccgccgtgtactactgtgccgctaagacctccgtgacctcctacctgtccgag |
| | aagtcctacaactactggggccagggcaccatggtcaccgtttctagc |
| huF11 | SEQ ID NO: 767 |
| | gaagtgcagctactggagagcggcggtggcctggtccagccaggagggagcttga |
| | gattgagttgcgctgcctcaggtcgaacattcagttctcacaccgtgggatggtt |
| | ccggcaagcccctggtaagggaagagagttcgtcgctgccataacttggaacggg |
| | gataccacatacttcggggattttgtcgagggaaggttcacaatttctagggata |
| | actccaagaacacactgtatcttcagatgaactctctccgggctgaagataccgc |
| | tgtatactattgcgctgcagccactggtcaagtgtggagcctccagtatcttcga |
| | gaaagtagctacgcttactggggccagggcaccctggtcacagtgtcttcc |
| huF11 | SEQ ID NO: 768 |
| | gagctacagctgctcgagtctggaggcgggctggtacagcccggtgggagcctca |
| | ggctgagctgcgctgcaagcgggagaacgttcagcagtcatactgtgggctggtt |
| | caggcaggcacccgggaagggtcgagaattcgtgtccgctatcacctggaatggg |
| | gacacaacctactttggtgatttcgtggaggggcgattcacaatctcaagggata |
| | actccaagaacaccctgtacctacagatgaactcccttagggcagaagatacagc |
| | tgtgtactattgcgctgcagcgaccggtcaggtgtggtcactccagtacctgaga |
| | gaatccagttatgcctactggggtcagggcaccctagtcacagtgtcttcc |
| huF11 | SEQ ID NO: 769 |
| | gaactgcagctgttggaatctggcggcggattggttcagcctggcggatctctga |
| | gactgtcttgtgccgcttccggcagaaccttctcctctcataccgtcggctggtt |
| | cagacaggctcctggcaagggcagagaattcgtgtctgccatcacctggaacggc |
| | gacaccacctacttcggcgatttcgtggaaggccggttcaccatctctcgggaca |
| | actccaagaacaccctgtacctgcagatgaactccctgagagccgaggacaccgc |
| | cgtgtactattgtgctgctgctaccggccaagtgtggtccctgcagtatctgaga |
| | gagtcctcctacgcctattggggccagggcacactggtcacagtttctagt |
| αIL- | |
| 10Rβ | Polynucleotide Sequence |
| huA05 | SEQ ID NO: 770 |
| | gaagtgcagctggtggaatccggcggaggcctcgtgcagcctggaggctctctgc |
| | ggctgtcctgcgccgcctctggcaactccctggactaccctaccatcggctggtt |
| | ccggcaggctccaggcaagggcctggaaggcgtgtctgctatctctgcctccgac |
| | gcctctacccactacgccgacgctgtgaagggaaggtttacaatctccagagata |
| | attctaagaacaccgtgtacctgcagatgaactctctgagagccgaggataccgc |
| | cgtgtactactgcgctgctagcctgcacggcctgggcgccaagtccgaggccgat |
| | tacgactactggggccagggcaccatggtgaccgtgtccagc |
| huA05 | SEQ ID NO: 771 |
| | gaagtgcagctggtggaatctggcggcggactggtgcagcctggaggatctctga |
| | gactgtcctgcgctgcttccggcaactccctggactactccacaatcggctggtt |
| | tagacaggcccctggcaagggcctggaaggcgtgtcttgcatctctgcctccgag |
| | ggctctacccactacgccgacgccgtgaaaggcagattcaccatcagcagagaca |
| | acagcaagaacaccgtgtatctgcagatgaactccctgagagctgaggatacagc |
| | tgtgtactattgcgccgcttctctgcacggcctgggctgcaagtccgaggccgat |
| | tacgattactggggacagggcaccatggtcaccgtgtcctcc |
| huA05 | SEQ ID NO: 772 |
| | gaggtgcagctggttgaaagtggtggcggacttgtgcaacctggcggaagtctga |
| | gactcagctgtgccgcctctggcaactccctggactattctaccatcggctggtt |
| | taggcaggcacccggaaaaggactggaaggcgtgtcctgtatctccgcctctgag |
| | ggctctacccactacgctgatgccgtgaaaggccggttcaccatcagccgggaca |
| | acagcaagaataccgtctatctccagatgaacagcctgcgcgctgaggatacagc |
| | tgtgtactattgcgccgcctctctgcatggcctgggctgcaaatctgaggccgac |
| | tacgattattggggacagggaacaatggttacagtgtcctcc |
| huC04 | SEQ ID NO: 773 |
| | gaggtgcagctggtggagtctggaggtggcttggtgcagccgggcggatccctac |
| | gcctctcatgtagcgcttccggatttacctccccttatagcaccatagggtggtt |
| | ccggcaggctccagggaaaggacgcgaaggggtggcctgtatcagctcttccggc |
| | cgaggcgtgaatgcgaattacatcgactcagtgaggggtcgcttcaccatcagcc |
| | gggacaacagcaagaacacagtttacctccaaatgaacagcctgagggccgagga |
| | cacagctgtgtattactgtgctacaaactgctggcgccttacaagtcccgcagcc |
| | gataattggggtcagggaactatggtcacagtgtcttcc |
| huC04 | SEQ ID NO: 774 |
| | gaggtgcagctggtggagtctggaggtggcttggtgcagccgggcggatccctac |
| | gcctctcatgtagcgcttccggatttacctccccttatagcaccatagggtggtt |
| | ccggcaggctccagggaaaggacgcgaaggggtggcctgtatcagctcttccggc |
| | cgaggcgtgaatgcgaattacatcgactcagtgaggggtcgcttcaccatcagcc |
| | gggacaacagcaagaacacagtttacctccaaatgaacagcctgagggccgagga |
| | cacagctgtgtattactgtgctacaaactgctggcgccttacaagtcccgcagcc |
| | gataattggggtcagggaactatggtcacagtgtcttcc |
| huC04 | SEQ ID NO: 775 |
| | gaggtgcagctggttgaaagcggcggaggacttgttcaaccaggcggaagcctgc |
| | ggctgtcttgttctgcctctggcttcacctctccttactccaccatcggatggtt |
| | ccggcaggcccctggaaagggacgagaaggtgtcgcctgcatctccagctctgga |
| | agaggcgtgaacgccaactacatcgactctgtgcggggcagattcaccatcagca |
| | gagacaacagcaagaataccgtgtatctccaaatgaacagcctgcgcgctgagga |
| | taccgctgtgtattactgcgccaccaactgctggcggctgacctctcctgctgct |
| | gataattggggacagggaactatggttaccgtgtcctcc |
| VHH1- | |
| VHH2 | Polynucleotide Sequence |
| 647 | SEQ ID NO: 776 |
| | gcaagtcaactacaactactagaaagtggtggcggactggtccaacccggcggct |
| | ccctgagactgtcttgcgccgcttctggccggaccttctccgcctacggcatggg |
| | ctggttcagacaggcccctggcaagggcagagagttcgtgagcgccatcagatgg |
| | tacggcgagggcacctactacgccgagtccgtgaaaggcagattcaccatcagca |
| | aggacaactccaaaaacaccctgtacctgcagatgaactccctgcgggctgagga |
| | caccgctgtgtactactgtgctgctaagacccggcctgactcctacctgagcgag |
| | aagtcttatggctattggggccagggcacaatggtcacagtgtcctccggcggag |
| | gatctgaagtgcagctggtggaatccggcggaggcctcgtgcagcctggaggctc |
| | tctgcggctgtcctgcgccgcctctggcaactccctggactaccctaccatcggc |
| | tggttccggcaggctccaggcaagggcctggaaggcgtgtctgctatctctgcct |
| | ccgacgcctctacccactacgccgacgctgtgaagggaaggtttacaatctccag |
| | agataattctaagaacaccgtgtacctgcagatgaactctctgagagccgaggat |
| | accgccgtgtactactgcgctgctagcctgcacggcctgggcgccaagtccgagg |
| | ccgattacgactactggggccagggcaccatggtgaccgtgtccagc |
| 195 | SEQ ID NO: 777 |
| | gcaagtcaactacaactactagaaagtggtggaggcctcgtccaacccggcggca |
| | gcctgcggctgagctgtgctgcctctggccggaccttctccgcctacggcatggg |
| | ctggttccggcaggctcctggcaaaggcagagagttcgtgagcgccatcagatgg |
| | atcggcgacgctacctactacgctgagtccgtgaagggccggttcaccatctcca |
| | aggacaactccaagaacaccctgtacctgcagatgaattctctgcgcgccgagga |
| | caccgccgtgtactactgtgccgccaagacctctgttacctcctacctgtctgag |
| | aagtcttacaactactggggccagggcaccatggtgacagtgtcctctggtggcg |
| | gcagcgaagtgcagctggtggaatctggcggcggactggtgcagcctggaggatc |
| | tctgagactgtcctgcgctgcttccggcaactccctggactactccacaatcggc |
| | tggtttagacaggcccctggcaagggcctggaaggcgtgtcttgcatctctgcct |
| | ccgagggctctacccactacgccgacgccgtgaaaggcagattcaccatcagcag |
| | agacaacagcaagaacaccgtgtatctgcagatgaactccctgagagctgaggat |
| | acagctgtgtactattgcgccgcttctctgcacggcctgggctgcaagtccgagg |
| | ccgattacgattactggggacagggcaccatggtcaccgtgtcctcc |
| 195 | SEQ ID NO: 778 |
| | gcctctcagctccagctgttggaatctggcggaggattggttcagcctggcggct |
| | ctctgagactgtcttgtgctgcttccggcagaaccttcagcgcttatggcatggg |
| | ctggttcagacaggctcctggcaagggcagagaattcgtgtccgccatcagatgg |
| | atcggcgacgccacctactacgccgagtctgtgaagggcagattcaccatctcca |
| | aggacaactccaagaacaccctgtacctgcagatgaactccctgagagccgagga |
| | caccgccgtgtactactgtgccgctaagacctccgtgacctcctacctgtccgag |
| | aagtcctacaactactggggccagggcaccatggtcaccgtttctagcggaggcg |
| | gatctgaggtgcagctggttgaaagtggtggcggacttgtgcaacctggcggaag |
| | tctgagactcagctgtgccgcctctggcaactccctggactattctaccatcggc |
| | tggtttaggcaggcacccggaaaaggactggaaggcgtgtcctgtatctccgcct |
| | ctgagggctctacccactacgctgatgccgtgaaaggccggttcaccatcagccg |
| | ggacaacagcaagaataccgtctatctccagatgaacagcctgcgcgctgaggat |
| | acagctgtgtactattgcgccgcctctctgcatggcctgggctgcaaatctgagg |
| | ccgactacgattattggggacagggaacaatggttacagtgtcctcc |
| 183 | SEQ ID NO: 779 |
| | gaagtgcagctactggagagcggcggtggcctggtccagccaggagggagcttga |
| | gattgagttgcgctgcctcaggtcgaacattcagttctcacaccgtgggatggtt |
| | ccggcaagcccctggtaagggaagagagttcgtcgctgccataacttggaacggg |
| | gataccacatacttcggggattttgtcgagggaaggttcacaatttctagggata |
| | actccaagaacacactgtatcttcagatgaactctctccgggctgaagataccgc |
| | tgtatactattgcgctgcagccactggtcaagtgtggagcctccagtatcttcga |
| | gaaagtagctacgcttactggggccagggcaccctggtcacagtgtcttccggag |
| | gcggaagcgaggtgcagctggtggagtctggaggtggcttggtgcagccgggcgg |
| | atccctacgcctctcatgtagcgcttccggatttacctccccttatagcaccata |
| | gggtggttccggcaggctccagggaaaggacgcgaaggggtggcctgtatcagct |
| | cttccggccgaggcgtgaatgcgaattacatcgactcagtgaggggtcgcttcac |
| | catcagccgggacaacagcaagaacacagtttacctccaaatgaacagcctgagg |
| | gccgaggacacagctgtgtattactgtgctacaaactgctggcgccttacaagtc |
| | ccgcagccgataattggggtcagggaactatggtcacagtgtcttcc |
| 184 | SEQ ID NO: 780 |
| | gagctacagctgctcgagtctggaggcgggctggtacagcccggtgggagcctca |
| | ggctgagctgcgctgcaagcgggagaacgttcagcagtcatactgtgggctggtt |
| | caggcaggcacccgggaagggtcgagaattcgtgtccgctatcacctggaatggg |
| | gacacaacctactttggtgatttcgtggaggggcgattcacaatctcaagggata |
| | actccaagaacaccctgtacctacagatgaactcccttagggcagaagatacagc |
| | tgtgtactattgcgctgcagcgaccggtcaggtgtggtcactccagtacctgaga |
| | gaatccagttatgcctactggggtcagggcaccctagtcacagtgtcttccggag |
| | gcggaagcgaggtgcagctggtggagtctggaggtggcttggtgcagccgggcgg |
| | atccctacgcctctcatgtagcgcttccggatttacctccccttatagcaccata |
| | gggtggttccggcaggctccagggaaaggacgcgaaggggtggcctgtatcagct |
| | cttccggccgaggcgtgaatgcgaattacatcgactcagtgaggggtcgcttcac |
| | catcagccgggacaacagcaagaacacagtttacctccaaatgaacagcctgagg |
| | gccgaggacacagctgtgtattactgtgctacaaactgctggcgccttacaagtc |
| | ccgcagccgataattggggtcagggaactatggtcacagtgtcttcc |
| 184 | SEQ ID NO: 781 |
| | gaactgcagctgttggaatctggcggcggattggttcagcctggcggatctctga |
| | gactgtcttgtgccgcttccggcagaaccttctcctctcataccgtcggctggtt |
| | cagacaggctcctggcaagggcagagaattcgtgtctgccatcacctggaacggc |
| | gacaccacctacttcggcgatttcgtggaaggccggttcaccatctctcgggaca |
| | actccaagaacaccctgtacctgcagatgaactccctgagagccgaggacaccgc |
| | cgtgtactattgtgctgctgctaccggccaagtgtggtccctgcagtatctgaga |
| | gagtcctcctacgcctattggggccagggcacactggtcacagtttctagtggcg |
| | gaggctccgaggtgcagctggttgaaagcggcggaggacttgttcaaccaggcgg |
| | aagcctgcggctgtcttgttctgcctctggcttcacctctccttactccaccatc |
| | ggatggttccggcaggcccctggaaagggacgagaaggtgtcgcctgcatctcca |
| | gctctggaagaggcgtgaacgccaactacatcgactctgtgcggggcagattcac |
| | catcagcagagacaacagcaagaataccgtgtatctccaaatgaacagcctgcgc |
| | gctgaggataccgctgtgtattactgcgccaccaactgctggcggctgacctctc |
| | ctgctgctgataattggggacagggaactatggttaccgtgtcctcc |
| VHH1- | |
| VHH2-Fc | Polynucleotide Sequence |
| 647 | SEQ ID NO: 782 |
| | gcaagtcaactacaactactagaaagtggtggcggactggtccaacccggcggct |
| | ccctgagactgtcttgcgccgcttctggccggaccttctccgcctacggcatggg |
| | ctggttcagacaggcccctggcaagggcagagagttcgtgagcgccatcagatgg |
| | tacggcgagggcacctactacgccgagtccgtgaaaggcagattcaccatcagca |
| | aggacaactccaaaaacaccctgtacctgcagatgaactccctgcgggctgagga |
| | caccgctgtgtactactgtgctgctaagacccggcctgactcctacctgagcgag |
| | aagtcttatggctattggggccagggcacaatggtcacagtgtcctccggcggag |
| | gatctgaagtgcagctggtggaatccggcggaggcctcgtgcagcctggaggctc |
| | tctgcggctgtcctgcgccgcctctggcaactccctggactaccctaccatcggc |
| | tggttccggcaggctccaggcaagggcctggaaggcgtgtctgctatctctgcct |
| | ccgacgcctctacccactacgccgacgctgtgaagggaaggtttacaatctccag |
| | agataattctaagaacaccgtgtacctgcagatgaactctctgagagccgaggat |
| | accgccgtgtactactgcgctgctagcctgcacggcctgggcgccaagtccgagg |
| | ccgattacgactactggggccagggcaccatggtgaccgtgtccagcggttctga |
| | caagacccacacctgtcctccatgtcctgctccagaagctgctggcgccccatcc |
| | gtgtttctgttccctccaaagcctaaggacaccctgatgatctctcggacccctg |
| | aagtgacctgcgtggtggtggatgtgtctcacgaggatcccgaagtgaagttcaa |
| | ttggtacgtggacggcgtggaagtgcacaacgccaagaccaagcctagagaggaa |
| | cagtacaactccacctacagagtggtgtccgtgctgaccgtgctgcaccaggatt |
| | ggctgaacggcaaagagtacaagtgcaaggtgtccaacaaggccctgcctgctcc |
| | tatcgaaaagaccatctccaaggccaagggccagcctagggaaccccaggtttac |
| | accctgcctccaagccgggaagagatgaccaagaaccaggtgtccctgacctgcc |
| | tcgtgaagggcttctacccttccgatatcgccgtggaatgggagagcaatggcca |
| | gcctgagaacaactacaagacaacccctcctgtgctggactccgacggctcattc |
| | ttcctgtactccaagctgacagtggacaagtccagatggcagcagggcaacgtgt |
| | tctcctgctccgtgatgcacgaggccctgcacaatcactacacccagaagtccct |
| | gtctctgtcccctggaaaa |
| 195 | SEQ ID NO: 783 |
| | gcaagtcaactacaactactagaaagtggtggaggcctcgtccaacccggcggca |
| | gcctgcggctgagctgtgctgcctctggccggaccttctccgcctacggcatggg |
| | ctggttccggcaggctcctggcaaaggcagagagttcgtgagcgccatcagatgg |
| | atcggcgacgctacctactacgctgagtccgtgaagggccggttcaccatctcca |
| | aggacaactccaagaacaccctgtacctgcagatgaattctctgcgcgccgagga |
| | caccgccgtgtactactgtgccgccaagacctctgttacctcctacctgtctgag |
| | aagtcttacaactactggggccagggcaccatggtgacagtgtcctctggtggcg |
| | gcagcgaagtgcagctggtggaatctggcggcggactggtgcagcctggaggatc |
| | tctgagactgtcctgcgctgcttccggcaactccctggactactccacaatcggc |
| | tggtttagacaggcccctggcaagggcctggaaggcgtgtcttgcatctctgcct |
| | ccgagggctctacccactacgccgacgccgtgaaaggcagattcaccatcagcag |
| | agacaacagcaagaacaccgtgtatctgcagatgaactccctgagagctgaggat |
| | acagctgtgtactattgcgccgcttctctgcacggcctgggctgcaagtccgagg |
| | ccgattacgattactggggacagggcaccatggtcaccgtgtcctccggctccga |
| | caagacccacacctgtcctccatgtcctgctccagaagctgctggcgccccatcc |
| | gtgtttctgttccctccaaagcctaaggacaccctgatgatctctcggacccctg |
| | aagtgacctgcgtggtggtggatgtgtctcacgaggatcccgaagtgaagttcaa |
| | ttggtacgtggacggcgtggaagtgcacaacgccaagaccaagcctagagaggaa |
| | cagtacaactccacctacagagtggtgtccgtgctgaccgtgctgcaccaggatt |
| | ggctgaacggcaaagagtacaagtgcaaggtgtccaacaaggccctgcctgctcc |
| | tatcgaaaagaccatctccaaggccaagggccagcctagggaaccccaggtttac |
| | accctgcctccaagccgggaagagatgaccaagaaccaggtgtccctgacctgcc |
| | tcgtgaagggcttctacccttccgatatcgccgtggaatgggagagcaatggcca |
| | gcctgagaacaactacaagacaacccctcctgtgctggactccgacggctcattc |
| | ttcctgtactccaagctgacagtggacaagtccagatggcagcagggcaacgtgt |
| | tctcctgctccgtgatgcacgaggccctgcacaatcactacacccagaagtccct |
| | gtctctgtcccctggaaaa |
| 195 | SEQ ID NO: 784 |
| | gcctctcagctccagctgttggaatctggcggaggattggttcagcctggcggct |
| | ctctgagactgtcttgtgctgcttccggcagaaccttcagcgcttatggcatggg |
| | ctggttcagacaggctcctggcaagggcagagaattcgtgtccgccatcagatgg |
| | atcggcgacgccacctactacgccgagtctgtgaagggcagattcaccatctcca |
| | aggacaactccaagaacaccctgtacctgcagatgaactccctgagagccgagga |
| | caccgccgtgtactactgtgccgctaagacctccgtgacctcctacctgtccgag |
| | aagtcctacaactactggggccagggcaccatggtcaccgtttctagcggaggcg |
| | gatctgaggtgcagctggttgaaagtggtggcggacttgtgcaacctggcggaag |
| | tctgagactcagctgtgccgcctctggcaactccctggactattctaccatcggc |
| | tggtttaggcaggcacccggaaaaggactggaaggcgtgtcctgtatctccgcct |
| | ctgagggctctacccactacgctgatgccgtgaaaggccggttcaccatcagccg |
| | ggacaacagcaagaataccgtctatctccagatgaacagcctgcgcgctgaggat |
| | acagctgtgtactattgcgccgcctctctgcatggcctgggctgcaaatctgagg |
| | ccgactacgattattggggacagggaacaatggttacagtgtcctccggcagcga |
| | caagacccacacctgtcctccatgtcctgctccagaagctgctggcgccccttcc |
| | gtgtttctgttccctccaaagcctaaggacaccctgatgatctctcggacccctg |
| | aagtgacctgcgtggtggtggatgtgtctcacgaggatcccgaagtgaagttcaa |
| | ttggtacgtggacggcgtggaagtgcacaacgccaagaccaagcctagagaggaa |
| | cagtacaactccacctacagagtggtgtccgtgctgaccgtgctgcaccaggatt |
| | ggctgaacggcaaagagtacaagtgcaaggtgtccaacaaggccctgcctgctcc |
| | tatcgaaaagaccatcagcaaggccaagggccagcctagggaaccccaggtttac |
| | accctgcctccaagccgggaagagatgaccaagaaccaggtgtccctgacctgcc |
| | tggtcaagggcttctacccttccgatatcgccgtggaatgggagagcaatggcca |
| | gcctgagaacaactacaagacaacccctcctgtgctggactccgacggctcattc |
| | ttcctgtactccaagctgacagtggacaagtccagatggcagcagggcaacgtgt |
| | tctcctgctccgtgatgcacgaggccctgcacaatcactacacccagaagtccct |
| | gtctctgtcccctggcaag |
| 183 | SEQ ID NO: 785 |
| | gaagtgcagctactggagagcggcggtggcctggtccagccaggagggagcttga |
| | gattgagttgcgctgcctcaggtcgaacattcagttctcacaccgtgggatggtt |
| | ccggcaagcccctggtaagggaagagagttcgtcgctgccataacttggaacggg |
| | gataccacatacttcggggattttgtcgagggaaggttcacaatttctagggata |
| | actccaagaacacactgtatcttcagatgaactctctccgggctgaagataccgc |
| | tgtatactattgcgctgcagccactggtcaagtgtggagcctccagtatcttcga |
| | gaaagtagctacgcttactggggccagggcaccctggtcacagtgtcttccggag |
| | gcggaagcgaggtgcagctggtggagtctggaggtggcttggtgcagccgggcgg |
| | atccctacgcctctcatgtagcgcttccggatttacctccccttatagcaccata |
| | gggtggttccggcaggctccagggaaaggacgcgaaggggtggcctgtatcagct |
| | cttccggccgaggcgtgaatgcgaattacatcgactcagtgaggggtcgcttcac |
| | catcagccgggacaacagcaagaacacagtttacctccaaatgaacagcctgagg |
| | gccgaggacacagctgtgtattactgtgctacaaactgctggcgccttacaagtc |
| | ccgcagccgataattggggtcagggaactatggtcacagtgtcttccggatcaga |
| | caagacccacacctgtcctccatgtcctgctccagaagctgctggcgccccatcc |
| | gtgtttctgttccctccaaagcctaaggacaccctgatgatctctcggacccctg |
| | aagtgacctgcgtggtggtggatgtgtctcacgaggatcccgaagtgaagttcaa |
| | ttggtacgtggacggcgtggaagtgcacaacgccaagaccaagcctagagaggaa |
| | cagtacaactccacctacagagtggtgtccgtgctgaccgtgctgcaccaggatt |
| | ggctgaacggcaaagagtacaagtgcaaggtgtccaacaaggccctgcctgctcc |
| | tatcgaaaagaccatctccaaggccaagggccagcctagggaaccccaggtttac |
| | accctgcctccaagccgggaagagatgaccaagaaccaggtgtccctgacctgcc |
| | tcgtgaagggcttctacccttccgatatcgccgtggaatgggagagcaatggcca |
| | gcctgagaacaactacaagacaacccctcctgtgctggactccgacggctcattc |
| | ttcctgtactccaagctgacagtggacaagtccagatggcagcagggcaacgtgt |
| | tctcctgctccgtgatgcacgaggccctgcacaatcactacacccagaagtccct |
| | gtctctgtcccctggaaaa |
| | SEQ ID NO: 786 |
| 184 | gagctacagctgctcgagtctggaggcgggctggtacagcccggtgggagcctca |
| | ggctgagctgcgctgcaagcgggagaacgttcagcagtcatactgtgggctggtt |
| | caggcaggcacccgggaagggtcgagaattcgtgtccgctatcacctggaatggg |
| | gacacaacctactttggtgatttcgtggaggggcgattcacaatctcaagggata |
| | actccaagaacaccctgtacctacagatgaactcccttagggcagaagatacagc |
| | tgtgtactattgcgctgcagcgaccggtcaggtgtggtcactccagtacctgaga |
| | gaatccagttatgcctactggggtcagggcaccctagtcacagtgtcttccggag |
| | gcggaagcgaggtgcagctggtggagtctggaggtggcttggtgcagccgggcgg |
| | atccctacgcctctcatgtagcgcttccggatttacctccccttatagcaccata |
| | gggtggttccggcaggctccagggaaaggacgcgaaggggtggcctgtatcagct |
| | cttccggccgaggcgtgaatgcgaattacatcgactcagtgaggggtcgcttcac |
| | catcagccgggacaacagcaagaacacagtttacctccaaatgaacagcctgagg |
| | gccgaggacacagctgtgtattactgtgctacaaactgctggcgccttacaagtc |
| | ccgcagccgataattggggtcagggaactatggtcacagtgtcttccggatcaga |
| | caagacccacacctgtcctccatgtcctgctccagaagctgctggcgccccatcc |
| | gtgtttctgttccctccaaagcctaaggacaccctgatgatctctcggacccctg |
| | aagtgacctgcgtggtggtggatgtgtctcacgaggatcccgaagtgaagttcaa |
| | ttggtacgtggacggcgtggaagtgcacaacgccaagaccaagcctagagaggaa |
| | cagtacaactccacctacagagtggtgtccgtgctgaccgtgctgcaccaggatt |
| | ggctgaacggcaaagagtacaagtgcaaggtgtccaacaaggccctgcctgctcc |
| | tatcgaaaagaccatctccaaggccaagggccagcctagggaaccccaggtttac |
| | accctgcctccaagccgggaagagatgaccaagaaccaggtgtccctgacctgcc |
| | tcgtgaagggcttctacccttccgatatcgccgtggaatgggagagcaatggcca |
| | gcctgagaacaactacaagacaacccctcctgtgctggactccgacggctcattc |
| | ttcctgtactccaagctgacagtggacaagtccagatggcagcagggcaacgtgt |
| | tctcctgctccgtgatgcacgaggccctgcacaatcactacacccagaagtccct |
| | gtctctgtcccctggaaaa |
| 184 | SEQ ID NO: 787 |
| | gaactgcagctgttggaatctggcggcggattggttcagcctggcggatctctga |
| | gactgtcttgtgccgcttccggcagaaccttctcctctcataccgtcggctggtt |
| | cagacaggctcctggcaagggcagagaattcgtgtctgccatcacctggaacggc |
| | gacaccacctacttcggcgatttcgtggaaggccggttcaccatctctcgggaca |
| | actccaagaacaccctgtacctgcagatgaactccctgagagccgaggacaccgc |
| | cgtgtactattgtgctgctgctaccggccaagtgtggtccctgcagtatctgaga |
| | gagtcctcctacgcctattggggccagggcacactggtcacagtttctagtggcg |
| | gaggctccgaggtgcagctggttgaaagcggcggaggacttgttcaaccaggcgg |
| | aagcctgcggctgtcttgttctgcctctggcttcacctctccttactccaccatc |
| | ggatggttccggcaggcccctggaaagggacgagaaggtgtcgcctgcatctcca |
| | gctctggaagaggcgtgaacgccaactacatcgactctgtgcggggcagattcac |
| | catcagcagagacaacagcaagaataccgtgtatctccaaatgaacagcctgcgc |
| | gctgaggataccgctgtgtattactgcgccaccaactgctggcggctgacctctc |
| | ctgctgctgataattggggacagggaactatggttaccgtgtcctccggcagcga |
| | caagacccatacctgtcctccatgtcctgctccagaagctgctggcgccccttcc |
| | gtgtttctgttccctccaaagcctaaggacaccctgatgatctctcggacccctg |
| | aagtgacctgcgtggtggtggatgtgtctcacgaggatcccgaagtgaagttcaa |
| | ttggtacgtggacggcgtggaagtgcacaacgccaagaccaagcctagagaggaa |
| | cagtacaactccacctacagagtggtgtccgtgctgaccgtgctgcaccaggatt |
| | ggctgaacggcaaagagtacaagtgcaaggtgtccaacaaggccctgcctgctcc |
| | tatcgaaaagaccatctccaaggccaagggccagcctagggaaccccaggtttac |
| | accctgcctccaagccgggaagagatgaccaagaaccaggtgtccctgacctgcc |
| | tggtcaagggcttctacccttccgatatcgccgtggaatgggagagcaatggcca |
| | gcctgagaacaactacaagaccacacctcctgtgctggactccgacggctcattc |
| | ttcctgtactccaagctgacagtggacaagtccagatggcagcagggcaacgtgt |
| | tctcctgctccgtgatgcacgaggccctgcacaatcactacacccagaagtccct |
| | gtctctgtcccctggcaag |

### 6. Vectors

Further provided are vectors comprising one or more polynucleotides encoding the anti-IL-10Rα VHH, the anti-IL-10Rβ VHH, the anti-IL-10Rαβ VHH1-VHH2, or the anti-IL-10Rαβ VHH1-VHH2-Fc, as described herein. Also provided are vectors comprising one or more expression cassettes (e.g., multi-cistronic, bicistronic) for expressing one or more of the anti-IL-10Rα VHH, the anti-IL-10Rβ VHH, the anti-IL-10Rαβ VHH1-VHH2, or the anti-IL-10Rαβ VHH1-VHH2-Fc, described herein. Moreover, expression cassettes for expressing polypeptide chains of a biologic molecule comprised of two or more polypeptide chains, e.g., two, three or four polypeptide chains, can be expressed from one or multiple expression vectors, e.g., one, two, three or four vectors. A vector can be of any type, for example, a recombinant vector such as an expression vector. Vectors include, but are not limited to, plasmids, cosmids, bacterial artificial chromosomes (BAC) and yeast artificial chromosomes (YAC) and vectors derived from bacteriophages or plant or animal (including human) viruses. Vectors can comprise an origin of replication and one or more selection markers recognized by and operational in the proposed host cell (e.g., including prokaryotic and eukaryotic host cells), and in the case of expression vectors, promoter, terminator, and other regulatory regions recognized by the host cell. In additional embodiments, a vector comprises an expression cassette as described herein, e.g., comprising a polynucleotide encoding the anti-IL-10Rα VHH, the anti-IL-10Rβ VHH, the anti-IL-10Rαβ VHH1-VHH2, or the anti-IL-10Rαβ VHH1-VHH2-Fc of the disclosure operably linked to a promoter and optionally additional regulatory elements. Certain vectors are capable of autonomous replication in a host into which they are introduced (e.g., vectors having a bacterial origin of replication can replicate in bacteria). Other vectors can be integrated into the genome of a host upon introduction into the host, and thereby are replicated along with the host genome. Vectors include, but are not limited to, those suitable for recombinant production of the antibodies disclosed herein. The term “vector,” as used herein, refers to a nucleic acid molecule capable of propagating another nucleic acid. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule or polynucleotide of the present application. Certain vectors are capable of directing the expression of nucleic acids to which they are operatively linked. Such vectors are referred to herein as expression vectors.

The term “operably linked” refers to two or more nucleic acid sequence elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The choice of the vector is dependent on the recombinant procedures followed and the host used. Introduction of vectors into host cells can be effected by inter alia calcium phosphate transfection, virus infection, DEAE-dextran-mediated transfection, lipofectamine transfection or electroporation. Vectors may be autonomously replicating or may replicate together with the chromosome into which they have been integrated. In certain embodiments, the vectors contain one or more selection markers. The choice of the markers may depend on the host cells of choice. These include, but are not limited to, kanamycin, neomycin, puromycin, hygromycin, zeocin, ampicillin (AmpR), thymidine kinase gene from Herpes simplex virus (HSV-TK), glutamine synthetase (GS) and dihydrofolate reductase (DHFR) gene. Vectors comprising one or more polynucleotides encoding the antibodies described herein, operably linked to one or more polynucleotides encoding proteins or peptides that can be used to isolate the polypeptides of interest, are also covered by the disclosure. These proteins or peptides include, but are not limited to, glutathione-S-transferase, maltose binding protein, metal-binding polyhistidine, green fluorescent protein, luciferase and beta-galactosidase. In other embodiments, the vector that is used is or is based on pcDNA™3.1+(ThermoFisher, MA), pCI (Promega, WI), pIRES (Takara, CA), or pCGS3 (Sigma-Aldrich, MO).

### 7. Host Cells

The disclosure also provides host cells comprising one or more recombinant polynucleotides, one or more expression cassettes, or one or more vectors, as described herein. Any of a variety of host cells can be used, including prokaryotic cells (e.g., bacterial cells, e.g., E. coli cells) and eukaryotic cells. In one embodiment, a host cell is a eukaryotic cell, for example, a yeast cell, a plant cell, an insect cell, a mammalian cell. In some embodiments, the host cell is a mammalian cell such as a Chinese Hamster Ovary (CHO)-based or CHO-originated cell (e.g., CHO-S, CHO DG44, CHO DXB-11, ExpiCHO™, CHOZN® ZFN-modified GS−/− CHO cell line, CHOSOURCE™ GS knockout cell line, CHOK1SV, CHOK1SV GS-KO® cell line, CHO-K1, or other CHO-K1-derived cell lines), a COS cell, a BHK cell, a NS0 cell or a human cell. Examples of human host cells of use include without limitation, inter alia, HeLa, 911, AT1080, A549, HEK293, Expi293™ and HEK293T-cells.

The terms “host cell,” “host cell line,” and “host cell culture” are used interchangeably and refer to cells into which exogenous nucleic acid could be or has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding the anti-IL-10Rα VHH, the anti-IL-10Rβ VHH, the anti-IL-10Rαβ VHH1-VHH2, or the anti-IL-10Rαβ VHH1-VHH2-Fc, as described herein.

If the anti-IL-10Rα VHH, the anti-IL-10Rβ VHH, the anti-IL-10Rα VHH1-VHH2, or the anti-IL-10Rαβ VHH1-VHH2-Fc, as described herein are to be expressed in bacterial cells (e.g., E. coli), the expression vector should have characteristics that permit amplification of the vector in the bacterial cells. Additionally, when E. coli such as JM109, DH5u, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for secretion of the anti-IL-10Rα VHH, the anti-IL-10Rβ VHH, the anti-IL-10Rαβ VHH1-VHH2, or the anti-IL-10Rαβ VHH1-VHH2-Fc molecules. For production into the periplasm of E. coli, the pelB signal sequence (Lei et al., J. Bacteriol., 169: 4379 (1987)) may be used as the signal sequence for secretion of the anti-IL-10Rα VHH, the anti-IL-10Rβ VHH, the anti-IL-10Rαβ VHH1-VHH2, or the anti-IL-10Rαβ VHH1-VHH2-Fc molecules. For bacterial expression, calcium chloride methods or electroporation methods may be used to introduce the expression vector into the bacterial cell.

### 8. Methods of Expression and Purification

The herein described anti-IL-10Rα VHH, anti-IL-10Rβ VHH, anti-IL-10Rα VHH1-VHH2, and anti-IL-10Rαβ VHH1-VHH2-Fc molecules can be produced by any method known in the art for the synthesis of polypeptides, fusion proteins and bispecific molecules, for example, by chemical synthesis or by recombinant expression techniques.

Methods of making monospecific antibodies are known. Methods of making bispecific antibodies are known and described, for example, in PCT Publ. Nos. WO2011/038290; WO2012/158818, WO2012/162067, WO2015/104346, WO2016/086189, WO2016/182751, WO2017/009442, WO2017/125897, WO2017/136659, WO2017/157305, WO2017/201493, WO2018/183139, WO2018/191438, WO2019/034580, WO2019/078697 and WO2019/143636; U.S. Pat. Nos. 5,731,168; 5,807,706; 5,821,333; and U.S. Appl. Publ. Nos. 2003/020734, 2002/0155537, 2014/242079, 2015/133640, 2016/297885 and 2017/037130. Bispecific tetravalent antibodies, and methods of making them are described, e.g., in WO 02/096948 and WO 00/44788, the disclosures of both of which are herein incorporated by reference in its entirety. In addition, other publications relating to making bispecific antibodies include WO 91/00360; WO 92/08802; WO 92/05793, and WO 93/17715; Tutt et al., J. Immunol. 147:60-69 (1991); U.S. Pat. Nos. 4,474,893; 4,714,681; 4,925,648; 5,573,920; 5,601,819, 9,212,230 and 9,701,759; and Kostelny et al., J. Immunol. 148:1547-1553 (1992).

In various embodiments, the anti-IL-10Rα VHH, the anti-IL-10Rβ VHH, the anti-IL-10Rαβ VHH1-VHH2, or the anti-IL-10Rαβ VHH1-VHH2-Fc molecule expressed from the expression cassettes, described herein, may be produced in prokaryotic cells (e.g., bacterial cells, e.g., E. coli cells), eukaryotic cells, e.g., yeast cells, plant cells, mammalian cells, e.g., CHO-based or CHO-originated cells. The anti-IL-10Rα VHH, the anti-IL-10Rβ VHH, the anti-IL-10Rαβ VHH1-VHH2, or the anti-IL-10Rαβ VHH1-VHH2-Fc molecules can also be produced in eukaryotic cells such as transformed cell lines (e.g., 293E, 293T, Expi293™, COS, NIH3T3). In addition, the anti-IL-10Rα VHH, the anti-IL-10Rβ VHH, the anti-IL-10Rαβ VHH1-VHH2, or the anti-IL-10Rαβ VHH1-VHH2-Fc molecules described herein can be expressed in a yeast cell such as Pichia (see, e.g., Powers et al., J Immunol Methods. 251:123-35 (2001)), Hanseula, or Saccharomyces. In one embodiment, the anti-IL-10Rα VHH, the anti-IL-10Rβ VHH, the anti-IL-10Rαβ VHH1-VHH2, or the anti-IL-10Rαβ VHH1-VHH2-Fc molecules described herein are produced in a CHO-based or CHO-originated cell line (e.g., CHO-S, CHO DG44, CHO DXB-11, ExpiCHO™, CHOZN® ZFN-modified GS−/− CHO cell line, CHOSOURCE™ GS knockout cell line, CHOK1SV, CHOK1SV GS-KO® cell line, CHO-K1, or other CHO-K1-derived cell lines) or a HEK293 (e.g., Expi293™) cell line. To produce the anti-IL-10Rα VHH, the anti-IL-10Rβ VHH, the anti-IL-10Rαβ VHH1-VHH2, or the anti-IL-10Rαβ VHH1-VHH2-Fc molecules, one or more polynucleotides encoding the anti-IL-10Rα VHH, the anti-IL-10Rβ VHH, the anti-IL-10Rαβ VHH1-VHH2, or the anti-IL-10Rαβ VHH1-VHH2-Fc molecules are constructed, introduced into an expression vector, and then expressed in one or more suitable host cells. Standard molecular biology techniques can be used to prepare the recombinant expression vector, transfect the host cells, select for transfectants, culture the transfected cells, and recover the anti-IL-10Rα VHH, the anti-IL-10Rβ VHH, the anti-IL-10Rαβ VHH1-VHH2, or the anti-IL-10Rαβ VHH1-VHH2-Fc molecules. As appropriate or desired, the cell or population of cells are cultured in a culture volume of at least 2 L, e.g., at least 5 L, 10 L, 50 L, 100 L, 150 L, 200 L, 250 L, or more.

If the anti-IL-10Rα VHH, the anti-IL-10Rβ VHH, the anti-IL-10Rαβ VHH1-VHH2, or the anti-IL-10Rαβ VHH1-VHH2-Fc molecule is to be expressed in mammalian host cells, e.g., such as CHO-based or CHO-originated cells, COS, and NIH3T3 cells, the expression vector includes a promoter useful for expression in these cells. In various embodiments, the promoter for expression of the anti-IL-10Rα VHH, the anti-IL-10Rβ VHH, the anti-IL-10Rα VHH1-VHH2, or the anti-IL-10Rαβ VHH1-VHH2-Fc molecule in mammalian cells is a constitutive promoter or an inducible promoter. Illustrative promoters for expression of the anti-IL-10Rα VHH, the anti-IL-10Rβ VHH, the anti-IL-10Rαβ VHH1-VHH2, or the anti-IL-10Rα VHH1-VHH2-Fc molecule in mammalian cells include without limitation those described above, and e.g., an SV40 promoter (Mulligan et al., Nature, 277:108 (1979)), a MMLV-LTR promoter, an EF1α promoter (Mizushima et al., Nucleic Acids Res., 18:5322 (1990)), or a CMV promoter. In addition to the nucleic acid sequence encoding the anti-IL-10Rα VHH, the anti-IL-10Rβ VHH, the anti-IL-10Rαβ VHH1-VHH2, or the anti-IL-10Rαβ VHH1-VHH2-Fc molecule thereof, the recombinant expression vectors may carry additional sequences, such as sequences that regulate replication of the vector in host cells (e.g., origins of replication) and selection marker genes. The selection marker gene facilitates selection of host cells into which the vector has been introduced (see e.g., U.S. Pat. Nos. 4,399,216, 4,634,665 and 5,179,017). In some embodiments, the selection marker gene confers resistance to drugs, such as G418, hygromycin, zeocin, blastcidin, puromycin, or methotrexate, on a host cell into which the vector has been introduced. Examples of vectors with selection markers include pMAM, pDR2, pBK-RSV, pBK-CMV, pOPRSV, pIRESbleo3, pIRESneo3, pIREShyg3, pIRESpuro3, and pOP13. In some embodiments, the selection marker is a glutamine synthetase (GS).

In one embodiment, the anti-IL-10Rα VHH, the anti-IL-10Rβ VHH, the anti-IL-10Rαβ VHH1-VHH2, or the anti-IL-10Rαβ VHH1-VHH2-Fc molecule are produced in mammalian cells. Exemplary mammalian host cells for expressing the anti-IL-10Rα VHH, the anti-IL-10Rβ VHH, the anti-IL-10Rαβ VHH1-VHH2, or the anti-IL-10Rαβ VHH1-VHH2-Fc molecules include Chinese Hamster Ovary (CHO cells) (including dhfr-CHO cells, e.g., described in Urlaub and Chasin (1980) Proc. Natl. Acad. Sci. USA 77: 4216-4220, used with a DHFR selection marker, e.g., as described in Kaufman and Sharp (1982) Mol. Biol. 159:601 621 and glutamine synthetase (GS)-cells used with a GS selection marker, e.g., described in Lin, et al., MAbs. (2019) 11(5):965-976; and Noh, et al., Sci Rep. (2018) 8(1):5361), human embryonic kidney 293 cells (e.g., 293, 293E, 293T, Expi293™), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell. CHO and NS0 cell lines for recombinant antibody production are reviewed by Dhara, et al., BioDrugs. (2018) 32(6):571-584.

In an exemplary system for expression of the anti-IL-10Rα VHH, the anti-IL-10Rβ VHH, the anti-IL-10Rαβ VHH1-VHH2, or the anti-IL-10Rαβ VHH1-VHH2-Fc molecules, recombinant expression vectors are introduced into dhfr-CHO cells by calcium phosphate-mediated transfection. In a specific embodiment, the dhfr-CHO cells are cells of the DG44 cell line, such as DG44i (see, e.g., Derouaz et al., Biochem Biophys Res Commun., (2006) 340(4):1069-77). Within the recombinant expression vectors, the first and second polypeptide chains are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vectors also carry a DHFR gene, which allows for selection of CHO cells that have been transfected with the vector using methotrexate selection/amplification. The selected transformant host cells are cultured to allow for expression of the anti-IL-10Rα VHH, the anti-IL-10Rβ VHH, the anti-IL-10Rαβ VHH1-VHH2, or the anti-IL-10Rαβ VHH1-VHH2-Fc molecule, which can be recovered from the culture medium.

The anti-IL-10Rα VHH, the anti-IL-10Rβ VHH, the anti-IL-10Rαβ VHH1-VHH2, or the anti-IL-10Rαβ VHH1-VHH2-Fc molecules can be isolated from inside or outside (such as medium) of the host cell and purified as substantially pure and homogenous, non-aggregated molecules. In various embodiments, the anti-IL-10Rα VHH, the anti-IL-10Rβ VHH, the anti-IL-10Rαβ VHH1-VHH2, or the anti-IL-10Rαβ VHH1-VHH2-Fc molecules described herein, and/or the polynucleotides encoding such polypeptides, are provided in provided in isolated form. This means that such the polypeptide or polynucleotide is at least 50% w/w pure of interfering proteins, cellular and other contaminants arising from its production or purification but does not exclude the possibility that the agent is combined with an excess of pharmaceutical acceptable carrier(s) or other vehicle intended to facilitate its use. The term “isolated,” when applied to a polypeptide or polynucleotide, as described herein, denotes that the polypeptide or polynucleotide is essentially free of cellular components with which it is associated in the natural state. It can be, for example, in a homogeneous state and may be in either a dry or aqueous solution. Purity and homogeneity can be determined using known methods, e.g., analytical chemistry techniques such as polyacrylamide gel electrophoresis, column chromatography, thin layer chromatography, or high-performance liquid chromatography (HPLC) analysis. A protein that is the predominant species present in a preparation is substantially purified. An “isolated” or “purified” polypeptide or polynucleotide is substantially free of other cellular material, or culture medium when produced by recombinant techniques, or chemical precursors or other chemicals when chemically synthesized. In various embodiments, purified polypeptides and/or polynucleotides are at least 60%, 70%, 75%, 80%, 85%, 90%, 91%, 92%, 93%, 94%, 95%, 96%, 97%, 98%, 99%, or 100% (w/w), separated from, purified of, or free of interfering proteins and contaminants from production or purification. As appropriate or desired, the anti-IL-10Rα VHH, the anti-IL-10Rβ VHH, the anti-IL-10Rαβ VHH1-VHH2, or the anti-IL-10Rαβ VHH1-VHH2-Fc molecule, described herein, is the predominant macromolecular species remaining after its purification.

Methods for isolation and purification commonly used for antibody purification may be used for the isolation and purification of the anti-IL-10Rα VHH, the anti-IL-10Rβ VHH, the anti-IL-10Rαβ VHH1-VHH2, or the anti-IL-10Rαβ VHH1-VHH2-Fc molecules, and are not limited to any particular method. The anti-IL-10Rα VHH, the anti-IL-10Rβ VHH, the anti-IL-10Rαβ VHH1-VHH2, or the anti-IL-10Rαβ VHH1-VHH2-Fc molecules may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include Protein A column and protein G column. Examples of columns using Protein A column include Hyper D, POROS, and Sepharose FF (GE Healthcare Biosciences). The present disclosure also includes anti-IL-10Rα VHH, anti-IL-10Rβ VHH, anti-IL-10Rαβ VHH1-VHH2, or anti-IL-10Rαβ VHH1-VHH2-Fc molecules that are highly purified using these purification methods. In various embodiments, the isolating or purifying step comprises Protein A chromatography, and at least 90%, 91%, 92%, 93%, 94%, 95%, 96%, 97%, 98%, 99%, or more, of the anti-IL-10Rα VHH, the anti-IL-10Rβ VHH, the anti-IL-10Rαβ VHH1-VHH2, or the anti-IL-10Rαβ VHH1-VHH2-Fc molecules are isolated or purified. In various embodiments, the isolating or purifying step comprises Protein A chromatography, followed by size exclusion or ion exchange chromatography, and at least 95%, 96%, 97%, 98%, 99%, or more, of the anti-IL-10Rα VHH, the anti-IL-10Rβ VHH, the anti-IL-10Rαβ VHH1-VHH2, or the anti-IL-10Rαβ VHH1-VHH2-Fc molecules are isolated or purified. In various embodiments, at least 95%, 96%, 97%, 98%, 99%, or more, of the anti-IL-10Rα VHH, the anti-IL-10Rβ VHH, the anti-IL-10Rαβ VHH1-VHH2, or the anti-IL-10Rαβ VHH1-VHH2-Fc molecules isolate or purify as non-aggregated soluble heterodimer as determined using size exclusion chromatography (SEC). In some embodiments, the isolated or purified anti-IL-10Rα VHH, anti-IL-10Rβ VHH, anti-IL-10Rαβ VHH1-VHH2, or anti-IL-10Rαβ VHH1-VHH2-Fc molecules have increased homogeneity as assessed by analytical ion exchange chromatography, wherein the integrated area of a main peak representing an unmodified target species is at least 95%, 96%, 97%, 98%, or more, of the sum of all integrated protein peak areas. In some embodiments, the isolated or purified antigen binding molecules have fewer than 35%, 30%, 25%, 20%, 15%, 10%, 9%, 8%, 7%, or fewer, acidic contaminants.

### 9. Pharmaceutical Compositions

Further provided are pharmaceutical compositions comprising the anti-IL-10Rα VHH, the anti-IL-10Rβ VHH, the anti-IL-10Rαβ VHH1-VHH2, or the anti-IL-10Rαβ VHH1-VHH2-Fc, as described herein, or one or more polynucleotide encoding the anti-IL-10Rα VHH, the anti-IL-10Rβ VHH, the anti-IL-10Rαβ VHH1-VHH2, or the anti-IL-10Rαβ VHH1-VHH2-Fc, as described herein, and a pharmaceutically acceptable diluent, carrier or excipient. In certain embodiments, the pharmaceutical composition comprises a therapeutically effective amount of one or more unitary doses of the anti-IL-10Rα VHH, the anti-IL-10Rβ VHH, the anti-IL-10Rαβ VHH1-VHH2, or the anti-IL-10Rαβ VHH1-VHH2-Fc, or one or more polynucleotides encoding the anti-IL-10Rα VHH, the anti-IL-10Rβ VHH, the anti-IL-10Rαβ VHH1-VHH2, or the anti-IL-10Rαβ VHH1-VHH2-Fc.

Various pharmaceutically acceptable diluents, carriers, and excipients, and techniques for the preparation and use of pharmaceutical compositions will be known to those of skill in the art in light of the present disclosure. Illustrative pharmaceutical compositions and pharmaceutically acceptable diluents, carriers, and excipients are also described, e.g., in Remington: The Science and Practice of Pharmacy 20th Ed. (Lippincott, Williams & Wilkins 2003); Loyd V. Allen Jr (Editor), “Remington: The Science and Practice of Pharmacy,” 22^{nd} Edition, 2012, Pharmaceutical Press; Brunton, Knollman and Hilal-Dandan, “Goodman and Gilman's The Pharmacological Basis of Therapeutics,” 13th Edition, 2017, McGraw-Hill Education/Medical; McNally and Hastedt (Editors), “Protein Formulation and Delivery, 2nd Edition, 2007, CRC Press; Banga, “Therapeutic Peptides and Proteins: Formulation, Processing, and Delivery Systems,” 3rd Edition, 2015, CRC Press; Lars Hovgaard, Frokjaer and van de Weert (Editors), “Pharmaceutical Formulation Development of Peptides and Proteins,” 2nd Edition, 2012, CRC Press; Carpenter and Manning (Editors), “Rational Design of Stable Protein Formulations: Theory and Practice,” 2002, Springer (Pharmaceutical Biotechnology (Book 13)); Meyer (Editor), “Therapeutic Protein Drug Products: Practical Approaches to Formulation in the Laboratory, Manufacturing, and the Clinic, 2012, Woodhead Publishing; and Shire, “Monoclonal Antibodies: Meeting the Challenges in Manufacturing, Formulation, Delivery and Stability of Final Drug Product, 2015, Woodhead Publishing.

In some embodiments, each carrier, diluent or excipient is “acceptable” in the sense of being compatible with the other ingredients of the pharmaceutical composition and not injurious to the subject. Often, the pharmaceutically acceptable carrier is an aqueous pH-buffered solution. Some examples of materials which can serve as pharmaceutically-acceptable carriers, diluents or excipients include: water; buffers, e.g., phosphate-buffered saline; sugars, such as lactose, trehalose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; 2-Amino-2-(hydroxymethyl)propane-1,3-diol (i.e., tris(hydroxymethyl)aminomethane; Tris) buffers, amino acids (e.g., charged amino acids, including without limitation, aspartate, asparagine, glutamate, glutamine, histidine, lysine, arginine); and other non-toxic compatible substances employed in pharmaceutical formulations. Wetting agents, emulsifiers and lubricants, such as sodium lauryl sulfate, a polysorbate (e.g., polysorbate 20, polysorbate 40, polysorbate 60, polysorbate 80) and magnesium stearate, as well as coloring agents, release agents, coating agents, sweetening, flavoring and perfuming agents, preservatives and antioxidants can also be present in the compositions. In certain embodiments, pharmaceutical compositions are sterile. In certain embodiments, the pharmaceutical composition has a pH in the range of 4.5 to 8.5, 5.5 to 7.4, 4.5 to 6.5, 6.4 to 7.0, 6.5 to 8.5, 7.2 to 7.8, or a pH of 5.0, 5.5, 5.6, 5.7, 5.8, 5.9, 6.0, 6.5, 7.0, 7.5, 8.0 or 8.5. In one embodiment, the pharmaceutical composition comprises a physiologically acceptable buffer, pH 5.5 to 8.5, e.g., pH 5.5 to 6.5. In one embodiment, the pharmaceutical composition has an osmolarity in the range of 240-260 or 250-330 mOsmol/L. In certain embodiments, the pharmaceutical composition is isotonic or near isotonic.

The formulation of and delivery methods of pharmaceutical compositions will generally be adapted according to the site and the disease to be treated. Exemplary formulations include, but are not limited to, those suitable for enteral administration (per os) or parenteral administration, e.g., subcutaneous, intravenous, intra-arterial, or intramuscular, including formulations encapsulated in micelles, liposomes or drug-release capsules (active agents incorporated within a biocompatible coating designed for slow-release); ingestible formulations; formulations for topical use, such as creams, ointments and gels; and other formulations such as inhalants, aerosols and sprays.

In some embodiments, the pharmaceutical compositions are formulated for parenteral, e.g., intravenous, subcutaneous, or intramuscular). In particular embodiments, for parenteral administration, the antibodies or antigen-binding fragments thereof are formulated in a unit dosage injectable form (solution, suspension, emulsion) in association with a pharmaceutically acceptable, parenteral vehicle. Examples of such vehicles include water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. In certain embodiments, the anti-IL-10Rα VHH, the anti-IL-10Rβ VHH, the anti-IL-10Rαβ VHH1-VHH2, or the anti-IL-10RaP VHH1-VHH2-Fc are formulated in such vehicles at concentrations of from 5 mg/ml to 150 mg/ml.

### 10. Methods of Treating IL-10R-Mediated Inflammatory Conditions

Further provided are methods of preventing or treating an inflammatory disease in a subject in need thereof, comprising administering to the subject an effective amount of an anti-IL-10Rαβ VHH1-VHH2, or an anti-IL-10Rαβ VHH1-VHH2-Fc, as described herein. Also provided are methods of reducing the severity of or mitigating on or more symptoms of an inflammatory disease in a subject in need thereof, comprising administering to the subject an effective amount of an anti-IL-10Rαβ VHH1-VHH2, or an anti-IL-10Rαβ VHH1-VHH2-Fc, as described herein.

The term “treatment” or “treating,” to the extent it relates to a disease or condition, includes one or more of preventing the disease or condition from occurring, inhibiting the disease or condition, eliminating the disease or condition, and relieving one or more symptoms of the disease or condition. The terms “treating” and “treatment” as used herein are intended to mean the administration of a compound or composition according to the embodiments disclosed herein to at least one of alleviate or eliminate symptoms of the inflammatory disease in a patient. As used herein, the term “effective amount” in the context of the administration of a therapy to a subject refers to the amount of a therapy that achieves a desired prophylactic or therapeutic effect. Administration of a preventative or prophylactic agent can occur prior to the manifestation of symptoms characteristic of the inflammatory disease or disorder, such that a disease or disorder is prevented or, alternatively, delayed in its progression.

For in vivo treatment of mammalian subject, e.g., humans, the subject may be administered or provided a pharmaceutical composition comprising an anti-IL-10Rαβ VHH1-VHH2, or an anti-IL-10Rαβ VHH1-VHH2-Fc, as described herein, described herein, and optionally an additional anti-inflammatory agent. When used for in vivo therapy, an antibody or antibodies described herein are typically administered or provided to the patient in therapeutically effective amounts (i.e., amounts that eliminate or reduce inflammatory lesions or inflammatory mediators). In certain therapeutic applications, the anti-IL-10Rαβ VHH1-VHH2, or the anti-IL-10Rαβ VHH1-VHH2-Fc is administered to a patient already suffering from an inflammatory disease or condition, in an amount sufficient to mitigate or ameliorate or at least partially arrest at least one of the symptoms of the inflammatory disease or condition. Amounts effective for this use depend on the severity and course of the disease or condition, previous therapy, the patient's health status, weight, and response to the anti-IL-10Rαβ VHH1-VHH2, or the anti-IL-10Rαβ VHH1-VHH2-Fc, and the judgment of the treating physician. Therapeutically effective amounts are optionally determined by methods including, but not limited to, a dose escalation and/or dose ranging clinical trial. The amount of a administered anti-IL-10Rαβ VHH1-VHH2, or anti-IL-10Rαβ VHH1-VHH2-Fc that corresponds to such an amount varies depending upon factors such as the particular anti-IL-10Rαβ VHH1-VHH2, or an anti-IL-10Rαβ VHH1-VHH2-Fc, inflammatory disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated. In some embodiments, an anti-IL-10Rαβ VHH1-VHH2, or an anti-IL-10Rαβ VHH1-VHH2-Fc, as described herein, is administered at a dose of from 0.5 μg/kg to 50 mg/kg body weight per administration. In some embodiments, dosing of an anti-IL-10Rαβ VHH1-VHH2, or an anti-IL-10Rαβ VHH1-VHH2-Fc, as described herein, described herein, can be reduced or decreased when combining with a second therapeutic agent, e.g., improving the effectiveness or efficacy of an agent that is standard of care.

In some embodiments, an anti-IL-10Rαβ VHH1-VHH2, or an anti-IL-10Rαβ VHH1-VHH2-Fc, as described herein, has a serum half-life in a human of at least 3 days, e.g., at least 4 days, at least 5 days, at least 6 days, at least 7 days, at least 8 days, at least 9 days, at least 10 days, at least 11 days, at least 12 days, or longer.

In various embodiments, an anti-IL-10Rαβ VHH1-VHH2, or an anti-IL-10Rαβ VHH1-VHH2-Fc, as described herein, is administered one or multiple times. In embodiments employing multiple administration regimens, as appropriate the anti-IL-10Rαβ VHH1-VHH2 or the anti-IL-10Rαβ VHH1-VHH2-Fc, can be administered in once daily, once weekly (i.e., QW), once bi-weekly (i.e. once every other week, or once every two weeks or Q2W), once every three weeks (i.e., Q3W), once monthly (i.e., QM or Q4W) or once bi-monthly (i.e. once every other month, or once every two months or Q2M or Q8W) dosing or administration intervals. In some embodiments, the anti-IL-10Rαβ VHH1-VHH2 or the anti-IL-10Rαβ VHH1-VHH2-Fc, are administered intravenously, subcutaneously or intramuscularly. As appropriate, doses administered in multiple administration regimens can be the same or different between the first and subsequent doses of the anti-IL-10Rαβ VHH1-VHH2 or the anti-IL-10Rαβ VHH1-VHH2-Fc. As appropriate, doses administered in multiple administration regimens can be the same or different between the anti-IL-10Rαβ VHH1-VHH2 or the anti-IL-10Rαβ VHH1-VHH2-Fc, and a second therapeutic agent.

As appropriate, the anti-IL-10Rαβ VHH1-VHH2 or the anti-IL-10Rαβ VHH1-VHH2-Fc, are administered or provided to a mammalian subject, e.g., a human, in accordance with known methods, such as, but not limited to, intravenous administration, e.g., as a bolus or by continuous infusion over a period of time, by intramuscular, subcutaneous, intraperitoneal, intracerebrospinal, intraarticular, intrasynovial, intrathecal, oral, topical, or inhalation routes. As appropriate, the anti-IL-10Rαβ VHH1-VHH2 or the anti-IL-10Rαβ VHH1-VHH2-Fc, may be administered parenterally, when possible, at the target cell site, or intravenously. In one embodiment, administration of the anti-IL-10Rαβ VHH1-VHH2 or the anti-IL-10Rα VHH1-VHH2-Fc to the subject is via an intravenous route. In another embodiment, administration of the anti-IL-10Rαβ VHH1-VHH2 or the anti-IL-10Rαβ VHH1-VHH2-Fc to the subject is via a subcutaneous route. In another embodiment, administration of the anti-IL-10Rαβ VHH1-VHH2 or the anti-IL-10Rαβ VHH1-VHH2-Fc to the subject is via an intramuscular route. In various embodiments, polynucleotides encoding the anti-IL-10Rαβ VHH1-VHH2 or the anti-IL-10Rα VHH1-VHH2-Fc can be electroporated, e.g., for transdermal delivery. In some embodiments, pharmaceutical compositions of the disclosure are administered to a subject systemically (e.g., enterally, orally, per os), parenterally, or locally.

In some embodiments, the anti-IL-10Rαβ VHH1-VHH2 or the anti-IL-10Rαβ VHH1-VHH2-Fc is administered via a device. In some embodiments, the device is configured for sustained delivery. In some embodiments, the device is configured for self-administration. In some embodiments, the device is a prefilled syringe. In some embodiments, the device is an ingestible device configured for local delivery to the intestine. Ingestible devices for delivering an IL-10R agonist to the gastrointestinal tract are described, e.g., in U.S. Pat. No. 11,596,670, and can be used for delivery of the anti-IL-10Rαβ VHH1-VHH2 or the anti-IL-10Rαβ VHH1-VHH2-Fc, described herein.

In some embodiments, the inflammatory disease is mediated by an IL-10 deficiency or deficient signaling through the IL-10R. In some embodiments, the inflammatory disease is an autoimmune disease. In some embodiments, the inflammatory disease is an inflammatory bowel disease, e.g., ulcerative colitis, Crohn's disease, mucosal proctocolitis and celiac disease. In some embodiments, the inflammatory disease is an inflammatory dermatitis disease. In some embodiments, the inflammatory dermatitis disease is selected from atopic dermatitis (AD), hidradenitis suppurativa (HS), and psoriasis. In some embodiments, the inflammatory disease is selected from ulcerative colitis, Crohn's disease, mucosal proctocolitis and celiac disease, rheumatoid arthritis (RA), juvenile idiopathic arthritis, atopic dermatitis (AD), hidradenitis suppurativa (HS), psoriasis, systemic lupus erythematosus (SLE), Behget's disease, type 1 diabetes, primary sclerosing cholangitis, ankylosing spondylitis, and multiple sclerosis. In various embodiments, the inflammatory disease is acute or chronic.

### 11. Combination Therapies

In some embodiments, the anti-IL-10Rαβ VHH1-VHH2, or the anti-IL-10Rαβ VHH1-VHH2-Fc, described herein is combined or co-administered with one or more (e.g., one, two, three, or four) additional therapeutic agents. In some embodiments, the one or more additional agents are selected from infliximab, adalimumab, golimumab, vedolizumab, tofacitinib, ustekinumab, natalizumab, mesalamine, diazo-bonded 5-ASA, sulfasalazine, balsalazide, olsalazine, a corticosteroid (e.g., budesonide, hydrocortisone, methylprednisolone, prednisone); an immunosuppressant (e.g., azathioprine, 6-mercaptopurine, cyclosporine, methotrexate), and combinations thereof. In some embodiments, the one or more additional agents are selected from a PPARδ inhibitor, an IRAK4 inhibitor, a TPL2 inhibitor, an α4β7 inhibitor, a BTLA agonist (e.g. as described in WO 2020/128446 and WO 2021/250419), a PD-1 agonist (e.g., WO 2023/089377), a CD200R agonist (e.g., as described in WO 2023/196866) or a FXR agonist. In some embodiments, the one or more additional agents are selected from seladelpar, edecesertib, tilpisertib fosmecarbil, GS-1427, GS-0272, GS-0151, GS-6791 and cilofexor.

In some embodiments, the additional therapeutic agent includes an agent useful for modulating, treating, or preventing inflammation, such as a 4-1BB ligand, 4-1BB ligand modulator, 5-HT 2a receptor antagonist, 5-HT 2b receptor antagonist, 5-HT 3 receptor antagonist, 5-HT 4 receptor agonist, 5-HT receptor agonist, 5-Lipoxygenase inhibitor, Acetaldehyde dehydrogenase inhibitor, Actin antagonist, ADAMTS inhibitor, ADAMTS4 gene inhibitor, Adenosine A1 receptor antagonist, Adenosine A2a receptor agonist, Adenosine A3 receptor agonist, Adenosine A3 receptor modulator, Adenosine deaminase stimulator, Adenosylhomocysteinase inhibitor, Adiponectin receptor modulator, ADP ribosyl cyclase-1 inhibitor, ADP ribosyl cyclase-1 modulator, ADP ribosylation factor 6 inhibitor, Adrenocorticotrophic hormone ligand, Aggrecan modulator, Aggrecanase-1 inhibitor, Aggrecanase-2 inhibitor, AIMP multisynthetase complex protein 1 stimulator, Albumin antagonist, Albumin modulator, Alk-2 protein kinase inhibitor, Alk-5 protein kinase inhibitor, Alpha crystallin chain B stimulator, Aminopeptidase N modulator, Amylin receptor agonist, Angiopoietin-related protein-3 stimulator, Angiotensin II AT-2 receptor agonist, Annexin A1 inhibitor, Annexin A2 modulator, AP1 transcription factor inhibitor, AP1 transcription factor modulator, Apelin receptor agonist, Apolipoprotein B modulator, APRIL receptor antagonist, APRIL receptor modulator, Aryl hydrocarbon receptor agonist, B and T lymphocyte attenuator stimulator, Basigin inhibitor, Bcl-2 protein inhibitor, Beta 2 adrenoceptor agonist, Beta amyloid antagonist, Beta-catenin modulator, Bifunctional aminoacyl tRNA synthetase inhibitor, B-lymphocyte antigen CD19 inhibitor, B-lymphocyte antigen CD19 modulator, B-lymphocyte antigen CD20 inhibitor, B-lymphocyte antigen CD20 modulator, B-lymphocyte cell adhesion molecule inhibitor, B-lymphocyte stimulator ligand inhibitor, B-lymphocyte stimulator ligand modulator, Bone morphogenetic protein-7 ligand, Bone morphogenetic protein-7 ligand modulator, Brain derived neurotrophic factor ligand inhibitor, Branched amino acid aminotransferase 1 inhibitor, Bromodomain containing protein inhibitor, Btk tyrosine kinase inhibitor, CACNA2D1 calcium channel subunit inhibitor, Cak tyrosine kinase receptor inhibitor, Calcineurin inhibitor, Calcitonin receptor agonist, Calcium channel inhibitor, Calcium channel inhibitor L-type, Calreticulin inhibitor, Cannabinoid CB1 receptor inverse agonist, Cannabinoid CB2 receptor agonist, Cannabinoid receptor agonist, Cannabinoid receptor modulator, Carbonic anhydrase inhibitor, Carbonic anhydrase-VIII stimulator, Caspase inhibitor, Cathepsin K inhibitor, CCR2 chemokine antagonist, CCR3 chemokine modulator, CCR3 gene modulator, CCR5 chemokine antagonist, CCR6 chemokine antagonist, CD11b agonist, CD11b antagonist, CD223 agonist, CD226 antigen inhibitor, CD29 modulator, CD3 modulator, CD30 modulator, CD4 modulator, CD40 ligand inhibitor, CD40 ligand receptor antagonist, CD45RC modulator, CD47 antagonist, CD49e modulator, CD70 antigen modulator, CD79b modulator, CD80 antagonist, CD80 modulator, CD86 antagonist, CD86 modulator, CDw123 modulator, Cell division control 42 homolog inhibitor, Cell surface glycoprotein MUC18 inhibitor, Chaperonin modulator, Chemokine receptor-like 2 modulator, Cholesterol esterase inhibitor, Choline kinase inhibitor, CLIP associating protein 2 inhibitor, CLK2 protein kinase inhibitor, Collagen I agonist, Collagen I antagonist, Collagen II antagonist, Collagen modulator, Collagen VII antagonist, Complement C1q subcomponent inhibitor, Complement C3 inhibitor, Complement C5 factor inhibitor, Complement C5a receptor antagonist, Complement cascade inhibitor, Complement cascade modulator, Complement Factor B inhibitor, Complement factor C2 inhibitor, Complement factor D inhibitor, Complement Factor inhibitor, C-type lectin domain protein 2D inhibitor, C-type lectin domain protein 4C inhibitor, C-type lectin domain protein 4C modulator, CXCR3 chemokine antagonist, CXCR4 chemokine modulator, CXCR5 chemokine antagonist, CXCR5 chemokine modulator, cyclic GMP AMP synthase inhibitor, Cyclin-dependent kinase inhibitor 1 inhibitor, Cyclin-dependent kinase-2 inhibitor, Cyclin-dependent kinase-4 inhibitor, Cyclin-dependent kinase-5 inhibitor, Cyclin-dependent kinase-6 inhibitor, Cyclin-dependent kinase-7 inhibitor, Cyclin-dependent kinase-9 inhibitor, Cyclooxygenase 1 inhibitor, Cyclooxygenase 2 inhibitor, Cyclooxygenase inhibitor, Cytotoxic T-lymphocyte protein-4 modulator, Cytotoxic T-lymphocyte protein-4 stimulator, Deoxycytidine kinase inhibitor, Deoxyribonuclease gamma stimulator, Deoxyribonuclease I stimulator, Deoxyribonuclease modulator, DHFR inhibitor, Dihydroorotate dehydrogenase inhibitor, Dipeptidyl peptidase I inhibitor, Dipeptidyl peptidase IV inhibitor, DNA binding protein Ikaros inhibitor, DYRK-1 alpha protein kinase inhibitor, Early activation antigen CD69 inhibitor, EBV induce G protein coupled receptor 2 antagonist, Ectonucleotide pyrophosphatase-PDE-2 inhibitor, Elastin stimulator, Endoplasmin inhibitor, Endostatin modulator, Endothelin ET-A receptor antagonist, Endothelin ET-B receptor antagonist, Enolase 1 inhibitor, Eotaxin 2 ligand inhibitor, EP4 prostanoid receptor antagonist, Ephb2 tyrosine kinase receptor inhibitor, Ephrin B2 ligand inhibitor, Epidermal growth factor ligand inhibitor, Epidermal growth factor ligand modulator, Erbb3 tyrosine kinase receptor modulator, Erythropoietin receptor agonist, Exportin 1 inhibitor, Extracellular matrix protein modulator, Extracellular matrix protein stimulator, Factor XIIa antagonist, FGF receptor antagonist, FGF18 gene stimulator, FGF-18 ligand, FGF-2 ligand inhibitor, FK506 binding protein inhibitor, FMLP related receptor I agonist, FMLP related receptor II agonist, Folate receptor antagonist, Folate receptor modulator, G protein coupled receptor 15 antagonist, GABA receptor agonist, Gap junction alpha-1 protein inhibitor, Glucagon-like peptide 1 receptor agonist, Glucocorticoid receptor agonist, Glucocorticoid receptor modulator, GM-CSF ligand inhibitor, GM-CSF receptor modulator, GMP synthetase inhibitor, GroEL protein 2 inhibitor, Growth differentiation factor-5 modulator, Growth hormone receptor agonist, Growth hormone receptor antagonist, Guanylate cyclase receptor agonist, Guanylate cyclase stimulator, H+ K+ ATPase inhibitor, Heat shock protein gene stimulator, Histamine H4 receptor antagonist, Histone deacetylase inhibitor, Histone deacetylase-6 inhibitor, Histone H2A modulator, Histone H4 modulator, HLA antigen modulator, HLA class II antigen DR-1 beta inhibitor, HLA class II antigen inhibitor, HLA class II antigen modulator, Hyaluronic acid receptor agonist, Hyaluronic acid receptor antagonist, Hyaluronic acid receptor modulator, Hyaluronidase stimulator, Hypoxia inducible factor stimulator, IFNB gene stimulator, IgG receptor FcRn large subunit p51 antagonist, IgG receptor FcRn large subunit p51 modulator, I-kappa B kinase beta inhibitor, I-kappa B kinase epsilon inhibitor, Ikaros family zinc finger protein inhibitor, IL-1 receptor accessory protein inhibitor, IL-1 receptor antagonist, IL-10 receptor agonist, IL-10 receptor antagonist, IL-12 receptor antagonist, IL-13 receptor modulator, IL-17 antagonist, IL-18 antagonist, IL-18 receptor accessory protein antagonist, IL1RA gene modulator, IL-2 receptor agonist, IL-2 receptor alpha subunit inhibitor, IL-2 receptor antagonist, IL-2 receptor modulator, IL-23 antagonist, IL-3 receptor antagonist, IL-3 receptor modulator, IL-4 receptor modulator, IL-6 receptor agonist, IL-6 receptor antagonist, IL-6 receptor modulator, IL-7 receptor modulator, Immunoglobulin agonist, Immunoglobulin antagonist, Immunoglobulin G agonist, Immunoglobulin G binding protein A modulator, Immunoglobulin G1 agonist, Immunoglobulin G1 modulator, Immunoglobulin gamma Fc receptor antagonist, Immunoglobulin gamma Fc receptor II modulator, Immunoglobulin gamma Fc receptor IIB agonist, Immunoglobulin gamma Fc receptor IIB modulator, Immunoglobulin modulator, Indoleamine-pyrrole-2,3-dioxygenase stimulator, Inducible nitric oxide synthase inhibitor, Inducible T-cell co-stimulator inhibitor, Inosine monophosphate dehydrogenase inhibitor, Insulin-like growth factor 1 receptor antagonist, Insulin-like growth factor 1 receptor modulator, Integrin alpha-4/beta-1 antagonist, Integrin alpha-V/beta-3 antagonist, Interferon alpha 1 ligand inhibitor, Interferon alpha 2 ligand, Interferon alpha ligand, Interferon alpha ligand inhibitor, Interferon alpha ligand modulator, Interferon beta ligand inhibitor, Interferon gamma ligand, Interferon gamma ligand inhibitor, Interferon type I receptor antagonist, Interleukin 1 delta ligand inhibitor, Interleukin 1 like receptor 2 inhibitor, Interleukin 17 ligand inhibitor, Interleukin 17A ligand inhibitor, Interleukin 17A ligand modulator, Interleukin 17F ligand inhibitor, Interleukin 17F ligand modulator, Interleukin 18 ligand inhibitor, Interleukin 23A inhibitor, Interleukin 33 ligand inhibitor, Interleukin ligand, Interleukin receptor 17A antagonist, Interleukin-1 alpha ligand inhibitor, Interleukin-1 beta ligand inhibitor, Interleukin-1 beta ligand modulator, Interleukin-1 receptor antagonist inhibitor, Interleukin-10 ligand, Interleukin-2 ligand, Interleukin-31 receptor modulator, Interleukin-6 ligand inhibitor, IRAK-1 protein kinase inhibitor, IRAK-4 protein kinase inhibitor, Itk tyrosine kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, KCNA voltage-gated potassium channel-3 inhibitor, KDM6B gene inhibitor, Kelch like ECH associated protein 1 modulator, Kit tyrosine kinase inhibitor, LanC like protein 2 stimulator, LDL receptor related protein-1 stimulator, Lectin mannose binding protein inhibitor, Leukocyte Ig like receptor A4 modulator, Leukotriene D4 antagonist, Lipoxygenase inhibitor, LOXL2 gene inhibitor, Lysophosphatidate-1 receptor antagonist, Lysophospholipase inhibitor, Macrophage mannose receptor 1 modulator, Macrophage migration inhibitory factor inhibitor, Macrophage migration inhibitory factor modulator, MALT protein 1 inhibitor, Mannan-binding lectin serine protease-2 inhibitor, MAPKAPK2 inhibitor, Matrix extracell phosphoglycoprotein modulator, Matrix metalloprotease inhibitor, MCL1 gene inhibitor, MEKK-5 protein kinase inhibitor, Melanocortin MCi receptor agonist, Melanocortin MC3 receptor agonist, Melanocortin receptor agonist, Membrane copper amine oxidase inhibitor, Metalloprotease-2 inhibitor, Metalloprotease-3 inhibitor, Metalloprotease-9 inhibitor, Metalloproteinase inhibitor-1 inhibitor, Metalloproteinase inhibitor-2 inhibitor, Mineralocorticoid receptor antagonist, Mitochondrial 10 kDa heat shock protein stimulator, MKL myocardin like protein inhibitor, MMP1 gene stimulator, Monocarboxylate transporter inhibitor, Motile sperm domain protein 2 inhibitor, mTOR inhibitor, Mucin stimulator, NACHT LRR PYD domain protein 3 inhibitor, Nek-7 protein kinase inhibitor, Nerve growth factor ligand inhibitor, Neuropilin 2 modulator, Neurotrophin-3 ligand inhibitor, Neurotrophin-4 ligand inhibitor, NGF receptor antagonist, NGF receptor modulator, Nicotinic acetylcholine receptor antagonist, NKT receptor modulator, NKX3.2 gene stimulator, NMDA receptor modulator, Non receptor tyrosine kinase TYK2 antagonist, Nuclear erythroid 2-related factor 2 modulator, Nuclear erythroid 2-related factor 2 stimulator, Nuclear factor kappa B inducing kinase inhibitor, Nuclear factor kappa B inhibitor, Nuclear factor kappa B modulator, Nuclear pore complex protein modulator, Nuclease stimulator, Opioid receptor delta antagonist, Opioid receptor mu agonist, Osteoclast differentiation factor antagonist, Osteoclast differentiation factor ligand inhibitor, Osteoclast immunoglobulin like receptor modulator, OX40 ligand modulator, OX-40 receptor antagonist, Oxidoreductase inhibitor, P2X7 purinoceptor antagonist, p38 MAP kinase alpha inhibitor, p38 MAP kinase inhibitor, p53 tumor suppressor protein stimulator, PDE 3 inhibitor, PDE 4 inhibitor, PDE 4b inhibitor, PDE 5 inhibitor, PDGF receptor antagonist, PGE1 agonist, PGI2 agonist, Phosphoinositide-3 kinase delta inhibitor, Phospholipase A2 inhibitor, Phospholipase C inhibitor, Phospholipase D4 inhibitor, PIM-1 protein kinase inhibitor, PIM-1 protein kinase modulator, PIM-2 protein kinase inhibitor, PIM-3 protein kinase inhibitor, Plasminogen activator inhibitor 1 inhibitor, Plexin domain containing protein stimulator, PPAR gamma agonist, Programmed cell death protein 1 modulator, Programmed cell death protein 1 stimulator, Prostaglandin E synthase inhibitor, Prostaglandin E synthase-1 inhibitor, Protease-activated receptor-2 antagonist, Proteasome beta-8 subunit modulator, Proteasome inhibitor, Protein arginine deiminase II inhibitor, Protein arginine deiminase IV inhibitor, Protein cereblon modulator, Protein kinase C theta inhibitor, Protein kinase inhibitor, Protein MB21D1 modulator, Protein NOV homolog modulator, Protein tyrosine kinase inhibitor, Protein tyrosine phosphatase-2C inhibitor, Proteoglycan 4 inhibitor, Proteoglycan 4 modulator, Proteoglycan 4 stimulator, Proteoglycan stimulator, Proto oncogene vav inhibitor, P-selectin glycoprotein ligand-1, PurH purine biosynthesis protein inhibitor, RAC1 GTPase inhibitor, Retinoid X receptor agonist, Retinoid Z receptor gamma antagonist, Retinoid Z receptor gamma inverse agonist, Rev protein modulator, Rho associated protein kinase 1 inhibitor, Rho associated protein kinase 2 inhibitor, Rhomboid family member 2 inhibitor, RIP-1 kinase inhibitor, RUNX1 gene stimulator, S100 calcium binding protein A4 inhibitor, SCN10A sodium channel subunit inhibitor, SCN9A gene inhibitor, SCN9A sodium channel subunit inhibitor, Secretory phospholipase A2 receptor antagonist, Sepiapterin reductase inhibitor, Seprase inhibitor, Serine threonine protein kinase TBK1 inhibitor, Serine threonine protein kinase TBK1 modulator, Serum amyloid A protein modulator, Signal transducer CD24 modulator, SIRT3 gene stimulator, SL cytokine ligand modulator, SMAD inhibitor, SMAD-2 stimulator, SMAD-3 stimulator, Small inducible cytokine A17 ligand inhibitor, SNAI1 transcription factor inhibitor, Somatostatin 4 receptor agonist, SOX gene stimulator, Sphingosine kinase 1 inhibitor, Sphingosine kinase 2 inhibitor, Sphingosine-1-phosphate receptor-1 agonist, Sphingosine-1-phosphate receptor-1 modulator, Sphingosine-1-phosphate receptor-5 modulator, Sphingosylphosphorylcholine receptor antagonist, Src tyrosine kinase inhibitor, STAT inhibitor, STAT-1 modulator, STAT-3 inhibitor, Stimulator of interferon genes protein inhibitor, Syk tyrosine kinase inhibitor, Synuclein alpha inhibitor, T cell receptor antagonist, T cell receptor modulator, T cell surface glycoprotein CD28 inhibitor, TAKI binding protein modulator, Talin modulator, TAT protein inhibitor, Tau aggregation inhibitor, T-cell antigen CD7 modulator, T-cell differentiation antigen CD6 inhibitor, T-cell surface glycoprotein CD8 inhibitor, T-cell transcription factor NFAT modulator, Tec tyrosine kinase inhibitor, Telomerase stimulator, TGF alpha ligand inhibitor, TGF beta 1 ligand inhibitor, TGF beta 1 ligand modulator, TGF beta 3 ligand inhibitor, TGF beta 3 ligand modulator, TGF beta ligand inhibitor, TGF beta receptor agonist, TGF beta receptor antagonist, TGFB1 gene stimulator, TGF-beta activated kinase-1 inhibitor, TGF-beta type II receptor antagonist, TGF-beta type III receptor antagonist, Thromboxane A2 antagonist, Thromboxane synthetase inhibitor, Thymic stromal lymphopoietin ligand modulator, Thymic stromal lymphopoietin receptor modulator, TLR-4 antagonist, TLR-7 antagonist, TLR-7 modulator, TLR-8 antagonist, TLR-8 modulator, TLR-9 antagonist, TLR-9 modulator, TNF agonist, TNF alpha ligand inhibitor, TNF alpha ligand modulator, TNF antagonist, TNF binding agent, TNF gene modulator, TNF related apoptosis inducing ligand, TRAIL receptor agonist, Transcription factor modulator, Transcription factor p65 modulator, Transcription factor stimulator, Transferase stimulator, Transferrin modulator, Transmembrane 4 L6 family member 19 inhibitor, Transthyretin modulator, TRBV9 gene modulator, TREM receptor 1 antagonist, TrkA receptor antagonist, TRP cation channel A1 inhibitor, TRP cation channel C1 inhibitor, TRP cation channel V1 antagonist, TRP cation channel V1 stimulator, TRP cation channel V4 stimulator, TRPV1 gene inhibitor, Tubulin polymerization protein inhibitor, Tumor necrosis factor 13B receptor modulator, Tumor necrosis factor 13C receptor antagonist, Tumor necrosis factor 13C receptor modulator, Tumor necrosis factor ligand 13 inhibitor, Tumor necrosis factor ligand inhibitor, Tyk2 tyrosine kinase inhibitor, Tyk2 tyrosine kinase modulator, Type I IL-1 receptor antagonist, Type I TNF receptor antagonist, Type II TNF receptor antagonist, Type II TNF receptor modulator, Ubiquitin ligase modulator, Vascular cell adhesion protein 1 antagonist, VEGF receptor modulator, Vimentin inhibitor, Wnt 1 ligand modulator, XPO1 gene modulator, Zap70 tyrosine kinase inhibitor, or a Zinc finger binding protein Aiolos inhibitor.

### Additional Therapeutic Agents for Treating Inflammatory Diseases

In some embodiments, an additional therapeutic agent includes one or more of 608, 610, 611, [11C]-AZ-14132516, [18F]-TZ-Z-09591, [68Ga]Ga-DO3A-S01-GCG, 1,5-bisphenylpyrazole derivative, 101BHG-D01, 11betaHSD1 inhibitors, 19-CP-02, 1-H-11, 2ccPA, 3Sbio, 4P Exenatide, 4P-022, 5-aminolevulinic acid hydrochloride+sodium ferrous citrate, 5-OXO-ETE receptor antagonists, 68Ga-BOT1712, 99mTc-maraciclatide, 9MW-1911, A-1211212, A-1619758, A-2906, A-319, A-4368, AAA-10, AAV8-FGF19 variant M70, AAV-Ex-4, AB-004, AB-03, AB-04, AB-1000, AB-101a, ABA-201, abatacept, ABBV-022, ABBV-3373, ABBV-668, ABBV-712, ABCL-575, ABI-03, Ab-IPL-IL-17, ABIS-45RC, ABM-125, ABP2111-Na, ABP-6016, abrocitinib, ABS-101, ABY-035/AFO2, ABY-062, AC-003, AC-0058, AC-101, AC-201, AC-261066, acarbose, acarbose+metformin, acazicolcept, ACE-1332, ACE-1334, aceclofenac, acetylcysteine, acitretin, aclidinium bromide, aclidinium bromide+formoterol fumarate, acloproxalap, acne, ACQT-1001, ACQT-1127, ACT-101, ACT-777991, actarit, acumapimod, AD-101, AD-17002, AD-201, AD-214, AD-219, AD-222, AD-301, AD-401, adakitug, adalimumab, adalimumab fosimdesonide, adapalene, adapalene+benzoyl peroxide, adapalene+clindamycin hydrochloride, adapalene+clindamycin phosphate, ADB-101, ADB-102, ADC-001, ADD-16, ademetionine, adenosine analogs, aderamastat, Adi, ADI-001, ADi-100, Adipocell, adiponectin agonists, adipose tissue-derived autologous stem cell therapy, adipose tissue-derived mesenchymal stem cell-derived exosomes, adipose-derived stem cell therapy, AD-MSC-CM, ADO-09, AdoShell Islets, ADPO-002, ADPT-02, ADR-001, adrenomedullin, ADS-024, ADS-051, ADSTEM, ADX-097, ADX-246, ADX-248, AER-501, AER-601, aerosolized hydroxychloroquine, afamelanotide, afimetoran, AG-019, AGEX-BAT1, AGM-260, AGMB-129, AGN-242266, AGS-500, AGTX-2003, AGTX-2004, AJ-101, AJ-303, AJM-347, AK-101, AK-119, AK-1830, AKL-4, AKP-08, AKS-107, AKS-440, AL-1, albuterol sulfate, aldafermin, aldesleukin, ALD-R491, ALE-C04, ALE-F02, alefacept, ALF-301, ALF-5755, ALG-055009, alicaforsen, Alitair Pharmaceuticals, ALK5 inhibitor, Allergovac depot, ALLO-329, ALLO-ASC-CD, Allocetra, allogeneic adipose-derived mesenchymal stem cell therapy, allogeneic adult pluripotent stem cells, allogeneic blood-derived CD34-positive adult pluripotent stem cells, allogeneic bone marrow-derived mesenchymal stromal cell therapy, allogeneic bone marrow-derived MSC therapy, allogeneic CAR-iNK cell therapy, allogeneic human islets of Langerhans, allogeneic human pancreatic islets of Langerhans, allogeneic human umbilical cord mesenchymal stem cells, allogeneic mesenchymal stem cell therapy, allogeneic UC-MSC therapy, allogeneic umbilical cord derived mesenchymal stem cell therapy, allogeneic umbilical cord mesenchymal stem cell therapy, allogeneic umbilical cord mesenchymal stem cells, allogenic mesenchymal stromal cell therapy, allogenic umbilical cord-derived mesenchymal stem cell therapy, AlloJoin, AlloNK, AlloRx, alloSTEM-OA, ALM-101, Alma, ALMB-0166, Alnitak program, ALN-KHK, ALN-PNP, alogliptin, alogliptin benzoate+metformin hydrochloride, alogliptin benzoate+metformin hydrochloride XR, alogliptin benzoate+pioglitazone hydrochloride, alpha glucosidase inhibitors, alpha-1 antitrypsin, alpha-4/beta-7 integrin inhibitor, alpha7nAChR agonist, alpha-v/beta-6 integrin inhibitors, alprazolam, ALS-L1023, ALT-100, ALTB-168, ALTB-268, ALTSuLIN, ALY688-SR, AM-1476, AM-411, AM-510, AMA-002, ambroxol hydrochloride, amelenodor, AMG-0101, AMG-329, Amilo-5MER, aminolevulinic acid, aminolevulinic acid hydrochloride, aminopterin, amiselimod, amlitelimab, AMP-activated protein kinase inhibitors, AMPK activators, AMS-III-1086, AMT-101, AMT-126, AMTX-100, AMTX-100 CF, amyloid protein deposition inhibitor, anagliptin, anagliptin+metformin, anakinra, anandamide, Anapsos, ANB-032, ANB-101, anbalcabtagene autoleucel, AND-9, aneratrigine, ANG-4201, anifrolumab, Anima Biotech, annexin V-128, annexuzlimab, antagomiR-144, anti-BMP-2/4 monoclonal antibodies, anti-CD19 CAR T cell therapy, anti-CD19 CAR-NK-cell therapy, anti-CD40 antibody-glucocorticoid conjugate, anti-CD7 CAR T-cell therapy, anti-CXCR3 mAb, anti-EMAP II fully humanized antibodies, anti-F1 gene therapy, anti-fibroblast-activation protein (FAP) antibody radiotracers, anti-fibrotic macrophage cell therapy, anti-IL-1beta/anti-IL-18 bispecific monoclonal antibody, anti-IL-1R7 canonical antibody, anti-IL-23/TL1A therapeutic, anti-IL-4/IL-13 vaccine, anti-MAdCAM-1 CAR T-cell therapy, anti-P2X7 monoclonal antibody humanized, anti-PAD2/anti-PAD4 bispecific humanized mAb, anti-PAR2 therapeutics, anti-TAGE monoclonal antibody, anti-TNF alpha antibody-glucocorticoid conjugate, anti-TNF therapy, anti-TNF-alpha antibody fragment, anti-VEGF-B antibody, Anti-VEGFR therapeutic, Antral, antroquinonol, ANX-009, AP-025, AP-026, apabetalone, APB-R3, APC-201, APD-588, APG-222, APG-333, APG-777, APG-808, APG-990, APGT-001, APH-012, APIRx-1603, APL-10456, APL-1401, APPL-001, apremilast, aprepitant, aprocitentan, APT-101, APX-311, APX-3330, AQ-001S, AQ-280, AQ-312, Aqua Pharmaceuticals, AR-100DP1, AR-110, AR-300, AR-882, ARF6 inhibitors, arformoterol, ARG-201, ARG-301, ARGX-118, ARN-4079, ARO-ALK7, ARO-HSD, ARO-INHBE, ARO-MUC5AC, ARO-PNPLA3, ARO-RAGE, ARO-TSLP, ARQ-234, arsenic trioxide, artenimol, ARTS-011, ARZ-005, ARZC-001, AS-012, AS-100283, AS-1501, ASC-41, ASC-47, asengeprast, asivatrep, ASN-008, ASP-1617, ASP-5502, aspirin trelamine hydrochloride, astegolimab, AT-003, AT-004, AT-005, AT-0287, AT-1001, AT-193, AT-247, AT-278, AT-299, ATA-3219, atacicept, ATB-1013, ATB-1606, Atenas, ATG-201, ATGC-200, ATGC-300A, ATGC-700, ATH-063, ATH-105, ATH-120, ATH-XX (Gene A), ATH-XX (Gene X), ATI-177, ATI-2138, ATL-105, atorvastatin+metformin, ATR-006, ATR-01, ATR-127, ATR-258, Atrosimab, ATTO-002, ATTO-005, ATTO-1310, atuliflapon, ATX-006, ATX-304, AUR-101, AUR-200, auremolimab, autologous adipose tissue-derived mesenchymal stem cells, autologous adipose-derived mesenchymal stem cells, autologous bone marrow mononuclear cells, autologous leukocyte cell therapy, autologous tolerogenic dendritic cell therapy, autologous tolerogenic dendritic cell vaccine, autologous T-reg cell therapy, autoSTEM-OA, AUX-101, avacopan, AVB-114, AVD-1001, avenciguat, AVI-3307, AVID-200, AVM-0703, AVO-101, AVO-1681, AVT-001, AVT-16, AVX-001, AWEPO-003, AX-0601, AX-0810, AX-158, AX-202, AX-2911, AXA-1125, AXR-2102, AZD-0284, AZD-0449, AZD-1163, AZD-1656, AZD-1705, AZD-2389, AZD-2693, AZD-6912, AZD-7798, AZD-8630, AZD-9550, azelaic acid, azelastine, azemiglitazone potassium, azithromycin, B-1344, B-1654, B-244, Bacillus licheniformis, Bacmune, bacterial infection), baicalin+catechin, balsalazide, balsalazide sodium, bambuterol, baricitinib, barzolvolimab, basal insulin, BAT-6026, BAY-1830839, bazlitoran, BB-1511, BB-265, BBACN, BBI-03, BBI-6000, BBT-401, BBT-401-IS, BBT-877, BC-0306, BCD-180, BCG polysaccharide+nucleic acid, BCI-332, BCKDK inhibitor, BCMA-CD19 cCAR, BEBT-503, BEBT-508, beclometasone dipropionate+formoterol fumarate, beclomethasone dipropionate, beclomethasone dipropionate+formoterol fumarate+glycopyrronium bromide, bedoradrine, begelomab, beinaglutide, belantamab, belapectin, belatacept, belimumab, belumosudil, bempedoic acid, bempikibart, BEN-8744, bencycloquidium bromide, benralizumab, benzoyl peroxide, benzoyl peroxide+tretinoin, berberine+ursodeoxycholate, berberine chloride, berberine ursodeoxycholate, berdazimer sodium, bermekimab, bersacapavir, bersiporocin dihydrochloride, bertilimumab, betamethasone, betamethasone dipropionate, betamethasone valerate, bexagliflozin, bexotegrast, Bezafibrate, bezafibrate+obeticholic acid, BF-114, BFP-002, BFP-102, BG-148, BG-2102, BGB-23339, BGM-0504, BGP-014, BGS-1949, BGT-002, BGT-004, BG-T19, BHD-1028, BHV-1300, BI-1291583, BI-1323495, BI-3006337, BI-3231, BI-655064, BI-706039, BI-706321, BI-765250, BI-765423, Bifunctional complement inhibitor, BIIB-068, BIIB-110, bilastine, bimekizumab, bimiralisib, BIO-11006 Inhalation Solution, BioChaperone AsPram, BioChaperone Combo, BioLexa, biosimilar monoclonal antibodies, BIOT-001, bispecific KIR/ICOS antibody CD8 Treg modulator, BITT-CD4D11, BITT-CD4F10, BL-001, BLD-2660, BLD-3051, BLI-1006, BLI-1007, BLR-200, BLU-808, BLX-0631, BMC-333, BMN-255, BMS-336, BMS-687681, BMS-820132, BMS-986142, BMS-986313, BMS-986322, BMS-986325, BMS-986326, BMX-010, BNC-1602, bone-marrow derived allogenic ischemia-tolerant MSCs, boningmycin, BOS-161721, BOS-475, BOS-580, Bosakitug, bosentan, BOT-1712-IFNg-SD, bovhyaluronidase azoximer, bovine lactoferrin, Box-5, BP-012, BP-103, BPYT-01, BR-1019, BR-201, BR-3003, BR-3005, branched-chained aminotransferase-1 inhibitors, branebrutinib, BRD4 inhibitors, BRE-AD01, brensocatib, brentuximab vedotin, brepocitinib, brilacidin, brilaroxazine hydrochloride, briquilimab, BRL-186616, BRL-191118, BRL-201009, BRL-301, BRM-521, brodalumab, bromocriptine, BRS-201, BS-1801, BSI-056T, BSI-502, BTI-320, BTX-1204, BTX-1308, BTX-1503, budesonide, budesonide+arformoterol, budesonide+formoterol, budesonide+formoterol fumarate, budesonide+procaterol hydrochloride, budesonide+salbutamol, budesonide+salmeterol, budoprutug, buloxibutid, burfiralimab, BV-200 series, BVX-20, BX-002, BX-003, BXT-102, BX-U001, BX-U004, BZ-068, BZ-371, BZ-371B, C-455, C4X-6746, C-867, CA-508, CA-702, CABA-201, cadisegliatin, cagrilintide, cagrilintide+semaglutide, CAL-4, calcipotriol, calcipotriol+betamethasone, calcipotriol+betamethasone dipropionate, calcipotriol+cortisone, calcitriol, CALY-002, camoteskimab, CAN-10, canagliflozin, canagliflozin+metformin IR, canagliflozin+metformin XR, canakinumab, cannabidiol, cannabidiol+dronabinol, cannabidiol+tetrahydrocannabinol, cannabinoid, cannabinoid 1 receptor antagonists, cannabinoid CB2 receptor agonist antibody, cannabinoid/celecoxib conjugate, capeserod, capsaicin, CAR Treg-cell therapy, carbon dioxide+perfluorooctyl bromide, CARD9/TRIM62 interaction inhibitors, carotegrast methyl, CartiLife-Allo, Cartistem, cavosonstat, CB-010, CB-01-35, CB-0406, CB-06-01, CB2 receptor agonists, CB-4211, CB5138-3, Cbi-Ex4, CBL-514, CBM-588, CBW-511, CC-90001, CC-90006, CC-92252, CC-97540, C-CAR168, CCI-15106, CCRL2 modulators, CCX-507, CCX-624, CCX-872, CD19-BCMA CAR-T, CD19-CAR-DNT, CD24-IgFc, CD4 antigen modulator/CD8 antigen modulator/T cell receptor modulator, CD4+CD127lo/−CD25+polyclonal regulatory T cells, CDL-200, CEE-321, CEL-383, CEL-4000, celastrol, celecoxib, celecoxib+tramadol hydrochloride, cell therapy, cell-free stem cell-derived extract formulation, Cellgram-LC, Cellistem-OA, CELZ-001, CELZ-100, CELZ-101, CELZ-201, cendakimab, cenerimod, cenicriviroc, certolizumab pegol, CG-459, CG-652, CGT-8012, Chanllergen, CHF-6333, CHF-6366, CHF-6550, chiglitazar, CHIN-117, CHM-273S, Chondrocell-Ex, chromene COX-2 inhibitors, cibinetide, ciclesonide, ciclosporin, CIDEB siRNA therapy, CIGB-258, cilofexor+firsocostat, cilofexor tromethamine, CIN-244, ciprofloxacin hydrochloride, CIT-013, citric acid monohydrate, CJC-1134-PC, CJRB-203, CJRB-204, CJRB-402, CJRB-901, CK-0045, CKBA, CKD-371, CKD-378, CKD-379, CKD-383, CKD-386, CKD-389, CKD-393, CKD-398, CKD-506, clarithromycin, clascoterone, class I HDAC inhibitors, CLB-004, CLBS-03, clesacostat+ervogastat, CLF-065, clindamycin, clindamycin phosphate+benzoyl peroxide, clindamycin phosphate+tretinoin, CLM-022, CLN-978, clobetasol propionate, clobetasol propionate+tretinoin, clodronic acid, Clostridium butyricum, CM-101, CM-272, CM-326, CM-512, CMK-389, CMR-316, CMS-D001, CNP-103, CNP-104, CNTX-6970, CNTY-101, cofrogliptin, colchicine, colesevelam, ColiFin, collagen I translation inhibitors (liver fibrosis), COPD vaccine, cord blood derived stem cells, corticotropin, corticotropin releasing factor receptor-2 inhibitors, COYA-203, COYA-204, CP-101, CPL-207-280, CPL-302415, CPL-409116, CRB-0017, CRB-486, crisaborole, Crohn's disease therapeutic, crovalimab, CS-0159, CS-060380, CS-090537, CS-120070, CS-12192, CS-17919, CS-20AT04, CS-27109, CS-32582, CS-43001, CSA-13, CSJ-117, CSPCHA-115, CT-02, CT-05, CT-15300, CT-303, CT-388, CT-732, CT-868, CT-996, CTI-156, CTI-168, CT-P17, CT-P55, CTX-101, CTX-112, CTX-211, CTXT-102, CU-06, CU-10101, CU-104, CUD-005, cudetaxestat sodium, CUE-301, CUG-252, CUR-N399, Cutaquig, CVB1 vaccine, CVI-301, CVL-001, CVXL-0074, CX-011, CX-1440, CXCL9/CXCL10/CXCL11 gene therapy, CXCR5 targeting antibody, CXF-11, CXG-86, CXG-87, CYP-004, cyproterone acetate+ethinyl estradiol, CYRS-101, CYRS-MD02, Cyt-108, CYTX-100, D-2570, D4-103-01, D4-103-02, D4-103-03, D4-103-04, D4-107-01, D4-201-01, D-745, D-759, DA-1241, DA-1726, DA-302168S, DA-5202, DA-5221-T, dalpiciclib, daniluromer, danuglipron, dapagliflozin+glimepiride+metformin hydrochloride, dapagliflozin+linagliptin, dapagliflozin+metformin, dapagliflozin+sitagliptin, dapagliflozin+sitagliptin+metformin hydrochloride, dapagliflozin+teneligliptin, dapagliflozin+vildagliptin, dapagliflozin citrate, dapagliflozin ER, dapagliflozin propanediol, dapagliflozin propanediol+sitagliptin phosphate, dapansutrile, dapirolizumab pegol, dapsone, daratumumab, daridorexant hydrochloride, daxdilimab, dazodalibep, dazukibart, DB-007-4, DB-2304, DBI-001, DBI-500, DBM-1152A, DC-291407, DC-806, DC-853, DCR-CLD, DCR-LIV2, DD-01, DD-02, DD-03, DD-15, deflazacort, delgocitinib, DEN-181, denatonium acetate monohydrate, dendritic cell-based microsphere vaccine, denifanstat, denosumab, depemokimab, dersimelagon, Des-1 inhibitor, Descartes-08, desloratadine, desogestrel+ethinylestradiol, desonide, deucravacitinib, deuruxolitinib phosphate, dexamethasone, dexamethasone sodium phosphate, dexlansoprazole, dexpramipexole, DFL-23806, DFV-890, DiabeCell, diacerein, Diamyd, diclofenac, diclofenac etalhyaluronate sodium, diclofenac sodium, difamilast, dimethyl fumarate, dimethyl fumarate+ethyl hydrogen fumarate calcium+ethyl hydrogen fumarate magnesium+ethyl hydrogen fumarate zinc, diroleuton, dithranol cream, divozilimab, DJ-X-013, DKM-412, DKM-420, DL-0309, DLA bispecific antibody program, DLBS-3233, DLQ-02, DLX-105, DLX-2323, DM-1050, D-methyldopa, DMT-210, DMT-310, DMX-700, DMXD-011, DNA-encoded anti-IL23 monoclonal antibody, DNX-114, DNX-314, DONQ-52, dornase alfa, dorzagliatin, doxofylline, doxofylline (bronchiectasis), doxycycline hyclate, doxycycline hyclate (delayed release), doxycycline hyclate (easy-to-swallow, DPT-0218, DR-10624, Drone Treg, drospirenone+ethinylestradiol, DS-7011, DT-109, dual agonist combination 1 therapy, dual agonist combination 3, dual alpha-V/beta-1 and alpha-5/beta-1 integrin inhibitors, dual AMCase/CHIT1 inhibitors, dual anti-CD19/anti-BAFF CAR T-cell therapy, dual GLP1/glucagon receptor agonists, dual inhibitors of sphingosine kinase 1 and 2 (SPHK1/2) University, dual JAK3/TEC inhibitor, dual PPAR-alpha/gamma agonists, dual PPAR-gamma/delta modulators, Duglow, dulaglutide, duloxetine, dupilumab, dust mite vaccine, DW-2007, DW-2008S, DWC-202001, DWC-202002, DWC-202213, DWJ-1525, DWJ-1563, DWP-213388, DWP-457, DWRX-5003, DX-819511, DYRK inhibitor, DYV-024, DZ-2002, ElK, E40-02, E-6742, EA-1080, EA-3571, EB-005, EB-06, EBI-H, eblasakimab, EBX-102, ECC-0509, ECC-4703, ECC-5004, eclitasertib, ecnoglutide, EcoActive, eculizumab, edaravone, edecesertib, edralbrutinib, efavaleukin alfa, efgartigimod alfa, efinopegdutide, eflomithine, efpeglenatide, efruxifermin, efzofitimod, EG-501, EI-001, eicosapentaenoic acid, elafibranor, elapegademase, elarekibep, elebsiran, Elixcyte, ELN-201, ELN-27, elobixibat, elsubrutinib, eltanexor, EM-101, emapalumab, emedastine, EMI-137, emiplacel, empagliflozin, empagliflozin+linagliptin, empagliflozin+linagliptin+metformin XR, empagliflozin+metformin, empagliflozin+metformin extended-release, empasiprubart, EN-2000, ENA-002, enavogliflozin, ENB-104, ENB-105, ENB-106, ENB-109, ENB-110e, ENC-0364, ENC-0653, ENC-201, encapsulated Melligen cell therapy, endonuclease modulators, enekinragene inzadenovec, ENERGI-F702, ENERGI-F704, ENERGI-F708, engineered regulatory T cell therapy, ENN-0403, enpatoran, ensifentrine, ensifentrine+glycopyrrolate, ENT-001, enteric-coated mycophenolate sodium, Enterome, EOM-613, EP-104-GI, EP-262, EP-9001A, EPD-401, epeleuton, EPGN-696, Epi-13, epinastine hydrochloride, epinephrine, EpiTight, EPM-301, eptotermin alfa, EQ-101, EQ-121, EQ-302, ER-000145, erdosteine, erlotinib, ERp44-adiponectin interactions modulators, ertugliflozin, ertugliflozin+metformin, ertugliflozin+sitagliptin, ervogastat, esflurbiprofen patch, ESK-001, esomeprazole, esomeprazole sodium, ET-901, etanercept, EtanerRel, ETD-001, ETH-47, etodolac, etoricoxib, etrasimod, etrinabdione, ETX-291, ETX-312, EV-104b, evexomostat, evogliptin, evogliptin+dalpagliflozin+metformin, evogliptin+metformin XR, EVT-401, EVX-B4, Exd-391209, EXE-346, exenatide, exendin-4-Fc fusion protein, exendin-4-human serum albumin fusion protein, EXG-34217, EXL-01, ExoFlo, exosome based therapeutic, extended release glipizide, EYD-001, EYP-002, ezurpimtrostat, F-200, F-528, F-573, factor D inhibitor, farnesoid X receptor agonists, farudodstat, fazirsiran sodium, FB-102, FB-1603, FB-1807, FB-401, FB-704A, FB-825, FB-918, FBL-MTX, FBPase inhibitors, FCR-001, FCX-013, FE-999322, FE-999324, felzartamab, fenebrutinib, fenofibrate+atorvastatin, fepixnebart, fevipiprant, fexuprazan, fezagepras, FFAR4 activator program, FFAR4 agonists, FG-3149, FGF21 gene therapy, FG-M701, FIA-586, FIB-918, FIB-992, filgotinib maleate, FIN-524, FIN-525, fipaxalparant, firsocostat, FKC-288, fluasterone, flunisolide, fluocinonide, fluorofenidone, fluticasone, fluticasone+formoterol, fluticasone furoate, fluticasone furoate+umeclidinium+vilanterol, fluticasone furoate+vilanterol trifenatate, fluticasone propionate, fluticasone propionate+formoterol fumarate, fluticasone propionate+salbutamol sulfate, fluticasone propionate+salmeterol, fluticasone propionate+salmeterol xinafoate, FM-101, FNS-007, foralumab, forigerimod, formoterol, formoterol fumarate, formoterol fumarate+fluticasone propionate, formoterol fumarate+glycopyrronium bromide, foscenvivint, fostamatinib disodium, fotagliptin benzoate, FOXO1 inhibitor program, FP10.47, FPP-003, FPP-005, fresolimumab, frexalimab, froniglutide, FRTX-02, FSI-965, FT-4101, FT-819, FTC-001, Fuzheng Huayu capsule, FW-420, FW-424, FWB-1313, FXR agonists, FXR-314, FZ-006, FZ-007, FZ-020, FZJ-003, G-001, G2GBio, G3PP activator program, G-49, GABA+antigen based therapy, GABAA receptor agonists, gabapentin, GAD-65, Gal-300, galectin-3 inhibitors, gallium (68Ga) rofapitide tetraxetan, GalNAc-conjugated siRNA therapeutic, Gamunex, GARV-AAV2-A20, GB-001, GB-004, GB-0895, GB-12, GB-19, GB-20, GB-5313, GB-7001, GB-910, GB-930, GB-G01, GC-012, GD-134, GDD-3898, GD-iExo-001, GDNF, gefurulimab, gemigliptin+dapagliflozin, gemigliptin+metformin HCl, gemigliptin+rosuvastatin, gemigliptin tartaric acid, GEN-1503PR, GEN-501, Gensci-048, GF-1002, GF-103, GF9-LPC loaded TREM-1 receptor inhibitor, GFH-312, GH-509, GI-210, GKAC, GL-0034, GL-7190, glargine biosimilar, glibenclamide, gliclazide, glimepiride, glimepiride+extended-release metformin hydrochloride, glimepiride+metformin, glimepiride+vildagliptin+metformin, GLP-1 analog-COL3A1 fusion protein, GLP-1 analogs, GLP-1 receptor agonist, GLP-1/exendin-4-Fc fusion proteins, GLP-1/GIP receptor agonist, GLP-1-Fc-PYY, GLP-2, GLPG-3667, GLPG-4876, glucagon-like peptide-1 analog, glucose responsive smart basal insulin, glutathione+ascorbic acid+bicarbonate, glutazumab, GLY-200, glycopyrrolate+formoterol fumarate+budesonide, glycopyrronium+formoterol fumarate+fluticasone propionate, glycopyrronium+vilanterol, glycopyrronium bromide, glycopyrronium bromide+indacaterol maleate, GLY-POL, GM-60106, GM-90194, GMA-106, GMA-107, GMA-10X, GMA-10Y, GMDP, GM-XANTHO, GN-037, GNF-2133, GNKS-356, GNR-068, GNR-086, GNR-087, GNS-3595, GNSC-001, golexanolone, golidocitinib, golimumab, gosogliptin, GPCR antagonists, GPR15 antagonists, GPR35 agonist, GPR35 inhibitor, GPR39 antagonists, G-protein-coupled receptor modulator, GPX-002, GPX-003, GQ-501, GR-010, GR-012, GR-018, GR-019, GR-1501, GR-1603, GR-1802, GR-2002, GR-2301, GR-2303, grapiprant, grapiprant (iv), Grastek, GRC-39815, GRI-0124, GRI-0803, Grp94 inhibitors, GRT-6015, GS-0151, GS-0272, GS-1069518, GS-1427, GS-300, GS-400, GS-6791, GS-834356, GSBR-1290, GSK-1070806, GSK-2276186, GSK-2618960, GSK-2646264, GSK-2831781, GSK-2982772, GSK-3117391, GSK-3179106, GSK-3858279, GSK-3862995B, GSK-3923868, GSK-4347859, GST-HG-151, GT-1908, GT-20029, GT-TNF alpha-mAb 004, GTX-011, gumokimab, gusacitinib, guselkumab, GUT-103, GUT-108, gut-restricted G-protein coupled bile acid receptor 1 agonist, gut-restricted TGF-beta receptor 1 kinase inhibitor, GV-101, GXCPC-1, GX-G6, GXHPC-1, GXIPC-1, GZ-21T, GZR-101, GZR-18, GZR-33, GZR-4, H-008, H-018, halobetasol propionate, halobetasol propionate+tazarotene, halogenated xanthene, halometasone, HB00-17, HB-0034, HB-0043, HB-1085, HB-1734, HB-401, HB-601, HBM-9001, HBM-9378, HBP-01, HCL-001, HCW-9302, HD-1916, HD-7671, HDM-1002, HDM-3010, HDNO-1605, HE-009, HEC-192334, HEC-44616, HEC-88473, HEC-96719, HECB-1800301, Hemay-007, HEMP-001, HepaStem, heterologous liver cell therapy, HFG-201, HGR-4113, HHT-109, HI-1640V, histamine human immunoglobulin, Hizentra, HJ-178, HJ-787, HJC-0416, HK-1, HK-660S, HL-012MA, HL-08, HL-231, HL-300, HLA-DQ2/DQ8 inhibitors, HLA-DR1/DR4 inhibitors, HLA-open conformer-specific monoclonal antibody, HLK-6002, HM-002-1005, HM-12460A, HM-15136+efpeglenatide, HM-15211, HM-15275, HM-201, HMBD-011, HNF4A mRNA therapeutics, house dust mite allergen, house dust mites immunotherapy, HP-1901, HP-515, Hpb glutamate dehydrogenase modulator, HPD-001, HPG-1860, HPG-5119, HPG-7233, HPN-01, HPN-FO1, HPP-3033, HPP-737, HpVac-R13, HR-17031, HR-19042, HR-20033, HRF-2105, HRG-2005, HRS-5965, Hrs-7085, HRS-7535, HRS-9531, HRS-9821, HRX-0215, HRX-0701, HS-101, HS-10356, HS-10374, HS-10501, HS-20004, HS-20094, HS-401, HSD17B13 gene therapy, HSD17B13 inhibitors, HSD17b13i, HSK-31679, HSK-34890, HSK-39004, HSK-39297, HT-004, HT-201, HT-202, HTL-0027477, HTL-0033744, HTL-30023, HTPEP-002, HU-6, HuL-001, human adipose-derived mesenchymal stem cells, human albumin, human amniotic exosomes, human FGF-1, human insulin biosimilar, human plasma gelsolin, human pluripotent embryonic stem cell-derived pancreatic islet cells, human pluripotent stem cells derived allogenic hepatocytes, human umbilical cord-derived mesenchymal stem cell therapy, human-derived sertoli cell therapy, humanin analogs, HWH-486, HX-100101-1, HX-1171, HY-021068, HY-071221, HY-07170702, HY-072808, HY-1770, HY-1839, HY-209, hyaluronate sodium, hyaluronic acid, HYBR-011, hydrogel-exenatide, hydronidone, hydroxychloroquine, hydroxyl dendrimer-alendronate conjugate, hymecromone, hypericin, hypochlorous acid, hypoglycemic agents, HZ-010, HZ-012, HZBio-2, HZ-J001, I-039, i2o-107, i2o-110, i2o-120, IA-14069, ianalumab, IAPP inhibitor, IBD-3540, IBD-98-M, iberdomide, IBI-3002, IBI-355, IBI-356, IBIO-100, IBL-101, IBP-9414, ibrigampar, ibuprofenamine hydrochloride, Ibutamoren, icanbelimod, IcBD-01, ICM-203, ICM-203-0, icomidocholic acid, icosabutate, icovamenib, ICP-332, ICP-488, ICP-490, iCP-NI, ICT-21, ID-110521156, ID-11903, ID-119050134, IDG-16177, IDL-2965, IDO-8, ifetroban, IFNalpha kinoide, IFN-ELP fusion protein, IgE inhibitors, iguratimod, IHL-675A, Ii-key/MHC class II epitope hybrid peptide immunomodulator peptide vaccines, IK-01, IL-10 INDUKINE, IL-10 NanoCap, IL-1512, IL-17 NanoAb, IL-2 fusion protein, IL-23R inhibitor, IL-23R-CAR-Treg cell therapy, IL-25 targeted therapeutic, IL-4R alpha antagonist, IL-4Ra targeted therapeutic, IL-6 inhibitors, ilaprazole, ILB-2107, iloprost, ILP-100, ILV-001, IM-102, IMB-002, IMB-1001, IMB-1003, IMB-1004, IMB-101, IMC-S118AI, IMCY-0098, imeglimin, IMG-004, IMG-007, IMG-008, IMG-036, IMG-1, imidazole salicylate, IMM-124-E, IMM-H014, ImmTOR+PDC-E2, immune globulin intravenous, Imotope, IMP-761, implantable allogeneic chondrocyte cell therapy, IMPT-514, imrecoxib, IMS-001, imsidolimab, IMT-001, IMU-856, imvotamab, IMVT-1402, IMX-120, IN-A002, IN-A004, IN-A010, inaticabtagene autoleucel, IN-B012, IN-B013, INCB-054707, indacaterol, indacaterol acetate+glycopyrronium bromide+mometasone furoate, Indamet, inebilizumab, INF-108, infliximab, ingestible enteric oxygen monotherapy, INI-822, INO-002, INS-068, insluin lispro, insparin, insulin, insulin aspart, insulin degludec, insulin degludec+insulin aspart, insulin degludec+liraglutide, insulin degrading enzyme inhibitors, insulin efsitora alfa, insulin glargine, insulin glargine+lixisenatide, insulin icodec, insulin lispro, insulin lispro+exenatide, insulin lispro+pramlintide, insulin mouth rinse, insulin tregopil, insulin-producing stem-islet beta cell hybrids, Insuman Implantable, INT-220, INT-787, Integrin alpha-2/beta-1 inhibitor, integrin alpha-4/beta-7 antagonists, Integrin alpha-5/beta-1 inhibitor, interferon alfa-2a follow-on biologic, interferon alfa-n1, interferon gamma follow-on biologic, interferon-alpha, Interleukin IL-17A inhibitor, interleukin-2 follow-on biologic, INV-002, INV-007, INV-008, INV-101, INV-103, INV-17, INVA-8001, INVA-8002, invopressin, IOA-289, ION-224, ION-769357, IONIS-GCGRRx, IOT-022, IPG-008, IPG-011, IPG-1094, IPG-11406, IPG-7236, IPN-60250, ipragliflozin, ipragliflozin+sitagliptin, ipratropium+fenoterol, ipratropium bromide, ipratropium bromide+salbutamol sulfate, iPSC cell therapy, iPSC-derived pancreatic islet cell therapy, iPSCs derived cell therapy, iptacopan, IQ-004, IR-444, IRElalpha inhibitors, IRL-201104, IRL-201805, IRX-4204, iscalimab, ISD-017, ISM-001, ISM012-042, isotretinoin, itacitinib, ITCA-1601, itepekimab, iTOL-101, iTOL-102, itolizumab, ITV-1, ivacaftor, ivarmacitinib sulfate, ivarmacitinib sulfate (SHR-0302), IVB-001, ivermectin, IVL-4001, ivonescimab, iXB-401, IxCell hUC-MSC-O, ixekizumab, izencitinib, izokibep, izumerogant, J2H-1702, JadiCell therapy, JAG-301, JAK inhibitors, JAK2 inhibitors, JAK-989, jaktinib dihydrochloride monohydrate, jaktinib hydrochloride, JBI-1044, JH-013, JK-0001, JK-0002, JK-0003, JKN-24011, JMJD3 siRNA, JMKX-000189, JMT-202, JMT-601, JNJ-1459, JNJ-2113, JNJ-3534, JNJ-67484703, JNJ-78934804, JNJ-9280, JP-1366, JP-2266, JRF-106, JRF-401, JRP-878, JS-005, JT-003, JTA-004, JTE-051, JTE-451, JTT-251, JTT-662, JW-0201, JW-1601, JW-202232, JW-202313, JW-2202, JYB-1904, JYP-0061, JYP-0066, K(D)PT, K-1032, K-11, K-757, K-833, Ka Shu Ning, kamuvudines, KAN-002, KAN-101, KB-5XX, KBL-382, KBL-693, KBL-697, KBL-982, KBLP-004, KBLP-007, KBP-336, KBP-3571, KD-4002, KD-4004, KD-6005, KEM-2102, KEM-2104, ketoprofen+omeprazole, KF-0210, KF-041, KGYY-15, KH-629, KH-656, KH-805, KH-806, KH-917, KI-696, KINE-201, KIO-101, KITCL-27, KK-4277, KL-130008, KN-002, KN-056, KN-5501, KP-104, KP-470, KPG-818, KPI-012, KPL-404, KQ-791, KSHB-005, KSP-0243, KT-294, KT-474, KT-621, KT-A112, KT-A522, KT-A832, Kvl.3 ion channel mAbs, KX-826, KY-41111, KYLO-0603, KYS-202002A, KYV-101, KYV-201, L-47, L-608, LABA+LAMA therapy, LABP-111, LABP-69, LABthera-006, Lactobacillus exosomes, ladarixin, LAE-103, LAE-104, LAE-105, LAE-106, laflunimus, Langopept, lanifibranor, lansoprazole, lapretolimod, larsucosterol, LAS-200019, LAT-8881, latiglutenase, LB-600, LBG-1600M, LBP-02, LB-P7, LB-P8, LBS-009, LBSA-0103, LC-542019, LCK inhibitor, LD-09163, LD-2020, lebrikizumab, leflunomide, LEM-S402, lenabasum, lepzacitinib, leramistat, leronlimab, leucine+metformin+sildenafil, levalbuterol, levalbuterol hydrochloride, LEVI-04, levilimab, levonorgestrel+ethinylestradiol, LG-00034053, LG-203003, LG-283, L-glutamine, LGM-1506, LGM-2605, LH-021, LH-1801, LH-8, licogliflozin bis(prolinate), linagliptin, linagliptin+dapagliflozin+metformin hydrochloride, linagliptin+metformin hydrochloride, linagliptin+metformin XR, linerixibat, LipoCartigen, liraglutide, lisaftoclax, LIT-00505, lithium succinate, litifilimab, LIV-001, liver disease/non-alcoholic steatohepatitis therapy, lixisenatide, LLDT-8, LM-011, L-methyldopa, LMT-503, LMY-920, LN-3118, LNA-043, LNK-01001, LNK-01003, LNK-01004, LNP-1955, LNP-CDP mRNA therapy, LNR-653.1, lobeglitazone+metformin, lobeglitazone+sitagliptin, LOFC-00501, Lomecel-B, londamocitinib, long acting beta agonist/long acting muscarinic agonist, long-acting aerosolized peptide-based therapy, lonodelestat acetate, lorecivivint, low dose human recombinant interleukin-2, 1p-003, LP-0200, LP-168, LP-342, LPCN-1148, LPS-001, LPXT-007, LQ-036, LQ-041, LQ-043, LR-1, LR-19018, LR-19019, LR-19024, LR-19025, LR-19131, LR-20016, LR-20022, LR-21004, LRH-1 modulators, LrS-235, LRX-712, LS-301, LT-002-158, lucinactant, lumiracoxib, lumrotatug, LUNAR-TKD, lunsekimig, luseogliflozin hydrate, lusvertikimab, LUT-014, lutikizumab, LW-104, LXR inverse agonists, LY-3493269, LY-3509754, LY-3522348, LY-3532226, LY-3537021, LY-3549492, LY-3556050, LY-3849891, LY-3857210, LY-3871801, LY-3872386, LY-3885125, LY-3938577, LY-3972406, LY900027, LYS-006, lysophospholipase inhibitor, LZM-012, M-002, M-008, M-119102, M-127101, M3 muscarinic receptor antagonists, M-43, M-605110, M-610101, MaaT-033, macitentan, MAdCAM targeted DK4/10, manfidokimab, MAP-315, maralixibat chloride, maraviroc, MAS-825, masitinib, masoprocol, MAX-40070, maxacalcitol, maxacalcitol+betamethasone, Mayne, mazdutide, MB-001, MB-1, MB-204, MB-310, MB-CART19.1, MBF-118, MB-N-008, MBQ-167, MBRI-003, MB-XO1Y03, mCLC-846, MCM-001, MDI-1228, MDI-2517, MDNA-413, MDPK-67b, ME-001, ME-003, ME-3183, mecasermin, Medac Pharma, MEDI-0618, MEDI-7352, Melgain, meloxicam, memantine derivatives, mepolizumab, mesalamine, mesalazine, Mesenchymal stem/stromal cell therapy, mesenchymoangioblast-derived mesenchymal stem cell therapy, MET-2, MET-409, MET-642, metenkefalin acetate+tridecactide acetate, metformin, metformin+glibenclamide, metformin glycinate, metformin hydrochloride, metformin XR+valsartan+atorvastatin, methotrexate, methotrexate (sc, methyl aminolevulinate hydrochloride, methylprednisolone, methylprednisolone hemisuccinate, methylprednisolone suleptanate, MFC-0101, MFC-0102, MG-01, MG-K10, MG-S-2525, MGY-1838, MG-ZG122, MH-002, MH-004, MH-080, MHS-552, MHV-370, mibavademab, MIC-Lx, microbiome modulator, microbiome-based live bacterial probiotic, microparticle vaccine, midismase, miglitol, MIIST-305, MIL-62, milk-fermented probiotic product, minocycline, minocycline+adapalene, minocycline hydrochloride, miR-132 antisense oligonucleotide inhibitor, miricorilant, mirikizumab, MI-S4, misoprostol+diclofenac, MIT-001, mitiglinide, mitiglinide+metformin, mitiglinide+voglibose, mitiperstat, Mitizax, mitochondrial uncouplers, MIV-711, mizoribine, MK-1092, MK-3655, MK-6204, MK-7480, MKP-10241, MLX-0800, MLX-5000, MLX-7000, MM-09, MM-II, MNO-863, momelotinib, mometasone, mometasone+formoterol, mometasone furoate, mometasone furoate+indacaterol acetate, monlunabant, montelukast, montelukast sodium, montelukast sodium+levocetirizine dihydrochloride, MORF-057, MORF-627, mosedipimod, mosunetuzumab, mouse monoclonal antibody against human interleukin-8, MP-1032, MP-2021, MPB-2354, MPC-300-IV, MPO-0144, MRG-229, mRNA encoding runt-related transcription factor-1, mRNA-6981, mRNA-LNPs expressing EGF and HGF, MRT-6160, MS-001, MSB-01, MSB-03, MSB-3163, MSM-605, MT-1002, MT-105, MT-2002, MT-2004, MT-5562, MT-971, MTC-896, MT-SYK-03, MTX-101, MTX-463, MTX-474, mucosa-associated lymphoid tissue lymphoma translocation protein 1 inhibitors, mufemilast, MufroSyn, mugwort pollen allergen vaccine, muscarinic M3 receptor antagonist, MWN-101, MWN-102, MY-004, MY-009212A, mycophenolate mofetil, Myeloid activating target, MYJ-1633, MYMD-1, myonectin, N-65 lacritin, NA-941, nabumetone, nacystelyn, nadifloxacin, nadolol, nalfurafine, naltrexone, namodenoson, nanofitins, NAP-1051, naproxen+esomeprazole, naproxen+esomeprazole strontium, narsoplimab, natalizumab, nateglinide, nateglinide+metformin hydrochloride, NAV-240, Navacims, NBL-012, NC-01, NC-101, NCP-111, NCP-112, NCR-100, ND-003, NDC-0009, NDX-3315, NDX-3324, necroptosis inhibitors, nedocromil, nemolizumab, NEO-TRA1, nerigliatin, netakimab, netazepide, NeuLiv, neuropeptide Y2-receptor agonists, NEXI-005, next generation peptidomimetic and tissue transglutaminase inhibitors, next generation smart glucose-responsive insulin, NF-2107, NGM-395, NGRM-001, NI-203, nibrozetone, niclosamide, nicotinamide N-methyltransferase inhibitor, NIK inhibitors, NIM-1324, nimotuzumab, ninerafaxstat trihydrochloride monohydrate, NIP-046, NIP-322, NIPEP-CARE, nipocalimab, nisotirostide, nitazoxanide, nitric oxide, nitroglycerin, NJA-005, NKTT-320, NKX-019, NLP-91, NLRP3 gene therapy, NLRP3 inflammasome inhibitor, NM26-2198, NMX-2, NN-1471, NN-1535, NN-1845, NN-6177, NN-6561, NN-6581, NN-6582, NN-9041, NN-9541, NNC-0113-6856, NNC0113-6860, NNC0113-6861, NNC0113-6891, NNC-0194-0499, NNC-0472-0147, NNC0480-0389, NNC-0519-0130, NNC-0650-0013, NNC6022-0001, NNC-965, nomacopan, non-alcoholic fatty liver disease agent, norethindrone acetate+ethinylestradiol, norucholic acid, noscapine/noscapine analogs, NovDB2, NovFS, NOX inhibitors, NOX4 inhibitors, NP-011, NP-339, nrf2 activator, NS-402, NS-403, NT-0004, NT-0007, NT-0008, NTCP inhibitor, NTM-006, NTR-441, NTX-748, nucleoside-modified TERT mRNA, NV-422, NV-556, NVS-451, NX-13, NX-5948, NX-73, NX-9001, NYX-2925, 0-01, OA-201, OA-SYS, OATD-01, OB-003, OB-756, obecabtagene autoleucel, obefazimod, obeticholic acid, obeticholic acid magnesium, obexelimab, obinutuzumab, OBM-POI, OC-701, ocadusertib, ocaratuzumab, OCH-NCNP1, OCR-4715, Octagam 10%, OD-07656, odevixibat, ODS-101, OGB-21501, OGB-21502, olamkicept, olinciguat, olmesartan+dapagliflozin, olodaterol, olodaterol hydrochloride+tiotropium bromide monohydrate 1, olokizumab, olopatadine, OLP-1002, olsalazine, oltipraz, OLX-103, OLX-701, OLX-702A, OM-001, OM-002, OM-85-IN, OM-89, omalizumab, omarigliptin, omeprazole, omeprazole and bicarbonate, omiganan pentahydrochloride, omilancor, OMN-71, ON-104, ONO-4685, OP-2101, opaganib, OPC-163493, opinercept, OpSCF, OPT-101, oral, Oral IL-17 receptor antagonist, orally administered encapsulated lyophilised faecal microbiota, ORBCEL-C, ordesekimab, orelabrutinib, orforglipron, ORI-001, orismilast, ORKA-001, ORKA-002, ORMD-0801, ORMD-0801+ORMD-0901, ORMD-0901, Orthokine, orticumab, Orynotide rhesus theta defensin-1, OsrHSA, OSSM-001, OST-122, otelixizumab, otenaproxesul, otilimab, OTL-104, oxaprozin, OXM-Q3E, Oxy-210, ozagrel hydrochloride, ozanimod, ozenoxacin, ozoralizumab, P-11, PA-9159, PAD4 inhibitor, pafolacianine, PALI-2108, pancreatic beta cell modulators, PanINSULA, pan-KRas inhibitor, Panzyga, PAR2 inhibitor pepducins, paridiprubart, parsaclisib, PB (OA)-5010, PB-718, PBF-677, PBF-680, PBI-100, PBI-4547, PC-114, PDC-APB, PDE4 inhibitor, PDX-02, PDX-06, PEC-Direct, PEC-EnCap, peficitinib, pegapamodutide, pegargiminase, pegbelfermin, PEG-exenatide, peginterferon alfa-2a, peginterferon alfa-2b, PEG-loxenatide, pegozafermin, pegtarazimod, pegylated antianji peptide, PEGylated exenatide, pelubiprofen, pemafibrate, pemafibrate+tofogliflozin, pemirolast, pemvidutide, pentosan polysulfate sodium, Peptide, peptide therapy, peptide-loaded nanoparticles, peresolimab, petrelintide, PF-06480605, PF-06687234, PF-06835375, PF-06835919, PF-06954522, PF-07054894, PF-07261271, PF-07264660, PF-07275315, PF-07832837, PF-07853578, PF-1803, PG-011, PG-101, PG-102, PG-109, PG-201, PGC-fatylated terlipressin, PGDH inhibitors, PGN-001, PGN-OB2, PG-OA-10CN, PH-00011, PH-00012, PHDC-02, phimelanotide, PHIN-214, PHP-1212, PHP-303, PI3K-delta inhibitor, picankibart, piclidenoson, pimecrolimus, pioglitazone, pioglitazone+extended-release metformin, pioglitazone+glimepiride, pioglitazone+metformin, pioglitazone+teneligliptin, PIPE-791, pirfenidone, piroxicam, PIT-565, pitavastatin, PK-101, PK101-002, PKC theta inhibitors, PKX-001, PL-8177, PLA2-activated phospholipid-drug conjugate prodrugs, plexaris, PLM-301, PLN-1474, plonmarlimab, PN-943, PNB-001, PNPLA3 inhibitor, PNPLA3 siRNA therapy, PNV-5032, PNV-6005, POLB-002, polmacoxib, ponesimod, potassium dobesilate, povetacicept, PPAR-gamma agonists, PPS, PPV-06, PR-023, PR-1100, PR-2100, PR-300, PR-600, PRA-023 (tulisokibart), PRA-052, PRA-523, pramlintide+exenatide, pramlintide+human insulin, pranlukast, pranlukast hydrate, PRCL-02, prednisolone, prednisolone sodium phosphate enema, prednisone, preimplantation factor, PrEP-001, PRG-2311, PRIM-DJ2727, PRL-002, PRO-20, ProAgio, Procell, proglumide, Prolastin, proline henggliflozin+retagliptin phosphate+metformin hydrochloride, propionyl-L-carnitine, Prosorba, prostaglandin D2 synthase inhibitors, Protego-PD, protein arginine deiminase IV inhibitor, protein kinase C-theta inhibitors, protein kinase inhibitors, ProTrans, Prozumab, PRP-PBMC autologous cellular therapy, prulifloxacin, prusogliptin, PRV-101, PRV-3279, PS-1, PS-35, PSC-01, psilocybin, psoriasis therapeutics, PSTvl, PT-001, PT-002, PT-101, P-TET, PTG-007, PTG-020, PTG-100, PTPN1 siRNA therapy, PTUPB, PTX-1059, PUL-042, PUR-0110, PUR-1800, PVT-101, PX-04, PX-128, PX-130, PXL-065, PXL-770, PXS-5370, PXS-5382, pyrazole carboxamide analogs, PZ-07/2024, PZH-2109, Q-1804, Q-301, Qaialdo, QB-0208-1, QBECO, QBKPN, QBT-002, QLM-3003, QLR-12018, QN-02, QP-C01, QPCT inhibitor, QRB-001, QRX-008, QUC-398, quisovalimab, QX-002-N, QX-004-N, QX-005-N, QX-006-N, QX-007-N, QX-008-N, QX-009-N, QX-010-N, QX-011-N, QY-101, QY-201, QY-211, QY-301, R-0737072, R-187, R-2487, R-3750, R-491, R-5280, R-552, R-851, rabeximod, rademikibart, radiolabelled FAPI-46, rapcabtagene autoleucel, rapirosiran, rare phytocannabinoids, ravulizumab, RAY-001, RAY-002, RAY-121, RAY-1225, RB-1000, RBM-009, RBM-010, RBN-012759, RBO-0987, RBP4 antagonist, RBX-2660, RC-0165, RC-1416, RCD-405, RCI-003, RCYM-001, RD-1301, rE-4, rebamipide mofetil, recombinant angiotensin converting enzyme 2 (ACE-2) gene therapy/AAV vector, recombinant CTRP12 protein, recombinant human interleukin-1 receptor antagonist, recombinant human interleukin-2, recombinant human lubricin, recombinant human plasma gelsolin, recombinant midismase, redasemtide, reformulated calcipotriol+betamethasone, REGEND-001, regenerative cartilage therapy, REGN-1908-1909, ReJoin, relmacabtagene autoleucel, remestemcel-L, remetinostat, remibrutinib, remogliflozin etabonate, remogliflozin etabonate+teneligliptin, remogliflozin etabonate+vildagliptin, remogliflozin etabonate+vildagliptin+metformin, rencofilstat, renzapride, REP-2139, repaglinide, repaglinide+metformin, repaglinide+metformin hydrochloride, repirinast, repurposed aldesleukin, Repurposed azeliragon, repurposed lenabasum, RES-010, ReS39-I program, resiniferatoxin, reslizumab, resmetirom, resomelagon acetate, RESP-1000 series, RESP-2000 series, RESP-X, retagliptin, retatrutide, retinoic acid, revefenacin, REX-7117, rezpegaldesleukin, RG-125, RG-19, RG-6151, RG-6244, RG-6287, RG-6314, RG-6315, RG-6341, RG-6377, RG-6421, rGDFT1, RgnA-09, RG-NAE005, RGRN-305, RGT-028, RGT-075, RHE-70202, rheumatoid arthritis/poly-articular-course juvenile RA/psoriasis), RHH-646, RhuDex, rifabutin+clarithromycin+clofazimine, rifamycin, rifaquizinone, rifaximin, rilparencel, rilzabrutinib, riociguat, risankizumab, ritlecitinib, rituximab, RJ-4287, RJVA-001, RLS-1496, RLV-102, RMD-1101, RMD-1201, RO-7504109, RO-7507062, rocatinlimab, roconkibart, rofecoxib, roflumilast, ropsacitinib, ROR gamma T inverse agonists, ROR-107, ROR-gamma inverse agonists, rose bengal sodium, rosiglitazone, rosiglitazone+glimepiride, rosiglitazone maleate+metformin hydrochloride, rosiglitazone sodium, rosnilimab, rosuvastatin+telmisartan+amlodipine, RP-005, RP-3128, RP-901, RPH-104, RPI-78, RS-1805, RSBT-001, RSLV-132, RSS-0393, RSVI-301, RT-200, R-TPR-022, RTX-001, RTX-T1D, rupatadine+montelukast, RUTI, ruxolitinib, RXC-008, RYSW-01, Ryzodeg, RZ-629, 5-151128, S1P1 agonist, S1P1 receptor modulator, SAB-01, SAB-03, SAB-142, SAL-0112, SAL-0119, SAL-067, salbutamol, salmeterol, salmeterol xinafoate+fluticasone propionate, salsalate, SAMiRNA program, SAMiRNA-AREG, SAN-903, santamarin derivatives, SAR-441566, SAR-443726, sarecycline, sarilumab, saRNA therapeutics, saroglitazar magnesium, saxagliptin, saxagliptin+dapagliflozin, saxagliptin+dapagliflozin+metformin hydrochloride, saxagliptin+extended-release metformin, saxagliptin+metformin, SB-010, SB-011, SB-012, SB-061, SB-121, SBI-102, SBI-3150, SBP-302, SBT-11-5301, SBT-77-7101, SC-291, SC-451, SCD-044, SCD-153, Sclnow Biotechnology, SCO-094, SCO-116, SCO-267, SCRI-CAR19v3, SCT-5-27, SCT-640A, SCT-650-C, SDC-1801, second generation chitotriosidase 1 inhibitor, second generation lecinoxoids, second generation naltrexone analog, secukinumab, SEFA-1024, SEGRA, seladelpar lysine, selatogrel, selective JAKs inhibitor, selective MCT inhibitor, selective TYK2 inhibitor, seletalisib, seliciclib, SEL-K2, selnoflast, selvigaltin, semaglutide, SER-101, SER-140, SER-287, SER-301, seratrodast, serelaxin, setanaxib, sevelamer hydrochloride, SFA-001, SFA-002, SFA-004, SFX-01, SG-100, SG-2-0776, SG-301, SGM-1019, SGN-102, SGT-510, SGX-201, SH2 domain inhibitor program targeting STAT6, SH-2442, SHC-023, SHC-028, Shiloah-1000, SHO-147, shortened GLP-1, SHR-1654, SHR-1703, SHR-1819, SHR-1905, SHR-2001, SHR-2042, SHR-2173, SHR-3167, SHR-3824, SHR-4597, si-544, SIG-001, SIG-002, SIG-1322, SIG-1451, SIG-1456, SIM-0278, SIM-0708, SIM-335, Sinbaro, SINE compounds, siplizumab, siRNA therapy, SIRT6 gene therapy, SIRT6 inhibitors, Sirtuin6 protein regulators, SIT-022, SIT-033, SIT-047, sitagliptin, sitagliptin+extended-release metformin, sitagliptin+glimepiride+metformin hydrochloride ER, sitagliptin+metformin, sitagliptin+metformin+pioglitazone, sitagliptin+simvastatin, sitagliptin fenilalanil hydrochloride, sitaxentan, sitocabnagene loxiveluecel, sitokiren, SK-01, SK-08, SK-1306X, SK1-I, SKCPT, SKI-O-703, SKL-XYZ, SL-100, SLS-008, SM-06, SM-17, SM-934, small mobile stem cell therapy, SMET-D1, SMUP-IA-01, SN-1011, SN-401, SN-406, SNC-103, SNG-001, SNG-100, SNK-01, SNP-610, SNP-630, sodium chromoglycate, Sodium Hyaluronate, sodium pyruvate, sodium thiosulfate, sofnobrutinib, Sofusa anti-TNF, SOL-116, soluble guanylate cyclase stimulator, sonelokimab, soquelitinib, SOR-101, SOR-102, SOR-103, sotagliflozin, sovleplenib, SP-16, SP5-M001, SP5M-002, spesolimab, SPH-7050, SPH-7854, SPL-5AC, SPL84-23, SPL-891, SPN-0103-009, SPP-004, sprifermin, SPY-001, SPY-002, SPY-003, SPY-004, SPY-120, SPY-130, SPY-230, SQZ-TAC-CeDSR-878, SQZ-TAC-T1D, SR-03, SR-040/041/042, SR-044, SR-047, SR-878, SRI-37330, SRT-015, SSGJ-621, SSS-07, ST-003, ST-1830, ST-266, ST-3870, stapokibart, STAR-0310, STAT3 inhibitor, StemCART, Stemchymal, Stempeucel, STK-009+SYNCAR-001, STM-003, STMC-103H, STMC-106, STNM-01, STP-707, StroMel, STS-01, STSA-1201, suciraslimab, SUDO-286, SUL-238, sulfamethoxazole+trimethoprim, supalutai, SuperMApo, Suplasyn, suplatast tosilate, survodutide, SVP insulin+SVP rapamycin, SVT-201, SWK-002, SY-004, SY-008, SY-009, SYHA-1805, SYHX-1901, SYN-020, SYN-321, synthetic biotic medicine, SYX-5219, SZN-043, T-1123, T1D-ADV210, T-21, T2D-PT201, T-517, tacalcitol, tacrolimus, tadekinig-alfa, TAF-001, tagraxofusp, TAGX-0003, TAK-101, TAKC-02, tamuzimod, tanimilast, TAP-1502, TAP-1503, tapinarof, targeted gene therapy, TAS-5315, Tavo-101, tazarotene, tazarotene+betamethasone dipropionate, tazarotene+clindamycin, TB-001, TB59-2, TB-840, TB-D-004C, TBK1 exosite inhibitor, TBX-301, T-cell expressed protein, TCK-276, TCM-800B, TCR-TREGs, TD-5202, TD-8236, TDI-01, TDM-180935, TDM-Atop01, TDM-Psor01, TDM-Scar01, TDM-SLE01, TE-8105, technetium Tc 99m tilmanocept, technetium[99Tc]methylenediphosphonate, tegoprazan, telazorlimab, telitacicept, TELOMIR-1, temelimab, Tempol, temtokibart, teneligliptin+metformin, teneligliptin hydrobromide, teneligliptin hydrobromide+canagliflozin hydrate, tenoxicam, teplizumab, teprotumumab, TER-101, terbutalin, terguride, TERN-101, TERN-501, tesamorelin, tesnatilimab, testosterone prodrug, testosterone undecanoate, TEV-48574, TEV-53275, TEV-53408, tezepelumab, TFF-HMW-HA, TG-68, TH-104, Thalassophryne nattereri peptide, THB-001, THDBH-101, THDBH-110/THDBH-111, THDBH-120/THDBH-121, theophylline, thioacrylamide compounds, THOR-809, TH-SC01, thyroid hormone receptor beta-selective agonists, TI-520, TI-620, tianagliflozin, tibulizumab, TID-PT101, tildrakizumab, tilpisertib, tilpisertib fosmecarbil, timolumab, tiotropium, tiotropium bromide, tipelukast, tirbanibulin, tirzepatide, TISA-633, tissue-specific immunomodulating bispecific antibodies, tivanisiran, TIX-100, TJ-103, TJC-0316, TJC-0434, TLB-003, TLC-065, TLC-1235, TLC-2716, TLC-3595, TLC-599, TLC-6740, TLL-018, TLR-4 modulator, TLY-004, TLY-012, TMP-269, TMP-501, TNF-2, TNX-1500, TO-210, tocilizumab, tofacitinib, tofacitinib+fingolimod, tofacitinib citrate, tofacitinib citrate extended-release tablet, tofogliflozin, TOL-3021, ToleraCell-001, ToleraCell-002, tolimidone, TollB-001, tonabacase, tonogenchoncel-L, TOP-N44, TOP-N53, TOP-V122, torudokimab, tosufloxacin, tozorakimab, TP-2, TP-317, TP-352, TPX-100, TQ-05510, TQA-3526, TQB-3702, TQC-2731, TQC-2938, TQC-3564, TQC-3721, TQC-3927, TQ-F3083, TQH-2722, TQH-2929, TQH-3821, TQH-3906, TQH-3910, TR-004, TR-8, trabikibart, tralokinumab, tranilast, trans-capsaicin, transcription factor pathway inhibitor, TRE-515, Treg modulator therapy, tregalizumab, trelagliptin succinate, treprostinil, treprostinil diolamine, tretinoin, tretinoin+benzoyl peroxide, TreXTAM, triamcinolone acetonide, Trichomylin, Trichuris suis ova, tricyclic pyrone compounds, trifarotene, TRIV-509, TRN-157, TRPA1 antagonists, TRX-103, TRX-105, TS-0001, TS-20004, TSG-03-117, TSL-0319, TST-801, TT-01, TT-01025, TT-01688, TT-02332, TTAX-03, TTP-273, TTP-RA, TU-5113, tulinercept, tulobuterol, TVB-3567, TXB-0063, TXR-611, TXR-612, TXR-711, TY-705, UA-008, UA-021, UB-221, UBG-T19, UBP-1213, UBT-251, UCB-1381, UCB-9741, ucenprubart, UHE-101, UHE-105, UI-009, UI-010, UI-031, UI-033, UI-034, UI-055, UI-056, UI-068, ularitide, Ulinastatin, ulobetasol, umbilical cord blood-derived mesenchymal stem cell therapy, umbilical cord blood-derived stem cell therapy, umbilical cord-derived mesenchymal stem cells, UMC119-06, UMC119-06-05, umeclidinium bromide, umeclidinium bromide+vilanterol trifenatate, umedaptanib pegol, UN-03, Uni-E4-Fc, UP-421, upadacitinib, upadacitinib+elsubrutinib, ursodeoxycholic acid, ursolic acid, usnoflast, USP-4 inhibitors, ustekinumab, UTAA-09, V-411, VALERGEN-DS, valsartan+celecoxib, valziflocept, vamorolone, Vancocin, vancomycin hydrochloride, vapendavir, VAR 200-03, vardenafil, VB-1953, VB-201, VB-601, VC-005, VCT-220, VCTX-210, VCTX-212, VDAA, VDAD, VDJ-006, VDJ-010, VDJ-142, VDJ-206, VE-202, VE-202-B, VE-5708, VE-5773, vedolizumab, VEGFR targeted DK4/10, vemircopan, venanprubart, VENT-03, verdinexor, verekitug, vesicular monoamine transporter 2 antagonists, vidofludimus, vilamakitug, vilanterol+fluticasone furoate+glycopyrronium bromide, vildagliptin, vildagliptin+metformin, vildagliptin+pioglitazone, vildagliptin+pioglitazone hydrochloride, vildagliptin SR+metformin SR, vipoglanstat hydrogensulfate, Viromed, visepegenatide, VISTA agonist, VITA-100, vixarelimab, VK-0612, VK-2735, VK-2809, VLRX-001, VM-AD, VNA-438, VN-B101, VNLG-152, vobarilizumab, voclosporin, voglibose, voglibose+metformin, volagidemab, volixibat potassium ethanolate hydrate, vonafexor, vonifimod, vonoprazan, vorbipiprant sodium, voriconazole, votucalis, VRN-04, VS-105, VSG-158, VSTM-1 agonist, VT-014, VTH-212, VTP-1000, VTX-2735, VTX-958, VUM-02, vunakizumab, vutiglabridin, VVD-702, VX-150, VX-264, VX-880, VYN-201, VYN-202, W16P-0576, wanpagliflozin, WCDD-301, WD-890, WF-10, WGc-043, Wharton jelly derived mesenchymal stem cells, WM-1R3, WNT inhibitor, WNT pathway agonist antibodies, Wnt pathway modulating antibody, wnt pathway stimulator, WS-012, WXFL-10203614, WXSH-0038, WXSH-0078, WXSH-0150, WXSH-0176, WXSH-0213, XCUR-17, XEN-103, XEN-D0501, xenin-fused peptides, XFB-19, XG-004, XIN-5404, XIN-5494, Xinbang Mesenchymal Stem Cell Injection, XKH-001, XmAb-564, XP-3924, XSTEM-OA, XT-0528, XT-150, XTYW-003, XW-013, XW-014, XW-015, XZ.700, XZP-5610, XZP-5695, XZP-6019, YA-6060, YC-002, YD02-2022, YFQLXB-UC01, YG-1699, YH-001, YH-2000, YH-25487, YH-25724, YH-35324, YH-40863, YHC-1102, YHC-1108, YHC-1131, YIV-906, YJH-0425, YKRH-00020, yogliptin, YR-001, YR-201, Yso2, Yso3, YW-1128, YY-1201, YY-3411, YY-3970, YYC-405, YYH-25487, zabedosertib, zafirlukast, zamaglutenase, zanubrutinib, zasocitinib, ZB-002, ZB-168, ZED-1227, zelasudil, Zemaira, ZeP-3, zetomipzomib, zevaquenabant, ZG-0588, ZGHUMSC-001, ZHB-107-108, zibotentan, ziftomenib, zileuton, zilucoplan, zirconium zr 89 crefmirlimab berdoxam, Zituvio, ZL-1102, ZL-82, ZM-008, ZMC-001, zoledronate disodium, zoledronic acid, ZP-9830, ZPL-521, ZSP-0678, ZSP-1601, ZSYM-008, ZT-001, ZT-002, ZT-003, ZT-01, zuberitamab, zucapsaicin, ZX-2010, ZX-2020, ZX-2021, ZXBT-1158, or ZYBK-2.

### Combination Therapies for Dermatology and Allergic Disease

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), cyclosporine, methotrexate, mycophenolate mofetil, azathioprine, interferon gamma, phosphodiesterase 4 (PDE4) inhibitor such as crisaborole, JAK inhibitor (e.g., ruxolitinib, upadacitinib, abrocitinib, baricitinib), dupilumab, and anti-IL-13 antibody (e.g., tralokinumab).

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as acne. Non-limiting examples of such agents include topical therapies such as benzoyl peroxide, topical retinoids, topical antibiotic, clascoterone, salicylic acid and azelaic acid; and systemic therapies such as doxycycline, minocycline, sarecycline, combined oral contraceptives, spironolactone, and isotretinoin.

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as alopecia areata. Non-limiting examples of such agents include topical therapies such as systemic corticosteroids (such as prednisolone), cyclosporine, azathioprine, methotrexate, sulfasalazine, simvastatin/exetimibe, inosiplex, antihistamines (such as fexofenadine), and oral JAK inhibitors (such as ritlecitinib or brepocitinib).

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as asthma. Non-limiting examples of such agents include inhaled ICS-formoterol (such as budesonide-formoterol), short-acting beta2 agonists (such as albuterol sulfate), leukotriene receptor antagonists (such as montelukast), immunoglobulin E antibodies (such as omalizumab) and long-acting muscarinic antagonists (such as tiotropium).

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as chronic obstructive pulmonary disease (COPD). Non-limiting examples of such agents include short-acting beta2 agonists (such as albuterol sulfate), short-acting muscarinic antagonists (such as aiprtropium), long-acting beta2 agonists (such as olodaterol), and long-acting muscarinic antagonists (such as tiotropium).

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as chronic rhinosinusitis with polyps. Non-limiting examples of such agents include intranasal corticosteroid, or biologics such as benralizumab (targets IL-5), dupilumab (targets IL-13), omalizumab (targets IgE).

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as contact dermatitis. Non-limiting examples of such agents include topical corticosteroids, topical calcineurin inhibitors (such as pimecrolimus, tacrolimus), topical phosphodiesterase 4 inhibitors, such as crisaborole, systemic immunosuppressants and modulators, such as systemic corticosteroids, methotrexate, azathioprine, mycophenolate mofetil, cyclosporine or dupilumab.

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as dermatomyositis. Non-limiting examples of such agents include topical corticosteroids, topical calcineurin inhibitors (such as pimecrolimus, tacrolimus), systemic corticosteroids (such as prednisone), antimalarials (such as hydroxychloroquine, cholorquine, quinacrine), methotrexate, mycophenolate mofetil, intravenous immunoglobulin, rituximab, and JAK inhibitors (such as tofacitinib)

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as esophageal eosinophilia. Non-limiting examples of such agents include systemic corticosteroids (such as budesonide, fluticasone, prednisone), topical corticosteroids, and proton pump inhibitors (such as omeprazole, esomeprazole, pantoprazole and lansoprazole).

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as psoriasis. Non-limiting examples of such agents include topical treatments such as topical corticosteroids (such as betamethasone dipropionate, clobetasol propionate, desoximetasone, diflorasone diacetate, fluocinonide, flurandrenolide, halobetasol propionate, amcinonide, mometasone furoate, triamcinolone acetonide, fluticasone propionate, hydrocortisone valerate, clocortolone pivalate), topical calcineurin inhibitors (such as pimecrolimus, tacrolimus), topical vitamin D analogues (such as calcipotriene, calcitriol, tacalcitol or mazacalcitol), topical retinoids (such as tazarotene); systemic nonbiologic therapies such as methotrexate, phosphodiesterase 4 inhibitors (such as apremilast), immunosuppressants (such as cyclosporine), oral retinoids (such as acitretin), oral Janus kinase inhibitors (such as tofacitinib), fumaric acid esters (such as dimethyl fumarate), systemic immunosuppressants and antimetabolites (such as hydroxyurea, mycophenolate mofetil, azathioprine, leflunomide, tacrolimus and thioguanine); and biologic therapies such as TNF-α inhibitors (such as etanercept, infliximab, adalimumab, certolizumab), IL-12/IL-23 inhibitors (such as ustekinumab), IL-17 inhibitors (such as secukinumab, ixekizumab, brodalumab), and IL-23 inhibitors (such as guselkumab, tildrakizumab, risankizumab).

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as scleroderma. Non-limiting examples of such agents include immunosuppressive treatments (such as methotrexate, mycophenolate mofetil, cyclophosphamide, tocilizumab, and rituximab), and autologous haematopoietic stem cell transplantation.

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a dermatologic condition, such as vitiligo. Non-limiting examples of such agents include topical treatments such as topical corticosteroids, topical calcineurin inhibitors (such as pimecrolimus, tacrolimus), topical vitamin D analogues (such as calcipotriene); and systemic therapies such as oral corticosteroids (such as betamethasone).

Combination Therapies for Inflammatory bowel disease (IBD)

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a gastroenterologic condition such as inflammatory bowel disease (IBD-C, constipation, or IBD-D, diarrhea). Non-limiting examples of such agents for IBD-C include: osmotic laxatives (e.g, PEG), antispasmodics (e.g., hyoscyamine, peppermint oil), secretagogues (linaclotide, lubiprostone, plecanatid, tenapanor), tegaserod, low dose tricyclic antidepressant, serotonin-norephinephrine reuptake inhibitor or brain-gut behaviour therapies. Non-limiting examples of such agents for IBD-D include: Loperamide, bile acid sequestrant (e.g., colestipol), antispasmodics (e.g., hyoscyamine, peppermint oil), rifaximin, low-dose TCA (e.g., amitriptyline, desipramine, eluxadoline), alosetron or serotonin-norephinephrine reuptake inhibitor or brain-gut behaviour therapies.

### Combination Therapies for Ulcerative Colitis

In some embodiments, the anti-IL-10Rαβ VHH1-VHH2 or the anti-IL-10Rαβ VHH1-VHH2-Fc is combined or co-administered with one or more agents useful for the treatment and/or prophylaxis of a gastroenterological condition such as ulcerative colitis (UC) or Crohn's disease (CD). Non-limiting examples of such agents include infliximab, adalimumab, golimumab, vedolizumab, tofacitinib, ustekinumab, natalizumab, mesalamine, diazo-bonded 5-ASA, sulfasalazine, balsalazide, olsalazine, corticosteroids such as budesonide, hydrocortisone, methylprednisolone, and prednisone; immunosuppressants or immunomodulators such as azathioprine and 6-mercaptopurine, cyclosporine, and methotrexate.

### Combination Therapies for Rheumatoid Arthritis (RA)

In some embodiments, the anti-IL-10Rαβ VHH1-VHH2 or the anti-IL-10Rαβ VHH1-VHH2-Fc is combined or co-administered with one or more agents useful for the treatment and/or prophylaxis of rheumatoid arthritis. Non-limiting examples of such agents include disease-modifying antirheumatic drugs (DMARDS), such as hydroxychloroquine, sulfasalazine, methotrexate, and leflunomide; TNF inhibitors (e.g., etanercept, adalimumab, infliximab, golimumab, certolizumab pegol), T cell costimulatory inhibitor, (e.g., abatacept), IL-6 receptor inhibitors (e.g., tocilizumab, sarilumab), anti-CD20 antibody (e.g., rituximab); and JAK inhibitors (e.g., tofacitinib, baricitinib, upadacitinib); NSAIDs, such as ibuprofen, naproxen, and diclofenac; COX-2 inhibitor, such as celecoxib and etoricoxib; steroids and corticosteroids, such as prednisolone and cortisone; and biological agents known for treatment and/or prophylaxis of such conditions, including for example etanercept (e.g., ENBREL), infliximab (e.g., REMICADE), adalimumab (e.g., HUMIRA), anakinra (e.g., KINARET), abatacept (ORENCIA), rituximab (e.g., RITUXAN), certolizumab (e.g., CIMZIA), golimumab (e.g., SIMPONI), and tocilizumab (e.g., ACTEMRA). In some embodiments, a compound of the disclosure is administered with two additional thereapeutic agents useful for the treatment and/or prophylaxis of a rheumatological condition. In some embodiments, agents useful for the treatment and/or prophylaxis of a rheumatological condition include a compound of the disclosure and two additional therapeutic agents, such as methotrexate+leflunomide, methotrexate+sulfasalazine, methotrexate+cyclosporine, methotrexate+hydroxychloroquine and triple therapy treatments hydroxychloroquine+sulfasalazine+methotrexate, hydroxychloroquine+sulfasalazine+leflunomide.

### Systemic Lupus Erythematosus (SLE) or Lupus Nephritis (LN)

In some embodiments, the anti-IL-10Rαβ VHH1-VHH2 or the anti-IL-10Rαβ VHH1-VHH2-Fc is combined or co-administered with one or more agents useful for the treatment and/or prophylaxis of systemic lupus erythematosus (SLE) or lupus nephritis (LN). Non-limiting examples of such agents include immunosuppressive drugs that inhibit activity of the immune system and agents approved for treatment of SLE, such as hydroxychloroquine, steroids and corticosteroids (e.g., prednisone, methylprednisolone), belimumab, azathioprine, methotrexate, cyclophosphamide, mycophenolate and mycophenolate mofetil, cyclosporine, leflunomide, voclosporin, abatacept, anifrolumab, rituximab, NSAIDS, such as naproxen sodium and ibuprofen, antimalarial drugs, such as hydroxychloroquine, calcineurin inhibitors, and tacrolimus.

In some embodiments, the anti-IL-10Rαβ VHH1-VHH2 or the anti-IL-10Rαβ VHH1-VHH2-Fc is combined or co-administered with two or more agents useful for the treatment of LN, such as prednisone+mycophenolic acid analogs, prednisone+mycophenolic acid sodium prednisone+cyclophosphamide, prednisone+tacrolimus, prednisone+voclosporin, prednisone+belimumab+mycophenolic acid analogs, prednisone+belimumab+cyclophosphamide, prednisone+rituximab.

In further embodiments, the anti-IL-10Rαβ VHH1-VHH2 or the anti-IL-10Rαβ VHH1-VHH2-Fc is combined or co-administered with two or more agents useful for the treatment of LN, such as prednisone+mycophenolic acid analogs, prednisone+mycophenolic acid sodium, prednisone+Azathioprine, prednisone+ Tacrolimus, prednisone+cyclosporine, prednisone+mizoribine.

### Combination Therapies for Osteoarthritis (OA)

In some embodiments, the anti-IL-10Rαβ VHH1-VHH2 or the anti-IL-10Rαβ VHH1-VHH2-Fc is combined or co-administered with one or more agents useful for the treatment and/or prophylaxis of osteoarthritis (OA). Non-limiting examples of such agents include nonsteroidal anti-inflammatory drugs (NSAIDs), topical capsaicin, intraarticular glucocorticoid injections, acetaminophen, duloxetine, tramadol, and injectable corticosteroids such as methylprednisolone acetate, triamcinolone acetate, betamethasone acetate and betamethasone sodium phosphate, triamcinolone hexacetonide, and dexamethasone.

### Combination Therapies for Hepatology Diseases

In some embodiments, the anti-IL-10Rα P VHH1-VHH2 or the anti-IL-10Rα P VHH1-VHH2-Fc is combined or co-administered with one or more agents useful for the treatment and/or prophylaxis of a heptatologic condition, such as NAFLD or NASH. Non-limiting examples of such agents include glucagon-like peptide-1 receptor agonists (GLP-1 RA, e.g., liraglutide, semaglutide, tirzepatide), sodium-glucose cotransporter-2 inhibitor (SGLT2i), thiazolidinediones (pioglitazone) and vitamin E.

In some embodiments, the anti-IL-10Rα P VHH1-VHH2 or the anti-IL-10Rα P VHH1-VHH2-Fc is combined or co-administered with one or more agents useful for the treatment and/or prophylaxis of a heptatologic condition, such as primary biliary cholangitis (PBC) or primary sclerosing cholangitis (PSC). Non-limiting examples of such agents include ursodeoxycholic acid or obeticholic acid.

### 12. Kits

Further provided are kits comprising one or more unitary doses of one or more of the anti-IL-10Rα VHH, the anti-IL-10Rβ VHH, the anti-IL-10Rαβ VHH1-VHH2, and the anti-IL-10RaP VHH1-VHH2-Fc, described herein, including polynucleotides encoding, and pharmaceutical compositions and conjugates thereof.

In some embodiments, the kits comprise one or more containers (e.g., vials, ampules) comprising one or more of one or more of the anti-IL-10Rα VHH, the anti-IL-10Rβ VHH, the anti-IL-10Rαβ VHH1-VHH2, and the anti-IL-10Rαβ VHH1-VHH2-Fc, described herein, polynucleotides encoding such anti-IL-10Rα VHH, anti-IL-10Rβ VHH, anti-IL-10Rα VHH1-VHH2, and anti-IL-10Rαβ VHH1-VHH2-Fc, LNPs or pharmaceutical compositions containing such anti-IL-10Rα VHH, anti-IL-10Rβ VHH, anti-IL-10Rαβ VHH1-VHH2, and anti-IL-10Rαβ VHH1-VHH2-Fc. Further provided are kits comprising one or more unitary doses of the anti-IL-10Rα VHH, the anti-IL-10Rβ VHH, the anti-IL-10Rαβ VHH1-VHH2, and the anti-IL-10Rαβ VHH1-VHH2-Fc, as described herein, in one or more delivery devices, e.g., pre-loaded syringes.

In one embodiment, kits comprise one or more unitary doses of the anti-IL-10Rα VHH, the anti-IL-10Rβ VHH, the anti-IL-10Rαβ VHH1-VHH2, and the anti-IL-10Rαβ VHH1-VHH2-Fc, as described herein, in combination with one or more (e.g., one, two, three, one or two, or one to three) additional therapeutic agents. In some embodiments, the kits comprise one or more unitary doses of the one or more unitary doses of the anti-IL-10Rα VHH, the anti-IL-10Rβ VHH, the anti-IL-10Rαβ VHH1-VHH2, and the anti-IL-10Rαβ VHH1-VHH2-Fc, as described herein, or the polynucleotide or polynucleotides encoding, in one or more containers. In some embodiments, the kits comprise one or more unitary doses of the one or more unitary doses of the anti-IL-10Rα VHH, the anti-IL-10Rβ VHH, the anti-IL-10Rαβ VHH1-VHH2, and the anti-IL-10Rαβ VHH1-VHH2-Fc, as described herein, and a second agent for treating an inflammatory disease in separate containers.

### EXAMPLES

The following examples are offered to illustrate, but not to limit the claimed invention.

### Example 1

### Anti-IL-10Rα and Anti-IL-10Rβ Single Domain Antigen Binder Discovery

In this example, we describe discovery of single domain antigen binders (VHH) that specifically bind to human interleukin-10 receptor alpha subunit (IL-10Rα; IL-10RA; CD210; NCBI Gene ID 3587) or human interleukin-10 receptor beta subunit hIL-10Rβ; IL-10RB; CDW210B; NCBI Gene ID 3588). Two alpacas were immunized with hIL-10Rα extracellular domain, two alpacas were immunized with hIL-10Rβ extracellular domain, and two alpacas were immunized with a hIL-10Rα-IgG1/hIL-10Rβ-IgG1 heterodimer hFc fusion protein. Alpaca immunization was followed by immune phage library generation and biopanning.

### Methods

To generate single domain antigen binders (VHH) that specifically bind to human IL-10Rα or human IL-10Rβ, for each immunization group, two alpacas were immunized with the extracellular domain (ECD) of hIL-10Rα (Uniprot:Q13651; SEQ ID NO: 719), the extracellular domain of hIL-10Rβ (Uniprot:Q08334; SEQ ID NO: 720), or a hIL-10Rα-IgG1/hIL-10Rβ-IgG1 heterodimer hFc fusion (SEQ ID NOs: 721 and 722).

hIL-10Rα ECD (UniProtKB Q13651, His22-ASN235)
(SEQ ID NO: 719)
HGTELPSPPSVWFEAEFFHHILHWTPIPNQSESTCYEVALLRYGIESWNSISNCSQTLSYDLTA
VTLDLYHSNGYRARVRAVDGSRHSNWTVTNTRFSVDEVTLTVGSVNLEIHNGFILGKIQLPRPK
MAPANDTYESIFSHFREYEIAIRKVPGNFTFTHKKVKHENFSLLTSGEVGEFCVQVKPSVASRS
NKGMWSKEECISLTRQYFTVIN
hIL-10Rβ ECD (UniProtKB Q08334, Met20-Ser220)
(SEQ ID NO: 720)
MVPPPENVRMNSVNFKNILQWESPAFAKGNLTFTAQYLSYRIFQDKCMNTTLTECDFSSLSKYG
DHTLRVRAEFADEHSDWVNITFCPVDDTIIGPPGMQVEVLADSLHMRFLAPKIENEYETWTMKN
VYNSWTYNVQYWKNGTDEKFQITPQYDFEVLRNLEPWTTYCVQVRGFLPDRNKAGEWSEPVCEQ
TTHDETVPS
hIL-10Rα hG1-Fc SAV + R/hIL-10Rβ Fc W heterodimer
Chain 1
(SEQ ID NO: 721)
HGTELPSPPSVWFEAEFFHHILHWTPIPNQSESTCYEVALLRYGIESWNSISNCSQTLSYDLTA
VTLDLYHSNGYRARVRAVDGSRHSNWTVTNTRFSVDEVTLTVGSVNLEIHNGFILGKIQLPRPK
MAPANDTYESIFSHFREYEIAIRKVPGNFTFTHKKVKHENFSLLTSGEVGEFCVQVKPSVASRS
NKGMWSKEECISLTRQYFTVINDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNK
ALPAPIEKTISKAKGQPREPQVYTLPPSREEMTKNQVSLSCAVKGFYPSDIAVEWESNGQPENN
YKTTPPVLDSDGSFFLVSKLTVDKSRWQQGNVFSCSVMHEALHNRYTQKSLSLSPGK
Chain 2
(SEQ ID NO: 722)
MVPPPENVRMNSVNFKNILQWESPAFAKGNLTFTAQYLSYRIFQDKCMNTTLTECDFSSLSKYG
DHTLRVRAEFADEHSDWVNITFCPVDDTIIGPPGMQVEVLADSLHMRFLAPKIENEYETWTMKN
VYNSWTYNVQYWKNGTDEKFQITPQYDFEVLRNLEPWTTYCVQVRGFLPDRNKAGEWSEPVCEQ
TTHDETVPSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKA
KGQPREPQVYTLPPSREEMTKNQVSLWCLVKGFYPSDIAVEWESNGQPENNYKTTPPVLDSDGS
FFLYSKLTVDKSRWQQGNVFSCSVMHEALHNHYTQKSLSLSPGK

Two alpacas were immunized per immunogen by subcutaneous injection at a dose of 1 mg for five total times at two-week intervals at Lampire Biological Laboratories, Pipersville, PA. Serum was collected and the titer was evaluated by IL-10Rα and IL-10Rβ ELISA on days 35 and 49. Peripheral blood lymphocytes were isolated from a 50 ml blood sample on day 49 and day 63 via Lymphoprep Density Gradient Medium (Stemcell Technologies, Cat. #07581) and SepMate peripheral blood mononuclear cell (PBMC) Isolation Tubes (Stemcell Technologies, Cat. #85450), yielding about 10⁸ B cells. RNA was extracted from B cells via Qiagen RNeasy Mini Kit (Qiagen, Cat. #74104) and converted into cDNA to construct the nanobody phage library. The VHH regions were obtained via two-step PCR, with fragment size of about 400 bp. The VHH product and the vector, pMES4 phagemid (Creative biogene) were digested with NcoI-Hf and Eco91I-Hf and subsequently ligated. After ligation, the products were transformed into Escherichia coli TG1 cells by electroporation. Then, the transformants were enriched in growth medium and planted on plates. Finally, the library size was estimated by counting the number of colonies. The resulting TG1 library stock was then infected with M13K07 helper phage (Antibody Design lab, Cat. #PHOIOL) to obtain a library of VHH-presenting phages. Rescue with helper phage M13K07 and polyethylene glycol precipitation was performed as described previously (Phage Display: Methods and Protocols, Methods in Molecular Biology, 1701, Michael Hust and Theam Soon Lim, editors, 1st edition, 2018, Humana, ISBN-13: 978-1493974467). Specifically, E. coli TG1 cells grown in 2×YT medium supplemented with 50 μg/mL ampicillin and 2% glucose at 37° C. were inoculated in log-phase with M13K07 helper phages and incubated at 37° C. for 30 minutes without shaking. Afterwards, the bacterial culture was centrifuged at 2800×g for 10 minutes, the medium was replaced by 2×YT containing 50 μg/mL ampicillin and 25 μg/mL kanamycin, and the culture was incubated overnight while shaking at 37° C. Subsequently, the material was centrifuged and the supernatant was polyethylene glycol (PEG) precipitated (20% PEG 6000 in 2.5 M NaCl) at 4° C. for 1 hour. Phages were spun down, and the pellet was resuspended in 1 mL PBS.

The VHH specifically binding to hIL-10Rα or hIL-10Rβ were selected by phage display. Phage library was incubated with hIL-10Rα or hIL-10Rβ that were directly immobilized on NUNC™ MaxiSorp™ 96-well plates (Thermo Scientific, cat #439454). Non-specific VHH-phage was washed away with PBS-0.05% Tween 20 and bound phage was eluted with 0.1 M Glycine-HCl, pH 2.3 and neutralized by Tris-HCL, pH 10.2. The eluted phage was then used to infect TG1 cells for subsequent rounds of selection to enrich for anti-hIL-10Rα or anti-hIL-10Rβ specific VHHs. VHH-phage enrichment was monitored by comparing the eluted phage titers from wells that contained hIL-10Rα or hIL-10Rβ to that of wells which contained no proteins in each round of selection. Additionally, the stringency of the biopanning was increased in each round of selection to ensure specific binding by increasing the number of washes and alternating blocking reagents from bovine serum albumin (BSA) to non-fat milk. Phage from enriched rounds of selection were then plated for single colonies to be screened.

### Results

Phage from enriched rounds of selection were then plated for single colonies to be screened by phage ELISA. About 4000 colonies were screened by phage ELISA and 220 positive hits with unique sequences. Phagemid of ELISA positive clones were transduced into E. coli strain SS320 for soluble VHH production in the periplasm of the bacteria. The expression of the VHH was induced using MagicMedia™ E. coli Expression Medium (Invitrogen, Cat #K6803) at 30° C. for 22 hours. Soluble VHH was extracted by the osmotic shock method after 1 freeze-thaw cycle using the extraction buffer, containing Tris-HCl, pH 8.0, 1 mM EDTA, 20% sucrose in the presence of cOmplete Protease Inhibitor Cocktail tablets (Sigma, Cat #11697498001). The supernatant of the periplasmic extract (PPE) containing soluble VHH was extracted and screened binding to hIL-10Rα ECD, hIL-10Rβ ECD and hIL-10Rα hG1-Fc_SAV+R/hIL-10Rβ Fc_W heterodimer by ELISA. In addition, we tested PPE binding to HEK blue IL-10 reporter cell (Invivogen, cat #hkb-IL-10) by FACS. In summary, we identified 23 VHH that specifically bound hIL-10Rα (ELISA binding signal >0.2 to hIL-10Rα ECD and IL-10Rα/hIL-10Rβ heterodimer, FACS MFI Ratio>2.0 to HEK blue parental cell) and
12 VHH that specifically bound hIL-10Rβ (ELISA binding signal >0.2 to hIL-10Rβ ECD and IL-10Rα/hIL-10Rβ heterodimer, FACS MFI Ratio>2.0 to HEK blue parental cell). PPE ELISA and FACS screening data are summarized in Tables 1 and 2. The clone summary for anti-IL-10Rα VHH is provided in Table 1 and the amino acid sequences are provided in Table B. The clone summary for anti-IL-10Rβ VHH is provided in Table 2 and the amino acid sequences are provided in Table D.

TABLE 1
Anti-IL-10Rα VHH

| | | | PPE ELISA | | |
|---|---|---|---|---|---|
| | | | IL-10Rα/IL- | PPE | |
| | PPE ELISA | PPE ELISA | 10Rβ | FACS | SEQ |
| Clone | hIL-10Rα | hIL-10Rβ | Heterodimer | MFI | ID |
| name | (OD 450 nm) | (OD 450 nm) | (OD 450 nm) | Ratio | NO: |
| A01 | 1.338 | 0.053 | 1.071 | 2.0 | 210 |
| A06 | 1.066 | 0.135 | 1.112 | 3.3 | 214 |
| A09 | 0.310 | 0.053 | 0.758 | 8.6 | 216 |
| B07 | 1.058 | 0.052 | 1.233 | 2.0 | 218 |
| B08 | 0.628 | 0.054 | 0.820 | 2.4 | 221 |
| C09 | 0.768 | 0.056 | 0.874 | 4.4 | 225 |
| D08 | 0.996 | 0.057 | 1.012 | 5.6 | 228 |
| D14 | 0.274 | 0.054 | 0.525 | 2.0 | 231 |
| E07 | 0.215 | 0.052 | 0.699 | 5.0 | 236 |
| E15 | 0.94 | 0.054 | 0.926 | 5.7 | 242 |
| F09 | 0.222 | 0.054 | 0.525 | 2.0 | 245 |
| F10 | 0.332 | 0.056 | 0.576 | 2.0 | 247 |
| F11 | 0.835 | 0.053 | 0.847 | 2.8 | 250 |
| F21 | 0.900 | 0.094 | 0.974 | 9.7 | 254 |
| G22 | 0.294 | 0.060 | 0.715 | 8.2 | 257 |
| G23 | 0.896 | 0.160 | 0.872 | 3.6 | 261 |
| H22 | 0.503 | 0.079 | 0.734 | 2.6 | 265 |
| I04 | 0.267 | 0.067 | 0.555 | 3.3 | 268 |
| J13 | 0.701 | 0.056 | 0.790 | 10.0 | 271 |
| J24 | 0.782 | 0.056 | 0.904 | 2.6 | 275 |
| L05 | 0.430 | 0.057 | 0.862 | 2.8 | 279 |
| L11 | 0.235 | 0.060 | 0.836 | 4.6 | 282 |
| N16 | 0.273 | 0.058 | 0.537 | 4.6 | 286 |

TABLE 2
Anti-IL-10Rβ VHH

| | | | PPE ELISA | | |
|---|---|---|---|---|---|
| | | | IL-10Rα/IL- | PPE | |
| | PPE ELISA | PPE ELISA | 10Rβ | FACS | SEQ |
| Clone | hIL-10Rα | hIL-10Rβ | Heterodimer | MFI | ID |
| name | (OD 450 nm) | (OD 450 nm) | (OD 450 nm) | Ratio | NO: |
| A02 | 0.070 | 1.168 | 0.306 | 2.0 | 446 |
| A05 | 0.058 | 2.193 | 2.501 | 2.0 | 450 |
| B10 | 0.074 | 2.670 | 1.406 | 14.7 | 453 |
| B12 | 0.073 | 2.160 | 0.302 | 4.6 | 456 |
| C01 | 0.084 | 2.680 | 0.533 | 3.6 | 458 |
| C04 | 0.074 | 2.437 | 0.992 | 18.0 | 461 |
| C10 | 0.069 | 1.470 | 0.233 | 9.8 | 463 |
| D01 | 0.070 | 1.292 | 0.217 | 7.2 | 465 |
| E08 | 0.073 | 2.704 | 2.672 | 5.0 | 467 |
| E10 | 0.070 | 2.107 | 0.499 | 4.7 | 469 |
| G01 | 0.088 | 2.830 | 1.297 | 8.2 | 471 |
| G02 | 0.100 | 0.117 | 0.679 | 17.7 | 473 |

### Example 2

### VHH Format Exploration and Characterization

In this example, we describe tandem anti-IL-10Rα VHH and anti-IL-10Rβ VHH format exploration with linker length variation between the anti-IL-10Rα and anti-IL-10Rβ VHHs and compare monovalent versus bivalent format using a HEK293T IL-10R-pSTAT3 luciferase reporter assay.

### Methods

Tandem VHH1-VHH2 construct generation. To explore the VHH format and determine the potency impact of linker length between tandem VHHs, we generated DNA constructs of IL-10Rβ VHH (A05) and IL-10Rα VHH (A01) and separated by a panel of Gly-Ser linker length variants (e.g. GS (SEQ ID NO: 713), GGGS (SEQ ID NO: 715), GGSGGS (SEQ ID NO: 716), GGGSGGGS (SEQ ID NO: 717), GGSGGSGGS (SEQ ID NO: 718)) with HIS6 tag as VHH1-VHH2-His format or with Gly-Ser (“GS”) linker followed by CH2-CH3 (DKTHTCPPCPAPEAAGAPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQV YTLPPSREEMTKNQVSLTCLVKGFYPSDIAVEWESNGQPENNYKTTPPVLDSDGSFFLYSKLTV DKSRWQQGNVFSCSVMHEALHNHYTQKSLSLSPGK (SEQ ID NO: 723)) at3′ end as tandem VHH1-VHH2-Fc format (VHH1-GS linker-VHH2-GS-CH2-CH3). DNA inserts codon biased for expression in human cells were cloned into pcDNA3.1 vector (Genscript) for small scale expression in Expi293 cells (ThermoFisher, Cat #A14527). Expi293 cells transfection was conducted according to manufacturer's protocol. One microgram total DNA was used per ml of transfection. DNA was mixed with diluted Expifectamine reagent and incubated at room temperature for 20 minutes, the DNA transfection complex was added to Expi293 cells at 3 million per ml. Five days post-transfection, the clarified supernatant was harvested and purified by Mag Sepharose PrismA beads (Cytiva, Cat #17550001) or Ni Mag Sepharose (Cytiva, Cat #28967390). Purified VHHs were profiled in HEK293T IL-10R-pSTAT3 reporter assay.

HEK293T IL-10R-pSTAT3 reporter assay. IL-10R pSTAT3 luciferase reporter cell line was developed by overexpressing the human IL-10Rα, human IL-10Rβ, and a Firefly luciferase gene driven by STAT3 response elements in HEK293T cells. One day before the assay, culture medium was used to prepare sample plate with eight points dose-response (1:5 serial dilution). Recombinant human IL-10 (50 ng/mL PeproTech Cat #200-10) was used as positive control. Reporter cells were detached from plate using TrypLE™ (Thermo Fisher Scientific) and washed with cell culture media. Cell concentration was adjusted to 8×10⁴ cells/ml and 25 μl (2K cells) of cell suspension was added into each well of solid white tissue culture (TC) 384-well plate (Greiner Cat #781080). On the assay day, five microliters of VHH sample were added to the cell plate and incubated at 37° C. for 3 hours. Thirty microliters of Bio-Glo™ Reagent (Promega Cat #G7941) was added into each assay well followed by a brief spin at 1000 rpm. Plate was then incubated at room temperature for 10 mins and read by EnVision plate reader (Revvity).

The luminescence readout for VHHs treated wells was normalized to average signal from positive (IL-10) and negative (media) controls and expressed as % IL-10R agonist:

$$\% IL - 10 R agonist = \frac{( ( Luminescence sample - Luminescence 0 \% activity )}{( Luminescence 100 \% activity - Luminescence 0 \% activity ) ) * 100}$$

$$Where$$

$$0 \% IL - 10 R agonist = Signal in wells with media control$$

$$100 \% IL - 10 R agonist = Signal in wells with IL - 10 ( 50 ng / mL )$$

EC₅₀ values were calculated based on log concentration of drug and percent of IL-10 signal using a 4-parameter variable slope nonlinear regression model and least square curve fit (Gilead Sciences, Foster City, CA).

$$y = \frac{100 \times E {C}_{50}^{m}}{E {C}_{50}^{m} + {[ A ]}^{m}}$$

where y=% Activity, m=Hill coefficient, and [A]=Agonist concentration.
EC₅₀ values are derived from the average of duplicate data points for each antibody concentration.
Eₘₐₓ is defined as the maximum normalized percentage of IL-10R agonist achieved in the assay.

### Results

The EC₅₀ and Eₘₐₓ values are summarized in Table 3. This data is consistent with the conclusion that the linker length has impact on EC₅₀ but not the maximum agonist activity (Eₘₐₓ) of these VHHs. The amino acid sequence of anti-IL-10Rα VHH A01 is provided in Table B and the amino acid sequence of anti-IL-10Rβ VHH A05 is provided in Table D.

TABLE 3
Linker Length Affects the Potency of αIL-10Rαβ
Agonist Tandem VHH-linker-VHHs

| VHH-VHH | | | |
|---|---|---|---|
| No. | Construct | EC₅₀(nM) | Eₘₐₓ(%) |
| 603 | A05-GGGS-A01_His | 3.8 | 22 |
| 115 | A05_GGSGGS_A01_His | 0.50 | 18 |
| 112 | A05_GGSGGSGGS_A01_His | 0.35 | 14 |
| 111 | A05_GGGSGGGS_A01_His | 0.23 | 17 |
| 110 | A05_GS_A01_His | 0.44 | 20 |

IL-10 forms a dimer and engages two IL-10Rα and two IL-10Rβ to initiate a downstream signal. To determine the avidity of VHHs required for IL-10R agonist activity, bivalent VHH-VHH-Fc format and monovalent VHH-VHH-His format from anti-IL-10Rβ VHH A05 and anti-IL-10Rα VHH A01 were generated and profiled in a HEK293T IL-10R-pSTAT3 reporter assay. The EC₅₀ and Eₘₐₓ are summarized in Table 4. This data is consistent with the conclusion that valency has significant impact on EC₅₀ and maximum agonist activity (Eₘₐₓ) of the A05-A01 tandem VHHs.

TABLE 4
Valency Affects the Potency of αIL-10Rαβ
Agonist Tandem VHH-linker-VHHs

| VHH- | | | | |
|---|---|---|---|---|
| VHH | | | EC₅₀ | Emax |
| No. | Construct | Valency | (nM) | (%) |
| 117 | A05_GGSGGSGGS_A01_hG4 Fc | 2 | 0.071 | 36 |
| 116 | A05_GGGSGGGS_A01_hG4 Fc | 2 | 0.047 | 39 |
| 112 | A05_GGSGGSGGS_A01_His | 1 | 0.351 | 14 |
| 111 | A05_GGGSGGGS_A01_His | 1 | 0.225 | 17 |

### Example 3

### IL-10R Agonistic Potency of Tandem VHH1-VHH2 Bispecific Molecules in a HEK293T IL-10R-pSTAT3 Luciferase Reporter Assay

In this example, we evaluated the IL-10R agonistic potency of αIL-10Rα-IL-10Rβ tandem VHH1-VHH2 bispecific molecules, measured as EC₅₀ and Eₘₐₓ in a HEK293T IL-10R-pSTAT3 luciferase reporter assay.

### Methods

A panel of αIL-10Rα-IL-10Rβ tandem VHH1-VHH2 bispecific molecule combinations from 23 IL-10Rα VHH sequences and 12 IL-10Rβ VHH sequences in both orientations using VHH1-VHH2-His (monovalent) or VHH1-VHH2-Fc (bivalent) formats were evaluated. DNA inserts codon biased for expression in human host cells were cloned into pcDNA3.1 vector (Genscript) for small scale expression in Expi293 cells (ThermoFisher, Cat #A14527) and purified by Mag Sepharose PrismA beads (Cytiva, Cat #17550001) and Ni Mag Sepharose (Cytiva, Cat #28967390), as described above.

### Results

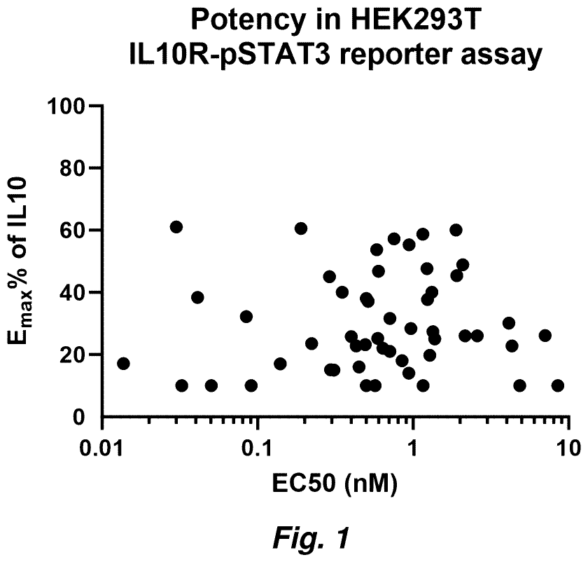

A total of 873 αIL-10Rα-IL-10Rβ tandem VHH1-VHH2 bispecific molecules were evaluated for IL-10R agonistic potency in a HEK293T IL 10R-pSTAT3 luciferase reporter assay to determine agonistic activities as described above. Recombinant human IL-10 induced a robust receptor activation (rIL-10) with 420 pM EC₅₀, whereas 51 tandem VHH1-VHH2 bispecific molecules achieved an Emax value higher than 10% of IL-10 with EC50 ranging from 30 pM to 8.5 nM. The results are depicted in FIG. 1 and summarized in Table 5. αIL-10Rα-IL-10Rβ tandem VHH1-VHH2 bispecific molecules having C10 αIL-10Rα antigen binding domain induced higher T cell IFN-γ secretion and were not pursued further. Two alpaca αIL-10Rα-IL-10Rβ tandem VHH-VHH molecules—877 and 284—demonstrated higher agonistic activities in comparison to other tested αIL-10Rα-IL 10Rβ tandem VHH1-VHH2 molecules. The amino acid sequences of αIL-10Rα VHH are provided in Table B. The amino acid sequences of αIL-10Rβ VHH are provided in Table D. The amino acid sequences of αIL-10Rα-IL-10Rβ tandem VHH1-VHH2, including 877 and 284, are provided in Table E. The amino acid sequences of αIL-10Rα-IL-10Rβ tandem VHH1-VHH2-Fc, including 877 and 284, are provided in Table F.

TABLE 5
Agonist Activities of Anti-IL-10Rαβ VHH1-VHH2
in HEK293T IL-10R-pSTAT3 Luciferase Reporter Assay

| Anti-IL-10Rαβ | | |
|---|---|---|
| VHH1-VHH2 | EC₅₀ PM | Eₘₐₓ % of rIL-10 |
| E07-A02 | 30 | 61 |
| E07-A05 | 190 | 60.5 |
| C10-F09 | 1883.9 | 60 |
| C10-F10 | 1156.8 | 58.7 |
| C10-104 | 755.1 | 57.2 |
| C10-N16 | 942.6 | 55.3 |
| C10-E07 | 582.5 | 53.7 |
| C10-B08 | 2088.9 | 48.9 |
| C10-J13 | 1228.7 | 47.6 |
| I04-A05 | 600 | 46.8 |
| C10-A06 | 1909.2 | 45.4 |
| D08-A05 | 290 | 45 |
| A06-A05 | 1320 | 40 |
| I04-A02 | 350 | 40 |
| F11-C04 | 41.1 | 38.4 |
| A06-A02 | 500 | 38 |
| C10-E15 | 1239.4 | 37.7 |
| B10-A01 | 514 | 37.1 |
| G22-B12 | 84.5 | 32.2 |
| E15-A05 | 710 | 31.6 |
| C10-F21 | 4126 | 30.1 |
| C10-C09 | 969.2 | 28.4 |
| F11-B12 | 1342.5 | 27.4 |
| L05-B12 | 7052.4 | 26.1 |
| C09-A02 | 2590 | 26 |
| G23-B12 | 2157.4 | 26 |
| N16-A05 | 400 | 25.8 |
| C10-D08 | 591.5 | 25.2 |
| H22-B12 | 1376.8 | 25 |
| L11-B12 | 222.5 | 23.5 |
| C10-G22 | 493.4 | 23.2 |
| F09-A05 | 430 | 22.8 |
| G23-D01 | 4327.6 | 22.8 |
| D08-A02 | 640 | 22 |
| J13-A02 | 710 | 21 |
| J13-A05 | 1280 | 19.8 |
| F21-A02 | 850 | 18 |
| G22-C04 | 13.7 | 17.1 |
| L11-B10 | 140 | 17 |
| N16-A02 | 450 | 16 |
| D14-B12 | 294.5 | 15.1 |
| E15-A02 | 310 | 15 |
| F09-A02 | 940 | 14 |
| A09-D01 | 8511.5 | 10 |
| B08-A05 | 4860 | 10 |
| E07-C04 | 500.2 | 10 |
| I04-C04 | 50.5 | 10 |
| C09-C04 | 90.9 | 10 |
| A02-I04 | 1160 | 10 |
| F10-A02 | 570 | 10 |
| F09-C04 | 32.6 | 10 |

### Example 4

### VHH Humanization, Affinity Modulation and Sequence Liability Mitigation

In this example, we describe the engineering process of humanization, affinity modulation and sequence liability mitigation in four binding domains: IL-10Rα VHH (E07, SEQ ID NO: 236) and IL-10Rβ VHH (A05, SEQ ID NO:450); and IL-10Rα VHH (F11, SEQ ID NO: 250) and IL-10Rβ VHH (C04, SEQ ID NO:461). The amino acid sequences of alpaca VHH E07 and F11, and consensus and humanized sequences thereof, are provided in Table B. The amino acid sequences of alpaca VHH A05 and C04, and consensus and humanized sequences thereof, are provided in Table D.

### Methods

Protein expression and purification methods: Designed molecules were codon optimized for CHO cell expression, synthesized, and cloned as bivalent VHH-hIgG1 LALA P331S or VHH-IgG1 LALA G237A or VHH-hIgG1 WT Fc constructs, including a short 1×Gly-Ser between the VHH and the Fc. Plasmids were transiently transfected at 1 ml and/or 40 ml scale using Expi293 (Cat: A14635, Thermofisher). Tandem VHH-Fc constructs were purified by Protein A affinity chromatography (5 mL HiTrap MabSelect-SuRe column from Cytiva, Catalogue Number: 11003495) and size exclusion chromatography (HiLoad 16/600 Superdex 200 pg column from Cytiva, Catalogue Number: 28989335). AKTA Pure 25 chromatography system (Cytiva, Catalogue Number: 29018226) was used to carry out the purification. Sample purity and quality were assessed by analytical size exclusion chromatography (SEC), SDS-PAGE, and mass spectrometry.

SPR by Carterra LSA: Molecules were tested for binding to IL-10Rα or IL-10Rβ. Binding affinity kinetic experiments were performed on a Carterra LSA instrument equipped with PAGHC30M chip type (Carterra-bio). 0.5 μg/ml VHH-hFc in running buffer 1×HBSTE (HEPES-buffered saline, 0.05% Tween 20 and 3 mM EDTA) was captured for 3 minutes with a five-fold dilution series of IL-10Rα or IL-10Rβ, starting at 500 nM in 1×HBSTE, then sequentially injected onto the chip surface. For each concentration, the antigen was injected for 5 minutes (association phase), followed by running buffer injection for 10 minutes (dissociation phase). Two regeneration cycles of 15 seconds were performed between each dilution series by injecting 10 mM Glycine pH 1.5 on the chip surface. The data were analyzed using the Carterra Kinetics analysis software using a 1:1 Langmuir binding model to determine apparent association (ka) and dissociation (kd) kinetic rate constants and binding affinity constants (K_{D}).

Molecular assessment: All samples were buffer exchanged into 20 mM Histidine, pH 5.8 and diluted to 1 mg/mL prior for Molecular Assessment analysis.

Differential Scanning Fluorimetry (DSF): Intrinsic fluorescence of samples (1 mg/mL in 20 mM Histidine, pH 5.8) were measured in duplicates in the Uni tubes (Unchained Labs, 201-1009) using the UNcle instrument (UNchained Labs) with a thermal ramp of 1° C./min from 25° C. to 95° C. Tonset was defined as the onset of protein unfolding and melting temperature™ was defined as the maximum value of the first derivative of the BCM trace using UNcle data analysis software (version 6.01) (unchainedlabs.com/uncle/). The aggregation temperature (Tagg) was calculated using the intensity of scattered light at 266 or 473 nm.

50C1W stress: 500 μL of samples (1 mg/mL in 20 mM Histidine, pH 5.8) were transferred into a sterile screw-capped tubes and incubated in a 50° C. incubator (Thermo Scientific, Model #51028066) for 7 days. Stressed samples were analyzed along with the TO unstressed controls.

aSEC: Analytical size exclusion chromatography (aSEC) is performed on a Vanquish UHPLC duo pump system (Thermo Fisher) with an Acquity UPLC Protein BEH SEC column (Waters, 200A, 1.7 μm, 4.6×150 mm, P/N186005225). The running buffer was 200 mM KH₂PO₄, 250 mM KCl, pH 6.0. The method was performed at a flow rate of 0.4 mL/min for 7 min. Two (2) μg sample was injected to the column, and signals at 280 nm were collected for peak analysis and integration.

### Results

### Anti-IL-10Rα E07 VHH Engineering

IL-10Rα VHH (E07) Humanization. The humanness of alpaca E07 VHH (SEQ ID NO: 236) was assessed using BioPhi (Prihoda, et al., MAbs. (2022) 14(1):2020203; biophi.dichlab.org). The alpaca E07 VHH was found to contain 69% sequence similarity to human framework IGHV3-23 and human IGHV3-23 was chosen as the acceptor framework for humanization. Homology models of alpaca E07 VHH and the human IGHV3-23*01 germline sequence were generated using ImmuneBuilder (Abanades, et al., Commun Biol (2023) 6(1):575; github.com/brennanaba/ImmuneBuilder) and viewed in PyMol (pymol.org). All residues within 5 Å of complementarity determining regions (CDRs), defined by Kabat, were defined as “CDR-supporting” residues and were considered for grafting from the alpaca framework, in addition to Kabat CDRs, onto the human acceptor framework. All residues that differ between alpaca and human that were greater than 5 Å away from Kabat CDRs were left human. Only positions 2, 27, 37, 44, 45, 47, 49, 71, and 94 (according to Kabat numbering) of the CDR-supporting residues differ between the alpaca E07 VHH and the human IGHV3-23 framework. Further, alpaca E07 VHH contains a two amino acid alanine+serine (AS) N-terminal extension. However, the N-terminal AS extension is not native in the encoded human IGHV3-23*01 germline and was removed in all humanized variants. A humanized huE07 VHH (993; SEQ ID NO: 724) was designed with all alpaca Kabat CDRs plus all CDR-supporting residues grafted onto the human framework as the starting point for further humanization.

(SEQ ID NO: 724)
ELQLLESGGGLVQPGGSLRLSCAASGRTFSAYGMGWFRQAPGKER
EFVAAIRWIGDSTYYADSVKGRFTISKDNSKNTLYLQMNSLRAED
TAVYYCAAKTSVTSYLSEKSYNYWGQGTMVTVSS

To increase the percent humanness of huE07 VHH, single and/or double amino acid substitutions from alpaca back to human were made in the CDR-supporting residues of humanized VHHs. Surface plasmon resonance (SPR by Carterra) binding experiments were carried out to determine the affinity of alpaca and humanized VHHs to IL-10Rα. The huE07 VHH (SEQ ID NO: 724) containing all alpaca Kabat CDRs and CDR supporting residues was found to have a modest improvement in affinity for IL-10Rα relative to the alpaca E07 due to the removal of the N-terminal AS extension. The results are summarized in Table 6. When comparing the further humanized variants, only humanization of CDR supporting residue 37 was found to dramatically reduce binding to IL-10Rα. A further humanized E07 VHH variant (996; SEQ ID NO: 725) containing alpaca Kabat CDRs plus alpaca CDR supporting residues 2, 27, 37, 45, 47, 71, and 94 (according to Kabat numbering) on the human IGHV3-23 framework was carried forward for further engineering to further increase humanness and remove primary sequence liabilities (e.g., for stability and manufacturability).

(SEQ ID NO: 725)
ELQLLESGGGLVQPGGSLRLSCAASGRTFSAYGMGWFRQAPGKGR
EFVSAIRWIGDSTYYADSVKGRFTISKDNSKNTLYLQMNSLRAED
TAVYYCAAKTSVTSYLSEKSYNYWGQGTMVTVSS

TABLE 6
Binding Characterization of Humanized E07 VHH Variants

| | | | IL-10Rα |
|---|---|---|---|
| | | | Binding |
| VHH | Grafted Alpaca CDR- | Humanized CDR- | Affinity |
| No. | Supporting Residues | Supporting Residues | KD (nM) |
| 557 | N/A | N/A | 10.0 |
| 993 | 2, 27, 37, 44, 45, 47, 49, 71, 94 | N/A | 2.5 |
| 559 | 2, 27, 37, 45, 47, 49, 71, 94 | 44 | 47.0 |
| 560 | 27, 37, 45, 47, 49, 71, 94 | 2, 44 | 12.0 |
| 561 | 2, 27, 45, 47, 49, 71, 94 | 37, 44 | 240.0 |
| 562 | 2, 27, 37, 47, 49, 71, 94 | 44, 45 | 15.0 |
| 563 | 2, 27, 37, 45, 49, 71, 94 | 44, 47 | 68.0 |
| 565 | 2, 27, 37, 45, 49, 71, 94 | 44, 71 | 44.0 |
| 994 | 2, 27, 37, 45, 47, 49, 71, 94 | 44 | 2.5 |
| 995 | 2, 27, 37, 44, 45, 47, 49, 71, 94 | 49 | 2.1 |
| 996 | 2, 27, 37, 45, 47, 71, 94 | 44, 49 | 1.6 |
| 997 | 2, 27, 44, 45, 47, 71, 94 | 37, 49 | NB* |
| 998 | 2, 27, 37, 44, 47, 71, 94 | 45, 49 | 2.0 |
| 999 | 2, 27, 37, 44, 45, 71, 94 | 47, 49 | 7.1 |

*NB = no binding detected

Sequence Liability Mitigation. A tryptophan oxidation and two isomerization primary sequence liabilities were identified in alpaca E07 VHH (SEQ ID NO: 236) at positions 52a, 55, and 61, respectively. To remove a potential tryptophan liability, amino acid substitutions W52aY and W52aN (positions according to Kabat) were introduced onto huE07 to mimic tryptophan aromaticity or hydrogen bond donor properties, respectively. To remove potential isomerization liabilities, aspartic acid at positions 55 and 61 (positions according to Kabat) was conservatively replaced with glutamic acid to maintain negative charge at those residues. Additionally, serine at positions 56 and 62 were replaced with small side-chain amino acids alanine or threonine. Surface plasmon resonance (SPR by Carterra) binding experiment was carried out to assess affinity of liability remediated variants for IL-10Rα. Both amino acid substitutions W52aY and W52aN were detrimental for IL-10Rα binding. None of the liability mitigating amino acid substitutions at positions 55, 56, 61, or 62 had any impact on IL-10Rα binding. The results are summarized in Table 7.

TABLE 7
Binding Characterization of E07 VHH Sequence
Liability Mitigation Variants

| Liability Remediation | IL-10Rα |
|---|---|
| Amino Acid | Binding Affinity |
| Substitution (Kabat) | KD (nM) |
| N/A | 10.0 |
| W52aY | 25.0 |
| W52aN | 45.8 |
| D55E | 1.7 |
| S56A | 1.2 |
| S56T | 13.6 |
| D61E | 2.0 |
| S62A | 2.3 |
| S62T | 3.8 |

An additional round of humanization and liability remediation was carried out to combine isomerization liability mitigating amino acid substitutions and further improve humanness of the humanized E07 VHH. Potential solutions for isomerization were explored by introducing the following individual amino acid substitutions, or combinations thereof, that had limited impact on IL-10Rα binding: D55E, S56A, D61E, or S62A. Humanness was increased by further humanizing positions found to have limited impact on IL-10Rα binding in the first round of humanization: 2, 44, and/or 45. SPR (Carterra) binding experiments were carried out to assess binding affinity of humanized variants to IL-10Rα. Introducing isomerization or humanizing amino acid substitutions at positions 2, 44, 45, 55, 56, 61, and 62 had little impact on binding (Table 8) and E07 VHH variant having S56A, D61E amino acid substitutions (VHH No. 1918) was chosen for affinity maturation.

TABLE 8
Binding Characterization of E07 VHH Humanization
plus Sequence Liability Mitigation Variants

| | Additional | | |
|---|---|---|---|
| | Humanizing | | IL-10Rα |
| | Amino Acid | | Binding |
| | Substitutions | Liability | Affinity |
| VHH No. | (Kabat) | Remediation | KD (nM) |
| Alpaca E07 | N/A | N/A | 10.0 |
| 1918 | 2, 44, 45 | S56A, D61E | 8.0 |
| 1920 | 2, 44, 45 | S56A, S62A | 8.0 |
| 1914 | 2, 44 | S56A, D61E | 9.5 |
| 1915 | 2, 44 | D55E, D61E | 9.9 |
| 1913 | 2, 44, 45 | N/A | 13.4 |
| 1916 | 2, 44, 45 | S56A | 13.7 |
| 1912 | 44, 45 | D55E, D61E | 14.2 |
| 1919 | 2, 44, 45 | D55E, D61E | 14.5 |
| 1917 | 2, 44, 45 | D61E | 14.5 |
| 1911 | 44, 45 | S56A, D61E | 18.1 |
| 1910 | 44, 45 | N/A | 18.6 |
| 1909 | 44 | S56A, D61E | 19.9 |

Affinity maturation. Affinity maturation of humanized anti-IL-10Rα E07 VHH having S56A, D61E amino acid substitutions was performed using yeast surface display. Sequence diversification was achieved by combinatorial, site-specific mutagenesis across the CDR regions (as defined by Kabat). Mutagenic DNA oligos were synthesized using doped nucleotide ratios, which would enable the parental E07 codon to appear at a 49.3% frequency in the library, with the remaining 63 codons appearing with a cumulative frequency of 50.7%. Following Kabat numbering, residues 31, 33, 35 in CDR1, 50, 52, 52A, 53, 55, 56 in CDR2 and 96, 97, 98, 99, 100, 100b, 100c, 100d, 100e, 100f, 101 in CDR3 were substituted. Amino acid sequences of CDR1, CDR2 and CDR3, as defined by Kabat, for E07 VHH and humanized versions thereof, are provided in Table A1. Non-CDR residue Arg 27 was allowed to mutate to Phe, which is found in the human germline, with a frequency of 50%. Similarly, Lys 78 was allowed to mutate to Arg. To enable deeper sampling of the diversity, the mutagenesis was split into two sub-libraries. One library carried mutations in FR1 to FR3, and one had mutations only in the CDR3 region.

Both sub-libraries were screened for binding to human IL-10Rα under increasingly stringent conditions. The first round of screening was performed at an antigen concentration of 50 nM IL-10Rα using magnetic-activated cell sorting (MACS) and was followed by a second round of MACS at 5 nM IL-10Rα. A third round of selections was performed using fluorescence-activated cell sorting (FACS) and a concentration of 0.5 nM IL-10Rα. The top 0.1-0.5% of the binding population was collected.

After the sub-library screening, the mutated regions were recovered via PCR and the diversity was shuffled into a final library encompassing mutations across all three CDRs. The shuffled library was screened using two rounds of MACS (50 nM and 5 nM IL-10Rα antigen concentration) and three rounds of FACS. For the third round of selection, the library was incubated with 0.5 nM IL-10Rα and allowed to reach equilibrium. For the fourth round of selection, the library was incubated with 0.1 nM antigen, allowed to reach equilibrium, and then resuspended in buffer overnight to allow for antigen dissociation. The top 1% binders were selected and correspond to variants with slower k_{off}. For the last round of selection, the library was incubated with 0.1 nM IL-10Rα for 15 min to allow for the selection of variants with higher kₒₙ, and the top 1% of binders was collected.

Following the library screening, two 96 well plates were picked, sequenced, and screened for IL-10Rα binding at 0.5 nM IL-10Rα. Unique sequences within the 75th percentile of binding to IL-10Rα were further assayed on yeast, by performing six-point binding curves on yeast and calculating EC50 values (Table 9). Twenty-seven variants were reformatted in the VHH-Fc IgG1 L234A/L235A/P331S (LALA-PS; Fc EU numbering) for recombinant expression and apparent affinity was calculated using SPR. In parallel, from the sequence analysis of the clones, thirteen constructs were synthesized in order to remove primary sequence liabilities. These thirteen variants included the R45L amino acid substitution in the VHH (according to Kabat), shown to increase the biophysical properties of the humanized E07 VHH. These were expressed as VHH-Fc IgG1 L234A/L235A/G237A (AAA; LALA-GA; Fc EU numbering) and directly assayed for IL-10Rα affinity (Table 9).

TABLE 9
Characterization Summary of Humanized E07 VHH Affinity Maturation Variants

| | | | | hIL- |
|---|---|---|---|---|
| | | | | 10Rα |
| | | | Yeast | Binding |
| | FR1 to FR3 Amino Acid | CDR3 Amino Acid Substitutions | EC₅₀ | Affinity |
| VHH Name | Substitutions (Kabat) | (Kabat) | (nM) | K_{D} (nM) |
| huR1E07 | — | — | NA | 9.5 |
| huR1E07 R45L | R45L | — | 2.1 | 7.5 |
| 1918.31 | R45L/D54V/A56G | — | 0.41 | 5.7 |
| 1918.32 | R45L/D54Y/A56G | — | 0.41 | 9.3 |
| 1918.33 | R45L/I53L/D54E/A56G | — | 0.44 | 7.4 |
| 1918.34 | R45L/W52aL/I53T/D54V/A56G | — | 0.35 | 7.5 |
| 1918.35 | R45L/D54E/A56G | — | 0.46 | 4.9 |
| 1918.36 | R27S/R45L/K71R | — | 1.05 | 14 |
| 1918.37 | R45L/I53F/A56G | — | 0.49 | 7.8 |
| 1918.5 | R45L | K100eE/N101Y | NA | 2.7 |
| 1918.8 | R45L | N101G | 2.6 | 2.7 |
| 1918.9 | R45L | S97R/T99N/K100eY/N101D | 0.15 | 11 |
| 1918.12 | R45L/I53F/A56G | V98P/N101G | 0.29 | 2.8 |
| 1918.14 | R45L/I53F/A56G | N101G | 0.32 | 7.3 |
| 1918.15 | R45L/I53Y/A56G | S97R/E100dK/K100eD/N101G | 0.24 | 5.5 |
| 1918.16 | R45L/I53Y/A56G | S97R/V98P/T99D/N101G | 0.2 | 5.9 |
| 1918.18 | R45L/I53Y/A56G | T99M/E100dK/K100eT/N101G | 0.43 | 3 |
| 1918.19 | A31P/R45L/I53W/A56G | K71R/N101Y | 1.15 | 6.1 |
| 1918.20 | R45L/I53F/D55E/A56G | T99M/E100dK/K100eS/N101G | 0.51 | 5.3 |
| 1918.21 | R45L/I53F/A56G | K100e Y/N101G | 0.29 | 8.7 |
| 1918.22 | R45L/I53Y/A56G | E100dD/K100eN/N101G | 0.43 | 5 |
| 1918.23 | R45L/I53F/A56G | T99M/N101G | 0.31 | NA |
| 1918.24 | R45L/I53Y/A56G | L100aM/E100dK/K100eE/N101Y | 0.53 | 0.702 |
| 1918.25 | R45L/I53Y/A56G | L100bM/K100eT/N101G | 0.39 | 3.2 |
| 1918.26 | R45L/I53Y/A56G | V98P/T99N/E100dA/N101T | 2.05 | 16 |
| 1918.27 | A31P/R45L/I53F/A56G | S97T/V98P/L100bM/E100dQ/N101W | 0.39 | 6.3 |
| 1918.28 | R45L/I53Y/D55E/A56G | K100eY/N101T | 0.58 | 6.4 |
| 1918.29 | R45L/I53F/D55E/A56G | S97T/T99M/E100dK/K100eN/N101G | 0.46 | 5.3 |
| 1918.30 | R45L/A56G | K100eY/N101T | 1.22 | 5.4 |
| huR1E07.1 | I53F/D55E/A56G | K100eY/N101R | NA | 4 |
| huR1E07.2 | — | K100eY/N101G | NA | 1.4 |
| huR1E07.3 | I53Y/D55E/A56G | K100eY/N101G | NA | 2 |
| huR1E07.4 | I53F/D55E/A56G | K100eY/N101G | NA | 1.6 |
| huR1E07.5 | I53Y/D55E/A56G | S100cT/E100dD/N101G | NA | 0.94 |
| huR1E07.6 | I53F/D55E/A56G | S100cT/E100dD/N101G | NA | 1 |
| huR1E07.7 | I53Y/D55E/A56G | S97R/E100dK/K100eE/N101D | NA | 1.5 |
| huR1E07.8 | I53Y/D55E/A56G | S97R/E100dK/K100eE/N101G | NA | 1.3 |
| huR1E07.9 | I53Y/D55E/A56G | E100dK/K100eE/N101Y | NA | 0.77 |
| huR1E07.10 | I53Y/D55E/A56G | V98P/N101G | NA | 1.3 |
| huR1E07.11 | I53Y/D55E/A56G | V98P/E100dV/K100eE/N101G | NA | 0.63 |
| huR1E07.12 | I53F/D55E/A56G | V98P/N101G | NA | 1.3 |
| huR1E07.13 | I53Y/D55E/A56G | S97R/V98P/T99D/N101G | NA | 0.39 |

NA = data not available

Binding on yeast revealed that most of the variants bound with lower EC₅₀ than the huR1E07 R45L parent (2.1 nM), a 2-fold to 10-fold increase in apparent affinity. Similarly, screening the recombinant proteins by SPR resulted in higher affinity relative to huR1E07 parent (9.5 nM). The highest affinity E07 VHH variant (huR1E07.13) displayed a KD value of 0.39 nM and was used for further functional characterization. An intermediate, affinity matured E07 VHH variant (huR1E07.12) displayed a KD value of 1.3 nM was also included in downstream assays.

### Anti-IL-10Rβ A05 VHH Engineering

Humanization. The humanness of the alpaca A05 VHH (SEQ ID NO: 450; Table D) was assessed using BioPhi (Prihoda, et al., supra). The alpaca CDRs were extracted using the Kabat definition (CDR1: residues 31-35, CDR2: residues 50-65, CDR3: residues 95-102) and grafted on the highly homologous human IGHV3-23*01 and IGHJ3*01 germline frameworks. Framework residues in positions 2, 27-30, 47-49, 78, 94, 95 (according to Kabat) were substituted to the original alpaca amino acids where applicable, as these positions have been shown to support the proper orientation of the CDR loops (Foote, et al., J Mol Biol (1992) 224(2):487-99). Furthermore, positions 37 and 45 in the framework region 2 were also substituted to the original alpaca amino acids (i.e., “back-mutated”), as these residues influence stability and solubility of the variable domain, when not paired with a light chain (Vincke, et al., J Biol Chem (2009) 284(5):3273-3284). The resulting amino acid sequence huA05 (SEQ ID NO: 726) has the highest content of non-human residues and was used as a template for further humanization.

(SEQ ID NO: 726)
EVQLVESGGGLVQPGGSLRLSCAASGNSLDYPTIGWFRQAPGKGR
EGVSCISASDGSTHYADSVKGRFTISRDNSKNTVYLQMNSLRAED
TAVYYCAASLWGLGCKSEADYDYWGQGTQVTVSS

Humanizing amino acid substitutions at positions 37, 45, 47 and 78 were individually added and the resulting molecules were expressed recombinantly and tested for binding via SPR and for thermal stability via differential scanning fluorimetry. Only the R45L and V78L-containing variants maintained binding to IL-10Rβ (Table 10), and were carried forward, both as individual and combined amino acid substitutions.

TABLE 10
Characterization Summary of A05 VHH Variants

| | Human | | | | |
|---|---|---|---|---|---|
| | Amino | Liability | IL-10Rβ | | |
| | Acid | Amino Acid | Binding | | |
| VHH | Substitutions | Substitutions | Affinity | | Tₒₙₛₑₜ |
| Name | (Kabat) | (Kabat) | K_{D} (nM) | T_{M}1 (° C.) | (° C.) |
| A05 | — | — | 9 | 62.5 (n = 3) | 58.6 (n = 2) |
| hu.A05.1 | — | — | 13.1 | 65.1 | NA |
| hu.A05.2 | F37V | — | NB | 64.8 | NA |
| hu.A05.3 | R45L | — | 7.8 | 65.1 | NA |
| hu.A05.4 | G47W | — | NB | NA | NA |
| hu.A05.5 | V78L | — | 10.8 | 64.5 | NA |
| | R45L/V78L | — | 13.9 | NA | NA |
| A05_SS | — | C50S/C100aS | NB | 64.5 | NA |
| A05_AA | — | C50A/C100aA | 210 | 62.4 (n = 2) | 56.7 |
| A05_SA | — | C50S/C100aA | NB | 63.8 | NA |
| A05_AS | — | C50A/C100aS | NB | 64.2 | NA |
| BD-878 | R45L | G55A | 7.28 | NA | NA |
| BD-873 | R45L | D61E | 6.44 | NA | NA |
| BD-1221 | R45L | S62A | 8.4 | NA | NA |
| BD-900 | R45L | W97H | 7.99 | NA | NA |
| BD-1222 | R45L/V78L | G55A | 14.8 | NA | NA |
| BD-1223 | R45L/V78L | D61E | 12.6 | NA | NA |
| BD-1224 | R45L/V78L | S62A | 11.3 | NA | NA |
| BD-1225 | R45L/V78L | W97H | 5.65 | NA | NA |
| BD-1230 | R45L | C50A/C100aA/G55A/W97H | 240 | 59.4 | 54.4 |
| BD-1232 | R45L | C50A/C100aA/W97H | 335 | NA | NA |
| BD-1227 | R45L/V78L | G55A/D61E | 14.8 | NA | NA |
| BD-1228 | R45L/V78L | G55A/S62A | 11.9 | 57.8 | 53.7 |
| BD-1229 | R45L/V78L | G55A/W97H | 6.19 | NA | NA |
| BD-1215 | R45L/V78L | G55A/D61E/W97H | 6.08 | NA | NA |
| BD-1216 | R45L/V78L | G55A/S62A/W97H | 5.59 | 59.9 | 53.6 |
| BD-1217 | R45L/V78L | C50A/C100aA | 240 | NA | NA |
| BD-1218 | R45L/V78L | C50A/C100a/G55A | 332 | NA | NA |
| BD-1219 | R45L/V78L | C50A/C100aA/W97H | 154 | NA | NA |
| BD-1220 | R45L/V78L | C50A/C100aA/G55A/W97H | 196 | 57.0 | 52.5 |
| | R45L/Q108M | C50A/C100aA/G55A/S62A/W97H | 202 | 59.5 | 54.5 |

*NB = no binding detected, NA = data not available

Sequence Liability Mitigation. The CDRs of alpaca A05 VHH (SEQ ID NO: 450) contain putative biophysical liabilities that could impact the developability of the molecule. These include two aspartate (Asp) isomerization sites at positions 54 and 61, one tryptophan (Trp) oxidation site at position 93 and a non-canonical disulfide bond between Cys50 and Cys100a (Position numbering according to Kabat). We engineered out these liabilities by substituting these amino acid residues to amino acids with similar biophysical properties. Residues directly adjacent to the isomerization sites were also substituted. These amino acid substitutions were included in a humanized variant of A05 VHH huA05 (SEQ ID NO: 726; huA05.3). Certain disulfide-removal amino acid substitutions were also tested on the alpaca backbone. The complete set of molecules (Table 10) was tested for binding and a subset of those were also tested for thermal stability. Liability remediation amino acid substitutions across two, three or all four sites were combined and tested as well.

The amino acid substitutions tested to mitigate the Asp isomerization and the Trp oxidation had little impact on binding. Combinations of these amino acid substitutions also fell within a two-fold affinity window of the parent alpaca A05 VHH. Of the pair of amino acid substitutions tested for the removal of the non-canonical disulfide bond, only the C50A/C100aA substitutions resulted in detectable binding, albeit at 20- to 30-fold lower levels than the parent alpaca A05 VHH. Combining R45L and V78L humanizing amino acid substitutions resulted in slight but detectable loss of binding and a lower Tₒₙₛₑₜ value, over R45L alone, so the V78L amino acid substitution was removed from the final molecules. The final molecule, which contained the R45L humanizing amino acid substitution and the C50A/C100aA/G55A/S62A/W97H liability removal amino acid substitutions, also included the Q108M amino acid substitution in the FR4 and bound to the target with an affinity of 202 nM (Table 10, bottom row; SEQ ID NO: 727).

(SEQ ID NO: 727)
EVQLVESGGGLVQPGGSLRLSCAASGNSLDYPTIGWFRQAPGKGL
EGVSAISASDASTHYADAVKGRFTISRDNSKNTVYLQMNSLRAED
TAVYYCAASLHGLGAKSEADYDYWGQGTMVTVSS

Affinity Detuning. To obtain a panel of variable IL-10Rβ affinities, additional amino acid substitutions were introduced in the CDR regions of the A05 (SEQ ID NO: 728).

(SEQ ID NO: 728)
EVQLVESGGGLVQPGGSLRLSCAASGNSLDYPTIGWFRQAPGKGL
EGVSCISASDGSTHYADSVKGRFTISRDNSKNTVYLQMNSLRAED
TAVYYCAASLWGLGCKSEADYDYWGQGTQVTVSS

Since the removal of the non-canonical disulfide bond resulted in dramatic loss of affinity, the detuning amino acid substitutions were introduced in the presence of the disulfide bond. A panel of variants was designed where each variant contained one amino acid substitution. The amino acid substitutions were selected to have similar or divergent biophysical properties from the original alpaca amino acids. These amino acid substitutions were grafted on the humanized A05 (SEQ ID NO: 728) and a variant that contained the amino acid substitutions D54E and S62A at the sites of two Asp isomerization residues, respectively (Table 11).

TABLE 11
Characterization Summary of Humanized A05 VHH Affinity Detuned Variants

| | IL-10Rβ | Binding | | |
|---|---|---|---|---|
| | Binding | Affinity | | |
| Amino Acid Substitutions | Affinity | Fold- | T_{M}1 | Tonset |
| (Kabat) | K_{D} (nM) | reduction | (° C.) | (° C.) |
| — | 9 | — | 62.5 (n = 3) | 58.6 (n = 2) |
| A100eD | 81 | 9.0 | NA | NA |
| A100eK | 68.6 | 7.6 | NA | NA |
| A100eS | 10.7 | 1.2 | NA | NA |
| A52aS | 12.2 | 1.4 | NA | NA |
| D100fA | NA | NA | NA | NA |
| D100fE | 620 | 68.9 | NA | NA |
| D100fK | NA | NA | NA | NA |
| D101A | 119 | 13.2 | NA | NA |
| D101E | 8.66 | 1.0 | NA | NA |
| D101K | 203 | 22.6 | NA | NA |
| D54R | 18.2 | 2.0 | NA | NA |
| D61E | 6.35 | 0.7 | NA | NA |
| E100dA | 9.39 | 1.0 | NA | NA |
| E100dD | 12 | 1.3 | NA | NA |
| E100dS | 20.5 | 2.3 | NA | NA |
| G100S | 431 | 47.9 | NA | NA |
| G55A | 12.3 | 1.4 | NA | NA |
| G98S | 482 | 53.6 | NA | NA |
| I34G | 28.5 | 3.2 | NA | NA |
| K100bE | 1310 | 145.6 | NA | NA |
| K100bR | 14.5 | 1.6 | NA | NA |
| L96G | 37.1 | 4.1 | NA | NA |
| L96S | 17.3 | 1.9 | NA | NA |
| L99A | 75.8 | 8.4 | 60.18 | 55.2 |
| L99G | 275 | 30.6 | NA | NA |
| P32A | 80.8 | 8.9 | NA | NA |
| P32G | 53.7 | 6.0 | NA | NA |
| P32S | 123 | 13.7 | NA | NA |
| P32Y | 82.9 | 9.2 | NA | NA |
| S100cA | 10.4 | 1.2 | NA | NA |
| S100cL | NA | NA | NA | NA |
| S53G | 11.3 | 1.3 | NA | NA |
| L95R | NA | NA | NA | NA |
| L95T | 242 | 26.9 | NA | NA |
| T33A | 21.7 | 2.4 | NA | NA |
| T33N | 18.9 | 2.1 | NA | NA |
| T33S | 36.4 | 4.0 | NA | NA |
| W97F | 10.7 | 1.2 | NA | NA |
| W97H | 4.84 | 0.5 | NA | NA |
| W97L | 11.2 | 1.2 | NA | NA |
| W97Y | 8 | 0.9 | NA | NA |
| Y100gF | 43 | 4.8 | NA | NA |
| Y100gH | 459 | 51.0 | NA | NA |
| Y100gT | NA | NA | NA | NA |
| W102F | 6.93 | 0.8 | NA | NA |
| D54E/S62A | 15.2 | 1.7 | NA | NA |
| D54E/S62A/A100eD | 152 | 16.9 | NA | NA |
| D54E/S62A/A100eK | 135 | 15.0 | NA | NA |
| D54E/S62A/A100eS | 28.2 | 3.1 | NA | NA |
| D54E/S62A/A52aS | 20.6 | 2.3 | 58.93 | 54.5 |
| D54E/S62A/D100fA | NA | NA | NA | NA |
| D54E/S62A/D100fK | NA | NA | NA | NA |
| D54E/S62A/D101A | 220 | 24.4 | NA | NA |
| D54E/S62A/D101E | 12.6 | 1.4 | NA | NA |
| D54E/S62A/D101K | 477 | 53.0 | NA | NA |
| D54R/S62A | 15.9 | 1.8 | NA | NA |
| D54E/S62A/D61E | 13 | 1.4 | NA | NA |
| D54E/S62A/E100dA | 25.1 | 2.8 | NA | NA |
| D54E/S62A/E100dD | 25 | 2.8 | NA | NA |
| D54E/S62A/E100dS | 19.2 | 2.1 | NA | NA |
| D54E/S62A/G100S | NA | NA | NA | NA |
| D54E/S62A/G55A | 12.7 | 1.4 | NA | NA |
| D54E/S62A/G98S | 25.9 | 2.9 | NA | NA |
| D54E/S62A/I34G | NA | NA | NA | NA |
| D54E/S62A/K100bE | 473 | 52.6 | NA | NA |
| D54E/S62A/K100bR | 29.8 | 3.3 | NA | NA |
| D54E/S62A/L96G | 69.1 | 7.7 | NA | NA |
| D54E/S62A/L96S | 26.1 | 2.9 | NA | NA |
| D54E/S62A/L99A | 138 | 15.3 | NA | NA |
| D54E/S62A/L99S | 587 | 65.2 | NA | NA |
| D54E/S62A/P32A | 109 | 12.1 | NA | NA |
| D54E/S62A/P32G | 80.3 | 8.9 | 59.61 | 54.9 |
| D54E/S62A/P32S | 180 | 20.0 | 59.84 | 54.9 |
| D54E/S62A/P32Y | 156 | 17.3 | 59.74 | 55.1 |
| D54E/S62A/S100cA | 145 | 1.6 | NA | NA |
| D54E/S62A/S100cL | NB | NB | NA | NA |
| D54E/S62A/S53G | 191 | 2.1 | NA | NA |
| D54E/S62A/L95R | NB | NB | NA | NA |
| D54E/S62A/L95T | 473 | 52.6 | NA | NA |
| D54E/S62A/T33A | 30.6 | 3.4 | 58.36 | 55.4 |
| D54E/S62A/T33N | 47.3 | 5.3 | NA | NA |
| D54E/S62A/T33S | 74.8 | 8.3 | 58.59 | 54.6 |
| D54E/S62A/W97F | 19.9 | 2.2 | NA | NA |
| D54E/S62A/W97H | 6.58 | 0.7 | 60.64 | 55.2 |
| D54E/S62A/W97L | 16.4 | 1.8 | NA | NA |
| D54E/S62A/W97Y | 20.3 | 2.3 | NA | NA |
| D54E/S62A/Y100gF | 91.4 | 10.2 | 59.45 | 54.7 |
| D54E/S62A/Y100gH | 918 | 102.0 | NA | NA |
| D54E/S62A/Y100gT | NB | NB | NA | NA |
| D54E/S62A/W102F | 13.8 | 1.5 | 59.02 | 55.0 |
| D54E/S62A/W97H/Q108M | NA | NA | NA | NA |
| D54E/S62A/W97H/L99A/Q108M | NA | NA | NA | NA |
| D54E/S62A/L96G/W97H/Q108M | 15.4 | 1.7 | 60.64 | 55.5 |
| D54E/S62A/P32S/W97H/Q108M | 122 | 13.6 | 60.62 | 55.2 |

The tested variants displayed a broad range of apparent KD values, ranging from 0.5 to 150-fold reduced affinities. A few amino acid substitutions, primarily in CDR3, resulted in completely abolished binding to IL-10Rβ. Overall, variants including the deamidation-correcting amino acid substitutions had lower affinity. However, there were numerous A05 VHH variants that displayed affinities close to the A05 VHH alpaca parent. Ten variants, primarily from the triple amino acid substitution group, having affinities ranging from 6.5 nM to 180 nM, were selected for additional thermal stability assays. The resulting TM1 values were lower than the alpaca parent A05 VHH, but most were within 2° C. difference. Variants with TM1<60° C. were de-prioritized. Three amino acid substitutions (P32S, L96A, L99A) were combined with the oxidation-removing amino acid substitution W97H and the humanizing amino acid substitution Q108M, and formatted with the anti-IL-10Rα in tandem for functional characterization.

N-terminal AS Motif Engineering. A small panel of tandem VHH1-VHH2-Fc molecules was designed such that VHH1 was an anti-IL-10Rα and VHH2 was an anti-IL-10Rβ binder to understand the impact of the N-terminal alanine+serine (AS) extension on IL-10 receptor agonism. The VHH1 anti-IL-10Rα binder composed of either parent alpaca E07 VHH (SEQ ID NO: 236) or huE07 VHH SEQ ID NO: 729 plus/minus the N-terminal AS extension and VHH2 anti-IL-10Rβ binder was composed of either parent alpaca A05 VHH (SEQ ID NO: 450) or huA05 VHH (SEQ ID NO: 728; huA05.3) plus/minus the N-terminal AS extension (Table 12).

(SEQ ID NO: 729)
EVQLLESGGGLVQPGGSLRLSCAASGRTFSAYGMGWFRQAPGKGR
EFVSAIRWIGDATYYAESVKGRFTISKDNSKNTLYLQMNSLRAED
TAVYYCAAKTSVTSYLSEKSYNYWGQGTMVTVSS

TABLE 12
Functional Characterization of N-Terminal AS Motif in
Alpaca or Humanized E07-A05 Tandem VHH

| | | | HEK IL-10R | |
|---|---|---|---|---|
| | | | Reporter Assay | |
| VHH1-VHH2 | | VHH1-VHH2 | EC50 | Emax |
| Molecule | Description | Amino Acid Sequence | (nM) | (%) |
| 768 | Alpaca E07-A05 | SEQ ID NO: 730 | 1.24 | 32.9 |
| | VHH | QLQLVESGGGLVRAGGSLRLSCAASGRIFSAY | | |
| | | GMGWFRQAPGKEREFVAAIRWIGDSTYYADSV | | |
| | | KGRFTISKDNAKNTLYLQMSSLKPEDTAVYYC | | |
| | | AAKTSVTSYLSEKSYNYWGQGTQVTVSSGGGS | | |
| | | QVQLVESGGGLVQPGGSLRLSCAASGNSLDYP | | |
| | | TIGWFRQAPGKEREGVSCISASDGSTHYADSV | | |
| | | KGRFTISRDNTKNTVDLQMNNLKPEDTGHYYC | | |
| | | AASLWGLGCKSEADYDYWGQGTQVTVSS | | |
| 087 | N-AS + Alpaca | SEQ ID NO: 731 | 1.08 | 54.65 |
| | E07-A05 VHH | ASQLQLVESGGGLVRAGGSLRLSCAASGRTFS | | |
| | | AYGMGWFRQAPGKEREFVAAIRWIGDSTYYAD | | |
| | | SVKGRFTISKDNAKNTLYLQMSSLKPEDTAVY | | |
| | | YCAAKTSVTSYLSEKSYNYWGQGTQVTVSSGG | | |
| | | GSQVQLVESGGGLVQPGGSLRLSCAASGNSLD | | |
| | | YPTIGWFRQAPGKEREGVSCISASDGSTHYAD | | |
| | | SVKGRFTISRDNTKNTVDLQMNNLKPEDTGHY | | |
| | | YCAASLWGLGCKSEADYDYWGQGTQVTVSS | | |
| 299 | Humanized E07-A05 | SEQ ID NO: 732 | 2.28 | 31.45 |
| | VHH | EVQLLESGGGLVQPGGSLRLSCAASGRTFSAY | | |
| | | GMGWFRQAPGKGREFVSAIRWIGDATYYAESV | | |
| | | KGRFTISKDNSKNTLYLQMNSLRAEDTAVYYC | | |
| | | AAKTSVTSYLSEKSYNYWGQGTMVTVSSGGGS | | |
| | | EVQLVESGGGLVQPGGSLRLSCAASGNSLDYP | | |
| | | TIGWFRQAPGKGLEGVSCISASDGSTHYADSV | | |
| | | KGRFTISRDNSKNTVYLQMNSLRAEDTAVYYC | | |
| | | AASLWGLGCKSEADYDYWGQGTQVTVSS | | |
| 294 | N-AS + humanized | SEQ ID NO: 733 | 1.79 | 13.4 |
| | E07-A05 VHH | ASEVQLLESGGGLVQPGGSLRLSCAASGRIES | | |
| | | AYGMGWFRQAPGKGREFVSAIRWIGDATYYAE | | |
| | | SVKGRFTISKDNSKNTLYLQMNSLRAEDTAVY | | |
| | | YCAAKTSVTSYLSEKSYNYWGQGTMVTVSSGG | | |
| | | GSEVQLVESGGGLVQPGGSLRLSCAASGNSLD | | |
| | | YPTIGWFRQAPGKGLEGVSCISASDGSTHYAD | | |
| | | SVKGRFTISRDNSKNTVYLQMNSLRAEDTAVY | | |
| | | YCAASLWGLGCKSEADYDYWGQGTQVTVSS | | |

The tandem VHH1-VHH2 molecules were tested in a HEK IL-10 receptor reporter assay and all four molecules had comparable EC50 values in the reporter assay, suggesting the N-terminal AS does not impact potency. The fully alpaca VHH1-VHH2 molecule 768 containing the N-terminal AS (SEQ ID NO: 731) had the highest Eₘₐₓ of 54.65%. Removal of the N-terminal AS (molecule 087; SEQ ID NO: 730) resulted in a reduction in Emax by about 20%, suggesting that the N-terminal AS impacts Emax. The humanized tandem VHH1-VHH2 molecule (molecule 299; SEQ ID NO: 732) had a reduced Emax of 31.45% relative to the fully alpaca molecule containing the N-terminal AS. Addition of the N-terminal AS extension onto the humanized molecule (molecule 294; SEQ ID NO: 733) further reduced the Emax to 13.4%.

The homology model of the alpaca E07 VHH (SEQ ID NO: 236) was further analyzed in Pymol to map all amino acids within 5 Å of residue Q1. The only residues that differed between huE07 VHH molecule 294 (SEQ ID NO: 733) and alpaca parent E07 VHH (SEQ ID NO: 236) were residues 1 and 2 (according to Kabat) and these residues were hypothesized to be the cause of Emax loss observed during humanization. Three additional tandem VHH1-VHH2 molecules were designed with modifications to the N-terminal residues. In one molecule, the human amino acid as position 1 was substituted back to the alpaca amino acid (E1Q) (SEQ ID NO: 734). In another molecule, both the human amino acids at position 1 and 2 were substituted back to the alpaca amino acids (E1Q+V2L) (SEQ ID NO: 735). In a third molecule, the N-terminal AS extension was removed, residue 1 was substituted from glutamic acid to glycine, and residue 2 was substituted from valine to leucine (SEQ ID NO: 736). The three engineered molecules plus the N-terminal AS extended alpaca parent molecule were tested in HEK reporter assay. The molecule containing backmutations (i.e., reverted amino acid substitutions) at residues 1 and 2 from human to alpaca amino acids was sufficient to increase Eₘₐₓ to a level comparable to the parental alpaca molecule. All further engineered molecules contain the N-terminal AS extension with Q1 and L2 (e.g. “ASQL” at positions 1 to 4). The results are summarized in Table 13.

(SEQ ID NO: 734)
ASQVQLLESGGGLVQPGGSLRLSCAASGRTFSAYGMGWFRQAPGK
GREFVSAIRWIGDATYYAESVKGRFTISKDNSKNTLYLQMNSLRA
EDTAVYYCAAKTSVTSYLSEKSYNYWGQGTMVTVSSGGGSEVQLV
ESGGGLVQPGGSLRLSCAASGNSLDYPTIGWFRQAPGKGLEGVSC
ISASDGSTHYADSVKGRFTISRDNSKNTVYLQMNSLRAEDTAVYY
CAASLWGLGCKSEADYDYWGQGTQVTVSS
(SEQ ID NO: 735)
ASQLQLLESGGGLVQPGGSLRLSCAASGRTFSAYGMGWFRQAPGK
GREFVSAIRWIGDATYYAESVKGRFTISKDNSKNTLYLQMNSLRA
EDTAVYYCAAKTSVTSYLSEKSYNYWGQGTMVTVSSGGGSEVQLV
ESGGGLVQPGGSLRLSCAASGNSLDYPTIGWFRQAPGKGLEGVSC
ISASDGSTHYADSVKGRFTISRDNSKNTVYLQMNSLRAEDTAVYY
CAASLWGLGCKSEADYDYWGQGTQVTVSS
(SEQ ID NO: 736)
GLQLVESGGGLVQPGGSLRLSCAASGRTFSAYGMGWERQAPGKGR
EFVAAIRWIGDSTYYADSVKGRFTISKDNSKNTLYLQMNSLRAED
TAVYYCAAKTSVTSYLSEKSYNYWGQGTLVTVSSGGGSEVQLVES
GGGLVQPGGSLRLSCAASGNSLDYPTIGWFRQAPGKGLEGVSCIS
ASDGSTHYADSVKGRFTISRDNSKNTVYLQMNSLRAEDTAVYYCA
ASLWGLGCKSEADYDYWGQGTQVTVSS

TABLE 13
Functional Characterization of N-terminal
AS Motif Engineering Variants

| VHH1-VHH2 | SEQ ID | Description | |
|---|---|---|---|
| Molecule | NO: | N-terminal Residues | Emax (%) |
| 087 | 731 | AS + alpaca VHH | 74.90 |
| 252 | 734 | AS + E1Q | 49.00 |
| 253 | 735 | AS + E1Q + V2L | 71.50 |
| 254 | 736 | E1G + V2L | 50.75 |

Humanized E07-A05 Tandem VHH1-VHH2 Affinity Panel. Three affinity matured E07 VHH and five affinity detuned were combined into a final panel of tandem VHH1-VHH2-Fc molecules (Table 14; substituted amino acid residues shown in bolded text). All the E07 VHH contained the ASQL N-terminal extension. The linker between the VHH1 and VHH2 domains was a Gly-Gly-Gly-Ser (GGGS; SEQ ID NO: 715), and the linker between the C-terminal anti-IL-10Rβ VHH and the Fc was Gly-Ser (GS). The Fc used for this panel was a silenced human IgG1 isotype, containing the L234A/L235A/G237A amino acid substitutions (Hale, et al., MAbs (2024) 16(1):2402701).

TABLE 14
Characterization Summary of Humanized VHH1-VHH2 E07-A05 Tandem VHH1-VHH2 Affinity Panel

| | hIL-10Rα | hIL-10Rβ | HEK | HEK |
|---|---|---|---|---|
| | Binding | Binding | reporter | reporter |
| | Affinity | Affinity | EC50 | (Emax |
| E07-A05 Tandem VHH1-VHH2 Amino Acid Sequence | (nM) | (nM) | (nM) | %) |
| SEQ ID NO: 737 | 55 | 6.3 | 1.54 | 69 |
| ASQLQLLESGGGLVQPGGSLRLSCAASGRIFSAYGMGWFRQAPGKGREFVSAIR | | | | |
| WIGDATYYAESVKGRFTISKDNSKNTLYLQMNSLRAEDTAVYYCAAKTSVTSYL | | | | |
| SEKSYNYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASLHGLGCKSEADYDYWGQGTMVTVSS | | | | |
| SEQ ID NO: 738 | 55 | 15.4 | 3.10 | 63.1 |
| ASQLQLLESGGGLVQPGGSLRLSCAASGRIFSAYGMGWFRQAPGKGREFVSAIR | | | | |
| WIGDATYYAESVKGRFTISKDNSKNTLYLQMNSLRAEDTAVYYCAAKTSVTSYL | | | | |
| SEKSYNYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASGHGLGCKSEADYDYWGQGTMVTVSS | | | | |
| SEQ ID NO: 739 | 55 | 70.1 | 1.11 | 78 |
| ASQLQLLESGGGLVQPGGSLRLSCAASGRTFSAYGMGWFRQAPGKGREFVSAIR | | | | |
| WIGDATYYAESVKGRFTISKDNSKNTLYLQMNSLRAEDTAVYYCAAKTSVTSYL | | | | |
| SEKSYNYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASLHGAGCKSEADYDYWGQGTMVTVSS | | | | |
| SEQ ID NO: 740 | 55 | 122 | 3.40 | 64.3 |
| ASQLQLLESGGGLVQPGGSLRLSCAASGRTFSAYGMGWFRQAPGKGREFVSAIR | | | | |
| WIGDATYYAESVKGRFTISKDNSKNTLYLQMNSLRAEDTAVYYCAAKTSVTSYL | | | | |
| SEKSYNYWGQGTMVTVSSGGGSEVQLVESGGGLVQPGGSLRLSCAASGNSLDYS | | | | |
| TIGWFRQAPGKGLEGVSCISASEGSTHYADAVKGRFTISRDNSKNTVYLQMNSL | | | | |
| RAEDTAVYYCAASLHGLGCKSEADYDYWGQGTMVTVSS | | | | |
| SEQ ID NO: 741 | 55 | 201 | 4.96 | 52.5 |
| ASQLQLLESGGGLVQPGGSLRLSCAASGRTFSAYGMGWFRQAPGKGREFVSAIR | | | | |
| WIGDATYYAESVKGRFTISKDNSKNTLYLQMNSLRAEDTAVYYCAAKTSVTSYL | | | | |
| SEKSYNYWGQGTMVTVSSGGGSEVQLVESGGGLVQPGGSLRLSCAASGNSLDYP | | | | |
| TIGWFRQAPGKGLEGVSAISASDASTHYADAVKGRFTISRDNSKNTVYLQMNSL | | | | |
| RAEDTAVYYCAASLHGLGAKSEADYDYWGQGTMVTVSS | | | | |
| SEQ ID NO: 742 | 25.7 | 6.3 | 3.39 | 73 |
| ASQLQLLESGGGLVQPGGSLRLSCAASGRTFSAYGMGWFRQAPGKGREFVSAIR | | | | |
| WFGEGTYYAESVKGRFTISKDNSKNTLYLQMNSLRAEDTAVYYCAAKTSPTSYL | | | | |
| SEKSYGYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASLHGLGCKSEADYDYWGQGTMVTVSS | | | | |
| SEQ ID NO: 743 | 25.7 | 15.4 | 0.83 | 64 |
| ASQLQLLESGGGLVQPGGSLRLSCAASGRIFSAYGMGWFRQAPGKGREFVSAIR | | | | |
| WFGEGTYYAESVKGRFTISKDNSKNTLYLQMNSLRAEDTAVYYCAAKTSPTSYL | | | | |
| SEKSYGYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASGHGLGCKSEADYDYWGQGTMVTVSS | | | | |
| SEQ ID NO: 744 | 25.7 | 70.1 | 2.05 | 76.9 |
| ASQLQLLESGGGLVQPGGSLRLSCAASGRTFSAYGMGWFRQAPGKGREFVSAIR | | | | |
| WFGEGTYYAESVKGRFTISKDNSKNTLYLQMNSLRAEDTAVYYCAAKTSPTSYL | | | | |
| SEKSYGYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASLHGAGCKSEADYDYWGQGTMVTVSS | | | | |
| SEQ ID NO: 745 | 25.7 | 122 | 3.43 | 78.5 |
| ASQLQLLESGGGLVQPGGSLRLSCAASGRTFSAYGMGWFRQAPGKGREFVSAIR | | | | |
| WFGEGTYYAESVKGRFTISKDNSKNTLYLQMNSLRAEDTAVYYCAAKTSPTSYL | | | | |
| SEKSYGYWGQGTMVTVSSGGGSEVQLVESGGGLVQPGGSLRLSCAASGNSLDYS | | | | |
| TIGWFRQAPGKGLEGVSCISASEGSTHYADAVKGRFTISRDNSKNTVYLQMNSL | | | | |
| RAEDTAVYYCAASLHGLGCKSEADYDYWGQGTMVTVSS | | | | |
| SEQ ID NO: 746 | 25.7 | 201 | 3.05 | 58 |
| ASQLQLLESGGGLVQPGGSLRLSCAASGRTFSAYGMGWFRQAPGKGREFVSAIR | | | | |
| WFGEGTYYAESVKGRFTISKDNSKNTLYLQMNSLRAEDTAVYYCAAKTSPTSYL | | | | |
| SEKSYGYWGQGTMVTVSSGGGSEVQLVESGGGLVQPGGSLRLSCAASGNSLDYP | | | | |
| TIGWFRQAPGKGLEGVSAISASDASTHYADAVKGRFTISRDNSKNTVYLQMNSL | | | | |
| RAEDTAVYYCAASLHGLGAKSEADYDYWGQGTMVTVSS | | | | |
| SEQ ID NO: 747 | 12.5 | 6.3 | 3.05 | 71.5 |
| ASQLQLLESGGGLVQPGGSLRLSCAASGRTFSAYGMGWERQAPGKGREFVSAIR | | | | |
| WYGEGTYYAESVKGRFTISKDNSKNTLYLQMNSLRAEDTAVYYCAAKTRPDSYL | | | | |
| SEKSYGYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASLHGLGCKSEADYDYWGQGTMVTVSS | | | | |
| SEQ ID NO: 748 | 12.5 | 15.4 | 1.26 | 74 |
| ASQLQLLESGGGLVQPGGSLRLSCAASGRTFSAYGMGWFRQAPGKGREFVSAIR | | | | |
| WYGEGTYYAESVKGRFTISKDNSKNTLYLQMNSLRAEDTAVYYCAAKTRPDSYL | | | | |
| SEKSYGYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASGHGLGCKSEADYDYWGQGTMVTVSS | | | | |
| SEQ ID NO: 749 | 12.5 | 70.1 | 0.47 | 80 |
| ASQLQLLESGGGLVQPGGSLRLSCAASGRTFSAYGMGWFRQAPGKGREFVSAIR | | | | |
| WYGEGTYYAESVKGRFTISKDNSKNTLYLQMNSLRAEDTAVYYCAAKTRPDSYL | | | | |
| SEKSYGYWGQGTMVTVSSGGGSEVQLVESGGGLVQPGGSLRLSCAASGNSLDYP | | | | |
| TIGWFRQAPGKGLEGVSCISASEGSTHYADAVKGRFTISRDNSKNTVYLQMNSL | | | | |
| RAEDTAVYYCAASLHGAGCKSEADYDYWGQGTMVTVSS | | | | |
| SEQ ID NO: 750 | 12.5 | 122 | 1.74 | 75 |
| ASQLQLLESGGGLVQPGGSLRLSCAASGRTFSAYGMGWFRQAPGKGREFVSAIR | | | | |
| WYGEGTYYAESVKGRFTISKDNSKNTLYLQMNSLRAEDTAVYYCAAKTRPDSYL | | | | |
| SEKSYGYWGQGTMVTVSSGGGSEVQLVESGGGLVQPGGSLRLSCAASGNSLDYS | | | | |
| TIGWFRQAPGKGLEGVSCISASEGSTHYADAVKGRFTISRDNSKNTVYLQMNSL | | | | |
| RAEDTAVYYCAASLHGLGCKSEADYDYWGQGTMVTVSS | | | | |
| SEQ ID NO: 751 | 12.5 | 201 | 1.01 | 60 |
| ASQLQLLESGGGLVQPGGSLRLSCAASGRIFSAYGMGWFRQAPGKGREFVSAIR | | | | |
| WYGEGTYYAESVKGRFTISKDNSKNTLYLQMNSLRAEDTAVYYCAAKTRPDSYL | | | | |
| SEKSYGYWGQGTMVTVSSGGGSEVQLVESGGGLVQPGGSLRLSCAASGNSLDYP | | | | |
| TIGWFRQAPGKGLEGVSAISASDASTHYADAVKGRFTISRDNSKNTVYLQMNSL | | | | |
| RAEDTAVYYCAASLHGLGAKSEADYDYWGQGTMVTVSS | | | | |

### Anti-hIL-10Rα VHH (F11) Engineering

Humanization. The humanness of alpaca F11 VHH (SEQ ID NO: 250) was assessed using BioPhi (Prihoda, et al., supra). The alpaca VHH was found to contain 62% sequence similarity to human framework IGHV3-23 and human IGHV3-23 was chosen as the acceptor framework. Homology models of alpaca F11 VHH (SEQ ID NO: 250) and the human IGHV3-23*01 germline sequence were generated using ImmuneBuilder (Abanades, et al., supra) and viewed in PyMol (pymol.org). All residues within 5 Å of Kabat CDRs were defined as “CDR-supporting” residues and were considered for grafting from alpaca framework, in addition to Kabat CDRs, onto the human acceptor framework. All residues that differ between alpaca and human that were greater than 5 Å away from Kabat CDRs were left human. Three humanized variants of alpaca F11 VHH were generated with increasing similarity to the human germline sequence (Table 15). The generated homology models were analyzed in order to determine which residues CDR-supporting residues that differ between alpaca and human should be grafted onto the acceptor human framework, IGHV3-23*01. The amino acid positions are according to Kabat numbering. The first humanized VHH 3959, was generated by grafting all alpaca Kabat CDRs and CDR supporting residues onto the human framework, except solvent exposed amino acid 83. A more human-like variant, VHH 4008, was generated by substituting amino acid residues 2, 49, and 73 of VHH 3959 to the human amino acid. Finally, the third and most human-like variant was generated by grafting only the alpaca Kabat CDRs, but not the alpaca CDR-supporting residues, onto the human acceptor framework. Two of the humanized variants, VHH 4007 and VHH 4008, were expressed with a titer >250 mg/L, but the most human-like variant, VHH 3959, could not be expressed. Between the two expressed variants, the VHH 4008 variant had depressed DSF measured melting temperature (TMs) relative to VHH 4007. Consistent with the reduced thermostability, >70% high molecular weight species were observed for the VHH 4007 variant after 1 week at 50° C. The VHH 4008 humanized variant had no appreciable increase in aggregation after 1 week at 50° C.

TABLE 15
Characterization Summary of Humanized VHH F11 Variants

| | | | | | | | | | aSEC, | |
|---|---|---|---|---|---|---|---|---|---|---|
| | | | | | | | aSEC, T0 | | 50° C. 1 week | |
| | | Grafted Alpaca CDR- | Expression | DSF (Uncle) | | | | % | | % |
| VHH | clone | Supporting Residues | Titer | Tonset | TM1 | TM2 | % | Main | % | Main |
| No. | name | (Kabat) | (mg/L) | (° C.) | (° C.) | (° C.) | HMW | Peak | HMW | Peak |
| 3959 | huF11.3 | 2, 27, 37, 45, 47, | 0.7 | NA | NA | NA | NA | NA | NA | NA |
| | | 49, 73, 94 | | | | | | | | |
| 4008 | huF11.10 | 27, 37, 45, 47, 94 | 448.5 | 52.1 | 56.8 | 61.7 | 1.1 | 98.9 | 74.9 | 25.1 |
| 4007 | huF11.11 | 27, 37, 45, 47 | 296.8 | 53.1 | 58.5 | 64.5 | 1.6 | 98.4 | 2.2 | 97.8 |

Thermostability Engineering. VHH 4008 was further engineered to improve thermostability. The amino acid residue at position 24 (according to Kabat) is a buried residue that makes contact to several CDR-supporting residues and was reverted from alanine to asparagine in one variant (SEQ ID NO: 752). The amino acid residue at position73 is a solvent exposed CDR-supporting residue, but was chosen for amino acid substitution from the human valine back to the alpaca serine (i.e., “backmutation”) due to its proximity to CDR3 (SEQ ID NO: 753). The amino acid residue at position 49 is a buried residue that sits at the N-terminus of CDR2 and was substituted from human serine back to alpaca alanine (SEQ ID NO: 754). Finally, the amino acid residue at position 2 is a buried residue and is in close proximity to both CDR1 and CDR3 so was substituted from the human valine to alpaca leucine (SEQ ID NO: 755). All four new variants were tested in a SPR (Carterra) binding assay, DSF melting temperature (Tm) determination, and analytical SEC at baseline and after 50° C. for 1 week. The A24N and V73S amino acid substitutions had no improvement in Tm measurements or aggregation propensity after 50° C. incubation. However, both the V2L and S49A amino acid substitutions had limited aggregation after 50° C. incubation, consistent with an overall improvement in Tm. The results are summarized in Table 16.

(SEQ ID NO: 755)
EVQLLESGGGLVQPGGSLRLSCANSGRTFSSHTVGWFRQAPGKGR
EFVSAITWNGDTTYFGDFVEGRFTISRDNSKNTLYLQMNSLRAED
TAVYYCAAATGQVWSLQYLRESSYAYWGQGTLVTVSS
(SEQ ID NO: 752)
EVQLLESGGGLVQPGGSLRLSCAASGRTFSSHTVGWFRQAPGKGR
EFVSAITWNGDTTYFGDFVEGRFTISRDSSKNTLYLQMNSLRAED
TAVYYCAAATGQVWSLQYLRESSYAYWGQGTLVTVSS
(SEQ ID NO: 753)
EVQLLESGGGLVQPGGSLRLSCAASGRTFSSHTVGWFRQAPGKGR
EFVAAITWNGDTTYFGDFVEGRFTISRDNSKNTLYLQMNSLRAED
TAVYYCAAATGQVWSLQYLRESSYAYWGQGTLVTVSS
(SEQ ID NO: 754)
ELQLLESGGGLVQPGGSLRLSCAASGRTFSSHTVGWFRQAPGKGR
EFVSAITWNGDTTYFGDFVEGRFTISRDNSKNTLYLQMNSLRAED
TAVYYCAAATGQVWSLQYLRESSYAYWGQGTLVTVSS

TABLE 16
Characterization Summary of Humanized VHH
F11 Thermostability Engineering Variants

| | | | | | | | | aSEC, | |
|---|---|---|---|---|---|---|---|---|---|
| | | | | | | aSEC, T0 | | 50° C. 1 week | |
| VHH | Amino Acid | | DSF (Uncle) | | | | % | | % |
| SEQ ID | Substitution | KD | Tonset | TM1 | TM2 | % | Main | % | Main |
| NO: | (Kabat) | (nM) | (° C.) | (° C.) | (° C.) | HMW | Peak | HMW | Peak |
| 752 | A24N | 2.3 | 47.79 | 53.7 | 57 | 0.99 | 99.01 | 100 | 0 |
| 753 | V73S | 1.7 | 50.69 | 57.3 | 62.8 | 1.15 | 98.85 | 58.84 | 41.16 |
| 754 | S49A | 3.6 | 60.27 | 63.5 | 68.1 | 0.77 | 99.23 | 2.08 | 97.72 |
| 755 | V2L | 5.3 | 56.65 | 61.1 | 66.2 | 0.41 | 99.59 | 4.66 | 95.14 |

### Anti-IL-10Rβ VHH (C04) Engineering

Humanization The humanness of alpaca C04 (SEQ ID NO: 461) was assessed using BioPhi (Prihoda, et al., supra). The alpaca VHH was found to contain 72% sequence similarity to human framework IGHV3-23 and human IGHV3-23 was chosen as the acceptor framework for humanization. Homology models of alpaca C04 (SEQ ID NO: 461) and the human IGHV3-23*01 germline sequence were generated using ImmuneBuilder (Abanades, et al., supra) and viewed in PyMol (pymol.org). All residues within 5 Å of Kabat CDRs were defined as “CDR-supporting” residues and were considered for grafting from alpaca framework, in addition to Kabat CDRs, onto the human acceptor framework. All residues that differ between alpaca and human that were greater than 5 Å away from Kabat CDRs were left human. Three humanized variants of alpaca C04 (SEQ ID NO: 461) were generated with increasing similarity to the human germline sequence (Table 17). The generated homology models were analyzed in order to determine which residues CDR-supporting residues that differ between alpaca and human should be grafted onto the acceptor human framework, IGHV3-23*01. The first humanized variant, VHH 4010, was generated by grafting all alpaca Kabat CDRs, CDR supporting residues, and residue 45 (according to Kabat), which has previously been shown to impact VHH CDR-H3 conformation (Bahrami Dizicheh, et al., Commun Biol (2023) 6(1):864), onto the human framework. A more human-like variant, VHH 3964, was generated by substituting residues 49, and 78 of VHH 4010 to the human amino acid. Finally, the third and most human-like variant was generated by grafting only the alpaca Kabat CDRs and residue 45, but not the alpaca CDR-supporting residues, onto the human acceptor framework (SEQ ID NO: 462). Additionally, the glutamine at position 1 was substituted to aspartic acid to reduce the potential of pyro-glutamate formation on all humanized variants. Humanized variant VHH 4010 expressed with >350 mg/L, but the other two humanized variants could not be expressed. VHH 4010 had a Tonset of 56.68° C. and no increase in aggregation upon 50° C. heat stress.

TABLE 17
Characterization Summary of Humanized VHH C04 Variants

| | | | | | | | | | aSEC, | |
|---|---|---|---|---|---|---|---|---|---|---|
| | | | | | | | aSEC, T0 | | 50° C. 1 week | |
| | | Grafted Alpaca | Expression | DSF (Uncle) | | | | % | | % |
| VHH | clone | CDR-Supporting | Titer | Tonset | TM1 | TM2 | % | Main | % | Main |
| No. | name | Residues (Kabat) | (mg/L) | (° C.) | (° C.) | (° C.) | HMW | Peak | HMW | Peak |
| 3963 | huC04.3 | 29, 30, 37, 45, 47 | 3.6 | NA | NA | NA | NA | NA | NA | NA |
| 3964 | huC04.2 | 29, 30, 37, 45, 47, 94 | 0 | NA | NA | NA | NA | NA | NA | NA |
| 4010 | huC04.5 | 29, 30, 37, 45, 47, 49, | 384.9 | 56.68 | 61.79 | 77.05 | 2.7 | 97.3 | 2.45 | 97.55 |
| | | 78, 94 | | | | | | | | |

Humanized F11-C04 Tandem VHH1-VHH2 panel. Tandem VHH1-VHH2-Fc constructs were generated by pairwise combination of each biophysically characterized humanized anti-IL-10Rα or anti-IL-10Rβ VHHs, such that VHH1 was composed of the anti-IL-10Rα binder and VHH2 was composed of the anti-IL-10Rβ binder. The linker between the two VHH domains was a Gly-Gly-Gly-Ser (GGGS; SEQ ID NO: 715), and the linker between the C-terminus of the anti-IL-10Rβ VHHs and the Fc was Gly-Ser (GS). The Fc used for this panel was a silenced human IgG1 isotype, containing the L234A/L235A/G237A amino acid substitutions (Hale, et al., supra). Binding affinity of each humanized tandem VHH1-VHH2 molecule was determined using SPR (Carterra) to either human or cynomolgus IL-10Rα or IL-10Rβ and compared to that of the fully alpaca tandem VHH1-VHH2 parent (SEQ ID NO: 756). Both humanized tandem VHH1-VHH2 molecules maintained within 10-fold affinity difference to the human and cynomolgus IL-10Rα. However, both humanized molecules lost binding to IL-10Rβ. The ability of each humanized tandem VHH1-VHH2 to agonize the IL-10 receptor was tested using a HEK reporter cell line over expressing both IL-10Rα and IL-10Rβ. VHH 3997 (SEQ ID NO: 757) was found to have an about 3.5-fold weaker EC50 than both the alpaca parent molecule and VHH 4013 (SEQ ID NO: 758). VHH 4013 had a higher Emax, however, and was chosen as the humanized tandem VHH1-VHH2 molecule for further evaluation. The results are summarized in Table 18.

TABLE 18
Characterization Summary of Humanized VHH1-VHH2 F11-C04 Tandem VHH1-VHH2 Panel

| | | | | | | IL-10 HEK | |
|---|---|---|---|---|---|---|---|
| | | Binding Affinity (KD, nM) | | | | Reporter Assay | |
| clone | | Human | Cyno | Human | Cyno | EC50 | Emax |
| name | Amino Acid Sequence | IL-10Rα | IL-10Rα | IL-10Rβ | IL-10Rβ | (nM) | (%) |
| Alpaca | SEQ ID NO: 756 | 0.03 | 0.03 | 27 | 116 | 0.32 | 61.3 |
| F11-C04 | ELQLVESGGGLVQAGGSLRLSCANSGRTFSSHTVGWF | | | | | | |
| | RQAPGKEREFVAAITWNGDTTYFGDFVEGRESISRDS | | | | | | |
| | AKNTLYLEMNKLKPEDTAVYYCAAATGQVWSLQYLRE | | | | | | |
| | SSYAYWGQGTQVTVSSGGGSQVQLVESGGGLVQSGGS | | | | | | |
| | LRLSCVASGFTSPYSTIGWERQAPGKEREGVACISSS | | | | | | |
| | GRGVNANYIDSVRGRFTISRDNAKNTVYLQMNKLKPD | | | | | | |
| | DTAIYYCATNCWRLTSPAADNWGQGTQVTVSS | | | | | | |
| huF11.10 | SEQ ID NO: 757 | 0.17 | 0.03 | 117 | NB | 1.03 | 70.5 |
| -huC04.5 | EVQLLESGGGLVQPGGSLRLSCAASGRTFSSHTVGWF | | | | | | |
| | RQAPGKGREFVSAITWNGDTTYFGDFVEGRFTISRDN | | | | | | |
| | SKNTLYLQMNSLRAEDTAVYYCAAATGQVWSLQYLRE | | | | | | |
| | SSYAYWGQGTLVTVSSGGGSEVQLVESGGGLVQPGGS | | | | | | |
| | LRLSCSASGFTSPYSTIGWFRQAPGKGREGVACISSS | | | | | | |
| | GRGVNANYIDSVRGRFTISRDNSKNTVYLQMNSLRAE | | | | | | |
| | DTAVYYCATNCWRLTSPAADNWGQGTMVTVSS | | | | | | |
| huF11.11 | SEQ ID NO: 758 | 0.1 | 0.04 | 138 | NB | 0.23 | 64 |
| -huC04.5 | ELQLLESGGGLVQPGGSLRLSCAASGRTFSSHTVGWF | | | | | | |
| | RQAPGKGREFVAAITWNGDTTYFGDFVEGRFTISRDS | | | | | | |
| | SKNTLYLQMNSLRAEDTAVYYCAAATGQVWSLQYLRE | | | | | | |
| | SSYAYWGQGTLVTVSSGGGSEVQLVESGGGLVQPGGS | | | | | | |
| | LRLSCSASGFTSPYSTIGWFRQAPGKGREGVACISSS | | | | | | |
| | GRGVNANYIDSVRGRFTISRDNSKNTVYLQMNSLRAE | | | | | | |
| | DTAVYYCATNCWRLTSPAADNWGQGTMVTVSS | | | | | | |

huF11.10-huC04.5 (SEQ ID NO: 757) contains a humanized anti-IL-10Rα VHH, huF11.10, shown to have poor thermostability. Therefore, two additional tandem VHH1-VHH2-Fc molecules were generated such that VHH1 was composed of a humanized anti-IL-10Rα VHH containing either the V2L or S49A amino acid substitutions. VHH 184 (SEQ ID NOs: 579, 694) had a higher Emax (Table 19).

TABLE 19

| | | IL-10Rα VHH | | |
|---|---|---|---|---|
| | | Amino Acid | | |
| VHH | | Substitution | IL-10 HEK Reporter Assay | |
| No. | SEQ ID NOS: | (Kabat) | EC50 (nM) | Emax (%) |
| 184 | 579 (Table E) | V2L | 1.09 | 72 |
| | 696 (Table F) | | | |
| 183 | 577 (Table E) | S49A | 1.59 | 52 |
| | 694 (Table F) | | | |

### Example 5

### Structure and Binding Epitope Analysis

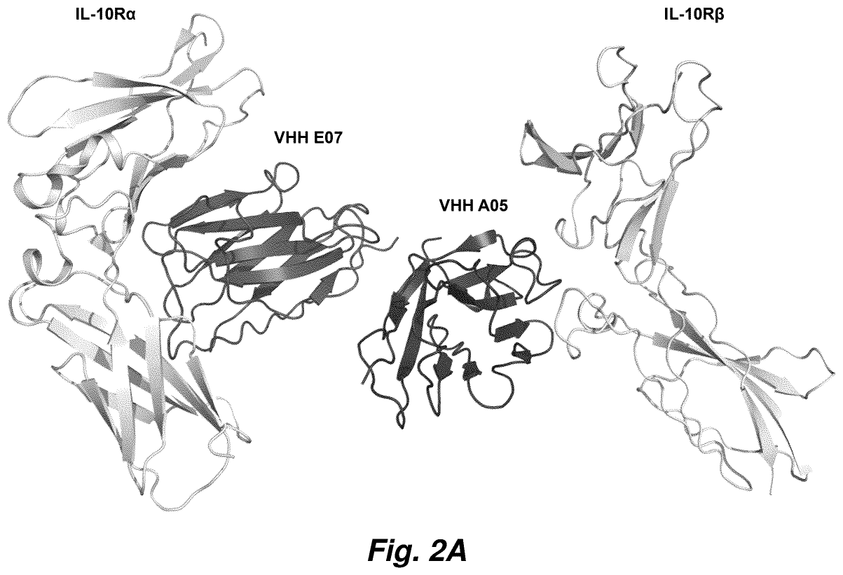

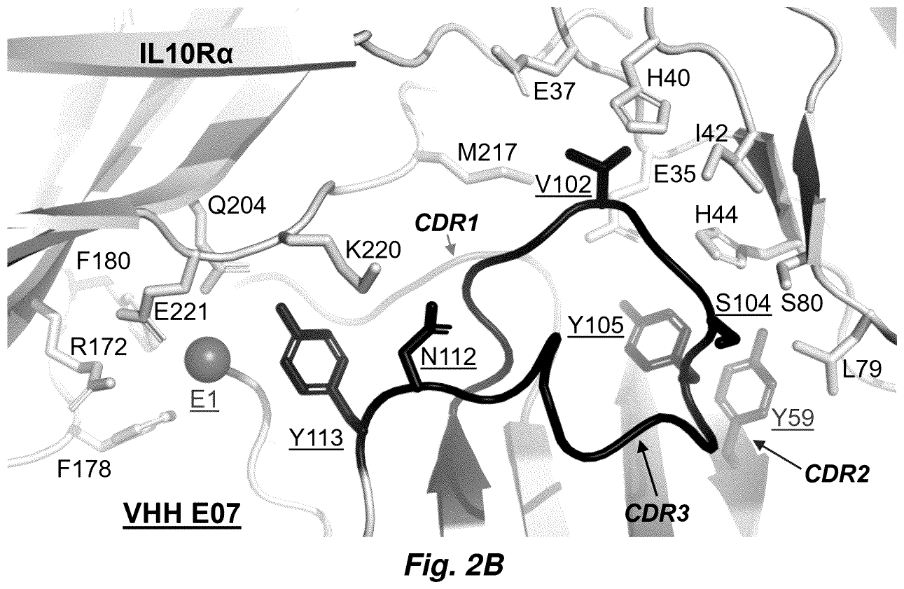

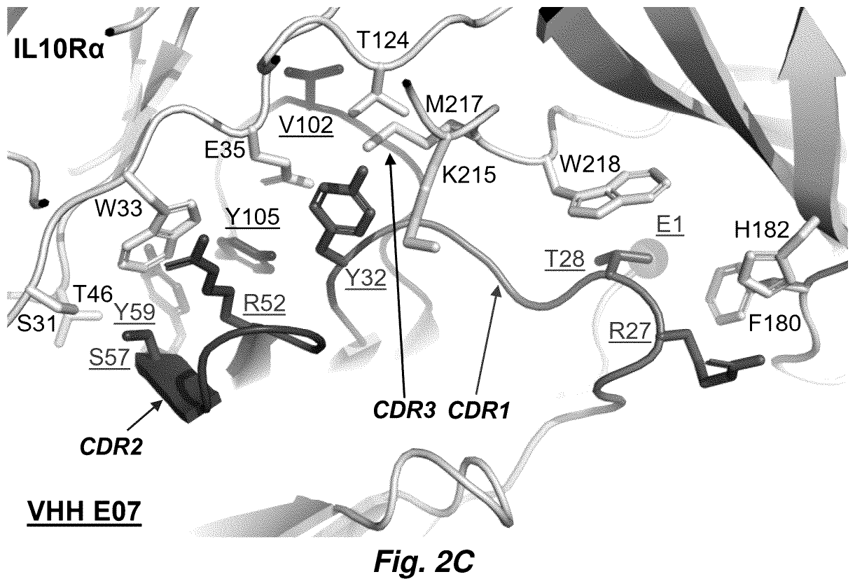

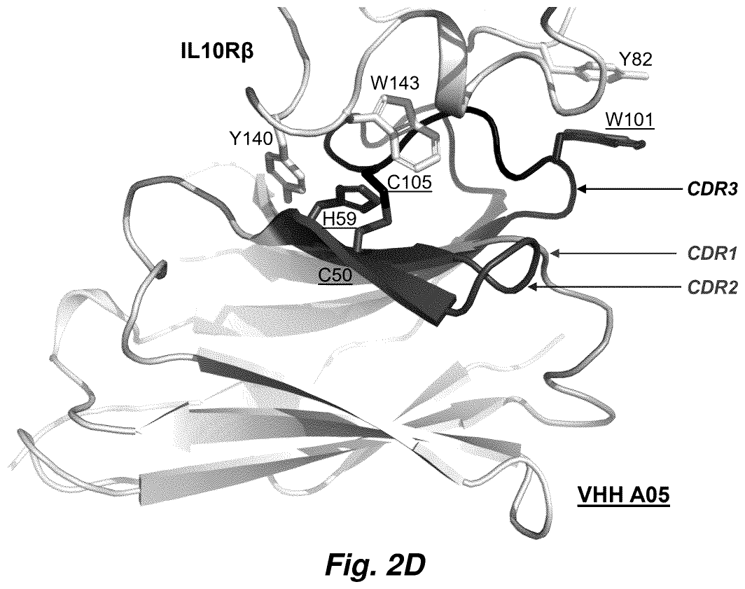

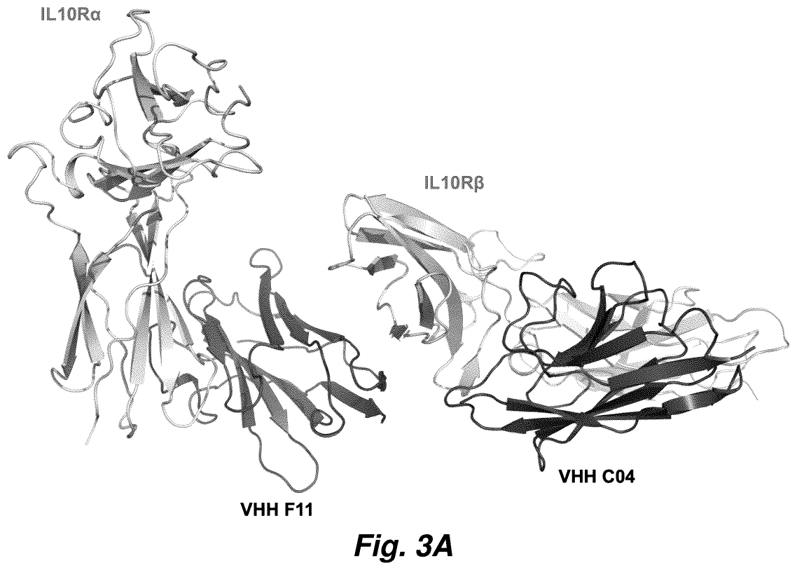

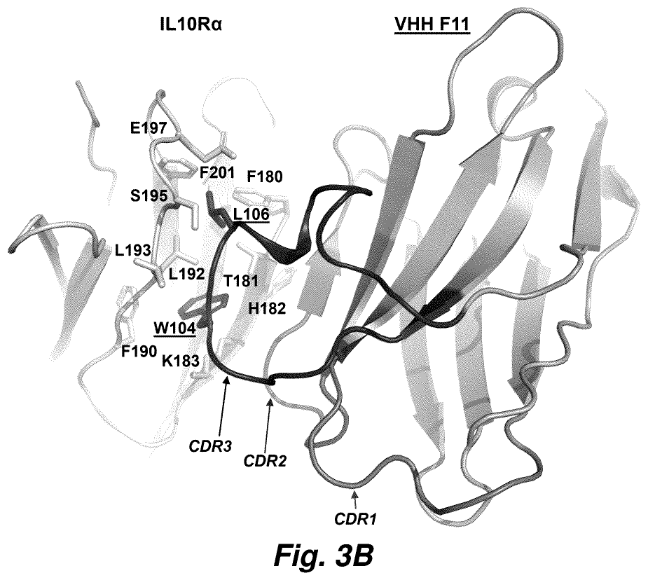

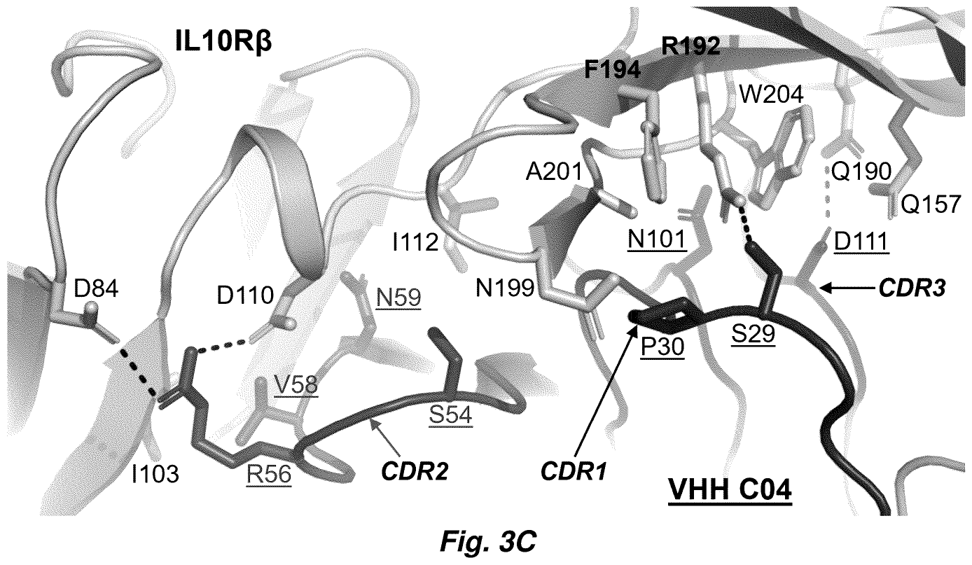

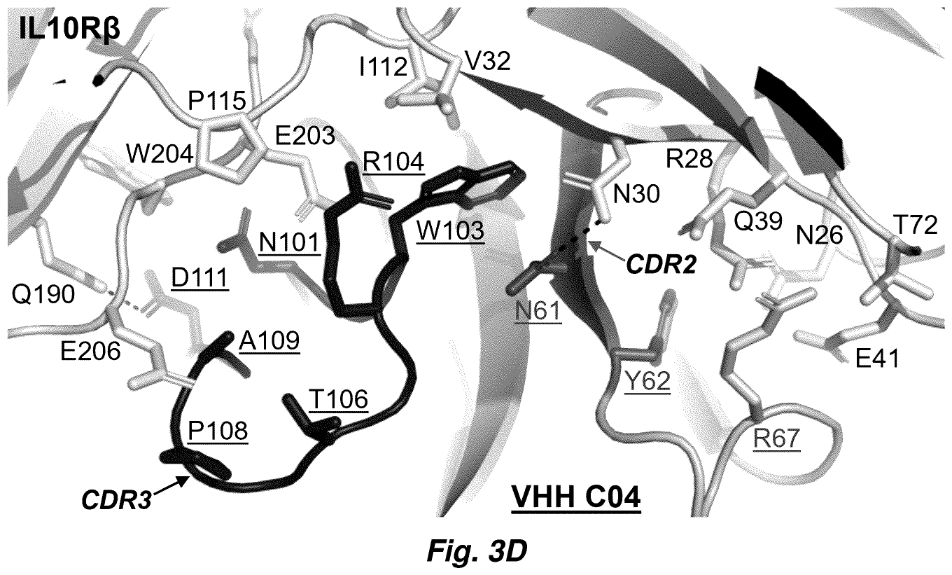

Cryogenic electron microscopy (cryo-EM) was applied to determine structures of IL-10Rα and IL-10Rβ in complexed with tandem VHH1-VHH2 E07-A05 (SEQ ID NO: 759) or F11-C04 (SEQ ID NO: 760) at 3.3 Å and 3.0 Å resolution, respectively. These cryo-EM structures, depicted in FIGS. 2 and 3, show that E07-A05 and F11-C04 have distinct binding sites on IL-10Rα and IL-10Rβ.

Tandem VHH1-VHH2 E07-A05 used for cryo-EM
(SEQ ID NO: 759)
ELQLVESGGGLVQPGGSLRLSCAASGRTFSAYGMGWFRQAPGKGR
EFVAAIRWIGDSTYYADSVKGRFTISKDNSKNTLYLQMNSLRPED
TAVYYCAAKTSVTSYLSEKSYNYWGKGTPVTVSSGGGSEVQLVES
GGGLVQPGGSLRLSCAASGNSLDYPTIGWFRQAPGKGREGVSCIS
ASDGSTHYADSVKGRFTISRDNSKNTLYLQMNSLRPEDTAVYYCA
ASLWGLGCKSEADYDYWGKGTPVTVSSHHHHHH
Tandem VHH1-VHH2 F11-C04 used for cryo-EM
(SEQ ID NO: 760)
ELQLVESGGGLVQAGGSLRLSCANSGRTFSSHTVGWFRQAPGKER
EFVAAITWNGDTTYFGDFVEGRESISRDSAKNTLYLEMNKLKPED
TAVYYCAAATGQVWSLQYLRESSYAYWGKGTPVTVSSGGGSQVQL
VESGGGLVQSGGSLRLSCVASGFTSPYSTIGWFRQAPGKEREGVA
CISSSGRGVNANYIDSVRGRFTISRDNAKNTVYLQMNKLKPDDTA
IYYCATNCWRLTSPAADNWGQGTQVTVSSHHHHHH

### Methods

Cryo-EM sample preparation and data acquisition. To structurally resolve the complexes of IL-10Rα+IL-10Rβ+E07-A05 and IL-10Rα+IL-10Rβ+F11-C04, purified IL-10Rα, IL-10Rβ and tandem VHH1-VHH2 chimera (SEQ ID NO: 759 and 760) were mixed at 1:1:1 molar ratio in Buffer A (25 mM HEPES pH 7.5, 150 mM NaCl) and incubated on ice for 1 hr. An anti-VHH tool Fab has been added to enlarge the size of the complex to facilitate cryo-EM studies. For IL-10Rβ+VHH A05+VHH C04 complex, IL-10Rβ, VHH A05 (SEQ ID NO: 761) and VHH C04 (SEQ ID NO: 762) were mixed at 1:1:1 molar ratio in Buffer A (25 mM HEPES pH 7.5, 150 mM NaCl) and incubated on ice for 1 hr. An anti-VHH tool Fab+anti-Fab tool VHH complex has been added to enlarge the size of the complex to facilitate cryo-EM studies. The complexes were diluted to 1 mg/ml in Buffer A and incubated with 0.05% glutaraldehyde for 5 min on ice in dark. A 3 μL aliquot was applied to a glow-discharged UltrAuFoil grid (R1.2/1.3, Quantifoil). Grids were blotted in Vitrobot Mark IV (Thermo Fisher) for 2 s at 4° C. with 100% humidity, and then plunge-frozen in liquid ethane cooled by liquid nitrogen.

VHH-A05 used for cryo-EM
(SEQ ID NO: 761)
QRQLVESGGGLVQPGGSLRLSCAASGNSLDYPTIGWFRQAPGKER
EGVSCISASDGSTHYADSVKGRFTISRDNTKNTVYLQMNSLEPED
TAVYYCAASLWGLGCKSEADYDYWGKGTPVTVSSHHHHHH
VHH-C04 used for cryo-EM
(SEQ ID NO: 762)
QVQLVESGGGLVQSGGSLRLSCVASGFTSPYSTIGWFRQAPGKER
EGVACISSSGRGVNANYIDSVRGRFTISRDNAKNTVYLQMNKLKP
DDTAIYYCATNCWRLTSPAADNWGQGTQVTVSSHHHHHH

Movies were collected on a Titan Krios (Thermo Fisher) operated at 300 kV using a Falcon 4i direct electron detector (Thermo Fisher) at a nominal magnification of 165,000× (0.729 Å per pixel), using a set defocus range of −0.6 to −1.8 μm with the Selectris X energy filter slit set to 10 eV. Movies were collected in EPU cryo-EM data acquisition software (Thermo Fisher; thermofisher.com/us/en/home/electron-microscopy/products/software-em-3d-vis/epu-software.html) over a 5.85 s exposure with a total dose of ~50 e⁻/Å².

Cryo-EM image processing. The movies were processed using cryoSPARC with Patch Motion Correction followed by estimation of contrast transfer function (CTF) parameters by Patch CTF. Images with CTF fit to 4.5 Å or better were selected. Multiple rounds of 2D classification, ab initio reconstruction followed by non-uniform refinement resulted in the final maps.

Cryo-EM model building. The crystal structures of IL-10Rα, IL-10Rβ and Alphafold models of VHH were used as initial models and docked into the cryo-EM maps by rigid-body fitting. The models were manually adjusted in Crystallographic Object-Oriented Toolkit (Coot) software (www2.mrc-lmb.cam.ac.uk/personal/pemsley/coot) and refined using phenix.real_space_refinement (Adams, et al., Acta Crystallogr D Biol Crystallogr (2010) 66(Pt 2):213-21; phenix-online.org). The Gly-Gly-Gly-Ser (GGGS; SEQ ID NO: 715) linker in-between the tandem VHH1-VHH2 is not resolved in the cryo-EM maps so it is not included in model building.

### Results

The cryo-EM structure of IL-10Rα+IL-10Rβ+E07-A05 shows that CDR1, CDR2 and CDR3 of VHH E07 directly bind to a discontinuous epitope on the antigen target, IL-10Rα (FIGS. 2A and 2B), wherein the discontinuous epitope comprises amino acid residues W33, E35, E37, H40, 142, H44, T46, L79, S80, T124, A170, R172, F178, F180, H182, Q204, K215, M217, W218, K220, and E221 of IL-10Rα when numbered in accordance with SEQ ID NO: 711. The N-term of VHH E07 and T28 in CDR1 binds to a pocket formed by R172, F178, F180, H182, Q204, W218 and E221 of IL-10Rα. Y32 in CDR1, R52, S57 and Y59 in CDR2, and Y105 in CDR3 of VHH E07 interact with W33, E35, H44, T124 and K215 in the inner elbow region of IL-10Rα. V102 in CDR3 of VHH E07 binds to a pocket formed by E35, E37, H40, 142 and H44 of IL-10Rα.

The cryo-EM structure of IL-10Rα+IL-10Rβ+E07-A05 shows that CDR2 and CDR3 of VHH A05 directly bind to a discontinuous epitope on the antigen target, IL-10Rβ (FIGS. 2A and 2C), wherein the discontinuous epitope comprises amino acid residues S78, S80, K81, Y82, V108, N138, E139, Y140, E141, T142, W143, N147, V148, and N150 of IL-10Rβ when numbered in accordance with SEQ ID NO: 712. Loop N138-W143 of IL-10Rβ undergoes a dramatic conformational change upon binding to CDR2 and CDR3 of VHH A05, having the side chains of W143 and Y140 of IL-10Rβ binding to a pocket formed in-between CDR2 and CDR3 of VHH A05 that is stabilized by the disulfide bond formed by C50 in CDR2 and C105 in CDR3. The CDR3 of VHH A05 also interacts with loop S80-Y82 of IL-10Rβ with pi-stacking between side chains of W101 in CDR3 of VHH A05 and Y82 in IL-10Rβ. S54 and D55 in CDR2 of VHH A05 interact with N147 and N150 of IL-10Rβ. Comparing this structure with the published cryo-EM structure of IL-10Rα+IL-10Rβ+IL-10 complex (PDB:6X93), VHH A05 and IL-10 have overlapping binding sites on IL-10Rβ.

The cryo-EM structure of IL-10Rα+IL-10Rβ+F11-C04 shows that CDR2 and CDR3 of VHH-F11 directly bind to a discontinuous epitope on the antigen target, IL-10Rα (FIGS. 3A and 3B), wherein the discontinuous epitope comprises amino acid residues 1169, 1171, K173, F178, F180, T181, H182, K183, K186, F190, L192, L193, 5195, E197, and F201 of IL-10Rα when numbered in accordance with SEQ ID NO: 711. W104 and L106 in CDR3 of VHH-F11 bind to an induced pocket on IL-10Rα formed by F190, L192, L193, S195, E197 and F201 on one side and F180, T181 and K183 on the opposite side. D56, T57 and T58 in CDR2 interact with K186, K183 and H182 of IL-10Rα. F60 and E65 in the frame interact with F180 and F178 of IL-10Rα.

The cryo-EM structure of IL-10Rα+IL-10Rβ+F11-C04 shows that CDR1, CDR2 and CDR3 of VHH C04 directly bind to a discontinuous epitope on the antigen target, IL-10Rβ (FIGS. 3A and 3C), wherein the discontinuous epitope comprises amino acid residues N26, R28, N30, V32, Q39, E41, T72, D84, 1103, T104, D110, 1112, P115, Q157, Q190, R192, F194, N199, A201, E203, W204, and E206 of IL-10Rβ when numbered in accordance with SEQ ID NO: 712.

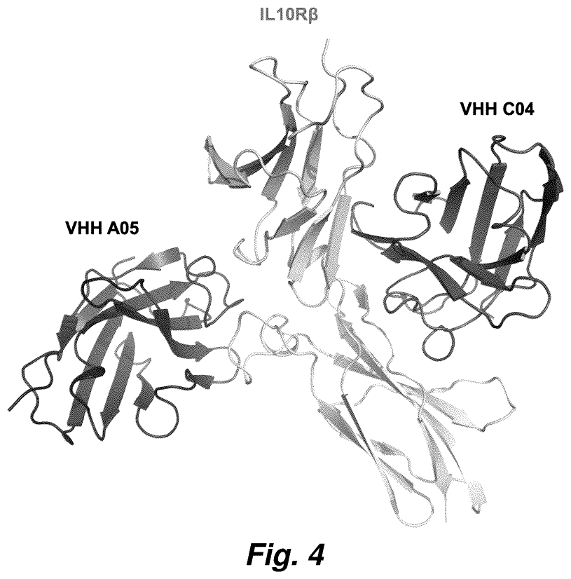

A cryo-EM structure of IL-10Rβ in complexed with VHH A05 (SEQ ID NO: 761) and VHH C04 (SEQ ID NO: 762) was determined at 2.85 Å resolution. This cryo-EM structure, depicted in FIG. 4, shows that VHH A05 and VHH C04 have distinct binding sites on IL-10Rβ and that both VHHs can bind to IL-10Rβ simultaneously.

### Example 6

### Anti-IL-10Rαβ Agonist VHH1-VHH2-Fc Bispecific Molecules 195 and 184 Bind with High Affinity to Both Human IL-10Rα and IL-10Rβ

In this example, we evaluated the biophysical binding kinetics of anti-IL-10Rαβ agonist VHH1-VHH2-Fc bispecific molecules 195 and 184 to recombinant human IL-10Rα and IL-10Rβ.

### Methods

Binding to human IL-10Rα and IL-10Rβ by anti-IL-10Rαβ agonist VHH1-VHH2-Fc bispecific molecules was measured by Surface Plasmon Resonance (SPR) experiments performed on a Biacore 8K instrument. A C1 sensor chip was used to immobilize anti-hu Fc (Southern Biotech, Catalog #2014-01) so bispecific molecules 195 and 184 could be captured via Fc tag. Before use, the C1 chip was preconditioned by injecting two pulses of 0.1M Glycine-NaOH pH 12.0, 0.3% Triton-X100 over the surface for one minute each, followed by injection of dH₂O for one minute each. Surfaces were then activated with sulfo-N-hydroxysuccinimide (s-NHS) and 1-ethyl-3-(3-dimethylaminopropyl)-carbodiimide (EDC). Anti-huFc, diluted in 10 mM sodium acetate pH 5.0 to a final concentration of 10 μg/mL, was then injected over these surfaces. Excess activated surface carboxyls were blocked with an injection of ethanolamine. Final anti-huFc immobilization densities on all channels was about 500 relative units (RU). Bispecific molecule 184 was diluted to 50 nM final concentration in running buffer and captured on each channel, which resulted in captured densities in the range of about 50-60 RU.

For all experiments, the instrument was first stabilized by injecting ten blank buffer injections before injection of analyte samples. A contact time of 180 seconds was used with a dissociation time of 30 minutes. Human and cynomolgus IL-10Rα were injected in a 3-fold concentration series with 150 nM as the highest concentration. Human and cynomolgus IL-10Rβ were injected in a 3-fold concentration series with 1000 nM as the highest concentration. Experiments were performed at 25° C. with a running buffer comprised of 50 mM sodium phosphate pH 7.4, 150 mM NaCl, 0.005% surfactant P20 (Cytiva; cytivalifesciences.com), 10 mM HEPES, pH 7.4, 150 mM NaCl, 1 mM EDTA, 0.005% tween-20, and 0.2 mg/ml BSA. Regeneration between each cycle was performed by injecting a single one-minute pulse of 10 nM Glycine at pH 1.5.

Binding data to human and cynomolgus IL-10Rα and IL-10Rβ were fit either to a simple kinetic model yielding kₒₙ and k_{off} from which K_{D} was calculated (K_{D}=k_{off}/kₒₙ).

The equilibrium dissociation constants (K_{D}) for binding of 195 and 184 to human IL-10Rα (hIL-10Rα) and human IL-10Rβ (hIL-10Rβ) are shown in Table 20.

TABLE 20
Kinetics and Affinities (nM) for VHH1-VHH2-Fc Bispecific Molecules
195 and 184 Binding to Human IL-10-Rα and IL-10-Rβ.

| | | kₐ | kₐ | K_{D} | |
|---|---|---|---|---|---|
| VHH1-VHH2-Fc | Analyte | (1/Ms) | (1/s) | (nM) ± SD | N |
| 195 | hIL-10-Rα | 3.19E+05 | 4.81E−04 | 1.50 ± 0.16 | 3 |
| | hIL-10-Rβ | 3.42E+04 | 5.54E−04 | 16.28 ± 8.83 | 3 |
| 184 | hIL-10-Rα | 8.19E+04 | 1.44E−04 | 1.87 ± 0.56 | 3 |
| | hIL-10-Rβ | 3.10E+05 | 1.79E−02 | 60.10 ± 12.82 | 3 |

### Example 7

### Profiling the Anti-IL-10Rαβ Agonist VHH1-VHH2-Fc Bispecific Molecules in an IL-22/IL-29 Reporter Assay

IL-10Rβ is required by many IL-10 family cytokine signaling including IL-19, IL-22 and IL-29. To determine if the IL-10 receptor agonist VHH1-VHH2-Fc interferes with other IL-10 family cytokine signaling, we profiled five IL-10 receptor agonist VHH1-VHH2-Fc variants in Colo205 IL-22R luciferase reporter assay and HEK293T-IL29R secreted embryonic alkaline phosphatase (SEAP) reporter assay to determine their antagonist activities on other IL-10 family cytokines. VHH1-VHH2-Fc proteins were expressed transiently in Expi293™ cells and purified using protein A and anion exchange chromatography.

### Methods

IL22R pSTAT3 luciferase reporter cell line was developed by overexpressing the human IL-22Rα, human IL-10Rβ, and a Firefly luciferase gene driven by STAT3 response elements in Colo205 cells. IL-29R (IFN-λ) reporter cells was purchased from InvivoGen (Cat #hkb-ifnlv2). One day before the assay, culture medium was used to prepare sample plate with eight points dose-response (1:5 serial dilution). Recombinant human IL-22 (PeproTech, Cat #200-22) or human IL-29 (R&D Cat #1598-IL/CF) was used as positive control. Reporter cells were detached from plate using TrypLE and washed with cell culture media. Cell concentration was adjusted to 8×10⁴ cells/ml and 25 μl (2K cells) of cell suspension was added into each well of solid white tissue culture (TC) 384-well plate (Greiner Cat #781080). On the assay day, five microliters of VHH sample were added to the cell plate and incubated at 37° C. for 1 hour. Then 5 μl of recombinant human IL-22 or IL-29 was added to each well to reach the final concentration of 2 ng/ml. The assay plated then incubate at 37° C. for 3 hours. For IL-22 reporter assay, thirty microliters of Bio-Glo™ Reagent (Promega Cat #G7941) was added into each assay well followed by a brief spin at 1000 rpm. Plate was then incubated at room temperature for 10 mins and read by EnVision plate reader (Revvity). For IL-29 reporter assay, 20 μl of cell supernatant from assay plate was transferred into a separate plate with 10 μl of QUANTI-Blue™ solution (InvivoGen Cat #rep-qbs). The plate then Incubated at room temperature avoiding light for 1 hour followed by absorption quantification at OD 620 nM using EnVision plate reader (Revvity).

The luminescence readout for VHHs treated wells was normalized to average signal from positive (IL-22 or IL-29) and negative (media) controls and expressed as % IL-22R or % IL-29R agonist:

$$\% IL22R / IL29R antagonist = \frac{100 - ( ( Luminescence sample - Luminescence 0 \% activity )}{( Luminescence 100 \% activity - Luminescence 0 \% activity ) ) * 100}$$

### Where

0% IL-22R or % IL-29R antagonist=Signal in wells with IL-22 (2 ng/mL) only
100% IL-22R or % IL-29R antagonist=Signal in wells with media control only

EC₅₀ values were calculated based on log concentration of drug and percent of IL-22 or IL-29 signal using a 4-parameter variable slope nonlinear regression model and least square curve fit (Gilead Science, Foster City, CA).

$$y = \frac{100 \times {EC}_{50}^{m}}{{EC}_{50}^{m} + {[ I ]}^{m}}$$

where y=% Activity, m=Hill coefficient, and [I]=Antagonist concentration.

EC₅₀ values are derived from the average of duplicate data points for each antibody concentration.

Eₘₐₓ is defined as the maximum normalized percentage inhibition of IL22R or IL29R signal achieved in the assay.

### Results

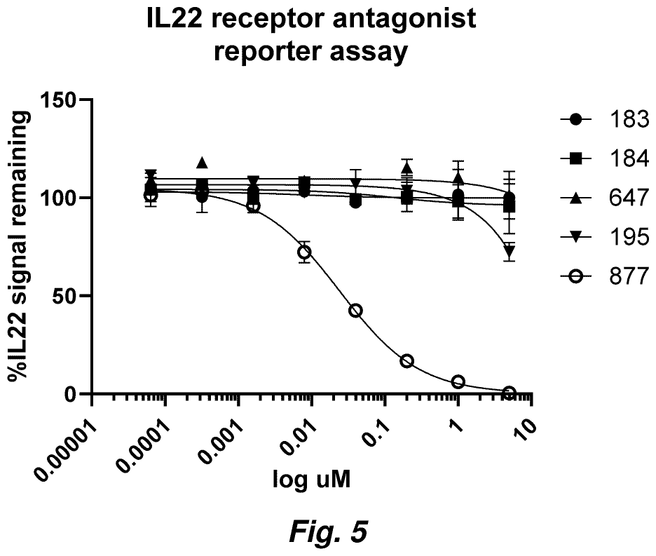

A total of five human VHH1-VHH2 bispecific molecules were expressed, purified, and profiled in Colo205 IL-22R-pSTAT3 luciferase reporter assay. Recombinant human IL-22 induced a robust receptor activation at 2 ng/ml (60 pM about EC₉₀). Molecule 877 (SEQ ID NO: 595) potently blocked IL-22 signaling with 24.8 nM EC₅₀ and 100% of inhibition Eₘₐₓ, while the other four VHH1-VHH2-Fc demonstrated minimal or no inhibition of IL-22 signaling at the highest tested concentration. The results are shown in FIG. 5.

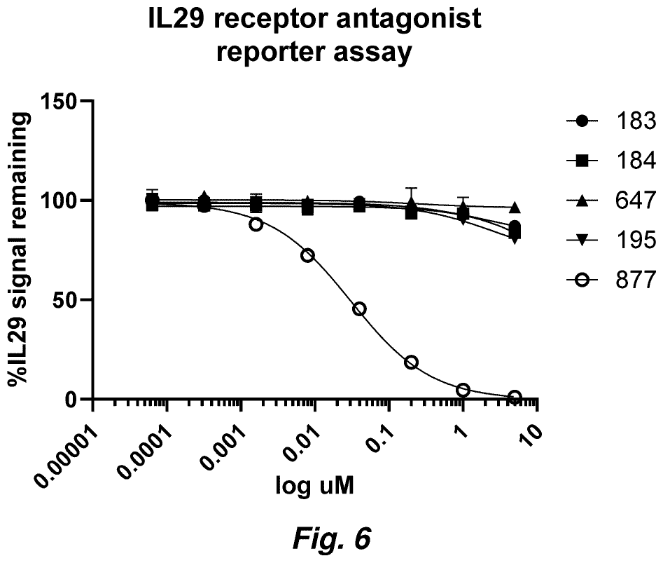

A total of five human VHH1-VHH2 bispecific molecules were expressed, purified, and profiled in HEK293T IL-29 reporter assay. Recombinant human IL-29 induced a robust receptor activation at 2 ng/ml (100 pM about EC₈₅). Molecule 877 (SEQ ID NO: 595) potently blocked IL-29 signaling with 30.1 nM EC₅₀ and 100% of inhibition Eₘₐₓ, while the other four VHH1-VHH2-Fc demonstrated minimal or no inhibition of IL-29 signaling at the highest tested concentration. The results are shown in FIG. 6.

### Example 8

### Profiling the Anti-IL-10Rαβ VHH1-VHH2-Fc Molecules in Human Peripheral Blood Mononuclear Cells

In this example, IL-10Rα and IL-10Rβ is expressed by human immune cells of monocytes, B cells, T cells, and NK cells. We tested the selectivity of IL-10R agonism in different cell types in human blood.

### Methods

Human whole blood was drawn and equilibrated to room temperature and 90 μL was pipetted into 96-Deep-Well plates and prewarmed to 37° C. for at least 15 minutes prior to addition of molecules. Ten (10) μL of the 10× molecule dose titration stock was added and incubated for an additional 15 minutes at 37° C. At the end of the incubation, RBCs were lysed and fixed by adding 900 μL of BD Lyse/Fix Buffer (BD, Cat No. 558049) prepared via manufacturer's instructions, mixed well 8-10 times, and incubated 10 minutes at 37° C. Cells were pelleted and washed twice with PBS+0.5% BSA and then stained with the following antibodies for 20 minutes at room temperature: CD3 BUV805 (BD Cat No. 612895), CD8 BUV737 (BD Cat No. 612754), CD4 BUV396 (BD Cat No. 563550), CD19 BV786 (BD Cat No. 563325), CD56 BV711 (BD Cat No. 563169), CD14 BV421 (BD Cat No. 563743).

Cells were then washed twice with PBS+0.5% BSA and permeabilized by adding 1 mL cold methanol and incubated for 20 minutes at 4° C. Cells were washed once with PBS, twice with PBS+0.5% BSA and then stained with the following antibodies for 1 hour at 4° C.: Stat1 (pY701) PE (BD Cat No. 562069), Stat3 (pY705) Alexa Fluor 647 (BD Cat No. 562071), CD15 PerCP-Cy5.5 (Biolegend Cat No. 323020).

Cells were pelleted and washed once with PBS+0.5% BSA and resuspend to read on BD Fortessa. Samples were run on a BD Fortessa and gated on relevant cell populations after first gating on forward scatter area (FSC-A) by forward scatter height forward scatter height (FSC-H) to exclude aggregates and doublets. Statistics and Inhibition IC50 Values: mean fluorescence intensity (MFI) for each cell population, donor and compound treatment was calculated from the signal detected phosphorylated STAT.

### Results

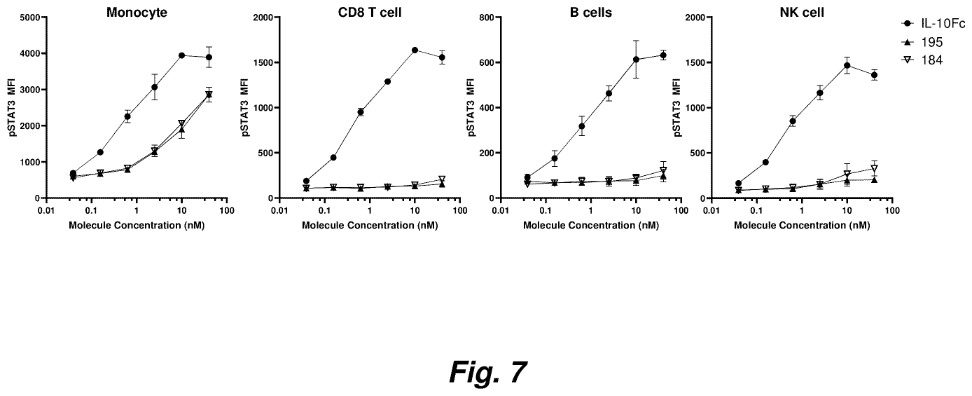

Anti-IL-10Rαβ VHH1-VHH2-Fc bispecific molecules 184 and 195 (SEQ ID NOs: 696 and 599, respectively) induced STAT3 phosphorylation in human monocytes but not in T cells, B cells, or NK cells. IL-10-Fc was included as a positive control that induced STAT3 phosphorylation in monocyte, T cells, B cells, and NK cells. These results demonstrate the selectivity of VHH1-VHH2 bispecific molecules 184 and 195 in monocytes and myeloid cells. The results are shown in FIG. 7.

### Example 9

### Anti-IL-10Rαβ VHH1-VHH2-Fc Molecules Potently Inhibited the LPS-Induced IL-6 Secretion from Human Peripheral Blood Mononuclear Cells (PBMCs)

In this example, we profiled anti-IL-10Rαβ VHH1-VHH2-Fc bispecific molecules and IL-10 regulate proinflammatory cytokine release by LPS stimulation.

### Methods

IL-10 receptor agonist activity of anti-IL-10Rαβ VHH1-VHH2-Fc bispecific molecules 184 and 195 were evaluated in human PBMCs upon LPS stimulation. In this assay, anti-IL-10Rαβ VHH1-VHH2-Fc were pre-diluted as 20-point dose responses in media. Frozen PBMCs from 3 different donors were thawed, allowed to recover for about 2 hours, and dispensed into 384-well plates at 5000 cells/well in 40 μL media. Five microliter of sample dilutions were added to the cells, mixed, and pre-incubated at 37° C. for 30 minutes. Five microliters of 10×LPS solution (10 ng/mL final concentration) was added to pre-treated cells and plates were incubated at 37° C. overnight for about 20-22 hours. Forty microliters per well of supernatant was collected and 7 μL of that was used for IL-6 quantification using LANCE Ultra TR-FRET Detection Kit (Revvity Part number: TRF1223C). Recombinant IL-10 was used as positive control and cells in media with and without LPS stimulation were used as negative controls. Data were normalized and % control=100%*(well−pos)/(neg−pos). EC₅₀ values were calculated based on log concentration of drug and normalized percent of IL-10 signal using a 4-parameter variable slope nonlinear regression model and least square curve fit.

### Results

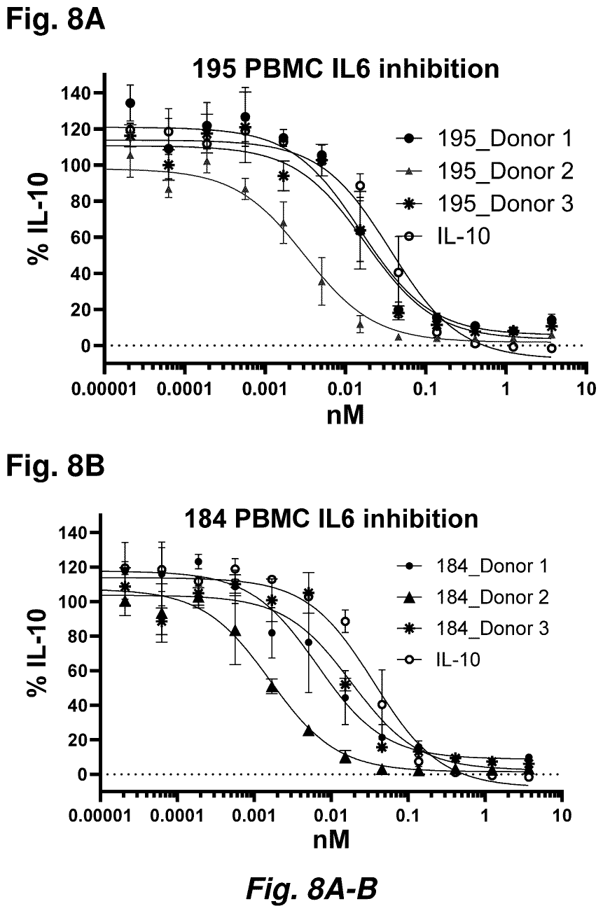

Recombinant wild-type human IL-10 induced a robust IL-10 receptor activation on PBMC, which potently inhibit LPS-induced IL-6 secretion with 36 pM. Whereas anti-IL-10Rαβ VHH1-VHH2-Fc bispecific molecules 195 and 184 also achieved 92 and 95 percent cytokine inhibition, respectively, in comparison to wild-type IL-10, with EC₅₀ of 10.17 pM and 6.18 pM for IL-6 secretion from 3 donors. The results are summarized in Table 21 and depicted in FIG. 8. The results demonstrate that anti-IL-10Rαβ VHH1-VHH2-Fc bispecific molecules 195 and 184 are potent IL-10 receptor agonists (single digit pM) in primary PBMC, which was able to fully block LPS induced IL-6 secretion from PBMC.

TABLE 21
Anti-IL-10Rαβ VHH1-VHH2-Fc Bispecific Molecules
Inhibition of Primary PBMC IL-6 Secretion in vitro

| | wt IL-10 | 195 | 184 |
|---|---|---|---|
| Avg. EC₅₀ (pM) | 36.27 ± 20 | 10.17 ± 6 | 6.18 ± 5 |
| Avg Eₘₐₓ % | 100 | 93 ± 2 | 95 ± 2 |

### Example 10

### Anti-IL-10Rαβ VHH1-VHH2-Fc Bispecific Molecules Induce Minimal IFN-γ Secretion from Primary CD8+ T Cells

In this example, IL-10 receptor agonist activity of anti-IL-10Rαβ VHH1-VHH2-Fc bispecific molecules 195 and 184 on primary human T cells were evaluated by measuring IFN-γ secretion from human CD8+ T-cells upon stimulation.

### Methods

In this assay, sample of anti-IL-10Rαβ VHH1-VHH2-Fc bispecific molecules 195 and 184 were pre-diluted as 8-point dose responses in media. Frozen human CD8+ T-cells were thawed, allowed to recover and expand for about 3 days with anti-CD3 and anti-CD28 antibodies, then dispensed into 384-well plates at 5000 cells/well in 40 μL media. Five microliter of antibody dilutions were added to the cells, mixed, and pre-incubated at 37° C. for 3 additional days. Five microliters of anti-CD3 and anti-CD28 (5 μg/mL each) was added to pre-treated cells and plates were incubated at 37° C. for about 6 hours. Thirty-five microliters per well of supernatant was collected and 10 μL of that was used for IFN-γ quantification using Meso Scale Discovery (MSD) U-PLEX human IFN-γ (Catalog number K151TTK). Recombinant wild-type IL-10 was used as positive control and cells in media with and without IL-10 stimulation were used as negative controls. Data were normalized and % control=100%*(well−neg)/(pos−neg). EC₅₀ values were calculated based on log concentration of drug and normalized percent of IL-10 signal using a 4-parameter variable slope nonlinear regression model and least square curve fit.

### Results

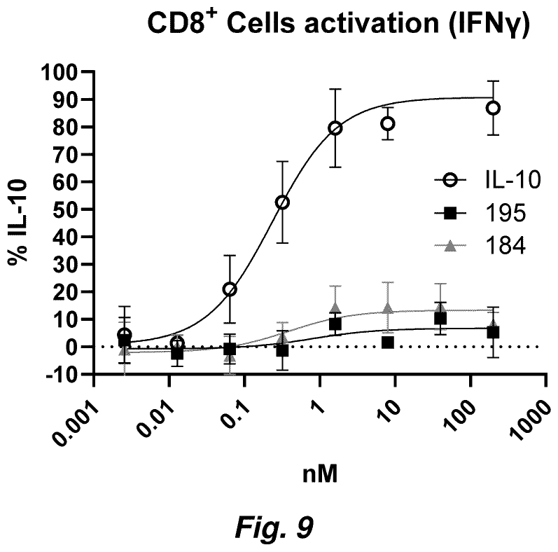

Recombinant human IL-10 induced a robust IL-10 receptor activation on CD8+ T cells, which induced significant IFN-γ secretion with an EC₅₀ of 28 pM. Anti-IL-10Rα VHH1-VHH2-Fc bispecific molecules 195 and 184 induced 10% and 12% percent of IFN-γ secretion with no detectable EC₅₀, respectively, in comparison to IFN-γ secretion induced by wild-type IL-10. The results are depicted in FIG. 9. These results demonstrate that anti-IL-10Rαβ VHH1-VHH2-Fc bispecific molecules 195 and 184 does not significantly active primary CD8+ T cells.

### Example 11

### Comparator Anti-IL-10Rαβ VHH1-VHH2-Fc Bispecific Molecule Inhibits Monocyte Cytokine Release, but Also Promotes STAT3 Phosphorylation, IFN-γ, and Granzyme B Release in T Cells

We generated an anti-IL-10Rαβ VHH1-VHH2-Fc bispecific comparator molecule (C1), based on IL-10Rα and IL-10Rβ VHH antigen binding domains disclosed in WO 2022/032022 (SEQ ID NOs: 170 and 197 in the '022 publication), in a VHH1-VHH2-Fc format, as described herein. The amino acid sequence of anti-IL-10Rαβ VHH1-VHH2-Fc bispecific comparator molecule C1 is as follows:

(SEQ ID NO: 788)
QVQLQESGGGSVQAGGSLRLSCAASGYSNCSYDMTWYRQAPGKER
EFVSAIHSDGSTRYADSVKGRFFISQDNAKNTVYLQMNSLKPEDT
AMYYCKTDPLHCRAHGGSWYSVRANYWGQGTQVTVSSGGGSQVQL
QESGGGSVQAGGSLRLSCAASGYSYSSYCMGWFRQAPGKEREGVA
TIDSDGMTRYADSVKGRFTISKDNAKNTLYLQMNSLKPEDTAMYY
CAAPLYDCDSGAVGRNPPYWGQGTQVTVSSASRVESKYGPPCPPC
PAPEFLGGPSVFLFPPKPKDTLMISRTPEVTCVVVDVSQEDPEVQ
FNWYVDGVEVHNAKTKPREEQFGSTYRVVSVLIVLHQDWLNGKEY
KCKVSNKGLPSSIEKTISKAKGQPREPQVYTLPPSQEEMTKNQVS
LTCLVKGFYPSDIAVEWESNGQPENNYKTTPPVLDSDGSFFLYSR
LTVDKSRWQEGNVFSCSVMHEALHNHYTQKSLSLSLG

### Methods

IL-10R agonist activity in monocytes was evaluated in human PBMCs upon LPS stimulation. In this assay, anti-IL-10Rαβ VHH1-VHH2-Fc bispecific comparator molecule C1 was pre-diluted in media. Frozen PBMCs from two different donors were thawed, allowed to recover for about 2 hours, and dispensed into 96-well plates. Ten (10) ng/mL LPS was added to pre-treated cells and plates were incubated at 37° C. overnight for about 20-22 hours. TNFα was quantified by V-PLEX human TNF-α KIT (Meso Scale Discovery). Recombinant wild-type IL-10 was used as positive control and cells in media with and without LPS stimulation were used as negative controls.

For phosphorylated STAT3 quantification in whole blood immune cells, human blood was drawn and equilibrated to room temperature and 90 μL was pipetted into 96-Deep-Well plates and prewarmed to 37° C. for at least 15 minutes prior to addition of molecules. Ten (10) μL of the 10× molecule dose titration stock was added and incubated for an additional 15 minutes at 37° C. At the end of the incubation, RBCs were lysed and fixed by adding 900 μL of BD Lyse/Fix Buffer (BD, Cat No. 558049) prepared via manufacturer's instructions, mixed well 8-10 times, and incubated 10 minutes at 37° C. Cells were pelleted and washed twice with PBS+0.5% BSA and then stained with the following antibodies for 20 minutes at room temperature: CD3 BUV805 (BD Cat No. 612895), CD8 BUV737 (BD Cat No. 612754), CD4 BUV396 (BD Cat No. 563550), CD19 BV786 (BD Cat No. 563325), CD56 BV711 (BD Cat No. 563169), CD14 BV421 (BD Cat No. 563743).

Then cells were washed twice with PBS+0.5% BSA and permeabilized by adding 1 mL cold methanol and incubated for 20 minutes at 4° C. Cells were washed once with PBS, twice with PBS+0.5% BSA and then stained with the following antibodies for 1 hour at 4° C.: Stat1 (pY701) PE (BD Cat No. 562069), Stat3 (pY705) Alexa Fluor 647 (BD Cat No. 562071), CD15 PerCP-Cy5.5 (Biolegend Cat No. 323020).

Cells were pelleted and washed once with PBS+0.5% BSA and resuspended to read on BD Fortessa. Samples were run on a BD Fortessa and gated on relevant cell populations after first gating on FSC-A by FSC-H to exclude aggregates and doublets. Statistics and Inhibition IC50 Values: MFI for each cell population, donor and compound treatment will be calculated from the signal detected phosphorylated STAT.

For T cell stimulation assay, IL-10 or comparison molecule were pre-diluted as 10-point dose responses in media. Frozen human T-cells were thawed, allowed to recover and expand for about 3 days with anti-CD3 and anti-CD28 antibodies, then dispensed into 384-well plates at 5000 cells/well in 40 μL media. Five microliter of antibody dilutions were added to the cells, mixed, and pre-incubated at 37° C. for 3 additional days. Five microliters of anti-CD3 and anti-CD28 (5 μg/mL each) was added to pre-treated cells and plates were incubated at 37° C. for about 6 hours. Thirty-five (35) microliter per well of supernatant was collected and 10 μL of that was used for IFN-γ quantification using MSD U-PLEX human IFN-γ and granzyme-B (Catalog number K151TTK). Recombinant IL-10 was used as positive control and cells in media with and without IL-10 stimulation were used as negative controls. Data were normalized and % control=100%*(Well−Neg)/(Pos−Neg). EC50 values were calculated based on log concentration of drug and normalized percent of IL-10 signal using a 4-parameter variable slope nonlinear regression model and least square curve fit.

### Results

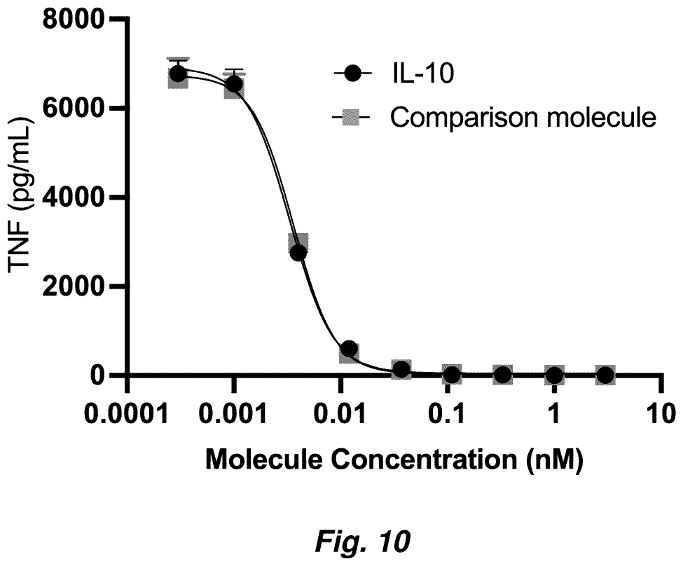

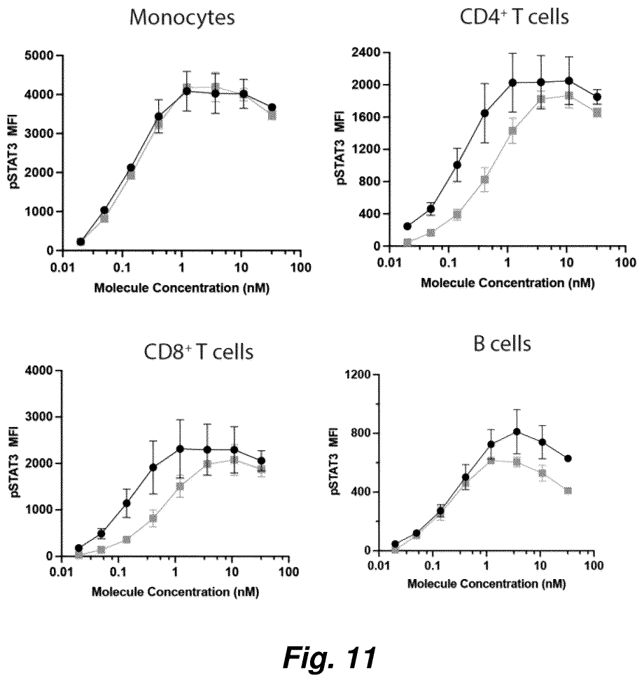

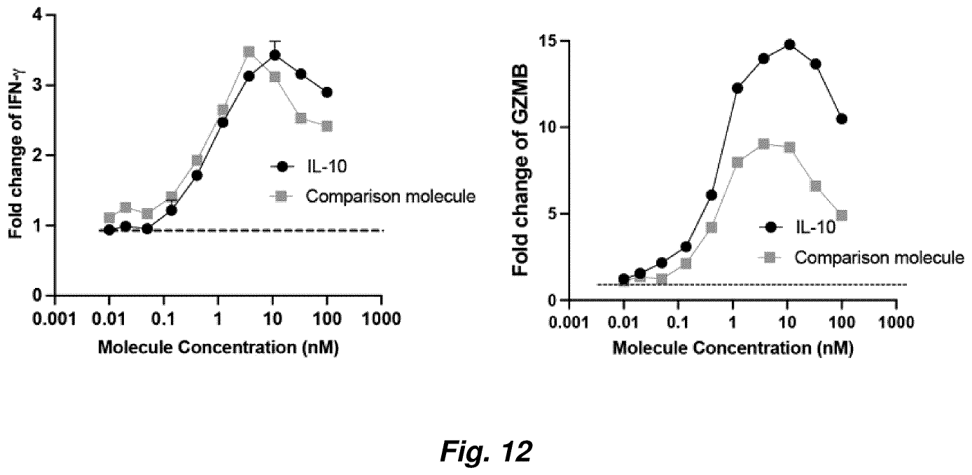

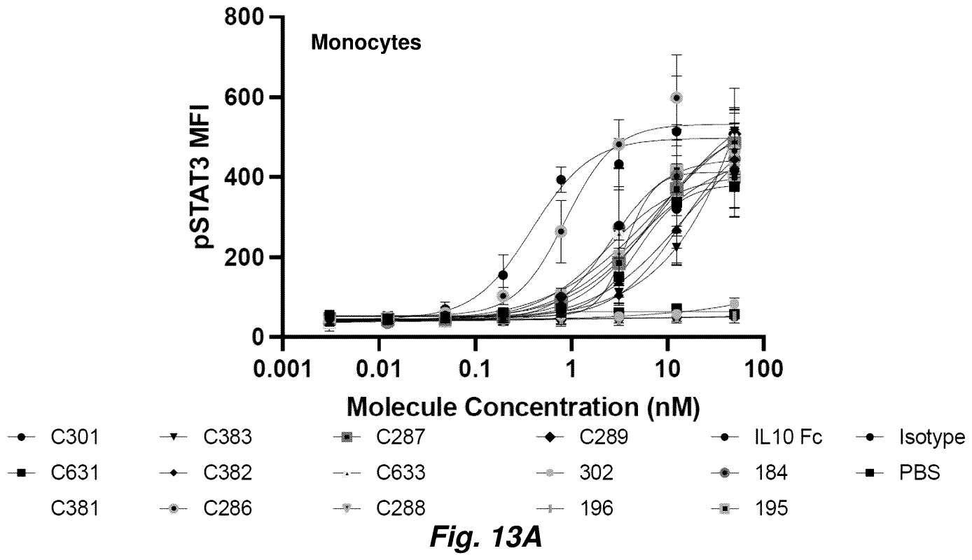

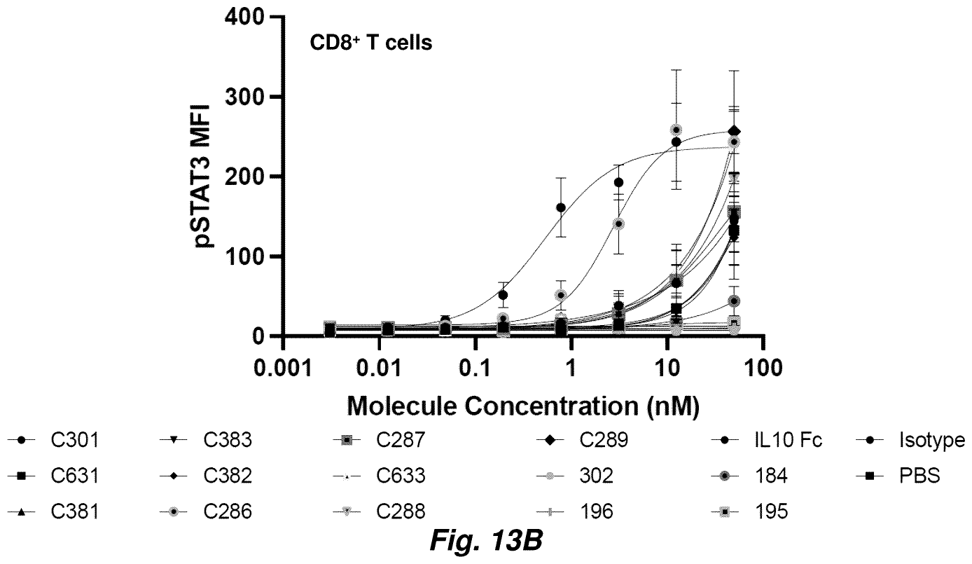

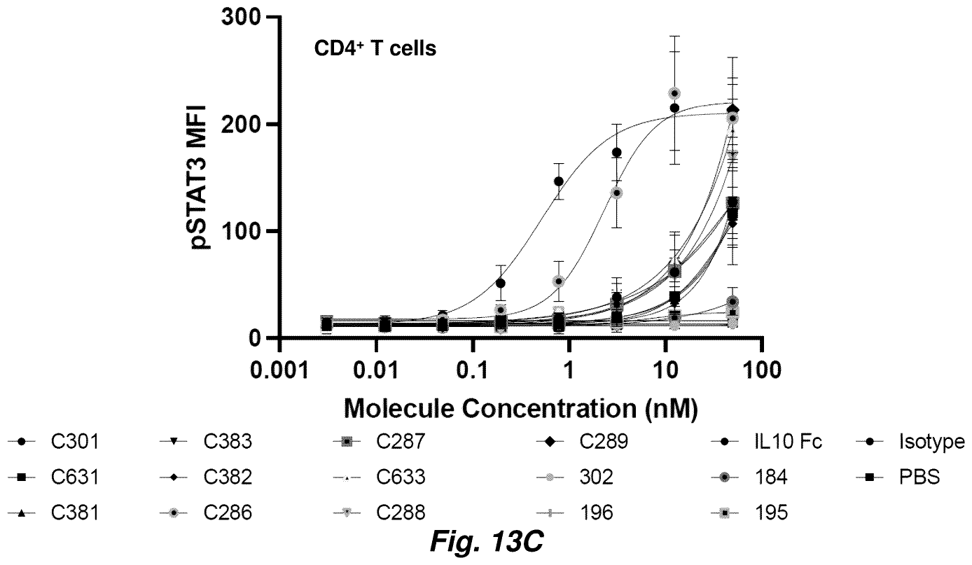

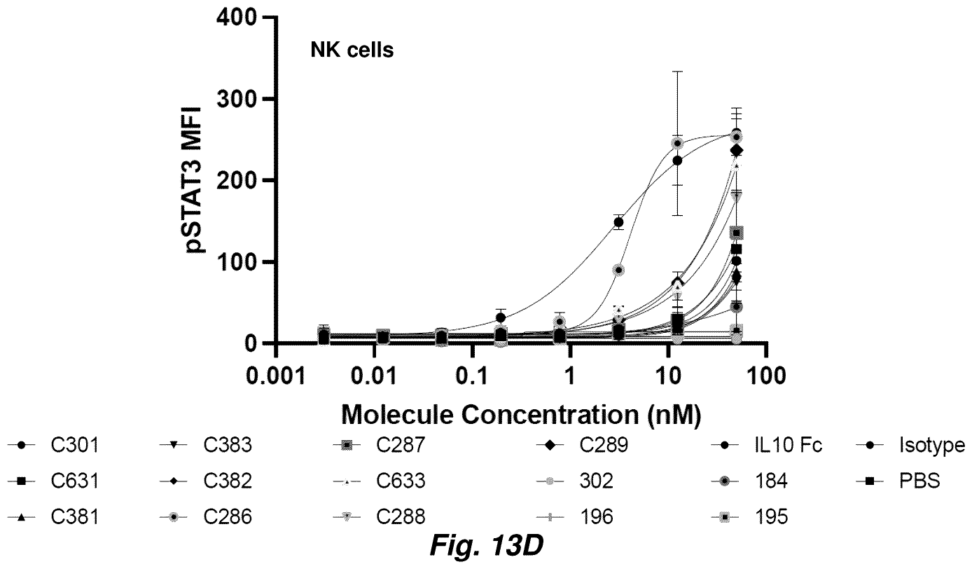

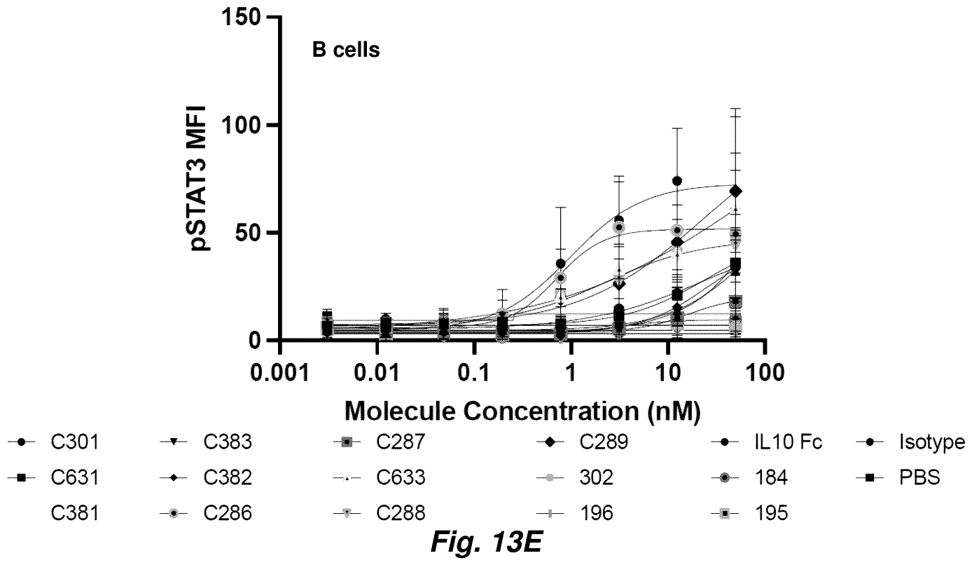

The anti-IL-10Rαβ VHH1-VHH2 bispecific comparator molecule C1 showed similar biological activity as compared with wild-type IL-10. C1 inhibited LPS-induced TNFα release in human PBMC comparable to IL-10 (see, FIG. 10). Unlike the anti-IL-10Rα VHH1-VHH2 bispecific molecules described herein, C1 also triggered STAT3 phosphorylation in CD4+ T cells, CD8+ T cells, and B cells, however (see, FIG. 11). Additionally, unlike the anti-IL-10Rαβ VHH1-VHH2 bispecific molecules described herein, comparator molecule C1 promoted activated T cells to secrete IFN-γ and granzyme-B (see, FIG. 12).

### Example 12

### Analysis of IL-10Rαβ VHH1-VHH2-Fc and Comparator Molecules in Inhibiting LPS-Induced IL-6 Secretion from Human Peripheral Blood Mononuclear Cells (PBMCs)

In this example, IL-10RA and IL-10RB were expressed by human immune cells of monocytes, B cells, T cells, and NK cells. We tested the selectivity of IL-10R agonism in different cell types in human blood.

We generated anti-IL-10Rαβ VHH1-VHH2 bispecific comparator molecules (C301, C381, C382, C383 and C631), based on IL-10Rα and IL-10Rβ VHH antigen binding domains disclosed in WO 2024/254567, in a VHH1-VHH2-Fc format, as described herein. The amino acid sequences of anti-IL-10Rαβ VHH1-VHH2 bispecific comparator molecules C301, C381, C382, C383 and C631, respectively, are as follows:

(C301; SEQ ID NO: 789)
EVQLQESGGGSVQAGGSLRLSCAASGYSNCAYDMTWYRQAPGKER
EFVSAIHSDGSTRYADSVKGRFFISQDNAKNTVYLQMNSLKPEDT
AMYYCKTDPLHCRAHGGSWYSVRANYWGQGTQVTVSSGGGSQVQL
QESGGGSVEAGGSLRLSCAASGYTHSSYCMGWFRQAPGKEREGVA
AIDVDGSTTYADSVKGRFTISKDNAKNTLYLQMNSLKPEDTGMYY
CAAEFADCSSNYFLPPGAVRYWGQGTQVTVSS
(C381; SEQ ID NO: 790)
EVQLLESGGGLVQAGGSLRLSCAASGYSNCAYDMTWYRQAPGKER
EFVSAIHSDGSTRYADSVKGRFFISRDNSKNTVYLQMNSLRPEDT
AVYYCKTDPLHCRAHGGSWYSVRANYWGQGTLVTVSSGGGSEVQL
VESGGGLVQPGGSLRLSCAASGYTHSSYCMGWVRQAPGKGLEGVS
AIDVDGSTTYADSVKGRFTISRDNSKNTLYLQMNSLRAEDTAVYY
CAAEFADCSSNYFLPPGAVRYWGQGTTVTVSS
(C382; SEQ ID NO: 791)
EVQLLESGGGLVQAGGSLRLSCAASGYSNCAYDMTWYRQAPGKER
EFVSAIHSSGSTYYADSVKGRFFISRDNSKNTVYLQMNSLRPEDT
AVYYCKTDPLHCRAHGGSWYSVRANYWGQGTLVTVSSGGGSEVQL
VESGGGLVQPGGSLRLSCAASGYTHSSYCMGWVRQAPGKGLEGVS
AIDVDGSTTYADSVKGRFTISRDNSKNTLYLQMNSLRAEDTAVYY
CAAEFADCSSNYFLPPGAVRYWGQGTTVTVSS
(C383; SEQ ID NO: 792)
EVQLLESGGGLVQAGGSLRLSCAASGYSNCAYDMTWYRQAPGKER
EFVSAIHSDGSTYYADSVKGRFFISRDNSKNTVYLQMNSLRPEDT
AVYYCKTDPLHCRAHGGSWYSVRANYWGQGTLVTVSSGGGSEVQL
VESGGGLVQPGGSLRLSCAASGYTHSSYCMGWVRQAPGKGLEGVS
AIDVDGSTTYADSVKGRFTISRDNSKNTLYLQMNSLRAEDTAVYY
CAAEFADCSSNYFLPPGAVRYWGQGTTVTVSS
(C631; SEQ ID NO: 793)
EVQLLESGGGLVQAGGSLRLSCAASGYSNCAYDMTWYRQAPGKER
EFVSAIHSDGSTRYADSVKGRFFISRDNSKNTVYLQMNSLRPEDT
AVYYCKTDPLHCRAHGGSWYSVRANYWGQGTLVTVSSGGGSEVQL
VESGGGLVQPGGSLRLSCAASGYTHSSYCMGWVRQAPGKGLEGVS
AIDVDGSTTYADSVKGRFTISRDNSKNTLYLQMNSLRAEDTAVYY
CAAEFADCSSNYFLPPGAVRYWGQGTLVTVSS
Anti-IL-10Rαβ VHH1-VHH2 bispecific comparator
molecules C301, C381, C382, C383 and C631
were evaluated with and without
an Fc (IgG1 with 234A, 235A and 237A).
(SEQ ID NO: 794)
GSDKTHTCPPCPAPEAAGAPSVELFPPKPKDTLMISRTPEVTCVV
VDVSHEDPEVKENWYVDGVEVHNAKTKPREEQYNSTYRVVSVLTV
LHQDWLNGKEYKCKVSNKALPAPIEKTISKAKGQPREPQVYTLPP
SREEMTKNQVSLTCLVKGFYPSDIAVEWESNGQPENNYKTTPPVL
DSDGSFFLYSKLTVDKSRWQQGNVFSCSVMHEALHNHYTQKSLSL
SPGK

### Methods

Human whole blood was drawn and equilibrated to room temperature and 90 μL was pipetted into 96-Deep-Well plates and prewarmed to 37° C. for at least 15 minutes prior to addition of molecules. Ten (10) μL of the 10× molecule dose titration stock was added and incubated for an additional 15 minutes at 37° C. At the end of the incubation, RBCs were lysed and fixed by adding 1800 μL of BD Lyse/Fix Buffer (BD, Cat No. 558049) prepared via manufacturer's instructions, mixed well 8-10 times, and incubated 15 minutes at 37° C. Cells were pelleted and washed twice with PBS+0.5% BSA and then stained with the following antibodies for 20 minutes at room temperature: CD3 BUV805 (BD Cat No. 612895), CD8 BUV737 (BD Cat No. 612754), CD4 BUV396 (BD Cat No. 563550), CD19 BV786 (BD Cat No. 563325), CD56 BV711 (Biolegend Cat No. 318336), CD14 BV421 (BD Cat No. 563743). Then cells were washed twice with PBS+0.5% BSA and permeabilized by adding 1 mL cold methanol and incubated for 20 minutes at 4° C. Cells were washed once with PBS, twice with PBS+0.5% BSA and then stained with the following antibodies for 1 hour at 4° C.: Stat1 (pY701) PE (BD Cat No. 562069), Stat3 (pY705) Alexa Fluor 647 (BD Cat No. 562071)

Cells were pelleted and washed once with PBS+0.5% BSA and resuspended to read on BD Fortessa. Samples were run on a BD Fortessa and gated on relevant cell populations after first gating on FSC-A by FSC-H to exclude aggregates and doublets. Statistics and Inhibition IC50 Values: mean fluorescence intensity (MFI) for each cell population, donor and compound treatment were calculated from the signal detected phosphorylated STAT.

### Results

Anti-IL-10Rαβ VHH1-VHH2 bispecific molecules 184 and 195 selectively induced STAT3 phosphorylation in human monocytes and significantly less in T cells, B cells, or NK cells. IL-10-Fc, included as a positive control, induced STAT3 phosphorylation in monocytes, T cells, B cells, and NK cells, serving as a benchmark. The results are summarized in Table 22.

Table 22 summarizes the myeloid selectivity of anti-IL-10Rαβ VHH1-VHH2 bispecific molecules 195 and 184 compared to comparator molecules C301, C381, C382, C383 and C631. Data are presented as maximal pSTAT3 phosphorylation relative to the IL-10-Fc molecule. The ratio of maximal pSTAT3 induction in blood monocytes versus CD8+ T cells serves as a parameter to compare myeloid cell selectivity relative to T cells.

These results highlight the selectivity of anti-IL-10Rαβ VHH1-VHH2 bispecific molecules 184 and 195 for monocytes and myeloid cells compared to comparator molecules. Notably, the VHH1-VHH2-Fc bispecific versions of C301, C381, C382, C383 and C631 exhibited more than 3-fold enhanced STAT3 phosphorylation in CD8+ T cells as compared with molecules 184 and 195.

TABLE 22

| | | | | | | | | Ratio of |
|---|---|---|---|---|---|---|---|---|
| | | Monocytes | Emax % IL-10-Fc | | | | | pSTAT3 |
| Antibody | | pSTAT3 | | CD8+ | CD4+ | | | (Monocytes vs |
| ID | Format | EC50 nM | Monocytes | T cells | T cells | NK cells | B cells | T cells) |
| 195 | VHH-VHH-Fc bivalent | 3.71 | 77.8 | 6.97 | 11.22 | 14.5 | 10.4 | 11.2 |
| 302 | VHH-VHH-His monovalent of 195 | >1000 | 16.26 | 4.77 | 6.27 | 10.92 | 12.16 | 3.4 |
| 184 | VHH-VHH-Fc bivalent | 2.64 | 86.76 | 18.04 | 15.86 | 42.33 | 13.53 | 4.81 |
| 196 | VHH-VHH-His monovalent of 184 | >1000 | 10.1 | 4.32 | 5.98 | 13.28 | 7.27 | 2.33 |
| C301 | VHH-VHH-His monovalent | 2.44 | 81.31 | 60.64 | 59.08 | 95.2 | 45.73 | 1.34 |
| C286 | VHH-VHH-Fc bivalent version of C301 | 0.87 | 116.43 | 106.16 | 106.32 | 238.23 | 70.71 | 1.10 |
| C381 | VHH-VHH-His monovalent | 14.82 | 83.18 | 54.36 | 53.09 | 82.82 | 43.68 | 1.53 |
| C633 | VHH-VHH-Fc bivalent version of C381 | 8.1 | 97.45 | 98.4 | 90.61 | 206.69 | 83.15 | 0.99 |
| C382 | VHH-VHH-His monovalent | 13.82 | 86.37 | 50.68 | 49.67 | 75.71 | 44.71 | 1.7 |
| C289 | VHH-VHH-Fc bivalent version of C382 | 9.3 | 98.81 | 105.38 | 98.98 | 223.16 | 93.62 | 0.93 |
| C383 | VHH-VHH-His monovalent | 410.1 | 100.16 | 56.66 | 59.59 | 70.76 | 48.89 | 1.76 |
| C288 | VHH-VHH-Fc bivalent version of C383 | 4.99 | 81.87 | 81.55 | 79 | 168.55 | 59.8 | 1.0 |
| C631 | VHH-VHH-His monovalent | 4.93 | 73.4 | 54.44 | 54.14 | 108.76 | 48.53 | 1.35 |
| C287 | VHH-VHH-Fc bivalent version of C631 | 6.66 | 14.29 | 64.05 | 58.7 | 127.45 | 24.62 | 0.22 |
| 558 | Isotype | >1000 | 9.8 | 6.07 | 6.45 | 15.73 | 8.31 | 1.61 |

### Example 13

### Comparative Analysis of Anti-IL-10Rαβ VHH1-VHH2-Fc and Comparator Molecules in Inhibiting LPS-Induced IL-6 Secretion from Human Peripheral Blood Mononuclear Cells

In this example, we profiled anti-IL-10Rαβ VHH1-VHH2 bispecific molecules 184 and 195, and anti-IL-10Rαβ VHH1-VHH2 bispecific comparator molecules C301, C381, C382, C383 and C631 in PBMC IL-6 release assay post-LPS stimulation.

### Methods

The IL-10 receptor agonist activity of anti-IL-10Rαβ VHH1-VHH2 bispecific molecules 184 and 195, and anti-IL-10Rαβ VHH1-VHH2 bispecific comparator molecules (C301, C381, C382, C383 and C631) were evaluated in human peripheral blood mononuclear cells (PBMCs) following LPS stimulation. Anti-IL-10Rαβ VHH1-VHH2-Fc constructs were pre-diluted in media to generate 8-point dose-response curves. Cryopreserved PBMCs from two distinct donors were thawed, allowed to recover for approximately 2 hours, and dispensed into 384-well plates at a density of 5,000 cells per well in 40 μL of media. Subsequently, 5 μL of each sample dilution was added to the wells, mixed gently, and pre-incubated at 37° C. for 30 minutes. LPS was then added by dispensing 5 μL of a 10× stock solution to achieve a final concentration of 10 ng/mL. Plates were incubated overnight at 37° C. for approximately 20-22 hours. Following incubation, 40 μL of supernatant was collected from each well, and 7 μL was used for IL-6 quantification using the LANCE® Ultra TR-FRET Detection Kit (Revvity, Part Number: TRF1223C). Recombinant IL-10 served as a positive control, while media-only and LPS-only conditions were included as negative controls. Data were normalized using the formula: % Control=100×(Well−Positive)/(Negative−Positive) EC₅₀ values were calculated based on the log-transformed drug concentrations and normalized IL-10 signal percentages using a four-parameter variable slope nonlinear regression model with least squares curve fitting.

### Results

All tested samples demonstrated potent inhibition of LPS-induced IL-6 secretion from PBMCs. The IC₅₀ values ranged from 5.4 to 313.6 pM, with maximum inhibition levels between 86% and 99% relative to IL-10 (Table 23). Notably, the Fc-fused versions of the molecules exhibited greater potency, as indicated by lower IC₅₀ values, compared to their tandem His-tagged counterparts.

TABLE 23
Comparative Potency Analysis of Anti-IL-10Rαβ VHH1-VHH2-Fc and Comparator Molecules in a Primary PBMC IL-6 Secretion Assay

| | | | | | IL-6 Emax | IL-6 Emax | PBMC IL-6 Emax |
|---|---|---|---|---|---|---|---|
| Antibody | | IL-6 IC50 (pM) | IL-6 IC50 (pM) | IL-6 IC50 (pM) | % IL-10 | % IL-10 | % IL-10 |
| ID | Format | Donor 1 | Donor 2 | Averages ± SD | Donor 1 | Donor 2 | Averages ± SD |
| 195 | VHH-VHH-Fc bivalent | 24.0 | 15.2 | 19.6 ± 6.2 | 94 | 95 | 94.3 ± 0.4 |
| 184 | VHH-VHH-Fc bivalent | 33.5 | 23.0 | 28.3 ± 7.5 | 96 | 95 | 95.8 ± 0.7 |
| C301 | VHH-VHH-His monovalent | 102.7 | 49.3 | 76.0 ± 37.7 | 94 | 92 | 93.1 ± 1.3 |
| C286 | VHH-VHH-Fc bivalent version of C301 | 8.7 | 10.3 | 9.5 ± 1.1 | 98 | 98 | 98.2 ± 0.1 |
| C381 | VHH-VHH-His monovalent | 364.4 | 313.6 | 339.0 ± 35.9 | 93 | 86 | 89.5 ± 4.9 |
| C633 | VHH-VHH-Fc bivalent version of C381 | 30.9 | 29.3 | 30.1 ± 1.2 | 97 | 99 | 97.9 ± 0.9 |
| C382 | VHH-VHH-His monovalent | 295.1 | 253.3 | 274.2 ± 29.6 | 93 | 92 | 92.4 ± 0.7 |
| C289 | VHH-VHH-Fc bivalent version of C382 | N/A | 5.4 | 5.4 | | 98 | 98.0 |
| C383 | VHH-VHH-His monovalent | 235.1 | 270.0 | 252.6 ± 24.7 | 92 | 90 | 90.9 ± 1.5 |
| C288 | VHH-VHH-Fc bivalent version of C383 | N/A | 5.6 | 5.6 | N/A | 98 | 98.0 |
| C631 | VHH-VHH-His monovalent | 218.5 | 124.1 | 171.3 ± 66.7 | 93 | 90 | 91.5 ± 1.6 |
| C287 | VHH-VHH-Fc bivalent version of C631 | 127.1 | 84.1 | 105.6 ± 30.4 | 95 | 94 | 94.5 ± 0.2 |

### Example 14

### Comparative Analysis of Anti-IL-10Rαβ VHH1-VHH2-Fc and Comparator Molecules in IFN-γ Secretion from Primary CD8+ T Cells

In this study, the IL-10 receptor agonist activity of anti-IL-10Rαβ VHH1-VHH2 bispecific molecules 184 and 195, and anti-IL-10Rαβ VHH1-VHH2 bispecific comparator molecules C301, C381, C382, C383 and C631 were evaluated by measuring IFN-γ secretion from primary human CD8+ T cells following stimulation.

### Methods

The IL-10 receptor agonist activity of anti-IL-10Rαβ VHH1-VHH2 bispecific molecules 184 and 195, and anti-IL-10Rαβ VHH1-VHH2 bispecific comparator molecules C301, C381, C382, C383 and C631 was assessed by measuring IFN-γ secretion from primary human CD8+ T cells from two distinct donors following stimulation. Test molecules were pre-diluted in media to generate 8-point dose-response curves. Cryopreserved human CD8+ T cells were thawed, allowed to recover and expand for approximately 3 days in the presence of anti-CD3 and anti-CD28 antibodies, and then dispensed into 384-well plates at a density of 5,000 cells per well in 40 μL of media. Subsequently, 5 μL of each antibody dilution was added to the wells, mixed gently, and pre-incubated at 37° C. for an additional 3 days. Following pretreatment, 5 μL of anti-CD3 and anti-CD28 antibodies (final concentration: 5 μg/mL each) were added to each well, and plates were incubated at 37° C. for approximately 6 hours. Supernatants (35 μL per well) were collected, and 10 μL was used for IFN-γ quantification using the MSD U-PLEX Human IFN-γ Assay (Catalog No. K151TTK). Recombinant IL-10 was included as a positive control, while media-only and IL-10-free conditions served as negative controls. Data were normalized using the formula: % Control=100×(Well−Negative)/(Positive−Negative) EC₅₀ values were calculated based on the log-transformed drug concentrations and normalized IL-10 signal percentages using a four-parameter variable slope nonlinear regression model with least squares curve fitting.

### Results

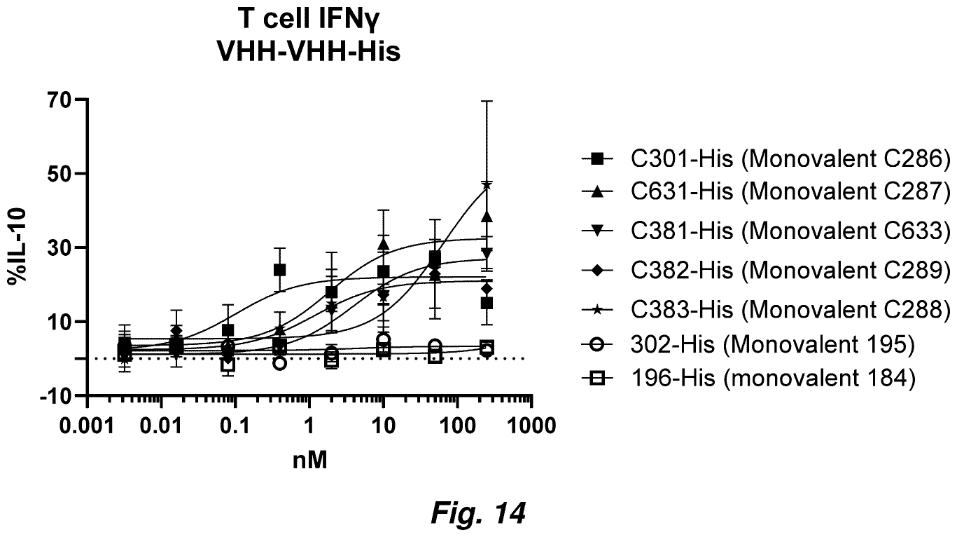

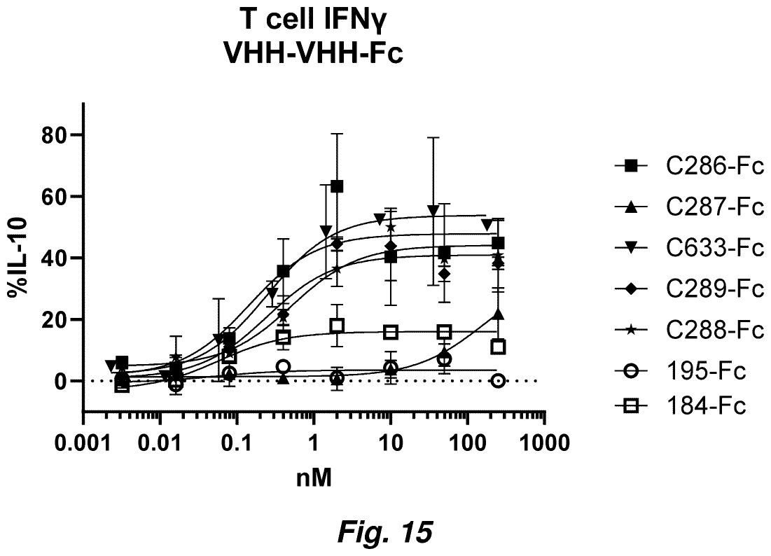

Recombinant human IL-10 induced robust activation of the IL-10 receptor and subsequent IFN-γ secretion from activated CD8⁺ T cells, serving as the benchmark for data normalization. Monovalent VHH1-VHH2-His variants 302 (monovalent form of 195) and 196 (monovalent form of 184) did not elicit detectable IFN-γ secretion from activated CD8⁺ T cells. In contrast, comparator molecules in the same format induced significantly higher IFN-γ secretion, with Emax values ranging from 25% to 38.5% relative to recombinant human IL-10. For bivalent VHH1-VHH2-Fc format molecules, 195 and 184 induced only 2.5% and 15.5% IFN-γ secretion, respectively, while comparator molecules in the same format showed markedly higher activity, with Emax values ranging from 15% to 58.5% of the recombinant IL-10 benchmark. These results, summarized in Table 24 and depicted in FIGS. 14-15, are consistent with the conclusion that molecules 195 and 184 exhibit greater selectivity compared to comparator molecules, with minimal agonist activity on primary CD8⁺ T cells.

TABLE 24
Comparative Potency Analysis of Anti-IL-10Rαβ
VHH1-VHH2-Fc and Comparator Molecules in a Primary T
cell IFN-γ Secretion Assay

| | | IFN-γ | IFN-γ | IFN-γ |
|---|---|---|---|---|
| | | Emax % | Emax % | Emax % |
| Antibody | | IL-10 | IL-10 | IL-10 |
| ID | Format | Donor 1 | Donor 2 | Averages ± SD |
| 302 | VHH-VHH-His | 0 | 0 | 0 |
| | monovalent | | | |
| | of 195 | | | |
| 195 | VHH-VHH-Fc | 5 | 0 | 2.5 ± 3.5 |
| | bivalent | | | |
| 196 | VHH-VHH-His | 0 | 0 | 0 |
| | monovalent | | | |
| | of 184 | | | |
| 184 | VHH-VHH-Fc | 17 | 14 | 15.5 ± 2.1 |
| | bivalent | | | |
| C301 | VHH-VHH-His | 25 | 25 | 25.0 ± 0 |
| | monovalent | | | |
| C286 | VHH-VHH-Fc | 47 | 48 | 47.5 ± 0.7 |
| | bivalent version | | | |
| | of C301 | | | |
| C381 | VHH-VHH-His | 31 | 19 | 25.0 ± 8.5 |
| | monovalent | | | |
| C633 | VHH-VHH-Fc | 53 | 60 | 56.5 ± 4.9 |
| | bivalent version | | | |
| | of C381 | | | |
| C382 | VHH-VHH-His | 20 | 25 | 22.5 ± 3.5 |
| | monovalent | | | |
| C289 | VHH-VHH-Fc | 41 | 69 | 55.0 ± 19.8 |
| | bivalent version | | | |
| | of C382 | | | |
| C383 | VHH-VHH-His | 45 | 32 | 38.5 ± 9.3 |
| | monovalent | | | |
| C288 | VHH-VHH-Fc | 43 | 74 | 58.5 ± 21.9 |
| | bivalent version | | | |
| | of C383 | | | |
| C631 | VHH-VHH-His | 32 | 26 | 29.0 ± 4.2 |
| | monovalent | | | |
| C287 | VHH-VHH-Fc | 15 | 15 | 15.0 ± 0 |
| | bivalent version | | | |
| | of C631 | | | |

### Example 15

### Anti-IL-10Rαβ VHH1-VHH2-Fc Molecules Inhibit Dendritic Cell Differentiation, Activation, and Cytokine Production

In addition to monocytes, dendritic cells (DC) are a key myeloid cell population that plays a role in promoting IL-10 dependent immune tolerance. We evaluated how anti-IL-10αβ bispecific VHH1-VHH2-Fc bispecific molecules affect dendritic cell differentiation, activation, and cytokine production.

### Methods

For DC differentiation, CD14+ monocytes were isolated using EasySep™ Human CD14 Positive Selection Kit II (Stemcell Cat No. 1000694). Purified monocytes were washed and isolated monocytes were resuspended at 5×10⁶ cells/ml and plated in a 96-well tissue culture dish at 100 μl/well. For differentiation, cells were incubated in complete RPMI media with human IL-4 (Peprotech) at 40 ng/ml and GMCSF (Peprotech) at 40 ng/ml for 6 days. Fresh media containing IL-4 and GMCSF were added on day 3. To test the inhibition potency, both IL-10-Fc and VHH1-VHH2 bispecific molecules 195 and 184 (SEQ ID NOs: 696 and 599, respectively) were added at 10 nM during the whole differentiation process. After differentiation, DCs were tested for levels of expression of CD1a PE/Dazzle™ 594 (Biolegend Cat No. 300132) and CD14 BV421 (BD Cat No. 563743).

For DC activation/maturation, CD14+ monocytes were first differentiated to DCs as described above. Successfully differentiated DCs were stimulated with zymosan (100 ng/ml) alone or with 10 nM IL-10 Fc and VHH1-VHH2-Fc bispecific molecules 195 or 184 for 24 hours. After maturation, DCs were tested for levels of expression of CD83 APC-Cy7 (Biolegend Cat No. 305330) and CD86 BV605 (Biolegend Cat No. 374213). Cell supernatants were isolated and levels of TNFα, and IL-23 were measured by MSD assays as per the manufacturer's instructions.

### Results

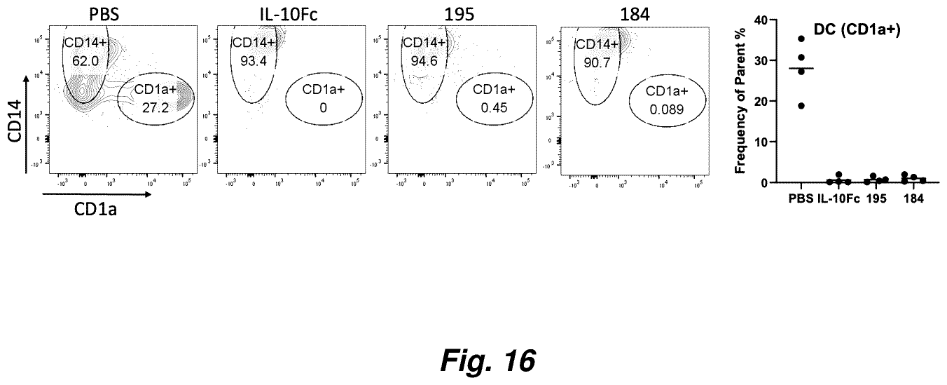

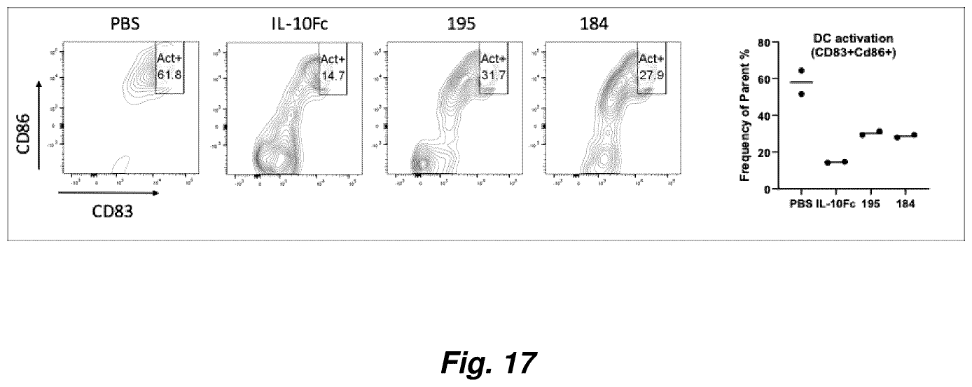

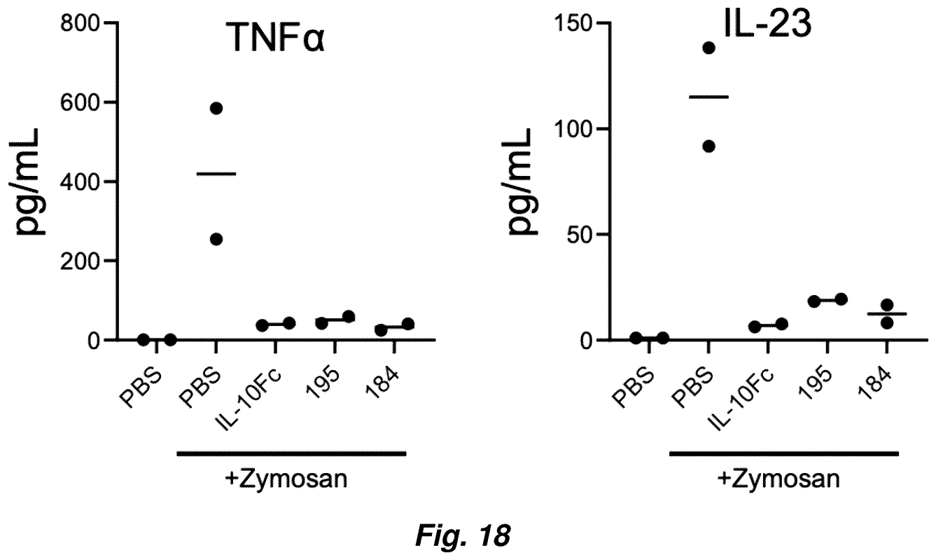

The results are shown in FIGS. 16-18. FIG. 16 shows that VHH1-VHH2 bispecific molecules 195 and 184 inhibited monocyte differentiation into dendritic cells (defined as CD1a+CD14− cells). FIG. 17 shows that VHH1-VHH2 bispecific molecules 195 and 184 inhibited LPS-induced dendritic cell maturation, as measured by the expression of DC activation marker CD83 and co-stimulatory marker CD86. FIG. 18 shows that VHH1-VHH2 bispecific molecules 195 and 184 inhibited zymosan-induced dendritic cell secretion of TNFα and IL-23 The data are consistent with the conclusion that anti-IL-10Rαβ VHH1-VHH2 bispecific molecules 195 or 184 are highly potent and effective in suppress DC differentiate, DC activation, and inhibit DC production of proinflammatory cytokine TNFα and IL-23 in responding to zymosan treatment.

### Example 16

### Anti-IL-10Rαβ VHH1-VHH2-Fc Molecules Inhibit Antigen Specific T-Cell Expansion

IL-10 is an immunosuppressive cytokine that regulates immune responses by inhibiting the ability of antigen presenting cells (APCs) to present antigens to T cells and reducing antigen specific T-cell expansion. In this example, we co-cultured dendritic cells with an influenza antigen hemagglutinin peptide (HA307) antigen specific T-cell and quantified antigen specific T-cell expansion.

### Methods

For dendritic cell (DC) preparation, CD14+ monocytes were isolated using human CD14 microBeads (Miltenyi Biotec, 130-050-201). Isolated monocytes were then differentiated and matured using human monocyte-derived dendritic cell differentiation kit (R & D systems, CDK004). CD14+ monocytes were resuspended at 10⁶ cells/ml and plated in a 24-well tissue culture dish at 1 ml/well. To test the inhibition potency, isotype control, IL-10-Fc and VHH1-VHH2 bispecific molecules 195 or 184 were added at 1 nM or 10 nM during the whole process of differentiation and maturation whenever changing the media. On day 7, in addition to huIL-4, GM-CSF and TNFα and IL-10 molecules, 10 ng/ml LPS was also added for DC maturation, and 0.1 ug/ml influenza antigen Hemagglutinin peptide (HA307) (Influenza A HA 307-319, DRA1*01:01/DRB1*04:01, PKYVKQNTLKLAT (SEQ ID NO: 763)) was added for peptide pulsing. On day 9, the dendritic cells were washed and resuspended at 10⁶ cells/ml.

For T cell preparation, DRB1*04:01 donor PBMCs were obtained from BioIVT. Influenza HA307 reactive CD4+ T cells were stimulated and expanded by 0.1 μg/ml HA307 peptide on day 0, 201U/ml huIL-2 (Miltenyi Biotec, 130-097-743) on day 5 to day 14, and were then isolated using tetramer-APC (Proimmune, peptide code 357B) and anti-APC MicroBeads (Miltenyi Biotec, 130-090-855) and further expanded by stimulation by phytohemagglutinin (PHA) (1 μg/ml) and irradiated allogenic PBMC feeder cells (40 Gray, T-cells: irradiated allogenic PBMC=1:15) and huIL-2 (201U/ml for first 4 days, 2001U/ml for day 5 to day 14). T-cells (40% tetramer-positive) were resuspended at 10⁶ cells/ml and plated in a U-bottom 96-well tissue culture dish at 100 μl/well. Tetramer staining was performed according to manufacturer's protocol. Five (5) μl of tetramer was used per 10⁶ cells. The cells were incubated with tetramer at 37° C. for 2 hours in the dark.

For assay setup, 100 μl of DC (1×10⁵ cells) were added to 1×10⁵ T-cells and 0.1 μg/ml HA307 peptide was added to the DC and T-cell mixture. On day 6, tetramer-positive and cell expansion was detected by flow cytometry.

### Results

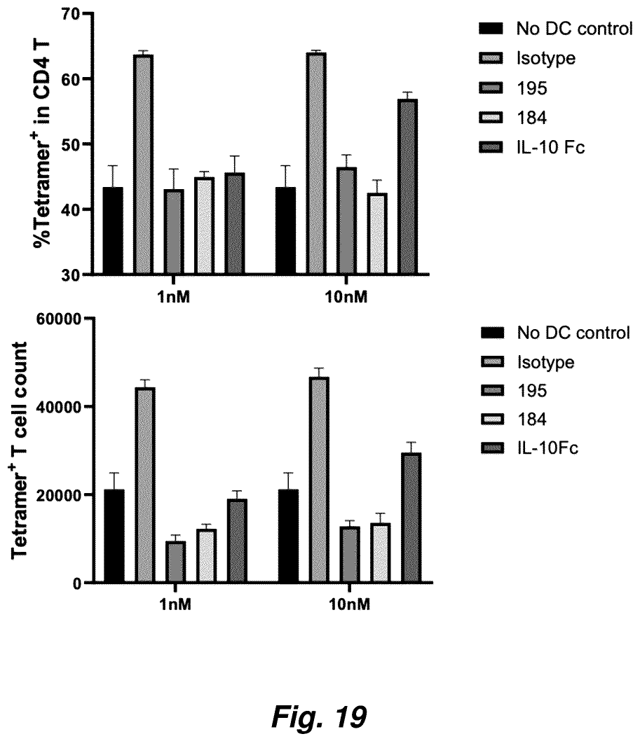

Dendritic cells were co-cultured with polyreactive antigen specific T cells in the presence of influenza antigen hemagglutinin peptide (HA307). The data are depicted in FIG. 19. The data are consistent with the conclusion that VHH1-VHH2 bispecific molecules 184 and 195 inhibited antigen specific T-cell frequency and cell number. Antigen specific T-cells were quantified by dextramer staining.

### Example 17

### Anti-IL-10Rα p VHH1-VHH2-Fc Molecules Dose-Dependently Inhibit LPS-Induced Cytokine Release In Vivo

In this example, we performed mouse experiments to measure how anti-IL-10Rαβ VHH1-VHH2-Fc bispecific molecules and IL-10-Fc regulates proinflammatory cytokine release by LPS stimulation.

### Methods

LPS-induced sepsis model. Humanized hIL-10Rα/hIL-10Rβ mice were generated in C57BL/6 mice and purchased from Biocytogen. Sepsis was induced in 6-week-old female mice by intraperitoneal injection of 100 μl of LPS derived from E. coli O111:B4 (Invivogen Cat No. vac-3pelps) diluted in PBS at 4 mg/kg dose, in combination with a single injection of 100 μl of PBS, IL-10-Fc or VHH1-VHH2 bispecific molecules 195 and 184 at different titrations. Blood was drawn from mice at 4 hours after LPS injection. Serum was isolated by centrifugation at 2000×g for 10 min at 4° C., and TNFα, IL-6, IL-17 and IL-12p70 were measured by Meso Scale Discovery (MSD) cytokine assay (mesoscale.com) per the manufacturer's instructions.

### Results

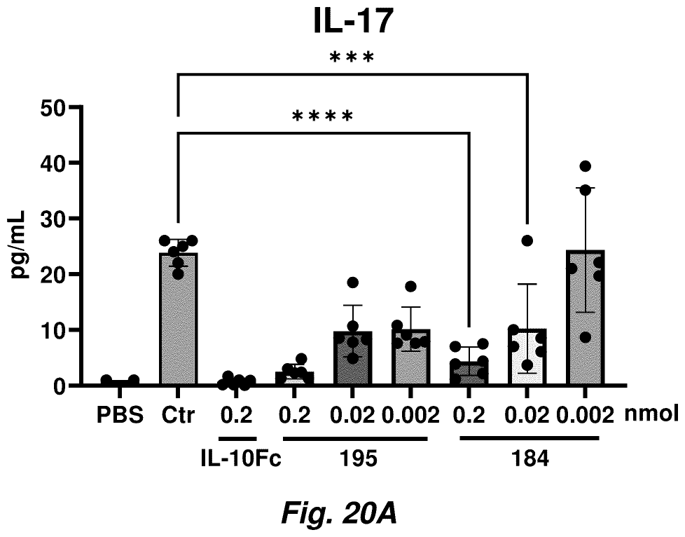

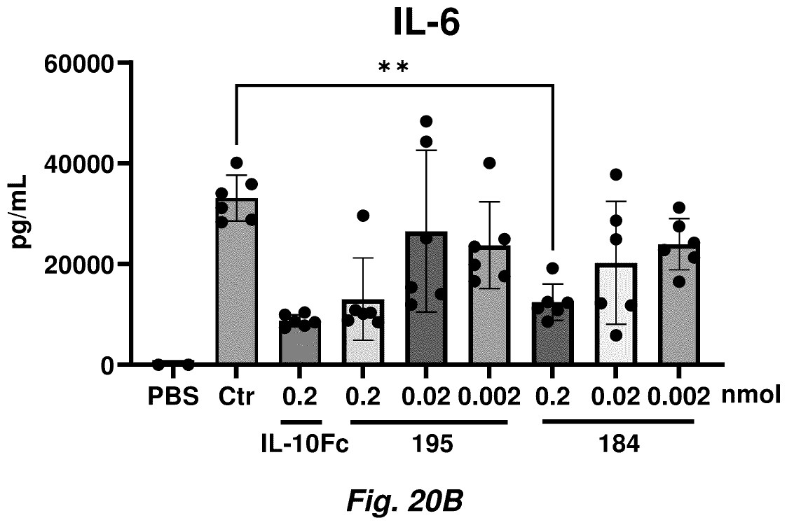

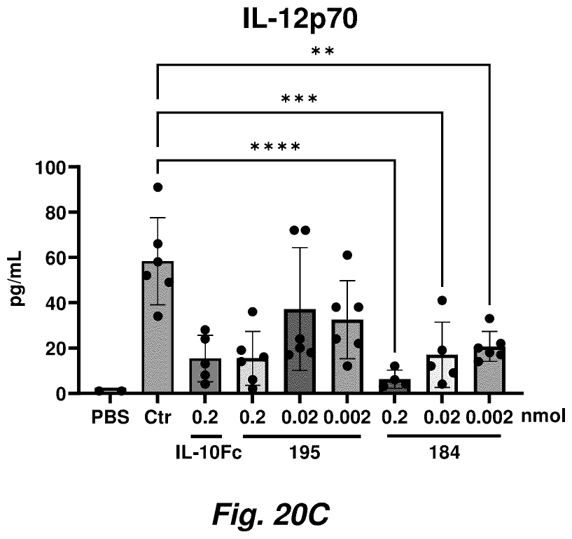

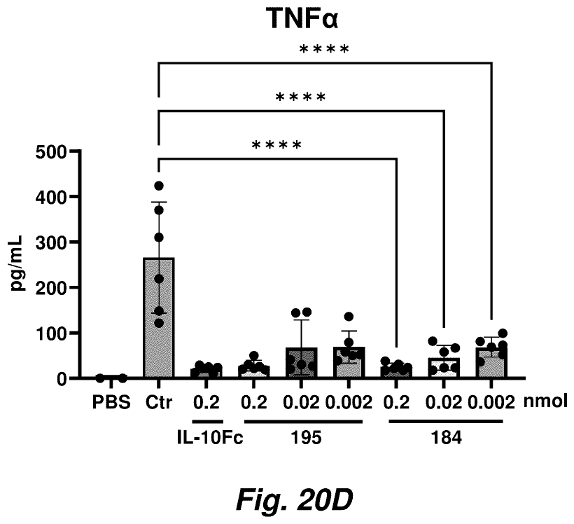

The results are depicted in FIGS. 20A (IL-17), 20B (IL-6), 20C (IL-12p70) and 20D (TNFα). The data in FIGS. 20A-20D are consistent with the conclusion that VHH1-VHH2 bispecific molecules 195 and 184 significantly reduced IL-17, IL-6, IL-12p70, and TNFα release in serum.

### Example 18

### Anti-IL-10Rαβ VHH1-VHH2-Fc Bispecific Molecules Are Efficacious in DSS Colitis

In this example, we tested the efficacy of anti-IL-10Rαβ VHH1-VHH2 bispecific variants in a mouse model of dextran sulfate sodium (DSS)-induced colitis.

### Methods

Dextran sulfate sodium induced colitis model. Ten-week-old female hIL-10Rα/hIL-10Rβ mice were given 2% dextran sulfate sodium (DSS) in their drinking water for 7 days to induce colitis and dosed three times a week with isotype control, IL-10-Fc, or VHH1-VHH2 bispecific molecule 877 (SEQ ID NO: 595) at 1 nmol per mouse, or IL-22-Fc at 0.35 nmole per mouse. Body weight was recorded daily from day 0 to day 7. Disease activity index was also evaluated and included daily measurement of body weight, evaluation of stool consistency, and presence of hemoccult. Colon tissue samples were collected on day 7 to determine colon length and weight. For gene quantification, colon tissue was cleaned, snap frozen in RNAprotect reagent (Qiagen Cat No. 76104) and stored at −80° C. Total cellular RNA was isolated by homogenizing tissue with gentleMACS M Tubes (Miltenyi Cat No. 130093236) and purification with RNeasy mini kit from Qiagen. Gene quantification was done using TaqMan RNA-to-CT 1-Step kit (Thermofisher Cat No. 4392938) on a QuantStudio 7 Pro machine according to the manufacturer's instructions. Spleen were collected to determine the spleen neutrophil frequency. Briefly, spleen was collected and dissociated using gentleMACS C Tubes and spleen dissociation kit (Miltenyi Cat No. 130095926). Single cell suspension was then stained with the following antibodies for 20 minutes at 4° C.: TCRb BUV805 (BD Cat No. 748405), CD45 APC-Cy7 (BD Cat No. 557659), Ly-6G Alexa700 (Biolegend Cat No. 127622), CD11b BV421 (BD Cat No. 562605), B220 PerCP-Cy5.5 (BD Cat No. 552771).

After staining, cells were pelleted and washed once with PBS+0.5% BSA and resuspended to read on BD Fortessa.

### Results

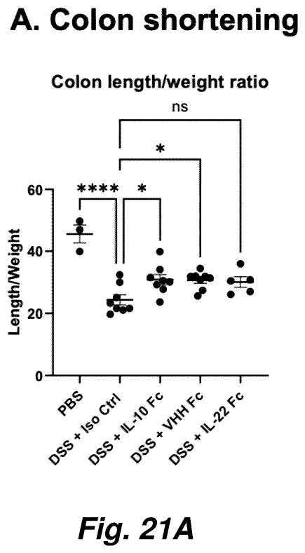

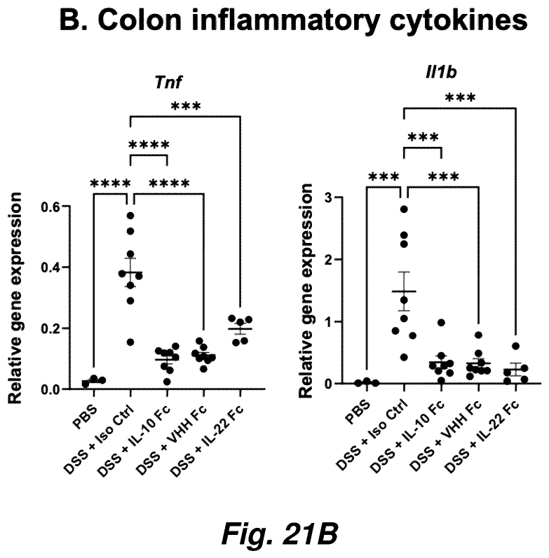

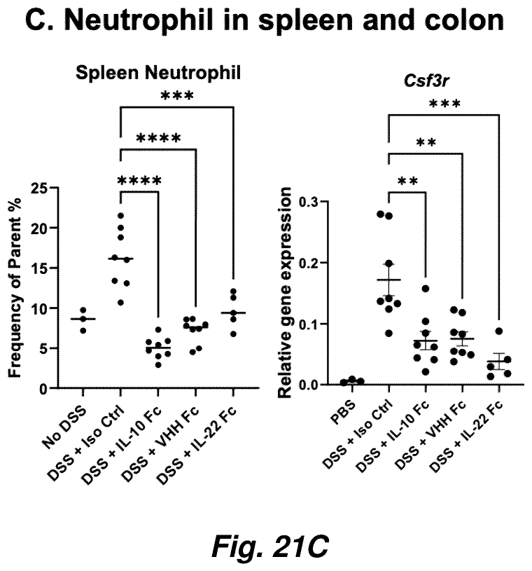

The results are provided in FIGS. 21A-21C. The data are consistent with the conclusion that herein described anti-IL-10Rαβ VHH1-VHH2 bispecific molecules, as represented by molecule 877, significantly reduced colon shortening, colon cytokine transcripts (e.g., Tnf, Il1b), and spleen (Ly6G+ cells) and colon (quantified by CSF3R transcript) neutrophils in a DSS colitis model. IL-22-Fc and IL-10-Fc were included as positive controls in this study.

### Example 19

### Pre-Anti-Drug Antibody (ADA) Assay

In this example, we assessed serum reactivity with four IL-10 receptor agonist VHH1-VHH2-Fc variants in a bridging ELISA assay to access the level of pre-existing anti-VHH antibodies in human populations. Illustrative VHH1-VHH2-Fc were expressed transiently in Expi293™ cells and purified using protein A and anion exchange chromatography.

### Methods

A bridging immunoassay was used to detect pre-existing ADA toward anti-IL-10Rαβ VHH1-VHH2-Fc molecules in human serum. This format requires bivalent pre-ADA binding to labeled VHH1-VHH2-Fc. Biotinylated VHH1-VHH2-Fc serves as the capture molecule binding to streptavidin coated Meso Scale Discovery (MSD) plate and the SULFO-TAG labeled VHH1-VHH2-Fc is the reporter.

Equal amounts of biotin and sulfo-tag labelled VHH1-VHH2-Fc 184, 183, 647 and 195 (SEQ ID NOs: 696, 694, 597 and 599, respectively) were prepared, each at 1 μg/mL concentration in ChonBlock blocking/sample dilution buffer (chondrex.com). Human serum from healthy volunteers was obtained from Bio-IVT for the assay. Thirty microliters of the testing antibody mixture were combined with 15 μL of 1:10 diluted human serum. The resulting solution was incubated overnight at 4° C. Following incubation, thirty microliters of the mixture was transferred to a pre-blocked streptavidin-coated Meso Scale Discovery (MSD) plate (L21SA) (mesoscale.com). The plate was further incubated for 2 hours with agitation at room temperature. After incubation, the plate was washed three times with 0.01% Tween 20/PBS buffer, then 40 μL of MSD read buffer was added to each well. The electro-chemiluminescent signal was then quantified using a Sector S 600 MSD plate reader. The assay was used to screen a total of 92 serum samples. The pre-ADA positive signal cut-point was determined using the sum of the median and 3×MAD (Median Absolute Deviation) of signals obtained from the 92 serum samples for each testing antibody. Caplacizumab-Fc was used as positive control in this assay.

### Results

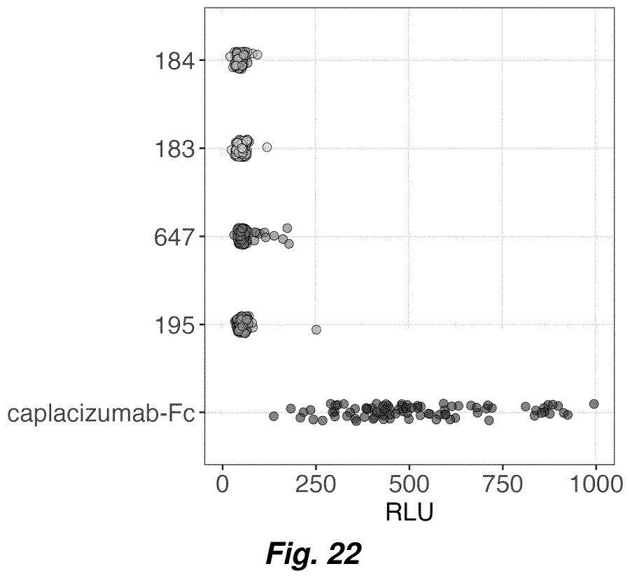

The results are depicted in FIG. 22. The results are consistent with the conclusion that, whereas the Caplacizumab-Fc positive control elicited a strong pre-existing ADA signal, VHH1-VHH2-Fc molecules 184, 183, 647 and 195 elicited a signal marginally above the cut-point line, demonstrating that all of these VHH-VHH-Fc have low risk of pre-existing ADA in healthy human volunteers.

### Example 20

### T-cell Immunogenicity Assay

In this example, we evaluated four representative anti-IL-10Rαβ agonist VHH1-VHH2-Fc variants (195, 647, 183 and 184) in T-cell proliferation assay to assess the level of T-cell immunogenicity in human populations. Illustrative VHH1-VHH2-Fc were expressed transiently in Expi293™ cells and purified using protein A and anion exchange chromatography.

### Methods

Cryopreserved PBMCs from 48 donors were purchased from BioIVT and HLA typed. Twenty μl of test articles at a concentration of 2.5 mg/ml and unstimulated controls (media alone) were plated in triplicate in tissue culture (TC)-treated flatbottom 24-well plates (Corning Costar). Two million PBMCs were added per well in 1 ml volume using the Biomek i7 liquid dispenser (Beckman Coulter), resulting in a final test article concentration of 0.05 mg/ml. Plates were then incubated for 7 days at 37° C. and 5% CO₂ in an incubator (Thermo Fisher). On day 6, cells were treated with BrdU (BD Biosciences) at a final concentration of 10 μM.

On day 7, cells were transferred to 96-well V-bottom plates (Corning Costar) using the Biomek i7 for staining and flow cytometry analysis. Cells were incubated with Dylight-800 NHS Ester (Thermo Scientific) for 20 minutes to identify live cells, followed by washing with FACS wash Buffer (DPBS+3% FBS) using an EL406 Washer/Dispenser (Agilent). Cells were then treated with Human Fc block (Biolegend) for 10 min at 40° C. and stained with antibodies against CD3, CD4 and CD8 (Biolegend) for 30 min at 40° C. using the EL406 Washer/Dispenser (Biotek). Cells were then washed 4 times with FACS buffer using a EL406 washer to remove unbound antibodies.

Cells were then fixed and permeabilized with the BD Cytofix/Cytoperm buffer (BD Biosciences) for 20 min at 40° C., washed once with BD Perm/Wash buffer (BD Biosciences) using the Biotek EL406, and treated with BD cytoperm permeabilization Buffer Plus for 10 min at 40° C. After another wash with BD Perm/Wash buffer, cells were fixed for 5 min at room temperature with BD Cytofix/Cytoperm buffer. Cells were then washed 1× with BD Perm/Wash buffer (Biotek EL406) and are treated with DNase (300 μg/ml) (Sigma) for 1 hour at 37° C. Following a wash (Biotek EL406) with BD Perm/Wash buffer, anti-BrdU antibody (Biolegend) was added to the cells and incubated at room temperature for 20 min to identify actively dividing cells. Cells were washed once (Biotek EL406) with BD Perm/Wash buffer and re-suspended in PBS (Gibco). Cells were then acquired on an Attune NxT Cytometer (Thermo Fisher) to quantify BrdU+ cells.

The gating strategy used to identify BrdU+ cells was as follows: size gate was applied to remove cell debris and cells were identified using forward and side-light scatter. Singlets were then gated for live cells followed by gating on CD3+CD4+ cells. The percentage of BrdU+ cells within CD3+CD4+ cells were then used from each tested condition to calculate the stimulation index.

The stimulation index was calculated for each test article and donors. The Percentage of BrdU+ cells within the CD3+CD4+ population was quantified and averaged by geometric means for both the unstimulated control wells and test article wells. Stimulation index for each test article in each donor was calculated with the following equation:

Stimulation Index (SI)=geo means of Test Article/geo means of Unstimulated

The stimulation index of unstimulated wells was normalized to 1. A donor was considered as a positive responder if SI for a test article was greater than 2.0. The response rate was computed for each test article using the following equation:

Response Rate=Number of responding donors/total number of donors

Avastin (bevacizumab) was used as negative control and both bococizumab and keyhole limpet hemocyanin (KLH) protein were used as positive controls in this assay.

### Results

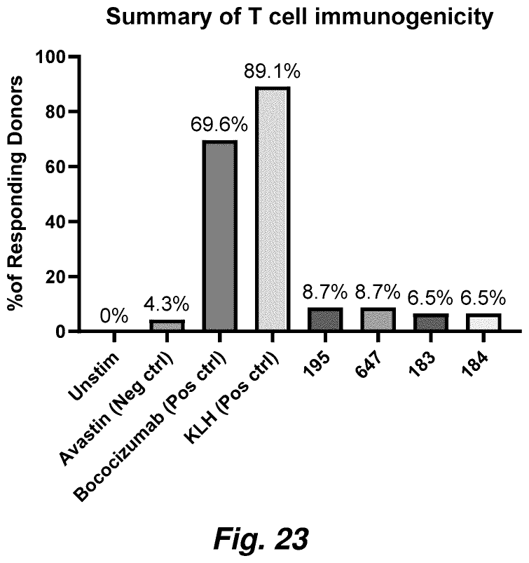

Four representative humanized anti-IL-10Rαβ agonist VHH1-VHH2-Fc variants (195, 647, 183 and 184) were expressed, purified, and tested in a T-cell proliferation assay from a panel cryopreserved PBMCs from 48 donors. All four tested VHH1-VHH2-Fc demonstrated a low T-cell stimulating propensity with 6.5%-8.7% of donors have SI greater than 2. The results are depicted in FIG. 23. The results are consistent with the conclusion that representative humanized VHHT-VHH2-Fc variants 195, 647, 183 and 184 elicit minimal T-cell immunogenicity of human PBMCs.

### Example 21

### Single Dose Pharmacokinetics of Anti-IL-10Rαβ Agonist VHH1-VHH2-Fc Molecules after Intravenous Administration in Cynomolgus Monkeys

In this example, we compared the single dose pharmacokinetics (PK) of two representative anti-IL-10Rαβ agonist VHH1-VHH2-Fc molecules in cynomolgus macaques.

### Methods

Two anti-IL-10Rαβ agonist VHH-VHH-Fc molecules (195 and 184) were administered to naïve cynomolgus macaques (n=2/group, 1M+1F) (Covance, WI) at 100 mg/kg via a single intravenous (IV) bolus to characterize their PK profiles. Serial plasma samples collected from monkeys were analyzed using a selective bioanalytical method of sufficient sensitivity to determine plasma concentration-time profiles and mean plasma PK parameters by non-compartmental analysis (NCA). The bioanalytical method utilized an anti-human IgG monkey adsorbed antibody (Southern Biotech, AL) as a capture reagent and SULFO-TAG™ (MesoScale Discovery, MD) labeled human IL-10Rα as a secondary reagent for electrochemiluminescence (ECL) detection on a Mesoscale Discovery Quickplex SQ 120 plate reader. The calibration curve used the respective individual IL-10R agonists as reference standards in spiked macaque matrix fit to a 4-parameter logistic model with 1/Y2 weighting. Analyte concentrations were determined from the ECL signals back-fitted to the calibration curve. Plasma concentration-time profiles were used to calculate the mean plasma PK parameters by NCA.

### Results

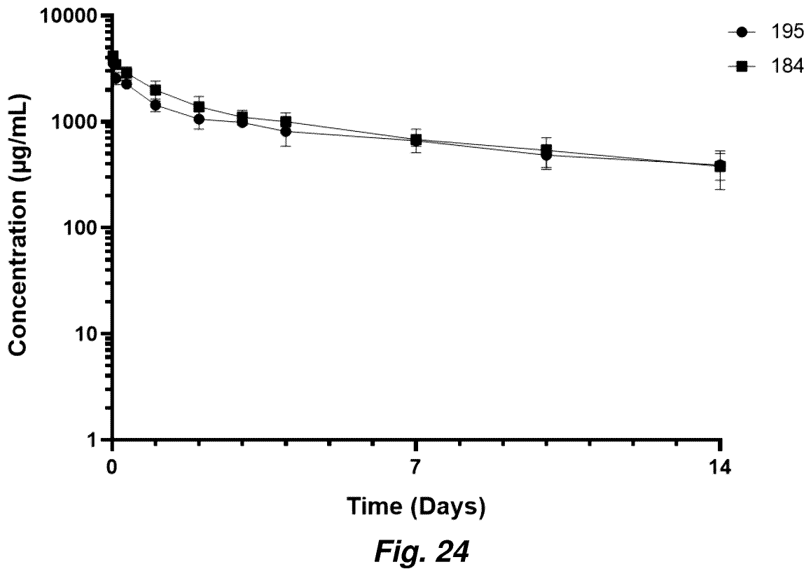

PK analysis demonstrated that anti-IL-10Rαβ agonist VHH1-VHH2-Fc molecules 195 and 184 had IgG-like PK following IV administration in cynomolgus macaques. Both VHH1-VHH2-Fc 195 and 184 have similar PK as exemplified by the calculated NCA PK parameters in Table 25. The plasma concentration-time profiles are shown in FIG. 24.

TABLE 25
Non-Compartmental Analysis (NCA) of Anti-IL-10Rαβ Bispecific VHH1-
VHH2-Fc After Single IV Bolus Administration to Naïve Cynomolgus Macaques

| Mol. | Dose | | Cₘₐₓ | AUC₀₋ₗₐₛₜ | CL | Vₛₛ | Half-life |
|---|---|---|---|---|---|---|---|
| name | (mg/kg) | N | (μg/mL) | (μg*day/mL) | (mL/day/kg) | (mL/kg) | (days) |
| 195 | 100 | 2 | 3550 | 10900 | 6.35 | 73.2 | 9.21 |
| 184 | 100 | 2 | 4150 | 12900 | 6.00 | 58.3 | 8.25 |

AUC₀₋ₗₐₛₜ = Area under the curve from time zero to time at last measurable concentration; CL = clearance; Vss = volume of distribution at steady state.

### Example 22

### Differential Requirement for TYK2 Phosphorylation Induced by Anti-IL-10Rαβ Bispecific VHH1-VHH2-Fc and Wild-type IL-10 for STAT3 Phosphorylation (pSTAT3) and IL-10-Mediated Immune Suppressive Function

### Methods

Peripheral blood mononuclear cells (PBMCs) were isolated from the blood of healthy volunteers using Lymphoprep medium. Monocytes were isolated using the Miltenyi Human Classical Monocyte Isolation Kit, and macrophage differentiation was induced by culturing monocytes in the presence of human GM-CSF.

PBMCs from two individual donors were seeded at a density of 20,000 cells per well in 190 μL of RPMI-based medium (RPMI supplemented with GlutaMAX and 10% fetal bovine serum) in a 96-well flat-bottom plate. Cells were pretreated with a TYK2 inhibitor (final concentration: 1.05 μM) for 30 minutes at 37° C. in a humidified incubator with 5% CO₂. Following pretreatment, cells were stimulated with lipopolysaccharide (LPS; Invivogen tlrl-eblps, 5 μL added to achieve a final concentration of 10 ng/mL) and treated with interleukin-10 (IL-10; 5 μL added to achieve a final concentration of 50 nM). Incubation was continued for 20 hours under the same conditions. Cell culture supernatants were then collected and analyzed for cytokine levels using the Meso Scale Discovery (MSD) Human Proinflammatory Panel II, with samples diluted two-fold prior to measurement.

### Results

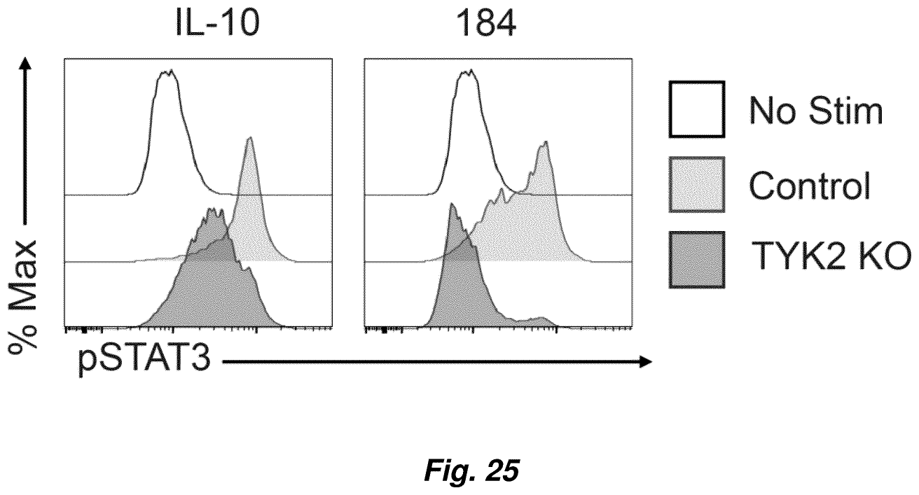

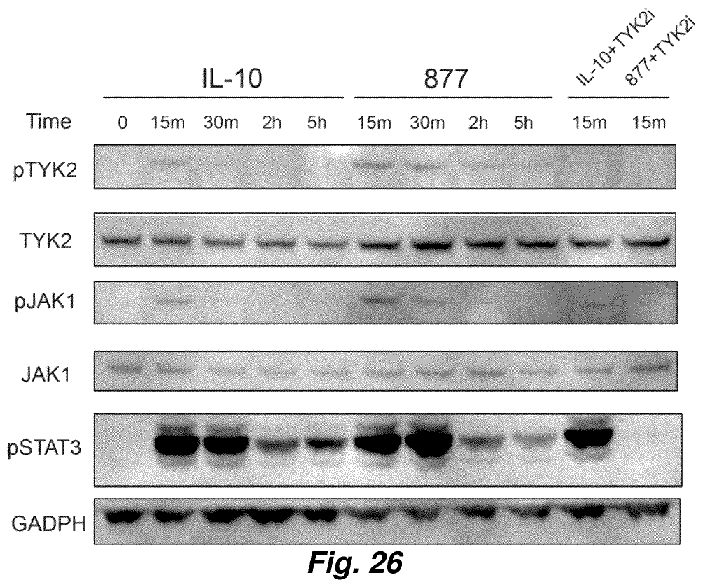

Unlike dimeric IL-10, which induces Janus kinase 1 (JAK1; NCBI Gene ID: 3716) and tyrosine kinase 2 (TYK2; NCBI Gene ID: 7297) phosphorylation upon tetrameric IL-10RA/IL-10RB crosslinking, myeloid-selective anti-IL-10Rαβ bispecific VHH1-VHH2-Fc molecules (e.g., 877, 184) exhibited a distinct receptor crosslinking mechanism. CRISPR knockout of TYK2 reduced, but did not eliminate, IL-10-mediated STAT3 phosphorylation in human macrophages (FIG. 25). In contrast, molecule 184 strictly required TYK2 for STAT3 phosphorylation (FIG. 25). Furthermore, wild-type IL-10 induced JAK1 and STAT3 phosphorylation in the presence of a TYK2 inhibitor, whereas molecule 877 failed to trigger JAK1 and STAT3 phosphorylation under the same conditions (FIG. 26).

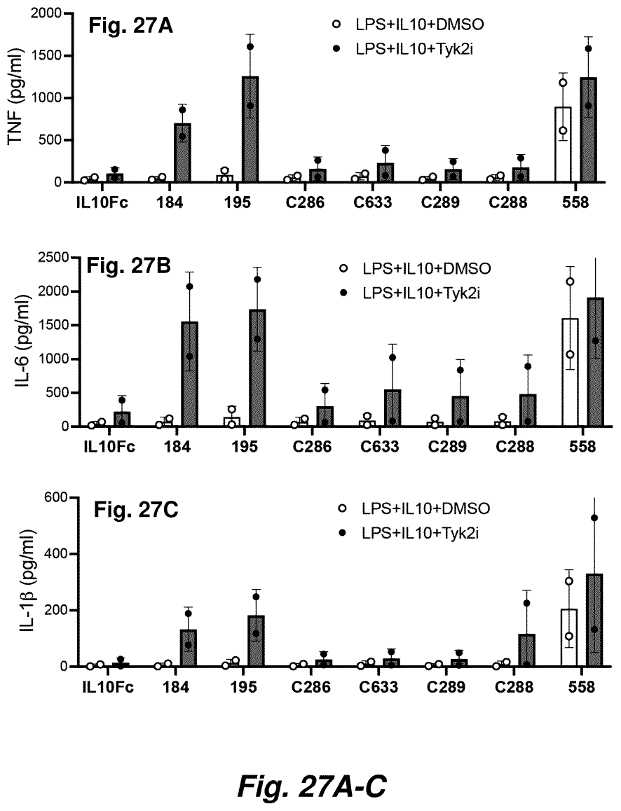

We further evaluated the suppressive function of myeloid-selective anti-IL-10Rαβ bispecific VHH1-VHH2-Fc molecules in the presence of a TYK2 inhibitor (Tyk2i). Molecules 184 and 195 required TYK2 kinase activity to suppress LPS-induced TNF and IL-6 production. In contrast, comparator molecules in VHH1-VHH2-Fc format (C286, C633, C289, C288) and IL-10-Fc suppressed TNF and IL-6 production even in the presence of the TYK2 inhibitor (FIGS. 27A-27C). Isotype control 558 did not suppress TNF or IL-6 production in the presence or absence of the TYK2 inhibitor. Taken together, these data are consistent with the conclusion that myeloid-selective IL-10 VHH-VHH-Fc molecules, including 184, 195, exhibit distinct TYK2-dependent signaling compared to native IL-10 and comparator molecules, which is associated with their myeloid-selective activity and function.

### Example 23

### Comparative Analysis of Anti-IL-10Rαβ Bispecific VHH1-VHH2-Fc and Comparator Molecules in IL-10R pSTAT3 Reporter Assay Demonstrates a Distinct Profile of Tyk2 Dependency

In this example, we evaluated anti-IL-10Rαβ agonist VHH1-VHH2-Fc molecules 184, 195, and comparator molecules in TYK2 knockout IL10R pSTAT3 reporter assay to determine the dependency of TYK2 in pSTAT3 activation.

### Method

HEK293 IL-10R pSTAT3 luciferase reporter cell line was developed by overexpressing the human IL-10Rα, human IL-10Rβ, and a Firefly luciferase gene driven by STAT3 response elements in HEK293T cells. The TYK2 knockout IL10R-pSTAT3 luciferase reporter cell line was generated using CRISPR RNP technology to abolish the TYK2 gene. The IL-10 receptor agonist activity of VHH1-VHH2-Fc molecules 184, 195, and a panel of comparator VHHs was evaluated in both HEK293T IL-10R-pSTAT3 luciferase reporter and HEK293T TYK2 KO IL-10R-pSTAT3 luciferase reporter assay. One day before the assay, culture medium was used to prepare sample plate with eight points dose-response (1:5 serial dilution). Recombinant human IL-10 (50 ng/mL PeproTech Cat #200-10) was used as a positive control. Reporter cells were detached from plate using TrypLE™ (Thermo Fisher Scientific) and washed with cell culture media. Cell concentration was adjusted to 8×10⁴ cells/ml and 25 μL (2K cells) of cell suspension was added into each well of solid white tissue culture (TC) 384-well plate (Greiner Cat #781080). On the day of the assay, five microliters (5 μL) of VHH sample were added to the cell plate and incubated at 37° C. for 3 hours. Thirty microliters (30 μL) of Bio-Glo™ Reagent (Promega Cat #G7941) were added into each assay well, followed by a brief spin at 1000 rpm. The 384-well plate was then incubated at room temperature for 10 mins and read by EnVision plate reader (Revvity).

The luminescence readout for VHHs treated wells was normalized to average signal from positive (IL-10) and negative (media) controls and expressed as % IL-10R agonist:

$$\% IL - 10 R agonist = \frac{( ( Luminescence sample - Luminescence 0 \% activity )}{( Luminescence 100 \% activity - Luminescence 0 \% activity ) ) * 100}$$

Where 0% IL-10R agonist=Signal in wells with media control
100% IL-10R agonist=Signal in wells with IL-10 (50 ng/mL)

EC₅₀ values were calculated based on log concentration of drug and percent of IL-10 signal using a 4-parameter variable slope nonlinear regression model and least square curve fit (Gilead Sciences, Foster City, CA).

$$y = \frac{100 \times E {C}_{50}^{m}}{E {C}_{50}^{m} + {[ A ]}^{m}}$$

where y=% Activity, m=Hill coefficient, and [A]=Agonist concentration.
EC₅₀ values are derived from the average of duplicate data points for each antibody concentration.
Eₘₐₓ is defined as the maximum normalized percentage of IL-10R agonist achieved in the assay.

### Results

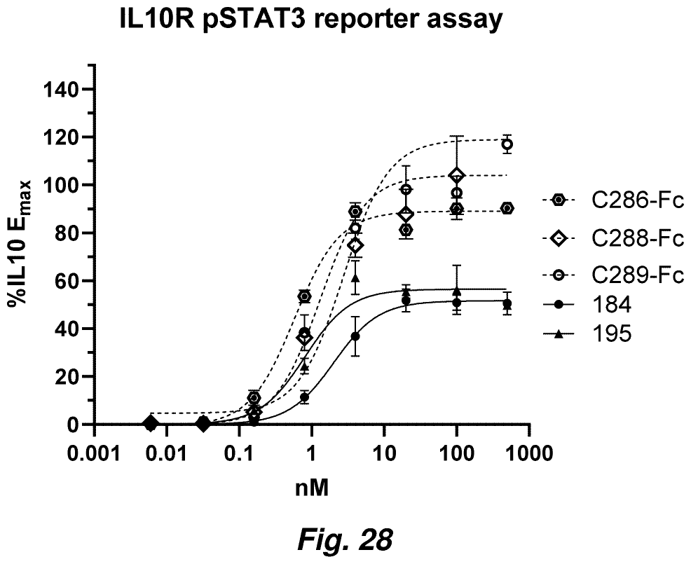

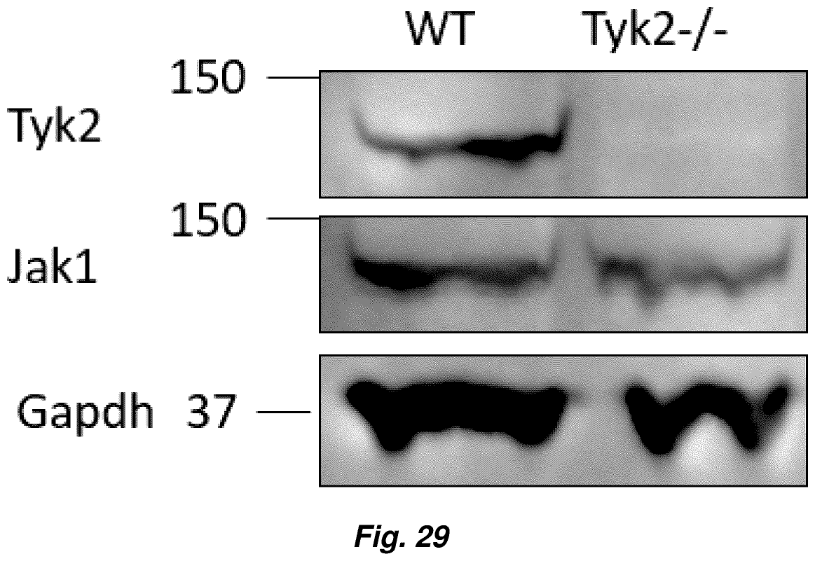

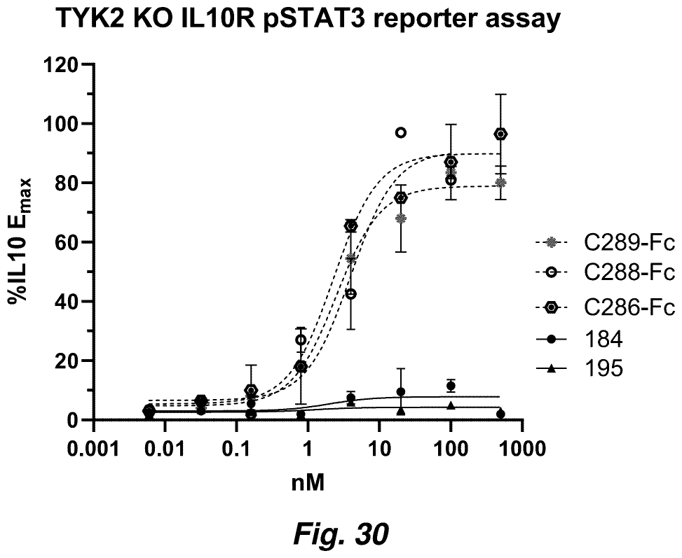

The EC₅₀ and Emax values are summarized in Table 26 and FIGS. 28-30. All Fe-fused variants of the molecules exhibited potent IL-10 receptor agonist activity, effectively inducing pSTAT3 luciferase reporter gene expression. Anti-IL-10Rαβ agonist VHH1-VHH2-Fc molecules 184 and 195 demonstrated comparable potency to the comparator molecules, but elicited significantly lower Emax responses. Notably, in TYK2 knockout IL-10R pSTAT3 reporter cells, both molecules 184 and 195 showed markedly lower agonist activity compared to the comparator molecules, suggesting that their activity is TYK2-dependent. In contrast to comparator molecules, anti-IL-10Rαβ agonist VHH1-VHH2-Fc molecules 184 and 195 did not efficiently initiate JAK1 transactivation in the HEK293T cells used in this assay.

TABLE 26
Comparative Potency Analysis of Anti-IL-10Rαβ
Bispecific VHH1-VHH2-Fc and Comparator Molecules
in HEK293 IL-10R pSTAT3 Luciferase Reporter Cell Lines

| | | | | TYK2 | TYK2 |
|---|---|---|---|---|---|
| | | | | KO | KO |
| | | IL10R | IL10R | IL10R | IL10R |
| | | pSTAT3 | pSTAT3 | pSTAT3 | pSTAT3 |
| Molecule | | EC₅₀ | Eₘₐₓ % | EC₅₀ | Eₘₐₓ % |
| ID | Format | nM | IL10 | nM | IL10 |
| 195 | VHH1-VHH2-Fc | 0.82 | 56% | 1.23 | 9% |
| | bivalent | | | | |
| 184 | VHH1-VHH2-Fc | 2.02 | 52% | 2.01 | 5% |
| | bivalent | | | | |
| C286 | VHH1-VHH2-Fc | 0.60 | 89% | 2.28 | 96.5% |
| | bivalent version | | | | |
| | of C301 | | | | |
| C289 | VHH1-VHH2-Fc | 1.46 | 101% | 2.50 | 83.5% |
| | bivalent version | | | | |
| | of C382 | | | | |
| C288 | VHH1-VHH2-Fc | 1.12 | 98% | 3.99 | 97% |
| | bivalent version | | | | |
| | of C383 | | | | |

### Example 24

### Robust Therapeutic Efficacy of IL-10R Agonism in Preclinical Models of Arthritis and Inflammatory Dermatitis

To evaluate the therapeutic potential of IL-10 receptor (IL-10R) agonism, we tested IL-10-Fc in preclinical models of arthritis and inflammatory dermatitis. Since IL-10 VHH-VHH-Fc does not bind to endogenous IL-10RA or IL-10RB, IL-10-Fc was used as a mouse surrogate to assess the therapeutic efficacy of IL-10R agonism in inflammatory disease models.

### Methods

Collagen-Induced Arthritis (CIA) Model (See, e.g., Brand, et al., Nat Protoc (2007 2(5):1269-75; Rosloniec, et al., Curr Protoc (2021) 1(12):e313). In the mouse collagen-induced arthritis model, animals were subcutaneously injected with an emulsion of chicken collagen II and complete Freund's adjuvant (CFA) on day 0. On day 18, mice received a booster injection of chicken collagen II emulsified in incomplete Freund's adjuvant (IFA). Animals were grouped, and therapeutic dosing with IL-10-Fc or anti-IL-17A antibody began on day 24 and continued until study termination on day 48. Animals were administered active or control agent three times per week with 50 μg of IL-10-Fc, 50 μg of a control antibody, or 500 μg of anti-IL-17A antibody. The mean arthritic score, based on paw and ankle swelling and ankylosis, was recorded over time. See, e.g., Pietrosimone, et al., Bio Protoc. (2015) 5(20):e1626; Luan, et al., Exp Ther Med. (2021); 22(3):93. Naïve animals did not receive any active or control agent.

Mannan-Induced Dermatitis Model (See, e.g., Khmaladze, et al., Proc Natl Acad Sci USA. (2014) 111(35):E3669-78). B10.RIII mice were intraperitoneally (IP) injected with 25 mg of mannan on days 0 and 7, inducing clinical signs of dermatitis on the paws and ears, which were scored throughout the study. Animals were grouped, and therapeutic dosing with IL-10-Fc or anti-IL-17A antibody began on day 6. Animals received a single dose of 5 μg of IL-10-Fc or 50 μg of a control antibody on day 9, or 500 μg of anti-IL-17A antibody on days 8 and 10. Ear dermatitis were scored daily starting on day 3. See, e.g., Schwartz, et al., Allergy (2019) 74(10):1920-1933; Riedl, et al., Int J Mol Sci. (2023) 24(15):12325. Scores represent the average total dermatitis score for ears for each group.

### Results

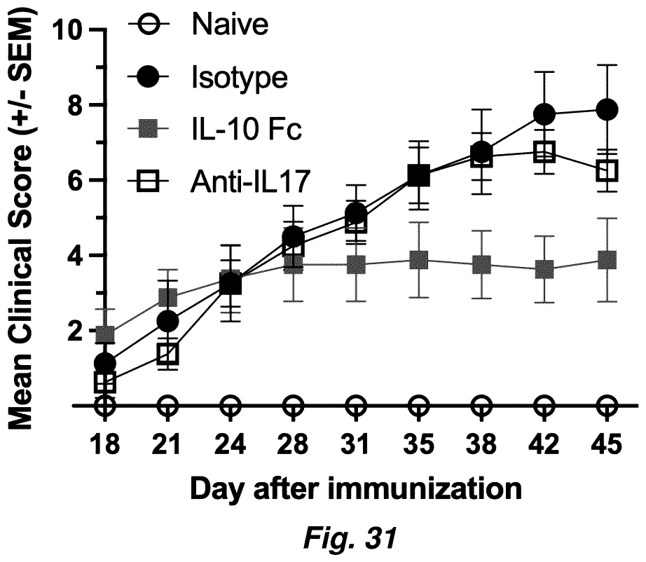

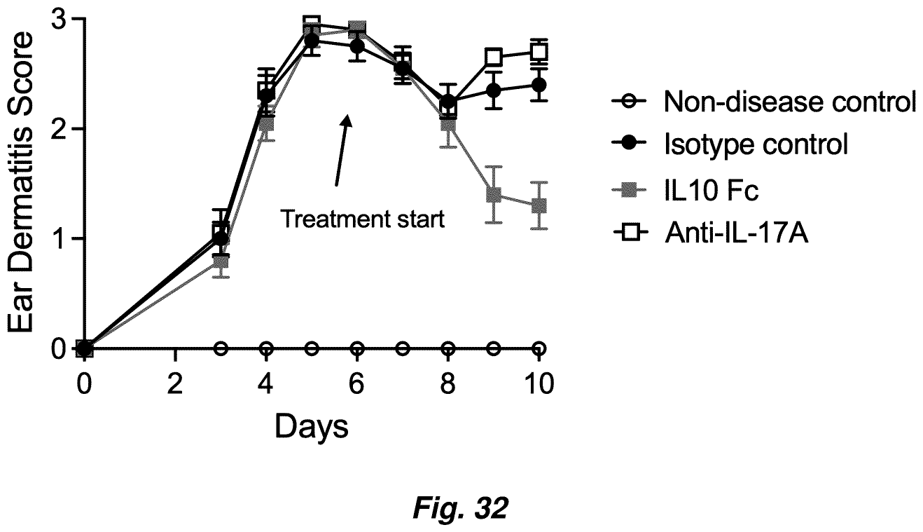

IL-10-Fc demonstrated robust therapeutic efficacy in preclinical models of arthritis (FIG. 31) and inflammatory dermatitis (FIG. 32). These data are consistent with the conclusion that the development of IL-10 VHH-VHH-Fc for inflammatory diseases, including rheumatoid arthritis and inflammatory dermatitis indications such as psoriasis and hidradenitis suppurativa (HS). Anti-IL-17 antibody was included as an active comparator for these studies.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A single domain antibody (sdAb) comprising a variable domain (VHH) that specifically binds to the extracellular domain of interleukin-10 receptor (IL-10R) alpha subunit (IL-10Rα), the VHH comprising a complementarity determining region (CDR) 1, a VHH-CDR2, and a VHH-CDR3 comprising the following amino acid sequences, according to Kabat, respectively:
1) SEQ ID NOs: 1, 2 and 3; (A01)
2) SEQ ID NOs: 4, 5 and 6; (A06)
3) SEQ ID NOs: 7, 8 and 9: (A09)
4) SEQ ID NOs: 10, 11 and 12; (B07)
5) SEQ ID NOs: 4, 13 and 14; (B08)
6) SEQ ID NOs: 4, 15 and 16; (C09)
7) SEQ ID NOs: 4, 17 and 18; (D08)
8) SEQ ID NOs: 19, 20 and 21; (D14)
9) SEQ ID NOs: 22, 23 and 24; (E07)
10) SEQ ID NOs: 22, 25 and 27; (huE07)
11) SEQ ID NOs: 22, 27 and 24; (huE07)
12) SEQ ID NOs: 4, 28 and 29; (E15)
13) SEQ ID NOs: 4, 30 and 31; (F09)
14) SEQ ID NOs: 22, 23 and 32; (F10)
15) SEQ ID NOs: 33, 34 and 35; (F11)
16) SEQ ID NOs: 4, 36 and 29; (F21)
17) SEQ ID NOs: 19, 37 and 38; (G22)
18) SEQ ID NOs: 39, 40 and 41; (G23)
19) SEQ ID NOs: 42, 43 and 44; (H22)
20) SEQ ID NOs: 45, 46 and 47; (I04)
21) SEQ ID NOs: 4, 48 and 6; (J13)
22) SEQ ID NOs: 4, 49 and 50; (J24)
23) SEQ ID NOs: 19, 51 and 52; (L05)
24) SEQ ID NOs: 19, 37 and 53; (11) or
25) SEQ ID NOs: 54, 55 and 24 (N16).

2-6. (canceled)

7. The sdAb of claim 1, wherein the VHH comprises the amino acid sequence of any one of SEQ ID NOs: 212, 230, 233, 234, 249 and 710, or an amino acid sequence that is at least 80%, at least 85%, at least 90%, at least 91%, at least 92%, at least 93%, at least 94%, at least 95%, at least 96%, at least 97%, at least 98%, or at least 99% identical, to one of the amino acid sequences of SEQ ID NOs: 212, 230, 233, 234, 249 and 710.

8. The sdAb of claim 1, wherein the VHH comprises the amino acid sequence of any one of 210-211, 213-229, 231-232, 235-248, 250-287 and 752-755, or an amino acid sequence that is at least 80%, at least 85%, at least 90%, at least 91%, at least 92%, at least 93%, at least 94%, at least 95%, at least 96%, at least 97%, at least 98%, or at least 99% identical, to one of the amino acid sequences of SEQ ID NOs: 210-211, 213-229, 231-232, 235-248, 250-287 and 753-755.

9-17. (canceled)

18. A single domain antibody (sdAb) comprising a variable domain (VHH) that specifically binds to the extracellular domain of interleukin-10 receptor (IL-10R) beta subunit (IL-10Rβ), the VHH comprising a complementarity determining region (CDR) 1, a VHH-CDR2, and a VHH-CDR3 comprising the following amino acid sequences, according to Kabat, respectively:
1) SEQ ID NOs: 288, 289 and 290; (A02)
2) SEQ ID NOs: 291, 292 and 293; (A05)
3) SEQ ID NOs: 291, 294 and 295; (huA05)
4) SEQ ID NOs: 296, 297 and 298; (huA05)
5) SEQ ID NOs: 299, 300 and 301; (B10)
6) SEQ ID NOs: 302, 303 and 304; (B12)
7) SEQ ID NOs: 305, 306 and 307; (C01)
8) SEQ ID NOs: 296, 308 and 309; (C04)
9) SEQ ID NOs: 310, 311 and 312; (C10)
10) SEQ ID NOs: 302, 303 and 313; (D01)
11) SEQ ID NOs: 314, 315 and 316; (E08)
12) SEQ ID NOs: 317, 318 and 319; (E10)
13) SEQ ID NOs: 320, 321 and 322; (G01) or
14) SEQ ID NOs: 323, 324 and 325 (G02).

19-22. (canceled)

23. The sdAb of claim 18, wherein the VHH comprises the amino acid sequence of any one of SEQ ID NOs: 448, 449, 455 and 460, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 448, 449, 455 and 460.

24. The sdAb of claim 18, wherein the VHH comprises the amino acid sequence of any one of SEQ ID NOs: 446-447, 450-454, 456-459 and 461-474, or an amino acid sequence that is at least 80%, at least 85%, at least 90%, at least 91%, at least 92%, at least 93%, at least 94%, at least 95%, at least 96%, at least 97%, at least 98%, or at least 99% identical, to the full length of the amino acid sequence of any one of SEQ ID NOs: 446-447, 450-454, 456-459 and 461-474.

25-36. (canceled)

37. A bispecific antibody comprising a first single immunoglobulin variable domain (VHH) and a second VHH, wherein the first VHH specifically binds to the extracellular domain of interleukin-10 receptor (IL-10R) alpha subunit (IL-10Rα) and the second VHH specifically binds to the extracellular domain of interleukin-10 receptor beta subunit (IL-10Rβ), wherein the bispecific antibody is an IL-10R agonist that predominantly or selectively suppresses activation of myeloid cells and induces a maximum effect (Emax) activation of T cells or B cells that is 15% or less of wild-type IL-10.

38. A bispecific antibody comprising a first immunoglobulin variable domain (VHH) and a second VHH, wherein the first VHH specifically binds to the extracellular domain of interleukin-10 receptor (IL-R) alpha subunit (IL-10Rα) and the second VHH specifically binds to the extracellular domain of interleukin-10 receptor beta subunit (IL-10Rβ), wherein the bispecific antibody is an IL-10R agonist suppressing activation of myeloid cells with a half maximal effective concentration (EC₅₀) less than 10 pM and activating T cells and B cells below a predetermined threshold level at a concentration above 1000 nM.

39-56. (canceled)

57. The bispecific antibody of claim 38, wherein the first VHH comprises a first heavy chain variable domain (VHH1) comprising a VHH1-complementarity determining region (CDR) 1, a VHH1-CDR2, and a VHH1-CDR3 and the second VHH comprises a second heavy chain variable domain (VHH2) comprising a VHH2-complementarity determining region (CDR) 1, a VHH2-CDR2, and a VHH2-CDR3, comprising the following amino acid sequences, according to Kabat, respectively:
1) SEQ ID NOs: 22, 23, 24, 291, 292 and 293; (877)
2) SEQ ID NOs: 22, 25, 26, 291, 294 and 295; (647)
3) SEQ ID NOs: 22, 27, 24, 291, 297 and 298; (195)
4) SEQ ID NOs: 22, 23, 24, 288, 289 and 290; (048)
5) SEQ ID NOs: 22, 25, 26, 288, 289 and 290; (049)
6) SEQ ID NOs: 22, 27, 24, 288, 289 and 290; (050)
7) SEQ ID NOs: 22, 23, 24, 296, 308 and 309; (274)
8) SEQ ID NOs: 22, 25, 26, 296, 308 and 309; (771)
9) SEQ ID NOs: 22, 27, 24, 296, 308 and 309; (772)
10) SEQ ID NOs: 4, 28, 29, 288, 289 and 290; (054)
11) SEQ ID NOs: 4, 28, 29, 291, 294 and 295; (883)
12) SEQ ID NOs: 4, 28, 29, 291, 294 and 295; (784)
13) SEQ ID NOs: 4, 28, 29, 291, 297 and 298; (785)
14) SEQ ID NOs: 4, 5, 6, 288, 289 and 290; (046)
15) SEQ ID NOs: 4, 5, 6, 291, 294 and 295; (875)
16) SEQ ID NOs: 4, 5, 6, 291, 294 and 295; (776)
17) SEQ ID NOs: 4, 5, 6, 291, 297 and 298; (777)
18) SEQ ID NOs: 4, 5, 6, 296, 308 and 309; (272)
19) SEQ ID NOs: 4, 15, 16, 288, 289 and 290; (052)
20) SEQ ID NOs: 4, 15, 16, 296, 308 and 309; (278)
21) SEQ ID NOs: 4, 17, 18, 288, 289 and 290; (047)
22) SEQ ID NOs: 4, 17, 18, 291, 292 and 293; (876)
23) SEQ ID NOs: 4, 17, 18, 291, 294 and 295; (778)
24) SEQ ID NOs: 4, 17, 18, 291, 297 and 298; (779)
25) SEQ ID NOs: 45, 46, 47, 288, 289 and 290; (062)
26) SEQ ID NOs: 45, 46, 47, 291, 292 and 293; (891)
27) SEQ ID NOs: 45, 46, 47, 291, 294 and 295; (780)
28) SEQ ID NOs: 45, 46, 47, 291, 297 and 298; (781)
29) SEQ ID NOs: 45, 46, 47, 296, 308 and 309; (290)
30) SEQ ID NOs: 4, 48, 6, 291, 292 and 293; (892)
31) SEQ ID NOs: 4, 48, 6, 291, 294 and 295; (793)
32) SEQ ID NOs: 4, 48, 6, 291, 297 and 298; (794)
33) SEQ ID NOs: 54, 55, 24, 291, 292 and 293; (894)
34) SEQ ID NOs: 54, 55, 24, 291, 294 and 295; (795)
35) SEQ ID NOs: 54, 55, 24, 291, 297 and 298; (796)
36) SEQ ID NOs: 4, 30, 31, 291, 292 and 293; (884)
37) SEQ ID NOs: 4, 30, 31, 291, 294 and 295; (786)
38) SEQ ID NOs: 4, 30, 31, 291, 297 and 298; (787)
39) SEQ ID NOs: 4, 36, 29, 288, 289 and 290; (058)
40) SEQ ID NOs: 33, 34, 35, 296, 308 and 309; (284)
41) SEQ ID NOs: 33, 34, 35, 296, 308 and 309; (183)
42) SEQ ID NOs: 33, 34, 35, 296, 308 and 309; (184)
43) SEQ ID NOs: 19, 37, 38, 302, 303 and 304; (239)
44) SEQ ID NOs: 19, 37, 38, 296, 308 and 309; (286) or
45) SEQ ID NOs: 19, 51, 52, 302, 303 and 304; (247).

58-60. (canceled)

61. The bispecific antibody of claim 38, comprising a VHH1-CDR1, a VHH1-CDR2, and a VHH1-CDR3 and a VHH2-CDR1, a VHH2-CDR2, and a VHH2-CDR3, comprising the following amino acid sequences, respectively (E07-A05):
1) SEQ ID NOs: 22, 23, 24, 291, 292 and 293; (877; according to Kabat)
2) SEQ ID NOs: 22, 25, 26, 291, 294 and 295; (647; according to Kabat)
3) SEQ ID NOs: 22, 27, 24, 291, 297 and 298; (195; according to Kabat)
4) SEQ ID NOs: 77, 78, 79, 329, 330 and 331; (877; according to IMGT)
5) SEQ ID NOs: 77, 80, 81, 329, 332 and 333; (647; according to IMGT)
6) SEQ ID NOs: 77, 82, 79, 334, 335 and 336; (195; according to IMGT)
7) SEQ ID NOs: 127, 128, 129, 369, 370 and 371; (877; according to Chothia)
8) SEQ ID NOs: 127, 130, 131, 369, 372 and 373; (647; according to Chothia)
9) SEQ ID NOs: 127, 130, 131, 374, 375 and 376; (195; according to Chothia)
10) SEQ ID NOs: 174, 175, 176, 409, 410 and 411; (877; according to Honegger)
11) SEQ ID NOs: 174, 177, 178, 409, 412 and 413; (647; according to Honegger) or
12) SEQ ID NOs: 174, 179, 176, 414, 415 and 416; (195; according to Honegger).

62. The bispecific antibody of claim 38, comprising a VHH1-CDR1, a VHH1-CDR2, and a VHH1-CDR3 and a VHH2-CDR1, a VHH2-CDR2, and a VHH2-CDR3, comprising the following amino acid sequences, respectively (F11-C04):
1) SEQ ID NOs: 33, 34, 35, 296, 308 and 309; (284; according to Kabat)
2) SEQ ID NOs: 33, 34, 35, 296, 308 and 309; (183; according to Kabat)
3) SEQ ID NOs: 33, 34, 35, 296, 308 and 309; (184; according to Kabat)
4) SEQ ID NOs: 88, 89, 90, 326, 327 and 328; (284; according to IMGT)
5) SEQ ID NOs: 88, 89, 90, 326, 327 and 328; (183; according to IMGT)
6) SEQ ID NOs: 88, 89, 90, 326, 327 and 328; (184; according to IMGT)
7) SEQ ID NOs: 137, 138, 139, 386, 387 and 388; (284; according to Chothia)
8) SEQ ID NOs: 137, 138, 139, 386, 387 and 388; (183; according to Chothia)
9) SEQ ID NOs: 137, 138, 139, 386, 387 and 388; (184; according to Chothia)
10) SEQ ID NOs: 185, 186, 187, 426, 427 and 428; (284; according to Honegger)
11) SEQ ID NOs: 709, 186, 187, 426, 427 and 428; (183; according to Honegger) or
12) SEQ ID NOs: 709, 186, 187, 426, 427 and 428; (184; according to Honegger).

63. The bispecific antibody of claim 38, wherein the first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 212 and 448; [A06con-A05con]
2) SEQ ID NOs: 212 and 449; [A06con-A05con]
3) SEQ ID NOs: 212 and 460; [A06con-C04con]
4) SEQ ID NOs: 233 and 448; [E07con-A05con]
5) SEQ ID NOs: 234 and 449; [E07con-A05con]
6) SEQ ID NOs: 233 and 460; [E07con-C04con]
7) SEQ ID NOs: 234 and 460; [E07con-C04con]
8) SEQ ID NOs: 710 and 448; [C09con-A05con]
9) SEQ ID NOs: 710 and 449; [C09con-A05con]
10) SEQ ID NOs: 710 and 460; [C09con-C04con]
11) SEQ ID NOs: 230 and 460 [G22con-C04con] or
12) SEQ ID NOs: 249 and 460 [F11con-C04con].

64. The bispecific antibody of claim 38, wherein the first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 235 and 450; (877)
2) SEQ ID NOs: 237 and 451; (647)
3) SEQ ID NOs: 239 and 452; (195)
4) SEQ ID NOs: 235 and 446; (048)
5) SEQ ID NOs: 237 and 447; (049)
6) SEQ ID NOs: 239 and 447; (050)
7) SEQ ID NOs: 235 and 461; (274)
8) SEQ ID NOs: 237 and 462; (771)
9) SEQ ID NOs: 239 and 462; (772)
10) SEQ ID NOs: 241 and 446; (054)
11) SEQ ID NOs: 243 and 447; (755)
12) SEQ ID NOs: 241 and 450; (883)
13) SEQ ID NOs: 243 and 451; (784)
14) SEQ ID NOs: 243 and 452; (785)
15) SEQ ID NOs: 213 and 446; (046)
16) SEQ ID NOs: 215 and 447; (747)
17) SEQ ID NOs: 213 and 450; (875)
18) SEQ ID NOs: 215 and 451; (776)
19) SEQ ID NOs: 215 and 452; (777)
20) SEQ ID NOs: 213 and 461; (272)
21) SEQ ID NOs: 215 and 462; (773)
22) SEQ ID NOs: 224 and 446; (052)
23) SEQ ID NOs: 226 and 447; (753)
24) SEQ ID NOs: 224 and 461; (278)
25) SEQ ID NOs: 226 and 462; (769)
26) SEQ ID NOs: 227 and 446; (047)
27) SEQ ID NOs: 229 and 447; (748)
28) SEQ ID NOs: 227 and 450; (876)
29) SEQ ID NOs: 229 and 451; (778)
30) SEQ ID NOs: 229 and 452; (779)
31) SEQ ID NOs: 267 and 446; (062)
32) SEQ ID NOs: 269 and 447; (763)
33) SEQ ID NOs: 267 and 450; (891)
34) SEQ ID NOs: 269 and 451; (780)
35) SEQ ID NOs: 269 and 452; (781)
36) SEQ ID NOs: 267 and 461; (290)
37) SEQ ID NOs: 269 and 462; (791)
38) SEQ ID NOs: 270 and 450; (892)
39) SEQ ID NOs: 272 and 451; (793)
40) SEQ ID NOs: 272 and 452; (794)
41) SEQ ID NOs: 285 and 450; (894)
42) SEQ ID NOs: 287 and 451; (795)
43) SEQ ID NOs: 287 and 452; (796)
44) SEQ ID NOs: 244 and 450; (884)
45) SEQ ID NOs: 246 and 451; (786)
46) SEQ ID NOs: 247 and 452; (787)
47) SEQ ID NOs: 253 and 446; (058)
48) SEQ ID NOs: 255 and 447; (759)
49) SEQ ID NOs: 250 and 461; (284)
50) SEQ ID NOs: 251 and 462; (183)
51) SEQ ID NOs: 252 and 462; (184)
52) SEQ ID NOs: 256 and 456; (239)
53) SEQ ID NOs: 258 and 457; (740)
54) SEQ ID NOs: 256 and 461; (286)
55) SEQ ID NOs: 258 and 462; (788)
56) SEQ ID NOs: 278 and 456; (247) or
57) SEQ ID NOs: 280 and 457; (749).

65. The bispecific antibody of claim 38, wherein the first VHH that specifically binds to IL-10Rα and the second VHH that specifically binds to IL-10Rβ comprise the following amino acid sequences, respectively, or amino acid sequences that are at least 90%, at least 91%, at least 92%, at least 93%, at least 94%, at least 95%, at least 96%, at least 97%, at least 98%, or at least 99% identical, to the amino acid sequences set forth, respectively, in:
1) SEQ ID NOs: 236 and 450; (877)
2) SEQ ID NOs: 238 and 451; (647)
3) SEQ ID NOs: 240 and 452; (195)
4) SEQ ID NOs: 236 and 446; (048)
5) SEQ ID NOs: 238 and 447; (049)
6) SEQ ID NOs: 240 and 447; (050)
7) SEQ ID NOs: 236 and 461; (274)
8) SEQ ID NOs: 238 and 462; (771)
9) SEQ ID NOs: 240 and 462; (772)
10) SEQ ID NOs: 242 and 446; (054)
11) SEQ ID NOs: 243 and 447; (755)
12) SEQ ID NOs: 242 and 450; (883)
13) SEQ ID NOs: 243 and 451; (784)
14) SEQ ID NOs: 243 and 452; (785)
15) SEQ ID NOs: 214 and 446; (046)
16) SEQ ID NOs: 215 and 447; (747)
17) SEQ ID NOs: 214 and 450; (875)
18) SEQ ID NOs: 215 and 451; (776)
19) SEQ ID NOs: 215 and 452; (777)
20) SEQ ID NOs: 214 and 461; (272)
21) SEQ ID NOs: 215 and 462; (773)
22) SEQ ID NOs: 225 and 446; (052)
23) SEQ ID NOs: 226 and 447; (753)
24) SEQ ID NOs: 225 and 461; (278)
25) SEQ ID NOs: 226 and 462; (769)
26) SEQ ID NOs: 228 and 446; (047)
27) SEQ ID NOs: 229 and 447; (748)
28) SEQ ID NOs: 228 and 450; (876)
29) SEQ ID NOs: 229 and 451; (778)
30) SEQ ID NOs: 229 and 452; (779)
31) SEQ ID NOs: 268 and 446; (062)
32) SEQ ID NOs: 269 and 447; (763)
33) SEQ ID NOs: 268 and 450; (891)
34) SEQ ID NOs: 269 and 451; (780)
35) SEQ ID NOs: 269 and 452; (781)
36) SEQ ID NOs: 268 and 461; (290)
37) SEQ ID NOs: 269 and 462; (791)
38) SEQ ID NOs: 271 and 450; (892)
39) SEQ ID NOs: 273 and 451; (793)
40) SEQ ID NOs: 274 and 452; (794)
41) SEQ ID NOs: 286 and 450; (894)
42) SEQ ID NOs: 287 and 451; (795)
43) SEQ ID NOs: 287 and 452; (796)
44) SEQ ID NOs: 245 and 450; (884)
45) SEQ ID NOs: 246 and 451; (786)
46) SEQ ID NOs: 246 and 452; (787)
47) SEQ ID NOs: 254 and 446; (058)
48) SEQ ID NOs: 255 and 447; (759)
49) SEQ ID NOs: 250 and 461; (284)
50) SEQ ID NOs: 251 and 462; (183)
51) SEQ ID NOs: 252 and 462; (184)
52) SEQ ID NOs: 257 and 456; (239)
53) SEQ ID NOs: 259 and 457; (740)
54) SEQ ID NOs: 257 and 461; (286)
55) SEQ ID NOs: 259 and 462; (788)
56) SEQ ID NOs: 279 and 456; (247) or
57) SEQ ID NOs: 280 and 457; (749).

66-67. (canceled)

68. The bispecific antibody of claim 38, comprising in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 475-476 and 573.

69. The bispecific antibody of claim 38, comprising in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 574, 576, 578, 580, 582, 584, 586, 588 and 590.

70. The bispecific antibody of claim 38, comprising in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 575, 577, 579, 581, 583, 585, 587, 589, 591, 737-751 and 757-758.

71. The bispecific antibody of claim 38, comprising in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 475-482 and 737-751 (E07-A05).

72. The bispecific antibody of claim 38, comprising in tandem the first VHH that specifically binds to IL 10Rα and the second VHH that specifically binds to IL-10Rβ, comprising the following amino acid sequence, or an amino acid sequence that is at least 80%, at least 85%, at least 90%, at least 91%, at least 92%, at least 93%, at least 94%, at least 95%, at least 96%, at least 97%, at least 98%, or at least 99% identical, to the amino acid sequence selected from SEQ ID NOs: 573-579 and 757-758 (F11-C04).

73-88. (canceled)

89. Means for agonizing an IL-10 receptor that selectively suppresses activation of myeloid cells and induces minimal or no direct activation of T cells or B cells.

90-104. (canceled)

105. A polynucleotide that encodes the bispecific antibody of claim 38.

106-107. (canceled)

108. An expression cassette comprising one or more regulatory sequences operably linked to the polynucleotide of claim 105.

109-111. (canceled)

112. A cell or population of cells, comprising the polynucleotide of claim 105.

113-115. (canceled)

116. A method of producing an IL-10R binding molecule, the method comprising:
a) culturing a cell or population of cells of claim 112 in a cell culture under conditions sufficient to express the IL-10R binding molecule; and
b) isolating or purifying the antigen binding molecules from the cell culture.

117. A method of preventing or treating an inflammatory disease in a subject in need thereof, comprising administering to the subject an effective amount of a bispecific antibody of claim 38.

118. A method of reducing the severity of or mitigating on or more symptoms of an inflammatory disease in a subject in need thereof, comprising administering to the subject an effective amount of a bispecific antibody of claim 38.

119-137. (canceled)

138. A kit comprising one or more unitary doses of a bispecific antibody of claim 38.

139-145. (canceled)


SEQUENCE LISTING (794 sequences)

SEQ ID NO: 1 - protein, 5 aa, synthetic construct
NYWVD

SEQ ID NO: 2 - protein, 17 aa, synthetic construct
AINKDGGSAYYADSVKG

SEQ ID NO: 3 - protein, 8 aa, synthetic construct
DYGSGMHD

SEQ ID NO: 4 - protein, 5 aa, synthetic construct
SYGMG

SEQ ID NO: 5 - protein, 17 aa, synthetic construct
AINWIPGTTYYADSVKG

SEQ ID NO: 6 - protein, 15 aa, synthetic construct
RRRIDNYYRASEYDY

SEQ ID NO: 7 - protein, 5 aa, synthetic construct
SYAID

SEQ ID NO: 8 - protein, 17 aa, synthetic construct
CISSSGISTTYADSVKG

SEQ ID NO: 9 - protein, 15 aa, synthetic construct
AALCMSGTYYAGPGY

SEQ ID NO: 10 - protein, 5 aa, synthetic construct
YYAIG

SEQ ID NO: 11 - protein, 17 aa, synthetic construct
CISSSGGSTYYADSVKG

SEQ ID NO: 12 - protein, 17 aa, synthetic construct
DAACMVVAGTLILPDDY

SEQ ID NO: 13 - protein, 17 aa, synthetic construct
TINWIPGSTYYADSVKG

SEQ ID NO: 14 - protein, 15 aa, synthetic construct
RRRIESYYRASEYDY

SEQ ID NO: 15 - protein, 17 aa, synthetic construct
AISWIGGGTYYADSVKG

SEQ ID NO: 16 - protein, 15 aa, synthetic construct
KYYPDSYFAAASYAY

SEQ ID NO: 17 - protein, 17 aa, synthetic construct
AISWIGGSTYYADSVKG

SEQ ID NO: 18 - protein, 15 aa, synthetic construct
KYYTDSYFAATSYAY

SEQ ID NO: 19 - protein, 5 aa, synthetic construct
GYGMG

SEQ ID NO: 20 - protein, 17 aa, synthetic construct
SINWIYGATYYTDSVKG

SEQ ID NO: 21 - protein, 15 aa, synthetic construct
KINGGSYFNTGSFGF

SEQ ID NO: 22 - protein, 5 aa, synthetic construct
AYGMG

SEQ ID NO: 23 - protein, 17 aa, synthetic construct
AIRWIGDSTYYADSVKG

SEQ ID NO: 24 - protein, 15 aa, synthetic construct
KTSVTSYLSEKSYNY

SEQ ID NO: 25 - protein, 17 aa, synthetic construct
AIRWYGEGTYYAESVKG

SEQ ID NO: 26 - protein, 15 aa, synthetic construct
KTRPDSYLSEKSYGY

SEQ ID NO: 27 - protein, 17 aa, synthetic construct
AIRWIGDATYYAESVKG

SEQ ID NO: 28 - protein, 17 aa, synthetic construct
AISWIGNSAYYADSVKG

SEQ ID NO: 29 - protein, 15 aa, synthetic construct
KYYTDSYYAAASYAY

SEQ ID NO: 30 - protein, 17 aa, synthetic construct
AISWIGSATDYADSVRG

SEQ ID NO: 31 - protein, 15 aa, synthetic construct
RRSPMSRTSVLQYDY

SEQ ID NO: 32 - protein, 15 aa, synthetic construct
KTSVTSYLSEKAYNY

SEQ ID NO: 33 - protein, 5 aa, synthetic construct
SHTVG

SEQ ID NO: 34 - protein, 17 aa, synthetic construct
AITWNGDTTYFGDFVEG

SEQ ID NO: 35 - protein, 18 aa, synthetic construct
ATGQVWSLQYLRESSYAY

SEQ ID NO: 36 - protein, 17 aa, synthetic construct
AISWIGNSTYYADSVKG

SEQ ID NO: 37 - protein, 17 aa, synthetic construct
AINWIYGATYYADSVKG

SEQ ID NO: 38 - protein, 15 aa, synthetic construct
KINAGSYFNIGSFGS

SEQ ID NO: 39 - protein, 5 aa, synthetic construct
DDTVG

SEQ ID NO: 40 - protein, 17 aa, synthetic construct
AITWNGDTTYYGDSVKG

SEQ ID NO: 41 - protein, 18 aa, synthetic construct
AAGQDWSLGYLRESSYAY

SEQ ID NO: 42 - protein, 5 aa, synthetic construct
GYTVG

SEQ ID NO: 43 - protein, 17 aa, synthetic construct
AITWNGDTTYFGDSVKG

SEQ ID NO: 44 - protein, 18 aa, synthetic construct
ATGQEWSLQYLRESSYAY

SEQ ID NO: 45 - protein, 5 aa, synthetic construct
NYAMA

SEQ ID NO: 46 - protein, 17 aa, synthetic construct
AINWFGRATEYADSVKD

SEQ ID NO: 47 - protein, 16 aa, synthetic construct
RTKVTTGRRNQAEYDY

SEQ ID NO: 48 - protein, 17 aa, synthetic construct
TINWIPGNTYYADSVKG

SEQ ID NO: 49 - protein, 18 aa, synthetic construct
TIIGWIPGNTYYADSVKG

SEQ ID NO: 50 - protein, 15 aa, synthetic construct
RRRIDSYYRASEYDY

SEQ ID NO: 51 - protein, 17 aa, synthetic construct
SINWIYGATYYSDSVKG

SEQ ID NO: 52 - protein, 15 aa, synthetic construct
KINGESYFNTGSFGS

SEQ ID NO: 53 - protein, 15 aa, synthetic construct
KINGGSYFNIGSFGS

SEQ ID NO: 54 - protein, 5 aa, synthetic construct
YYGMG

SEQ ID NO: 55 - protein, 17 aa, synthetic construct
AIRWIGGDTYYADSVKG

SEQ ID NO: 56 - protein, 8 aa, synthetic construct
GFTFSNYW

SEQ ID NO: 57 - protein, 8 aa, synthetic construct
INKDGGSA

SEQ ID NO: 58 - protein, 10 aa, synthetic construct
AKDYGSGMHD

SEQ ID NO: 59 - protein, 8 aa, synthetic construct
GRTFSSYG

SEQ ID NO: 60 - protein, 8 aa, synthetic construct
INWIPGTT

SEQ ID NO: 61 - protein, 17 aa, synthetic construct
AARRRIDNYYRASEYDY

SEQ ID NO: 62 - protein, 8 aa, synthetic construct
GFTLDSYA

SEQ ID NO: 63 - protein, 8 aa, synthetic construct
ISSSGIST

SEQ ID NO: 64 - protein, 17 aa, synthetic construct
AAAALCMSGTYYAGPGY

SEQ ID NO: 65 - protein, 8 aa, synthetic construct
DFTLDYYA

SEQ ID NO: 66 - protein, 8 aa, synthetic construct
ISSSGGST

SEQ ID NO: 67 - protein, 19 aa, synthetic construct
AADAACMVVAGTLILPDDY

SEQ ID NO: 68 - protein, 8 aa, synthetic construct
INWIPGST

SEQ ID NO: 69 - protein, 17 aa, synthetic construct
AARRRIESYYRASEYDY

SEQ ID NO: 70 - protein, 8 aa, synthetic construct
ISWIGGGT

SEQ ID NO: 71 - protein, 17 aa, synthetic construct
AAKYYPDSYFAAASYAY

SEQ ID NO: 72 - protein, 8 aa, synthetic construct
ISWIGGST

SEQ ID NO: 73 - protein, 17 aa, synthetic construct
AAKYYTDSYFAATSYAY

SEQ ID NO: 74 - protein, 8 aa, synthetic construct
GRTFSGYG

SEQ ID NO: 75 - protein, 8 aa, synthetic construct
INWIYGAT

SEQ ID NO: 76 - protein, 17 aa, synthetic construct
AAKINGGSYFNTGSFGF

SEQ ID NO: 77 - protein, 8 aa, synthetic construct
GRTFSAYG

SEQ ID NO: 78 - protein, 8 aa, synthetic construct
IRWIGDST

SEQ ID NO: 79 - protein, 17 aa, synthetic construct
AAKTSVTSYLSEKSYNY

SEQ ID NO: 80 - protein, 8 aa, synthetic construct
IRWYGEGT

SEQ ID NO: 81 - protein, 17 aa, synthetic construct
AAKTRPDSYLSEKSYGY

SEQ ID NO: 82 - protein, 8 aa, synthetic construct
IRWIGDAT

SEQ ID NO: 83 - protein, 8 aa, synthetic construct
ISWIGNSA

SEQ ID NO: 84 - protein, 17 aa, synthetic construct
AAKYYTDSYYAAASYAY

SEQ ID NO: 85 - protein, 8 aa, synthetic construct
ISWIGSAT

SEQ ID NO: 86 - protein, 17 aa, synthetic construct
AARRSPMSRTSVLQYDY

SEQ ID NO: 87 - protein, 17 aa, synthetic construct
AAKTSVTSYLSEKAYNY

SEQ ID NO: 88 - protein, 8 aa, synthetic construct
GRTFSSHT

SEQ ID NO: 89 - protein, 8 aa, synthetic construct
ITWNGDTT

SEQ ID NO: 90 - protein, 20 aa, synthetic construct
AAATGQVWSLQYLRESSYAY

SEQ ID NO: 91 - protein, 8 aa, synthetic construct
ISWIGNST

SEQ ID NO: 92 - protein, 8 aa, synthetic construct
GRTFDGYG

SEQ ID NO: 93 - protein, 17 aa, synthetic construct
AAKINAGSYFNIGSFGS

SEQ ID NO: 94 - protein, 8 aa, synthetic construct
GRTFSDDT

SEQ ID NO: 95 - protein, 20 aa, synthetic construct
AAAAGQDWSLGYLRESSYAY

SEQ ID NO: 96 - protein, 8 aa, synthetic construct
GRTFSGYT

SEQ ID NO: 97 - protein, 20 aa, synthetic construct
AAATGQEWSLQYLRESSYAY

SEQ ID NO: 98 - protein, 8 aa, synthetic construct
GRTFSNYA

SEQ ID NO: 99 - protein, 8 aa, synthetic construct
INWFGRAT

SEQ ID NO: 100 - protein, 18 aa, synthetic construct
AARTKVTTGRRNQAEYDY

SEQ ID NO: 101 - protein, 8 aa, synthetic construct
INWIPGNT

SEQ ID NO: 102 - protein, 9 aa, synthetic construct
IIGWIPGNT

SEQ ID NO: 103 - protein, 17 aa, synthetic construct
AARRRIDSYYRASEYDY

SEQ ID NO: 104 - protein, 17 aa, synthetic construct
AAKINGESYFNTGSFGS

SEQ ID NO: 105 - protein, 17 aa, synthetic construct
AAKINGGSYFNIGSFGS

SEQ ID NO: 106 - protein, 8 aa, synthetic construct
GGTFSYYG

SEQ ID NO: 107 - protein, 8 aa, synthetic construct
IRWIGGDT

SEQ ID NO: 108 - protein, 7 aa, synthetic construct
GFTFSNY

SEQ ID NO: 109 - protein, 4 aa, synthetic construct
KDGG

SEQ ID NO: 110 - protein, 6 aa, synthetic construct
YGSGMH

SEQ ID NO: 111 - protein, 7 aa, synthetic construct
GRTFSSY

SEQ ID NO: 112 - protein, 4 aa, synthetic construct
WIPG

SEQ ID NO: 113 - protein, 13 aa, synthetic construct
RRIDNYYRASEYD

SEQ ID NO: 114 - protein, 7 aa, synthetic construct
GFTLDSY

SEQ ID NO: 115 - protein, 4 aa, synthetic construct
SSGI

SEQ ID NO: 116 - protein, 13 aa, synthetic construct
ALCMSGTYYAGPG

SEQ ID NO: 117 - protein, 7 aa, synthetic construct
DFTLDYY

SEQ ID NO: 118 - protein, 4 aa, synthetic construct
SSGG

SEQ ID NO: 119 - protein, 15 aa, synthetic construct
AACMVVAGTLILPDD

SEQ ID NO: 120 - protein, 13 aa, synthetic construct
RRIESYYRASEYD

SEQ ID NO: 121 - protein, 4 aa, synthetic construct
WIGG

SEQ ID NO: 122 - protein, 13 aa, synthetic construct
YYPDSYFAAASYA

SEQ ID NO: 123 - protein, 13 aa, synthetic construct
YYTDSYFAATSYA

SEQ ID NO: 124 - protein, 7 aa, synthetic construct
GRTFSGY

SEQ ID NO: 125 - protein, 4 aa, synthetic construct
WIYG

SEQ ID NO: 126 - protein, 13 aa, synthetic construct
INGGSYFNTGSFG

SEQ ID NO: 127 - protein, 7 aa, synthetic construct
GRTFSAY

SEQ ID NO: 128 - protein, 4 aa, synthetic construct
WIGD

SEQ ID NO: 129 - protein, 13 aa, synthetic construct
TSVTSYLSEKSYN

SEQ ID NO: 130 - protein, 4 aa, synthetic construct
WYGE

SEQ ID NO: 131 - protein, 13 aa, synthetic construct
TRPDSYLSEKSYG

SEQ ID NO: 132 - protein, 4 aa, synthetic construct
WIGN

SEQ ID NO: 133 - protein, 13 aa, synthetic construct
YYTDSYYAAASYA

SEQ ID NO: 134 - protein, 4 aa, synthetic construct
WIGS

SEQ ID NO: 135 - protein, 13 aa, synthetic construct
RSPMSRTSVLQYD

SEQ ID NO: 136 - protein, 13 aa, synthetic construct
TSVTSYLSEKAYN

SEQ ID NO: 137 - protein, 7 aa, synthetic construct
GRTFSSH

SEQ ID NO: 138 - protein, 4 aa, synthetic construct
WNGD

SEQ ID NO: 139 - protein, 16 aa, synthetic construct
TGQVWSLQYLRESSYA

SEQ ID NO: 140 - protein, 7 aa, synthetic construct
GRTFDGY

SEQ ID NO: 141 - protein, 13 aa, synthetic construct
INAGSYFNIGSFG

SEQ ID NO: 142 - protein, 7 aa, synthetic construct
GRTFSDD

SEQ ID NO: 143 - protein, 16 aa, synthetic construct
AGQDWSLGYLRESSYA

SEQ ID NO: 144 - protein, 16 aa, synthetic construct
TGQEWSLQYLRESSYA

SEQ ID NO: 145 - protein, 7 aa, synthetic construct
GRTFSNY

SEQ ID NO: 146 - protein, 4 aa, synthetic construct
WFGR

SEQ ID NO: 147 - protein, 14 aa, synthetic construct
TKVTTGRRNQAEYD

SEQ ID NO: 148 - protein, 5 aa, synthetic construct
GWIPG

SEQ ID NO: 149 - protein, 13 aa, synthetic construct
RRIDSYYRASEYD

SEQ ID NO: 150 - protein, 13 aa, synthetic construct
INGESYFNTGSFG

SEQ ID NO: 151 - protein, 13 aa, synthetic construct
INGGSYFNIGSFG

SEQ ID NO: 152 - protein, 7 aa, synthetic construct
GGTFSYY

SEQ ID NO: 153 - protein, 10 aa, synthetic construct
ASGFTFSNYW

SEQ ID NO: 154 - protein, 17 aa, synthetic construct
INKDGGSAYYADSVKGR

SEQ ID NO: 155 - protein, 7 aa, synthetic construct
DYGSGMH

SEQ ID NO: 156 - protein, 10 aa, synthetic construct
ASGRTFSSYG

SEQ ID NO: 157 - protein, 17 aa, synthetic construct
INWIPGTTYYADSVKGR

SEQ ID NO: 158 - protein, 14 aa, synthetic construct
RRRIDNYYRASEYD

SEQ ID NO: 159 - protein, 10 aa, synthetic construct
ASGFTLDSYA

SEQ ID NO: 160 - protein, 17 aa, synthetic construct
ISSSGISTTYADSVKGR

SEQ ID NO: 161 - protein, 14 aa, synthetic construct
AALCMSGTYYAGPG

SEQ ID NO: 162 - protein, 10 aa, synthetic construct
ASDFTLDYYA

SEQ ID NO: 163 - protein, 17 aa, synthetic construct
ISSSGGSTYYADSVKGR

SEQ ID NO: 164 - protein, 16 aa, synthetic construct
DAACMVVAGTLILPDD

SEQ ID NO: 165 - protein, 17 aa, synthetic construct
INWIPGSTYYADSVKGR

SEQ ID NO: 166 - protein, 14 aa, synthetic construct
RRRIESYYRASEYD

SEQ ID NO: 167 - protein, 17 aa, synthetic construct
ISWIGGGTYYADSVKGR

SEQ ID NO: 168 - protein, 14 aa, synthetic construct
KYYPDSYFAAASYA

SEQ ID NO: 169 - protein, 17 aa, synthetic construct
ISWIGGSTYYADSVKGR

SEQ ID NO: 170 - protein, 14 aa, synthetic construct
KYYTDSYFAATSYA

SEQ ID NO: 171 - protein, 10 aa, synthetic construct
ASGRTFSGYG

SEQ ID NO: 172 - protein, 17 aa, synthetic construct
INWIYGATYYTDSVKGR

SEQ ID NO: 173 - protein, 14 aa, synthetic construct
KINGGSYFNTGSFG

SEQ ID NO: 174 - protein, 10 aa, synthetic construct
ASGRTFSAYG

SEQ ID NO: 175 - protein, 17 aa, synthetic construct
IRWIGDSTYYADSVKGR

SEQ ID NO: 176 - protein, 14 aa, synthetic construct
KTSVTSYLSEKSYN

SEQ ID NO: 177 - protein, 17 aa, synthetic construct
IRWYGEGTYYAESVKGR

SEQ ID NO: 178 - protein, 14 aa, synthetic construct
KTRPDSYLSEKSYG

SEQ ID NO: 179 - protein, 17 aa, synthetic construct
IRWIGDATYYAESVKGR

SEQ ID NO: 180 - protein, 17 aa, synthetic construct
ISWIGNSAYYADSVKGR

SEQ ID NO: 181 - protein, 14 aa, synthetic construct
KYYTDSYYAAASYA

SEQ ID NO: 182 - protein, 17 aa, synthetic construct
ISWIGSATDYADSVRGR

SEQ ID NO: 183 - protein, 14 aa, synthetic construct
RRSPMSRTSVLQYD

SEQ ID NO: 184 - protein, 14 aa, synthetic construct
KTSVTSYLSEKAYN

SEQ ID NO: 185 - protein, 10 aa, synthetic construct
NSGRTFSSHT

SEQ ID NO: 186 - protein, 17 aa, synthetic construct
ITWNGDTTYFGDFVEGR

SEQ ID NO: 187 - protein, 17 aa, synthetic construct
ATGQVWSLQYLRESSYA

SEQ ID NO: 188 - protein, 17 aa, synthetic construct
ISWIGNSTYYADSVKGR

SEQ ID NO: 189 - protein, 10 aa, synthetic construct
ASGRTFDGYG

SEQ ID NO: 190 - protein, 17 aa, synthetic construct
INWIYGATYYADSVKGR

SEQ ID NO: 191 - protein, 14 aa, synthetic construct
KINAGSYFNIGSFG

SEQ ID NO: 192 - protein, 10 aa, synthetic construct
ASGRTFSDDT

SEQ ID NO: 193 - protein, 17 aa, synthetic construct
ITWNGDTTYYGDSVKGR

SEQ ID NO: 194 - protein, 17 aa, synthetic construct
AAGQDWSLGYLRESSYA

SEQ ID NO: 195 - protein, 10 aa, synthetic construct
ASGRTFSGYT

SEQ ID NO: 196 - protein, 17 aa, synthetic construct
ITWNGDTTYFGDSVKGR

SEQ ID NO: 197 - protein, 17 aa, synthetic construct
ATGQEWSLQYLRESSYA

SEQ ID NO: 198 - protein, 10 aa, synthetic construct
ASGRTFSNYA

SEQ ID NO: 199 - protein, 17 aa, synthetic construct
INWFGRATEYADSVKDR

SEQ ID NO: 200 - protein, 15 aa, synthetic construct
RTKVTTGRRNQAEYD

SEQ ID NO: 201 - protein, 17 aa, synthetic construct
INWIPGNTYYADSVKGR

SEQ ID NO: 202 - protein, 18 aa, synthetic construct
IIGWIPGNTYYADSVKGR

SEQ ID NO: 203 - protein, 14 aa, synthetic construct
RRRIDSYYRASEYD

SEQ ID NO: 204 - protein, 10 aa, synthetic construct
NSGRTFSGYG

SEQ ID NO: 205 - protein, 17 aa, synthetic construct
INWIYGATYYSDSVKGR

SEQ ID NO: 206 - protein, 14 aa, synthetic construct
KINGESYFNTGSFG

SEQ ID NO: 207 - protein, 14 aa, synthetic construct
KINGGSYFNIGSFG

SEQ ID NO: 208 - protein, 10 aa, synthetic construct
ASGGTFSYYG

SEQ ID NO: 209 - protein, 17 aa, synthetic construct
IRWIGGDTYYADSVKGR

SEQ ID NO: 210 - protein, 117 aa, synthetic construct
DVQLVESGGGVVQPGGSLRLSCAASGFTFSNYWVDWLRQAPGKGLEWVSAINKDGGSAYYADSVKGRFTISRDNAKNTLYLQMTSLKPEDTAVYYCAKDYGSGMHDWGKGTQVTVSS

SEQ ID NO: 211 - protein, 117 aa, synthetic construct
EVQLVESGGGLVQPGGSLRLSCAASGFTFSNYWVDWLRQAPGKGLEWVSAINKDGGSAYYADSVKGRFTISRDNSKNTLYLQMNSLRAEDTAVYYCAKDYGSGMHDWGQGTMVTVSS

SEQ ID NO: 212 - protein, 128 aa, synthetic construct
  VARIANT 1..3: note = S, GG, AS, ASS or absent
  VARIANT 4: note = E or Q
  VARIANT 14: note = L, S or V
  VARIANT 17: note = A or P
  VARIANT 49: note = E or G
  VARIANT 50: note = F or L
  VARIANT 53: note = A or T
  VARIANT 55: note = I or absent
  VARIANT 61: note = N, S or T
  VARIANT 75: note = S or T
  VARIANT 89: note = N or S
  VARIANT 91: note = K or R
  VARIANT 79: note = D or S
  VARIANT 92: note = A or P
  VARIANT 107: note = D or E
  VARIANT 108: note = N or S
  VARIANT 123: note = L or Q
XXXXLQLVESGGGXVQXGGSLRLSCAASGRTFSSYGMGWFRQAPGKERXXVAXIXNWIPGXTYYADSVKGRFTIXGDKXKNTLYLQMDXLXXEDTAVYYCAARRRIXXYYRASEYDYWGQGTXVTVSS

SEQ ID NO: 213 - protein, 127 aa, synthetic construct
  VARIANT 1..3: note = S, GG, AS, ASS or absent
XXXQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSS

SEQ ID NO: 214 - protein, 126 aa, synthetic construct
ASQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSS

SEQ ID NO: 215 - protein, 124 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSS

SEQ ID NO: 216 - protein, 124 aa, synthetic construct
DLQLVESGGGLVQPGGSLRLSCAASGFTLDSYAIDWFRQAPGKEREGVSCISSSGISTTYADSVKGRFTISRDNAKNTVYLQMNSLKPEDTAVYYCAAAALCMSGTYYAGPGYWGQGTQVTVSS

SEQ ID NO: 217 - protein, 124 aa, synthetic construct
ELQLVESGGGLVQPGGSLRLSCAASGFTLDSYAIDWFRQAPGKEREGVSCISSSGISTTYADSVKGRFTISRDNSKNTVYLQMNSLRAEDTAVYYCAAAALCMSGTYYAGPGYWGQGTTVTVSS

SEQ ID NO: 218 - protein, 126 aa, synthetic construct
DVQLVESGGGLVQPGGSLRLSCAASDFTLDYYAIGWFRQAPGKEREGVSCISSSGGSTYYADSVKGRFTISRDNAKNTVSLQMNSLKPEDTAVYYCAADAACMVVAGTLILPDDYWGQGTQVTVSS

SEQ ID NO: 219 - protein, 126 aa, synthetic construct
EVQLVESGGGLVQPGGSLRLSCAASGFTLDYYAIGWFRQAPGKEREGVSCISSSGGSTYYADSVKGRFTISRDNSKNTVYLQMNSLRAEDTAVYYCAADAACMVVAGTLILPDDYWGQGTLVTVSS

SEQ ID NO: 220 - protein, 127 aa, synthetic construct
  VARIANT 1..3: note = S, GG, AS, ASS or absent
XXXQVQLVESGGGSVQAGGSLRLSCAASGRTFSSYGMGWFRQAPGKERGFVATINWIPGSTYYADSVKGRFTISGDKDNNTVYLQMDNLKPEDTAVYYCAARRRIESYYRASEYDYWGQGTQVTVSS

SEQ ID NO: 221 - protein, 126 aa, synthetic construct
ASQVQLVESGGGSVQAGGSLRLSCAASGRTFSSYGMGWFRQAPGKERGFVATINWIPGSTYYADSVKGRFTISGDKDNNTVYLQMDNLKPEDTAVYYCAARRRIESYYRASEYDYWGQGTQVTVSS

SEQ ID NO: 222 - protein, 127 aa, synthetic construct
  VARIANT 1..3: note = S, GG, AS, ASS or absent
XXXQVQLVESGGGVVQPGGSLRLSCAASGRTFSSYGMGWFRQAPGKEREFVATINWIPGSTYYADSVKGRFTISGDKSKNTVYLQMNSLRAEDTAVYYCAARRRIESYYRASEYDYWGQGTLVTVSS

SEQ ID NO: 223 - protein, 126 aa, synthetic construct
ASQVQLVESGGGVVQPGGSLRLSCAASGRTFSSYGMGWFRQAPGKEREFVATINWIPGSTYYADSVKGRFTISGDKSKNTVYLQMNSLRAEDTAVYYCAARRRIESYYRASEYDYWGQGTLVTVSS

SEQ ID NO: 224 - protein, 127 aa, synthetic construct
  VARIANT 1..3: note = S, GG, AS, ASS or absent
XXXQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSS

SEQ ID NO: 225 - protein, 126 aa, synthetic construct
ASQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSS

SEQ ID NO: 226 - protein, 124 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSS

SEQ ID NO: 227 - protein, 127 aa, synthetic construct
  VARIANT 1..3: note = S, GG, AS, ASS or absent
XXXQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSS

SEQ ID NO: 228 - protein, 126 aa, synthetic construct
ASQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSS

SEQ ID NO: 229 - protein, 124 aa, synthetic construct; identical to SEQ ID NO: 226

SEQ ID NO: 230 - protein, 127 aa, synthetic construct
  VARIANT 1..3: note = S, GG, AS, ASS or absent
  VARIANT 4: note = E or Q
  VARIANT 8: note = L, Q or V
  VARIANT 17: note = A or P
  VARIANT 22: note = K or R
  VARIANT 26: note = A, S or T
  VARIANT 27: note = A or N
  VARIANT 43: note = A or P
  VARIANT 53: note = A or S
  VARIANT 64: note = A, S or T
  VARIANT 72: note = A or T
  VARIANT 73: note = L or V
  VARIANT 75: note = G or R
  VARIANT 78: note = A, D or S
  VARIANT 80: note = G or N
  VARIANT 82: note = L, M or V
  VARIANT 87: note = D, N or S
  VARIANT 90: note = K or R
  VARIANT 91: note = A or P
  VARIANT 106: note = E or G
  VARIANT 111: note = I or T
  VARIANT 122: note = L or Q
XXXXLQLXESGGGLVQXGGSLXLSCXXSGRTFSGYGMGWFRQXPGKEREFVSXINWIYGATYYXDSVKGRFXXSXDKXKXTXYLQMXSLXXEDTAVYYCAAKINGXSYFNXGSFGFWGQGTXVTVSS

SEQ ID NO: 231 - protein, 124 aa, synthetic construct
ELQLVESGGGLVQAGGSLKLSCTASGRTFSGYGMGWFRQAPGKEREFVSSINWIYGATYYTDSVKGRFAVSRDKDKNTMYLQMSSLKPEDTAVYYCAAKINGGSYFNTGSFGFWGQGTQVTVSS

SEQ ID NO: 232 - protein, 124 aa, synthetic construct
ELQLVESGGGLVQPGGSLRLSCAASGRTFSGYGMGWFRQAPGKEREFVSSINWIYGATYYTDSVKGRFTVSRDKSKNTMYLQMNSLRAEDTAVYYCAAKINGGSYFNTGSFGFWGQGTLVTVSS

SEQ ID NO: 233 - protein, 127 aa, synthetic construct
  VARIANT 1..3: note = S, GG, AS, ASS or absent
  VARIANT 4: note = E or Q
  VARIANT 16: note = Q or R
  VARIANT 17: note = A or P
  VARIANT 19: note = D or G
  VARIANT 47: note = E or G
  VARIANT 48: note = L or R
  VARIANT 52: note = A or S
  VARIANT 57: note = I or Y
  VARIANT 59: note = D or E
  VARIANT 60: note = A, G or S
  VARIANT 65: note = D or E
  VARIANT 77: note = N or S
  VARIANT 78: note = A or S
  VARIANT 87: note = N or S
  VARIANT 90: note = K or R
  VARIANT 91: note = A or P
  VARIANT 104: note = R or S
  VARIANT 105: note = P or V
  VARIANT 106: note = D or T
  VARIANT 115: note = G or N
  VARIANT 122: note = M or Q
XXXXLQLVESGGGLVXXGXSLRLSCAASGRTFSAYGMGWFRQAPGKXXEFVXAIRWXGXXTYYAXSVKGRFTISKDXXKNTLYLQMXSLXXEDTAVYYCAAKTXXXSYLSEKSYXYWGQGTXVTVSS

SEQ ID NO: 234 - protein, 127 aa, synthetic construct
  VARIANT 1..3: note = S, GG, AS, ASS or absent
  VARIANT 16: note = Q or R
  VARIANT 17: note = A or P
  VARIANT 47: note = E or G
  VARIANT 52: note = A or S
  VARIANT 57: note = I or Y
  VARIANT 59: note = D or E
  VARIANT 60: note = A, G or S
  VARIANT 65: note = D or E
  VARIANT 78: note = A or S
  VARIANT 87: note = N or S
  VARIANT 90: note = K or R
  VARIANT 91: note = A or P
  VARIANT 104: note = R or S
  VARIANT 105: note = P or V
  VARIANT 106: note = D or T
  VARIANT 115: note = G or N
  VARIANT 122: note = M or Q
XXXQLQLVESGGGLVXXGGSLRLSCAASGRTFSAYGMGWFRQAPGKXREFVXAIRWXGXXTYYAXSVKGRFTISKDNXKNTLYLQMXSLXXEDTAVYYCAAKTXXXSYLSEKSYXYWGQGTXVTVSS

SEQ ID NO: 235 - protein, 127 aa, synthetic construct
  VARIANT 1..3: note = S, GG, AS, ASS or absent
XXXQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSS

SEQ ID NO: 236 - protein, 126 aa, synthetic construct
ASQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSS

SEQ ID NO: 237 - protein, 127 aa, synthetic construct
  VARIANT 1..3: note = S, GG, AS, ASS or absent
XXXQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSS

SEQ ID NO: 238 - protein, 126 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSS

SEQ ID NO: 239 - protein, 127 aa, synthetic construct
  VARIANT 1..3: note = S, GG, AS, ASS or absent
XXXQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSS

SEQ ID NO: 240 - protein, 126 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSS

SEQ ID NO: 241 - protein, 127 aa, synthetic construct
  VARIANT 1..3: note = S, GG, AS, ASS or absent
XXXQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSS

SEQ ID NO: 242 - protein, 126 aa, synthetic construct
ASQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSS

SEQ ID NO: 243 - protein, 124 aa, synthetic construct
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSS

SEQ ID NO: 244 - protein, 127 aa, synthetic construct
  VARIANT 1..3: note = S, GG, AS, ASS or absent
XXXQVQLQESGGGLVQAGGSLSLSCAASGRTFSSYGMGWFRQAPGKEREFVAAISWIGSATDYADSVRGRFTISGDNAKNTVYLLMNNLKPEDTAVYYCAARRSPMSRTSVLQYDYWGQGTQVTVSS

SEQ ID NO: 245 - protein, 126 aa, synthetic construct
ASQVQLQESGGGLVQAGGSLSLSCAASGRTFSSYGMGWFRQAPGKEREFVAAISWIGSATDYADSVRGRFTISGDNAKNTVYLLMNNLKPEDTAVYYCAARRSPMSRTSVLQYDYWGQGTQVTVSS

SEQ ID NO: 246 - protein, 124 aa, synthetic construct
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSS

SEQ ID NO: 247 - protein, 124 aa, synthetic construct
EVQLVESGGGLVQAGDSLRLSCAASGRTFSAYGMGWFRQAPGKEREFVAAIRWIGDSTYYADSVKGRFTISKDSAKNTLYLQMSSLKPEDTAVYYCAAKTSVTSYLSEKAYNYWGQGTQVTVSS

SEQ ID NO: 248 - protein, 124 aa, synthetic construct
EVQLVESGGGLVQPGGSLRLSCAASGRTFSAYGMGWFRQAPGKEREFVAAIRWIGDSTYYADSVKGRFTISKDSSKNTLYLQMNSLRAEDTAVYYCAAKTSVTSYLSEKAYNYWGQGTMVTVSS

SEQ ID NO: 249 - protein, 127 aa, synthetic construct
  VARIANT 2: note = L or V
  VARIANT 5: note = L or V
  VARIANT 14: note = A or P
  VARIANT 24: note = A or N
  VARIANT 44: note = E or G
  VARIANT 49: note = A or E
  VARIANT 69: note = S or T
  VARIANT 74: note = N or S
  VARIANT 75: note = A or S
  VARIANT 82: note = E or Q
  VARIANT 85: note = K or S
  VARIANT 87: note = K or R
  VARIANT 88: note = A or P
  VARIANT 122: note = L or Q
EXQLXESGGGLVQXGGSLRLSCAXSGRTFSSHTVGWFRQAPGKXREFVXAITWNGDTTYFGDFVEGRFXISRDXXKNTLYLXMNXLXXEDTAVYYCAAATGQVWSLQYLRESSYAYWGQGTXVTVSS

SEQ ID NO: 250 - protein, 127 aa, synthetic construct
ELQLVESGGGLVQAGGSLRLSCANSGRTFSSHTVGWFRQAPGKEREFVAAITWNGDTTYFGDFVEGRFSISRDSAKNTLYLEMNKLKPEDTAVYYCAAATGQVWSLQYLRESSYAYWGQGTQVTVSS

SEQ ID NO: 251 - protein, 127 aa, synthetic construct
EVQLLESGGGLVQPGGSLRLSCAASGRTFSSHTVGWFRQAPGKGREFVAAITWNGDTTYFGDFVEGRFTISRDNSKNTLYLQMNSLRAEDTAVYYCAAATGQVWSLQYLRESSYAYWGQGTLVTVSS

SEQ ID NO: 252 - protein, 127 aa, synthetic construct
ELQLLESGGGLVQPGGSLRLSCAASGRTFSSHTVGWFRQAPGKGREFVSAITWNGDTTYFGDFVEGRFTISRDNSKNTLYLQMNSLRAEDTAVYYCAAATGQVWSLQYLRESSYAYWGQGTLVTVSS

SEQ ID NO: 253 - protein, 127 aa, synthetic construct
  VARIANT 1..3: note = S, GG, AS, ASS or absent
XXXQVQLVESGGGLVQAGGSLRLSCAASGRTFSSYGMGWFRQAPGKEREFVAAISWIGNSTYYADSVKGRFTLSGDNAKNTLYLQMNSLKPEDTAVYYCAAKYYTDSYYAAASYAYWGQGTQVTVSS

SEQ ID NO: 254 - protein, 126 aa, synthetic construct
ASQVQLVESGGGLVQAGGSLRLSCAASGRTFSSYGMGWFRQAPGKEREFVAAISWIGNSTYYADSVKGRFTLSGDNAKNTLYLQMNSLKPEDTAVYYCAAKYYTDSYYAAASYAYWGQGTQVTVSS

SEQ ID NO: 255 - protein, 124 aa, synthetic construct
EVQLVESGGGLVQPGGSLRLSCAASGRTFSSYGMGWFRQAPGKEREFVAAISWIGNSTYYADSVKGRFTLSGDNSKNTLYLQMNSLRAEDTAVYYCAAKYYTDSYYAAASYAYWGQGTLVTVSS

SEQ ID NO: 256 - protein, 127 aa, synthetic construct
  VARIANT 1..3: note = S, GG, AS, ASS or absent
XXXQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSS

SEQ ID NO: 257 - protein, 126 aa, synthetic construct
ASQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSS

SEQ ID NO: 258 - protein, 127 aa, synthetic construct
  VARIANT 1..3: note = S, GG, AS, ASS or absent
XXXQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSS

SEQ ID NO: 259 - protein, 126 aa, synthetic construct
ASQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSS

SEQ ID NO: 260 - protein, 130 aa, synthetic construct
  VARIANT 1..3: note = S, GG, AS, ASS or absent
XXXQLQLVESGGGLVQAGGSLRLSCAASGRTFSDDTVGWFRQAPGKEREFVAAITWNGDTTYYGDSVKGRFTISRDSAKNTLYLDMNKLEPEDTAVYYCAAAAGQDWSLGYLRESSYAYWGQGTQVTVSS

SEQ ID NO: 261 - protein, 129 aa, synthetic construct
ASQLQLVESGGGLVQAGGSLRLSCAASGRTFSDDTVGWFRQAPGKEREFVAAITWNGDTTYYGDSVKGRFTISRDSAKNTLYLDMNKLEPEDTAVYYCAAAAGQDWSLGYLRESSYAYWGQGTQVTVSS

SEQ ID NO: 262 - protein, 130 aa, synthetic construct
  VARIANT 1..3: note = S, GG, AS, ASS or absent
XXXQLQLVESGGGLVQPGGSLRLSCSASGRTFSDDTVGWFRQAPGKEREFVAAITWNGDTTYYGDSVKGRFTISRDSSKNTLYLQMNSLRAEDTAVYYCAAAAGQDWSLGYLRESSYAYWGQGTLVTVSS

SEQ ID NO: 263 - protein, 129 aa, synthetic construct
ASQLQLVESGGGLVQPGGSLRLSCSASGRTFSDDTVGWFRQAPGKEREFVAAITWNGDTTYYGDSVKGRFTISRDSSKNTLYLQMNSLRAEDTAVYYCAAAAGQDWSLGYLRESSYAYWGQGTLVTVSS

SEQ ID NO: 264 - protein, 130 aa, synthetic construct
  VARIANT 1..3: note = S, GG, AS, ASS or absent
XXXQVQLQESGGGLVQAGDSLRLSCAASGRTFSGYTVGWFRQAPGKEREFVAAITWNGDTTYFGDSVKGRFTISRDSAKNTLYLEMNKLKPEDTAVYYCAAATGQEWSLQYLRESSYAYWGQGTQVTVSS

SEQ ID NO: 265 - protein, 129 aa, synthetic construct
ASQVQLQESGGGLVQAGDSLRLSCAASGRTFSGYTVGWFRQAPGKEREFVAAITWNGDTTYFGDSVKGRFTISRDSAKNTLYLEMNKLKPEDTAVYYCAAATGQEWSLQYLRESSYAYWGQGTQVTVSS

SEQ ID NO: 266 - protein, 127 aa, synthetic construct
EVQLLESGGGLVQPGGSLRLSCAASGRTFSGYTVGWFRQAPGKEREFVAAITWNGDTTYFGDSVKGRFTISRDSSKNTLYLQMNSLRAEDTAVYYCAAATGQEWSLQYLRESSYAYWGQGTLVTVSS

SEQ ID NO: 267 - protein, 128 aa, synthetic construct
  VARIANT 1..3: note = S, GG, AS, ASS or absent
XXXQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSS

SEQ ID NO: 268 - protein, 127 aa, synthetic construct
ASQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSS

SEQ ID NO: 269 - protein, 125 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSS

SEQ ID NO: 270 - protein, 127 aa, synthetic construct
  VARIANT 1..3: note = S, GG, AS, ASS or absent
XXXQVQLVESGGGSVQAGGSLRLSCAASGRTFSSYGMGWFRQAPGKERGFVATINWIPGNTYYADSVKGRFTISGEEDKNTVYLQMDNLKPEDTAVYYCAARRRIDNYYRASEYDYWGQGTQVTVSS

SEQ ID NO: 271 - protein, 126 aa, synthetic construct
ASQVQLVESGGGSVQAGGSLRLSCAASGRTFSSYGMGWFRQAPGKERGFVATINWIPGNTYYADSVKGRFTISGEEDKNTVYLQMDNLKPEDTAVYYCAARRRIDNYYRASEYDYWGQGTQVTVSS

SEQ ID NO: 272 - protein, 127 aa, synthetic construct
  VARIANT 1..3: note = S, GG, AS, ASS or absent
XXXQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSS

SEQ ID NO: 273 - protein, 126 aa, synthetic construct
ASQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSS

SEQ ID NO: 274 - protein, 128 aa, synthetic construct
  VARIANT 1..3: note = S, GG, AS, ASS or absent
XXXQVQLQESGGGSVQAGGSLRLSCAASGRTFSSYGMGWFRQAPGKERGFVATIIGWIPGNTYYADSVKGRFTITGDKDKNTVYLQMDNLKPEDTAVYYCAARRRIDSYYRASEYDYWGQGTQVTVSS

SEQ ID NO: 275 - protein, 127 aa, synthetic construct
ASQVQLQESGGGSVQAGGSLRLSCAASGRTFSSYGMGWFRQAPGKERGFVATIIGWIPGNTYYADSVKGRFTITGDKDKNTVYLQMDNLKPEDTAVYYCAARRRIDSYYRASEYDYWGQGTQVTVSS

SEQ ID NO: 276 - protein, 128 aa, synthetic construct
  VARIANT 1..3: note = S, GG, AS, ASS or absent
XXXQVQLVESGGGVVQPGGSLRLSCAASGRTFSSYGMGWFRQAPGKEREFVATIIGWIPGNTYYADSVKGRFTISGDKSKNTVYLQMNSLRAEDTAVYYCAARRRIDSYYRASEYDYWGQGTLVTVSS

SEQ ID NO: 277 - protein, 127 aa, synthetic construct
ASQVQLVESGGGVVQPGGSLRLSCAASGRTFSSYGMGWFRQAPGKEREFVATIIGWIPGNTYYADSVKGRFTISGDKSKNTVYLQMNSLRAEDTAVYYCAARRRIDSYYRASEYDYWGQGTLVTVSS

SEQ ID NO: 278 - protein, 127 aa, synthetic construct
  VARIANT 1..3: note = S, GG, AS, ASS or absent
XXXQVQLLESGGGLVQAGGSLKLSCTNSGRTFSGYGMGWFRQPPGKEREFVSSINWIYGATYYSDSVKGRFALSRDKAKNTVYLQMDSLKPEDTAVYYCAAKINGESYFNTGSFGSWGQGTQVTVSS

SEQ ID NO: 279 - protein, 126 aa, synthetic construct
ASQVQLLESGGGLVQAGGSLKLSCTNSGRTFSGYGMGWFRQPPGKEREFVSSINWIYGATYYSDSVKGRFALSRDKAKNTVYLQMDSLKPEDTAVYYCAAKINGESYFNTGSFGSWGQGTQVTVSS

SEQ ID NO: 280 - protein, 124 aa, synthetic construct
EVQLLESGGGLVQPGGSLRLSCAASGRTFSGYGMGWFRQAPGKEREFVSSINWIYGATYYSDSVKGRFTLSRDKSKNTVYLQMNSLRAEDTAVYYCAAKINGESYFNTGSFGSWGQGTLVTVSS

SEQ ID NO: 281 - protein, 127 aa, synthetic construct
  VARIANT 1..3: note = S, GG, AS, ASS or absent
XXXQLQLQESGGGLVQAGGSLKLSCTNSGRTFSGYGMGWFRQAPGKEREFVSAINWIYGATYYADSVKGRFAVSGDKAKNTLYLQMNSLKPEDTAVYYCAAKINGGSYFNIGSFGSWGQGTQVTVSS

SEQ ID NO: 282 - protein, 126 aa, synthetic construct
ASQLQLQESGGGLVQAGGSLKLSCTNSGRTFSGYGMGWFRQAPGKEREFVSAINWIYGATYYADSVKGRFAVSGDKAKNTLYLQMNSLKPEDTAVYYCAAKINGGSYFNIGSFGSWGQGTQVTVSS

SEQ ID NO: 283 - protein, 127 aa, synthetic construct
  VARIANT 1..3: note = S, GG, AS, ASS or absent
XXXQLQLVESGGGLVQPGGSLRLSCSASGRTFSGYGMGWFRQAPGKEREFVSAINWIYGATYYADSVKGRFTVSGDKSKNTLYLQMNSLRAEDTAVYYCAAKINGGSYFNIGSFGSWGQGTLVTVSS

SEQ ID NO: 284 - protein, 126 aa, synthetic construct
ASQLQLVESGGGLVQPGGSLRLSCSASGRTFSGYGMGWFRQAPGKEREFVSAINWIYGATYYADSVKGRFTVSGDKSKNTLYLQMNSLRAEDTAVYYCAAKINGGSYFNIGSFGSWGQGTLVTVSS

SEQ ID NO: 285 - protein, 127 aa, synthetic construct
  VARIANT 1..3: note = S, GG, AS, ASS or absent
XXXQVQLVESGGGLVQAGGSLRLSCAASGGTFSYYGMGWFRQAPGKEREFVAAIRWIGGDTYYADSVKGRFTISKDNAKNTLYLQMSSLKPEDTAVYYCAAKTSVTSYLSEKSYNYWGQGTQVTVSS

SEQ ID NO: 286 - protein, 126 aa, synthetic construct
ASQVQLVESGGGLVQAGGSLRLSCAASGGTFSYYGMGWFRQAPGKEREFVAAIRWIGGDTYYADSVKGRFTISKDNAKNTLYLQMSSLKPEDTAVYYCAAKTSVTSYLSEKSYNYWGQGTQVTVSS

SEQ ID NO: 287 - protein, 124 aa, synthetic construct
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSS

SEQ ID NO: 288 - protein, 5 aa, synthetic construct
EGVIG

SEQ ID NO: 289 - protein, 17 aa, synthetic construct
CISARDGSTHYANAVKG

SEQ ID NO: 290 - protein, 15 aa, synthetic construct
SLWGLGCKREEDYDY

SEQ ID NO: 291 - protein, 5 aa, synthetic construct
YPTIG

SEQ ID NO: 292 - protein, 17 aa, synthetic construct
CISASDGSTHYADSVKG

SEQ ID NO: 293 - protein, 15 aa, synthetic construct
SLWGLGCKSEADYDY

SEQ ID NO: 294 - protein, 17 aa, synthetic construct
AISASDASTHYADAVKG

SEQ ID NO: 295 - protein, 15 aa, synthetic construct
SLHGLGAKSEADYDY

SEQ ID NO: 296 - protein, 5 aa, synthetic construct
YSTIG

SEQ ID NO: 297 - protein, 17 aa, synthetic construct
CISASEGSTHYADAVKG

SEQ ID NO: 298 - protein, 15 aa, synthetic construct
SLHGLGCKSEADYDY

SEQ ID NO: 299 - protein, 5 aa, synthetic construct
LDHMG

SEQ ID NO: 300 - protein, 16 aa, synthetic construct
YMFTDGRTQYGEPVKG

SEQ ID NO: 301 - skipped

SEQ ID NO: 302 - protein, 5 aa, synthetic construct
ISTMA

SEQ ID NO: 303 - protein, 17 aa, synthetic construct
RIIWSSGATNYADSVKG

SEQ ID NO: 304 - protein, 12 aa, synthetic construct
AISQTRGTFWTY

SEQ ID NO: 305 - protein, 5 aa, synthetic construct
SYGMS

SEQ ID NO: 306 - protein, 17 aa, synthetic construct
QINTGGGSTSYADSVKG

SEQ ID NO: 307 - protein, 17 aa, synthetic construct
SSRLYSSSWYGSREYDY

SEQ ID NO: 308 - protein, 19 aa, synthetic construct
CISSSGRGVNANYIDSVRG

SEQ ID NO: 309 - protein, 12 aa, synthetic construct
NCWRLTSPAADN

SEQ ID NO: 310 - protein, 5 aa, synthetic construct
YYSIS

SEQ ID NO: 311 - protein, 17 aa, synthetic construct
CISRSGGRINYADSVKG

SEQ ID NO: 312 - protein, 18 aa, synthetic construct
TRLPLSGLCVIGSSDNAL

SEQ ID NO: 313 - protein, 12 aa, synthetic construct
AISQRSGTFWTY

SEQ ID NO: 314 - protein, 5 aa, synthetic construct
GYAVA

SEQ ID NO: 315 - protein, 17 aa, synthetic construct
SISSSGIRTNYKDSVKG

SEQ ID NO: 316 - protein, 12 aa, synthetic construct
KRGPILFGTYTH

SEQ ID NO: 317 - protein, 5 aa, synthetic construct
IFAMG

SEQ ID NO: 318 - protein, 17 aa, synthetic construct
AVTLRSGITSYLDSVKG

SEQ ID NO: 319 - protein, 8 aa, synthetic construct
RDYRSRDY

SEQ ID NO: 320 - protein, 5 aa, synthetic construct
RYAIG

SEQ ID NO: 321 - protein, 17 aa, synthetic construct
CITTSGGGSNYADSVKG

SEQ ID NO: 322 - protein, 18 aa, synthetic construct
TPLNYYCGIDFNPDKYDY

SEQ ID NO: 323 - protein, 5 aa, synthetic construct
FAPMG

SEQ ID NO: 324 - protein, 16 aa, synthetic construct
AINRDFSTYYANSVKG

SEQ ID NO: 325 - protein, 20 aa, synthetic construct
SPGRIWSGTILPPAPDRVVS

SEQ ID NO: 326 - protein, 8 aa, synthetic construct
GISLDEGV

SEQ ID NO: 327 - protein, 8 aa, synthetic construct
ISARDGST

SEQ ID NO: 328 - protein, 17 aa, synthetic construct
AASLWGLGCKREEDYDY

SEQ ID NO: 329 - protein, 8 aa, synthetic construct
GNSLDYPT

SEQ ID NO: 330 - protein, 8 aa, synthetic construct
ISASDGST

SEQ ID NO: 331 - protein, 17 aa, synthetic construct
AASLWGLGCKSEADYDY

SEQ ID NO: 332 - protein, 8 aa, synthetic construct
ISASDAST

SEQ ID NO: 333 - protein, 17 aa, synthetic construct
AASLHGLGAKSEADYDY

SEQ ID NO: 334 - protein, 8 aa, synthetic construct
GNSLDYST

SEQ ID NO: 335 - protein, 8 aa, synthetic construct
ISASEGST

SEQ ID NO: 336 - protein, 17 aa, synthetic construct
AASLHGLGCKSEADYDY

SEQ ID NO: 337 - protein, 8 aa, synthetic construct
GSTSTLDH

SEQ ID NO: 338 - protein, 7 aa, synthetic construct
MFTDGRT

SEQ ID NO: 339 - protein, 5 aa, synthetic construct
GAQGI

SEQ ID NO: 340 - protein, 8 aa, synthetic construct
GSFSSIST

SEQ ID NO: 341 - protein, 8 aa, synthetic construct
IIWSSGAT

SEQ ID NO: 342 - protein, 14 aa, synthetic construct
SGAISQTRGTFWTY

SEQ ID NO: 343 - protein, 8 aa, synthetic construct
GFAFSSYG

SEQ ID NO: 344 - protein, 8 aa, synthetic construct
INTGGGST

SEQ ID NO: 345 - protein, 19 aa, synthetic construct
AASSRLYSSSWYGSREYDY

SEQ ID NO: 346 - protein, 8 aa, synthetic construct
GFTSPYST

SEQ ID NO: 347 - protein, 10 aa, synthetic construct
ISSSGRGVNA

SEQ ID NO: 348 - protein, 14 aa, synthetic construct
ATNCWRLTSPAADN

SEQ ID NO: 349 - protein, 8 aa, synthetic construct
GLTLDYYS

SEQ ID NO: 350 - protein, 8 aa, synthetic construct
ISRSGGRI

SEQ ID NO: 351 - protein, 20 aa, synthetic construct
AATRLPLSGLCVIGSSDNAL

SEQ ID NO: 352 - protein, 8 aa, synthetic construct
GNFSSIST

SEQ ID NO: 353 - protein, 14 aa, synthetic construct
AGAISQRSGTFWTY

SEQ ID NO: 354 - protein, 8 aa, synthetic construct
ISTLNGYA

SEQ ID NO: 355 - protein, 8 aa, synthetic construct
ISSSGIRT

SEQ ID NO: 356 - protein, 14 aa, synthetic construct
AAKRGPILFGTYTH

SEQ ID NO: 357 - protein, 8 aa, synthetic construct
GSALSIFA

SEQ ID NO: 358 - protein, 8 aa, synthetic construct
VTLRSGIT

SEQ ID NO: 359 - protein, 10 aa, synthetic construct
NVRDYRSRDY

SEQ ID NO: 360 - protein, 8 aa, synthetic construct
GFTVDRYA

SEQ ID NO: 361 - protein, 8 aa, synthetic construct
ITTSGGGS

SEQ ID NO: 362 - protein, 20 aa, synthetic construct
AATPLNYYCGIDFNPDKYDY

SEQ ID NO: 363 - protein, 8 aa, synthetic construct
ETIINFAP

SEQ ID NO: 364 - protein, 7 aa, synthetic construct
INRDFST

SEQ ID NO: 365 - protein, 22 aa, synthetic construct
AASPGRIWSGTILPPAPDRVVS

SEQ ID NO: 366 - protein, 7 aa, synthetic construct
GISLDEG

SEQ ID NO: 367 - protein, 4 aa, synthetic construct
ARDG

SEQ ID NO: 368 - protein, 13 aa, synthetic construct
LWGLGCKREEDYD

SEQ ID NO: 369 - protein, 7 aa, synthetic construct
GNSLDYP

SEQ ID NO: 370 - protein, 4 aa, synthetic construct
ASDG

SEQ ID NO: 371 - protein, 13 aa, synthetic construct
LWGLGCKSEADYD

SEQ ID NO: 372 - protein, 4 aa, synthetic construct
ASDA

SEQ ID NO: 373 - protein, 13 aa, synthetic construct
LHGLGAKSEADYD

SEQ ID NO: 374 - protein, 7 aa, synthetic construct
GNSLDYS

SEQ ID NO: 375 - protein, 4 aa, synthetic construct
ASEG

SEQ ID NO: 376 - protein, 13 aa, synthetic construct
LHGLGCKSEADYD

SEQ ID NO: 377 - protein, 7 aa, synthetic construct
GSTSTLD

SEQ ID NO: 378-379 - skipped (2 entries)

SEQ ID NO: 380 - protein, 7 aa, synthetic construct
GSFSSIS

SEQ ID NO: 381 - protein, 4 aa, synthetic construct
WSSG

SEQ ID NO: 382 - protein, 10 aa, synthetic construct
ISQTRGTFWT

SEQ ID NO: 383 - protein, 7 aa, synthetic construct
GFAFSSY

SEQ ID NO: 384 - protein, 4 aa, synthetic construct
TGGG

SEQ ID NO: 385 - protein, 15 aa, synthetic construct
SRLYSSSWYGSREYD

SEQ ID NO: 386 - protein, 7 aa, synthetic construct
GFTSPYS

SEQ ID NO: 387 - protein, 6 aa, synthetic construct
SSGRGV

SEQ ID NO: 388 - protein, 10 aa, synthetic construct
CWRLTSPAAD

SEQ ID NO: 389 - protein, 7 aa, synthetic construct
GLTLDYY

SEQ ID NO: 390 - protein, 4 aa, synthetic construct
RSGG

SEQ ID NO: 391 - protein, 16 aa, synthetic construct
RLPLSGLCVIGSSDNA

SEQ ID NO: 392 - protein, 7 aa, synthetic construct
GNFSSIS

SEQ ID NO: 393 - protein, 10 aa, synthetic construct
ISQRSGTFWT

SEQ ID NO: 394 - protein, 7 aa, synthetic construct
ISTLNGY

SEQ ID NO: 395 - protein, 4 aa, synthetic construct
SSGI

SEQ ID NO: 396 - protein, 10 aa, synthetic construct
RGPILFGTYT

SEQ ID NO: 397 - protein, 7 aa, synthetic construct
GSALSIF

SEQ ID NO: 398 - protein, 4 aa, synthetic construct
LRSG

SEQ ID NO: 399 - protein, 6 aa, synthetic construct
DYRSRD

SEQ ID NO: 400 - protein, 7 aa, synthetic construct
GFTVDRY

SEQ ID NO: 401 - protein, 4 aa, synthetic construct
TSGG

SEQ ID NO: 402 - protein, 16 aa, synthetic construct
PLNYYCGIDFNPDKYD

SEQ ID NO: 403 - protein, 7 aa, synthetic construct
ETIINFA

SEQ ID NO: 404 - skipped

SEQ ID NO: 405 - protein, 18 aa, synthetic construct
PGRIWSGTILPPAPDRVV

SEQ ID NO: 406 - protein, 10 aa, synthetic construct
ASGISLDEGV

SEQ ID NO: 407 - protein, 17 aa, synthetic construct
ISARDGSTHYANAVKGR

SEQ ID NO: 408 - protein, 14 aa, synthetic construct
SLWGLGCKREEDYD

SEQ ID NO: 409 - protein, 10 aa, synthetic construct
ASGNSLDYPT

SEQ ID NO: 410 - protein, 17 aa, synthetic construct
ISASDGSTHYADSVKGR

SEQ ID NO: 411 - protein, 14 aa, synthetic construct
SLWGLGCKSEADYD

SEQ ID NO: 412 - protein, 17 aa, synthetic construct
ISASDASTHYADAVKGR

SEQ ID NO: 413 - protein, 14 aa, synthetic construct
SLHGLGAKSEADYD

SEQ ID NO: 414 - protein, 10 aa, synthetic construct
ASGNSLDYST

SEQ ID NO: 415 - protein, 17 aa, synthetic construct
ISASEGSTHYADAVKGR

SEQ ID NO: 416 - protein, 14 aa, synthetic construct
SLHGLGCKSEADYD

SEQ ID NO: 417 - protein, 10 aa, synthetic construct
ASGSTSTLDH

SEQ ID NO: 418 - protein, 16 aa, synthetic construct
MFTDGRTQYGEPVKGR

SEQ ID NO: 419 - skipped

SEQ ID NO: 420 - protein, 10 aa, synthetic construct
ASGSFSSIST

SEQ ID NO: 421 - protein, 17 aa, synthetic construct
IIWSSGATNYADSVKGR

SEQ ID NO: 422 - protein, 11 aa, synthetic construct
AISQTRGTFWT

SEQ ID NO: 423 - protein, 10 aa, synthetic construct
ASGFAFSSYG

SEQ ID NO: 424 - protein, 17 aa, synthetic construct
INTGGGSTSYADSVKGR

SEQ ID NO: 425 - protein, 16 aa, synthetic construct
SSRLYSSSWYGSREYD

SEQ ID NO: 426 - protein, 10 aa, synthetic construct
ASGFTSPYST

SEQ ID NO: 427 - protein, 19 aa, synthetic construct
ISSSGRGVNANYIDSVRGR

SEQ ID NO: 428 - protein, 11 aa, synthetic construct
NCWRLTSPAAD

SEQ ID NO: 429 - protein, 10 aa, synthetic construct
ASGLTLDYYS

SEQ ID NO: 430 - protein, 17 aa, synthetic construct
ISRSGGRINYADSVKGR

SEQ ID NO: 431 - protein, 17 aa, synthetic construct
TRLPLSGLCVIGSSDNA

SEQ ID NO: 432 - protein, 10 aa, synthetic construct
ASGNFSSIST

SEQ ID NO: 433 - protein, 11 aa, synthetic construct
AISQRSGTFWT

SEQ ID NO: 434 - protein, 10 aa, synthetic construct
SSISTLNGYA

SEQ ID NO: 435 - protein, 17 aa, synthetic construct
ISSSGIRTNYKDSVKGR

SEQ ID NO: 436 - protein, 11 aa, synthetic construct
KRGPILFGTYT

SEQ ID NO: 437 - protein, 10 aa, synthetic construct
ASGSALSIFA

SEQ ID NO: 438 - protein, 17 aa, synthetic construct
VTLRSGITSYLDSVKGR

SEQ ID NO: 439 - protein, 7 aa, synthetic construct
RDYRSRD

SEQ ID NO: 440 - protein, 10 aa, synthetic construct
ASGFTVDRYA

SEQ ID NO: 441 - protein, 17 aa, synthetic construct
ITTSGGGSNYADSVKGR

SEQ ID NO: 442 - protein, 17 aa, synthetic construct
TPLNYYCGIDFNPDKYD

SEQ ID NO: 443 - protein, 10 aa, synthetic construct
ASETIINFAP

SEQ ID NO: 444 - protein, 16 aa, synthetic construct
INRDFSTYYANSVKGR

SEQ ID NO: 445 - protein, 19 aa, synthetic construct
SPGRIWSGTILPPAPDRVV

SEQ ID NO: 446 - protein, 124 aa, synthetic construct
QVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 447 - protein, 124 aa, synthetic construct
EVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 448 - protein, 124 aa, synthetic construct
  VARIANT 1: note = E or Q
  VARIANT 5: note = L or Q
  VARIANT 19: note = K or R
  VARIANT 27: note = I or N
  VARIANT 31: note = E or Y
  VARIANT 32: note = G, P or S
  VARIANT 33: note = V or T
  VARIANT 39: note = Q or R
  VARIANT 44: note = E or G
  VARIANT 45: note = L or R
  VARIANT 50: note = A or C
  VARIANT 54: note = R or S
  VARIANT 56: note = A or G
  VARIANT 62: note = D or N
  VARIANT 63: note = A or S
  VARIANT 75: note = S or T
  VARIANT 80: note = D or Y
  VARIANT 85: note = N or S
  VARIANT 87: note = K or R
  VARIANT 88: note = A or P
  VARIANT 91: note = A or T
  VARIANT 92: note = A or G
  VARIANT 93: note = H or V
  VARIANT 101: note = H or W
  VARIANT 105: note = A or C
  VARIANT 107: note = R or S
  VARIANT 119: note = L, M or Q
  VARIANT 109: note = A or E
XVQLXESGGGLVQPGGSLXLSCAASGXSLDXXXIGWFRXAPGKXXEGVSXISAXDXSTHYAXXVKGRFTISRDNXKNTVXLQMNXLXXEDXXXYYCAASLXGLGXKXEXDYDYWGQGTXVTVSS

SEQ ID NO: 449 - protein, 124 aa, synthetic construct
  VARIANT 1: note = E or Q
  VARIANT 44: note = E or G
  VARIANT 45: note = L or R
  VARIANT 50: note = A or C
  VARIANT 56: note = A or G
  VARIANT 63: note = A or S
  VARIANT 75: note = S or T
  VARIANT 80: note = D or Y
  VARIANT 85: note = N or S
  VARIANT 87: note = K or R
  VARIANT 88: note = A or P
  VARIANT 92: note = A or G
  VARIANT 93: note = H or V
  VARIANT 101: note = H or W
  VARIANT 105: note = A or C
  VARIANT 119: note = M or Q
XVQLVESGGGLVQPGGSLRLSCAASGNSLDYPTIGWFRQAPGKXXEGVSXISASDXSTHYADXVKGRFTISRDNXKNTVXLQMNXLXXEDTXXYYCAASLXGLGXKSEADYDYWGQGTXVTVSS

SEQ ID NO: 450 - protein, 124 aa, synthetic construct
QVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 451 - protein, 124 aa, synthetic construct
EVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 452 - protein, 124 aa, synthetic construct
EVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 453 - protein, 111 aa, synthetic construct
QVQLVESGGGVVQAGGSLRLSCAASGSTSTLDHMGWYRQAPGKSRELVGYMFTDGRTQYGEPVKGRFTVSRDNGRNTIFLQMNNLKLEDTAVYYCGAQGIWGQGTQVTVSS

SEQ ID NO: 454 - protein, 111 aa, synthetic construct
QVQLVESGGGVVQPGGSLRLSCAASGSTSTLDHMGWYRQAPGKSRELVGYMFTDGRTQYGEPVKGRFTVSRDNSKNTIYLQMNSLRAEDTAVYYCGAQGIWGQGTMVTVSS

SEQ ID NO: 455 - protein, 121 aa, synthetic construct
  VARIANT 1: note = E or Q
  VARIANT 5: note = L, Q or V
  VARIANT 13: note = K or Q
  VARIANT 14: note = A or P
  VARIANT 27: note = N or S
  VARIANT 48: note = L or V
  VARIANT 40: note = A or V
  VARIANT 70: note = A or I
  VARIANT 75: note = A, S or T
  VARIANT 84: note = D or N
  VARIANT 85: note = R or S
  VARIANT 87: note = R or T
  VARIANT 88: note = A or P
  VARIANT 95: note = L or Y
  VARIANT 103: note = R or T
  VARIANT 111: note = K or W
  VARIANT 116: note = L or Q
XVQLXESGGGLVXXGGSLRLSCAASGXFSSISTMAWYRQXPGKQREWXARIIWSSGATNYADSVKGRFTXSRDNXKNTMYLQMXXLXXEDTAAYXCSGAISQXRGTFWTYXSQGTXVTVSS

SEQ ID NO: 456 - protein, 121 aa, synthetic construct
EVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSS

SEQ ID NO: 457 - protein, 121 aa, synthetic construct
EVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSS

SEQ ID NO: 458 - protein, 126 aa, synthetic construct
QVQLQESGGGLVQPGGSLRLSCAASGFAFSSYGMSWVRQAPGKGLEWVSQINTGGGSTSYADSVKGRFTISRDNAKNTLYLQMNSLKPEDTAVYYCAASSRLYSSSWYGSREYDYWGQGTQVTVSS

SEQ ID NO: 459 - protein, 126 aa, synthetic construct
EVQLLESGGGLVQPGGSLRLSCAASGFAFSSYGMSWVRQAPGKGLEWVSQINTGGGSTSYADSVKGRFTISRDNSKNTLYLQMNSLRAEDTAVYYCAASSRLYSSSWYGSREYDYWGQGTLVTVSS

SEQ ID NO: 460 - protein, 123 aa, synthetic construct
  VARIANT 1: note = E or Q
  VARIANT 14: note = P or S
  VARIANT 23: note = S or V
  VARIANT 44: note = E or G
  VARIANT 77: note = A or S
  VARIANT 87: note = K or S
  VARIANT 90: note = A or P
  VARIANT 91: note = D or E
  VARIANT 95: note = I or V
  VARIANT 118: note = M or Q
XVQLVESGGGLVQXGGSLRLSCXASGFTSPYSTIGWFRQAPGKXREGVACISSSGRGVNANYIDSVRGRFTISRDNXKNTVYLQMNXLKXXDTAXYYCATNCWRLTSPAADNWGQGTXVTVSS

SEQ ID NO: 461 - protein, 123 aa, synthetic construct
QVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 462 - protein, 123 aa, synthetic construct
EVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 463 - protein, 127 aa, synthetic construct
DLQLVESGGGLVQPGGSLRLSCAASGLTLDYYSISWFRQGPGNEREAVSCISRSGGRINYADSVKGRFTISRDSAKNTVYLQMNSLKPEDTAVYYCAATRLPLSGLCVIGSSDNALWGQGTQVTVSS

SEQ ID NO: 464 - protein, 127 aa, synthetic construct
ELQLVESGGGLVQPGGSLRLSCAASGLTLDYYSISWFRQAPGKEREAVSCISRSGGRINYADSVKGRFTISRDSSKNTVYLQMNSLRAEDTAVYYCAATRLPLSGLCVIGSSDNALWGQGTLVTVSS

SEQ ID NO: 465 - protein, 121 aa, synthetic construct
QVQLQESGGGLVQAGGSLRLSCAASGNFSSISTMAWYRQVPGKQREWVARIIWSSGATNYADSVKGRFTASRDNTKNTMYLQMDRLTPEDTAVYLCAGAISQRSGTFWTYWGQGTQVTVSS

SEQ ID NO: 466 - protein, 121 aa, synthetic construct
QVQLLESGGGLVKPGGSLRLSCAASGNFSSISTMAWYRQAPGKQREWVARIIWSSGATNYADSVKGRFTASRDNAKNSMYLQMNSLRAEDTAVYYCAGAISQRSGTFWTYWGQGTLVTVSS

SEQ ID NO: 467 - protein, 121 aa, synthetic construct
QLQLVESGGGLVQPGGSLTLSCASSISTLNGYAVAWFRQAPGKERSAVSSISSSGIRTNYKDSVKGRFTIWRDNAKAAVYLQLNNLEPEDTAMYYCAAKRGPILFGTYTHWGQGTQVTVSS

SEQ ID NO: 468 - protein, 121 aa, synthetic construct
QLQLVESGGGLVKPGGSLRLSCAASGSTLNGYAVAWFRQAPGKEREAVSSISSSGIRTNYKDSVKGRFTISRDNAKNSVYLQMNSLRAEDTAVYYCAAKRGPILFGTYTHWGQGTLVTVSS

SEQ ID NO: 469 - protein, 117 aa, synthetic construct
EVQLVESGGGLVQAGGSLRLSCAASGSALSIFAMGWYRQTPGKQSVSVAAVTLRSGITSYLDSVKGRFIISIDRAKNTMYLEMNDLKPEDTAIYYCNVRDYRSRDYWGQGTQVTVSS

SEQ ID NO: 470 - protein, 117 aa, synthetic construct
EVQLVESGGGLVQPGGSLRLSCAASGSALSIFAMGWYRQAPGKQSESVAAVTLRSGITSYLDSVKGRFTISIDRSKNTMYLQMNSLRAEDTAVYYCNVRDYRSRDYWGQGTLVTVSS

SEQ ID NO: 471 - protein, 127 aa, synthetic construct
QVQLQESGGGLVQPGGSLRLSCAASGFTVDRYAIGWFRQAPGKEREGVQCITTSGGGSNYADSVKGRFTIARDNAKNTVYLQMDSLKPEDTAIYYCAATPLNYYCGIDFNPDKYDYWGQGTQVTVSS

SEQ ID NO: 472 - protein, 127 aa, synthetic construct
EVQLLESGGGLVQPGGSLRLSCAASGFTVDRYAIGWFRQAPGKEREGVQCITTSGGGSNYADSVKGRFTISRDNSKNTVYLQMNSLRAEDTAVYYCAATPLNYYCGIDFNPDKYDYWGQGTMVTVSS

SEQ ID NO: 473 - protein, 128 aa, synthetic construct
QVQLVESGGGLVQPGGSLRLSCVASETIINFAPMGWYRQAPGTERELVAAINRDFSTYYANSVKGRFTISRDNAKKMLSLQMNSLNPEDTAMYYCAASPGRIWSGTILPPAPDRVVSWGQGTQVTVSS

SEQ ID NO: 474 - protein, 128 aa, synthetic construct
QVQLVESGGGLVQPGGSLRLSCSASGTIINFAPMGWYRQAPGKERELVAAINRDFSTYYANSVKGRFTISRDNSKNTLYLQMNSLRAEDTAVYYCAASPGRIWSGTILPPAPDRVVSWGQGTMVTVSS

SEQ ID NO: 475 - protein, 259 aa, synthetic construct
  VARIANT 1..3: note = S, GG, AS, ASS or absent
  VARIANT 4: note = E or Q
  VARIANT 16: note = Q or R
  VARIANT 17: note = A or P
  VARIANT 19: note = D or G
  VARIANT 47: note = E or G
  VARIANT 52: note = A or S
  VARIANT 57: note = I or Y
  VARIANT 59: note = D or E
  VARIANT 60: note = A, G or S
  VARIANT 65: note = D or E
  VARIANT 77: note = N or S
  VARIANT 78: note = A or S
  VARIANT 87: note = N or S
  VARIANT 90: note = K or R
  VARIANT 91: note = A or P
  VARIANT 104: note = R or S
  VARIANT 105: note = P or V
  VARIANT 106: note = D or T
  VARIANT 115: note = G or N
  VARIANT 122: note = M or Q
  VARIANT 128..135: note = GGGS, GGGGS, or GGGSGGGS
  VARIANT 136: note = E or Q
  VARIANT 140: note = L or Q
  VARIANT 154: note = K or R
  VARIANT 162: note = I or N
  VARIANT 166: note = E or Y
  VARIANT 167: note = G, P or S
  VARIANT 168: note = V or T
  VARIANT 174: note = Q or R
  VARIANT 179: note = E or G
  VARIANT 180: note = L or R
  VARIANT 185: note = A or C
  VARIANT 189: note = R or S
  VARIANT 191: note = A or G
  VARIANT 197: note = D or N
  VARIANT 198: note = A or S
  VARIANT 210: note = S or T
  VARIANT 215: note = D or Y
  VARIANT 220: note = N or S
  VARIANT 222: note = K or R
  VARIANT 223: note = A or P
  VARIANT 226: note = A or T
  VARIANT 227: note = A or G
  VARIANT 228: note = H or V
  VARIANT 236: note = H or W
  VARIANT 240: note = A or C
  VARIANT 242: note = R or S
  VARIANT 244: note = A or E
  VARIANT 254: note = L, M or Q
XXXXLQLVESGGGLVXXGXSLRLSCAASGRTFSAYGMGWFRQAPGKXREFVXAIRWXGXXTYYAXSVKGRFTISKDXXKNTLYLQMXSLXXEDTAVYYCAAKTXXXSYLSEKSYXYWGQGTXVTVSSXXXXXXXXXVQLXESGGGLVQPGGSLXLSCAASGXSLDXXXIGWFRXAPGKXXEGVSXISAXDXSTHYAXXVKGRFTISRDNXKNTVXLQMNXLXXEDXXXYYCAASLXGLGXKXEXDYDYWGQGTXVTVSS

SEQ ID NO: 476 - protein, 259 aa, synthetic construct
  VARIANT 1..3: note = S, GG, AS, ASS or absent
  VARIANT 16: note = Q or R
  VARIANT 17: note = A or P
  VARIANT 47: note = E or G
  VARIANT 52: note = A or S
  VARIANT 57: note = I or Y
  VARIANT 59: note = D or E
  VARIANT 60: note = A, G or S
  VARIANT 65: note = D or E
  VARIANT 78: note = A or S
  VARIANT 87: note = N or S
  VARIANT 90: note = K or R
  VARIANT 91: note = A or P
  VARIANT 104: note = R or S
  VARIANT 105: note = P or V
  VARIANT 106: note = D or T
  VARIANT 115: note = G or N
  VARIANT 122: note = M or Q
  VARIANT 128..135: note = GGGS, GGGGS, or GGGSGGGS
  VARIANT 136: note = E or Q
  VARIANT 179: note = E or G
  VARIANT 180: note = L or R
  VARIANT 185: note = A or C
  VARIANT 191: note = A or G
  VARIANT 198: note = A or S
  VARIANT 210: note = S or T
  VARIANT 215: note = D or Y
  VARIANT 220: note = N or S
  VARIANT 222: note = K or R
  VARIANT 223: note = A or P
  VARIANT 227: note = A or G
  VARIANT 228: note = H or V
  VARIANT 236: note = H or W
  VARIANT 240: note = A or C
  VARIANT 254: note = M or Q
XXXQLQLVESGGGLVXXGGSLRLSCAASGRTFSAYGMGWFRQAPGKXREFVXAIRWXGXXTYYAXSVKGRFTISKDNXKNTLYLQMXSLXXEDTAVYYCAAKTXXXSYLSEKSYXYWGQGTXVTVSSXXXXXXXXXVQLVESGGGLVQPGGSLRLSCAASGNSLDYPTIGWFRQAPGKXXEGVSXISASDXSTHYADXVKGRFTISRDNXKNTVXLQMNXLXXEDTXXYYCAASLXGLGXKSEADYDYWGQGTXVTVSS

SEQ ID NO: 477 - protein, 259 aa, synthetic construct
  VARIANT 1..3: note = S, GG, AS, ASS or absent
  VARIANT 128..135: note = GGGS, GGGGS, or GGGSGGGS
XXXQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 478 - protein, 254 aa, synthetic construct
ASQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 479 - protein, 259 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 480 - protein, 254 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 481 - protein, 259 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 482 - protein, 254 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 483 - protein, 259 aa, synthetic construct
  VARIANT 1..3: note = S, GG, AS, ASS or absent
  VARIANT 128..135: note = GGGS, GGGGS, or GGGSGGGS
XXXQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 484 - protein, 254 aa, synthetic construct
ASQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 485 - protein, 259 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 486 - protein, 254 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 487 - protein, 259 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 488 - protein, 254 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 489 - protein, 258 aa, synthetic construct
  VARIANT 1..3: note = S, GG, AS, ASS or absent
  VARIANT 128..135: note = GGGS, GGGGS, or GGGSGGGS
XXXQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 490 - protein, 253 aa, synthetic construct
ASQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 491 - protein, 258 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 492 - protein, 253 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 493 - protein, 258 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 494 - protein, 253 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 495 - protein, 259 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 496 - protein, 254 aa, synthetic construct
ASQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 497 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 498 - protein, 252 aa, synthetic construct
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 499 - protein, 259 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 500 - protein, 254 aa, synthetic construct
ASQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 501 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 502 - protein, 252 aa, synthetic construct
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 503 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 504 - protein, 252 aa, synthetic construct
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 505 - protein, 259 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 506 - protein, 254 aa, synthetic construct
ASQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 507 - protein, 256 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 508 - protein, 252 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 509 - protein, 259 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 510 - protein, 254 aa, synthetic construct
ASQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 511 - protein, 256 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 512 - protein, 252 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 513 - protein, 256 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 514 - protein, 252 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 515 - protein, 258 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 516 - protein, 253 aa, synthetic construct
ASQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 517 - protein, 255 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 518 - protein, 251 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 519 - protein, 259 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 520 - protein, 254 aa, synthetic construct
ASQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 521 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 522 - protein, 252 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 523 - protein, 258 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 524 - protein, 253 aa, synthetic construct
ASQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 525 - protein, 255 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 526 - protein, 251 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 527 - protein, 259 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 528 - protein, 254 aa, synthetic construct
ASQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 529 - protein, 256 aa, synthetic construct; identical to SEQ ID NO: 521
  VARIANT 125..132: note = GGGS, GGGGS, or GGGSGGGS

SEQ ID NO: 530 - protein, 252 aa, synthetic construct; identical to SEQ ID NO: 522

SEQ ID NO: 531 - protein, 259 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 532 - protein, 254 aa, synthetic construct
ASQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 533 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 534 - protein, 252 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 535 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 536 - protein, 252 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 537 - protein, 260 aa, synthetic construct
  VARIANT 1..3: note = S, GG, AS, ASS or absent
  VARIANT 129..136: note = GGGS, GGGGS, or GGGSGGGS
XXXQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 538 - protein, 255 aa, synthetic construct
ASQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 539 - protein, 257 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 540 - protein, 253 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 541 - protein, 260 aa, synthetic construct
  VARIANT 1..3: note = S, GG, AS, ASS or absent
  VARIANT 129..136: note = GGGS, GGGGS, or GGGSGGGS
XXXQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 542 - protein, 255 aa, synthetic construct
ASQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 543 - protein, 257 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 544 - protein, 253 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 545 - protein, 257 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 546 - protein, 253 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 547 - protein, 259 aa, synthetic construct
  VARIANT 1..3: note = S, GG, AS, ASS or absent
  VARIANT 129..136: note = GGGS, GGGGS, or GGGSGGGS
XXXQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 548 - protein, 254 aa, synthetic construct
ASQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 549 - protein, 256 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 550 - protein, 252 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 551 - protein, 259 aa, synthetic construct
  VARIANT 1..3: note = S, GG, AS, ASS or absent
  VARIANT 128..135: note = GGGS, GGGGS, or GGGSGGGS
XXXQVQLVESGGGSVQAGGSLRLSCAASGRTFSSYGMGWFRQAPGKERGFVATINWIPGNTYYADSVKGRFTISGEEDKNTVYLQMDNLKPEDTAVYYCAARRRIDNYYRASEYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 552 - protein, 254 aa, synthetic construct
ASQVQLVESGGGSVQAGGSLRLSCAASGRTFSSYGMGWFRQAPGKERGFVATINWIPGNTYYADSVKGRFTISGEEDKNTVYLQMDNLKPEDTAVYYCAARRRIDNYYRASEYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 553 - protein, 259 aa, synthetic construct
  VARIANT 1..3: note = S, GG, AS, ASS or absent
  VARIANT 128..135: note = GGGS, GGGGS, or GGGSGGGS
XXXQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 554 - protein, 254 aa, synthetic construct
ASQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 555 - protein, 259 aa, synthetic construct
  VARIANT 1..3: note = S, GG, AS, ASS or absent
  VARIANT 128..135: note = GGGS, GGGGS, or GGGSGGGS
XXXQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 556 - protein, 254 aa, synthetic construct
ASQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 557 - protein, 259 aa, synthetic construct
  VARIANT 1..3: note = S, GG, AS, ASS or absent
  VARIANT 128..135: note = GGGS, GGGGS, or GGGSGGGS
XXXQVQLVESGGGLVQAGGSLRLSCAASGGTFSYYGMGWFRQAPGKEREFVAAIRWIGGDTYYADSVKGRFTISKDNAKNTLYLQMSSLKPEDTAVYYCAAKTSVTSYLSEKSYNYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 558 - protein, 254 aa, synthetic construct
ASQVQLVESGGGLVQAGGSLRLSCAASGGTFSYYGMGWFRQAPGKEREFVAAIRWIGGDTYYADSVKGRFTISKDNAKNTLYLQMSSLKPEDTAVYYCAAKTSVTSYLSEKSYNYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 559 - protein, 256 aa, synthetic construct
  VARIANT 125..132: note = GGGS, GGGGS, or GGGSGGGS
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 560 - protein, 252 aa, synthetic construct
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 561 - protein, 256 aa, synthetic construct
  VARIANT 125..132: note = GGGS, GGGGS, or GGGSGGGS
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 562 - protein, 252 aa, synthetic construct
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 563 - protein, 259 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLSLSCAASGRTFSSYGMGWFRQAPGKEREFVAAISWIGSATDYADSVRGRFTISGDNAKNTVYLLMNNLKPEDTAVYYCAARRSPMSRTSVLQYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 564 - protein, 254 aa, synthetic construct
ASQVQLQESGGGLVQAGGSLSLSCAASGRTFSSYGMGWFRQAPGKEREFVAAISWIGSATDYADSVRGRFTISGDNAKNTVYLLMNNLKPEDTAVYYCAARRSPMSRTSVLQYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 565 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 566 - protein, 252 aa, synthetic construct
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 567 - protein, 256 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 568 - protein, 252 aa, synthetic construct
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 569 - protein, 259 aa, synthetic construct
  VARIANT 1..3: note = S, GG, AS, ASS or absent
  VARIANT 128..135: note = GGGS, GGGGS, or GGGSGGGS
XXXQVQLVESGGGLVQAGGSLRLSCAASGRTFSSYGMGWFRQAPGKEREFVAAISWIGNSTYYADSVKGRFTLSGDNAKNTLYLQMNSLKPEDTAVYYCAAKYYTDSYYAAASYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 570 - protein, 254 aa, synthetic construct
ASQVQLVESGGGLVQAGGSLRLSCAASGRTFSSYGMGWFRQAPGKEREFVAAISWIGNSTYYADSVKGRFTLSGDNAKNTLYLQMNSLKPEDTAVYYCAAKYYTDSYYAAASYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSS

SEQ ID NO: 571 - protein, 256 aa, synthetic construct
  VARIANT 125..132: note = GGGS, GGGGS, or GGGSGGGS
EVQLVESGGGLVQPGGSLRLSCAASGRTFSSYGMGWFRQAPGKEREFVAAISWIGNSTYYADSVKGRFTLSGDNSKNTLYLQMNSLRAEDTAVYYCAAKYYTDSYYAAASYAYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 572 - protein, 252 aa, synthetic construct
EVQLVESGGGLVQPGGSLRLSCAASGRTFSSYGMGWFRQAPGKEREFVAAISWIGNSTYYADSVKGRFTLSGDNSKNTLYLQMNSLRAEDTAVYYCAAKYYTDSYYAAASYAYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSS

SEQ ID NO: 573 - protein, 258 aa, synthetic construct
  VARIANT 2: note = L or V
  VARIANT 5: note = L or V
  VARIANT 14: note = A or P
  VARIANT 24: note = A or N
  VARIANT 44: note = E or G
  VARIANT 69: note = S or T
  VARIANT 49: note = A or E
  VARIANT 74: note = N or S
  VARIANT 75: note = A or S
  VARIANT 82: note = E or Q
  VARIANT 87: note = K or R
  VARIANT 88: note = A or P
  VARIANT 122: note = L or Q
  VARIANT 128..135: note = GGGS, GGGGS, or GGGSGGGS
  VARIANT 136: note = E or Q
  VARIANT 149: note = P or S
  VARIANT 158: note = S or V
  VARIANT 179: note = E or G
  VARIANT 212: note = A or S
  VARIANT 222: note = K or S
  VARIANT 225: note = A or P
  VARIANT 226: note = D or E
  VARIANT 230: note = I or V
  VARIANT 253: note = M or Q
  VARIANT 85: note = K or S
EXQLXESGGGLVQXGGSLRLSCAXSGRTFSSHTVGWFRQAPGKXREFVXAITWNGDTTYFGDFVEGRFXISRDXXKNTLYLXMNXLXXEDTAVYYCAAATGQVWSLQYLRESSYAYWGQGTXVTVSSXXXXXXXXXVQLVESGGGLVQXGGSLRLSCXASGFTSPYSTIGWFRQAPGKXREGVACISSSGRGVNANYIDSVRGRFTISRDNXKNTVYLQMNXLKXXDTAXYYCATNCWRLTSPAADNWGQGTXVTVSS

SEQ ID NO: 574 - protein, 258 aa, synthetic construct
  VARIANT 128..135: note = GGGS, GGGGS, or GGGSGGGS
ELQLVESGGGLVQAGGSLRLSCANSGRTFSSHTVGWFRQAPGKEREFVAAITWNGDTTYFGDFVEGRFSISRDSAKNTLYLEMNKLKPEDTAVYYCAAATGQVWSLQYLRESSYAYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 575 - protein, 254 aa, synthetic construct
ELQLVESGGGLVQAGGSLRLSCANSGRTFSSHTVGWFRQAPGKEREFVAAITWNGDTTYFGDFVEGRFSISRDSAKNTLYLEMNKLKPEDTAVYYCAAATGQVWSLQYLRESSYAYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 576 - protein, 258 aa, synthetic construct
  VARIANT 128..135: note = GGGS, GGGGS, or GGGSGGGS
EVQLLESGGGLVQPGGSLRLSCAASGRTFSSHTVGWFRQAPGKGREFVAAITWNGDTTYFGDFVEGRFTISRDNSKNTLYLQMNSLRAEDTAVYYCAAATGQVWSLQYLRESSYAYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 577 - protein, 254 aa, synthetic construct
EVQLLESGGGLVQPGGSLRLSCAASGRTFSSHTVGWFRQAPGKGREFVAAITWNGDTTYFGDFVEGRFTISRDNSKNTLYLQMNSLRAEDTAVYYCAAATGQVWSLQYLRESSYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 578 - protein, 258 aa, synthetic construct
  VARIANT 128..135: note = GGGS, GGGGS, or GGGSGGGS
ELQLLESGGGLVQPGGSLRLSCAASGRTFSSHTVGWFRQAPGKGREFVSAITWNGDTTYFGDFVEGRFTISRDNSKNTLYLQMNSLRAEDTAVYYCAAATGQVWSLQYLRESSYAYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 579 - protein, 254 aa, synthetic construct
ELQLLESGGGLVQPGGSLRLSCAASGRTFSSHTVGWFRQAPGKGREFVSAITWNGDTTYFGDFVEGRFTISRDNSKNTLYLQMNSLRAEDTAVYYCAAATGQVWSLQYLRESSYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 580 - protein, 256 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSXXXXXXXXEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSS

SEQ ID NO: 581 - protein, 251 aa, synthetic construct
ASQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSGGGSEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSS

SEQ ID NO: 582 - protein, 256 aa, synthetic construct
  VARIANT 1..3: note = S, GG, AS, ASS or absent
  VARIANT 128..135: note = GGGS, GGGGS, or GGGSGGGS
XXXQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSXXXXXXXXEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSS

SEQ ID NO: 583 - protein, 251 aa, synthetic construct
ASQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSGGGSEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSS

SEQ ID NO: 584 - protein, 258 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 585 - protein, 253 aa, synthetic construct
ASQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSS

SEQ ID NO: 586 - protein, 258 aa, synthetic construct
  VARIANT 1..3: note = S, GG, AS, ASS or absent
  VARIANT 128..135: note = GGGS, GGGGS, or GGGSGGGS
XXXQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 587 - protein, 253 aa, synthetic construct
ASQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 588 - protein, 256 aa, synthetic construct
  VARIANT 1..3: note = S, GG, AS, ASS or absent
  VARIANT 128..135: note = GGGS, GGGGS, or GGGSGGGS
XXXQVQLLESGGGLVQAGGSLKLSCTNSGRTFSGYGMGWFRQPPGKEREFVSSINWIYGATYYSDSVKGRFALSRDKAKNTVYLQMDSLKPEDTAVYYCAAKINGESYFNTGSFGSWGQGTQVTVSSXXXXXXXXEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSS

SEQ ID NO: 589 - protein, 251 aa, synthetic construct
ASQVQLLESGGGLVQAGGSLKLSCTNSGRTFSGYGMGWFRQPPGKEREFVSSINWIYGATYYSDSVKGRFALSRDKAKNTVYLQMDSLKPEDTAVYYCAAKINGESYFNTGSFGSWGQGTQVTVSSGGGSEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSS

SEQ ID NO: 590 - protein, 253 aa, synthetic construct
  VARIANT 125..132: note = GGGS, GGGGS, or GGGSGGGS
EVQLLESGGGLVQPGGSLRLSCAASGRTFSGYGMGWFRQAPGKEREFVSSINWIYGATYYSDSVKGRFTLSRDKSKNTVYLQMNSLRAEDTAVYYCAAKINGESYFNTGSFGSWGQGTLVTVSSXXXXXXXXEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSS

SEQ ID NO: 591 - protein, 249 aa, synthetic construct
EVQLLESGGGLVQPGGSLRLSCAASGRTFSGYGMGWFRQAPGKEREFVSSINWIYGATYYSDSVKGRFTLSRDKSKNTVYLQMNSLRAEDTAVYYCAAKINGESYFNTGSFGSWGQGTLVTVSSGGGSEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSS

SEQ ID NO: 592 - protein, 488 aa, synthetic construct
  VARIANT 1..3: note = S, GG, AS, ASS or absent
  VARIANT 4: note = E or Q
  VARIANT 16: note = Q or R
  VARIANT 17: note = A or P
  VARIANT 19: note = D or G
  VARIANT 47: note = E or G
  VARIANT 52: note = A or S
  VARIANT 57: note = I or Y
  VARIANT 59: note = D or E
  VARIANT 60: note = A, G or S
  VARIANT 65: note = D or E
  VARIANT 77: note = N or S
  VARIANT 78: note = A or S
  VARIANT 87: note = N or S
  VARIANT 90: note = K or R
  VARIANT 91: note = A or P
  VARIANT 104: note = R or S
  VARIANT 105: note = P or V
  VARIANT 106: note = D or T
  VARIANT 115: note = G or N
  VARIANT 122: note = M or Q
  VARIANT 128..135: note = GGGS, GGGGS, or GGGSGGGS
  VARIANT 136: note = E or Q
  VARIANT 140: note = L or Q
  VARIANT 154: note = K or R
  VARIANT 162: note = I or N
  VARIANT 166: note = E or Y
  VARIANT 167: note = G, P or S
  VARIANT 168: note = V or T
  VARIANT 174: note = Q or R
  VARIANT 179: note = E or G
  VARIANT 180: note = L or R
  VARIANT 185: note = A or C
  VARIANT 189: note = R or S
  VARIANT 191: note = A or G
  VARIANT 197: note = D or N
  VARIANT 198: note = A or S
  VARIANT 210: note = S or T
  VARIANT 220: note = N or S
  VARIANT 222: note = K or R
  VARIANT 223: note = A or P
  VARIANT 226: note = A or T
  VARIANT 215: note = D or Y
  VARIANT 227: note = A or G
  VARIANT 228: note = H or V
  VARIANT 236: note = H or W
  VARIANT 240: note = A or C
  VARIANT 242: note = R or S
  VARIANT 244: note = A or E
  VARIANT 254: note = L, M or Q
XXXXLQLVESGGGLVXXGXSLRLSCAASGRTFSAYGMGWFRQAPGKXREFVXAIRWXGXXTYYAXSVKGRFTISKDXXKNTLYLQMXSLXXEDTAVYYCAAKTXXXSYLSEKSYXYWGQGTXVTVSSXXXXXXXXXVQLXESGGGLVQPGGSLXLSCAASGXSLDXXXIGWFRXAPGKXXEGVSXISAXDXSTHYAXXVKGRFTISRDNXKNTVXLQMNXLXXEDXXXYYCAASLXGLGXKXEXDYDYWGQGTXVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 593 - protein, 488 aa, synthetic construct
  VARIANT 1..3: note = S, GG, AS, ASS or absent
  VARIANT 16: note = Q or R
  VARIANT 17: note = A or P
  VARIANT 47: note = E or G
  VARIANT 52: note = A or S
  VARIANT 57: note = I or Y
  VARIANT 59: note = D or E
  VARIANT 60: note = A, G or S
  VARIANT 65: note = D or E
  VARIANT 78: note = A or S
  VARIANT 87: note = N or S
  VARIANT 90: note = K or R
  VARIANT 91: note = A or P
  VARIANT 104: note = R or S
  VARIANT 105: note = P or V
  VARIANT 106: note = D or T
  VARIANT 115: note = G or N
  VARIANT 122: note = M or Q
  VARIANT 128..135: note = GGGS, GGGGS, or GGGSGGGS
  VARIANT 136: note = E or Q
  VARIANT 179: note = E or G
  VARIANT 180: note = L or R
  VARIANT 185: note = A or C
  VARIANT 191: note = A or G
  VARIANT 198: note = A or S
  VARIANT 210: note = S or T
  VARIANT 215: note = D or Y
  VARIANT 220: note = N or S
  VARIANT 222: note = K or R
  VARIANT 223: note = A or P
  VARIANT 227: note = A or G
  VARIANT 228: note = H or V
  VARIANT 236: note = H or W
  VARIANT 240: note = A or C
  VARIANT 254: note = M or Q
XXXQLQLVESGGGLVXXGGSLRLSCAASGRTFSAYGMGWFRQAPGKXREFVXAIRWXGXXTYYAXSVKGRFTISKDNXKNTLYLQMXSLXXEDTAVYYCAAKTXXXSYLSEKSYXYWGQGTXVTVSSXXXXXXXXXVQLVESGGGLVQPGGSLRLSCAASGNSLDYPTIGWFRQAPGKXXEGVSXISASDXSTHYADXVKGRFTISRDNXKNTVXLQMNXLXXEDTXXYYCAASLXGLGXKSEADYDYWGQGTXVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 594 - protein, 488 aa, synthetic construct
  VARIANT 1..3: note = S, GG, AS, ASS or absent
  VARIANT 128..135: note = GGGS, GGGGS, or GGGSGGGS
XXXQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 595 - protein, 483 aa, synthetic construct
ASQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 596 - protein, 487 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 597 - protein, 483 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 598 - protein, 488 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 599 - protein, 483 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 600 - protein, 488 aa, synthetic construct
  VARIANT 1..3: note = S, GG, AS, ASS or absent
  VARIANT 128..135: note = GGGS, GGGGS, or GGGSGGGS
XXXQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 601 - protein, 483 aa, synthetic construct
ASQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 602 - protein, 488 aa, synthetic construct
  VARIANT 128..135: note = GGGS, GGGGS, or GGGSGGGS
  VARIANT 1..3: note = S, GG, AS, ASS or absent
XXXQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 603 - protein, 483 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 604 - protein, 488 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 605 - protein, 483 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 606 - protein, 487 aa, synthetic construct
  VARIANT 1..3: note = S, GG, AS, ASS or absent
  VARIANT 128..135: note = GGGS, GGGGS, or GGGSGGGS
XXXQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 607 - protein, 482 aa, synthetic construct
ASQLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 608 - protein, 487 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 609 - protein, 482 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 610 - protein, 487 aa, synthetic construct
  VARIANT 1..3: note = S, GG, AS, ASS or absent
  VARIANT 128..135: note = GGGS, GGGGS, or GGGSGGGS
XXXQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 611 - protein, 482 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 612 - protein, 488 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 613 - protein, 483 aa, synthetic construct
ASQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 614 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 615 - protein, 481 aa, synthetic construct
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 616 - protein, 488 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 617 - protein, 483 aa, synthetic construct
ASQVQLQESGGGLVQAGGSLRLSCAASGRTFSSYGMGWFRQAPGKEREFVAAISWIGNSAYYADSVKGRFTLSGDNAKNTLYLQMNSLKPEDTAVYYCAAKYYTDSYYAAASYAYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 618 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 619 - protein, 481 aa, synthetic construct
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 620 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 621 - protein, 481 aa, synthetic construct
EVQLLESGGGLVQPGGSLRLSCAASGRTFSSYGMGWFRQAPGKEREFVAAISWIGNSAYYADSVKGRFTLSGDNSKNTLYLQMNSLRAEDTAVYYCAAKYYTDSYYAAASYAYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 622 - protein, 488 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 623 - protein, 483 aa, synthetic construct
ASQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 624 - protein, 485 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 625 - protein, 481 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 626 - protein, 488 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 627 - protein, 483 aa, synthetic construct
ASQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 628 - protein, 485 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 629 - protein, 481 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 630 - protein, 485 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 631 - protein, 481 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 632 - protein, 487 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 633 - protein, 482 aa, synthetic construct
ASQLQLVESGGGLVQAGGSLRLSCAASGRTFSSYGMGWFRQAPGKERGLVAAINWIPGTTYYADSVKGRFTISGDKDKNTLYLQMDNLKPEDTAVYYCAARRRIDNYYRASEYDYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 634 - protein, 484 aa, synthetic construct
  VARIANT 125..132: note = GGGS, GGGGS, or GGGSGGGS
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 635 - protein, 480 aa, synthetic construct
ELQLVESGGGLVQPGGSLRLSCAASGRTFSSYGMGWFRQAPGKERELVAAINWIPGTTYYADSVKGRFTISGDKSKNTLYLQMNSLRAEDTAVYYCAARRRIDNYYRASEYDYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 636 - protein, 488 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 637 - protein, 483 aa, synthetic construct
ASQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 638 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 639 - protein, 481 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 487 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 641 - protein, 482 aa, synthetic construct
ASQVQLQESGGGLVQAGGSLRLSCAASGRTFSSYGMGWFRQAPGKEREFVAAISWIGGGTYYADSVKGRFTLSRDNAKNMVYLQMNSLKPEDTAVYYCAAKYYPDSYFAAASYAYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 484 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 643 - protein, 480 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 488 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 645 - protein, 483 aa, synthetic construct
ASQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 646 - protein, 485 aa, synthetic construct; identical to SEQ ID NO: 638
  VARIANT 125..132: note = GGGS, GGGGS, or GGGSGGGS

SEQ ID NO: 647 - protein, 481 aa, synthetic construct; identical to SEQ ID NO: 639

SEQ ID NO: 648 - protein, 488 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 649 - protein, 483 aa, synthetic construct
ASQVQLQESGGGLVQAGGSLRLSCAASGRTFSSYGMGWFRQAPGKEREFVAAISWIGGSTYYADSVKGRFTLSSDNAKNRIALQMNSLKPEDTAVYYCAAKYYTDSYFAATSYAYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 650 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 651 - protein, 481 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 652 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 653 - protein, 481 aa, synthetic construct
EVQLLESGGGLVQPGGSLRLSCAASGRTFSSYGMGWFRQAPGKEREFVAAISWIGGGTYYADSVKGRFTLSRDNSKNTVYLQMNSLRAEDTAVYYCAAKYYPDSYFAAASYAYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 654 - protein, 489 aa, synthetic construct
  VARIANT 1..3: note = S, GG, AS, ASS or absent
  VARIANT 129..136: note = GGGS, GGGGS, or GGGSGGGS
XXXQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 655 - protein, 484 aa, synthetic construct
ASQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 656 - protein, 486 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 657 - protein, 482 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 658 - protein, 489 aa, synthetic construct
  VARIANT 1..3: note = S, GG, AS, ASS or absent
  VARIANT 129..136: note = GGGS, GGGGS, or GGGSGGGS
XXXQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 659 - protein, 484 aa, synthetic construct
ASQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 660 - protein, 486 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 661 - protein, 482 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 662 - protein, 486 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 663 - protein, 482 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 664 - protein, 488 aa, synthetic construct
  VARIANT 1..3: note = S, GG, AS, ASS or absent
  VARIANT 129..136: note = GGGS, GGGGS, or GGGSGGGS
XXXQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 665 - protein, 483 aa, synthetic construct
ASQVQLVESGGGLVQAGGSLRLSCAASGRTFSNYAMAWFRQAPGKEREFVSAINWFGRATEYADSVKDRFTTSGDSAKNTVYLQMNSLKPEDTAVYYCAARTKVTTGRRNQAEYDYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 485 aa, synthetic construct
  VARIANT 126..133: note = GGGS, GGGGS, or GGGSGGGS
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 667 - protein, 481 aa, synthetic construct
EVQLVESGGGLVQPGGSLRLSCAASGRTFSNYAMAWFRQAPGKEREFVSAINWFGRATEYADSVKDRFTTSGDSSKNTVYLQMNSLRAEDTAVYYCAARTKVTTGRRNQAEYDYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 668 - protein, 488 aa, synthetic construct
  VARIANT 1..3: note = S, GG, AS, ASS or absent
  VARIANT 128..135: note = GGGS, GGGGS, or GGGSGGGS
XXXQVQLVESGGGSVQAGGSLRLSCAASGRTFSSYGMGWFRQAPGKERGFVATINWIPGNTYYADSVKGRFTISGEEDKNTVYLQMDNLKPEDTAVYYCAARRRIDNYYRASEYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 669 - protein, 483 aa, synthetic construct
ASQVQLVESGGGSVQAGGSLRLSCAASGRTFSSYGMGWFRQAPGKERGFVATINWIPGNTYYADSVKGRFTISGEEDKNTVYLQMDNLKPEDTAVYYCAARRRIDNYYRASEYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 670 - protein, 488 aa, synthetic construct
  VARIANT 1..3: note = S, GG, AS, ASS or absent
  VARIANT 128..135: note = GGGS, GGGGS, or GGGSGGGS
XXXQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 671 - protein, 483 aa, synthetic construct
ASQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 672 - protein, 488 aa, synthetic construct
  VARIANT 1..3: note = S, GG, AS, ASS or absent
  VARIANT 128..135: note = GGGS, GGGGS, or GGGSGGGS
XXXQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 673 - protein, 483 aa, synthetic construct
ASQVQLVESGGGVVQPGGSLRLSCAASGRTFSSYGMGWFRQAPGKEREFVATINWIPGNTYYADSVKGRFTISGDESKNTVYLQMNSLRAEDTAVYYCAARRRIDNYYRASEYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 674 - protein, 488 aa, synthetic construct
  VARIANT 1..3: note = S, GG, AS, ASS or absent
  VARIANT 128..135: note = GGGS, GGGGS, or GGGSGGGS
XXXQVQLVESGGGLVQAGGSLRLSCAASGGTFSYYGMGWFRQAPGKEREFVAAIRWIGGDTYYADSVKGRFTISKDNAKNTLYLQMSSLKPEDTAVYYCAAKTSVTSYLSEKSYNYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 675 - protein, 483 aa, synthetic construct
ASQVQLVESGGGLVQAGGSLRLSCAASGGTFSYYGMGWFRQAPGKEREFVAAIRWIGGDTYYADSVKGRFTISKDNAKNTLYLQMSSLKPEDTAVYYCAAKTSVTSYLSEKSYNYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 676 - protein, 485 aa, synthetic construct
  VARIANT 125..132: note = GGGS, GGGGS, or GGGSGGGS
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 677 - protein, 481 aa, synthetic construct
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 678 - protein, 485 aa, synthetic construct
  VARIANT 125..132: note = GGGS, GGGGS, or GGGSGGGS
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 679 - protein, 481 aa, synthetic construct
EVQLVESGGGLVQPGGSLRLSCAASGGTFSYYGMGWFRQAPGKEREFVAAIRWIGGDTYYADSVKGRFTISKDNSKNTLYLQMNSLRAEDTAVYYCAAKTSVTSYLSEKSYNYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 680 - protein, 488 aa, synthetic construct
  VARIANT 1..3: note = S, GG, AS, ASS or absent
  VARIANT 128..135: note = GGGS, GGGGS, or GGGSGGGS
XXXQVQLQESGGGLVQAGGSLSLSCAASGRTFSSYGMGWFRQAPGKEREFVAAISWIGSATDYADSVRGRFTISGDNAKNTVYLLMNNLKPEDTAVYYCAARRSPMSRTSVLQYDYWGQGTQVTVSSXXXXXXXXQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 681 - protein, 483 aa, synthetic construct
ASQVQLQESGGGLVQAGGSLSLSCAASGRTFSSYGMGWFRQAPGKEREFVAAISWIGSATDYADSVRGRFTISGDNAKNTVYLLMNNLKPEDTAVYYCAARRSPMSRTSVLQYDYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 682 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSXXXXXXXXEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 683 - protein, 481 aa, synthetic construct
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 684 - protein, 485 aa, synthetic construct
  VARIANT 125..132: note = GGGS, GGGGS, or GGGSGGGS
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSXXXXXXXXEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 685 - protein, 481 aa, synthetic construct
EVQLLESGGGLVQPGGSLRLSCAASGRTFSSYGMGWFRQAPGKEREFVAAISWIGSATDYADSVRGRFTISGDNSKNTVYLQMNSLRAEDTAVYYCAARRSPMSRTSVLQYDYWGQGTLVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 686 - protein, 488 aa, synthetic construct
  VARIANT 1..3: note = S, GG, AS, ASS or absent
  VARIANT 128..135: note = GGGS, GGGGS, or GGGSGGGS
XXXQVQLVESGGGLVQAGGSLRLSCAASGRTFSSYGMGWFRQAPGKEREFVAAISWIGNSTYYADSVKGRFTLSGDNAKNTLYLQMNSLKPEDTAVYYCAAKYYTDSYYAAASYAYWGQGTQVTVSSXXXXXXXXQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 687 - protein, 483 aa, synthetic construct
ASQVQLVESGGGLVQAGGSLRLSCAASGRTFSSYGMGWFRQAPGKEREFVAAISWIGNSTYYADSVKGRFTLSGDNAKNTLYLQMNSLKPEDTAVYYCAAKYYTDSYYAAASYAYWGQGTQVTVSSGGGSQVQLQESGGGLVQPGGSLKLSCAASGISLDEGVIGWFRRAPGKEREGVSCISARDGSTHYANAVKGRFTISRDNTKNTVDLQMNNLEPEDAGRYYCAASLWGLGCKREEDYDYWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 688 - protein, 485 aa, synthetic construct
  VARIANT 125..132: note = GGGS, GGGGS, or GGGSGGGS
EVQLVESGGGLVQPGGSLRLSCAASGRTFSSYGMGWFRQAPGKEREFVAAISWIGNSTYYADSVKGRFTLSGDNSKNTLYLQMNSLRAEDTAVYYCAAKYYTDSYYAAASYAYWGQGTLVTVSSXXXXXXXXEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 689 - protein, 481 aa, synthetic construct
EVQLVESGGGLVQPGGSLRLSCAASGRTFSSYGMGWFRQAPGKEREFVAAISWIGNSTYYADSVKGRFTLSGDNSKNTLYLQMNSLRAEDTAVYYCAAKYYTDSYYAAASYAYWGQGTLVTVSSGGGSEVQLLESGGGLVQPGGSLRLSCAASGISLDEGVIGWFRQAPGKEREGVSCISARDGSTHYANAVKGRFTISRDNSKNTVYLQMNSLRAEDTAVYYCAASLWGLGCKREEDYDYWGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 690 - protein, 487 aa, synthetic construct
  VARIANT 2: note = L or V
  VARIANT 5: note = L or V
  VARIANT 14: note = A or P
  VARIANT 24: note = A or N
  VARIANT 44: note = E or G
  VARIANT 49: note = A or E
  VARIANT 69: note = S or T
  VARIANT 74: note = N or S
  VARIANT 75: note = A or S
  VARIANT 82: note = E or Q
  VARIANT 85: note = K or S
  VARIANT 87: note = K or R
  VARIANT 88: note = A or P
  VARIANT 122: note = L or Q
  VARIANT 128..135: note = GGGS, GGGGS, or GGGSGGGS
  VARIANT 136: note = E or Q
  VARIANT 149: note = P or S
  VARIANT 158: note = S or V
  VARIANT 179: note = E or G
  VARIANT 212: note = A or S
  VARIANT 222: note = K or S
  VARIANT 225: note = A or P
  VARIANT 226: note = D or E
  VARIANT 230: note = I or V
  VARIANT 253: note = M or Q
EXQLXESGGGLVQXGGSLRLSCAXSGRTFSSHTVGWFRQAPGKXREFVXAITWNGDTTYFGDFVEGRFXISRDXXKNTLYLXMNXLXXEDTAVYYCAAATGQVWSLQYLRESSYAYWGQGTXVTVSSXXXXXXXXXVQLVESGGGLVQXGGSLRLSCXASGFTSPYSTIGWFRQAPGKXREGVACISSSGRGVNANYIDSVRGRFTISRDNXKNTVYLQMNXLKXXDTAXYYCATNCWRLTSPAADNWGQGTXVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 691 - protein, 487 aa, synthetic construct
  VARIANT 128..135: note = GGGS, GGGGS, or GGGSGGGS
ELQLVESGGGLVQAGGSLRLSCANSGRTFSSHTVGWFRQAPGKEREFVAAITWNGDTTYFGDFVEGRFSISRDSAKNTLYLEMNKLKPEDTAVYYCAAATGQVWSLQYLRESSYAYWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 483 aa, synthetic construct
ELQLVESGGGLVQAGGSLRLSCANSGRTFSSHTVGWFRQAPGKEREFVAAITWNGDTTYFGDFVEGRFSISRDSAKNTLYLEMNKLKPEDTAVYYCAAATGQVWSLQYLRESSYAYWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 693 - protein, 487 aa, synthetic construct
  VARIANT 128..135: note = GGGS, GGGGS, or GGGSGGGS
EVQLLESGGGLVQPGGSLRLSCAASGRTFSSHTVGWFRQAPGKGREFVAAITWNGDTTYFGDFVEGRFTISRDNSKNTLYLQMNSLRAEDTAVYYCAAATGQVWSLQYLRESSYAYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 694 - protein, 483 aa, synthetic construct
EVQLLESGGGLVQPGGSLRLSCAASGRTFSSHTVGWFRQAPGKGREFVAAITWNGDTTYFGDFVEGRFTISRDNSKNTLYLQMNSLRAEDTAVYYCAAATGQVWSLQYLRESSYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 695 - protein, 487 aa, synthetic construct
  VARIANT 128..135: note = GGGS, GGGGS, or GGGSGGGS
ELQLLESGGGLVQPGGSLRLSCAASGRTFSSHTVGWFRQAPGKGREFVSAITWNGDTTYFGDFVEGRFTISRDNSKNTLYLQMNSLRAEDTAVYYCAAATGQVWSLQYLRESSYAYWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 696 - protein, 483 aa, synthetic construct
ELQLLESGGGLVQPGGSLRLSCAASGRTFSSHTVGWFRQAPGKGREFVSAITWNGDTTYFGDFVEGRFTISRDNSKNTLYLQMNSLRAEDTAVYYCAAATGQVWSLQYLRESSYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 697 - protein, 485 aa, synthetic construct
  VARIANT 1..3: note = S, GG, AS, ASS or absent
  VARIANT 128..135: note = GGGS, GGGGS, or GGGSGGGS
XXXQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSXXXXXXXXEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 698 - protein, 480 aa, synthetic construct
ASQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSGGGSEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 699 - protein, 485 aa, synthetic construct
  VARIANT 1..3: note = S, GG, AS, ASS or absent
  VARIANT 128..135: note = GGGS, GGGGS, or GGGSGGGS
XXXQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSXXXXXXXXEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 700 - protein, 480 aa, synthetic construct
ASQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSGGGSEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 701 - protein, 485 aa, synthetic construct
  VARIANT 1..3: note = S, GG, AS, ASS or absent
  VARIANT 128..135: note = GGGS, GGGGS, or GGGSGGGS
XXXQVQLLESGGGLVQAGGSLKLSCTNSGRTFSGYGMGWFRQPPGKEREFVSSINWIYGATYYSDSVKGRFALSRDKAKNTVYLQMDSLKPEDTAVYYCAAKINGESYFNTGSFGSWGQGTQVTVSSXXXXXXXXEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 702 - protein, 480 aa, synthetic construct
ASQVQLLESGGGLVQAGGSLKLSCTNSGRTFSGYGMGWFRQPPGKEREFVSSINWIYGATYYSDSVKGRFALSRDKAKNTVYLQMDSLKPEDTAVYYCAAKINGESYFNTGSFGSWGQGTQVTVSSGGGSEVQLVESGGGLVQAGGSLRLSCAASGSFSSISTMAWYRQVPGKQREWLARIIWSSGATNYADSVKGRFTISRDNTKNTMYLQMDRLTPEDTAAYLCSGAISQTRGTFWTYKSQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 703 - protein, 482 aa, synthetic construct
  VARIANT 125..132: note = GGGS, GGGGS, or GGGSGGGS
EVQLLESGGGLVQPGGSLRLSCAASGRTFSGYGMGWFRQAPGKEREFVSSINWIYGATYYSDSVKGRFTLSRDKSKNTVYLQMNSLRAEDTAVYYCAAKINGESYFNTGSFGSWGQGTLVTVSSXXXXXXXXEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 704 - protein, 478 aa, synthetic construct
EVQLLESGGGLVQPGGSLRLSCAASGRTFSGYGMGWFRQAPGKEREFVSSINWIYGATYYSDSVKGRFTLSRDKSKNTVYLQMNSLRAEDTAVYYCAAKINGESYFNTGSFGSWGQGTLVTVSSGGGSEVQLVESGGGLVQPGGSLRLSCAASGSFSSISTMAWYRQAPGKQREWLARIIWSSGATNYADSVKGRFTISRDNSKNTMYLQMNSLRAEDTAVYYCSGAISQTRGTFWTYKGQGTLVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 705 - protein, 487 aa, synthetic construct
  VARIANT 1..3: note = S, GG, AS, ASS or absent
  VARIANT 128..135: note = S, GG, AS, ASS or absent
XXXQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSXXXXXXXXQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 706 - protein, 482 aa, synthetic construct
ASQLQLVESGGGLVQAGGSLKLSCTASGRTFDGYGMGWFRQAPGKEREFVSAINWIYGATYYADSVKGRFAVSRDKAKGTMYLQMNSLKPEDTAVYYCAAKINAGSYFNIGSFGSWGQGTQVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 707 - protein, 487 aa, synthetic construct
  VARIANT 128..135: note = GGGS, GGGGS, or GGGSGGGS
  VARIANT 1..3: note = S, GG, AS, ASS or absent
XXXQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSXXXXXXXXEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 708 - protein, 482 aa, synthetic construct
ASQLQLVESGGGLVQPGGSLRLSCSASGRTFDGYGMGWFRQAPGKEREFVSAINWIYGATYYADSVKGRFTVSRDKSKNTMYLQMNSLRAEDTAVYYCAAKINAGSYFNIGSFGSWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSSGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 709 - protein, 10 aa, synthetic construct
ASGRTFSSHT

SEQ ID NO: 710 - protein, 127 aa, synthetic construct
  VARIANT 1..3: note = S, GG, AS, ASS or absent
  VARIANT 4: note = E or Q
  VARIANT 8: note = L or Q
  VARIANT 17: note = A or P
  VARIANT 59: note = G or N
  VARIANT 60: note = G or S
  VARIANT 61: note = A or T
  VARIANT 75: note = G, R or S
  VARIANT 78: note = A or S
  VARIANT 81: note = M, R or T
  VARIANT 82: note = I, L or V
  VARIANT 83: note = A or Y
  VARIANT 90: note = K or R
  VARIANT 91: note = A or P
  VARIANT 105: note = P or T
  VARIANT 109: note = F or Y
  VARIANT 112: note = A or T
  VARIANT 122: note = L or Q
XXXXVQLXESGGGLVQXGGSLRLSCAASGRTFSSYGMGWFRQAPGKEREFVAAISWIGXXXYYADSVKGRFTLSXDNXKNXXXLQMNSLXXEDTAVYYCAAKYYXDSYXAAXSYAYWGQGTXVTVSS

SEQ ID NO: 711 - protein, 578 aa, synthetic construct
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 712 - protein, 325 aa, synthetic construct
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 713-714 - skipped (2 entries)

SEQ ID NO: 715 - protein, 4 aa, synthetic construct
GGGS

SEQ ID NO: 716 - protein, 6 aa, synthetic construct
GGSGGS

SEQ ID NO: 717 - protein, 8 aa, synthetic construct
GGGSGGGS

SEQ ID NO: 718 - protein, 9 aa, synthetic construct
GGSGGSGGS

SEQ ID NO: 719 - protein, 214 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 720 - protein, 201 aa, synthetic construct
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 721 - protein, 441 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 722 - protein, 428 aa, synthetic construct
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 723 - protein, 227 aa, synthetic construct
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 724 - protein, 124 aa, synthetic construct
ELQLLESGGGLVQPGGSLRLSCAASGRTFSAYGMGWFRQAPGKEREFVAAIRWIGDSTYYADSVKGRFTISKDNSKNTLYLQMNSLRAEDTAVYYCAAKTSVTSYLSEKSYNYWGQGTMVTVSS

SEQ ID NO: 725 - protein, 124 aa, synthetic construct
ELQLLESGGGLVQPGGSLRLSCAASGRTFSAYGMGWFRQAPGKGREFVSAIRWIGDSTYYADSVKGRFTISKDNSKNTLYLQMNSLRAEDTAVYYCAAKTSVTSYLSEKSYNYWGQGTMVTVSS

SEQ ID NO: 726 - protein, 124 aa, synthetic construct
EVQLVESGGGLVQPGGSLRLSCAASGNSLDYPTIGWFRQAPGKGREGVSCISASDGSTHYADSVKGRFTISRDNSKNTVYLQMNSLRAEDTAVYYCAASLWGLGCKSEADYDYWGQGTQVTVSS

SEQ ID NO: 727 - protein, 124 aa, synthetic construct; identical to SEQ ID NO: 451

SEQ ID NO: 728 - protein, 124 aa, synthetic construct
EVQLVESGGGLVQPGGSLRLSCAASGNSLDYPTIGWFRQAPGKGLEGVSCISASDGSTHYADSVKGRFTISRDNSKNTVYLQMNSLRAEDTAVYYCAASLWGLGCKSEADYDYWGQGTQVTVSS

SEQ ID NO: 729 - protein, 124 aa, synthetic construct
EVQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSS

SEQ ID NO: 730 - protein, 252 aa, synthetic construct
QLQLVESGGGLVRAGGSLRLSCAASGRTFSAYGMGWFRQAPGKEREFVAAIRWIGDSTYYADSVKGRFTISKDNAKNTLYLQMSSLKPEDTAVYYCAAKTSVTSYLSEKSYNYWGQGTQVTVSSGGGSQVQLVESGGGLVQPGGSLRLSCAASGNSLDYPTIGWFRQAPGKEREGVSCISASDGSTHYADSVKGRFTISRDNTKNTVDLQMNNLKPEDTGHYYCAASLWGLGCKSEADYDYWGQGTQVTVSS

SEQ ID NO: 731 - protein, 254 aa, synthetic construct; identical to SEQ ID NO: 478

SEQ ID NO: 732 - protein, 252 aa, synthetic construct
EVQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSCISASDGSTHYADSVKGRFTISRDNSKNTVYLQMNSLRAEDTAVYYCAASLWGLGCKSEADYDYWGQGTQVTVSS

SEQ ID NO: 733 - protein, 254 aa, synthetic construct
ASEVQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSCISASDGSTHYADSVKGRFTISRDNSKNTVYLQMNSLRAEDTAVYYCAASLWGLGCKSEADYDYWGQGTQVTVSS

SEQ ID NO: 734 - protein, 254 aa, synthetic construct
ASQVQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSCISASDGSTHYADSVKGRFTISRDNSKNTVYLQMNSLRAEDTAVYYCAASLWGLGCKSEADYDYWGQGTQVTVSS

SEQ ID NO: 735 - protein, 254 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSCISASDGSTHYADSVKGRFTISRDNSKNTVYLQMNSLRAEDTAVYYCAASLWGLGCKSEADYDYWGQGTQVTVSS

SEQ ID NO: 736 - protein, 252 aa, synthetic construct
GLQLVESGGGLVQPGGSLRLSCAASGRTFSAYGMGWFRQAPGKGREFVAAIRWIGDSTYYADSVKGRFTISKDNSKNTLYLQMNSLRAEDTAVYYCAAKTSVTSYLSEKSYNYWGQGTLVTVSSGGGSEVQLVESGGGLVQPGGSLRLSCAASGNSLDYPTIGWFRQAPGKGLEGVSCISASDGSTHYADSVKGRFTISRDNSKNTVYLQMNSLRAEDTAVYYCAASLWGLGCKSEADYDYWGQGTQVTVSS

SEQ ID NO: 737 - protein, 254 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 738 - protein, 254 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASGHGLGCKSEADYDYWGQGTMVTVSS

SEQ ID NO: 739 - protein, 254 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASLHGAGCKSEADYDYWGQGTMVTVSS

SEQ ID NO: 740 - protein, 254 aa, synthetic construct; identical to SEQ ID NO: 482

SEQ ID NO: 741 - protein, 254 aa, synthetic construct
ASQLQLLESGGGLVQPGGSLRLSCAASGRTFSAYGMGWFRQAPGKGREFVSAIRWIGDATYYAESVKGRFTISKDNSKNTLYLQMNSLRAEDTAVYYCAAKTSVTSYLSEKSYNYWGQGTMVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 742 - protein, 254 aa, synthetic construct
ASQLQLLESGGGLVQPGGSLRLSCAASGRTFSAYGMGWFRQAPGKGREFVSAIRWFGEGTYYAESVKGRFTISKDNSKNTLYLQMNSLRAEDTAVYYCAAKTSPTSYLSEKSYGYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 743 - protein, 254 aa, synthetic construct
ASQLQLLESGGGLVQPGGSLRLSCAASGRTFSAYGMGWFRQAPGKGREFVSAIRWFGEGTYYAESVKGRFTISKDNSKNTLYLQMNSLRAEDTAVYYCAAKTSPTSYLSEKSYGYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASGHGLGCKSEADYDYWGQGTMVTVSS

SEQ ID NO: 744 - protein, 254 aa, synthetic construct
ASQLQLLESGGGLVQPGGSLRLSCAASGRTFSAYGMGWFRQAPGKGREFVSAIRWFGEGTYYAESVKGRFTISKDNSKNTLYLQMNSLRAEDTAVYYCAAKTSPTSYLSEKSYGYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASLHGAGCKSEADYDYWGQGTMVTVSS

SEQ ID NO: 745 - protein, 254 aa, synthetic construct
ASQLQLLESGGGLVQPGGSLRLSCAASGRTFSAYGMGWFRQAPGKGREFVSAIRWFGEGTYYAESVKGRFTISKDNSKNTLYLQMNSLRAEDTAVYYCAAKTSPTSYLSEKSYGYWGQGTMVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 746 - protein, 254 aa, synthetic construct
ASQLQLLESGGGLVQPGGSLRLSCAASGRTFSAYGMGWFRQAPGKGREFVSAIRWFGEGTYYAESVKGRFTISKDNSKNTLYLQMNSLRAEDTAVYYCAAKTSPTSYLSEKSYGYWGQGTMVTVSSGGGSEVQLVESGGGLVQPGGSLRLSCAASGNSLDYPTIGWFRQAPGKGLEGVSAISASDASTHYADAVKGRFTISRDNSKNTVYLQMNSLRAEDTAVYYCAASLHGLGAKSEADYDYWGQGTMVTVSS

SEQ ID NO: 747 - protein, 254 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 748 - protein, 254 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLVESGGGLVQPGGSLRLSCAASGNSLDYPTIGWFRQAPGKGLEGVSCISASEGSTHYADAVKGRFTISRDNSKNTVYLQMNSLRAEDTAVYYCAASGHGLGCKSEADYDYWGQGTMVTVSS

SEQ ID NO: 749 - protein, 254 aa, synthetic construct; identical to SEQ ID NO: 748

SEQ ID NO: 750 - protein, 254 aa, synthetic construct
ASQLQLLESGGGLVQPGGSLRLSCAASGRTFSAYGMGWFRQAPGKGREFVSAIRWYGEGTYYAESVKGRFTISKDNSKNTLYLQMNSLRAEDTAVYYCAAKTRPDSYLSEKSYGYWGQGTMVTVSSGGGSEVQLVESGGGLVQPGGSLRLSCAASGNSLDYSTIGWFRQAPGKGLEGVSCISASEGSTHYADAVKGRFTISRDNSKNTVYLQMNSLRAEDTAVYYCAASLHGLGCKSEADYDYWGQGTMVTVSS

SEQ ID NO: 751 - protein, 254 aa, synthetic construct; identical to SEQ ID NO: 480

SEQ ID NO: 752 - protein, 127 aa, synthetic construct
EVQLLESGGGLVQPGGSLRLSCANSGRTFSSHTVGWFRQAPGKGREFVSAITWNGDTTYFGDFVEGRFTISRDNSKNTLYLQMNSLRAEDTAVYYCAAATGQVWSLQYLRESSYAYWGQGTLVTVSS

SEQ ID NO: 753 - protein, 127 aa, synthetic construct
EVQLLESGGGLVQPGGSLRLSCAASGRTFSSHTVGWFRQAPGKGREFVSAITWNGDTTYFGDFVEGRFTISRDSSKNTLYLQMNSLRAEDTAVYYCAAATGQVWSLQYLRESSYAYWGQGTLVTVSS

SEQ ID NO: 754 - protein, 127 aa, synthetic construct; identical to SEQ ID NO: 251

SEQ ID NO: 755 - protein, 127 aa, synthetic construct; identical to SEQ ID NO: 252

SEQ ID NO: 756 - protein, 254 aa, synthetic construct; identical to SEQ ID NO: 575

SEQ ID NO: 757 - protein, 254 aa, synthetic construct
EVQLLESGGGLVQPGGSLRLSCAASGRTFSSHTVGWFRQAPGKGREFVSAITWNGDTTYFGDFVEGRFTISRDNSKNTLYLQMNSLRAEDTAVYYCAAATGQVWSLQYLRESSYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 758 - protein, 254 aa, synthetic construct
ELQLLESGGGLVQPGGSLRLSCAASGRTFSSHTVGWFRQAPGKGREFVAAITWNGDTTYFGDFVEGRFTISRDSSKNTLYLQMNSLRAEDTAVYYCAAATGQVWSLQYLRESSYAYWGQGTLVTVSSGGGSEVQLVESGGGLVQPGGSLRLSCSASGFTSPYSTIGWFRQAPGKGREGVACISSSGRGVNANYIDSVRGRFTISRDNSKNTVYLQMNSLRAEDTAVYYCATNCWRLTSPAADNWGQGTMVTVSS

SEQ ID NO: 759 - protein, 258 aa, synthetic construct
ELQLVESGGGLVQPGGSLRLSCAASGRTFSAYGMGWFRQAPGKGREFVAAIRWIGDSTYYADSVKGRFTISKDNSKNTLYLQMNSLRPEDTAVYYCAAKTSVTSYLSEKSYNYWGKGTPVTVSSGGGSEVQLVESGGGLVQPGGSLRLSCAASGNSLDYPTIGWFRQAPGKGREGVSCISASDGSTHYADSVKGRFTISRDNSKNTLYLQMNSLRPEDTAVYYCAASLWGLGCKSEADYDYWGKGTPVTVSSHHHHHH

SEQ ID NO: 760 - protein, 260 aa, synthetic construct
ELQLVESGGGLVQAGGSLRLSCANSGRTFSSHTVGWFRQAPGKEREFVAAITWNGDTTYFGDFVEGRFSISRDSAKNTLYLEMNKLKPEDTAVYYCAAATGQVWSLQYLRESSYAYWGKGTPVTVSSGGGSQVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSHHHHHH

SEQ ID NO: 761 - protein, 130 aa, synthetic construct
QRQLVESGGGLVQPGGSLRLSCAASGNSLDYPTIGWFRQAPGKEREGVSCISASDGSTHYADSVKGRFTISRDNTKNTVYLQMNSLEPEDTAVYYCAASLWGLGCKSEADYDYWGKGTPVTVSSHHHHHH

SEQ ID NO: 762 - protein, 129 aa, synthetic construct
QVQLVESGGGLVQSGGSLRLSCVASGFTSPYSTIGWFRQAPGKEREGVACISSSGRGVNANYIDSVRGRFTISRDNAKNTVYLQMNKLKPDDTAIYYCATNCWRLTSPAADNWGQGTQVTVSSHHHHHH

SEQ ID NO: 763 - protein, 13 aa, synthetic construct
PKYVKQNTLKLAT

SEQ ID NO: 764 - DNA, 378 bp, synthetic construct
  source 1..378: mol_type = other DNA
gcaagtcaactacaactactagaaagtggtggcggactggtccaacccggcggctccctgagactgtcttgcgccgcttctggccggaccttctccgcctacggcatgggctggttcagacaggcccctggcaagggcagagagttcgtgagcgccatcagatggtacggcgagggcacctactacgccgagtccgtgaaaggcagattcaccatcagcaaggacaactccaaaaacaccctgtacctgcagatgaactccctgcgggctgaggacaccgctgtgtactactgtgctgctaagacccggcctgactcctacctgagcgagaagtcttatggctattggggccagggcacaatggtcacagtgtcctcc

SEQ ID NO: 765 - DNA, 378 bp, synthetic construct
  source 1..378: mol_type = other DNA
gcaagtcaactacaactactagaaagtggtggaggcctcgtccaacccggcggcagcctgcggctgagctgtgctgcctctggccggaccttctccgcctacggcatgggctggttccggcaggctcctggcaaaggcagagagttcgtgagcgccatcagatggatcggcgacgctacctactacgctgagtccgtgaagggccggttcaccatctccaaggacaactccaagaacaccctgtacctgcagatgaattctctgcgcgccgaggacaccgccgtgtactactgtgccgccaagacctctgttacctcctacctgtctgagaagtcttacaactactggggccagggcaccatggtgacagtgtcctct

SEQ ID NO: 766 - DNA, 378 bp, synthetic construct
  source 1..378: mol_type = other DNA
gcctctcagctccagctgttggaatctggcggaggattggttcagcctggcggctctctgagactgtcttgtgctgcttccggcagaaccttcagcgcttatggcatgggctggttcagacaggctcctggcaagggcagagaattcgtgtccgccatcagatggatcggcgacgccacctactacgccgagtctgtgaagggcagattcaccatctccaaggacaactccaagaacaccctgtacctgcagatgaactccctgagagccgaggacaccgccgtgtactactgtgccgctaagacctccgtgacctcctacctgtccgagaagtcctacaactactggggccagggcaccatggtcaccgtttctagc

SEQ ID NO: 767 - DNA, 381 bp, synthetic construct
  source 1..381: mol_type = other DNA
gaagtgcagctactggagagcggcggtggcctggtccagccaggagggagcttgagattgagttgcgctgcctcaggtcgaacattcagttctcacaccgtgggatggttccggcaagcccctggtaagggaagagagttcgtcgctgccataacttggaacggggataccacatacttcggggattttgtcgagggaaggttcacaatttctagggataactccaagaacacactgtatcttcagatgaactctctccgggctgaagataccgctgtatactattgcgctgcagccactggtcaagtgtggagcctccagtatcttcgagaaagtagctacgcttactggggccagggcaccctggtcacagtgtcttcc

SEQ ID NO: 768 - DNA, 381 bp, synthetic construct
  source 1..381: mol_type = other DNA
gagctacagctgctcgagtctggaggcgggctggtacagcccggtgggagcctcaggctgagctgcgctgcaagcgggagaacgttcagcagtcatactgtgggctggttcaggcaggcacccgggaagggtcgagaattcgtgtccgctatcacctggaatggggacacaacctactttggtgatttcgtggaggggcgattcacaatctcaagggataactccaagaacaccctgtacctacagatgaactcccttagggcagaagatacagctgtgtactattgcgctgcagcgaccggtcaggtgtggtcactccagtacctgagagaatccagttatgcctactggggtcagggcaccctagtcacagtgtcttcc

SEQ ID NO: 769 - DNA, 381 bp, synthetic construct
  source 1..381: mol_type = other DNA
gaactgcagctgttggaatctggcggcggattggttcagcctggcggatctctgagactgtcttgtgccgcttccggcagaaccttctcctctcataccgtcggctggttcagacaggctcctggcaagggcagagaattcgtgtctgccatcacctggaacggcgacaccacctacttcggcgatttcgtggaaggccggttcaccatctctcgggacaactccaagaacaccctgtacctgcagatgaactccctgagagccgaggacaccgccgtgtactattgtgctgctgctaccggccaagtgtggtccctgcagtatctgagagagtcctcctacgcctattggggccagggcacactggtcacagtttctagt

SEQ ID NO: 770 - DNA, 372 bp, synthetic construct
  source 1..372: mol_type = other DNA
gaagtgcagctggtggaatccggcggaggcctcgtgcagcctggaggctctctgcggctgtcctgcgccgcctctggcaactccctggactaccctaccatcggctggttccggcaggctccaggcaagggcctggaaggcgtgtctgctatctctgcctccgacgcctctacccactacgccgacgctgtgaagggaaggtttacaatctccagagataattctaagaacaccgtgtacctgcagatgaactctctgagagccgaggataccgccgtgtactactgcgctgctagcctgcacggcctgggcgccaagtccgaggccgattacgactactggggccagggcaccatggtgaccgtgtccagc

SEQ ID NO: 771 - DNA, 372 bp, synthetic construct
  source 1..372: mol_type = other DNA
gaagtgcagctggtggaatctggcggcggactggtgcagcctggaggatctctgagactgtcctgcgctgcttccggcaactccctggactactccacaatcggctggtttagacaggcccctggcaagggcctggaaggcgtgtcttgcatctctgcctccgagggctctacccactacgccgacgccgtgaaaggcagattcaccatcagcagagacaacagcaagaacaccgtgtatctgcagatgaactccctgagagctgaggatacagctgtgtactattgcgccgcttctctgcacggcctgggctgcaagtccgaggccgattacgattactggggacagggcaccatggtcaccgtgtcctcc

SEQ ID NO: 772 - DNA, 372 bp, synthetic construct
  source 1..372: mol_type = other DNA
gaggtgcagctggttgaaagtggtggcggacttgtgcaacctggcggaagtctgagactcagctgtgccgcctctggcaactccctggactattctaccatcggctggtttaggcaggcacccggaaaaggactggaaggcgtgtcctgtatctccgcctctgagggctctacccactacgctgatgccgtgaaaggccggttcaccatcagccgggacaacagcaagaataccgtctatctccagatgaacagcctgcgcgctgaggatacagctgtgtactattgcgccgcctctctgcatggcctgggctgcaaatctgaggccgactacgattattggggacagggaacaatggttacagtgtcctcc

SEQ ID NO: 773 - DNA, 369 bp, synthetic construct
  source 1..369: mol_type = other DNA
gaggtgcagctggtggagtctggaggtggcttggtgcagccgggcggatccctacgcctctcatgtagcgcttccggatttacctccccttatagcaccatagggtggttccggcaggctccagggaaaggacgcgaaggggtggcctgtatcagctcttccggccgaggcgtgaatgcgaattacatcgactcagtgaggggtcgcttcaccatcagccgggacaacagcaagaacacagtttacctccaaatgaacagcctgagggccgaggacacagctgtgtattactgtgctacaaactgctggcgccttacaagtcccgcagccgataattggggtcagggaactatggtcacagtgtcttcc

SEQ ID NO: 774 - DNA, 369 bp, synthetic construct; identical to SEQ ID NO: 773
  source 1..369: mol_type = other DNA

SEQ ID NO: 775 - DNA, 369 bp, synthetic construct
  source 1..369: mol_type = other DNA
gaggtgcagctggttgaaagcggcggaggacttgttcaaccaggcggaagcctgcggctgtcttgttctgcctctggcttcacctctccttactccaccatcggatggttccggcaggcccctggaaagggacgagaaggtgtcgcctgcatctccagctctggaagaggcgtgaacgccaactacatcgactctgtgcggggcagattcaccatcagcagagacaacagcaagaataccgtgtatctccaaatgaacagcctgcgcgctgaggataccgctgtgtattactgcgccaccaactgctggcggctgacctctcctgctgctgataattggggacagggaactatggttaccgtgtcctcc

SEQ ID NO: 776 - DNA, 762 bp, synthetic construct
  source 1..762: mol_type = other DNA
gcaagtcaactacaactactagaaagtggtggcggactggtccaacccggcggctccctgagactgtcttgcgccgcttctggccggaccttctccgcctacggcatgggctggttcagacaggcccctggcaagggcagagagttcgtgagcgccatcagatggtacggcgagggcacctactacgccgagtccgtgaaaggcagattcaccatcagcaaggacaactccaaaaacaccctgtacctgcagatgaactccctgcgggctgaggacaccgctgtgtactactgtgctgctaagacccggcctgactcctacctgagcgagaagtcttatggctattggggccagggcacaatggtcacagtgtcctccggcggaggatctgaagtgcagctggtggaatccggcggaggcctcgtgcagcctggaggctctctgcggctgtcctgcgccgcctctggcaactccctggactaccctaccatcggctggttccggcaggctccaggcaagggcctggaaggcgtgtctgctatctctgcctccgacgcctctacccactacgccgacgctgtgaagggaaggtttacaatctccagagataattctaagaacaccgtgtacctgcagatgaactctctgagagccgaggataccgccgtgtactactgcgctgctagcctgcacggcctgggcgccaagtccgaggccgattacgactactggggccagggcaccatggtgaccgtgtccagc

SEQ ID NO: 777 - DNA, 762 bp, synthetic construct
  source 1..762: mol_type = other DNA
gcaagtcaactacaactactagaaagtggtggaggcctcgtccaacccggcggcagcctgcggctgagctgtgctgcctctggccggaccttctccgcctacggcatgggctggttccggcaggctcctggcaaaggcagagagttcgtgagcgccatcagatggatcggcgacgctacctactacgctgagtccgtgaagggccggttcaccatctccaaggacaactccaagaacaccctgtacctgcagatgaattctctgcgcgccgaggacaccgccgtgtactactgtgccgccaagacctctgttacctcctacctgtctgagaagtcttacaactactggggccagggcaccatggtgacagtgtcctctggtggcggcagcgaagtgcagctggtggaatctggcggcggactggtgcagcctggaggatctctgagactgtcctgcgctgcttccggcaactccctggactactccacaatcggctggtttagacaggcccctggcaagggcctggaaggcgtgtcttgcatctctgcctccgagggctctacccactacgccgacgccgtgaaaggcagattcaccatcagcagagacaacagcaagaacaccgtgtatctgcagatgaactccctgagagctgaggatacagctgtgtactattgcgccgcttctctgcacggcctgggctgcaagtccgaggccgattacgattactggggacagggcaccatggtcaccgtgtcctcc

SEQ ID NO: 778 - DNA, 762 bp, synthetic construct
  source 1..762: mol_type = other DNA
gcctctcagctccagctgttggaatctggcggaggattggttcagcctggcggctctctgagactgtcttgtgctgcttccggcagaaccttcagcgcttatggcatgggctggttcagacaggctcctggcaagggcagagaattcgtgtccgccatcagatggatcggcgacgccacctactacgccgagtctgtgaagggcagattcaccatctccaaggacaactccaagaacaccctgtacctgcagatgaactccctgagagccgaggacaccgccgtgtactactgtgccgctaagacctccgtgacctcctacctgtccgagaagtcctacaactactggggccagggcaccatggtcaccgtttctagcggaggcggatctgaggtgcagctggttgaaagtggtggcggacttgtgcaacctggcggaagtctgagactcagctgtgccgcctctggcaactccctggactattctaccatcggctggtttaggcaggcacccggaaaaggactggaaggcgtgtcctgtatctccgcctctgagggctctacccactacgctgatgccgtgaaaggccggttcaccatcagccgggacaacagcaagaataccgtctatctccagatgaacagcctgcgcgctgaggatacagctgtgtactattgcgccgcctctctgcatggcctgggctgcaaatctgaggccgactacgattattggggacagggaacaatggttacagtgtcctcc

SEQ ID NO: 779 - DNA, 762 bp, synthetic construct
  source 1..762: mol_type = other DNA
gaagtgcagctactggagagcggcggtggcctggtccagccaggagggagcttgagattgagttgcgctgcctcaggtcgaacattcagttctcacaccgtgggatggttccggcaagcccctggtaagggaagagagttcgtcgctgccataacttggaacggggataccacatacttcggggattttgtcgagggaaggttcacaatttctagggataactccaagaacacactgtatcttcagatgaactctctccgggctgaagataccgctgtatactattgcgctgcagccactggtcaagtgtggagcctccagtatcttcgagaaagtagctacgcttactggggccagggcaccctggtcacagtgtcttccggaggcggaagcgaggtgcagctggtggagtctggaggtggcttggtgcagccgggcggatccctacgcctctcatgtagcgcttccggatttacctccccttatagcaccatagggtggttccggcaggctccagggaaaggacgcgaaggggtggcctgtatcagctcttccggccgaggcgtgaatgcgaattacatcgactcagtgaggggtcgcttcaccatcagccgggacaacagcaagaacacagtttacctccaaatgaacagcctgagggccgaggacacagctgtgtattactgtgctacaaactgctggcgccttacaagtcccgcagccgataattggggtcagggaactatggtcacagtgtcttcc

SEQ ID NO: 780 - DNA, 762 bp, synthetic construct
  source 1..762: mol_type = other DNA
gagctacagctgctcgagtctggaggcgggctggtacagcccggtgggagcctcaggctgagctgcgctgcaagcgggagaacgttcagcagtcatactgtgggctggttcaggcaggcacccgggaagggtcgagaattcgtgtccgctatcacctggaatggggacacaacctactttggtgatttcgtggaggggcgattcacaatctcaagggataactccaagaacaccctgtacctacagatgaactcccttagggcagaagatacagctgtgtactattgcgctgcagcgaccggtcaggtgtggtcactccagtacctgagagaatccagttatgcctactggggtcagggcaccctagtcacagtgtcttccggaggcggaagcgaggtgcagctggtggagtctggaggtggcttggtgcagccgggcggatccctacgcctctcatgtagcgcttccggatttacctccccttatagcaccatagggtggttccggcaggctccagggaaaggacgcgaaggggtggcctgtatcagctcttccggccgaggcgtgaatgcgaattacatcgactcagtgaggggtcgcttcaccatcagccgggacaacagcaagaacacagtttacctccaaatgaacagcctgagggccgaggacacagctgtgtattactgtgctacaaactgctggcgccttacaagtcccgcagccgataattggggtcagggaactatggtcacagtgtcttcc

SEQ ID NO: 781 - DNA, 762 bp, synthetic construct
  source 1..762: mol_type = other DNA
gaactgcagctgttggaatctggcggcggattggttcagcctggcggatctctgagactgtcttgtgccgcttccggcagaaccttctcctctcataccgtcggctggttcagacaggctcctggcaagggcagagaattcgtgtctgccatcacctggaacggcgacaccacctacttcggcgatttcgtggaaggccggttcaccatctctcgggacaactccaagaacaccctgtacctgcagatgaactccctgagagccgaggacaccgccgtgtactattgtgctgctgctaccggccaagtgtggtccctgcagtatctgagagagtcctcctacgcctattggggccagggcacactggtcacagtttctagtggcggaggctccgaggtgcagctggttgaaagcggcggaggacttgttcaaccaggcggaagcctgcggctgtcttgttctgcctctggcttcacctctccttactccaccatcggatggttccggcaggcccctggaaagggacgagaaggtgtcgcctgcatctccagctctggaagaggcgtgaacgccaactacatcgactctgtgcggggcagattcaccatcagcagagacaacagcaagaataccgtgtatctccaaatgaacagcctgcgcgctgaggataccgctgtgtattactgcgccaccaactgctggcggctgacctctcctgctgctgataattggggacagggaactatggttaccgtgtcctcc

SEQ ID NO: 782 - DNA, 1449 bp, synthetic construct
  source 1..1449: mol_type = other DNA
gcaagtcaactacaactactagaaagtggtggcggactggtccaacccggcggctccctgagactgtcttgcgccgcttctggccggaccttctccgcctacggcatgggctggttcagacaggcccctggcaagggcagagagttcgtgagcgccatcagatggtacggcgagggcacctactacgccgagtccgtgaaaggcagattcaccatcagcaaggacaactccaaaaacaccctgtacctgcagatgaactccctgcgggctgaggacaccgctgtgtactactgtgctgctaagacccggcctgactcctacctgagcgagaagtcttatggctattggggccagggcacaatggtcacagtgtcctccggcggaggatctgaagtgcagctggtggaatccggcggaggcctcgtgcagcctggaggctctctgcggctgtcctgcgccgcctctggcaactccctggactaccctaccatcggctggttccggcaggctccaggcaagggcctggaaggcgtgtctgctatctctgcctccgacgcctctacccactacgccgacgctgtgaagggaaggtttacaatctccagagataattctaagaacaccgtgtacctgcagatgaactctctgagagccgaggataccgccgtgtactactgcgctgctagcctgcacggcctgggcgccaagtccgaggccgattacgactactggggccagggcaccatggtgaccgtgtccagcggttctgacaagacccacacctgtcctccatgtcctgctccagaagctgctggcgccccatccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaa

SEQ ID NO: 783 - DNA, 1449 bp, synthetic construct
  source 1..1449: mol_type = other DNA
gcaagtcaactacaactactagaaagtggtggaggcctcgtccaacccggcggcagcctgcggctgagctgtgctgcctctggccggaccttctccgcctacggcatgggctggttccggcaggctcctggcaaaggcagagagttcgtgagcgccatcagatggatcggcgacgctacctactacgctgagtccgtgaagggccggttcaccatctccaaggacaactccaagaacaccctgtacctgcagatgaattctctgcgcgccgaggacaccgccgtgtactactgtgccgccaagacctctgttacctcctacctgtctgagaagtcttacaactactggggccagggcaccatggtgacagtgtcctctggtggcggcagcgaagtgcagctggtggaatctggcggcggactggtgcagcctggaggatctctgagactgtcctgcgctgcttccggcaactccctggactactccacaatcggctggtttagacaggcccctggcaagggcctggaaggcgtgtcttgcatctctgcctccgagggctctacccactacgccgacgccgtgaaaggcagattcaccatcagcagagacaacagcaagaacaccgtgtatctgcagatgaactccctgagagctgaggatacagctgtgtactattgcgccgcttctctgcacggcctgggctgcaagtccgaggccgattacgattactggggacagggcaccatggtcaccgtgtcctccggctccgacaagacccacacctgtcctccatgtcctgctccagaagctgctggcgccccatccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaa

SEQ ID NO: 784 - DNA, 1449 bp, synthetic construct
  source 1..1449: mol_type = other DNA
gcctctcagctccagctgttggaatctggcggaggattggttcagcctggcggctctctgagactgtcttgtgctgcttccggcagaaccttcagcgcttatggcatgggctggttcagacaggctcctggcaagggcagagaattcgtgtccgccatcagatggatcggcgacgccacctactacgccgagtctgtgaagggcagattcaccatctccaaggacaactccaagaacaccctgtacctgcagatgaactccctgagagccgaggacaccgccgtgtactactgtgccgctaagacctccgtgacctcctacctgtccgagaagtcctacaactactggggccagggcaccatggtcaccgtttctagcggaggcggatctgaggtgcagctggttgaaagtggtggcggacttgtgcaacctggcggaagtctgagactcagctgtgccgcctctggcaactccctggactattctaccatcggctggtttaggcaggcacccggaaaaggactggaaggcgtgtcctgtatctccgcctctgagggctctacccactacgctgatgccgtgaaaggccggttcaccatcagccgggacaacagcaagaataccgtctatctccagatgaacagcctgcgcgctgaggatacagctgtgtactattgcgccgcctctctgcatggcctgggctgcaaatctgaggccgactacgattattggggacagggaacaatggttacagtgtcctccggcagcgacaagacccacacctgtcctccatgtcctgctccagaagctgctggcgccccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatcagcaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaag

SEQ ID NO: 785 - DNA, 1449 bp, synthetic construct
  source 1..1449: mol_type = other DNA
gaagtgcagctactggagagcggcggtggcctggtccagccaggagggagcttgagattgagttgcgctgcctcaggtcgaacattcagttctcacaccgtgggatggttccggcaagcccctggtaagggaagagagttcgtcgctgccataacttggaacggggataccacatacttcggggattttgtcgagggaaggttcacaatttctagggataactccaagaacacactgtatcttcagatgaactctctccgggctgaagataccgctgtatactattgcgctgcagccactggtcaagtgtggagcctccagtatcttcgagaaagtagctacgcttactggggccagggcaccctggtcacagtgtcttccggaggcggaagcgaggtgcagctggtggagtctggaggtggcttggtgcagccgggcggatccctacgcctctcatgtagcgcttccggatttacctccccttatagcaccatagggtggttccggcaggctccagggaaaggacgcgaaggggtggcctgtatcagctcttccggccgaggcgtgaatgcgaattacatcgactcagtgaggggtcgcttcaccatcagccgggacaacagcaagaacacagtttacctccaaatgaacagcctgagggccgaggacacagctgtgtattactgtgctacaaactgctggcgccttacaagtcccgcagccgataattggggtcagggaactatggtcacagtgtcttccggatcagacaagacccacacctgtcctccatgtcctgctccagaagctgctggcgccccatccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaa

SEQ ID NO: 786 - DNA, 1449 bp, synthetic construct
  source 1..1449: mol_type = other DNA
gagctacagctgctcgagtctggaggcgggctggtacagcccggtgggagcctcaggctgagctgcgctgcaagcgggagaacgttcagcagtcatactgtgggctggttcaggcaggcacccgggaagggtcgagaattcgtgtccgctatcacctggaatggggacacaacctactttggtgatttcgtggaggggcgattcacaatctcaagggataactccaagaacaccctgtacctacagatgaactcccttagggcagaagatacagctgtgtactattgcgctgcagcgaccggtcaggtgtggtcactccagtacctgagagaatccagttatgcctactggggtcagggcaccctagtcacagtgtcttccggaggcggaagcgaggtgcagctggtggagtctggaggtggcttggtgcagccgggcggatccctacgcctctcatgtagcgcttccggatttacctccccttatagcaccatagggtggttccggcaggctccagggaaaggacgcgaaggggtggcctgtatcagctcttccggccgaggcgtgaatgcgaattacatcgactcagtgaggggtcgcttcaccatcagccgggacaacagcaagaacacagtttacctccaaatgaacagcctgagggccgaggacacagctgtgtattactgtgctacaaactgctggcgccttacaagtcccgcagccgataattggggtcagggaactatggtcacagtgtcttccggatcagacaagacccacacctgtcctccatgtcctgctccagaagctgctggcgccccatccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggaaaa

SEQ ID NO: 787 - DNA, 1449 bp, synthetic construct
  source 1..1449: mol_type = other DNA
gaactgcagctgttggaatctggcggcggattggttcagcctggcggatctctgagactgtcttgtgccgcttccggcagaaccttctcctctcataccgtcggctggttcagacaggctcctggcaagggcagagaattcgtgtctgccatcacctggaacggcgacaccacctacttcggcgatttcgtggaaggccggttcaccatctctcgggacaactccaagaacaccctgtacctgcagatgaactccctgagagccgaggacaccgccgtgtactattgtgctgctgctaccggccaagtgtggtccctgcagtatctgagagagtcctcctacgcctattggggccagggcacactggtcacagtttctagtggcggaggctccgaggtgcagctggttgaaagcggcggaggacttgttcaaccaggcggaagcctgcggctgtcttgttctgcctctggcttcacctctccttactccaccatcggatggttccggcaggcccctggaaagggacgagaaggtgtcgcctgcatctccagctctggaagaggcgtgaacgccaactacatcgactctgtgcggggcagattcaccatcagcagagacaacagcaagaataccgtgtatctccaaatgaacagcctgcgcgctgaggataccgctgtgtattactgcgccaccaactgctggcggctgacctctcctgctgctgataattggggacagggaactatggttaccgtgtcctccggcagcgacaagacccatacctgtcctccatgtcctgctccagaagctgctggcgccccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctggtcaagggcttctacccttccgatatcgccgtggaatgggagagcaatggccagcctgagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaag

SEQ ID NO: 788 - protein, 487 aa, synthetic construct
QVQLQESGGGSVQAGGSLRLSCAASGYSNCSYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVQAGGSLRLSCAASGYSYSSYCMGWFRQAPGKEREGVATIDSDGMTRYADSVKGRFTISKDNAKNTLYLQMNSLKPEDTAMYYCAAPLYDCDSGAVGRNPPYWGQGTQVTVSSASRVESKYGPPCPPCPAPEFLGGPSVFLFPPKPKDTLMISRTPEVTCVVVDVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 789 - protein, 258 aa, synthetic construct
DEVQLQESGGGSVQAGGSLRLSCAASGYSNCAYDMTWYRQAPGKEREFVSAIHSDGSTRYADSVKGRFFISQDNAKNTVYLQMNSLKPEDTAMYYCKTDPLHCRAHGGSWYSVRANYWGQGTQVTVSSGGGSQVQLQESGGGSVEAGGSLRLSCAASGYTHSSYCMGWFRQAPGKEREGVAAIDVDGSTTYADSVKGRFTISKDNAKNTLYLQMNSLKPEDTGMYYCAAEFADCSSNYFLPPGAVRYWGQGTQVTVSS

SEQ ID NO: 790 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSS

SEQ ID NO: 791 - protein, 257 aa, synthetic construct
EVQLLESGGGLVQAGGSLRLSCAASGYSNCAYDMTWYRQAPGKEREFVSAIHSSGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSS

SEQ ID NO: 792 - protein, 257 aa, synthetic construct
EVQLLESGGGLVQAGGSLRLSCAASGYSNCAYDMTWYRQAPGKEREFVSAIHSDGSTYYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTTVTVSS

SEQ ID NO: 793 - protein, 257 aa, synthetic construct
EVQLLESGGGLVQAGGSLRLSCAASGYSNCAYDMTWYRQAPGKEREFVSAIHSDGSTRYADSVKGRFFISRDNSKNTVYLQMNSLRPEDTAVYYCKTDPLHCRAHGGSWYSVRANYWGQGTLVTVSSGGGSEVQLVESGGGLVQPGGSLRLSCAASGYTHSSYCMGWVRQAPGKGLEGVSAIDVDGSTTYADSVKGRFTISRDNSKNTLYLQMNSLRAEDTAVYYCAAEFADCSSNYFLPPGAVRYWGQGTLVTVSS

SEQ ID NO: 794 - protein, 229 aa, synthetic construct
GSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK